(19) United States
(12) Patent Application Publication
(10) Pub. No.: US 2024/0336594 A1
(43) Pub. Date: Oct. 10, 2024

(54) SIX-MEMBERED ARYL OR HETEROARYL AMIDES, AND COMPOSITION AND USE THEREOF

(71) Applicant: BETTA PHARMACEUTICALS CO., LTD, Hangzhou, Zhejiang (CN)

(72) Inventors: Bang FU, Beijing (CN); Yinlong LI, Beijing (CN); Wei REN, Beijing (CN); Qichao SHEN, Beijing (CN); Guolong DU, Beijing (CN); Xiaojun ZHOU, Beijing (CN); Yun SUN, Beijing (CN); Yuzhen ZHOU, Hangzhou, Zhejiang (CN); Xiao SUN, Beijing (CN); Weidong CAI, Hangzhou, Zhejiang (CN); Xiangyong LIU, Beijing (CN); Lieming DING, Hangzhou, Zhejiang (CN); Jiabing WANG, Beijing (CN)

(21) Appl. No.: 18/576,708

(22) PCT Filed: Jun. 24, 2022
(86) PCT No.: PCT/CN2022/101146

§ 371 Date: Jan. 4, 2024

(30) Foreign Application Priority Data
Jul. 5, 2021 (WO) PCT/CN2021/104458
Sep. 9, 2021 (WO) PCT/CN2021/117357
Apr. 12, 2022 (CN) 202210383102.0
Jun. 22, 2022 (CN) 202210715786.X

(51) Int. Cl. C07D 401/14; A61K 31/167; A61K 31/18; A61K 31/423; A61K 31/437; A61K 31/4375; A61K 31/44; A61K 31/443; A61K 31/4439; A61K 31/444; A61K 31/4545; A61K 31/4709; A61K 31/497; A61K 31/501; A61K 31/5025; A61K 31/506; A61K 31/519; A61K 31/538; C07C 237/40; C07C 311/32; C07C 317/32; C07C 323/33; C07D 205/12; C07D 213/56; C07D 263/56; C07D 265/36; C07D 401/04; C07D 401/06; C07D 405/12; C07D 405/14; C07D 409/12; C07D 413/04; C07D 413/12; C07D 417/04; C07D 471/04; C07D 487/04; C07D 487/08; C07D 491/048; C07D 491/107

(52) U.S. Cl. CPC C07D 401/14; A61K 31/167; A61K 31/18; A61K 31/423; A61K 31/437; A61K 31/4375; A61K 31/44; A61K 31/443; A61K 31/4439; A61K 31/444; A61K 31/4545; A61K 31/4709; A61K 31/497; A61K 31/501; A61K 31/5025; A61K 31/506; A61K 31/519; A61K 31/538; C07C 237/40; C07C 311/32; C07C 317/32; C07C 323/33; C07D 205/12; C07D 213/56; C07D 263/56; C07D 265/36; C07D 401/04; C07D 401/06; C07D 405/12; C07D 405/14; C07D 409/12; C07D 413/04; C07D 413/12; C07D 417/04; C07D 471/04; C07D 487/04; C07D 487/08; C07D 491/048; C07D 491/107

(57) ABSTRACT

A compound of formula I and a use thereof as an MALT1 inhibitor, and a pharmaceutical composition containing the compound. The compound can be used to treat diseases or disorders such as cancer.

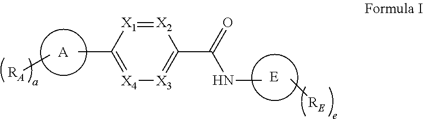

### FIELD OF THE INVENTION

The present invention belongs to the field of medicine, and specifically relates to a six-membered aryl or heteroaryl amide, and composition and use thereof.

### BACKGROUND OF THE INVENTION

MALT1 (mucosa-associated lymphoid tissue lymphoma translocation protein 1), as a key mediator of the classic NF-κB signaling pathway, is currently the only paracaspase known in humans that transduces signals from B cell receptors (BCR) and T cell receptors (TCR). The engagement of TCR/BCR results in the assembly of CARD11-BCL10-MALT1 (CBM) complex, which is composed of three proteins: CARD11 (caspase recruitment domain family member 11), BCL10 (B-cell lymphoma factor 10), and MALT1, to trigger the downstream activation. MALT1 affects NF-κB signaling through two mechanisms: first, MALT1 functions as a scaffolding protein, recruiting NF-κB signaling proteins such as TRAF6, TAB-TAK1 or NEMO-IKKα/μ; second, MALT1 use its protease function to cleave and inactivate negative regulators of NF-κB signaling, such as RelB, A20 or CYLD. The caspade signaling ultimately leads to the nuclear translocation of the NF-κB transcription factor complex and the activation of genes involved in proliferation, apoptosis inhibition, and inflammation (Jaworski et al., Cell Mol Life Sci, 2016, Vol. 73, 459-473).

Sustained activation of the NF-κB signaling pathway is a hallmark of ABC-DLBCL (activated B-cell-like subtype of diffuse large B-cell lymphoma). The ABC subtype is a more aggressive form of DLBCL. DLBCL is the most common form of non-Hodgkin lymphoma (NHL), accounting for approximately 25% of lymphoma cases, while ABC-DLBCL accounts for approximately 40% of DLBCL cases. In ABC-DLBCL patients, NF-κB pathway activation is driven by mutations in CD79A/B, CARD11, MYD88 or A20 (Staudt, ColdSpring Harb Perspect Biol, 2010, Vol. 2; Lim et al., Immunol Rev, 2012 Vol. 246, 359-378). Small molecule tool compound inhibitor of MALT1 protease has proven effective in preclinical models of ABC-DLBCL (Fontan et al., Cancer Cell, 2012, Vol. 22, 812-824; Nagel et al., Cancer ell, 2012, Vol. 22, 825-837).

In addition to lymphoma, MALT1 has also been shown to play a crucial role in both innate and adaptive immunity (Jaworski M et al., Cell Mol Life Sci., 2016). MALT1 protease inhibitors showed the anti-inflammatory activity by slowing down the disease onset and progression of experimental allergic encephalomyelitis (mouse model of multiple sclerosis) in mice (Mc Guire et al., J. Neuroinflammation, 2014, Vol. 11, 124). Furthermore, MALT1 knockout mice have fewer Tregs with Teff cell function severely impaired, and the immunity is generally compromised. In contrast, only knockout of the protease domain of MALT1 significantly diminishes Tregs but preservers the functions of Teff cell. Therefore, MALT1 protease inhibitors can benefit patients with solid tumors by regulating Treg function and promoting the body's immune response (Arianna Bertossi et al., EMBO J, 2014, Vol. 33, 2740-2742).

Therefore, the MALT1 inhibitors of the present invention may provide beneficial therapeutic effects to patients suffering from cancer and/or immune diseases.

### SUMMARY OF INVENTION

The present invention relates to a six-membered aryl or heteroaryl amide and its composition and use, which can be used as an inhibitor of MALT1 for the treatment of cancer and/or immune-related diseases.

A first object of the present invention is to provide a compound of Formula I, or a stereoisomer, tautomer, deuterated compound, pharmaceutically acceptable salt, prodrug, chelate, non-covalent complex or solvate thereof,

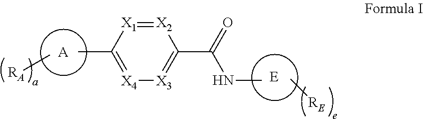

wherein,
X₁ is selected from CR₁ or N;
X₂ is selected from CR₂ or N;
X₃ is selected from CR₃ or N;
X₄ is selected from CR₄ or N;
R₁, R₂, R₃, R₄ are the same or different, and are each independently selected from the group consisting of H, CN, NO₂, halogen, —C₁₋₆ alkyl, —C₂₋₆ alkenyl, —C₂₋₆ alkynyl, —OR₅, —NR₅R₆, SR₅, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, —C(O)R₅, —N(R₅)C(O)R₆, —C(O)NR₅R₆, —N(R₅)C(O)OR₆, —OC(O)NR₅R₆, —N(R₅)C(O)NR₆R₇, —S(O)R₅, —S(O)₂R₅, —S(O)NR₅R₆, —S(O)₂NR₅R₆, —N(R₅)S(O)R₆, —N(R₅)S(O)₂R₆, —C(O)OR₅, —OC(O)R₅ and —OC(O)OR₅; wherein the —C₁₋₆ alkyl, —C₂₋₆ alkenyl, —C₂₋₆ alkynyl, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, R₅, R₆ and R₇ are optionally substituted with one or more substituents selected from hydrogen, halogen, CN, —OR₈, —NR₈R₉, —SR₈, —C(O)R₈, —N(R₈)C(O)R₉, —C(O)NR₈R₉, —N(R₈)C(O)OR₉, —OC(O)NR₈R₉, —N(R₈)C(O)NR₉R₁₀, —S(O)R₈, —S(O)₂R₈, —S(O)NR₈R₉, —S(O)₂NR₈R₉, —N(R₈)S(O)R₉, —N(R₈)S(O)₂R₉, —C(O)OR₈, —OC(O)R₈, —OC(O)OR₈, oxo, —C₁₋₆ alkyl, —C₂₋₆ alkenyl, —C₂₋₆ alkynyl, —C₀₋₆ alkyl-C₃₋₈ carbocyclyl or —C₀₋₆ alkyl-3-8 membered heterocyclyl;
ring A is selected from —C₅₋₆ carbocyclyl, 5-6 membered heterocyclyl, —C₆ aryl or 5-6 membered heteroaryl;
each R_{A} is the same or different, and is independently selected from the group consisting of hydrogen, halogen, oxo, CN, NO₂, —C₁₋₆ alkyl, —C₂₋₆ alkenyl, —C₂₋₆ alkynyl, —OR₅, —NR₅R₆, —SR₅, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, —C(O)R₅, —N(R₅)C(O)R₆, —C(O)NR₅R₆, —N(R₅)C(O)OR₆, —OC(O)NR₅R₆, —N(R₅)C(O)NR₆R₇, —S(O)R₅, —S(O)₂R₅, —S(O)NR₅R₆, —S(O)₂NR₅R₆, —N(R₅)S(O)R₆, —N(R₅)S(O)₂R₆, —C(O)OR₅, —OC(O)R₅ and —OC(O)OR₅; wherein the —C₁₋₆ alkyl, —C₂₋₆ alkenyl, —C₂₋₆ alkynyl, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, R₅, R₆ and R₇ are optionally substituted with one or more substituents selected from hydrogen, halogen, CN, oxo, —C₁₋₆ alkyl, —C₂₋₆ alkenyl, —C₂₋₆ alkynyl, —C₀₋₆ alkyl-C₃₋₈ carbocyclyl, —C₀₋₆ alkyl-3-8 membered heterocyclyl, —OR₈, —NR₈R₉, —SR₈, —S(O)R₈, —S(O)₂R₈, —C(O)R₈, —C(O)OR₈, —OC(O)R₈, —N(R₈)C(O)R₉ or —C(O)NR₈R₉;
ring E is selected from 5-10 membered heterocyclyl, C₆₋₁₀ aryl or 5-10 membered heteroaryl;
each R_{E} is the same or different, and is independently selected from the group consisting of hydrogen, halogen, oxo, CN, NO₂, —C₁₋₆ alkyl, —C₂₋₆ alkenyl, —C₂₋₆ alkynyl, —C₀₋₆ alkyl-5-10 membered heteroaryl, —C₀₋₆ alkyl-C₃₋₈ carbocyclyl, —C₀₋₆ alkyl-3-8 membered heterocyclyl, —OR₅, —NR₅R₆, —SR₅, —C(O)R₅, —N(R₅)C(O)R₆, —C(O)NR₅R₆, —N(R₅)C(O)OR₆, —OC(O)NR₅R₆, —N(R₅)C(O)NR₆R₇, —S(O)R₅, —S(O)₂R₅, —S(O)NR₅R₆, —S(O)₂NR₅R₆, —N(R₅)S(O)R₆, —N(R₅)S(O)₂R₆, —C(O)OR₅, —OC(O)R₆ and —OC(O)OR₇; wherein the —C₁₋₆ alkyl, —C₂₋₆ alkenyl, —C₂₋₆ alkynyl, —C₀₋₆ alkyl-5-10 membered heteroaryl, —C₀₋₆ alkyl-C₃₋₈ carbocyclyl, —C₀₋₆ alkyl-3-8 membered heterocyclyl, R₅, R₆ and R₇ are optionally substituted with one or more substituents selected from hydrogen, halogen, CN, oxo, —C₁₋₆ alkyl, —C₂₋₆ alkenyl, —C₂₋₆ alkynyl, —OR₈, —NR₈R₉, substituted or unsubstituted —C₀₋₆ alkyl-C₃₋₆ carbocyclyl, substituted or unsubstituted —C₀₋₆ alkyl-3-6 membered heterocyclyl, —C(O)OR₈ or —C(O)NR₈R₉;
R₅, R₆, R₇, R₈, R₉, R₁₀ are each independently selected from the group consisting of hydrogen, halogen, CN, OH, NH₂, oxo, —C₁₋₆ alkyl, —C₁₋₆ alkoxy, —C₂₋₆ alkenyl, —C₂₋₆ alkynyl, —C₀₋₆ alkyl-C₃₋₁₄ carbocyclyl, —C₀₋₆ alkyl-3-14 membered heterocyclyl; wherein the —C₁₋₆ alkyl, —C₁₋₆ alkoxy, —C₂₋₆ alkenyl, —C₂₋₆ alkynyl, —C₀₋₆ alkyl-C₃₋₁₄ carbocyclyl, —C₀₋₆ alkyl-3-14 membered heterocyclyl are optionally substituted with one or more substituents selected from hydrogen, halogen, CN, OH, NH₂, —COOH, oxo, —C₁₋₆ alkyl, —C₁₋₆ haloalkyl, —C₂₋₆ alkenyl, —C₂₋₆ alkynyl, —C₁₋₆ alkoxy, —C₁₋₆ haloalkoxy, —NH(C₁₋₆ alkyl), —N(C₁₋₆ alkyl)₂, —COO(C₁₋₆ alkyl), —CONH(C₁₋₆ alkyl), —CON(C₁₋₆ alkyl)₂, substituted or unsubstituted —C₀₋₆ alkyl-C₃₋₆ carbocyclyl, substituted or unsubstituted —C₀₋₆ alkyl-3-6 membered heterocyclyl;
a is selected from 0, 1, 2, 3, 4, 5 or 6;
e is selected from 0, 1, 2, 3, 4, 5 or 6;
when X₁ is selected from CH, X₂ is selected from CH, X₃ is selected from CH, and X₄ is selected from CH,

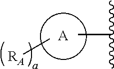

is not phenyl.

In some embodiments of Formula I,

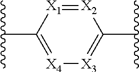

is selected from

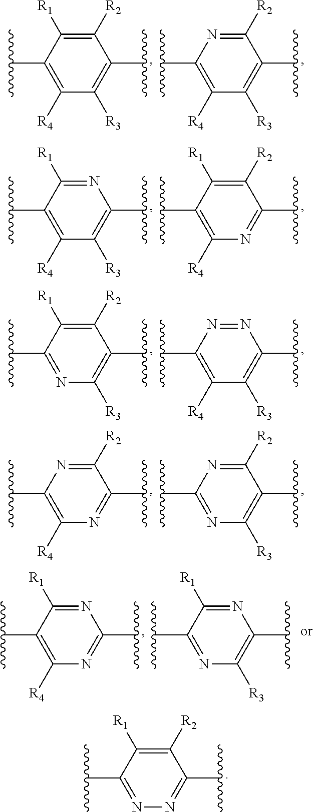

In some embodiments of Formula I,

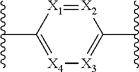

is selected from

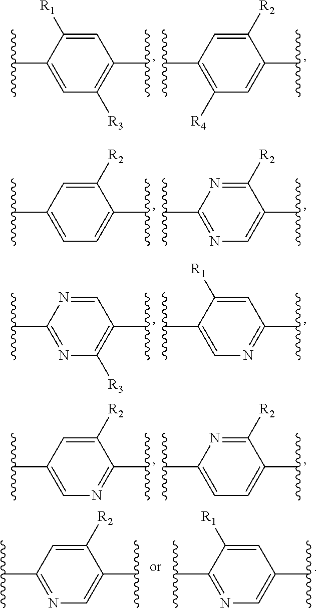

In some embodiments of Formula I, R₁, R₃ are the same or different, and are each independently selected from the group consisting of halogen, —C₁₋₆ alkyl, —OR₅, —NR₅R₆, —C₀₋₆ alkyl —C₃₋₈ carbocyclyl and —C₀₋₆ alkyl-3-8 membered heterocyclyl; wherein the —C₁₋₆ alkyl, —C₀₋₆ alkyl —C₃₋₈ carbocyclyl, —C₀₋₆ alkyl-3-8 membered heterocyclyl, R₅ and R₆ are optionally substituted with one or more substituents selected from hydrogen, halogen, CN, OH, NH₂, —C₁₋₆ alkyl, —C₂₋₆ alkenyl, —C₂₋₆ alkynyl, —C₁₋₆ alkoxy, —C₀₋₆ alkyl —C₃₋₈ carbocyclyl or —C₀₋₆ alkyl-3-8 membered heterocyclyl.

In some embodiments of Formula I, R₂, R₄ are the same or different, and are each independently selected from the group consisting of halogen, —C₁₋₆ alkyl, —OR₅, —NR₅R₆, —C₀₋₆ alkyl —C₃₋₈ carbocyclyl and —C₀₋₆ alkyl-3-8 membered heterocyclyl; wherein the —C₁₋₆ alkyl, —C₀₋₆ alkyl —C₃₋₈ carbocyclyl, —C₀₋₆ alkyl-3-8 membered heterocyclyl, R₅ and R₆ are optionally substituted with one or more substituents selected from hydrogen, halogen, CN, OH, NH₂, —C₁₋₆ alkyl, —C₂₋₆ alkenyl, —C₂₋₆ alkynyl, —C₁₋₆ alkoxy, —C₀₋₆ alkyl —C₃₋₈ carbocyclyl or —C₀₋₆ alkyl-3-8 membered heterocyclyl.

In some embodiments of Formula I, R₁, R₂, R₃, R₄ are the same or different, and are each independently selected from the group consisting of H, —F, —Cl, —Br, —I, —OH, —NH₂, —CN, —CH₃, —CF₃, —CH₂CH₃, —CH(CH₃)₂, —CH₂CH(CH₃)₂, —C(CH₃)₃, —NHCH(CH₃)₂, —NHC(O)CH₃, —NHC(O)OCH₂CH₃,

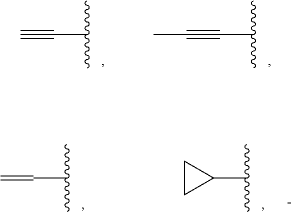

OCH₃, —OCH(CH₃)₂,

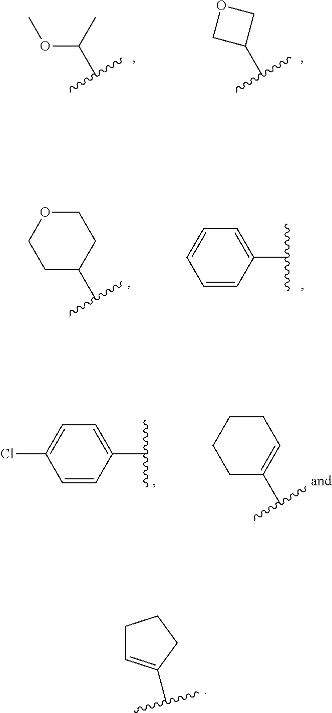

In some embodiments of Formula I,

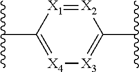

is selected from

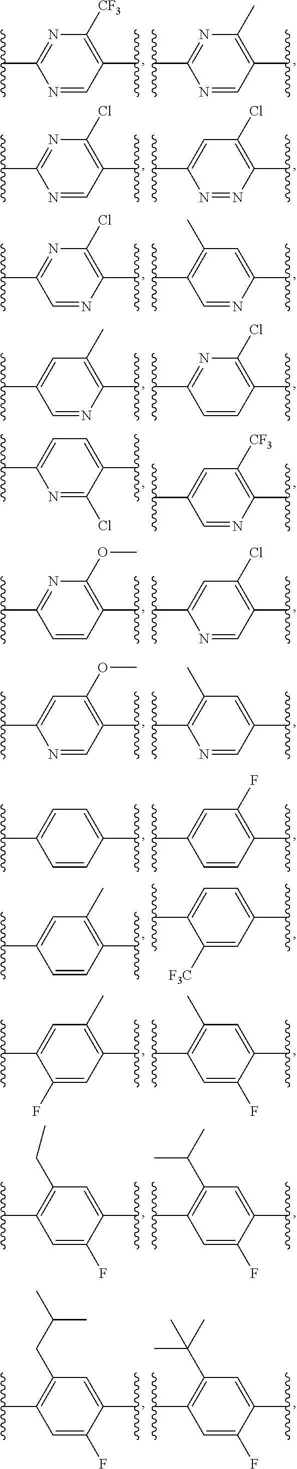

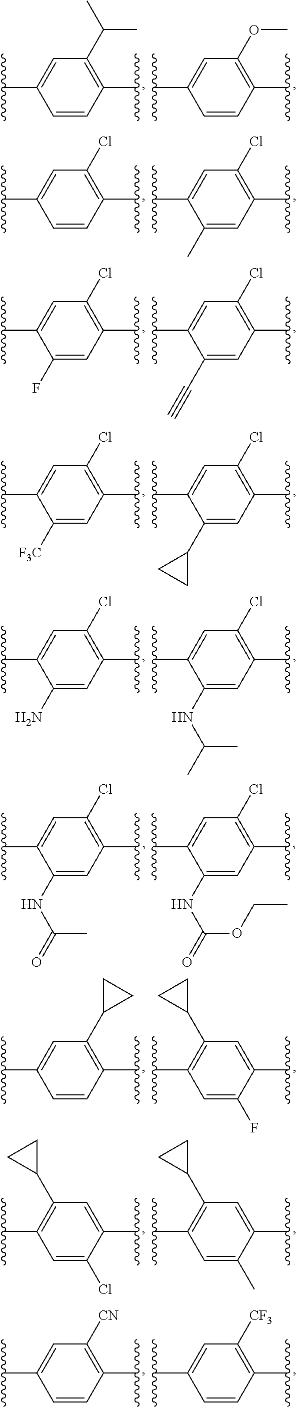

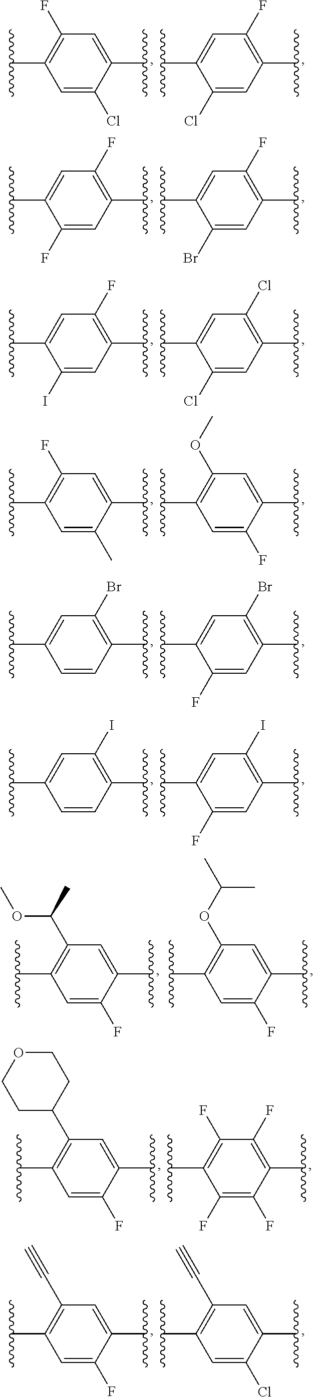

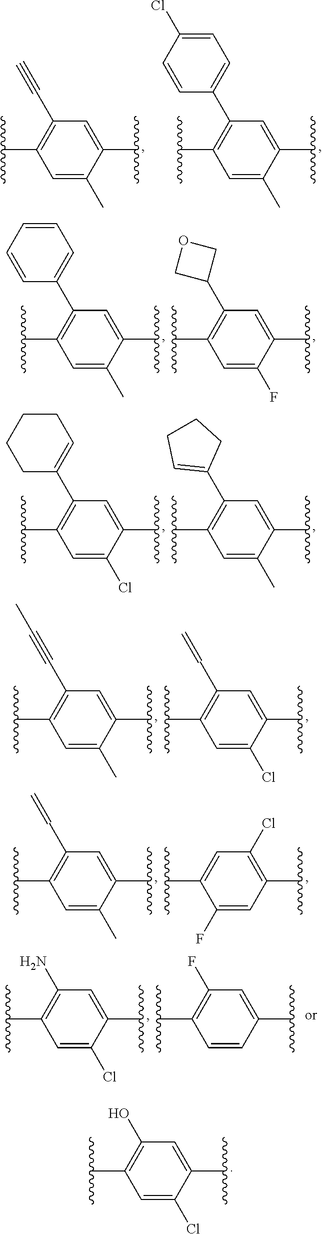

In some embodiments of Formula I, ring A is selected from —C₆ carbocyclyl, membered heterocyclyl, —C₆ aryl or 6 membered heteroaryl.

In some embodiments of Formula I, ring A is selected from Phenyl, pyridyl, furyl, thienyl, pyrrolyl, pyrazolyl, pyrimidinyl, pyridazinyl, diazinyl, cyclopentenyl, pyridin-2(1H)-one or pyrimidine-2(1H)-keto group, etc.

In some embodiments of Formula I, ring A is

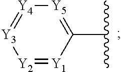

wherein Y₁ is selected from CR_{A} or N; Y₂ is selected from CR_{A} or N; Y₃ is selected from CR_{A} or N; Y₄ is selected from CR_{A} or N; R_{A} is defined as described for the compound represented by formula I above.

In some embodiments of Formula I, each R_{A} is the same or different, and is independently selected from the group consisting of hydrogen, oxo, —F, —Cl, —Br, —I, —CN, —OH, —NH₂, —NO₂, —CH₃, —CF₃, —OCF₃, —NHCH₃, —N(CH₃)₂, —OCH₃, —SCH₃, —CHF₂, —OCHF₂, —C(O)NH₂, —C(O)CH₃, —OC(O)NH₂, —NHC(O)CH₃, —CH₂CH₃, —CH(CH₃)₂,

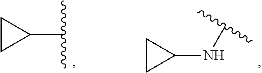

—SCH₃, —SOCH₃, —CH₂SO₂CH₃, —SO₂CH₃, —NHC(O)NH₂, —CH₂OH, —CH₂NH₂, —NHC(O)OCH₂CH₃, —NHOH, —CH₂OCH₃,

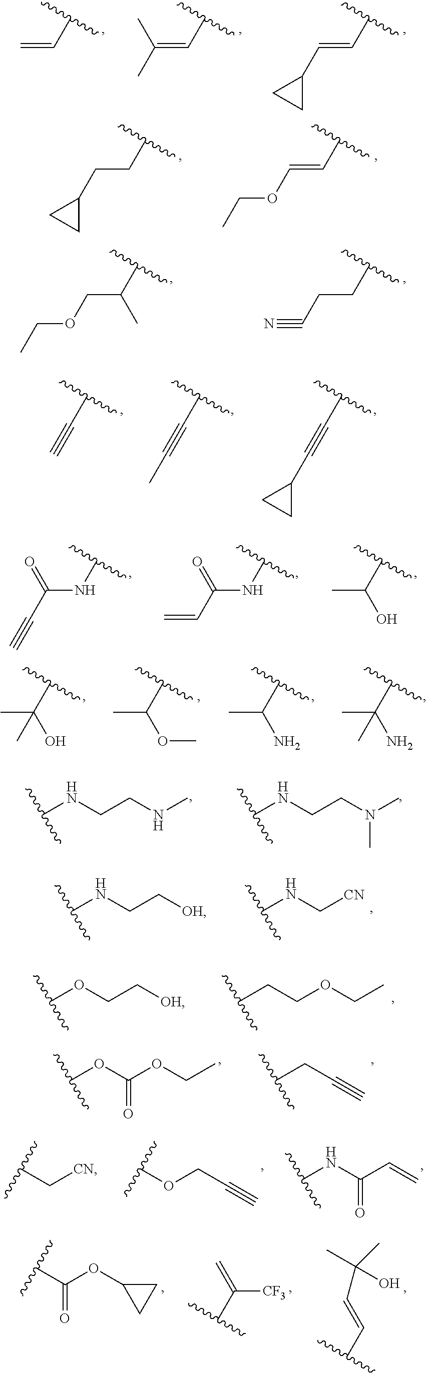

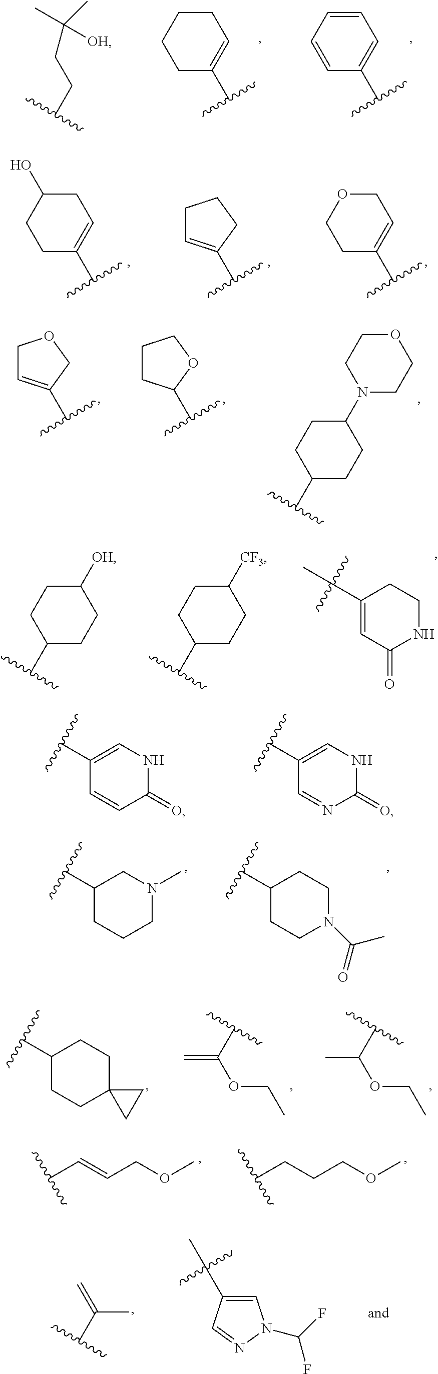

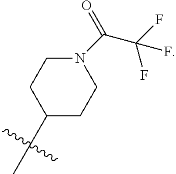 [SMILES: *C1CCN(C(=O)C(F)(F)F)CC1]

In some embodiments of Formula I,

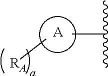

is selected from

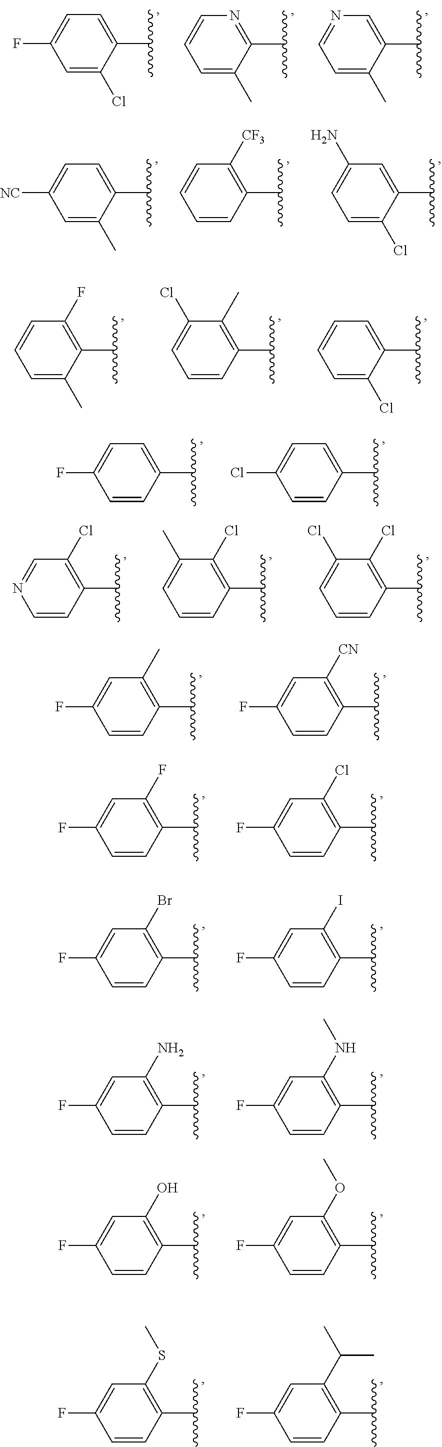

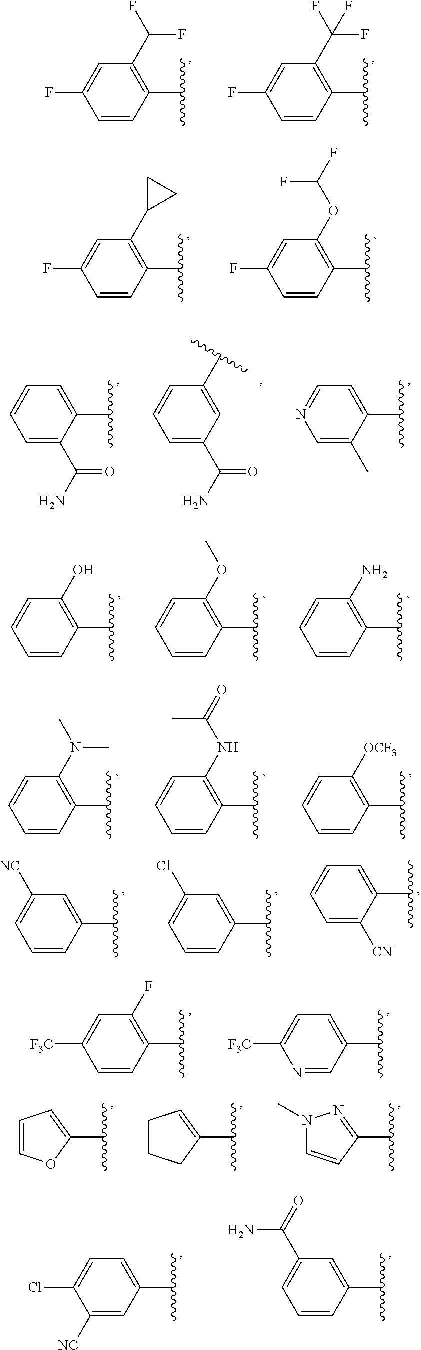

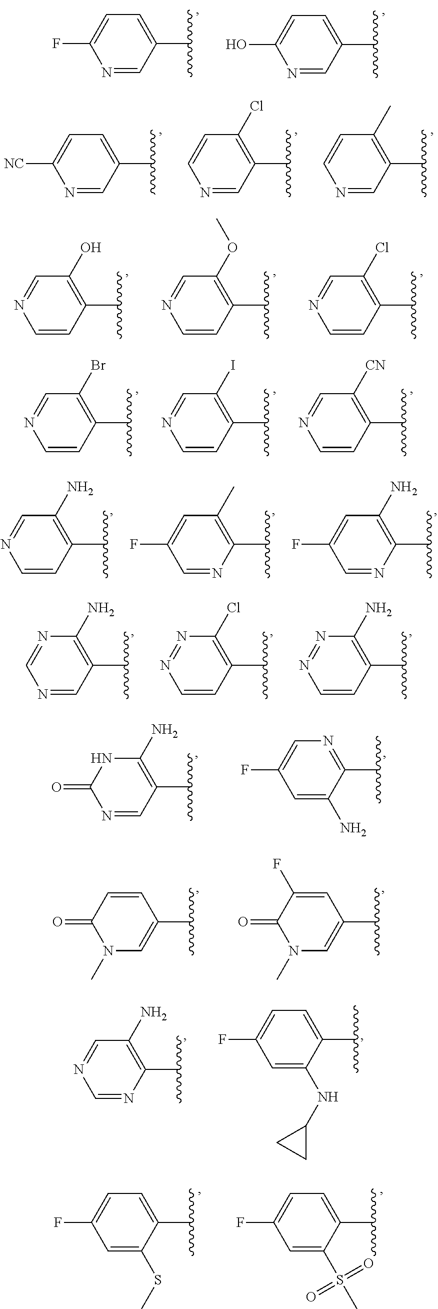

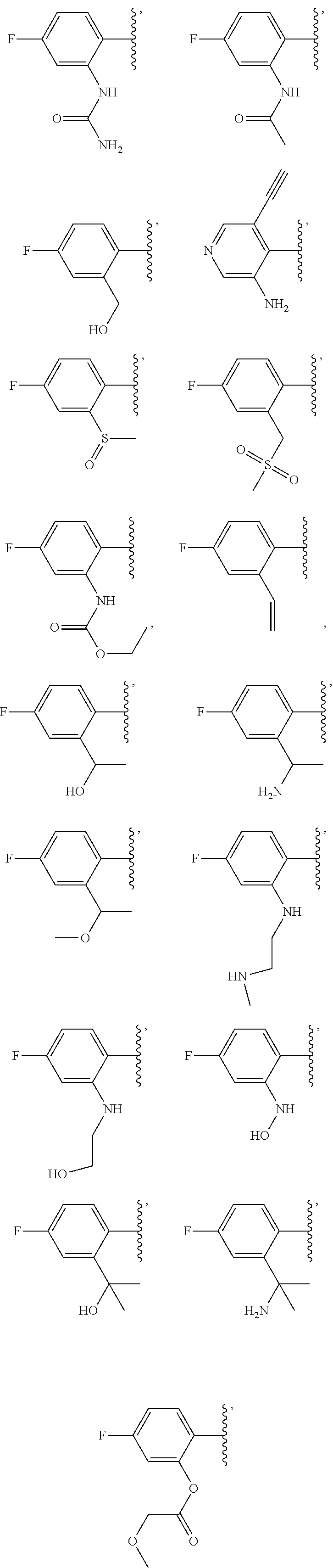

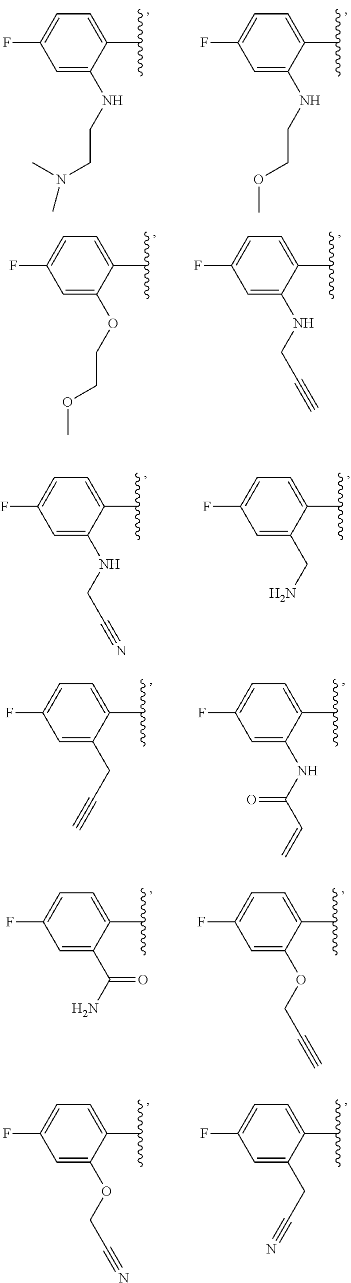

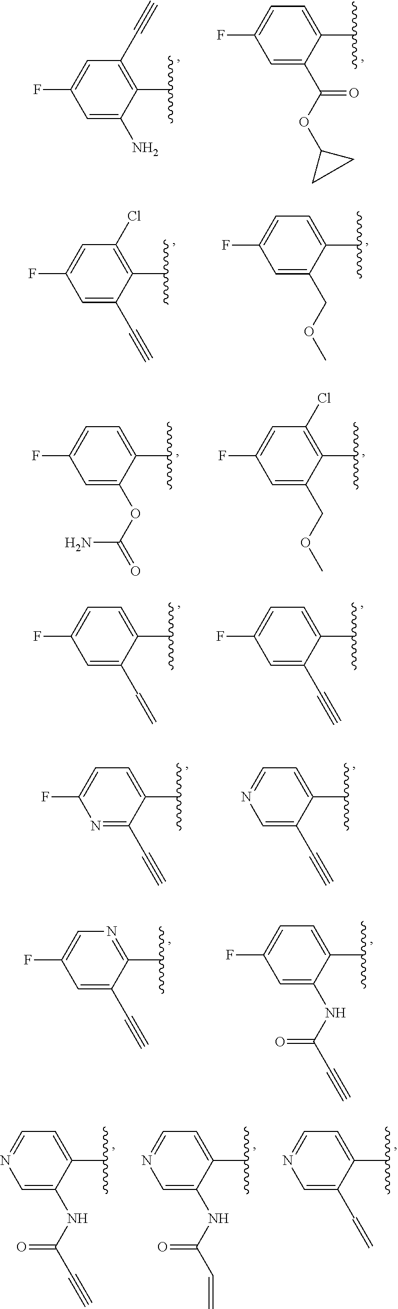

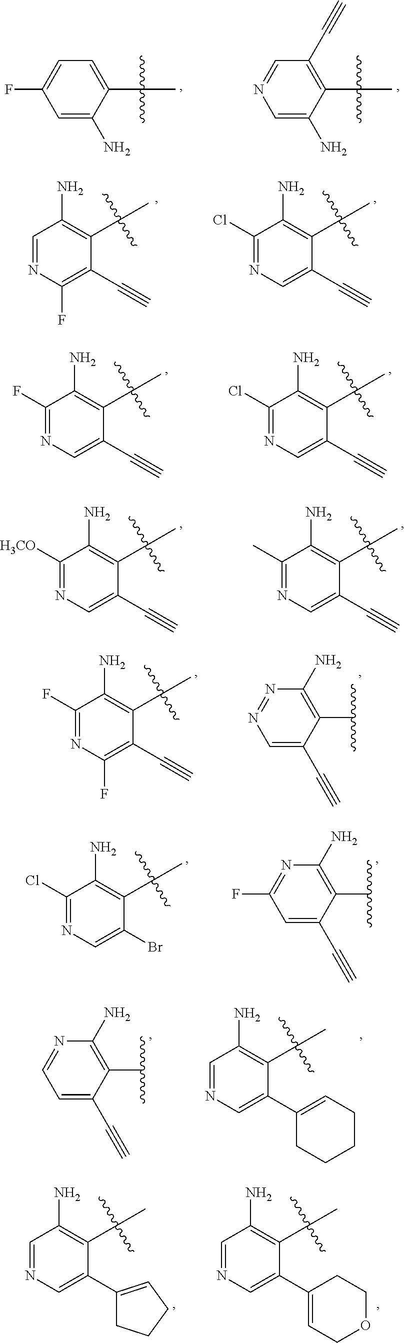

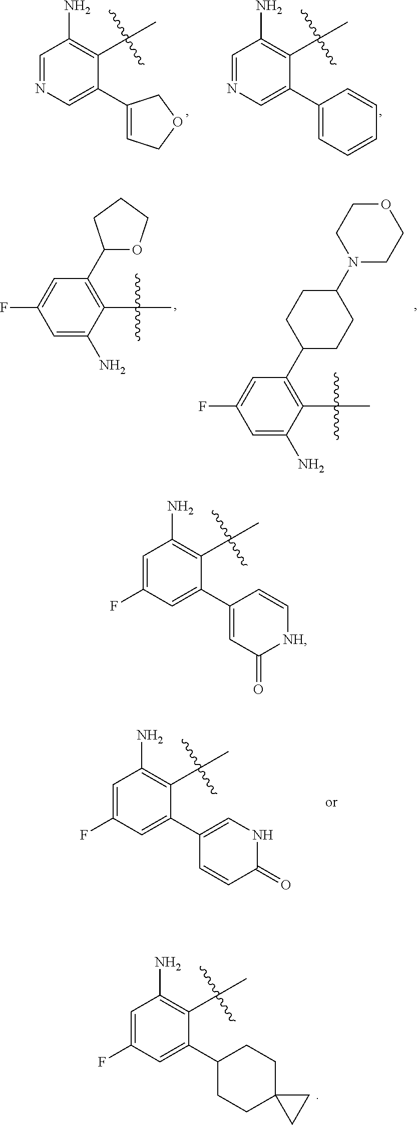

In some embodiments of Formula I, ring E is selected from 6-10 membered heterocyclyl or 6-10 membered heteroaryl.

In some embodiments of Formula I, ring E is selected frompyridyl, benzo[d]oxazolyl, 3,4-dihydro-2H-benzo[b][1,4]oxazinyl, 1,6-naphthyridinyl, 2,3-dihydro-1H-pyrido[2,3-b][1,4]oxazinyl, 1H-pyrazolo[4,3-b]pyridyl, 2H-pyrazolo[4,3-b]pyridyl, 1H-pyrazolo[3,4-b]pyridyl, pyrazolo[1,5-a]pyrimidinyl, imidazo[1,2-b]pyridazinyl, thiazolo[5,4-b] pyridyl or 1,5-naphthyridinyl.

In some embodiments of Formula I, each R_{E} is the same or different, and is independently selected from the group consisting of hydrogen, halogen, oxo, CN, —C₁₋₆ alkyl, —C₀₋₆ alkyl-5-10 membered heteroaryl, —C₀₋₆ alkyl —C₃₋₈ carbocyclyl, —C₀₋₆ alkyl-3-8 membered heterocyclyl, —OR₅, —NR₅R₆, —SR₅, —C(O)R₅, —S(O)R₅, —S(O)₂R₅ and —C(O)OR₅; wherein the —C₁₋₆ alkyl, —C₀₋₆ alkyl-5-10 membered heteroaryl, —C₀₋₆ alkyl —C₃₋₈ carbocyclyl and —C₀₋₆ alkyl-3-8 membered heterocyclyl are optionally substituted with one or more substituents selected from hydrogen, halogen, CN, oxo, —C₁₋₆ alkyl, —C₂₋₆ alkenyl, —C₂₋₆ alkynyl, —OR₈, —NR₈R₉, —C₀₋₆ alkyl —C₃₋₈ carbocyclyl and —C₀₋₆ alkyl-3-8 membered heterocyclyl, —C(O)OR₈ or —C(O)NR₈R₉.

In some embodiments of Formula I, each R_{E} is the same or different, is independently selected from the group consisting of hydrogen, oxo, —F, —Cl, —CN, —CH₃, —OCH₃, —CH₂OH, —CH₂CN, —CF₃, —C(O)NH₂, —C(O)NHCH₃, —C(O)NHCH₂CN, —C(O)NHCH₂CH₃, —C(O)NHCH₂CF₃, —C(O)NHCH₂CH₂OCF₃, —C(O)NHCH₂CH₂CH₃, —OCHF₂, —OCF₃, —SO₂CH₃, —SCH₃, —SCF₃, —NHC(O)NH₂, —NHC(O)CH₃, —NHC(O)OCH₃, —NHS(O)₂CH₃,

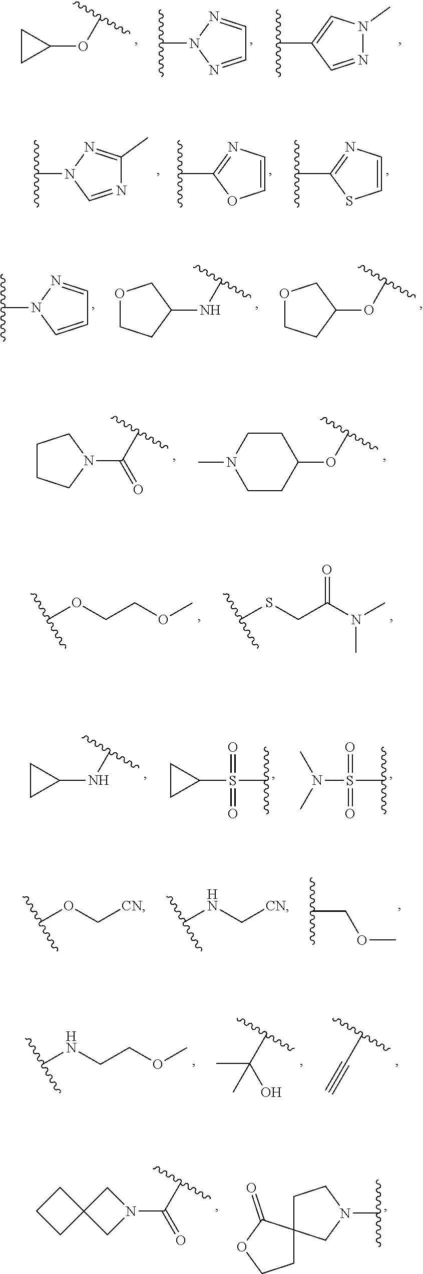

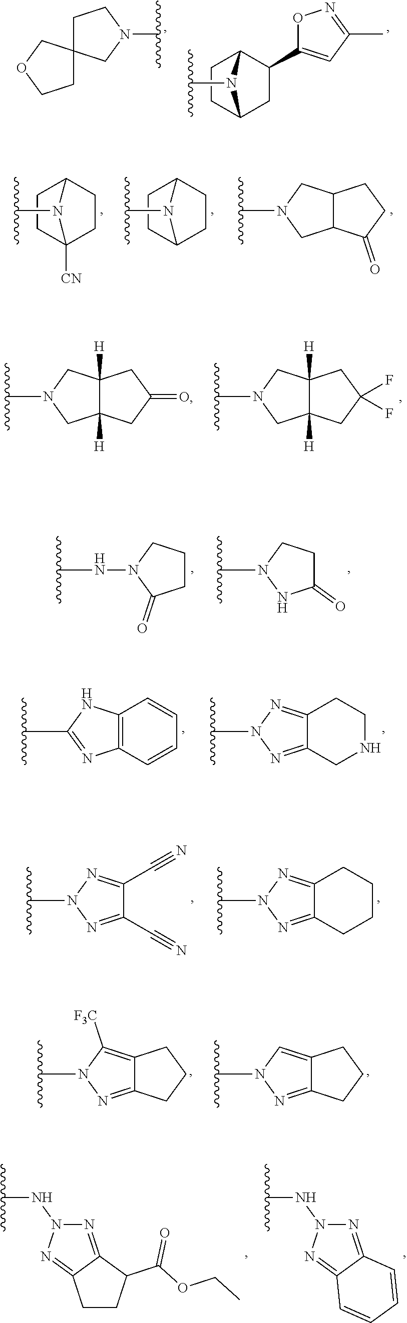

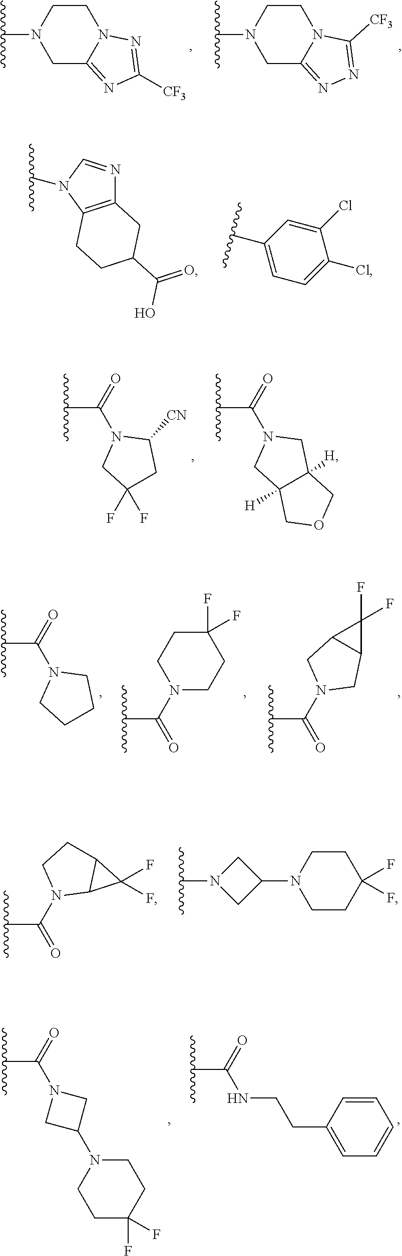

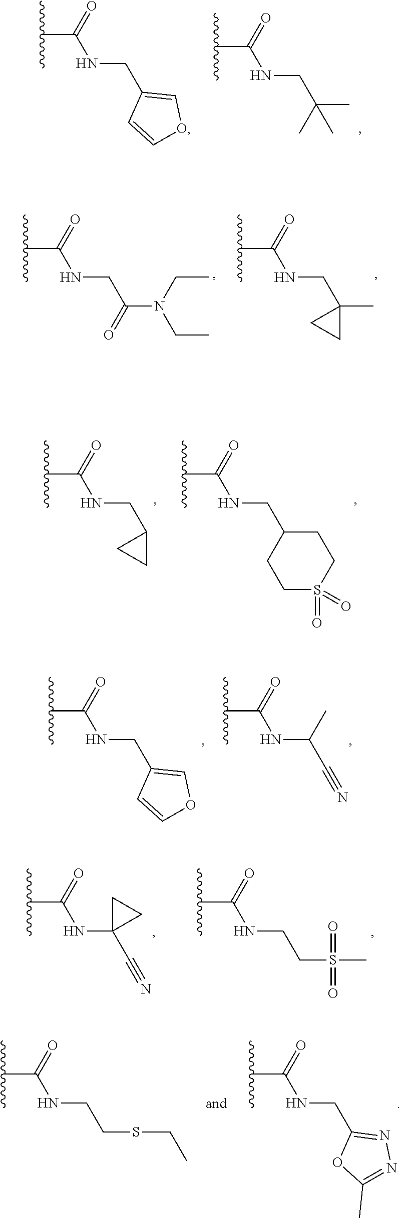

In some embodiments of Formula I,

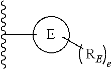

is selected from

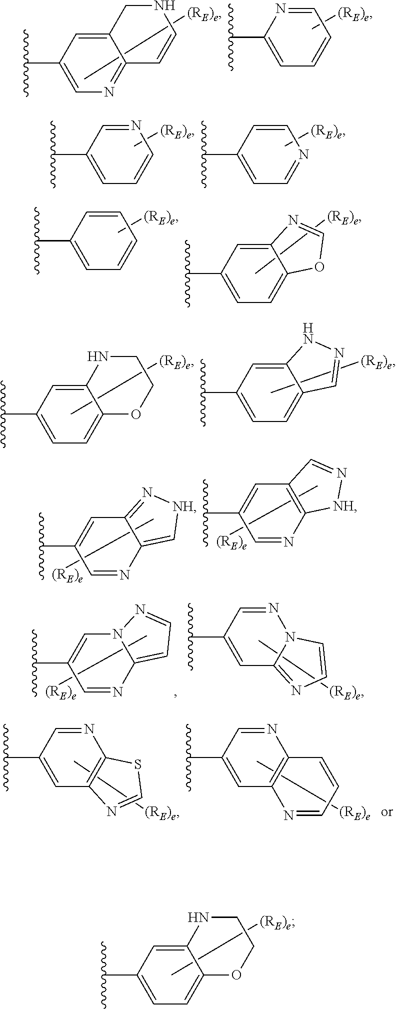

preferably,

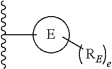

is selected from

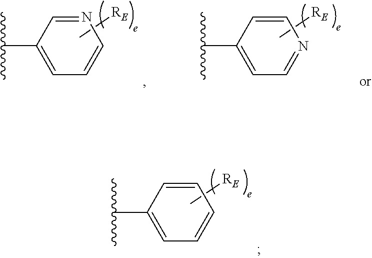

more preferably,

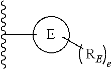

is selected from

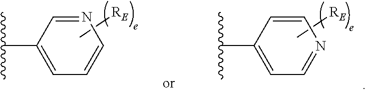

Wherein R_{E} and e are defined as described in any embodiment of the present invention.

In some embodiments of Formula I

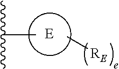

is selected from

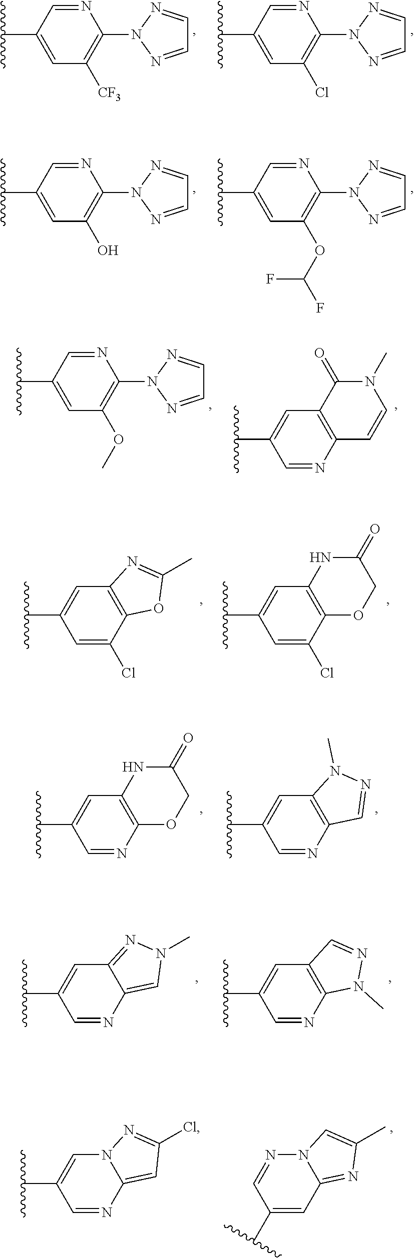

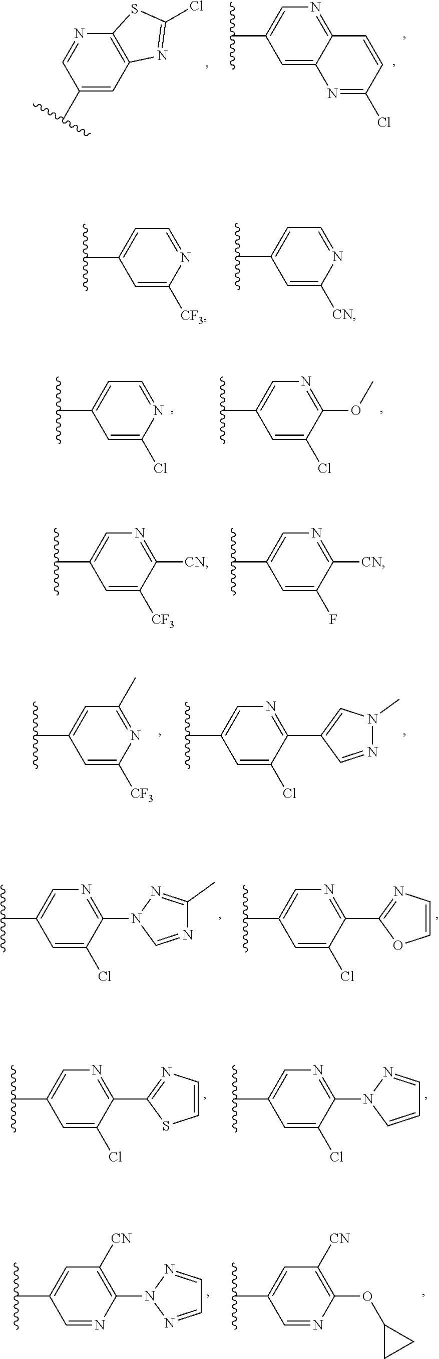

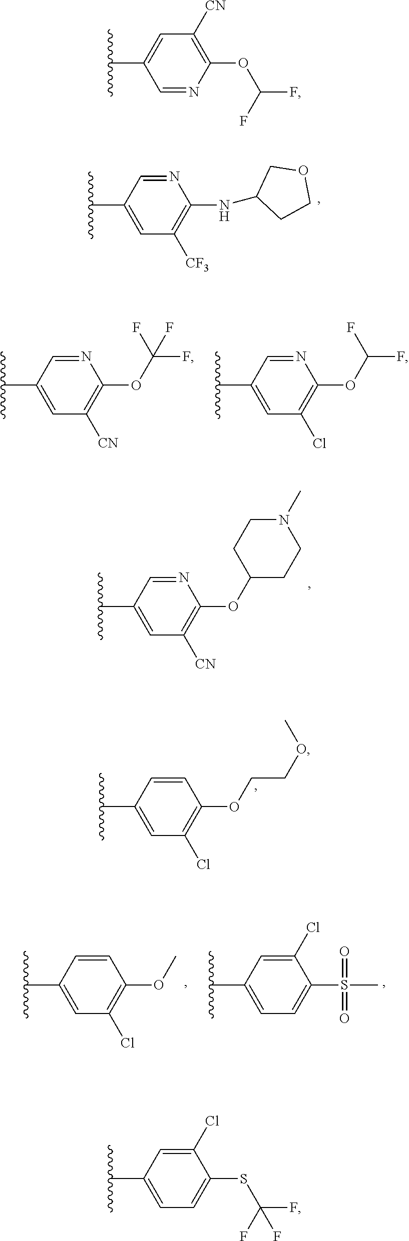

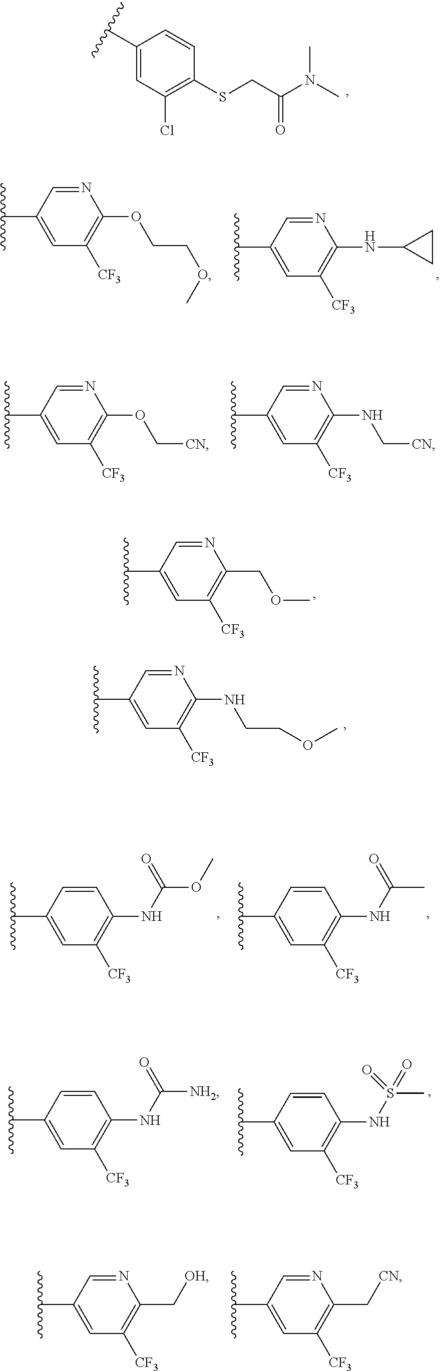

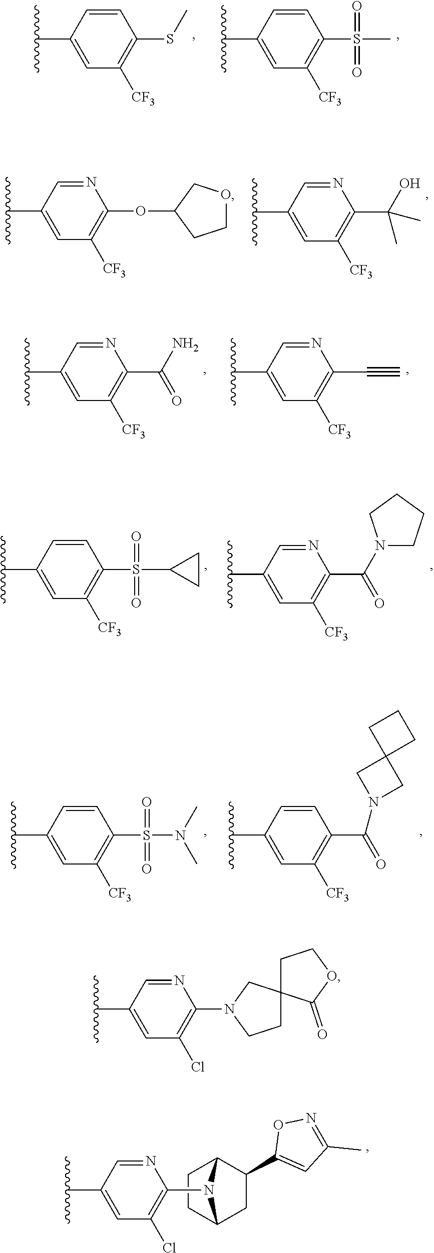

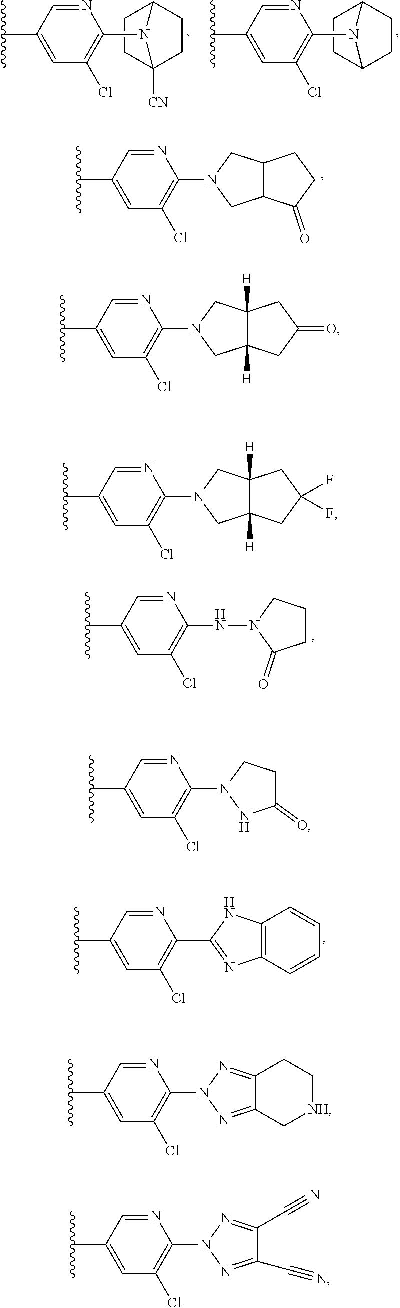

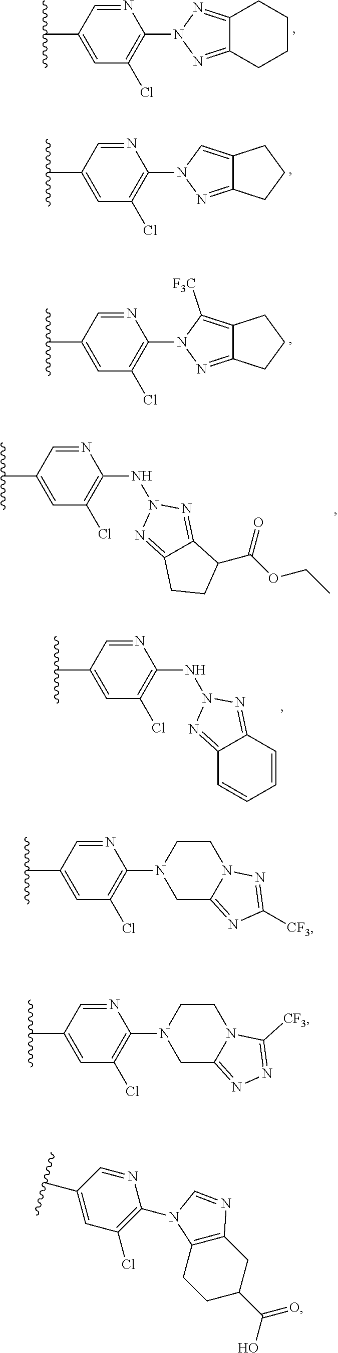

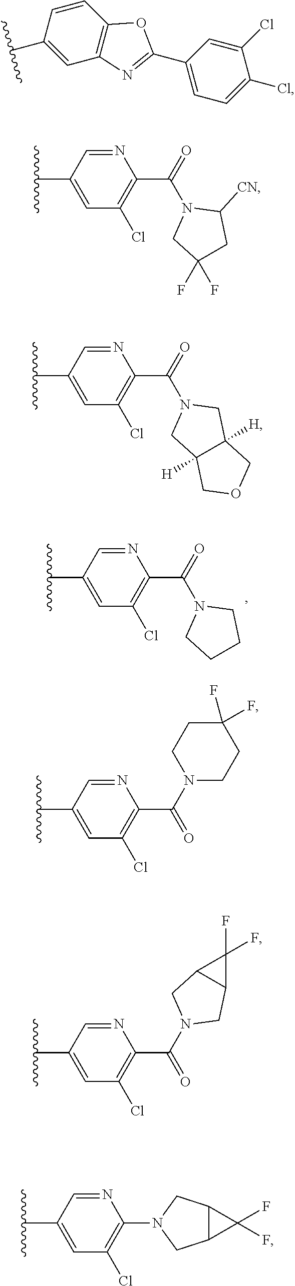

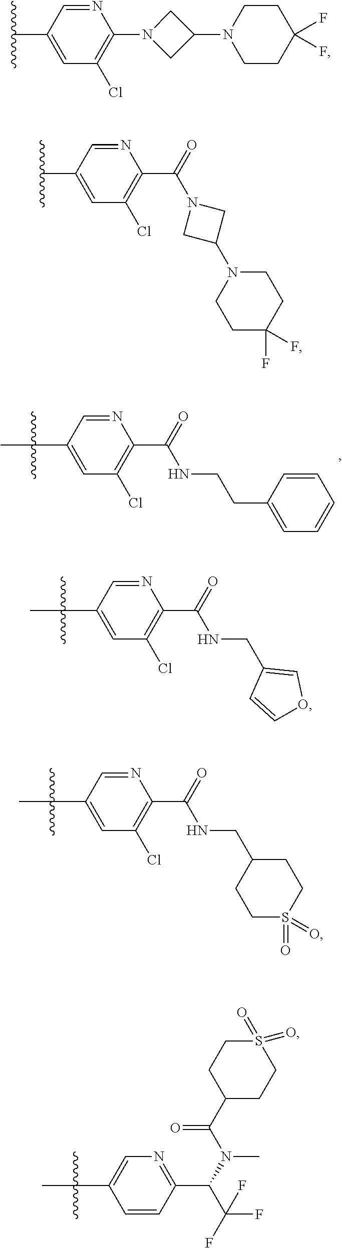 [SMILES: *c1cnc(N2CC(N3CCC(F)(F)CC3)C2)c(Cl)c1 ; *c1cnc(C(=O)N2CC(N3CCC(F)(F)CC3)C2)c(Cl)c1 ; *c1cnc(C(=O)NCCc2ccccc2)c(Cl)c1 ; *c1cnc(C(=O)NCc2ccoc2)c(Cl)c1 ; *c1cnc(C(=O)NCC2CCS(=O)(=O)CC2)c(Cl)c1 ; *c1ccc([C@H](N(C)C(=O)C2CCS(=O)(=O)CC2)C(F)(F)F)nc1]

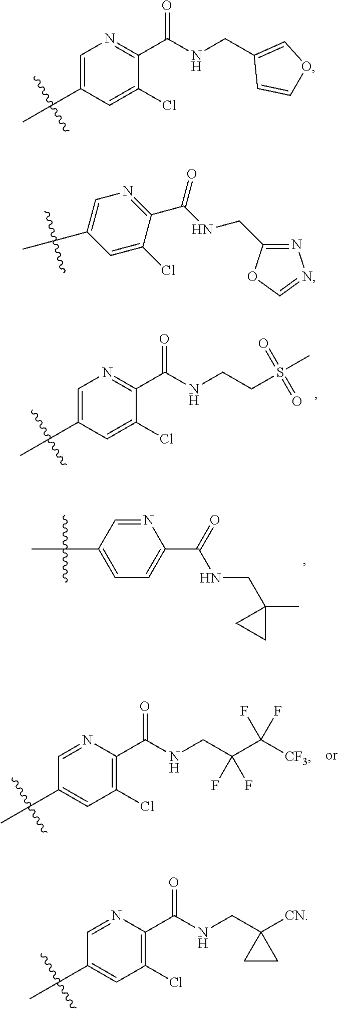

In some embodiments of Formula I, the aforementioned —C₃₋₈ carbocyclyl, 3-8 membered heterocyclyl can be respectively preferably —C₃₋₆ carbocyclyl, 3-6 membered heterocyclyl.

In some embodiments, the compound of Formula I is selected from Formula II:

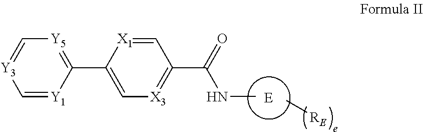

wherein, ring E, X₁, X₃, Y₁, Y₃, Y₅, R_{E}, e are defined as described in any embodiment of the invention.

In some embodiments, the compound of Formula I is selected from III:

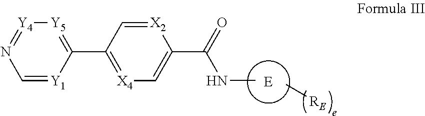

wherein, ring E, X₂, X₄, Y₁, Y₄, Y₅, R_{E}, e are defined as described in any embodiment of the invention.

In some embodiments of Formula I, the compound is selected from:
N-(6-(2H-1,2,3-triazol-2-yl)-5-(trifluoromethyl)pyridin-3-yl)-5-(2-chloro-4-fluorophenyl)-4-methylpicolinamide;
N-(6-(2H-1,2,3-triazol-2-yl)-5-(trifluoromethyl)pyridin-3-yl)-2-fluoro-4-(3-methylpyridin-2-yl)benzamide;
N-(6-(2H-1,2,3-triazol-2-yl)-5-(trifluoromethyl)pyridin-3-yl)-2′-chloro-4′-fluoro-3-isopropyl-[1,1′-biphenyl]-4-carboxamide;
N-(6-(2H-1,2,3-triazol-2-yl)-5-(trifluoromethyl)pyridin-3-yl)-3,5′-dimethyl-[2,3′-bipyridine]-6′-carboxamide;
N-(6-(2H-1,2,3-triazol-2-yl)-5-(trifluoromethyl)pyridin-3-yl)-2-methoxy-4-(3-methylpyridin-2-yl)benzamide;
N-(6-(2H-1,2,3-triazol-2-yl)-5-(trifluoromethyl)pyridin-3-yl)-2′-chloro-4′-fluoro-3-methoxy-[1,1′-biphenyl]-4-carboxamide;
N-(6-(2H-1,2,3-triazol-2-yl)-5-(trifluoromethyl)pyridin-3-yl)-6-(2-chloro-4-fluorophenyl)-2-methoxynicotinamide;
N-(6-(2H-1,2,3-triazol-2-yl)-5-(trifluoromethyl)pyridin-3-yl)-2-(2-chloro-4-fluorophenyl)-4-methylpyrimidine-5-carboxamide;
N-(6-(2H-1,2,3-triazol-2-yl)-5-(trifluoromethyl)pyridin-3-yl)-4-methyl-2-(4-methylpyridin-3-yl)pyrimidine-5-carboxamide;
N-(6-(2H-1,2,3-triazol-2-yl)-5-(trifluoromethyl)pyridin-3-yl)-2′-chloro-4′-fluoro-3-methyl-[1,1′-biphenyl]-4-carboxamide;
N-(6-(2H-1,2,3-triazol-2-yl)-5-(trifluoromethyl)pyridin-3-yl)-2′,3-dichloro-4′-fluoro-[1,1′-biphenyl]-4-carboxamide;
N-(6-(2H-1,2,3-triazol-2-yl)-5-(trifluoromethyl)pyridin-3-yl)-4-chloro-6-(2-chloro-4-fluorophenyl)nicotinamide;
N-(6-(2H-1,2,3-triazol-2-yl)-5-(trifluoromethyl)pyridin-3-yl)-2′-chloro-3-cyclopropyl-4′-fluoro-[1,1′-biphenyl]-4-carboxamide;
N-(6-(2H-1,2,3-triazol-2-yl)-5-(trifluoromethyl)pyridin-3-yl)-2-(4-cyano-2-methylphenyl)-4-methylpyrimidine-5-carboxamide;
N-(6-(2H-1,2,3-triazol-2-yl)-5-(trifluoromethyl)pyridin-3-yl)-5-(2-chloro-4-fluorophenyl)-3-methylpicolinamide;
N-(6-(2H-1,2,3-triazol-2-yl)-5-(trifluoromethyl)pyridin-3-yl)-2-(2-chloro-4-fluorophenyl)-4-(trifluoromethyl)pyrimidine-5-carboxamide;
N-(6-(2H-1,2,3-triazol-2-yl)-5-(trifluoromethyl)pyridin-3-yl)-2′-chloro-3,4′-difluoro-[1,1′-biphenyl]-4-carboxamide;
N-(6-(2H-1,2,3-triazol-2-yl)-5-(trifluoromethyl)pyridin-3-yl)-4-methyl-2-(2-(trifluoromethyl)phenyl)pyrimidine-5-carboxamide;
N-(6-(2H-1,2,3-triazol-2-yl)-5-(trifluoromethyl)pyridin-3-yl)-6-(2-chloro-4-fluorophenyl)-5-methylnicotinamide;
2-(2-chloro-4-fluorophenyl)-N-(5-chloro-6-(2H-1,2,3-triazol-2-yl)pyridin-3-yl)-4-methylpyrimidine-5-carboxamide;
N-(6-(2H-1,2,3-triazol-2-yl)-5-(trifluoromethyl)pyridin-3-yl)-6-(5-amino-2-chlorophenyl)-2-methoxynicotinamide;
N-(6-(2H-1,2,3-triazol-2-yl)-5-(trifluoromethyl)pyridin-3-yl)-6-methoxy-3′-methyl-[2,2′-bipyridine]-5-carboxamide;
N-(6-(2H-1,2,3-triazol-2-yl)-5-(trifluoromethyl)pyridin-3-yl)-6-(2-chloro-4-fluorophenyl)-4-methoxynicotinamide;
3-chloro-N-(5-chloro-6-(2H-1,2,3-triazol-2-yl)pyridin-3-yl)-2′-fluoro-6′-methyl-[1,1′-biphenyl]-4-carboxamide;
3,3′-dichloro-N-(5-chloro-6-(2H-1,2,3-triazol-2-yl)pyridin-3-yl)-2′-methyl-[1,1′-biphenyl]-4-carboxamide;
5-(3-chloro-2-methylphenyl)-N-(5-chloro-6-(2H-1,2,3-triazol-2-yl)pyridin-3-yl)-3-methylpicolinamide;
5-(2-chloro-4-fluorophenyl)-N-(5-chloro-6-(2H-1,2,3-triazol-2-yl)pyridin-3-yl)-3-methylpicolinamide;
N-(6-(2H-1,2,3-triazol-2-yl)-5-(trifluoromethyl)pyridin-3-yl)-2′-chloro-3-methyl-[1,1′-biphenyl]-4-carboxamide;
N-(6-(2H-1,2,3-triazol-2-yl)-5-(trifluoromethyl)pyridin-3-yl)-2′-amino-3-methyl-[1,1′-biphenyl]-4-carboxamide;
N-(6-(2H-1,2,3-triazol-2-yl)-5-(trifluoromethyl)pyridin-3-yl)-4′-chloro-3-methyl-[1,1′-biphenyl]-4-carboxamide;
N-(6-(2H-1,2,3-triazol-2-yl)-5-(trifluoromethyl)pyridin-3-yl)-2′-chloro-3,3′-dimethyl-[1,1′-biphenyl]-4-carboxamide;
N-(6-(2H-1,2,3-triazol-2-yl)-5-(trifluoromethyl)pyridin-3-yl)-2′,3′-dichloro-3-methyl-[1,1′-biphenyl]-4-carboxamide;
N-(6-(2H-1,2,3-triazol-2-yl)-5-(trifluoromethyl)pyridin-3-yl)-2′-fluoro-3,6′-dimethyl-[1,1′-biphenyl]-4-carboxamide;
N4′-(6-(2H-1,2,3-triazol-2-yl)-5-(trifluoromethyl)pyridin-3-yl)-3′-methyl-[1,1′-biphenyl]-2,4′-dicarboxamide;
2-chloro-N-(5-chloro-6-(2H-1,2,3-triazol-2-yl)pyridin-3-yl)-4-(3-methylpyridin-4-yl)benzamide;
2′,3-dichloro-N-(5-chloro-6-(2H-1,2,3-triazol-2-yl)pyridin-3-yl)-[1,1′-biphenyl]-4-carboxamide;
3-chloro-N-(5-chloro-6-(2H-1,2,3-triazol-2-yl)pyridin-3-yl)-2′-(trifluoromethoxy)-[1,1′-biphenyl]-4-carboxamide;
3-chloro-N-(5-chloro-6-(2H-1,2,3-triazol-2-yl)pyridin-3-yl)-3′-cyano-[1,1′-biphenyl]-4-carboxamide;
N-(5-chloro-6-(2H-1,2,3-triazol-2-yl)pyridin-3-yl)-5-(3-chlorophenyl)-3-methylpicolinamide;
N-(6-(2H-1,2,3-triazol-2-yl)-5-(trifluoromethyl)pyridin-3-yl)-3′-chloro-3-methyl-[1,1′-biphenyl]-4-carboxamide;
N-(6-(2H-1,2,3-triazol-2-yl)-5-(trifluoromethyl)pyridin-3-yl)-2′-cyano-3-methyl-[1,1′-biphenyl]-4-carboxamide;
N-(6-(2H-1,2,3-triazol-2-yl)-5-(trifluoromethyl)pyridin-3-yl)-2′-fluoro-3-methyl-4′-(trifluoromethyl)-[1,1′-biphenyl]-4-carboxamide;
N-(6-(2H-1,2,3-triazol-2-yl)-5-(trifluoromethyl)pyridin-3-yl)-2-methyl-4-(6-(trifluoromethyl)pyridin-3-yl)benzamide;
N-(6-(2H-1,2,3-triazol-2-yl)-5-(trifluoromethyl)pyridin-3-yl)-4-(furan-2-yl)-2-methylbenzamide;
N-(6-(2H-1,2,3-triazol-2-yl)-5-(trifluoromethyl)pyridin-3-yl)-4-(cyclopent-1-en-1-yl)-2-methylbenzamide;
N-(6-(2H-1,2,3-triazol-2-yl)-5-(trifluoromethyl)pyridin-3-yl)-5-(2-cyanophenyl)-3-methylpicolinamide;
N-(6-(2H-1,2,3-triazol-2-yl)-5-(trifluoromethyl)pyridin-3-yl)-2-methyl-4-(1-methyl-1H-pyrazol-5-yl)benzamide;
N-(6-(2H-1,2,3-triazol-2-yl)-5-(trifluoromethyl)pyridin-3-yl)-2′-chloro-3-cyano-[1,1′-biphenyl]-4-carboxamide;
N-(6-(2H-1,2,3-triazol-2-yl)-5-(trifluoromethyl)pyridin-3-yl)-4′-chloro-3′-cyano-3-methyl-[1,1′-biphenyl]-4-carboxamide;
N-(6-(2H-1,2,3-triazol-2-yl)-5-(trifluoromethyl)pyridin-3-yl)-2-methyl-4-(4-methylpyridin-3-yl)benzamide;
N-(6-(2H-1,2,3-triazol-2-yl)-5-(trifluoromethyl)pyridin-3-yl)-2-methyl-4-(1-oxoisoindolin-5-yl)benzamide;
N-(6-(2H-1,2,3-triazol-2-yl)-5-(trifluoromethyl)pyridin-3-yl)-2-methyl-4-(quinolin-4-yl)benzamide;
N4′-(6-(2H-1,2,3-triazol-2-yl)-5-(trifluoromethyl)pyridin-3-yl)-3′-methyl-[1,1′-biphenyl]-3,4′-dicarboxamide;
N-(6-(2H-1,2,3-triazol-2-yl)-5-(trifluoromethyl)pyridin-3-yl)-2′-methoxy-3-(trifluoromethyl)-[1,1′-biphenyl]-4-carboxamide;
N-(6-(2H-1,2,3-triazol-2-yl)-5-(trifluoromethyl)pyridin-3-yl)-2-methyl-4-(1H-pyrrolo[2,3-b]pyridin-5-yl)benzamide;
N-(6-(2H-1,2,3-triazol-2-yl)-5-(trifluoromethyl)pyridin-3-yl)-2′-methoxy-3-methyl-[1,1′-biphenyl]-4-carboxamide;
N-(6-(2H-1,2,3-triazol-2-yl)-5-(trifluoromethyl)pyridin-3-yl)-4-(6-fluoropyridin-3-yl)-2-methylbenzamide;
N-(6-(2H-1,2,3-triazol-2-yl)-5-(trifluoromethyl)pyridin-3-yl)-4-(6-cyanopyridin-3-yl)-2-methylbenzamide;
N-(6-(2H-1,2,3-triazol-2-yl)-5-(trifluoromethyl)pyridin-3-yl)-5-chloro-2-fluoro-2′-methoxy-[1,1′-biphenyl]-4-carboxamide;
N-(6-(2H-1,2,3-triazol-2-yl)-5-(trifluoromethyl)pyridin-3-yl)-2′-amino-3-(trifluoromethyl)-[1,1′-biphenyl]-4-carboxamide;
N-(6-(2H-1,2,3-triazol-2-yl)-5-(trifluoromethyl)pyridin-3-yl)-2′-amino-5-chloro-2-fluoro-[1,1′-biphenyl]-4-carboxamide;
N-(6-(2H-1,2,3-triazol-2-yl)-5-(trifluoromethyl)pyridin-3-yl)-2′-acetamido-3-(trifluoromethyl)-[1,1′-biphenyl]-4-carboxamide;
N-(6-(2H-1,2,3-triazol-2-yl)-5-(trifluoromethyl)pyridin-3-yl)-4-(4-chloropyridin-3-yl)-2-(trifluoromethyl)benzamide;
N-(6-(2H-1,2,3-triazol-2-yl)-5-(trifluoromethyl)pyridin-3-yl)-2′-(dimethylamino)-3-(trifluoromethyl)-[1,1′-biphenyl]-4-carboxamide;
N-(6-(2H-1,2,3-triazol-2-yl)-5-(trifluoromethyl)pyridin-3-yl)-2′-(dimethylamino)-3-methyl-[1,1′-biphenyl]-4-carboxamide;
N-(6-(2H-1,2,3-triazol-2-yl)-5-(trifluoromethyl)pyridin-3-yl)-3-chloro-2′-(dimethylamino)-[1,1′-biphenyl]-4-carboxamide;
N-(6-(2H-1,2,3-triazol-2-yl)-5-(trifluoromethyl)pyridin-3-yl)-3-cyano-2′-(dimethylamino)-[1,1′-biphenyl]-4-carboxamide;
N-(6-(2H-1,2,3-triazol-2-yl)-5-(trifluoromethyl)pyridin-3-yl)-5-chloro-2′-(dimethylamino)-2-fluoro-[1,1′-biphenyl]-4-carboxamide;
N-(5-chloro-6-(2H-1,2,3-triazol-2-yl)pyridin-3-yl)-4′,5-dimethyl-[3,3′-bipyridine]-6-carboxamide;
3′-chloro-N4′-(5-chloro-6-(2H-1,2,3-triazol-2-yl)pyridin-3-yl)-[1,1′-biphenyl]-3,4′-dicarboxamide;
2-chloro-N-(5-chloro-6-(2H-1,2,3-triazol-2-yl)pyridin-3-yl)-4-(1-methyl-1H-pyrazol-3-yl)benzamide;
2′-chloro-N-(5-chloro-6-(2H-1,2,3-triazol-2-yl)pyridin-3-yl)-4′-fluoro-[1,1′-biphenyl]-4-carboxamide;
2′-amino-N-(5-chloro-6-(2H-1,2,3-triazol-2-yl)pyridin-3-yl)-[1,1′-biphenyl]-4-carboxamide;
N-(6-(2H-1,2,3-triazol-2-yl)-5-(trifluoromethyl)pyridin-3-yl)-3-chloro-2′-methoxy-[1,1′-biphenyl]-4-carboxamide;
N-(6-(2H-1,2,3-triazol-2-yl)-5-(trifluoromethyl)pyridin-3-yl)-2′-amino-3-fluoro-[1,1′-biphenyl]-4-carboxamide;
N-(6-(2H-1,2,3-triazol-2-yl)-5-(trifluoromethyl)pyridin-3-yl)-2′-acetamido-3-fluoro-[1,1′-biphenyl]-4-carboxamide;
N-(6-(2H-1,2,3-triazol-2-yl)-5-(trifluoromethyl)pyridin-3-yl)-3-fluoro-2′-methoxy-[1,1′-biphenyl]-4-carboxamide;
N-(6-(2H-1,2,3-triazol-2-yl)-5-(trifluoromethyl)pyridin-3-yl)-3,4′-difluoro-2′-hydroxy-[1,1′-biphenyl]-4-carboxamide;
N-(6-(2H-1,2,3-triazol-2-yl)-5-(trifluoromethyl)pyridin-3-yl)-3-fluoro-2′-hydroxy-[1,1′-biphenyl]-4-carboxamide;
N-(6-(2H-1,2,3-triazol-2-yl)-5-(trifluoromethyl)pyridin-3-yl)-3-cyano-4′-fluoro-2′-hydroxy-[1,1′-biphenyl]-4-carboxamide;
N-(6-(2H-1,2,3-triazol-2-yl)-5-(trifluoromethyl)pyridin-3-yl)-3-cyano-2′-hydroxy-[1,1′-biphenyl]-4-carboxamide;
3-chloro-N-(5-chloro-6-(2H-1,2,3-triazol-2-yl)pyridin-3-yl)-4′-fluoro-2′-hydroxy-[1,1′-biphenyl]-4-carboxamide;
3-chloro-N-(5-chloro-6-(2H-1,2,3-triazol-2-yl)pyridin-3-yl)-4′-fluoro-[1,1′-biphenyl]-4-carboxamide;
2-chloro-N-(5-chloro-6-(2H-1,2,3-triazol-2-yl)pyridin-3-yl)-4-(3-chloropyridin-4-yl)benzamide;
3-chloro-N-(5-chloro-6-(2H-1,2,3-triazol-2-yl)pyridin-3-yl)-2′,4′-difluoro-[1,1′-biphenyl]-4-carboxamide;
2′-bromo-3-chloro-N-(5-chloro-6-(2H-1,2,3-triazol-2-yl)pyridin-3-yl)-4′-fluoro-[1,1′-biphenyl]-4-carboxamide;
3-chloro-N-(5-chloro-6-(2H-1,2,3-triazol-2-yl)pyridin-3-yl)-4′-fluoro-2′-(methylthio)-[1,1′-biphenyl]-4-carboxamide;
3-chloro-N-(5-chloro-6-(2H-1,2,3-triazol-2-yl)pyridin-3-yl)-4′-fluoro-2′-methoxy-[1,1′-biphenyl]-4-carboxamide;
N-(6-(2H-1,2,3-triazol-2-yl)-5-(trifluoromethyl)pyridin-3-yl)-2′,5-dichloro-2,4′-difluoro-[1,1′-biphenyl]-4-carboxamide;
N-(6-(2H-1,2,3-triazol-2-yl)-5-(trifluoromethyl)pyridin-3-yl)-2′,5-dichloro-2-fluoro-[1,1′-biphenyl]-4-carboxamide;
2′-amino-5-chloro-N-(5-chloro-6-(2H-1,2,3-triazol-2-yl)pyridin-3-yl)-2-fluoro-[1,1′-biphenyl]-4-carboxamide;
N-(6-(2H-1,2,3-triazol-2-yl)-5-(trifluoromethyl)pyridin-3-yl)-2′-amino-2-chloro-5-fluoro-[1,1′-biphenyl]-4-carboxamide;
N-(6-(2H-1,2,3-triazol-2-yl)-5-(trifluoromethyl)pyridin-3-yl)-2′-amino-2,5-dichloro-[1,1′-biphenyl]-4-carboxamide;
N-(6-(2H-1,2,3-triazol-2-yl)-5-(trifluoromethyl)pyridin-3-yl)-5-chloro-2,4′-difluoro-2′-methoxy-[1,1′-biphenyl]-4-carboxamide;
2′-amino-N-(5-chloro-6-(2H-1,2,3-triazol-2-yl)pyridin-3-yl)-2,4′-difluoro-5-methyl-[1,1′-biphenyl]-4-carboxamide;
2′-bromo-N-(5-chloro-6-(2H-1,2,3-triazol-2-yl)pyridin-3-yl)-2,4′-difluoro-5-methyl-[1,1′-biphenyl]-4-carboxamide;
2′-chloro-N-(5-chloro-6-(2H-1,2,3-triazol-2-yl)pyridin-3-yl)-2,4′-difluoro-5-methyl-[1,1′-biphenyl]-4-carboxamide;
N-(5-chloro-6-(2H-1,2,3-triazol-2-yl)pyridin-3-yl)-2,2′,4′-trifluoro-5-methyl-[1,1′-biphenyl]-4-carboxamide;
N-(5-chloro-6-(2H-1,2,3-triazol-2-yl)pyridin-3-yl)-2-fluoro-2′-methoxy-5-methyl-[1,1′-biphenyl]-4-carboxamide;
N-(5-chloro-6-(2H-1,2,3-triazol-2-yl)pyridin-3-yl)-2,4′-difluoro-2′-methoxy-5-methyl-[1,1′-biphenyl]-4-carboxamide;
2′-amino-2-chloro-N-(5-chloro-6-(2H-1,2,3-triazol-2-yl)pyridin-3-yl)-5-fluoro-[1,1′-biphenyl]-4-carboxamide;
2′-amino-2-chloro-N-(5-chloro-6-(2H-1,2,3-triazol-2-yl)pyridin-3-yl)-4′,5-difluoro-[1,1′-biphenyl]-4-carboxamide;
2′-amino-5-chloro-N-(5-chloro-6-(2H-1,2,3-triazol-2-yl)pyridin-3-yl)-2,4′-difluoro-[1,1′-biphenyl]-4-carboxamide;
N-(6-(2H-1,2,3-triazol-2-yl)-5-(trifluoromethyl)pyridin-3-yl)-2′-amino-5-chloro-2,4′-difluoro-[1,1′-biphenyl]-4-carboxamide;
N-(6-(2H-1,2,3-triazol-2-yl)-5-(trifluoromethyl)pyridin-3-yl)-2′-amino-2-chloro-4′,5-difluoro-[1,1′-biphenyl]-4-carboxamide;
4-(3-aminopyridin-4-yl)-5-chloro-N-(5-chloro-6-(2H-1,2,3-triazol-2-yl)pyridin-3-yl)-2-fluorobenzamide formate;
4-(3-aminopyridin-4-yl)-N-(5-chloro-6-(2H-1,2,3-triazol-2-yl)pyridin-3-yl)-5-fluoro-2-methylbenzamide;
N-(5-chloro-6-(2H-1,2,3-triazol-2-yl)pyridin-3-yl)-4-(3-chloropyridin-4-yl)-5-fluoro-2-methylbenzamide;
2-chloro-N-(5-chloro-6-(2H-1,2,3-triazol-2-yl)pyridin-3-yl)-4′,5-difluoro-2′-(methylamino)-[1,1′-biphenyl]-4-carboxamide;
N-(6-(2H-1,2,3-triazol-2-yl)-5-(trifluoromethyl)pyridin-3-yl)-4-(3-amino-5-fluoropyridin-2-yl)-2-chloro-5-fluorobenzamide;
N-(6-(2H-1,2,3-triazol-2-yl)-5-(trifluoromethyl)pyridin-3-yl)-3-chloro-2′-(difluoromethoxy)-4′-fluoro-[1,1′-biphenyl]-4-carboxamide;
4-(4-aminopyrimidin-5-yl)-2-chloro-N-(5-chloro-6-(2H-1,2,3-triazol-2-yl)pyridin-3-yl)-5-fluorobenzamide;
4-(3-aminopyridazin-4-yl)-2-chloro-N-(5-chloro-6-(2H-1,2,3-triazol-2-yl)pyridin-3-yl)-5-fluorobenzamide;
2-chloro-N-(5-chloro-6-(2H-1,2,3-triazol-2-yl)pyridin-3-yl)-4-(3-chloropyridazin-4-yl)-5-fluorobenzamide;
4-(6-amino-2-oxo-1,2-dihydropyrimidin-5-yl)-2-chloro-N-(5-chloro-6-(2H-1,2,3-triazol-2-yl)pyridin-3-yl)-5-fluorobenzamide;
2′-amino-N-(5-chloro-6-(2H-1,2,3-triazol-2-yl)pyridin-3-yl)-4′,5-difluoro-2-methoxy-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-methoxybenzamide;
2′-bromo-N-(5-chloro-6-(2H-1,2,3-triazol-2-yl)pyridin-3-yl)-4′,5-difluoro-2-methoxy-[1,1′-biphenyl]-4-carboxamide;
N-(6-(2H-1,2,3-triazol-2-yl)-5-(trifluoromethyl)pyridin-3-yl)-3-chloro-4′-fluoro-2′-iodo-[1,1′-biphenyl]-4-carboxamide;
N-(6-(2H-1,2,3-triazol-2-yl)-5-(trifluoromethyl)pyridin-3-yl)-2′,3-dibromo-4′-fluoro-[1,1′-biphenyl]-4-carboxamide;
N-(6-(2H-1,2,3-triazol-2-yl)-5-(trifluoromethyl)pyridin-3-yl)-2′-amino-3-bromo-4′-fluoro-[1,1′-biphenyl]-4-carboxamide;
N-(6-(2H-1,2,3-triazol-2-yl)-5-(trifluoromethyl)pyridin-3-yl)-5-chloro-2,4′-difluoro-2′-iodo-[1,1′-biphenyl]-4-carboxamide;
N-(6-(2H-1,2,3-triazol-2-yl)-5-(trifluoromethyl)pyridin-3-yl)-3-bromo-4′-fluoro-2′-iodo-[1,1′-biphenyl]-4-carboxamide;
N-(6-(2H-1,2,3-triazol-2-yl)-5-(trifluoromethyl)pyridin-3-yl)-2′-amino-5-bromo-2,4′-difluoro-[1,1′-biphenyl]-4-carboxamide;
N-(6-(2H-1,2,3-triazol-2-yl)-5-(trifluoromethyl)pyridin-3-yl)-2-(2-amino-4-fluorophenyl)-4-methylpyrimidine-5-carboxamide;
N-(6-(2H-1,2,3-triazol-2-yl)-5-(trifluoromethyl)pyridin-3-yl)-5-bromo-2,4′-difluoro-2′-iodo-[1,1′-biphenyl]-4-carboxamide;
N-(6-(2H-1,2,3-triazol-2-yl)-5-(trifluoromethyl)pyridin-3-yl)-2′-amino-4′-fluoro-3-iodo-[1,1′-biphenyl]-4-carboxamide;
N-(6-(2H-1,2,3-triazol-2-yl)-5-(trifluoromethyl)pyridin-3-yl)-2′-bromo-5-chloro-2,4′-difluoro-[1,1′-biphenyl]-4-carboxamide;
N-(6-(2H-1,2,3-triazol-2-yl)-5-(trifluoromethyl)pyridin-3-yl)-2,4′-difluoro-2′,5-diiodo-[1,1′-biphenyl]-4-carboxamide;
N-(6-(2H-1,2,3-triazol-2-yl)-5-(trifluoromethyl)pyridin-3-yl)-2′-amino-2,4′-difluoro-5-iodo-[1,1′-biphenyl]-4-carboxamide;
N-(6-(2H-1,2,3-triazol-2-yl)-5-(trifluoromethyl)pyridin-3-yl)-2′-amino-2,4′,5-trifluoro-[1,1′-biphenyl]-4-carboxamide;
N-(6-(2H-1,2,3-triazol-2-yl)-5-(trifluoromethyl)pyridin-3-yl)-2′-chloro-2,4′,5-trifluoro-[1,1′-biphenyl]-4-carboxamide;
N-(6-(2H-1,2,3-triazol-2-yl)-5-(trifluoromethyl)pyridin-3-yl)-4-(3-aminopyridin-4-yl)-2,5-difluorobenzamide;
N-(6-(2H-1,2,3-triazol-2-yl)-5-(trifluoromethyl)pyridin-3-yl)-2′-amino-2-chloro-4′,5-difluoro-[1,1′-biphenyl]-4-carboxamide;
2′-amino-2-bromo-N-(5-chloro-6-(2H-1,2,3-triazol-2-yl)pyridin-3-yl)-4′,5-difluoro-[1,1′-biphenyl]-4-carboxamide;
2′-bromo-2-chloro-N-(5-chloro-6-(2H-1,2,3-triazol-2-yl)pyridin-3-yl)-4′,5-difluoro-[1,1′-biphenyl]-4-carboxamide;
2′-amino-N-(5-chloro-6-(2H-1,2,3-triazol-2-yl)pyridin-3-yl)-4′,5-difluoro-2-iodo-[1,1′-biphenyl]-4-carboxamide;
2-chloro-N-(5-chloro-6-(2H-1,2,3-triazol-2-yl)pyridin-3-yl)-4′,5-difluoro-2′-iodo-[1,1′-biphenyl]-4-carboxamide;
N-(6-(2H-1,2,3-triazol-2-yl)-5-(trifluoromethyl)pyridin-3-yl)-4-(3-bromopyridin-4-yl)-2-chlorobenzamide;
N-(6-(2H-1,2,3-triazol-2-yl)-5-(trifluoromethyl)pyridin-3-yl)-4-(3-aminopyridin-4-yl)-2-bromo-5-fluorobenzamide;
N-(6-(2H-1,2,3-triazol-2-yl)-5-(trifluoromethyl)pyridin-3-yl)-4-(3-bromopyridin-4-yl)-2-chloro-5-fluorobenzamide;
N-(6-(2H-1,2,3-triazol-2-yl)-5-(trifluoromethyl)pyridin-3-yl)-4-(3-aminopyridin-4-yl)-5-fluoro-2-iodobenzamide;
N-(6-(2H-1,2,3-triazol-2-yl)-5-(trifluoromethyl)pyridin-3-yl)-2-chloro-5-fluoro-4-(3-iodopyridin-4-yl)benzamide;
N-(6-(2H-1,2,3-triazol-2-yl)-5-(trifluoromethyl)pyridin-3-yl)-4-(3-aminopyridin-4-yl)-5-fluoro-2-methylbenzamide;
N-(6-(2H-1,2,3-triazol-2-yl)-5-(trifluoromethyl)pyridin-3-yl)-4-(3-aminopyridin-4-yl)-2-chloro-5-fluorobenzamide;
N-(6-(2H-1,2,3-triazol-2-yl)-5-(trifluoromethyl)pyridin-3-yl)-2-chloro-5-fluoro-4-(3-methoxypyridin-4-yl)benzamide;
N-(6-(2H-1,2,3-triazol-2-yl)-5-(trifluoromethyl)pyridin-3-yl)-4-(3-chloropyridin-4-yl)-2,5-difluorobenzamide;
N-(6-(2H-1,2,3-triazol-2-yl)-5-(trifluoromethyl)pyridin-3-yl)-2-chloro-4-(3-chloropyridin-4-yl)-5-fluorobenzamide;
2,2′-dibromo-N-(5-chloro-6-(2H-1,2,3-triazol-2-yl)pyridin-3-yl)-4′,5-difluoro-[1,1′-biphenyl]-4-carboxamide;
4-(3-aminopyridin-4-yl)-5-chloro-N-(5-chloro-6-(2H-1,2,3-triazol-2-yl)pyridin-3-yl)-2-fluorobenzamide;
4-(3-bromopyridin-4-yl)-5-chloro-N-(5-chloro-6-(2H-1,2,3-triazol-2-yl)pyridin-3-yl)-2-fluorobenzamide;
5-chloro-N-(5-chloro-6-(2H-1,2,3-triazol-2-yl)pyridin-3-yl)-4-(3-chloropyridin-4-yl)-2-fluorobenzamide;
(S)-4-(3-aminopyridin-4-yl)-N-(5-chloro-6-(2H-1,2,3-triazol-2-yl)pyridin-3-yl)-2-fluoro-5-(1-methoxyethyl)benzamide;
(S)-4-(3-amino-5-fluoropyridin-2-yl)-N-(5-chloro-6-(2H-1,2,3-triazol-2-yl)pyridin-3-yl)-2-fluoro-5-(1-methoxyethyl)benzamide;
2′-amino-N-(5-chloro-6-(2H-1,2,3-triazol-2-yl)pyridin-3-yl)-4′,5-difluoro-2-isopropoxy-[1,1′-biphenyl]-4-carboxamide;
2′-bromo-N-(5-chloro-6-(2H-1,2,3-triazol-2-yl)pyridin-3-yl)-4′,5-difluoro-2-isopropoxy-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-methylbenzamide;
4-(3-aminopyridin-4-yl)-N-(5-chloro-6-(2H-1,2,3-triazol-2-yl)pyridin-3-yl)-2-fluoro-5-isopropylbenzamide;
2′-amino-N-(5-chloro-6-(2H-1,2,3-triazol-2-yl)pyridin-3-yl)-4′,5-difluoro-2-isobutyl-[1,1′-biphenyl]-4-carboxamide;
2′-bromo-N-(5-chloro-6-(2H-1,2,3-triazol-2-yl)pyridin-3-yl)-4′,5-difluoro-2-isobutyl-[1,1′-biphenyl]-4-carboxamide;
4-(3-aminopyridin-4-yl)-N-(5-chloro-6-(2H-1,2,3-triazol-2-yl)pyridin-3-yl)-5-cyclopropyl-2-fluorobenzamide;
4-(3-aminopyridin-4-yl)-N-(5-chloro-6-(2H-1,2,3-triazol-2-yl)pyridin-3-yl)-2-fluoro-5-isopropoxybenzamide;
2′-amino-N-(5-chloro-6-(2H-1,2,3-triazol-2-yl)pyridin-3-yl)-2-cyclopropyl-4′,5-difluoro-[1,1′-biphenyl]-4-carboxamide;
2′-bromo-N-(5-chloro-6-(2H-1,2,3-triazol-2-yl)pyridin-3-yl)-2-cyclopropyl-4′,5-difluoro-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-(tetrahydro-2H-pyran-4-yl)benzamide;
4-(3-aminopyridin-4-yl)-N-(5-chloro-6-(2H-1,2,3-triazol-2-yl)pyridin-3-yl)-5-ethynyl-2-fluorobenzamide;
2′-amino-N-(5-chloro-6-(2H-1,2,3-triazol-2-yl)pyridin-3-yl)-2-ethynyl-4′,5-difluoro-[1,1′-biphenyl]-4-carboxamide;
2′-bromo-N-(5-chloro-6-(2H-1,2,3-triazol-2-yl)pyridin-3-yl)-2-ethynyl-4′,5-difluoro-[1,1′-biphenyl]-4-carboxamide;
4-(3-aminopyridin-4-yl)-5-(tert-butyl)-N-(5-chloro-6-(2H-1,2,3-triazol-2-yl)pyridin-3-yl)-2-fluorobenzamide;
4-(3-aminopyridin-4-yl)-N-(5-chloro-6-(2H-1,2,3-triazol-2-yl)pyridin-3-yl)-2-fluoro-5-isobutylbenzamide;
2′-bromo-N-(5-chloro-6-(2H-1,2,3-triazol-2-yl)pyridin-3-yl)-4′,5-difluoro-2-(oxetan-3-yl)-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-(oxetan-3-yl)benzamide;
4-(3-aminopyridin-4-yl)-N-(5-chloro-6-(2H-1,2,3-triazol-2-yl)pyridin-3-yl)-5-ethyl-2-fluorobenzamide;
2′-amino-N-(5-chloro-6-(2H-1,2,3-triazol-2-yl)pyridin-3-yl)-4′,5-difluoro-2-isopropyl-[1,1′-biphenyl]-4-carboxamide;
2′-bromo-N-(5-chloro-6-(2H-1,2,3-triazol-2-yl)pyridin-3-yl)-4′,5-difluoro-2-isopropyl-[1,1′-biphenyl]-4-carboxamide;
5-chloro-N-(5-chloro-6-(2H-1,2,3-triazol-2-yl)pyridin-3-yl)-2′-(difluoromethyl)-2,4′-difluoro-[1,1′-biphenyl]-4-carboxamide;
5-chloro-N-(5-chloro-6-(2H-1,2,3-triazol-2-yl)pyridin-3-yl)-2′-(difluoromethoxy)-2,4′-difluoro-[1,1′-biphenyl]-4-carboxamide;
2-chloro-N-(5-chloro-6-(2H-1,2,3-triazol-2-yl)pyridin-3-yl)-5-fluoro-4-(6-oxo-1,6-dihydropyridin-3-yl)benzamide;
2-chloro-N-(5-chloro-6-(2H-1,2,3-triazol-2-yl)pyridin-3-yl)-5-fluoro-4-(1-methyl-6-oxo-1,6-dihydropyridin-3-yl)benzamide;
2-chloro-N-(5-chloro-6-(2H-1,2,3-triazol-2-yl)pyridin-3-yl)-5-fluoro-4-(5-fluoro-1-methyl-6-oxo-1,6-dihydropyridin-3-yl)benzamide;
N-(5-chloro-6-(2H-1,2,3-triazol-2-yl)pyridin-3-yl)-2,4′-difluoro-2′-hydroxy-5-methyl-[1,1′-biphenyl]-4-carboxamide;
N-(5-chloro-6-(2H-1,2,3-triazol-2-yl)pyridin-3-yl)-5-fluoro-4-(3-hydroxypyridin-4-yl)-2-methylbenzamide;
4-(5-aminopyrimidin-4-yl)-N-(5-chloro-6-(2H-1,2,3-triazol-2-yl)pyridin-3-yl)-5-fluoro-2-methylbenzamide;
2-chloro-N-(5-chloro-6-(2H-1,2,3-triazol-2-yl)pyridin-3-yl)-5-fluoro-4-(5-fluoro-3-methylpyridin-2-yl)benzamide;
5-chloro-N-(5-chloro-6-(2H-1,2,3-triazol-2-yl)pyridin-3-yl)-2′-(difluoromethyl)-2,4′-difluoro-[1,1′-biphenyl]-4-carboxamide;
2-chloro-N-(5-chloro-6-(2H-1,2,3-triazol-2-yl)pyridin-3-yl)-5-fluoro-4-(6-oxo-1,6-dihydropyridin-3-yl)benzamide;
N-(5-chloro-6-(2H-1,2,3-triazol-2-yl)pyridin-3-yl)-2,4′-difluoro-2′-hydroxy-5-methyl-[1,1′-biphenyl]-4-carboxamide;
5-chloro-N-(5-chloro-6-(2H-1,2,3-triazol-2-yl)pyridin-3-yl)-2′-cyano-2,4′-difluoro-[1,1′-biphenyl]-4-carboxamide;
5-chloro-N-(5-chloro-6-(2H-1,2,3-triazol-2-yl)pyridin-3-yl)-2′-(difluoromethoxy)-2,4′-difluoro-[1,1′-biphenyl]-4-carboxamide;
2-chloro-N-(5-chloro-6-(2H-1,2,3-triazol-2-yl)pyridin-3-yl)-5-fluoro-4-(1-methyl-6-oxo-1,6-dihydropyridin-3-yl)benzamide;
N-(5-chloro-6-(2H-1,2,3-triazol-2-yl)pyridin-3-yl)-5-fluoro-4-(3-hydroxypyridin-4-yl)-2-methylbenzamide;
2-chloro-N-(5-chloro-6-(2H-1,2,3-triazol-2-yl)pyridin-3-yl)-4-(3-cyanopyridin-4-yl)-5-fluorobenzamide;
N-(6-(2H-1,2,3-triazol-2-yl)-5-(trifluoromethyl)pyridin-3-yl)-2′-amino-5-chloro-4′-fluoro-2-(trifluoromethyl)-[1,1′-biphenyl]-4-carboxamide;
N-(6-(2H-1,2,3-triazol-2-yl)-5-(trifluoromethyl)pyridin-3-yl)-2,2′,3,4′,5,6-hexafluoro-[1,1′-biphenyl]-4-carboxamide;
2′-chloro-N-(5-chloro-6-(2H-1,2,3-triazol-2-yl)pyridin-3-yl)-2,3,4′,5,6-pentafluoro-[1,1′-biphenyl]-4-carboxamide;
N-(6-(2H-1,2,3-triazol-2-yl)-5-(trifluoromethyl)pyridin-3-yl)-2′,5-dichloro-4′-fluoro-2-(trifluoromethyl)-[1,1′-biphenyl]-4-carboxamide;
N-(6-(2H-1,2,3-triazol-2-yl)-5-(trifluoromethyl)pyridin-3-yl)-5-chloro-2′,4′-difluoro-2-(trifluoromethyl)-[1,1′-biphenyl]-4-carboxamide;
2′-amino-5-chloro-N-(5-chloro-6-(2H-1,2,3-triazol-2-yl)pyridin-3-yl)-2-(trifluoromethyl)-[1,1′-biphenyl]-4-carboxamide;
5-chloro-N-(5-chloro-6-(2H-1,2,3-triazol-2-yl)pyridin-3-yl)-2,2′,4′-trifluoro-[1,1′-biphenyl]-4-carboxamide;
2′-bromo-5-chloro-N-(5-chloro-6-(2H-1,2,3-triazol-2-yl)pyridin-3-yl)-2,4′-difluoro-[1,1′-biphenyl]-4-carboxamide;
2′-amino-5-chloro-N-(5-chloro-6-(2H-1,2,3-triazol-2-yl)pyridin-3-yl)-4′-fluoro-2-(trifluoromethyl)-[1,1′-biphenyl]-4-carboxamide;
2′-amino-5-chloro-N-(5-chloro-6-(2H-1,2,3-triazol-2-yl)pyridin-3-yl)-2-cyclopropyl-4′-fluoro-[1,1′-biphenyl]-4-carboxamide;
5-chloro-N-(5-chloro-6-(2H-1,2,3-triazol-2-yl)pyridin-3-yl)-2′-(difluoromethyl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(trifluoromethyl)-[1,1′-biphenyl]-4-carboxamide;
5-chloro-N-(5-chloro-6-(2H-1,2,3-triazol-2-yl)pyridin-3-yl)-2′-cyclopropyl-2,4′-difluoro-[1,1′-biphenyl]-4-carboxamide;
2′-amino-5-chloro-N-(5-chloro-6-(2H-1,2,3-triazol-2-yl)pyridin-3-yl)-4′-fluoro-2-methyl-[1,1′-biphenyl]-4-carboxamide;
4-(3-aminopyridin-4-yl)-2-chloro-N-(5-chloro-6-(2H-1,2,3-triazol-2-yl)pyridin-3-yl)-5-(trifluoromethyl)benzamide;
2-chloro-N-(5-chloro-6-(2H-1,2,3-triazol-2-yl)pyridin-3-yl)-4-(3-chloropyridin-4-yl)-5-(trifluoromethyl)benzamide;
4-(3-bromopyridin-4-yl)-2-chloro-N-(5-chloro-6-(2H-1,2,3-triazol-2-yl)pyridin-3-yl)-5-(trifluoromethyl)benzamide;
2′-amino-5-chloro-N-(5-chloro-6-(2H-1,2,3-triazol-2-yl)pyridin-3-yl)-2-ethynyl-4′-fluoro-[1,1′-biphenyl]-4-carboxamide;
2′,5-dichloro-N-(5-chloro-6-(2H-1,2,3-triazol-2-yl)pyridin-3-yl)-4′-fluoro-2-(trifluoromethyl)-[1,1′-biphenyl]-4-carboxamide;
2′-bromo-5-chloro-N-(5-chloro-6-(2H-1,2,3-triazol-2-yl)pyridin-3-yl)-4′-fluoro-2-(trifluoromethyl)-[1,1′-biphenyl]-4-carboxamide;
5-chloro-N-(5-chloro-6-(2H-1,2,3-triazol-2-yl)pyridin-3-yl)-4′-fluoro-2′-methyl-2-(trifluoromethyl)-[1,1′-biphenyl]-4-carboxamide;
2′-amino-2,5-dichloro-N-(5-chloro-6-(2H-1,2,3-triazol-2-yl)pyridin-3-yl)-4′-fluoro-[1,1′-biphenyl]-4-carboxamide;
2′,5-dichloro-2,4′-difluoro-N-(6-methyl-5-oxo-5,6-dihydro-1,6-naphthyridin-3-yl)-[1,1′-biphenyl]-4-carboxamide;
2′,5-dichloro-N-(7-chloro-2-methylbenzo[d]oxazol-5-yl)-2,4′-difluoro-[1,1′-biphenyl]-4-carboxamide;
2′,5-dichloro-N-(8-chloro-3-oxo-3,4-dihydro-2H-benzo[b][1,4]oxazin-6-yl)-2,4′-difluoro-[1,1′-biphenyl]-4-carboxamide;
2′,5-dichloro-2,4′-difluoro-N-(1-methyl-1H-pyrazolo[4,3-b]pyridin-6-yl)-[1,1′-biphenyl]-4-carboxamide;
2′,5-dichloro-2,4′-difluoro-N-(2-methyl-2H-pyrazolo[4,3-b]pyridin-6-yl)-[1,1′-biphenyl]-4-carboxamide;
2′,5-dichloro-2,4′-difluoro-N-(1-methyl-1H-pyrazolo[3,4-b]pyridin-5-yl)-[1,1′-biphenyl]-4-carboxamide;
2′,5-dichloro-N-(2-chloropyrazolo[1,5-a]pyrimidin-6-yl)-2,4′-difluoro-[1,1′-biphenyl]-4-carboxamide;
2′,5-dichloro-2,4′-difluoro-N-(2-methylimidazo[1,2-b]pyridazin-7-yl)-[1,1′-biphenyl]-4-carboxamide;
2′,5-dichloro-N-(2-chlorothiazolo[5,4-b]pyridin-6-yl)-2,4′-difluoro-[1,1′-biphenyl]-4-carboxamide;
2′,5-dichloro-N-(6-chloro-1,5-naphthyridin-3-yl)-2,4′-difluoro-[1,1′-biphenyl]-4-carboxamide;
2′,5-dichloro-2,4′-difluoro-N-(2-(trifluoromethyl)pyridin-4-yl)-[1,1′-biphenyl]-4-carboxamide;
2′,5-dichloro-N-(2-cyanopyridin-4-yl)-2,4′-difluoro-[1,1′-biphenyl]-4-carboxamide;
2′,5-dichloro-N-(2-chloropyridin-4-yl)-2,4′-difluoro-[1,1′-biphenyl]-4-carboxamide;
2′,5-dichloro-N-(5-chloro-6-methoxypyridin-3-yl)-2,4′-difluoro-[1,1′-biphenyl]-4-carboxamide;
2′,5-dichloro-N-(6-cyano-5-(trifluoromethyl)pyridin-3-yl)-2,4′-difluoro-[1,1′-biphenyl]-4-carboxamide;
2′,5-dichloro-N-(6-cyano-5-fluoropyridin-3-yl)-2,4′-difluoro-[1,1′-biphenyl]-4-carboxamide;
2′,5-dichloro-2,4′-difluoro-N-(2-methyl-6-(trifluoromethyl)pyridin-4-yl)-[1,1′-biphenyl]-4-carboxamide;
2′,5-dichloro-N-(5-chloro-6-(1-methyl-1H-pyrazol-4-yl)pyridin-3-yl)-2,4′-difluoro-[1,1′-biphenyl]-4-carboxamide;
2′,5-dichloro-N-(5-chloro-6-(3-methyl-1H-1,2,4-triazol-1-yl)pyridin-3-yl)-2,4′-difluoro-[1,1′-biphenyl]-4-carboxamide;
2′,5-dichloro-N-(5-chloro-6-(oxazol-2-yl)pyridin-3-yl)-2,4′-difluoro-[1,1′-biphenyl]-4-carboxamide;
2′,5-dichloro-N-(5-chloro-6-(thiazol-2-yl)pyridin-3-yl)-2,4′-difluoro-[1,1′-biphenyl]-4-carboxamide;
2′,5-dichloro-N-(5-chloro-6-(1H-pyrazol-1-yl)pyridin-3-yl)-2,4′-difluoro-[1,1′-biphenyl]-4-carboxamide;
2′,5-dichloro-N-(5-cyano-6-(2H-1,2,3-triazol-2-yl)pyridin-3-yl)-2,4′-difluoro-[1,1′-biphenyl]-4-carboxamide;
2′,5-dichloro-N-(5-cyano-6-cyclopropoxypyridin-3-yl)-2,4′-difluoro-[1,1′-biphenyl]-4-carboxamide;
2′,5-dichloro-N-(5-cyano-6-(difluoromethoxy)pyridin-3-yl)-2,4′-difluoro-[1,1′-biphenyl]-4-carboxamide;
4-chloro-6-(2-chloro-4-fluorophenyl)-N-(5-chloro-6-(2H-1,2,3-triazol-2-yl)pyridin-3-yl)nicotinamide;
6-(2-amino-4-fluorophenyl)-4-chloro-N-(5-chloro-6-(2H-1,2,3-triazol-2-yl)pyridin-3-yl)nicotinamide;
2-(2-amino-4-fluorophenyl)-4-chloro-N-(5-chloro-6-(2H-1,2,3-triazol-2-yl)pyridin-3-yl)pyrimidine-5-carboxamide;
6-(2-amino-4-fluorophenyl)-2-chloro-N-(5-chloro-6-(2H-1,2,3-triazol-2-yl)pyridin-3-yl)nicotinamide;
4-chloro-6-(2-chloro-4-fluorophenyl)-N-(5-chloro-6-(2H-1,2,3-triazol-2-yl)pyridin-3-yl)pyridazine-3-carboxamide;
3-chloro-5-(2-chloro-4-fluorophenyl)-N-(5-chloro-6-(2H-1,2,3-triazol-2-yl)pyridin-3-yl)pyrazine-2-carboxamide;
N-(6-(2H-1,2,3-triazol-2-yl)-5-(trifluoromethyl)pyridin-3-yl)-4-(3-bromopyridin-4-yl)-2-chloro-5-(trifluoromethyl)benzamide;
2-amino-2′,5-dichloro-N-(5-chloro-6-(2H-1,2,3-triazol-2-yl)pyridin-3-yl)-4′-fluoro-[1,1′-biphenyl]-4-carboxamide;
2-acetamido-2′,5-dichloro-N-(5-chloro-6-(2H-1,2,3-triazol-2-yl)pyridin-3-yl)-4′-fluoro-[1,1′-biphenyl]-4-carboxamide;
ethyl(2′,5-dichloro-4-((5-chloro-6-(2H-1,2,3-triazol-2-yl)pyridin-3-yl)aminoformyl)-4′-fluoro-[1,1′-biphenyl]-2-yl)aminoformate;
2′,5-dichloro-N-(5-chloro-6-(2H-1,2,3-triazol-2-yl)pyridin-3-yl)-4′-fluoro-2-(isopropylamino)-[1,1′-biphenyl]-4-carboxamide;
N-(6-(2H-1,2,3-triazol-2-yl)-5-(trifluoromethyl)pyridin-3-yl)-5-chloro-2,2′,4′-trifluoro-[1,1′-biphenyl]-4-carboxamide;
5-chloro-N-(5-chloro-6-(2H-1,2,3-triazol-2-yl)pyridin-3-yl)-2,2′,4′-trifluoro-[1,1′-biphenyl]-4-carboxamide;
N-(5-chloro-6-(2H-1,2,3-triazol-2-yl)pyridin-3-yl)-2,2′,4′,5-tetrafluoro-[1,1′-biphenyl]-4-carboxamide;
N-(6-(2H-1,2,3-triazol-2-yl)-5-(trifluoromethyl)pyridin-3-yl)-2,2′,4′,5-tetrafluoro-[1,1′-biphenyl]-4-carboxamide;
4-(3-bromopyridin-4-yl)-2-chloro-N-(5-chloro-6-(2H-1,2,3-triazol-2-yl)pyridin-3-yl)-5-fluorobenzamide;
4-(2-amino-6-fluoropyridin-3-yl)-N-(5-chloro-6-(2H-1,2,3-triazol-2-yl)pyridin-3-yl)-2-Fluoro-5-isopropylbenzamide;
5-chloro-N-(5-chloro-6-(2H-1,2,3-triazol-2-yl)pyridin-3-yl)-2′-(cyclopropylamino)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(methylthio)-[1,1′-biphenyl]-4-carboxamide;
5-chloro-N-(5-chloro-6-(2H-1,2,3-triazol-2-yl)pyridin-3-yl)-2,4′-difluoro-2′-(methylsulfonyl)-[1,1′-biphenyl]-4-carboxamide;
5-chloro-N-(5-chloro-6-(2H-1,2,3-triazol-2-yl)pyridin-3-yl)-2,4′-difluoro-2′-ureido-[1,1′-biphenyl]-4-carboxamide;
2′-acetamido-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(hydroxymethyl)-[1,1′-biphenyl]-4-carboxamide;
4-(3-amino-5-ethynylpyridin-4-yl)-2-chloro-N-(5-chloro-6-(2H-1,2,3-triazol-2-yl)pyridin-3-yl)-5-fluorobenzamide;
5-chloro-N-(5-chloro-6-(2H-1,2,3-triazol-2-yl)pyridin-3-yl)-2,4′-difluoro-2′-(methylsulfinyl)-[1,1′-biphenyl]-4-carboxamide;
5-chloro-N-(5-chloro-6-(2H-1,2,3-triazol-2-yl)pyridin-3-yl)-2,4′-difluoro-2′-((methylsulfonyl)methyl)-[1,1′-biphenyl]-4-carboxamide;
ethyl (5′-chloro-4′-((5-chloro-6-(2H-1,2,3-triazol-2-yl)pyridin-3-yl)carbamoyl)-2′,4-difluoro-[1,1′-biphenyl]-2-yl)carbamate;
5-chloro-N-(5-chloro-6-(2H-1,2,3-triazol-2-yl)pyridin-3-yl)-2,4′-difluoro-2′-vinyl-[1,1′-biphenyl]-4-carboxamide;
5-chloro-N-(5-chloro-6-(2H-1,2,3-triazol-2-yl)pyridin-3-yl)-2,4′-difluoro-2′-(1-hydroxyethyl)-[1,1′-biphenyl]-4-carboxamide
5-chloro-N-(5-chloro-6-(2H-1,2,3-triazol-2-yl)pyridin-3-yl)-2,4′-difluoro-2′-(1-methoxyethyl)-[1,1′-biphenyl]-4-carboxamide;
5-chloro-N-(5-chloro-6-(2H-1,2,3-triazol-2-yl)pyridin-3-yl)-2,4′-difluoro-2′-((2-(methylamino)ethyl)amino)-[1,1′-biphenyl]-4-carboxamide;
5-chloro-N-(5-chloro-6-(2H-1,2,3-triazol-2-yl)pyridin-3-yl)-2,4′-difluoro-2′-((2-hydroxyethyl)amino)-[1,1′-biphenyl]-4-carboxamide;
5-chloro-N-(5-chloro-6-(2H-1,2,3-triazol-2-yl)pyridin-3-yl)-2,4′-difluoro-2′-(2-hydroxyethoxy)-[1,1′-biphenyl]-4-carboxamide;
5-chloro-N-(5-chloro-6-(2H-1,2,3-triazol-2-yl)pyridin-3-yl)-2,4′-difluoro-2′-(hydroxyamino)-[1,1′-biphenyl]-4-carboxamide;
5-chloro-N-(5-chloro-6-(2H-1,2,3-triazol-2-yl)pyridin-3-yl)-2,4′-difluoro-2′-(2-hydroxypropan-2-yl)-[1,1′-biphenyl]-4-carboxamide;
5′-chloro-4′-((5-chloro-6-(2H-1,2,3-triazol-2-yl)pyridin-3-yl)carbamoyl)-2′,4-difluoro-[1,1′-biphenyl]-2-yl ethyl carbonate;
5-chloro-N-(5-chloro-6-(2H-1,2,3-triazol-2-yl)pyridin-3-yl)-2′-((2-(dimethylamino)ethyl)amino)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-((2-methoxyethyl)amino)-[1,1′-biphenyl]-4-carboxamide;
5-chloro-N-(5-chloro-6-(2H-1,2,3-triazol-2-yl)pyridin-3-yl)-2,4′-difluoro-2′-(2-methoxyethoxy)-[1,1′-biphenyl]-4-carboxamide;
5-chloro-N-(5-chloro-6-(2H-1,2,3-triazol-2-yl)pyridin-3-yl)-2,4′-difluoro-2′-(prop-2-yn-1-ylamino)-[1,1′-biphenyl]-4-carboxamide;
5-chloro-N-(5-chloro-6-(2H-1,2,3-triazol-2-yl)pyridin-3-yl)-2′-((cyanomethyl)amino)-2,4′-difluoro-[1,1′-biphenyl]-4-carboxamide;
2′-(aminomethyl)-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(prop-2-yn-1-yl)-[1,1′-biphenyl]-4-carboxamide;
2′-acrylamido-5-chloro-N-(5-chloro-6-(2H-1,2,3-triazol-2-yl)pyridin-3-yl)-2,4′-difluoro-[1,1′-biphenyl]-4-carboxamide;
5′-chloro-N4′-(5-chloro-6-(2H-1,2,3-triazol-2-yl)pyridin-3-yl)-2′,4-difluoro-[1,1′-biphenyl]-2,4′-dicarboxamide;
5-chloro-N-(5-chloro-6-(2H-1,2,3-triazol-2-yl)pyridin-3-yl)-2,4′-difluoro-2′-(prop-2-yn-1-yloxy)-[1,1′-biphenyl]-4-carboxamide;
5-chloro-N-(5-chloro-6-(2H-1,2,3-triazol-2-yl)pyridin-3-yl)-2′-(cyanomethoxy)-2,4′-difluoro-[1,1′-biphenyl]-4-carboxamide;
2′-(1-aminoethyl)-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2′-(cyanomethyl)-2,4′-difluoro-[1,1′-biphenyl]-4-carboxamide;
2′-amino-5-chloro-N-(5-chloro-6-(2H-1,2,3-triazol-2-yl)pyridin-3-yl)-6′-ethynyl-2,4′-difluoro-[1,1′-biphenyl]-4-carboxamide;
cyclopropyl 5′-chloro-4′-((5-chloro-6-(2H-1,2,3-triazol-2-yl)pyridin-3-yl)carbamoyl)-2′,4-difluoro-[1,1′-biphenyl]-2-carboxylate;
2′,5-dichloro-N-(5-chloro-6-(2H-1,2,3-triazol-2-yl)pyridin-3-yl)-6′-ethynyl-2,4′-difluoro-[1,1′-biphenyl]-4-carboxamide;
2′-(2-aminopropan-2-yl)-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(methoxymethyl)-[1,1′-biphenyl]-4-carboxamide;
5′-chloro-4′-((5-chloro-6-(2H-1,2,3-triazol-2-yl)pyridin-3-yl)carbamoyl)-2′,4-difluoro-[1,1′-biphenyl]-2-yl carbamate;
2′,5-dichloro-N-(5-chloro-6-(2H-1,2,3-triazol-2-yl)pyridin-3-yl)-2,4′-difluoro-6′-(methoxymethyl)-[1,1′-biphenyl]-4-carboxamide;
N-(6-(2H-1,2,3-triazol-2-yl)-5-(trifluoromethyl)pyridin-3-yl)-5-chloro-2,4′-difluoro-2′-vinyl-[1,1′-biphenyl]-4-carboxamide;
N-(6-(2H-1,2,3-triazol-2-yl)-5-(trifluoromethyl)pyridin-3-yl)-5-chloro-2′-ethynyl-2,4′-difluoro-[1,1′-biphenyl]-4-carboxamide;
2-chloro-N-(5-chloro-6-(2H-1,2,3-triazol-2-yl)pyridin-3-yl)-4-(2-ethynyl-6-fluoropyridin-3-yl)-5-fluorobenzamide;
2-chloro-N-(5-chloro-6-(2H-1,2,3-triazol-2-yl)pyridin-3-yl)-4-(3-ethynylpyridin-4-yl)-5-fluorobenzamide;
5-chloro-2′-ethynyl-2,4′-difluoro-N-(2-(trifluoromethyl)pyridin-4-yl)-[1,1′-biphenyl]-4-carboxamide;
5-chloro-N-(8-chloro-3-oxo-3,4-dihydro-2H-benzo[b][1,4]oxazin-6-yl)-2′-ethynyl-2,4′-difluoro-[1,1′-biphenyl]-4-carboxamide;
5-chloro-N-(6-cyano-5-(trifluoromethyl)pyridin-3-yl)-2′-ethynyl-2,4′-difluoro-[1,1′-biphenyl]-4-carboxamide;
N-(6-(2H-1,2,3-triazol-2-yl)-5-(trifluoromethyl)pyridin-3-yl)-2-chloro-4-(2-ethynyl-6-fluoropyridin-3-yl)-5-fluorobenzamide;
2-chloro-4-(3-ethynylpyridin-4-yl)-5-fluoro-N-(6-((tetrahydrofuran-3-yl)amino)-5-(trifluoromethyl)pyridin-3-yl)benzamide;
2-chloro-4-(3-ethynyl-5-fluoropyridin-2-yl)-5-fluoro-N-(2-(trifluoromethyl)pyridin-4-yl)benzamide;
2-chloro-N-(8-chloro-3-oxo-3,4-dihydro-2H-benzo[b][1,4]oxazin-6-yl)-4-(3-ethynyl-5-fluoropyridin-2-yl)-5-fluorobenzamide;
2-chloro-N-(6-cyano-5-(trifluoromethyl)pyridin-3-yl)-4-(3-ethynyl-5-fluoropyridin-2-yl)-5-fluorobenzamide;
N-(6-(2H-1,2,3-triazol-2-yl)-5-(trifluoromethyl)pyridin-3-yl)-2-chloro-4-(3-ethynyl-5-fluoropyridin-2-yl)-5-fluorobenzamide;
5-chloro-N-(5-cyano-6-(trifluoromethoxy)pyridin-3-yl)-2′-ethynyl-2,4′-difluoro-[1,1′-biphenyl]-4-carboxamide;
5-chloro-N-(5-chloro-6-(difluoromethoxy)pyridin-3-yl)-2′-ethynyl-2,4′-difluoro-[1,1′-biphenyl]-4-carboxamide;
5-chloro-N-(5-cyano-6-((1-methylpiperidin-4-yl)oxy)pyridin-3-yl)-2′-ethynyl-2,4′-difluoro-[1,1′-biphenyl]-4-carboxamide;
N-(6-(2H-1,2,3-triazol-2-yl)-5-(trifluoromethyl)pyridin-3-yl)-2-chloro-4-(3-ethynylpyridin-4-yl)-5-fluorobenzamide;
N-(5-chloro-6-(2H-1,2,3-triazol-2-yl)pyridin-3-yl)-2,3,4′,5,6-pentafluoro-2′-propiolamido-[1,1′-biphenyl]-4-carboxamide;
N-(5-chloro-6-(2H-1,2,3-triazol-2-yl)pyridin-3-yl)-2,3,4′,5,6-pentafluoro-2′-propiolamido-[1,1′-biphenyl]-4-carboxamide;
N-(5-chloro-6-(2H-1,2,3-triazol-2-yl)pyridin-3-yl)-4-(3-ethynylpyridin-4-yl)-2,3,5,6-tetrafluorobenzamide;
2-chloro-N-(3-chloro-4-(2-methoxyethoxy)phenyl)-4-(3-ethynylpyridin-4-yl)-5-fluorobenzamide;
2-chloro-N-(5-chloro-6-(2H-1,2,3-triazol-2-yl)pyridin-3-yl)-4-(3-ethynyl-5-fluoropyridin-2-yl)-5-fluorobenzamide;
6-chloro-N-(5-chloro-6-(2H-1,2,3-triazol-2-yl)pyridin-3-yl)-3′-ethynyl-[2,4′-bipyridine]-5-carboxamide;
2-chloro-N-(5-chloro-6-(2H-1,2,3-triazol-2-yl)pyridin-3-yl)-5-fluoro-4-(3-propiolamidopyridin-4-yl)benzamide;
2-chloro-N-(3-chloro-4-methoxyphenyl)-4-(3-ethynylpyridin-4-yl)-5-fluorobenzamide;
2-chloro-4-(3-ethynyl-5-fluoropyridin-2-yl)-5-fluoro-N-(6-((tetrahydrofuran-3-yl)amino)-5-(trifluoromethyl)pyridin-3-yl)benzamide;
N-(5-chloro-6-(2H-1,2,3-triazol-2-yl)pyridin-3-yl)-4-(3-ethynyl-5-fluoropyridin-2-yl)-2,3,5,6-tetrafluorobenzamide;
N-(5-chloro-6-(2H-1,2,3-triazol-2-yl)pyridin-3-yl)-2,3,5,6-tetrafluoro-4-(5-fluoro-3-propiolamidopyridin-2-yl)benzamide;
4-(3-acrylamidopyridin-4-yl)-2-chloro-N-(5-chloro-6-(2H-1,2,3-triazol-2-yl)pyridin-3-yl)-5-fluorobenzamide;
N-(3-chloro-4-(methylsulfonyl)phenyl)-4-(3-ethynylpyridin-4-yl)-2,3,5,6-tetrafluorobenzamide;
N-(3-chloro-4-((trifluoromethyl)thio)phenyl)-4-(3-ethynylpyridin-4-yl)-2,3,5,6-tetrafluorobenzamide;
N-(3-chloro-4-((2-(dimethylamino)-2-oxoethyl)thio)phenyl)-4-(3-ethynylpyridin-4-yl)-2,3,5,6-tetrafluorobenzamide;
N-(6-(2H-1,2,3-triazol-2-yl)-5-(trifluoromethyl)pyridin-3-yl)-2-chloro-5-fluoro-4-(3-vinylpyridin-4-yl)benzamide;
2′-amino-5-chloro-2,4′-difluoro-N-(6-(2-methoxyethoxy)-5-(trifluoromethyl)pyridin-3-yl)-[1,1′-biphenyl]-4-carboxamide;
2′-amino-5-chloro-N-(6-(cyclopropylamino)-5-(trifluoromethyl)pyridin-3-yl)-2,4′-difluoro-[1,1′-biphenyl]-4-carboxamide;
2′-amino-5-chloro-N-(6-(cyanomethoxy)-5-(trifluoromethyl)pyridin-3-yl)-2,4′-difluoro-[1,1′-biphenyl]-4-carboxamide;
2′-amino-5-chloro-N-(6-((cyanomethyl)amino)-5-(trifluoromethyl)pyridin-3-yl)-2,4′-difluoro-[1,1′-biphenyl]-4-carboxamide;
2′-amino-5-chloro-2,4′-difluoro-N-(6-(methoxymethyl)-5-(trifluoromethyl)pyridin-3-yl)-[1,1′-biphenyl]-4-carboxamide;
2′-amino-5-chloro-2,4′-difluoro-N-(6-((2-methoxyethyl)amino)-5-(trifluoromethyl)pyridin-3-yl)-[1,1′-biphenyl]-4-carboxamide;
methyl (4-(2′-amino-5-chloro-2,4′-difluoro-[1,1′-biphenyl]-4-carboxamido)-2-(trifluoromethyl)phenyl)carbamate;
N-(4-acetamido-3-(trifluoromethyl)phenyl)-2′-amino-5-chloro-2,4′-difluoro-[1,1′-biphenyl]-4-carboxamide;
2′-amino-5-chloro-2,4′-difluoro-N-(3-(trifluoromethyl)-4-ureidophenyl)-[1,1′-biphenyl]-4-carboxamide;
2′-amino-5-chloro-2,4′-difluoro-N-(4-(methylsulfonamido)-3-(trifluoromethyl)phenyl)-[1,1′-biphenyl]-4-carboxamide;
2′-amino-5-chloro-2,4′-difluoro-N-(6-(hydroxymethyl)-5-(trifluoromethyl)pyridin-3-yl)-[1,1′-biphenyl]-4-carboxamide;
2′-amino-5-chloro-N-(6-(cyanomethyl)-5-(trifluoromethyl)pyridin-3-yl)-2,4′-difluoro-[1,1′-biphenyl]-4-carboxamide;
2′-amino-5-chloro-2,4′-difluoro-N-(4-(methylthio)-3-(trifluoromethyl)phenyl)-[1,1′-biphenyl]-4-carboxamide;
2′-amino-5-chloro-2,4′-difluoro-N-(4-(methylsulfonyl)-3-(trifluoromethyl)phenyl)-[1,1′-biphenyl]-4-carboxamide;
2′-amino-5-chloro-2,4′-difluoro-N-(6-((tetrahydrofuran-3-yl)oxy)-5-(trifluoromethyl)pyridin-3-yl)-[1,1′-biphenyl]-4-carboxamide;
2′-amino-5-chloro-2,4′-difluoro-N-(6-(2-hydroxypropan-2-yl)-5-(trifluoromethyl)pyridin-3-yl)-[1,1′-biphenyl]-4-carboxamide;
5-(2′-amino-5-chloro-2,4′-difluoro-[1,1′-biphenyl]-4-carboxamido)-3-(trifluoromethyl)picolinamide;
2′-amino-5-chloro-N-(6-ethynyl-5-(trifluoromethyl)pyridin-3-yl)-2,4′-difluoro-[1,1′-biphenyl]-4-carboxamide;
2′-amino-5-chloro-2,4′-difluoro-N-(6-((tetrahydrofuran-3-yl)amino)-5-(trifluoromethyl)pyridin-3-yl)-[1,1′-biphenyl]-4-carboxamide;
2′-amino-5-chloro-N-(4-(cyclopropylsulfonyl)-3-(trifluoromethyl)phenyl)-2,4′-difluoro-[1,1′-biphenyl]-4-carboxamide;
2′-amino-5-chloro-2,4′-difluoro-N-(6-(pyrrolidine-1-carbonyl)-5-(trifluoromethyl)pyridin-3-yl)-[1,1′-biphenyl]-4-carboxamide;
2′-amino-5-chloro-N-(4-(N,N-dimethylsulfamoyl)-3-(trifluoromethyl)phenyl)-2,4′-difluoro-[1,1′-biphenyl]-4-carboxamide;
N-(4-(2-azaspiro[3.3]heptane-2-carbonyl)-3-(trifluoromethyl)phenyl)-2′-amino-5-chloro-2,4′-difluoro-[1,1′-biphenyl]-4-carboxamide;
2-chloro-N-(5-chloro-6-(1-oxo-2-oxa-7-azaspiro[4.4]nonan-7-yl)pyridin-3-yl)-4-(3-ethynylpyridin-4-yl)-5-fluorobenzamide;
2-chloro-N-(5-chloro-6-(2-oxa-7-azaspiro[4.4]nonan-7-yl)pyridin-3-yl)-4-(3-ethynylpyridin-4-yl)-5-fluorobenzamide;
2-chloro-N-(5-chloro-6-((1R,2S,4S)-2-(3-methylisoxazol-5-yl)-7-azabicyclo[2.2.1]heptan-7-yl)pyridin-3-yl)-4-(3-ethynylpyridin-4-yl)-5-fluorobenzamide;
2-chloro-N-(5-chloro-6-(1-cyano-7-azabicyclo[2.2.1]heptan-7-yl)pyridin-3-yl)-4-(3-ethynylpyridin-4-yl)-5-fluorobenzamide;
N-(6-(7-azabicyclo[2.2.1]heptan-7-yl)-5-chloropyridin-3-yl)-2-chloro-4-(3-ethynylpyridin-4-yl)-5-fluorobenzamide;
2-chloro-N-(5-chloro-6-(4-oxohexahydrocyclopenta[c]pyrrol-2(1H)-yl)pyridin-3-yl)-4-(3-ethynylpyridin-4-yl)-5-fluorobenzamide;
2-chloro-N-(5-chloro-6-((3aR,6aS)-5-oxohexahydrocyclopenta[c]pyrrol-2(1H)-yl)pyridin-3-yl)-4-(3-ethynylpyridin-4-yl)-5-fluorobenzamide;
2-chloro-N-(5-chloro-6-((3aR,6aS)-5,5-difluorohexahydrocyclopenta[c]pyrrol-2(1H)-yl)pyridin-3-yl)-4-(3-ethynylpyridin-4-yl)-5-fluorobenzamide;
2-chloro-N-(5-chloro-6-((2-oxopyrrolidin-1-yl)amino)pyridin-3-yl)-4-(3-ethynylpyridin-4-yl)-5-fluorobenzamide;
2-chloro-N-(5-chloro-6-(3-oxopyrazolidin-1-yl)pyridin-3-yl)-4-(3-ethynylpyridin-4-yl)-5-fluorobenzamide;
N-(6-(1H-benzo[d]imidazol-2-yl)-5-chloropyridin-3-yl)-2-chloro-4-(3-ethynylpyridin-4-yl)-5-fluorobenzamide;
2-chloro-N-(5-chloro-6-(4,5,6,7-tetrahydro-2H-[1,2,3]triazolo[4,5-c]pyridin-2-yl)pyridin-3-yl)-4-(3-ethynylpyridin-4-yl)-5-fluorobenzamide;
2-chloro-N-(5-chloro-6-(4,5-dicyano-2H-1,2,3-triazol-2-yl)pyridin-3-yl)-4-(3-ethynylpyridin-4-yl)-5-fluorobenzamide;
2-chloro-N-(5-chloro-6-(4,5,6,7-tetrahydro-2H-indazol-2-yl)pyridin-3-yl)-4-(3-ethynylpyridin-4-yl)-5-fluorobenzamide;
2-chloro-N-(5-chloro-6-(5,6-dihydrocyclopenta[c]pyrazol-2(4H)-yl)pyridin-3-yl)-4-(3-ethynylpyridin-4-yl)-5-fluorobenzamide;
2-chloro-N-(5-chloro-6-(3-(trifluoromethyl)-5,6-dihydrocyclopenta[c]pyrazol-2(4H)-yl)pyridin-3-yl)-4-(3-ethynylpyridin-4-yl)-5-fluorobenzamide;
ethyl 2-((3-chloro-5-(2-chloro-4-(3-ethynylpyridin-4-yl)-5-fluorobenzamido)pyridin-2-yl)amino)-2,4,5,6-tetrahydrocyclopenta[d][1,2,3]triazole-4-carboxylate;
N-(6-((2H-benzo[d][1,2,3]triazol-2-yl)amino)-5-chloropyridin-3-yl)-2-chloro-4-(3-ethynylpyridin-4-yl)-5-fluorobenzamide;
2-chloro-N-(5-chloro-6-(2-(trifluoromethyl)-5,6-dihydro-[1,2,4]triazolo[1,5-a]pyrazin-7(8H)-yl)pyridin-3-yl)-4-(3-ethynylpyridin-4-yl)-5-fluorobenzamide;
2-chloro-N-(5-chloro-6-(3-(trifluoromethyl)-5,6-dihydro-[1,2,4]triazolo[4,3-a]pyrazin-7(8H)-yl)pyridin-3-yl)-4-(3-ethynylpyridin-4-yl)-5-fluorobenzamide;
1-(3-chloro-5-(2-chloro-4-(3-ethynylpyridin-4-yl)-5-fluorobenzamido)pyridin-2-yl)-4,5,6,7-tetrahydro-1H-benzo[d]imidazole-5-carboxylic acid;
2-chloro-N-(2-(3,4-dichlorophenyl)benzo[d]oxazol-5-yl)-4-(3-ethynylpyridin-4-yl)-5-fluorobenzamide;
(S)-2-chloro-N-(5-chloro-6-(2-cyano-4,4-difluoropyrrolidine-1-carbonyl)pyridin-3-yl)-4-(3-ethynylpyridin-4-yl)-5-fluorobenzamide;
2-chloro-N-(5-chloro-6-((3aR,6aS)-hexahydro-1H-furo[3,4-c]pyrrole-5-carbonyl)pyridin-3-yl)-4-(3-ethynylpyridin-4-yl)-5-fluorobenzamide;
2-chloro-N-(5-chloro-6-(pyrrolidine-1-carbonyl)pyridin-3-yl)-4-(3-ethynylpyridin-4-yl)-5-fluorobenzamide;
2-chloro-N-(5-chloro-6-(4,4-difluoropiperidine-1-carbonyl)pyridin-3-yl)-4-(3-ethynylpyridin-4-yl)-5-fluorobenzamide;
2-chloro-N-(5-chloro-6-(6,6-difluoro-3-azabicyclo[3.1.0]hexane-3-carbonyl)pyridin-3-yl)-4-(3-ethynylpyridin-4-yl)-5-fluorobenzamide;
2-chloro-N-(5-chloro-6-(6,6-difluoro-3-azabicyclo[3.1.0]hexan-3-yl)pyridin-3-yl)-4-(3-ethynylpyridin-4-yl)-5-fluorobenzamide;
2-chloro-N-(5-chloro-6-(3-(4,4-difluoropiperidin-1-yl)azetidin-1-yl)pyridin-3-yl)-4-(3-ethynylpyridin-4-yl)-5-fluorobenzamide;
2-chloro-N-(5-chloro-6-(3-(4,4-difluoropiperidin-1-yl)azetidine-1-carbonyl)pyridin-3-yl)-4-(3-ethynylpyridin-4-yl)-5-fluorobenzamide;
4,4′-dichloro-N-(5-chloro-6-(2H-1,2,3-triazol-2-yl)pyridin-3-yl)-6-(3-ethynylpyridin-4-yl)-[1,1′-biphenyl]-3-carboxamide;
2-amino-4″,5′-dichloro-N-(5-chloro-6-(2H-1,2,3-triazol-2-yl)pyridin-3-yl)-4-fluoro-[1,1′:2′,1″-terphenyl]-4′-carboxamide;
N-(6-(2H-1,2,3-triazol-2-yl)-5-(trifluoromethyl)pyridin-3-yl)-5′-methyl-[1,1′:2′,1″-terphenyl]-4′-carboxamide;
2-chloro-N-(5-chloro-6-(2H-1,2,3-triazol-2-yl)pyridin-3-yl)-5-ethynyl-4-(3-ethynylpyridin-4-yl)benzamide;
N-(5-chloro-6-(2H-1,2,3-triazol-2-yl)pyridin-3-yl)-5-ethynyl-4-(3-ethynylpyridin-4-yl)-2-methylbenzamide;
N-(5-chloro-6-(2H-1,2,3-triazol-2-yl)pyridin-3-yl)-5-cyclopropyl-4-(3-ethynylpyridin-4-yl)-2-methylbenzamide;
4-(3-amino-5-fluoropyridin-2-yl)-N-(5-chloro-6-(2H-1,2,3-triazol-2-yl)pyridin-3-yl)-5-cyclopropyl-2-methylbenzamide;
2-chloro-N-(5-chloro-6-(2H-1,2,3-triazol-2-yl)pyridin-3-yl)-5-cyclopropyl-4-(3-ethynylpyridin-4-yl)benzamide;
4-(3-amino-5-fluoropyridin-2-yl)-2-chloro-N-(5-chloro-6-(2H-1,2,3-triazol-2-yl)pyridin-3-yl)-5-cyclopropylbenzamide;
2′-amino-N-(5-chloro-6-(2H-1,2,3-triazol-2-yl)pyridin-3-yl)-2-ethynyl-4′-fluoro-5-methyl-[1,1′-biphenyl]-4-carboxamide;
N-(6-(2H-1,2,3-triazol-2-yl)-5-(trifluoromethyl)pyridin-3-yl)-4-(3-amino-5-ethynylpyridin-4-yl)-2-chloro-5-fluorobenzamide;
4-(3-amino-5-ethynylpyridin-4-yl)-2-chloro-N-(5-chloro-6-(2H-1,2,3-triazol-2-yl)pyridin-3-yl)-5-fluorobenzamide;
2′-amino-5-chloro-N-(5-chloro-6-(2H-1,2,3-triazol-2-yl)pyridin-3-yl)-6′-ethynyl-2,4′-difluoro-[1,1′-biphenyl]-4-carboxamide;
N-(6-(2H-1,2,3-triazol-2-yl)-5-(trifluoromethyl)pyridin-3-yl)-2′,5-dichloro-6′-ethynyl-2-fluoro-[1,1′-biphenyl]-4-carboxamide;
2′-chloro-N-(5-chloro-6-(2H-1,2,3-triazol-2-yl)pyridin-3-yl)-2-ethynyl-4′-fluoro-5-methyl-[1,1′-biphenyl]-4-carboxamide;
N-(5-chloro-6-(2H-1,2,3-triazol-2-yl)pyridin-3-yl)-2,2′-diethynyl-4′-fluoro-5-methyl-[1,1′-biphenyl]-4-carboxamide;
4-(2-amino-6-fluoropyridin-3-yl)-N-(5-chloro-6-(2H-1,2,3-triazol-2-yl)pyridin-3-yl)-5-ethynyl-2-methylbenzamide;
N-(5-chloro-6-(2H-1,2,3-triazol-2-yl)pyridin-3-yl)-5-ethynyl-4-(3-methoxypyridin-4-yl)-2-methylbenzamide;
2-amino-5′-chloro-N-(5-chloro-6-(2H-1,2,3-triazol-2-yl)pyridin-3-yl)-4-fluoro-2″,3″,4″,5″-tetrahydro-[1,1′:2′,1″-terphenyl]-4′-carboxamide;
2-chloro-N-(5-chloro-6-(2H-1,2,3-triazol-2-yl)pyridin-3-yl)-4-fluoro-5′-methyl-[1,1′:2′,1″-terphenyl]-4′-carboxamide;
2′-amino-N-(5-chloro-6-(2H-1,2,3-triazol-2-yl)pyridin-3-yl)-2-(cyclopent-1-en-1-yl)-4′-fluoro-5-methyl-[1,1′-biphenyl]-4-carboxamide;
2-amino-N-(5-chloro-6-(2H-1,2,3-triazol-2-yl)pyridin-3-yl)-4-fluoro-5′-methyl-[1,1′:2′,1″-terphenyl]-4′-carboxamide;
N-(5-chloro-6-(2H-1,2,3-triazol-2-yl)pyridin-3-yl)-2-ethynyl-4-fluoro-5′-methyl-[1,1′:2′,1″-terphenyl]-4′-carboxamide;
2′-amino-N-(5-chloro-6-(2H-1,2,3-triazol-2-yl)pyridin-3-yl)-4′-fluoro-5-methyl-2-(prop-1-yn-1-yl)-[1,1′-biphenyl]-4-carboxamide;
2′-amino-5-chloro-N-(5-chloro-6-(2H-1,2,3-triazol-2-yl)pyridin-3-yl)-4′-fluoro-2-vinyl-[1,1′-biphenyl]-4-carboxamide;
2′-amino-N-(5-chloro-6-(2H-1,2,3-triazol-2-yl)pyridin-3-yl)-4′-fluoro-5-methyl-2-vinyl-[1,1′-biphenyl]-4-carboxamide;
3-chloro-5-(2-chloro-4-(3-ethynylpyridin-4-yl)-5-fluorobenzamido)-N-methylpicolinamide;
3-chloro-5-(2-chloro-4-(3-ethynylpyridin-4-yl)-5-fluorobenzamido)-N-(cyanomethyl)picolinamide;
3-chloro-5-(2-chloro-4-(3-ethynylpyridin-4-yl)-5-fluorobenzamido)-N-ethylpicolinamide;
3-chloro-5-(2-chloro-4-(3-ethynylpyridin-4-yl)-5-fluorobenzamido)-N-(2,2,2-trifluoroethyl)picolinamide;
3-chloro-5-(2-chloro-4-(3-ethynylpyridin-4-yl)-5-fluorobenzamido)-N-propylpicolinamide;
3-chloro-5-(2-chloro-4-(3-ethynylpyridin-4-yl)-5-fluorobenzamido)-N-(2-(trifluoromethoxy)ethyl)picolinamide;
3-chloro-5-(2-chloro-4-(3-ethynylpyridin-4-yl)-5-fluorobenzamido)-N-phenethylpicolinamide;
3-chloro-5-(2-chloro-4-(3-ethynylpyridin-4-yl)-5-fluorobenzamido)-N-(furan-3-ylmethyl)picolinamide;
3-chloro-5-(2-chloro-4-(3-ethynylpyridin-4-yl)-5-fluorobenzamido)-N-neopentylpicolinamide;
3-chloro-5-(2-chloro-4-(3-ethynylpyridin-4-yl)-5-fluorobenzamido)-N-(2-(diethylamino)-2-oxoethyl)picolinamide;
5-(2′-amino-5-chloro-2,4′-difluoro-[1,1′-biphenyl]-4-carboxamido)-3-chloro-N-((1-methylcyclopropyl)methyl)picolinamide;
5-(2′-amino-5-chloro-2,4′-difluoro-[1,1′-biphenyl]-4-carboxamido)-3-chloro-N-((1,1-dioxidotetrahydro-2H-thiopyran-4-yl)methyl)picolinamide;
5-(2′-amino-5-chloro-2,4′-difluoro-[1,1′-biphenyl]-4-carboxamido)-N-butyl-3-chloropicolinamide;
5-(2′-amino-5-chloro-2,4′-difluoro-[1,1′-biphenyl]-4-carboxamido)-3-chloro-N-(prop-2-yn-1-yl)picolinamide;
N-(6-(2H-1,2,3-triazol-2-yl)-5-(trifluoromethyl)pyridin-3-yl)-4-(3-bromo-5-nitropyridin-4-yl)-2-chloro-5-fluorobenzamide;
5-(2′-amino-5-chloro-2,4′-difluoro-[1,1′-biphenyl]-4-carboxamido)-3-chloro-N-(cyanomethyl)picolinamide;
5-(2′-amino-5-chloro-2,4′-difluoro-6′-methyl-[1,1′-biphenyl]-4-carboxamido)-3-chloro-N-(cyanomethyl)picolinamide;
5-(2′-amino-5-chloro-2,4′-difluoro-[1,1′-biphenyl]-4-carboxamido)-3-chloro-N-(2,2,2-trifluoroethyl)picolinamide;
5-(2′-amino-5-chloro-2,4′-difluoro-[1,1′-biphenyl]-4-carboxamido)-3-chloro-N-ethylpicolinamide;
2-chloro-N-(5-chloro-6-(2H-1,2,3-triazol-2-yl)pyridin-3-yl)-4-(3-cyclopropylpyridin-4-yl)-5-fluorobenzamide;
5-(2′-amino-5-chloro-2,4′-difluoro-[1,1′-biphenyl]-4-carboxamido)-3-chloro-N-neopentylpicolinamide;
5-(2′-amino-5-chloro-2,4′-difluoro-[1,1′-biphenyl]-4-carboxamido)-3-chloro-N-(furan-3-ylmethyl)picolinamide;
5-(2′-amino-5-chloro-2,4′-difluoro-[1,1′-biphenyl]-4-carboxamido)-3-chloro-N-propylpicolinamide;
5-(2′-amino-5-chloro-2,4′-difluoro-[1,1′-biphenyl]-4-carboxamido)-3-chloro-N-(2-methoxyethyl)picolinamide;
5-(2′-amino-5-chloro-2,4′-difluoro-[1,1′-biphenyl]-4-carboxamido)-3-chloro-N-(1-cyanoethyl)picolinamide;
5-(2′-amino-5-chloro-2,4′-difluoro-[1,1′-biphenyl]-4-carboxamido)-3-chloro-N-((3,3-difluorocyclobutyl)methyl)picolinamide;
N-((1,3,4-oxadiazol-2-yl)methyl)-5-(2′-amino-5-chloro-2,4′-difluoro-[1,1′-biphenyl]-4-carboxamido)-3-chloropicolinamide;
5-(2′-amino-5-chloro-2,4′-difluoro-[1,1′-biphenyl]-4-carboxamido)-3-chloro-N-(2-(dimethylamino)ethyl)picolinamide;
5-(2′-amino-5-chloro-2,4′-difluoro-[1,1′-biphenyl]-4-carboxamido)-3-chloro-N-(cyclobutylmethyl)picolinamide;
5-(2′-amino-5-chloro-2,4′-difluoro-[1,1′-biphenyl]-4-carboxamido)-3-chloro-N-((1-methyl-1H-pyrazol-3-yl)methyl)picolinamide;
5-(4-(2-amino-6-fluoropyridin-3-yl)-2-chloro-5-fluorobenzamido)-3-chloro-N-(cyanomethyl)picolinamide;
5-(2′-amino-5-chloro-2,4′-difluoro-[1,1′-biphenyl]-4-carboxamido)-3-chloro-N-(2,2-difluoro-2λ3-ethyl)picolinamide;
5-(2′-amino-5-chloro-2,4′-difluoro-[1,1′-biphenyl]-4-carboxamido)-3-chloro-N-(2,2,3,3,4,4,4-heptafluorobutyl)picolinamide;
5-(2′-amino-5-chloro-2,4′-difluoro-[1,1′-biphenyl]-4-carboxamido)-3-chloro-N-(2-(trifluoromethoxy)ethyl)picolinamide;
2,2′-diamino-5-chloro-N-(5-chloro-6-(2H-1,2,3-triazol-2-yl)pyridin-3-yl)-4′-fluoro-[1,1′-biphenyl]-4-carboxamide;
5-(2′-amino-5-chloro-2,4′-difluoro-[1,1′-biphenyl]-4-carboxamido)-3-chloro-N-(2-fluoroethyl)picolinamide;
5-(2′-amino-5-chloro-2,4′-difluoro-[1,1′-biphenyl]-4-carboxamido)-3-chloro-N-((5-methyl-1,3,4-oxadiazol-2-yl)methyl)picolinamide;
N-((1H-1,2,4-triazol-3-yl)methyl)-5-(2′-amino-5-chloro-2,4′-difluoro-[1,1′-biphenyl]-4-carboxamido)-3-chloropicolinamide;
5-(2′-amino-5-chloro-2,4′-difluoro-[1,1′-biphenyl]-4-carboxamido)-3-chloro-N-(2-(ethylthio)ethyl)picolinamide;
N-(2-amino-2-oxoethyl)-5-(2′-amino-5-chloro-2,4′-difluoro-[1,1′-biphenyl]-4-carboxamido)-3-chloropicolinamide;
5-(2′-amino-5-chloro-2,4′-difluoro-[1,1′-biphenyl]-4-carboxamido)-3-chloro-N-((1-cyanocyclopropyl)methyl)picolinamide;
5-(2′-amino-5-chloro-2,4′-difluoro-[1,1′-biphenyl]-4-carboxamido)-3-chloro-N-(2,2,3,3,3-pentafluoropropyl)picolinamide;
5-(2′-amino-5-chloro-2,4′-difluoro-[1,1′-biphenyl]-4-carboxamido)-3-chloro-N-((1-(trifluoromethyl)cyclopropyl)methyl)picolinamide;
5-(2′-amino-5-chloro-2,4′-difluoro-[1,1′-biphenyl]-4-carboxamido)-3-chloro-N-(2-(methylsulfonyl)ethyl)picolinamide;
5-(4-(3-amino-5-ethynylpyridin-4-yl)-2-chloro-5-fluorobenzamido)-3-chloro-N-(2,2,2-trifluoroethyl)picolinamide;
5-(4-(3-amino-5-ethynylpyridin-4-yl)-2-chloro-5-fluorobenzamido)-3-chloro-N-(2,2-difluoroethyl)picolinamide;
5-(4-(3-amino-5-ethynylpyridin-4-yl)-2-chloro-5-fluorobenzamido)-3-chloro-N-(2-fluoroethyl)picolinamide;
5-(4-(3-amino-5-ethynylpyridin-4-yl)-2-chloro-5-fluorobenzamido)-3-chloro-N-((1,1-dioxidotetrahydro-2H-thiopyran-4-yl)methyl)picolinamide;
5-(4-(3-amino-5-ethynylpyridin-4-yl)-2-chloro-5-fluorobenzamido)-3-chloro-N-(furan-3-ylmethyl)picolinamide;
N-(6-(2H-1,2,3-triazol-2-yl)-5-(trifluoromethyl)pyridin-3-yl)-4-(3-amino-5-cyclopropylpyridin-4-yl)-2-chloro-5-fluorobenzamide;
5-(4-(3-amino-5-ethynylpyridin-4-yl)-2-chloro-5-fluorobenzamido)-3-chloro-N-(1-cyanoethyl)picolinamide;
5-(4-(3-amino-5-ethynylpyridin-4-yl)-2-chloro-5-fluorobenzamido)-3-chloro-N-(cyclopropylmethyl)picolinamide;
5-(4-(3-amino-5-ethynylpyridin-4-yl)-2-chloro-5-fluorobenzamido)-3-chloro-N-neopentylpicolinamide;
5-(4-(3-amino-5-ethynylpyridin-4-yl)-2-chloro-5-fluorobenzamido)-3-chloro-N-ethylpicolinamide;
5-(4-(3-amino-5-ethynylpyridin-4-yl)-2-chloro-5-fluorobenzamido)-3-chloro-N-(1-cyanocyclopropyl)picolinamide;
5-(4-(3-amino-5-ethynylpyridin-4-yl)-2-chloro-5-fluorobenzamido)-3-chloro-N-((1-cyanocyclopropyl)methyl)picolinamide;
5-(4-(3-amino-5-ethynylpyridin-4-yl)-2-chloro-5-fluorobenzamido)-3-chloro-N-(2-(methylsulfonyl)ethyl)picolinamide;
5-(4-(3-amino-5-ethynylpyridin-4-yl)-2-chloro-5-fluorobenzamido)-3-chloro-N-(2-(ethylthio)ethyl)picolinamide;
5-(4-(3-amino-5-ethynylpyridin-4-yl)-2-chloro-5-fluorobenzamido)-3-chloro-N-((5-methyl-1,3,4-oxadiazol-2-yl)methyl)picolinamide;
4-(3-amino-5-ethynyl-2-fluoropyridin-4-yl)-2-chloro-N-(5-chloro-6-(2H-1,2,3-triazol-2-yl)pyridin-3-yl)-5-fluorobenzamide;
4-(5-amino-3-ethynyl-2-fluoropyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-fluoropyridin-2-yl)-2-chloro-5-ethynylbenzamide;
N-(6-(2H-1,2,3-triazol-2-yl)-5-(trifluoromethyl)pyridin-3-yl)-4-(3-amino-5-(2-methylprop-1-en-1-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(1-ethoxyvinyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(1-ethoxyethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(1-hydroxyethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(2-cyanoethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(2-ethoxyethyl)pyridin-4-yl)-2-chloro-5-fluorobenzamide;
N-(6-(2H-1,2,3-triazol-2-yl)-5-(trifluoromethyl)pyridin-3-yl)-4-(3-acetyl-5-aminopyridin-4-yl)-2-chloro-5-fluorobenzamide;
N-(6-(2H-1,2,3-triazol-2-yl)-5-(trifluoromethyl)pyridin-3-yl)-4-(3-amino-5-(3,3,3-trifluoroprop-1-en-2-yl)pyridin-4-yl)-2-chloro-5-fluorobenzamide;
(E)-N-(6-(2H-1,2,3-triazol-2-yl)-5-(trifluoromethyl)pyridin-3-yl)-4-(3-amino-5-(3-hydroxy-3-methylbut-1-en-1-yl)pyridin-4-yl)-2-chloro-5-fluorobenzamide;
(E)-N-(6-(2H-1,2,3-triazol-2-yl)-5-(trifluoromethyl)pyridin-3-yl)-4-(3-amino-5-(2-cyclopropylvinyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(2-cyclopropylethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(3-hydroxy-3-methylbutyl)pyridin-4-yl)-2-chloro-5-fluorobenzamide;
(E)-N-(6-(2H-1,2,3-triazol-2-yl)-5-(trifluoromethyl)pyridin-3-yl)-4-(3-amino-5-(3-methoxyprop-1-en-1-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(3-methoxypropyl)pyridin-4-yl)-2-chloro-5-fluorobenzamide;
5-(4-(3-amino-5-ethynylpyridin-4-yl)-2-chloro-5-fluorobenzamido)-3-chloro-N-(2,2,2-trifluoroethyl)picolinamide;
4-(3-amino-5-(1-(difluoromethyl)-1H-pyrazol-4-yl)pyridin-4-yl)-2-chloro-N-(5-chloro-6-(2H-1,2,3-triazol-2-yl)pyridin-3-yl)-5-fluorobenzamide;
4-(3-amino-5-(prop-1-en-2-yl)pyridin-4-yl)-2-chloro-N-(5-chloro-6-(2H-1,2,3-triazol-2-yl)pyridin-3-yl)-5-fluorobenzamide;
4-(3-amino-5-ethynylpyridin-4-yl)-2-chloro-N-(5-ethynyl-6-(2H-1,2,3-triazol-2-yl)pyridin-3-yl)-5-fluorobenzamide;
4-(3-amino-5-cyclopropylpyridin-4-yl)-2-chloro-N-(5-chloro-6-(2H-1,2,3-triazol-2-yl)pyridin-3-yl)-5-fluorobenzamide;
4-(3-amino-5-vinylpyridin-4-yl)-2-chloro-N-(5-chloro-6-(2H-1,2,3-triazol-2-yl)pyridin-3-yl)-5-fluorobenzamide;
4-(3-amino-5-(prop-1-yn-1-yl)pyridin-4-yl)-2-chloro-N-(5-chloro-6-(2H-1,2,3-triazol-2-yl)pyridin-3-yl)-5-fluorobenzamide formate;
4-(3-amino-5-(cyclohex-1-en-1-yl)pyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-bromo-2-ethynylpyridin-4-yl)-2-chloro-5-fluorobenzamide;
N-(6-(2H-1,2,3-triazol-2-yl)-5-(trifluoromethyl)pyridin-3-yl)-4-(3-amino-2,5-diethynylpyridin-4-yl)-2-chloro-5-fluorobenzamide;
4-(3-amino-5-(cyclopent-1-en-1-yl)pyridin-4-yl)-2-chloro-N-(5-chloro-6-(2H-1,2,3-triazol-2-yl)pyridin-3-yl)-5-fluorobenzamide;
4-(3-amino-5-ethynylpyridin-4-yl)-2-chloro-5-fluoro-N-(6-(trifluoromethyl)pyridin-3-yl)benzamide;
4-(3-amino-5-(cyclopropylethynyl)pyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-ethynylpyridin-4-yl)-3-fluorobenzamide;
5-(4-(3-amino-5-cyclopropylpyridin-4-yl)-2-chloro-5-fluorobenzamido)-3-chloro-N-(2,2,2-trifluoroethyl)picolinamide;
N-(6-(2H-1,2,3-triazol-2-yl)-5-(trifluoromethyl)pyridin-3-yl)-4-(3-amino-5-bromopyridin-4-yl)-2-chloro-5-fluorobenzamide;
4-(3-amino-5-ethynylpyridin-4-yl)-2-chloro-5-fluoro-N-(2-(trifluoromethyl)pyridin-4-yl)benzamide;
5-(4-(3-amino-5-cyclopropylpyridin-4-yl)-2-chloro-5-fluorobenzamido)-3-cyclopropyl-N-(2,2,2-trifluoroethyl)picolinamide;
N-(6-(2H-1,2,3-triazol-2-yl)-5-(trifluoromethyl)pyridin-3-yl)-4-(5-amino-3-ethynyl-2-fluoropyridin-4-yl)-2-chloro-5-fluorobenzamide;
N-(6-(2H-1,2,3-triazol-2-yl)-5-(trifluoromethyl)pyridin-3-yl)-4-(5-amino-3-bromo-2-fluoropyridin-4-yl)-2-chloro-5-fluorobenzamide;
N-(6-(2H-1,2,3-triazol-2-yl)-5-(trifluoromethyl)pyridin-3-yl)-4-(3-amino-5-(3,6-dihydro-2H-pyran-4-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(3,6-dihydro-2H-pyran-4-yl)pyridin-4-yl)-3-fluorobenzamide;
4-(3-amino-5-cyclopropylpyridin-4-yl)-2-chloro-5-fluoro-N-(2-(trifluoromethyl)pyridin-4-yl)benzamide;
2′-amino-5-chloro-N-(5-cyclopropyl-6-(2H-1,2,3-triazol-2-yl)pyridin-3-yl)-2,4′-difluoro-[1,1′-biphenyl]-4-carboxamide;
2-chloro-4-(3-cyclopropyl-5-(methylamino)pyridin-4-yl)-5-fluoro-N-(2-(trifluoromethyl)pyridin-4-yl)benzamide;
N-(6-(2H-1,2,3-triazol-2-yl)-5-(trifluoromethyl)pyridin-3-yl)-2′-amino-5-chloro-6′-cyclopropyl-2,4′-difluoro-[1,1′-biphenyl]-4-carboxamide;
N-(6-(2H-1,2,3-triazol-2-yl)-5-(trifluoromethyl)pyridin-3-yl)-4-(3-amino-5-(2,5-dihydrofuran-3-yl)pyridin-4-yl)-2-chloro-5-fluorobenzamide;
4-(3-acetyl-5-aminopyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-(4-hydroxycyclohex-1-en-1-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-phenylpyridin-4-yl)-2-chloro-5-fluorobenzamide;
2′-amino-5-chloro-6′-(2-ethoxyethyl)-2,4′-difluoro-N-(2-(trifluoromethyl)pyridin-4-yl)-[1,1′-biphenyl]-4-carboxamide;
2′-amino-5-chloro-2,4′-difluoro-6′-(tetrahydrofuran-2-yl)-N-(2-(trifluoromethyl)pyridin-4-yl)-[1,1′-biphenyl]-4-carboxamide;
2′-amino-5-chloro-2,4′-difluoro-6′-(4-morpholinocyclohexyl)-N-(2-(trifluoromethyl)pyridin-4-yl)-[1,1′-biphenyl]-4-carboxamide;
2′-amino-5-chloro-2,4′-difluoro-6′-(4-hydroxycyclohexyl)-N-(2-(trifluoromethyl)pyridin-4-yl)-[1,1′-biphenyl]-4-carboxamide;
2′-amino-5-chloro-2,4′-difluoro-6′-(2-oxo-1,2-dihydropyridin-4-yl)-N-(2-(trifluoromethyl)pyridin-4-yl)-[1,1′-biphenyl]-4-carboxamide;
2′-amino-5-chloro-2,4′-difluoro-6′-(6-oxo-1,6-dihydropyridin-3-yl)-N-(2-(trifluoromethyl)pyridin-4-yl)-[1,1′-biphenyl]-4-carboxamide;
2′-amino-5-chloro-2,4′-difluoro-6′-(1-methylpiperidin-3-yl)-N-(2-(trifluoromethyl)pyridin-4-yl)-[1,1′-biphenyl]-4-carboxamide;
2′-(1-acetylpiperidin-4-yl)-6′-amino-5-chloro-2,4′-difluoro-N-(2-(trifluoromethyl)pyridin-4-yl)-[1,1′-biphenyl]-4-carboxamide;
2′-amino-5-chloro-2,4′-difluoro-6′-(spiro[2.5]octan-6-yl)-N-(2-(trifluoromethyl)pyridin-4-yl)-[1,1′-biphenyl]-4-carboxamide;
2′-amino-5-chloro-2,4′-difluoro-6′-(2-oxo-1,2-dihydropyrimidin-5-yl)-N-(2-(trifluoromethyl)pyridin-4-yl)-[1,1′-biphenyl]-4-carboxamide;
2′-amino-5-chloro-2,4′-difluoro-6′-(1-(2,2,2-trifluoroacetyl)piperidin-4-yl)-N-(2-(trifluoromethyl)pyridin-4-yl)-[1,1′-biphenyl]-4-carboxamide;
2′-amino-5-chloro-2,4′-difluoro-6′-(4-(trifluoromethyl)cyclohexyl)-N-(2-(trifluoromethyl)pyridin-4-yl)-[1,1′-biphenyl]-4-carboxamide;
2′-amino-5-chloro-2,4′-difluoro-6′-(1-hydroxyethyl)-N-(2-(trifluoromethyl)pyridin-4-yl)-[1,1′-biphenyl]-4-carboxamide;
4-(3-amino-5-bromo-2-chloropyridin-4-yl)-2-chloro-5-fluoro-N-(2-(trifluoromethyl)pyridin-4-yl)benzamide;
4-(3-amino-2,5-dimethylpyridin-4-yl)-2-chloro-5-fluoro-N-(2-(trifluoromethyl)pyridin-4-yl)benzamide;
4-(3-amino-5-bromo-2-methoxypyridin-4-yl)-2-chloro-5-fluoro-N-(2-(trifluoromethyl)pyridin-4-yl)benzamide;
2-chloro-4-(2,3-diamino-5-ethynylpyridin-4-yl)-5-fluoro-N-(2-(trifluoromethyl)pyridin-4-yl)benzamide;
4-(2-amino-5-ethynyl-3-iodopyridin-4-yl)-2-chloro-5-fluoro-N-(2-(trifluoromethyl)pyridin-4-yl)benzamide;
4-(3-amino-5-ethynyl-2-methylpyridin-4-yl)-2-chloro-5-fluoro-N-(2-(trifluoromethyl)pyridin-4-yl)benzamide;
N-(6-(2H-1,2,3-triazol-2-yl)-5-(trifluoromethyl)pyridin-3-yl)-4-(3-amino-5-ethynyl-2-methylpyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2-fluoropyridin-4-yl)-2-chloro-5-fluorobenzamide;
N-(6-(2H-1,2,3-triazol-2-yl)-5-(trifluoromethyl)pyridin-3-yl)-4-(3-amino-2-chloro-5-ethynylpyridin-4-yl)-2-chloro-5-fluorobenzamide;
N-(6-(2H-1,2,3-triazol-2-yl)-5-(trifluoromethyl)pyridin-3-yl)-4-(3-amino-2-cyano-5-ethynylpyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2-methoxypyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-6-fluoro-2-methylpyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2,6-difluoropyridin-4-yl)-2-chloro-5-fluorobenzamide;
N-(6-(2H-1,2,3-triazol-2-yl)-5-(trifluoromethyl)pyridin-3-yl)-4-(3-amino-5-bromo-2-chloropyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2-hydroxypyridin-4-yl)-2-chloro-5-hydroxybenzamide;
N-(6-(2H-1,2,3-triazol-2-yl)-5-(trifluoromethyl)pyridin-3-yl)-4-(3-amino-5-bromo-2-hydroxypyridin-4-yl)-2-chloro-5-hydroxybenzamide;
4-(4-((6-(2H-1,2,3-triazol-2-yl)-5-(trifluoromethyl)pyridin-3-yl)carbamoyl)-5-chloro-2-fluorophenyl)-3-amino-5-ethynylpicolinamide;
N-(6-(2H-1,2,3-triazol-2-yl)-5-(trifluoromethyl)pyridin-3-yl)-4-(3-amino-5-ethynylpyridazin-4-yl)-2-chloro-5-fluorobenzamide;
N-(6-(2H-1,2,3-triazol-2-yl)-5-(trifluoromethyl)pyridin-3-yl)-4-(2-amino-4-ethynyl-6-fluoropyridin-3-yl)-2-chloro-5-fluorobenzamide;
N-(6-(2H-1,2,3-triazol-2-yl)-5-(trifluoromethyl)pyridin-3-yl)-4-(2-amino-4-ethynylpyridin-3-yl)-2-chloro-5-fluorobenzamide;
N-((1S)-1-(4-(4-(3-amino-5-ethynylpyridin-4-yl)-2-chloro-5-fluorobenzamide)phenyl)-2,2,2-trifluoroethyl)-N-methyltetrahydro2H-thiopyran-4-carboxamide1,1-dioxide;
4-(3-amino-2-chloro-5-ethynylpyridin-4-yl)-2-chloro-N-(5-chloro-6-(2H-1,2,3-triazol-2-yl)pyridin-3-yl)-5-fluorobenzamide;
4-(3-amino-2-chloro-5-ethynylpyridin-4-yl)-2-chloro-5-fluoro-N-(2-(trifluoromethyl)pyridin-4-yl)benzamido;
4-(3-amino-5-ethynylpyridin-4-yl)-2-chloro-5-fluoro-N-(5-hydroxy-6-(2H-1,2,3-triazol-2-yl)pyridin-3-yl)benzamido;
4-(3-amino-5-ethynylpyridin-4-yl)-2-chloro-5-fluoro-N-(5-methoxy-6-(2H-1,2,3-triazol-2-yl)pyridin-3-yl)benzamido;
4-(3-amino-5-ethynylpyridin-4-yl)-2-chloro-N-(5-(difluoromethoxy)-6-(2H-1,2,3-triazol-2-yl)pyridin-3-yl)-5-fluorobenzamide; or
N-(6-(2H-1,2,3-triazol-2-yl)-5-(trifluoromethyl)pyridin-3-yl)-4-(5-amino-3-ethynyl-2-fluoropyridin-4-yl)-2-chloro-5-fluorobenzamide.

A second object of the present invention is to provide a pharmaceutical composition comprising a therapeutically effective amount of at least a compound of Formula I, or a stereoisomer, tautomer, deuterated compound, pharmaceutically acceptable salt, prodrug, chelate, non-covalent complex, or solvate thereof as an active ingredient, and at least one pharmaceutically acceptable ingredient, such as carrier or excipient.

A third object of the present invention is to provide use of a compound of Formula I or a stereoisomer, tautomer, deuterated compound, pharmaceutically acceptable salt, prodrug, chelate, non-covalent complex, or solvate thereof for the manufacture of a medicament for the treatment of diseases, syndromes, disorders or obstacles.

In some embodiments, the diseases, syndromes, disorders and obstacles are affected by MALT1 inhibition.

In some embodiments, the diseases, syndromes, disorders and obstacles are selected from diffuse large B-cell lymphoma (DLBCL), mantle cell lymphoma (MCL), follicular lymphoma (FL), mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma and chronic obstructive pulmonary disease (COPD).

A forth object of the present invention is to provide a method of treating diseases, syndromes, disorders or obstacles, wherein the diseases, syndromes, disorders and obstacles are affected by MALT1 inhibition, comprising administering to a patient in need the compound of Formula I, or a stereoisomer, tautomer, deuterated compound, pharmaceutically acceptable salt, prodrug, chelate, non-covalent complex, or solvate thereof.

In some embodiments, the diseases, syndromes, disorders and obstacles are selected from diffuse large B-cell lymphoma (DLBCL), mantle cell lymphoma (MCL), follicular lymphoma (FL), mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma and chronic obstructive pulmonary disease (COPD).

### DEFINITION AND DESCRIPTION

The general chemical terms used in the formula above have their usual meanings. For example, the term “halogen”, as used herein, unless otherwise indicated, means fluoro, chloro, bromo or iodo.

As used herein, unless otherwise indicated, alkyl includes saturated monovalent hydrocarbon radicals having straight, or branched moieties. For example, alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl, and 2-methylpentyl. The alkyl group is preferably a C₁₋₈ alkyl, the C₁₋₈ alkyl is further preferably a C₁₋₆ alkyl, the C₁₋₆ alkyl is further preferably a C₁₋₃ alkyl. Among them, C₁₋₈, as in C₁₋₈ alkyl is defined to identify the group as having 1, 2, 3, 4, 5, 6, 7 or 8 carbon atoms in a linear or branched arrangement.

Alkoxy radicals are oxygen ethers formed from the previously described straight, branched chain or cyclic alkyl groups.

Alkenyl and alkynyl groups include straight, branched chain or cyclic alkenes and alkynes. Likewise, “C₂₋₈ alkenyl” and “C₂₋₈ alkynyl” means an alkenyl or alkynyl radicals having 2, 3, 4, 5, 6, 7 or 8 carbon atoms in a linear or branched arrangement. For example, alkenyl radicals include ethenyl, propenyl, etc. For example, alkynyl radicals include ethynyl, propynyl, etc.

Unless otherwise stated, the term “heteroaryl” as used herein refers to a monocyclic or polycyclic (e.g., fused bicyclic) aromatic heterocycle having at least one heteroatom ring member selected from the group consisting of N, O, and/or S. and wherein the nitrogen or sulfur heteroatom may be optionally oxidized, and the nitrogen heteroatom may be optionally quaternized. The heteroaryl group is preferably a 5-10-membered heteroaryl group, and the 5-10-membered heteroaryl group is further preferably a 5-6-membered heteroaryl group. Examples of heteroaryl groups include, but are not limited to, thienyl, furyl, imidazolyl, isoxazolyl, oxazolyl, pyrazolyl, pyrrolyl, thiazolyl, thiadiazolyl, triazolyl, pyridyl, pyridyl Azinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladenyl, quinolyl or isoquinolyl.

Unless otherwise stated, the term “carbocyclyl” refers to a stable monocyclic, bicyclic or tricyclic ring with carbon atoms; they can be cyclic saturated alkyl groups or structures with partially unsaturated bonds. The carbocyclic group is preferably a C₃₋₁₄ carbocyclyl, the C₃₋₁₄ carbocyclic group is further preferably a C₃₋₈ carbocyclyl, the C₃₋₈ carbocyclic group is further preferably a C₃₋₆ carbocyclic, the C₃₋₆ carbocyclic group is further preferably a C₅₋₆ carbocyclyl. Examples of carbocyclic groups include, but are not limited to, cyclopropyl, cyclobutyl, cyclobutyl, cyclopentyl, and cyclohexenyl.

The term “heterocyclyl”, as used herein, unless otherwise indicated, represents stable heteroatom-containing monocyclic, bicyclic or tricyclic rings, which can be saturated or partially unsaturated. They contain carbon atoms and 1-4 heteroatoms selected from N, O or S, and wherein nitrogen or Sulfur heteroatoms can optionally be oxidized, and nitrogen heteroatoms can optionally be quaternized. Heterocyclyl groups can be attached to any heteroatom or carbon atom, resulting in a stable structure. The heterocyclyl group is preferably a 3-14-membered heterocyclyl group, the 3-14-membered heterocyclyl group is further preferably a 3-8-membered heterocyclyl group or a 5-10-membered heterocyclyl group, and the 3-8-membered heterocyclic group The ring group is further preferably a 3-6-membered heterocyclyl group, and the 3-6-membered heterocyclyl group is further preferably a 5-6-membered heterocyclyl group. Examples of heterocyclic groups include, but are not limited to, oxiranyl, piperazinyl, morpholinyl, piperidinyl, tetrahydropyrrolyl, tetrahydrofuranyl, 4,5,6,7-tetrahydro-2H-[1,2,3]triazolyl[4,5-c]pyridyl, 4,5,6,7-tetrahydro-2H-indazolyl, 2,4,5,6-tetrahydrocyclopentyl[c] ]pyrazolyl, 2,4,5,6-tetrahydrocyclopentyl[d][1,2,3]triazolyl, 5,6,7,8-tetrahydro-[1,2,4]triazole[1,5-a]pyrazinyl, 5,6,7,8-tetrahydro[1,2,4]triazole[4,3-a]pyrazinyl, 4,5,6,7-tetrahydro-1H-phenyl[d]imidazolyl, 2-azaspiro[3.3]heptyl, 2-oxo-7-azaspiro[4.4]nonanyl, 7-aza Bicyclo[2.2.1]heptyl, octahydrocyclopenta[c]pyrrolyl, 3-azabicyclo[3.1.0]hexyl, 3,4-dihydro-2H-benzo[b] [1,4]oxazine, 2H-benzo[b][1,4]oxazine-3(4H)-one, etc.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₂₋₁₂ cycloalkyl, —OR₁₁, SR₁₁, ═O, ═S, —C(O)R₁₁, —C(S)R₁₁, ═NR₁₁, —C(O)OR₁₁, —C(S)OR₁₁, —NR₁₁R₁₂, —C(O)NR₁₁R₁₂, cyano, nitro, —S(O)₂R₁₁, —OS(O₂)OR₁₁, —OS(O)₂R₁₁, —OP(O)(OR₁₁)(OR₁₂); wherein Ru and R₁₂ is independently selected from —H, C₁₋₈ alkyl, C₁₋₈ haloalkyl. In some embodiments, the substituent(s) is independently selected from the group consisting of —F, —Cl, —Br, —I, —OH, trifluromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methyl thio, sulfonyl and acetyl.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, tetrafluoromethoxy, 2-diethylaminoethoxy, 2-ethoxycarbonylethoxy, 3-hydroxypropoxy.

The term “one or more” or “at least one” means one, two, three, four, five, six, seven, eight, nine or more.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The term “stereoisomer” refers to a compound that contains one or more asymmetric centers (or axes) in its structure and is capable of stereoisomerism, including geometric isomers, optical isomers (including atropisomers)) and conformational isomers. The present invention includes all possible diastereoisomers and their racemic mixtures, their substantially pure resolved enantiomers, all possible geometric isomers and their pharmaceutical acceptable salt.

The above Formula I are shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I exists, the present invention includes any possible tautomers and pharmaceutically acceptable salts thereof, and mixtures thereof, except where specifically stated otherwise.

When the compound of Formula I and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a stereoisomer, tautomer, deuterated compound, pharmaceutically acceptable salt, prodrug, chelate, non-covalent complex, or solvate thereof. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example

It is to be understood that both the foregoing general description and the following detailed description are exemplary and explanatory only and are not limiting of any subject matter claimed. All parts and percentages are by weight unless expressly stated otherwise. All temperatures are in degrees Celsius. The raw materials for the synthesis method are not given in this invention, and the reagents can be obtained from commercial sources or synthesized by conventional methods as shown below using commercially available raw materials and reagents.

The following abbreviations have been used in the examples:
POCl₃: Phosphorus oxychloride;
DCM: Dichloromethane;
Dioxane: 1,4-dioxane;
Pd(dppf)Cl₂:1,1′-1,1′-bis(diphenylphosphine)ferrocenepalladium dichloride;
K₂CO₃: Potassium carbonate;
Na₂CO₃: Sodium carbonate;
PE: Petroleum ether;
EA: Ethyl acetate;
h or hrs: hour or hours;
Pd/C: Palladium carbon;
MeOH: Methanol;
Pd(PPh₃)₄: Tetrakis triphenylphosphine palladium;
NaOH: Sodium hydroxide;
LCMS: Liquid chromatography-mass spectrometry;
NaNO₂: Sodium nitrite;
KI: Potassium iodide;
Na₂S₂O₃: Sodium thiosulfate;
H₂O: water;
PTLC: Preparative thin layer chromatography;
TLC: Thin layer chromatography.

### Example 5: Synthesis of Compound A05

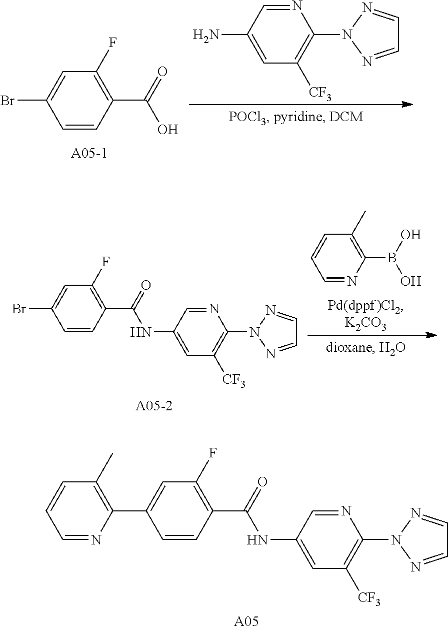

### Step 1: Synthesis of Compound A05-2

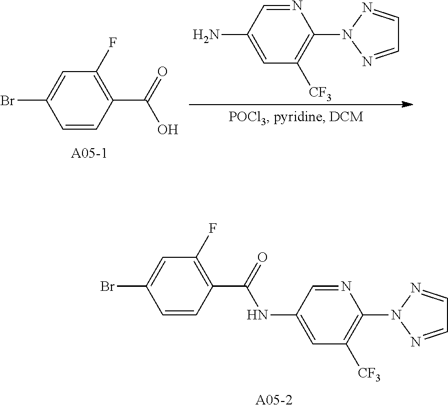 [SMILES: O=C(O)c1ccc(Br)cc1F ; Nc1cnc(-n2nccn2)c(C(F)(F)F)c1 ; O=C(Nc1cnc(-n2nccn2)c(C(F)(F)F)c1)c1ccc(Br)cc1F]

To a solution of A05-1 (4-bromo-2-fluorobenzoic acid) (2.19 g, 10.0 mmol) and 6-(2H-1,2,3-triazol-2-yl)-5-(trifluoromethyl)pyridin-3-amine (2.52 g, 11.0 mmol) in dichloromethane (25 mL) was added pyridine (1.19 g, 15.0 mmol) at room temperature. The mixture was cooled to 0° C. in an ice bath, and then phosphorus oxychloride was added dropwise (4.59 g, 30.0 mmol). The reaction was stirred for 1 hour and quenched by water, extracted with dichloromethane (250 mL). The organic layer was washed with brine, dried over anhydrous Na₂SO₄ and concentrated in vacuum. The residue was purified by flash column chromatography over silica gel to afford compound A05-2 (N-(6-(2H-1,2,3-triazol-2-yl)-5-(trifluoromethyl)pyridin-3-ly)-4-bromo-2-fluorobenzamide) (2.5 g). LC-MS (ES⁺): m/z 430 (M+H)⁺

### Step 2: Synthesis of Compound A05

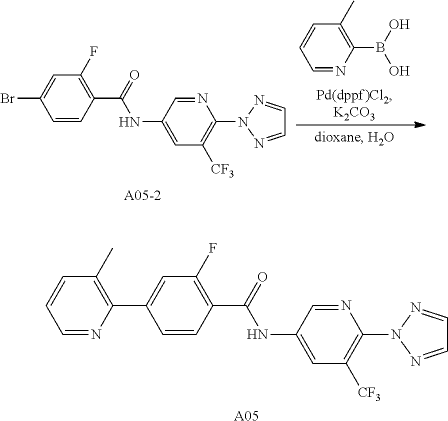

Into a 100-mL round-bottom flask, was placed A05-2 (215 mg, 0.5 mmol), (3-methylpyridin-2-yl)boronic acid (82 mg, 0.6 mmol), Pd(dppf)Cl₂ (36.6 mg, 0.05 mmol), K₂CO₃ (414 mg, 3.0 mmol), dioxane (3 mL) and H₂O (0.5 mL). The resulting mixture was stirred at 100° C. for 2 hours under nitrogen. Removing of the solvent and purifying by column chromatography (PE/EtOAc=1/1) to give compound A05 (47 mg). LC-MS (ES⁺): m/z 443 [M+H]⁺

### Example 12: Synthesis of Compound A12

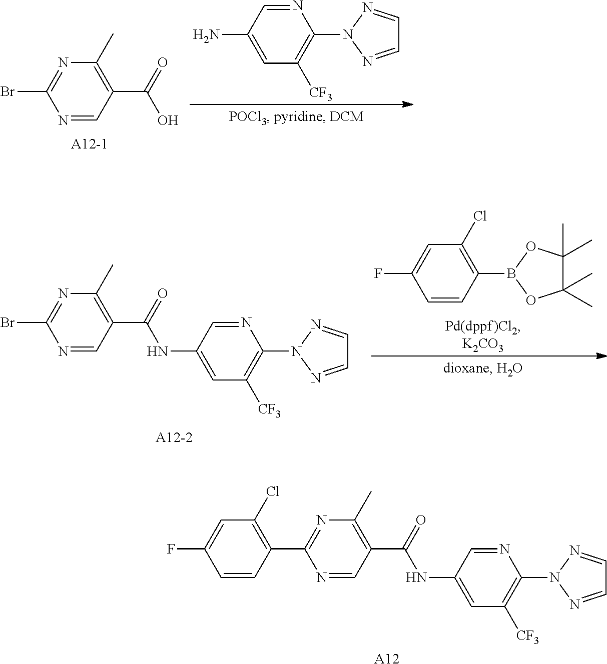 [SMILES: Cc1nc(Br)ncc1C(=O)O ; Nc1cnc(-n2nccn2)c(C(F)(F)F)c1 ; Cc1nc(Br)ncc1C(=O)Nc1cnc(-n2nccn2)c(C(F)(F)F)c1 ; CC1(C)OB(c2ccc(F)cc2Cl)OC1(C)C ; Cc1nc(-c2ccc(F)cc2Cl)ncc1C(=O)Nc1cnc(-n2nccn2)c(C(F)(F)F)c1 ; "A12"]

### Step 1: Synthesis of Compound A12-2

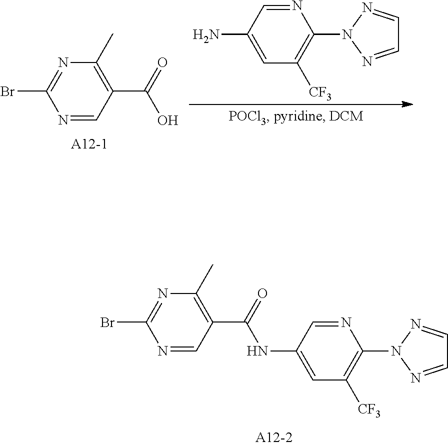 [SMILES: Cc1nc(Br)ncc1C(=O)O ; Nc1cnc(-n2nccn2)c(C(F)(F)F)c1 ; Cc1nc(Br)ncc1C(=O)Nc1cnc(-n2nccn2)c(C(F)(F)F)c1]

To a solution of A12-1 (2.17 g, 10.0 mmol) and 6-(2H-1,2,3-triazol-2-yl)-5-(trifluoromethyl)pyridin-3-amine (2.52 g, 11.0 mmol) in dichloromethane (25 mL), was added pyridine (1.19 g, 15.0 mmol) at room temperature. The mixture was cooled to 0° C. in an ice bath, and then phosphorus oxychloride (4.59 g, 30.0 mmol) was added dropwise. The reaction was stirred at room temperature for 1 hour and quenched by water, extracted with dichloromethane (250 mL). The organic layer was washed with brine, dried over anhydrous Na₂SO₄ and concentrated in vacuum. The residue was purified by flash column chromatography over silica gel to afford compound A12-2 (2.1 g). LC-MS (ES⁺): m/z 428,430 [M+H]⁺

### Step 2: Synthesis of Compound A12

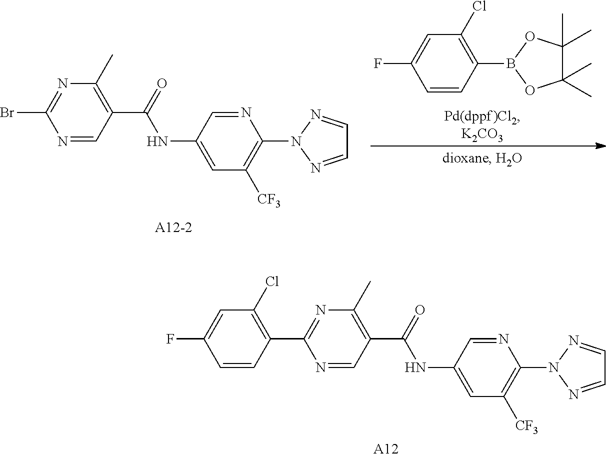 [SMILES: Cc1nc(Br)ncc1C(=O)Nc1cnc(-n2nccn2)c(C(F)(F)F)c1 ; CC1(C)OB(c2ccc(F)cc2Cl)OC1(C)C ; Cc1nc(-c2ccc(F)cc2Cl)ncc1C(=O)Nc1cnc(-n2nccn2)c(C(F)(F)F)c1 ; "A12"]

Into a 50-mL round-bottom flask, was placed A12-2 (215 mg, 0.5 mmol), 2-(2-chloro-4-fluorophenyl)-4,4,5,5-tetramethyl-1,3,2-dioxaborane (154 mg, 0.6 mmol), Pd(dppf)Cl₂, (36.6 mg, 0.05 mmol), K₂CO₃ (414 mg, 3.0 mmol), dioxane (3 mL) and H₂O (0.5 mL). The resulting mixture was stirred at 100° C. for 2 hours under nitrogen. Removing of the solvent and purifying by column chromatography (PE/EtOAc=1/1) to give compound A12 (41 mg), LC-MS (ES⁺): m/z 478 [M+H]⁺

### Example 35: Synthesis of Compound A35

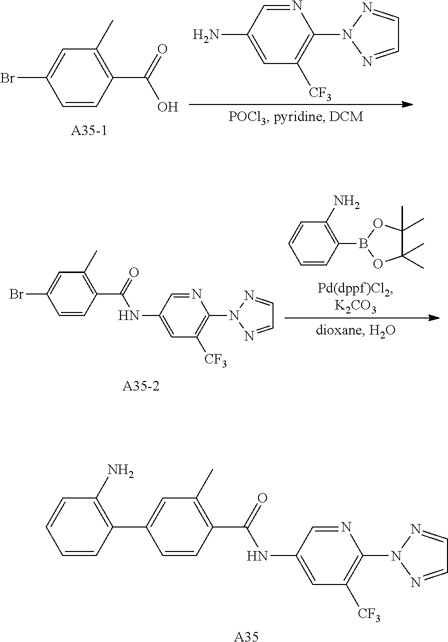 [SMILES: Cc1cc(Br)ccc1C(=O)O ; Nc1cnc(-n2nccn2)c(C(F)(F)F)c1 ; Cc1cc(Br)ccc1C(=O)Nc1cnc(-n2nccn2)c(C(F)(F)F)c1 ; CC1(C)OB(c2ccccc2N)OC1(C)C ; Cc1cc(-c2ccccc2N)ccc1C(=O)Nc1cnc(-n2nccn2)c(C(F)(F)F)c1 ; "A35"]

### Step 1: Synthesis of Compound A35-2

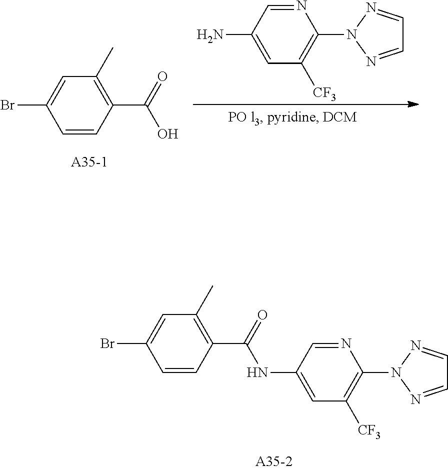 [SMILES: Cc1cc(Br)ccc1C(=O)O ; Nc1cnc(-n2nccn2)c(C(F)(F)F)c1 ; Cc1cc(Br)ccc1C(=O)Nc1cnc(-n2nccn2)c(C(F)(F)F)c1]

To a solution of A35-1 (2.15 g, 10.0 mmol) and 6-(2H-1,2,3-triazol-2-yl)-5-(trifluoromethyl)pyridin-3-amine (2.52 g, 11.0 mmol) in dichloromethane (25 mL), was added pyridine (1.19 g, 15.0 mmol) at room temperature. The mixture was cooled to 0° C. in an ice bath, and then phosphorus oxychloride was added dropwise (4.59 g, 30.0 mmol). The reaction was stirred at room temperature for 1 hour and quenched by water, extracted with dichloromethane (250 mL). The organic layer was washed with brine, dried over anhydrous Na₂SO₄ and concentrated in vacuum. The residue was purified by flash column chromatography over silica gel to afford compound A35-2 (1.8 g), LC-MS (ES⁺): m/z 426 [M+H]⁺

### Step 2: Synthesis of Compound A35

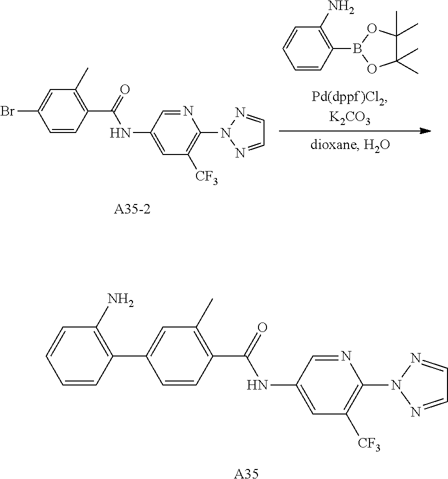 [SMILES: Cc1cc(Br)ccc1C(=O)Nc1cnc(-n2nccn2)c(C(F)(F)F)c1 ; CC1(C)OB(c2ccccc2N)OC1(C)C ; Cc1cc(-c2ccccc2N)ccc1C(=O)Nc1cnc(-n2nccn2)c(C(F)(F)F)c1 ; "A35"]

Into a 50-mL round-bottom flask, was placed A35-2 (213 mg, 0.5 mmol), 2-(4,4,5,5-Tetramethyl-1,3,2-dioxaboran-2-yl) aniline (154 mg, 0.6 mmol), Pd(dppf)Cl₂, (36.6 mg, 0.05 mmol), K₂CO₃ (414 mg, 3.0 mmol), dioxane (3 mL) and H₂O (0.5 mL). The resulting mixture was stirred at 100° C. for 2 hours under nitrogen. Removing of the solvent and purifying by column chromatography to give compound A35 (60 mg), LC-MS (ES⁺): m/z 439 [M+H]⁺

### Example 41: Synthesis of Compound A41

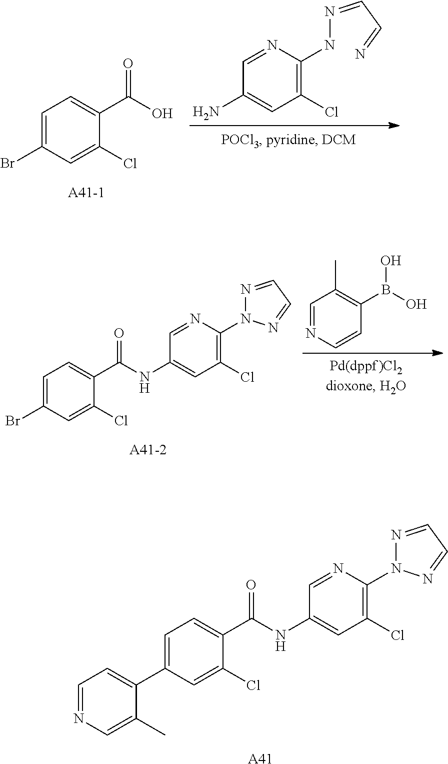

### Step 1: Synthesis of Compound A41-2

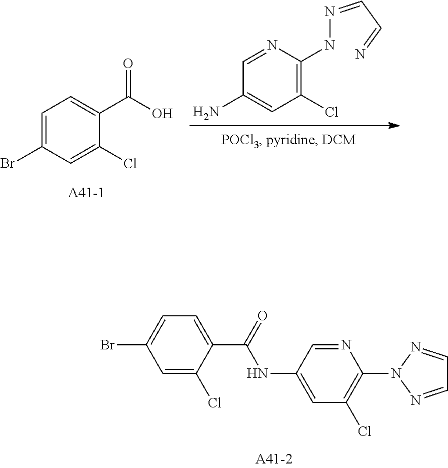 [SMILES: O=C(O)c1ccc(Br)cc1Cl ; N=C/C=N\Nc1ncc(N)cc1Cl ; O=C(Nc1cnc(-n2nccn2)c(Cl)c1)c1ccc(Br)cc1Cl]

To a solution of A41-1 (2.35 g, 10.0 mmol) and 5-chloro-6-(2H-1,2,3-triazol-2-yl)pyridin-3-amine (2.15 g, 11.0 mmol) in dichloromethane (25 mL), was added pyridine (1.19 g, 15.0 mmol) at room temperature. The mixture was cooled to 0° C. in an ice bath, and then phosphorus oxychloride was added dropwise (4.59 g, 30.0 mmol). The reaction was stirred at room temperature for 1 hour and quenched by water, extracted with dichloromethane (250 mL). The organic layer was washed with brine, dried over anhydrous Na₂SO₄ and concentrated in vacuum. The residue was purified by flash column chromatography over silica gel to afford compound A41-2 4-bromo-2-chloro-N-(5-chloro-6-(2H-1,2,3-triazol-2-yl)pyridin-3-yl)benzamide (3.1 g, yellow solid). LC-MS (ES⁺): m/z 412,414 [M+H]⁺

### Step 2: Synthesis of Compound A41

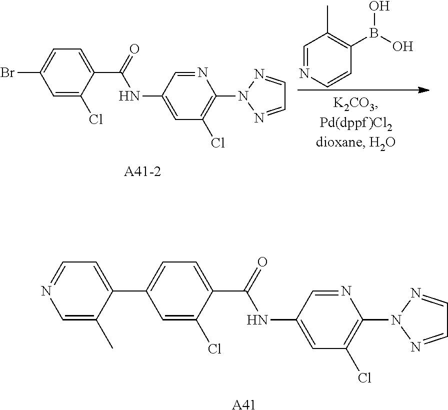

Into a 50-mL round-bottom flask, was placed A41-2 (314 mg, 0.76 mmol), 3-methylpyridine-4-boronic acid (158 mg, 1.15 mmol), Pd(dppf)Cl₂ (58.5 mg, 0.08 mmol), K₂CO₃ (476 mg, 3.45 mmol), dioxane (3 mL) and H₂O (0.5 mL). The resulting mixture was stirred at 75° C. for 2 hours under nitrogen. Removing of the solvent and purifying by column chromatography (PE/EtOAc=1/1) to give compound A41 (50 mg, off-white solid). LC-MS (ES⁺): m/z 425 [M+H]⁺

### Example 42: Synthesis of Compound A42

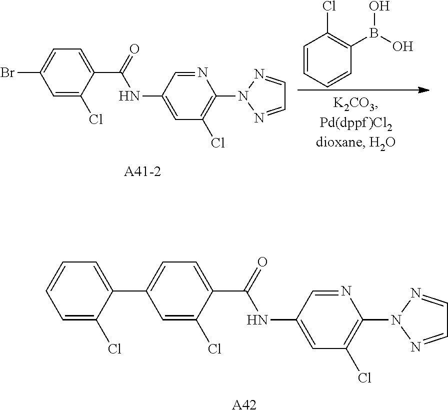

Into a 100-mL round-bottom flask, was placed A41-2 (314 mg, 0.76 mmol), 2-chlorophenylboronic acid (179.8 mg, 1.15 mmol), Pd(dppf)Cl₂ (58.5 mg, 0.08 mmol), K₂CO₃ (476 mg, 3.45 mmol), dioxane (3 mL) and H₂O (0.5 mL). The resulting mixture was stirred at 75° C. for 2 hours under nitrogen. Removing of the solvent and purifying by column chromatography (PE/EtOAc=1/1) to give compound A42 (40 mg off-white solid). LC-MS (ES⁺): m/z 444 [M+H]⁺

### Example 64: Synthesis of Compound A64

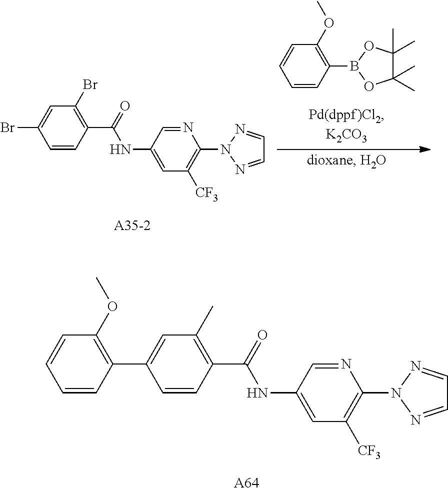 [SMILES: O=C(Nc1cnc(-n2nccn2)c(C(F)(F)F)c1)c1ccc(Br)cc1Br ; COc1ccccc1B1OC(C)(C)C(C)(C)O1 ; COc1ccccc1-c1ccc(C(=O)Nc2cnc(-n3nccn3)c(C(F)(F)F)c2)c(C)c1 ; "A64"]

Into a 100-mL round-bottom flask, was placed A35-2 (213 mg, 0.5 mmol), 2-(2-methoxyphenyl)-4,4,5,5-tetramethyl-1,3,2-dioxaborane (140 mg, 0.6 mmol), Pd(dppf)Cl₂, (36.6 mg, 0.05 mmol), K₂CO₃ (414 mg, 3.0 mmol), dioxane (3 mL) and H₂O (0.5 mL). The resulting mixture was stirred at 100° C. for 2 hours under nitrogen. Removing of the solvent and purifying by column chromatography (PE/EtOAc=1/1) to give compound A64 (50 mg), LC-MS (ES⁺): m/z 454 [M+H]⁺

### Example 98: Synthesis of Compound A98

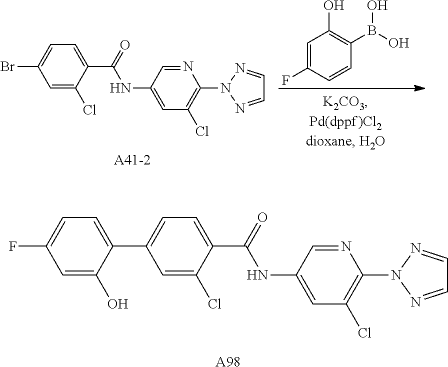

Into a 100-mL round-bottom flask, was placed A41-2 (314 mg, 0.76 mmol), 4-fluoro-2-hydroxyphenylboronic acid (179 mg, 1.15 mmol), Pd(dppf)Cl₂ (58.5 mg, 0.08 mmol), K₂CO₃ (476 mg, 3.45 mmol), dioxane (3 mL) and H₂O (0.5 mL). The resulting mixture was stirred at 75° C. for 2 hours under nitrogen. Removing of the solvent and purifying by column chromatography (PE/EtOAc=1/1) to give compound A98 (49 mg off-white solid). LC-MS (ES⁺): m/z 444 [M+H]⁺

### Example 100: Synthesis of Compound A100

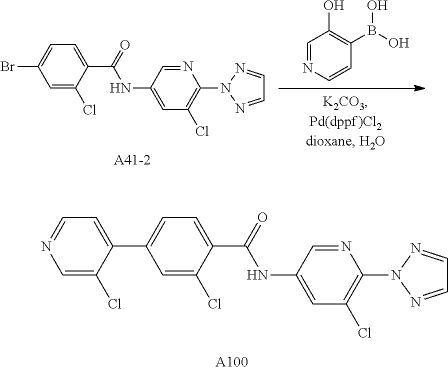 [SMILES: O=C(Nc1cnc(-n2nccn2)c(Cl)c1)c1ccc(Br)cc1Cl ; OB(O)c1ccncc1O ; O=C(Nc1cnc(-n2nccn2)c(Cl)c1)c1ccc(-c2ccncc2Cl)cc1Cl ; "A100"]

Into a 100-mL round-bottom flask, was placed A41-2 (314 mg, 0.76 mmol), 3-chloropyridine-4-boronic acid (180 mg, 1.15 mmol), Pd(dppf)Cl₂ (58.5 mg, 0.08 mmol), K₂CO₃ (476 mg, 3.45 mmol), dioxane (3 mL) and H₂O (0.5 mL). The resulting mixture was stirred at 75° C. for 2 hours under nitrogen. Removing of the solvent and purifying by column chromatography (PE/EtOAc=1/1) to give compound A100 (59 mg off-white solid) LC-MS (ES⁺): m/z 445 [M+H]⁺

### Example 102: Synthesis of Compound A102

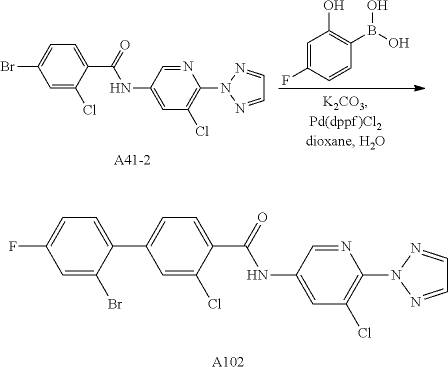

Into a 100-mL round-bottom flask, was placed A41-2 (314 mg, 0.76 mmol), 2-Bromo-4-fluorophenylboronic acid (252 mg, 1.15 mmol), Pd(dppf)Cl₂ (58.5 mg, 0.08 mmol), K₂CO₃ (476 mg, 3.45 mmol), dioxane (3 mL) and H₂O (0.5 mL). The resulting mixture was stirred at 75° C. for 2 hours. Removing of the solvent and purifying by column chromatography (PE/EtOAc=1/1) to give compound A102 (53 mg off-white solid). LC-MS (ES⁺): m/z 506,508 [M+H]⁺

### A102:

¹H NMR (500 MHz, DMSO-d₆) δ 11.41 (s, 1H), 8.76 (d, J=87.2 Hz, 2H), 8.19 (s, 2H), 7.60 (dd, J=126.7, 61.0 Hz, 6H).

### Example 104: Synthesis of Compound A104

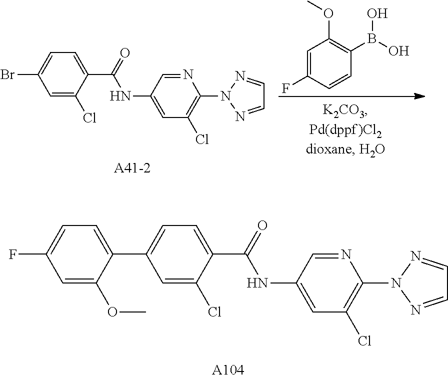

Into a 100-mL round-bottom flask, was placed A41-2 (314 mg, 0.76 mmol), 4-fluoro-2-methoxyphenylboronic acid (196 mg, 1.15 mmol), Pd(dppf)Cl₂ (58.5 mg, 0.08 mmol), K₂CO₃ (476 mg, 3.45 mmol), dioxane (3 mL) and H₂O (0.5 mL). The resulting mixture was stirred at 75° C. for 2 hours under nitrogen. Removing of the solvent and purifying by column chromatography (PE/EtOAc=1/1) to give compound A104 (57 mg off-white solid). LC-MS (ES⁺): m/z 458 [M+H]⁺

### Example 105: Synthesis of Compound A105

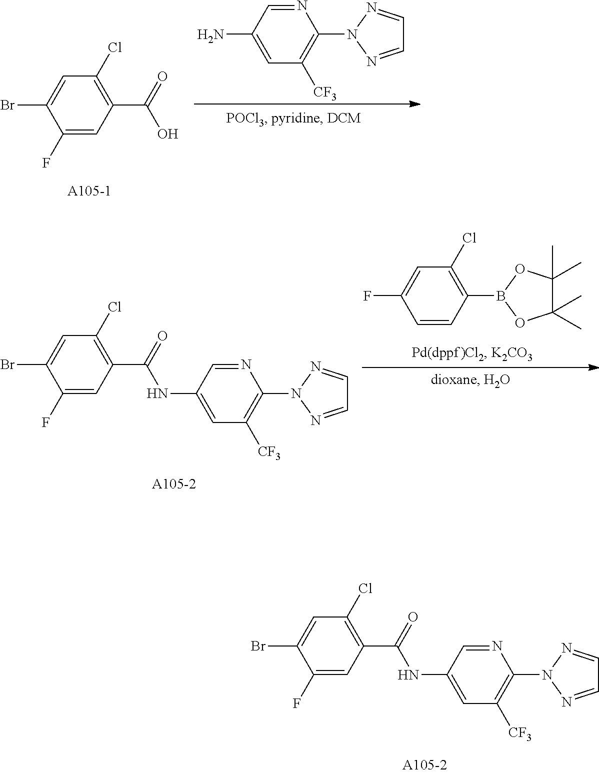 [SMILES: O=C(O)c1cc(F)c(Br)cc1Cl ; Nc1cnc(-n2nccn2)c(C(F)(F)F)c1 ; O=C(Nc1cnc(-n2nccn2)c(C(F)(F)F)c1)c1cc(F)c(Br)cc1Cl ; CC1(C)OB(c2ccc(F)cc2Cl)OC1(C)C ; O=C(Nc1cnc(-n2nccn2)c(C(F)(F)F)c1)c1cc(F)c(Br)cc1Cl]

### Step 1: Synthesis of Compound A105-2

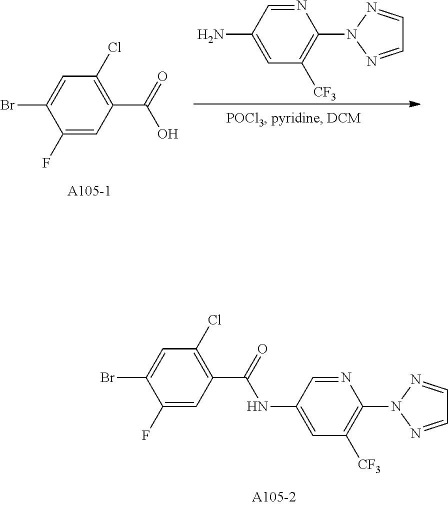 [SMILES: O=C(O)c1cc(F)c(Br)cc1Cl ; Nc1cnc(-n2nccn2)c(C(F)(F)F)c1 ; O=C(Nc1cnc(-n2nccn2)c(C(F)(F)F)c1)c1cc(F)c(Br)cc1Cl]

To a solution of A105-1 (2.54 g, 10.0 mmol) and 6-(2H-1,2,3-triazol-2-yl)-5-(trifluoromethyl)pyridin-3-amine (2.52 g, 11.0 mmol) in dichloromethane (25 mL), was added pyridine (1.19 g, 15.0 mmol) at room temperature. The mixture was cooled to 0° C. in an ice bath, and then phosphorus oxychloride was added dropwise (4.59 g, 30.0 mmol). The reaction was stirred at room temperature for 1 hour, and quenched by water, extracted with dichloromethane (250 mL). The organic layer was washed with brine, dried over anhydrous Na₂SO₄ and concentrated in vacuum. The residue was purified by flash column chromatography over silica gel to afford compound A105-2 (2.5 g). LC-MS (ES⁺): m/z 464 [M+H]⁺

### Step 2: Synthesis of Compound A105

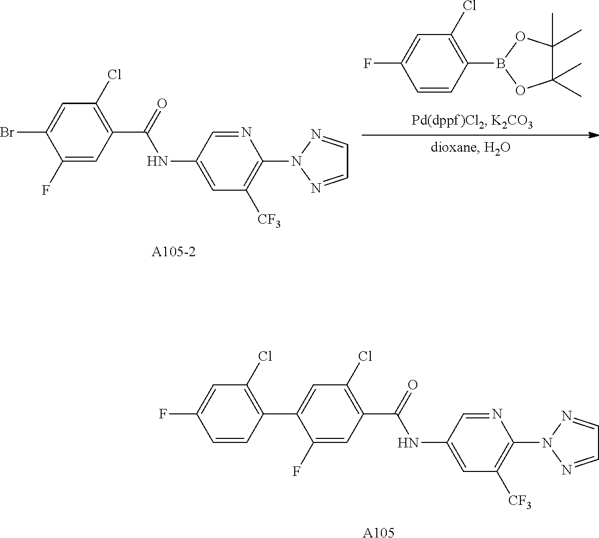 [SMILES: O=C(Nc1cnc(-n2nccn2)c(C(F)(F)F)c1)c1cc(F)c(Br)cc1Cl ; CC1(C)OB(c2ccc(F)cc2Cl)OC1(C)C ; O=C(Nc1cnc(-n2nccn2)c(C(F)(F)F)c1)c1cc(F)c(-c2ccc(F)cc2Cl)cc1Cl ; "A105"]

Into a 100-mL round-bottom flask, was placed A105-2 (232 mg, 0.5 mmol), 2-(2-Chloro-4-fluorophenyl)-4,4,5,5-tetramethyl-1,3,2-dioxaborane (154 mg, 0.6 mmol), Pd(dppf)Cl₂ (40 mg, 0.05 mmol), K₂CO₃ (414 mg, 3.0 mmol), dioxane (3 mL) and H₂O (0.5 mL). The resulting mixture was stirred at 100° C. for 2 hours under nitrogen. Removing of the solvent and purifying by column chromatography (PE/EtOAc=1/1) to give compound A105 (40 mg), LC-MS (ES⁺): m/z 514 [M+H]+

### A105:

¹H NMR (500 MHz, DMSO-d₆) δ 11.60 (s, 1H), 9.12 (d, J=2.4 Hz, 1H), 8.89 (d, J=2.4 Hz, 1H), 8.21 (s, 2H), 7.86 (d, J=9.1 Hz, 1H), 7.74 (d, J=6.3 Hz, 1H), 7.69 (dd, J=8.9, 2.7 Hz, 1H), 7.60 (dd, J=8.6, 6.1 Hz, 1H), 7.41 (td, J=8.4, 2.6 Hz, 1H).

### Example 67: Synthesis of Compound A67

### Step 1: Synthesis of Compound A67

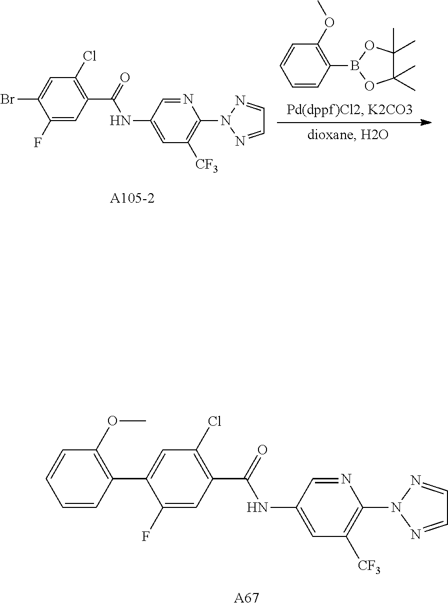 [SMILES: O=C(Nc1cnc(-n2nccn2)c(C(F)(F)F)c1)c1cc(F)c(Br)cc1Cl ; COc1ccccc1B1OC(C)(C)C(C)(C)O1 ; COc1ccccc1-c1cc(Cl)c(C(=O)Nc2cnc(-n3nccn3)c(C(F)(F)F)c2)cc1F ; "A67"]

Into a 100-mL round-bottom flask, was placed A105-2 (115 mg, 0.25 mmol), 2-methoxyphenylboronic acid (45.6 mg, 0.3 mmol), Pd(dppf)Cl₂, (18.3 mg, 0.025 mmol), K₂CO₃ (207 mg, 1.5 mmol), dioxane (3 mL) and H₂O (0.5 mL). The resulting mixture was stirred at 100° C. for 2 hours under nitrogen. Removing of the solvent and purifying by column chromatography (PE/EtOAc=1/1) to give compound A67 (18 mg). LC-MS (ES⁺): m/z 492 [M+H]⁺

### Example 69: Synthesis of Compound A69

### Step 1: Synthesis of Compound A69

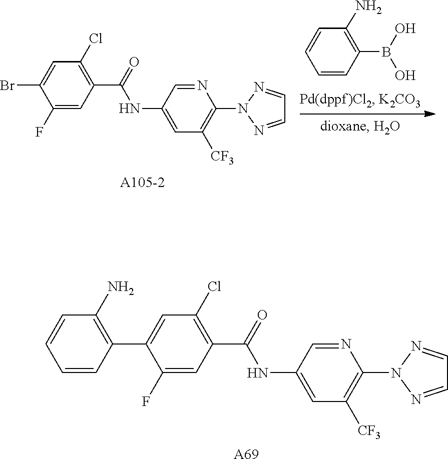 [SMILES: O=C(Nc1cnc(-n2nccn2)c(C(F)(F)F)c1)c1cc(F)c(Br)cc1Cl ; Nc1ccccc1B(O)O ; Nc1ccccc1-c1cc(Cl)c(C(=O)Nc2cnc(-n3nccn3)c(C(F)(F)F)c2)cc1F ; "A69"]

Into a 100-mL round-bottom flask, was placed A105-2 (116 mg, 0.25 mmol), 2-aminophenylboronic acid (41 mg, 0.3 mmol), Pd(dppf)Cl₂, (18.3 mg, 0.025 mmol), K₂CO₃ (207 mg, 1.5 mmol), dioxane (3 mL) and H₂O (0.5 mL). The resulting mixture was stirred at 100° C. for 2 hours under nitrogen. Removing of the solvent and purifying by column chromatography (PE/EtOAc=1/1) to give compound A69 (24 mg), LC-MS (ES⁺): m/z 477 [M+H]⁺

### Example 110: Synthesis of Compound A110

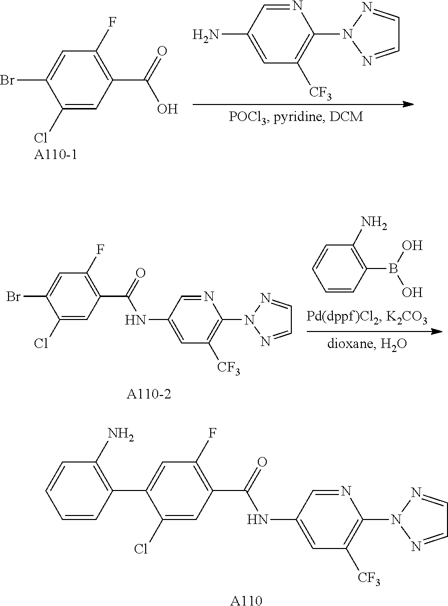

### Step 1: Synthesis of Compound A110-2

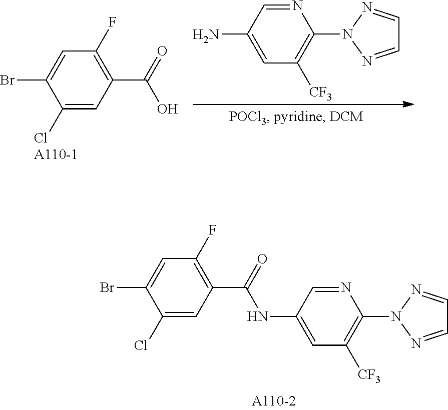 [SMILES: O=C(O)c1cc(Cl)c(Br)cc1F ; Nc1cnc(-n2nccn2)c(C(F)(F)F)c1 ; O=C(Nc1cnc(-n2nccn2)c(C(F)(F)F)c1)c1cc(Cl)c(Br)cc1F]

To a solution of A110-1 (2.53 g, 10.0 mmol) and 6-(2H-1,2,3-triazol-2-yl)-5-(trifluoromethyl)pyridin-3-amine (2.52 g, 11.0 mmol) in dichloromethane (25 mL) was added pyridine (1.19 g, 15.0 mmol) at room temperature. The mixture was cooled to 0° C. in an ice bath, and then phosphorus oxychloride was added dropwise (4.59 g, 30.0 mmol). The reaction was stirred at room temperature for 1 hour, and quenched by water, extracted with dichloromethane (250 mL). The organic layer was washed with brine, dried over anhydrous Na₂SO₄ and concentrated in vacuum. The residue was purified by flash column chromatography over silica gel to afford compound A110-2 (1.9 g). LC-MS (ES⁺): m/z 464,466 [M+H]⁺

### Step 2: Synthesis of Compound A110

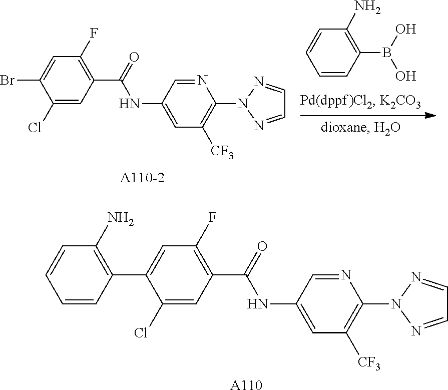

Into a 100-mL round-bottom flask, was placed A110-2 (215 mg, 0.5 mmol), 2-aminophenylboronic acid (82.2 mg, 0.6 mmol), Pd(dppf)Cl₂ (36.6 mg, 0.05 mmol), K₂CO₃ (414 mg, 3.0 mmol), dioxane (3 mL) and H₂O (0.5 mL). The resulting mixture was stirred at 100° C. for 2 hours under nitrogen. Removing of the solvent and purifying by column chromatography (PE/EtOAc=1/1) to give compound A110 (55 mg). LC-MS (ES⁺): m/z 477[M+H]⁺

### Example 126: Synthesis of Compound A126

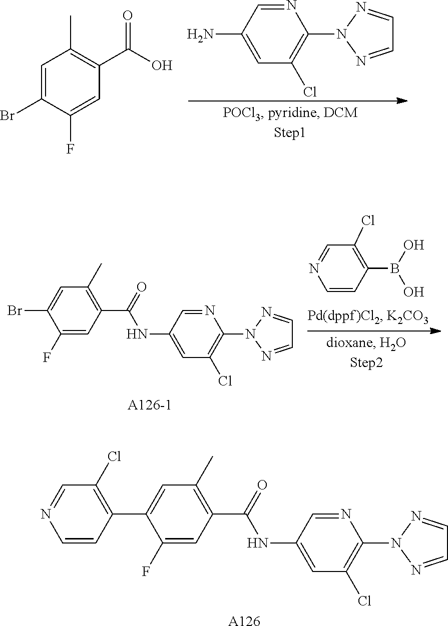

### Step 1: Synthesis of Compound A126-1

To a solution of 5-chloro-6-(2H-1,2,3-triazol-2-yl)pyridin-3-amine (1.95 g) and 4-bromo-5-fluoro-2-methylbenzoic acid (2.33 g) in 20 mL of DCM, was added pyridine (1.6 g) at room temperature. The mixture was cooled to 0° C. in an ice bath, and then POCl₃ (3.1 g) was added dropwise. The reaction was stirred at room temperature for 2 hours and quenched by water, extracted with dichloromethane (100 mL). The organic layer was washed with brine, dried over anhydrous Na₂SO₄ and concentrated in vacuum. The residue was purified by flash column chromatography over silica gel to afford compound A126-1 (3.7 g, white solid). LC-MS (ES⁺): m/z 410,412[M+H]⁺

### Step 2: Synthesis of Compound A126

Into a 100-mL round-bottom flask, was placed A126-1 (41 mg), (3-Chloropyridin-4-yl)boronic acid (16 mg), dioxane (1 mL), water (0.2 mL), Pd(dppf)Cl₂ (5 mg) and K₂CO₃ (30 mg). The resulting mixture was stirred at 90° C. and stirred for 16 hours under nitrogen. Removing of the solvent and purifying by column chromatography (DCM/MeOH=40/1) to give compound A126 (21 mg, white solid). LC-MS (ES⁺): m/z 443[M+H]⁺

### Example 113: Synthesis of Compound A113

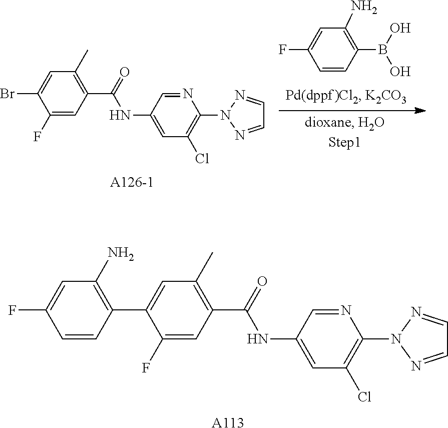 [SMILES: Cc1cc(Br)c(F)cc1C(=O)Nc1cnc(-n2nccn2)c(Cl)c1 ; Nc1cc(F)ccc1B(O)O ; Cc1cc(-c2ccc(F)cc2N)c(F)cc1C(=O)Nc1cnc(-n2nccn2)c(Cl)c1 ; "A113"]

Into a 100-mL round-bottom flask, was placed A126-1 (41 mg), (2-Amino-4-fluorophenyl)boronic acid (16 mg), dioxane (1 mL), water (0.2 mL), Pd(dppf)Cl₂ (5 mg) and K₂CO₃ (30 mg). The resulting mixture was stirred at 90° C. for 16 hours under nitrogen. Removing of the solvent and purifying by column chromatography (DCM/MeOH=40/1) to give compound A113 (29 mg, white solid). LC-MS (ES⁺): m/z 441[M+H]⁺

A113: ¹H NMR (500 MHz, DMSO-d₆) δ 11.19 (d, J=22.7 Hz, 1H), 8.86 (dd, J=19.1, 2.3 Hz, 1H), 8.68 (dd, J=18.5, 2.2 Hz, 1H), 8.18 (d, J=1.9 Hz, 2H), 7.83-7.25 (m, 2H), 7.17-6.45 (m, 2H), 2.43 (d, J=18.8 Hz, 3H).

### Example 114: Synthesis of Compound A114

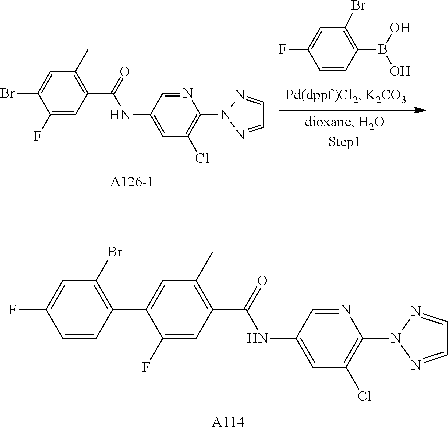 [SMILES: Cc1cc(Br)c(F)cc1C(=O)Nc1cnc(-n2nccn2)c(Cl)c1 ; OB(O)c1ccc(F)cc1Br ; Cc1cc(-c2ccc(F)cc2Br)c(F)cc1C(=O)Nc1cnc(-n2nccn2)c(Cl)c1 ; "A114"]

Into a 100-mL round-bottom flask, was placed A126-1 (41 mg), (2-bromo-4-fluorophenyl)boronic acid (26 mg), dioxane (1 mL), water (0.2 mL), Pd(dppf)Cl₂ (5 mg) and K₂CO₃ (30 mg). The resulting mixture was stirred at 90° C. for 16 hours under nitrogen. Removing of the solvent and purifying by column chromatography (DCM/MeOH=40/1) to give compound A114 (19 mg, white solid). LC-MS (ES⁺): m/z 504,506[M+H]⁺ A114:

¹H NMR (500 MHz, DMSO-d₆) δ 11.20 (s, 1H), 8.86 (d, J=2.3 Hz, 1H), 8.69 (d, J=2.2 Hz, 1H), 8.19 (s, 2H), 7.79 (dd, J=8.6, 2.6 Hz, 1H), 7.62 (d, J=9.6 Hz, 1H), 7.50 (dd, J=8.6, 6.1 Hz, 1H), 7.42 (td, J=8.4, 2.6 Hz, 1H), 7.37 (d, J=7.1 Hz, 1H), 2.45 (s, 3H).

### Example 30: Synthesis of Compound A130

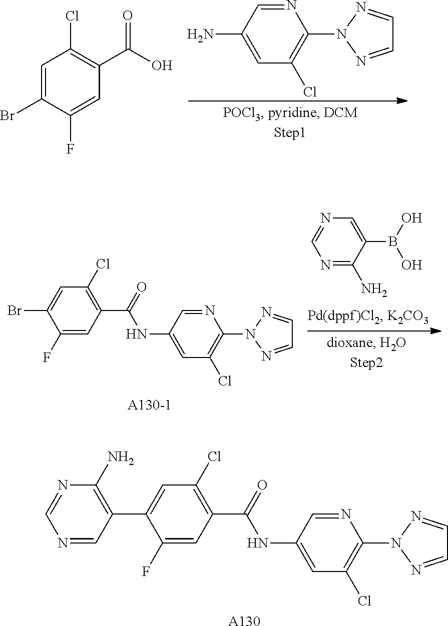 [SMILES: O=C(O)c1cc(F)c(Br)cc1Cl ; Nc1cnc(-n2nccn2)c(Cl)c1 ; O=C(Nc1cnc(-n2nccn2)c(Cl)c1)c1cc(F)c(Br)cc1Cl ; Nc1ncncc1B(O)O ; Nc1ncncc1-c1cc(Cl)c(C(=O)Nc2cnc(-n3nccn3)c(Cl)c2)cc1F ; "A130"]

### Step 1: Synthesis of Compound A130-1

To a solution of 5-chloro-6-(2H-1,2,3-triazol-2-yl)pyridin-3-amine (1.95 g) and 4-bromo-2-chloro-5-fluorobenzoic acid (2.53 g) in dichloromethane (20 mL) was added pyridine (1.6 g) at room temperature. The mixture was cooled to 0° C. in an ice bath, and then phosphorus oxychloride was added dropwise (3.1 g). The reaction was stirred at room temperature for 2 hours and quenched by water, extracted with dichloromethane (50 mL×2). The organic layer was washed with brine, dried over anhydrous Na₂SO₄ and concentrated in vacuum. The residue was purified by flash column chromatography over silica gel (PE/EA=2/1) to afford compound A130-1 4-bromo-2-chloro-N-(5-chloro-6-(2H-1,2,3-triazol-2-yl)pyridin-3-yl)-5-fluorobenzamide (3.9 g). LC-MS (ES⁺): m/z 430 [M+H]⁺

### Step 2: Synthesis of Compound A130

Into a 100-mL round-bottom flask, was placed A130-1 (43 mg), (4-aminopyrimidin-5-yl)boronic acid (14 mg), Pd(dppf)Cl₂ (5 mg), K₂CO₃ (30 mg), dioxane (1 mL) and H₂O (0.2 mL). The resulting mixture was stirred at 90° C. for 16 hours under nitrogen. The reaction solution was diluted with EA (5 mL) washed with saturated NaCl solution, and dried over anhydrous sodium sulfate. The organic phase was concentrated and purified by column chromatography to obtain A130 (23 mg) as a white solid. LC-MS (ES⁺): m/z 445 [M+H]⁺

### Example 133: Synthesis of Compound A133

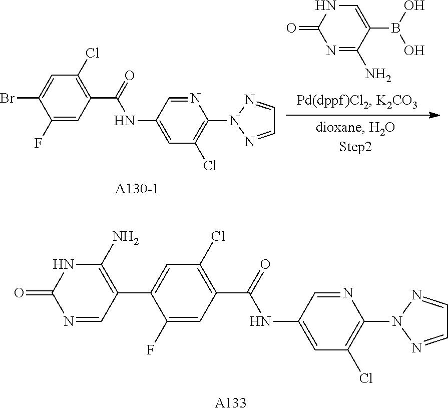 [SMILES: O=C(Nc1cnc(-n2nccn2)c(Cl)c1)c1cc(F)c(Br)cc1Cl ; Nc1nc(=O)[nH]cc1B(O)O ; Nc1[nH]c(=O)ncc1-c1cc(Cl)c(C(=O)Nc2cnc(-n3nccn3)c(Cl)c2)cc1F ; "A133"]

Into a 100-mL round-bottom flask, was placed A130-1 (43 mg), (4-amino-2-oxo-1,2-dihydropyrimidin-5-yl)boronic acid (16 mg), Pd(dppf)Cl₂ (5 mg), K₂CO₃ (30 mg), dioxane (1 mL) and H₂O (0.2 mL). The resulting mixture was stirred at 90° C. for 16 hours under nitrogen. The reaction solution was diluted with EA (5 mL) washed with saturated NaCl solution, and dried over anhydrous sodium sulfate. The organic phase was concentrated and purified by column chromatography to obtain A133 (22 mg) as a white solid. LC-MS (ES⁺): m/z 461 [M+H]+

### Example 147: Synthesis of Compound A147

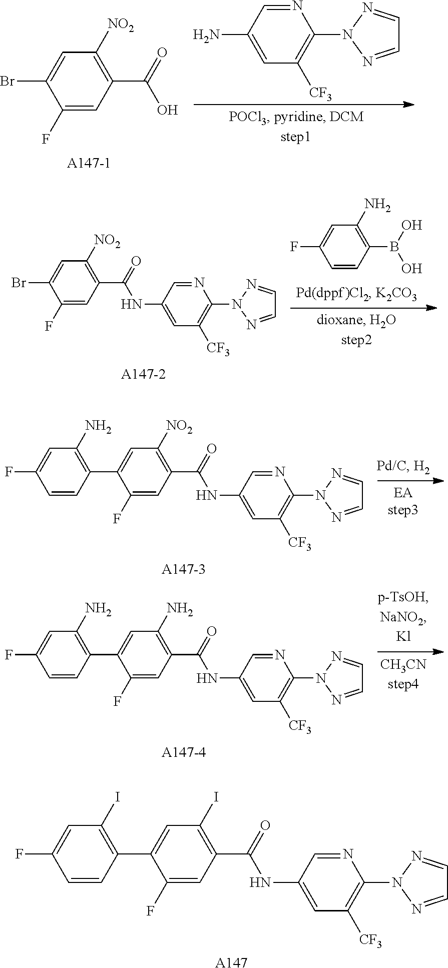

### Step 1: Synthesis of Compound A147-2

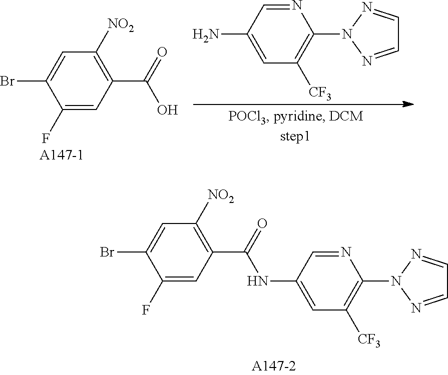 [SMILES: O=C(O)c1cc(F)c(Br)cc1[N+](=O)[O-] ; Nc1cnc(-n2nccn2)c(C(F)(F)F)c1 ; O=C(Nc1cnc(-n2nccn2)c(C(F)(F)F)c1)c1cc(F)c(Br)cc1[N+](=O)[O-]]

To a solution of A147-1 (2.64 g, 10.0 mmol) and 6-(2H-1,2,3-triazol-2-yl)-5-(trifluoromethyl)pyridin-3-amine (2.15 g, 11.0 mmol) in dichloromethane (25 mL) was added pyridine (1.19 g, 15.0 mmol) at room temperature. The mixture was cooled to 0° C. in an ice bath, and then phosphorus oxychloride was added dropwise (4.59 g, 30.0 mmol). The reaction was stirred for 1 hour and quenched by water, extracted with dichloromethane (125 mL×2). The organic layer was washed with brine, dried over anhydrous Na₂SO₄ and concentrated in vacuum. The residue was purified by flash column chromatography over silica gel to afford compound A147-2 (2.6 g). LC-MS (ES⁺): m/z 475 [M+H]⁺

### Step 2: Synthesis of Compound A147-3

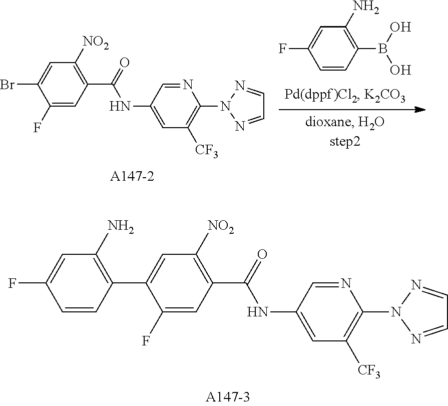 [SMILES: O=C(Nc1cnc(-n2nccn2)c(C(F)(F)F)c1)c1cc(F)c(Br)cc1[N+](=O)[O-] ; Nc1cc(F)ccc1B(O)O ; Nc1cc(F)ccc1-c1cc([N+](=O)[O-])c(C(=O)Nc2cnc(-n3nccn3)c(C(F)(F)F)c2)cc1F]

Into a 100-mL round-bottom flask, was placed A147-2 (950 mg, 2 mmol), (2-amino-4-fluorophenyl)boronic acid (388 mg, 2.5 mmol), Pd(dppf)Cl₂ (18.3 mg, 0.025 mmol), K₂CO₃ (207 mg, 1.5 mmol), dioxane (3 mL) and H₂O (0.5 mL). The resulting mixture was stirred at 100° C. for 2 hours under nitrogen. Removing of the solvent and purifying by column chromatography to give compound A147-3 (700 mg). LC-MS (ES⁺): m/z 506 [M+H]⁺

### Step 3: Synthesis of Compound A147-4

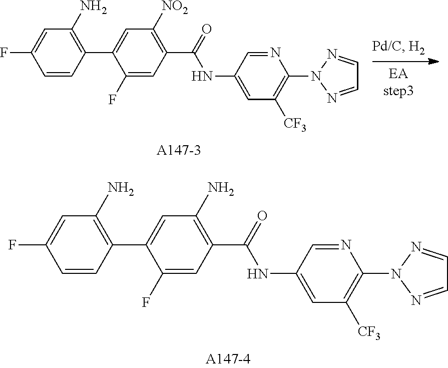 [SMILES: Nc1cc(F)ccc1-c1cc([N+](=O)[O-])c(C(=O)Nc2cnc(-n3nccn3)c(C(F)(F)F)c2)cc1F ; Nc1cc(-c2ccc(F)cc2N)c(F)cc1C(=O)Nc1cnc(-n2nccn2)c(C(F)(F)F)c1]

To a solution of A147-3 (700 mg, 2 mmol) in ethyl acetate (20 mL) was added Pd/C (10%, 100 mg) under nitrogen atmosphere in a 100 mL round bottom flask. The flask was then vacuumed and flushed with hydrogen. The reaction mixture was hydrogenated at room temperature for 2 hours under hydrogen atmosphere using a hydrogen balloon, then filtered through a Celite pad and concentrated under reduced pressure. The residue was used without further purification and compound A147-4 (500 mg) was obtained. LC-MS (ES⁺): m/z 476 [M+H]⁺

### Step 4: Synthesis of Compound A147

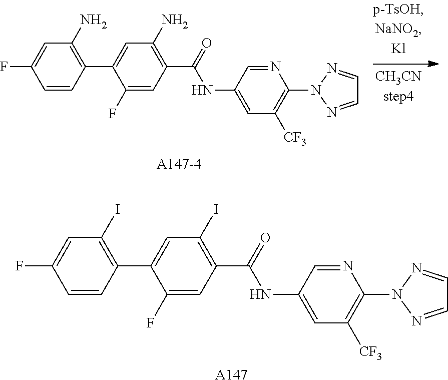 [SMILES: Nc1cc(-c2ccc(F)cc2N)c(F)cc1C(=O)Nc1cnc(-n2nccn2)c(C(F)(F)F)c1 ; O=C(Nc1cnc(-n2nccn2)c(C(F)(F)F)c1)c1cc(F)c(-c2ccc(F)cc2I)cc1I ; "A147"]

To a solution of A147-4 (95 mg, 0.2 mmol) in acetonitrile (5 mL) was added p-toluenesulfonic acid (172 mg, 1 mmol) at 0° C. An aqueous solution of sodium nitrite (69 mg, 1 mmol, dissolved in 1 mL of water) was added to the resulting mixture and stirred for 0.5 hour and then potassium iodide (166 mg, 1 mmol, dissolved in 1 mL water) was added. The mixture was stirred at 25° C. for 1 hour, then raised to 100° C. and continued for 0.5 hour. The reaction was quenched by aqueous sodium thiosulfate solution and extracted by Eethyl acetate (50 mL×2). The organic layer was concentrated and purified by column chromatography to obtain compound A147 (22 mg). LC-MS (ES⁺): m/z 698 [M+H]⁺

### Example 157: Synthesis of Compound A157

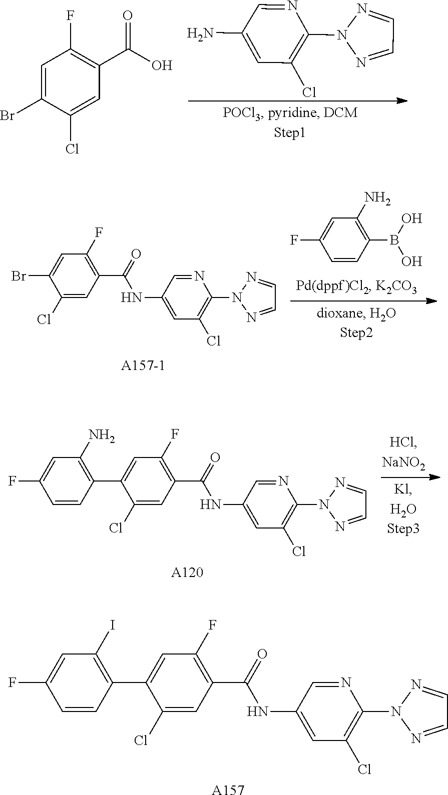

Step 1: Synthesis of Compound A157-1

To a solution of 5-chloro-6-(2H-1,2,3-triazol-2-yl)pyridin-3-amine (1.95 g) and 4-bromo-5-chloro-2-fluorobenzoic acid (2.53 g) in dichloromethane (20 mL) was added pyridine (1.6 g) at room temperature. The mixture was cooled to 0° C. in an ice bath, and then phosphorus oxychloride was added dropwise (3.1 g). The reaction was stirred for 2 hour at room temperature and quenched by water, extracted with dichloromethane (50 mL×2). The organic layer was washed with brine, dried over anhydrous Na₂SO₄ and concentrated in vacuum. The residue was purified by flash column chromatography over silica gel (PE/EA=2/1) to afford compound A157-1 4-Bromo-5-chloro-N-(5-chloro-6-(2H-1,2,3-triazol-2-yl)pyridin-3-yl)-2-fluorobenzamide (4.1 g) as a white solid. LC-MS (ES⁺): m/z 430 [M+H]⁺

### Step 2: Synthesis of Compound A120

Into a 50-mL round-bottom flask, was placed A157-1 (430 mg), (2-amino-4-fluorophenyl)boronic acid (155 mg), Pd(dppf)Cl₂ (50 mg), K₂CO₃ (300 mg), dioxane (10 mL) and H₂O (2 mL). The resulting mixture was stirred at 90° C. for 16 hours under nitrogen. Removing of the solvent and purifying by column chromatography (DCM/MeOH=40/1) to give compound A120 (345 mg) as a white solid. LC-MS (ES⁺): m/z 461 [M+H]⁺

### A120:

¹H NMR (500 MHz, DMSO-d₆) δ 11.23 (s, 1H), 8.86 (d, J=2.3 Hz, 1H), 8.67 (d, J=2.3 Hz, 1H), 8.19 (s, 2H), 7.98 (d, J=6.5 Hz, 1H), 7.42 (d, J=10.2 Hz, 1H), 6.93 (dd, J=8.4, 6.7 Hz, 1H), 6.54 (dd, J=11.8, 2.6 Hz, 1H), 6.42 (td, J=8.5, 2.6 Hz, 1H), 5.20 (s, 2H).

### Step 3: Synthesis of Compound A157

A120 (92 mg) was dissolved in concentrated hydrochloric acid (5 mL). At 0° C., an aqueous solution of sodium nitrite (20 mg, dissolved in 1 mL water) was added to the resulting mixture and stirred for 1 hour and then potassium iodide (34 mg, dissolved in 1 mL water) was added. The mixture was stirred at room temperature for 2 hours. The reaction was quenched by aqueous sodium thiosulfate solution and extracted by ethyl acetate. The organic layer was concentrated and purified by column chromatography (DCM/MeOH=40/1) to obtain compound A157 (65 mg) as a white solid. LC-MS (ES⁺): m/z 572 [M+H]⁺

### Example 124: Synthesis of Compound A124

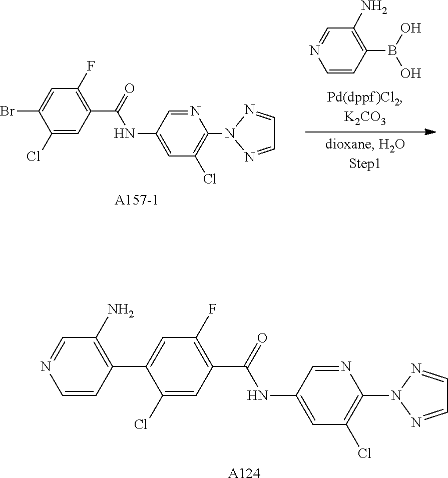 [SMILES: O=C(Nc1cnc(-n2nccn2)c(Cl)c1)c1cc(Cl)c(Br)cc1F ; Nc1cnccc1B(O)O ; Nc1cnccc1-c1cc(F)c(C(=O)Nc2cnc(-n3nccn3)c(Cl)c2)cc1Cl ; "A124"]

Into a 10-mL round-bottom flask, was placed A157-1 (43 mg), (3-aminopyridin-4-yl)boronic acid (15 mg), Pd(dppf)Cl₂ (5 mg), K₂CO₃ (30 mg), dioxane (1 mL) and H₂O (0.2 mL). The resulting mixture was stirred at 90° C. for 16 hours under nitrogen. Removing of the solvent and purifying by column chromatography (DCM/MeOH=40/1) to give compound A124 (26 mg) as a white solid. LC-MS (ES⁺): m/z 444 [M+H]⁺

### Example 220: Synthesis of Compound A220

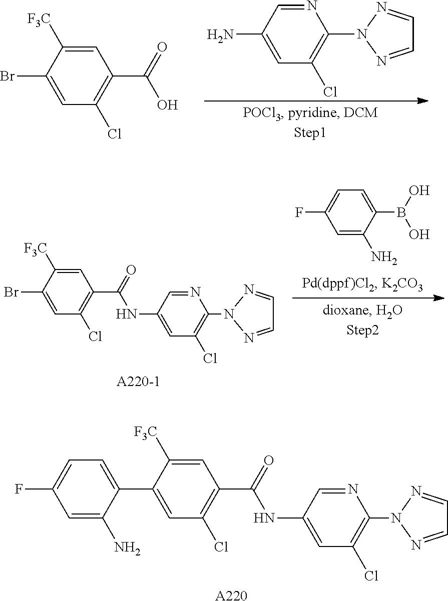 [SMILES: O=C(O)c1cc(C(F)(F)F)c(Br)cc1Cl ; Nc1cnc(-n2nccn2)c(Cl)c1 ; O=C(Nc1cnc(-n2nccn2)c(Cl)c1)c1cc(C(F)(F)F)c(Br)cc1Cl ; Nc1cc(F)ccc1B(O)O ; Nc1cc(F)ccc1-c1cc(Cl)c(C(=O)Nc2cnc(-n3nccn3)c(Cl)c2)cc1C(F)(F)F ; "A220"]

### Step 1: Synthesis of Compound A220-1

To a solution of 5-chloro-6-(2H-1,2,3-triazol-2-yl)pyridin-3-amine (1.95 g) and 4-bromo-2-chloro-5-(fluorotrifluoromethyl)benzoic acid (3.03 g) in dichloromethane (20 mL) was added pyridine (1.6 g) at room temperature. The mixture was cooled to 0° C. in an ice bath, and then phosphorus oxychloride was added dropwise (3.1 g). The reaction was stirred at room temperature for 2 hours and quenched by water, extracted with dichloromethane (50 mL×2). The organic layer was washed with brine, dried over anhydrous Na₂SO₄ and concentrated in vacuum. The residue was purified by flash column chromatography over silica gel to afford compound A220-1 4-bromo-2-chloro-N-(5-chloro-6-(2H-1,2,3-triazol-2-yl)pyridin-3-yl)-5-(fluorotrifluoromethyl)benzamide (4.1 g) as a white solid. LC-MS (ES⁺): m/z 480 [M+H]⁺

### Step 2: Synthesis of Compound A220

Into a 10-mL round-bottom flask, was placed A220-1 (48 mg), (2-amino-4-fluorophenyl)boronic acid (15 mg), Pd(dppf)Cl₂ (5 mg), K₂CO₃ (30 mg), dioxane (1 mL) and H₂O (0.2 mL). The resulting mixture was stirred at 90° C. for 16 hours under nitrogen. Removing of the solvent and purifying by column chromatography (DCM/MeOH=40/1) to give compound A220 (25 mg) as a white solid. LC-MS (ES⁺): m/z 541 [M+H]⁺

### Example 274: Synthesis of Compound A274

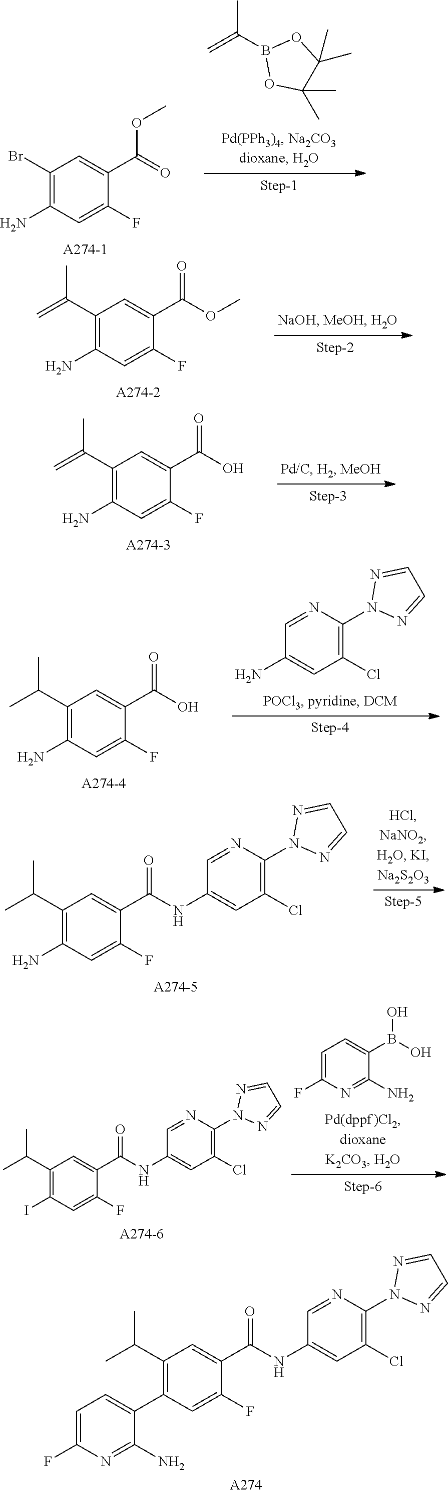

### Step 1: Synthesis of Compound A274-2

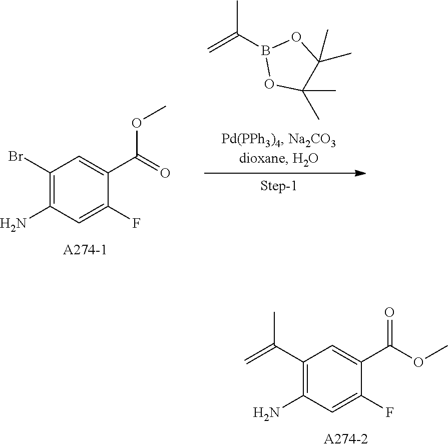

Into a 50-mL round-bottom flask, was placed A274-1 (1.23 g, 5 mmol), 4,4,5,5-tetramethyl-2-(prop-1-en-2-yl)-1,3,2-dioxaborolane (1.68 g, 10 mmol), Pd(PPh₃)₄ (578 mg, 0.5 mmol), Na₂CO₃ (1.59 g, 15 mmol), dioxane (12 mL) and H₂O (3 mL). The resulting mixture was stirred at 85° C. for 3 hours under nitrogen. Removing of the solvent and purifying by column chromatography to give compound A274-2 (800 mg, 3.83 mmol) as a yellow solid.

### Step 2: Synthesis of Compound A274-3

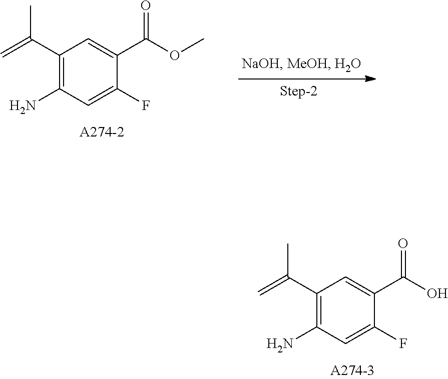

Into a flask, A274-2 (800 mg, 3.83 mmol), NaOH (459 mg, 11.5 mmol), methanol (8 mL) and H₂O (4 mL) were added and reacted at room temperature for 16 hours. The end of the reaction were monitored by LCMS. Adjusted the pH to 3 with concentrated hydrochloric acid under an ice bath, solid precipitated, filtered, and dried to obtain a yellow solid A274-3 (500 mg).

### Step 3: Synthesis of Compound A274-4

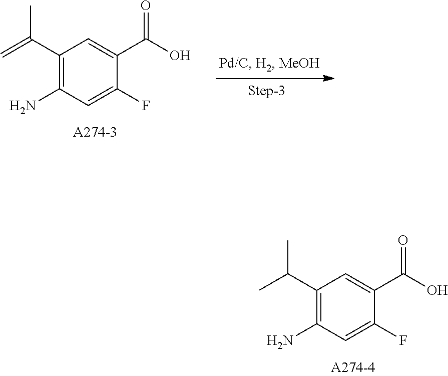

To a solution of A274-3 (500 mg, 2.56 mmol) in methanol (8 mL) was added Pd/C (10%, 250 mg) under nitrogen atmosphere in a round bottom flask. The flask was then vacuumed and flushed with hydrogen. The reaction mixture was hydrogenated at room temperature for 5 hours under hydrogen atmosphere using a hydrogen balloon, then filtered through a Celite pad and concentrated under reduced pressure. The residue was used without further purification and compound A274-4 (450 mg) was obtained.

### Step 4: Synthesis of Compound A274-5

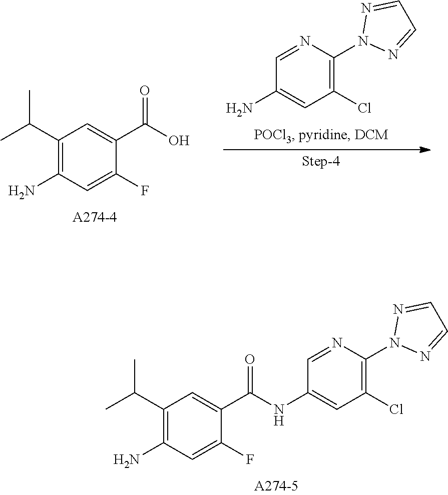 [SMILES: CC(C)c1cc(C(=O)O)c(F)cc1N ; Nc1cnc(-n2nccn2)c(Cl)c1 ; CC(C)c1cc(C(=O)Nc2cnc(-n3nccn3)c(Cl)c2)c(F)cc1N]

To a solution of A274-4 (450 mg, 2.28 mmol) and 5-chloro-6-(2H-1,2,3-triazol-2-yl)pyridin-3-amine (585 mg, 3 mmol) in dichloromethane (5 mL) was added pyridine (540 mg, 6.84 mmol) at room temperature. The mixture was cooled to 0° C. in an ice bath, and then phosphorus oxychloride was added dropwise (459 mg, 3 mmol). The reaction was stirred for 1 hour and quenched by water, extracted with dichloromethane (25 mL×2). The organic layer was washed with brine, dried over anhydrous Na₂SO₄ and concentrated in vacuum. The residue was purified by flash column chromatography over silica gel to afford compound A274-5 (600 mg, 1.60 mmol) as a reddish brown solid.

### Step 5: Synthesis of Compound A274-6

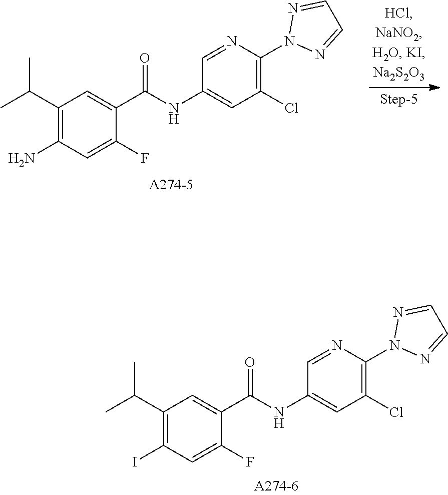 [SMILES: CC(C)c1cc(C(=O)Nc2cnc(-n3nccn3)c(Cl)c2)c(F)cc1N ; CC(C)c1cc(C(=O)Nc2cnc(-n3nccn3)c(Cl)c2)c(F)cc1I]

At 0° C., to a solution of A274-5 (600 mg, 1.60 mmol) in 2N HCl (6 mL, 2 mol·L⁻¹ HCl), an aqueous solution of sodium nitrite (138 mg, 2 mmol, dissolved in 2 mL of water) was added to the resulting mixture and stirred for 15 minutes and then potassium iodide (332 mg, 2 mmol, dissolved in 2 mL water) was added. The mixture was stirred at 0° C. for 15 minutes, 4° C. for 10 minutes, 100° C. for 10 minutes, and then stirred at 0° C. for 5 minutes. The reaction was quenched by aqueous sodium thiosulfate solution and extracted by Eethyl acetate (25 mL×2). The organic layer was concentrated and purified by column chromatography to obtain compound A274-6 (200 mg, 0.41 mmol) as a reddish brown solid.

### Step 6: Synthesis of Compound A274

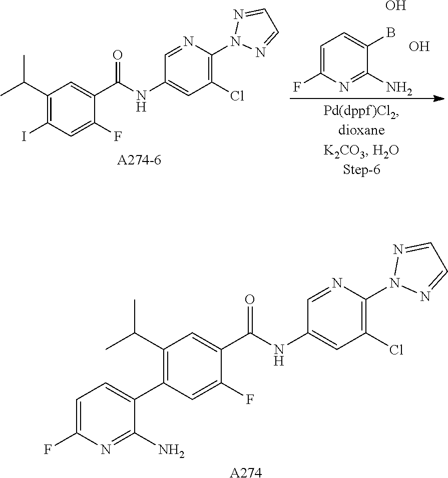

Into a round-bottom flask, was placed A274-6 (200 mg, 0.41 mmol), (2-amino-6-fluoropyridin-3-yl)boronic acid (94 mg, 0.6 mmol), Pd(dppf)Cl₂ (29 mg, 0.04 mmol), K₂CO₃ (170 mg, 1.23 mmol), dioxane (2 mL) and H₂O (0.5 mL). The resulting mixture was stirred at 75° C. for 2 hours under nitrogen. Removing of the solvent and purifying by column chromatography to give compound A274 (20 mg). LC-MS (ES⁺): m/z 470 [M+H]⁺

According to the synthesis method of the above example, select appropriate raw materials and/or marketed example intermediates to synthesize the example compounds in the table below. The raw materials and reagents used are all commercially available.

| | | | MS |
|---|---|---|---|
| Example | Structure | Chemical Name | [M + H]⁺ |
| Example 1 (A01) | 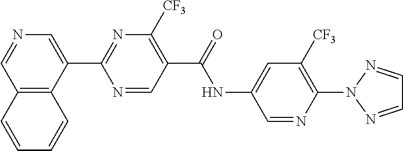 [SMILES: O=C(Nc1cnc(-n2nccn2)c(C(F)(F)F)c1)c1cnc(-c2cncc3ccccc23)nc1C(F)(F)F] | N-(6-(2H-1,2,3-triazol-2- yl)-5- (trifluoromethyl)pyridin-3- yl)-2-(isoquinolin-4-yl)-4- (trifluoromethyl)pyrimidine- 5-carboxamide | 531 |
| Example 2 (A02) | 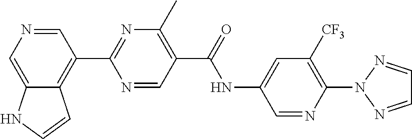 [SMILES: Cc1nc(-c2cncc3[nH]ccc23)ncc1C(=O)Nc1cnc(-n2nccn2)c(C(F)(F)F)c1] | N-(6-(2H-1,2,3-triazol-2- yl)-5- (trifluoromethyl)pyridin-3- yl)-4-methyl-2-(1H- pyrrolo[2,3-c]pyridin-4- yl)pyrimidine-5- carboxamide | 466 |
| Example 3 (A03) | 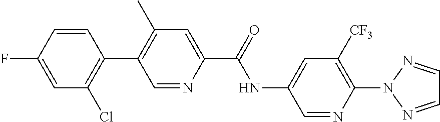 [SMILES: Cc1cc(C(=O)Nc2cnc(-n3nccn3)c(C(F)(F)F)c2)ncc1-c1ccc(F)cc1Cl] | N-(6-(2H-1,2,3-triazol-2- yl)-5- (trifluoromethyl)pyridin-3- yl)-5-(2-chloro-4- fluorophenyl)-4- methylpicolinamide | 477 |
| Example 4 (A04) | 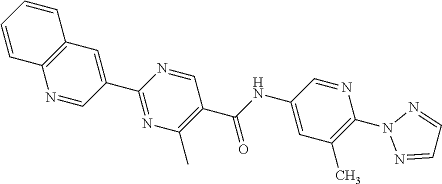 [SMILES: Cc1cc(NC(=O)c2cnc(-c3cnc4ccccc4c3)nc2C)cnc1-n1nccn1] | N-(6-(2H-1,2,3-triazol-2- yl)-5- (trifluoromethyl)pyridin-3- yl)-4-methyl-2-(quinolin-3- yl)pyrimidine-5- carboxamide | 477 |
| Example 6 (A06) | 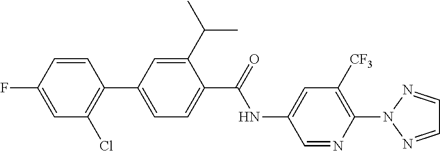 [SMILES: CC(C)c1cc(-c2ccc(F)cc2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-chloro-4′-fluoro-3- isopropyl-[1,1′-biphenyl]-4- carboxamide | 504 |
| Example 7 (A07) | 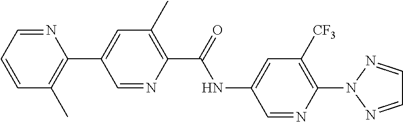 [SMILES: Cc1cc(-c2ncccc2C)cnc1C(=O)Nc1cnc(-n2nccn2)c(C(F)(F)F)c1] | N-(6-(2H-1,2,3-triazol-2- yl)-5- (trifluoromethyl)pyridin-3- yl)-3,5′-dimethyl-[2,3′- bipyridine]-6′-carboxamide | 440 |
| Example 8 (A08) | 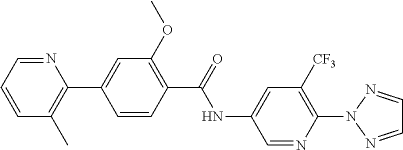 [SMILES: COc1cc(-c2ncccc2C)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2-methoxy-4-(3- methylpyridin-2- yl)benzamide | 455 |
| Example 9 (A09) | 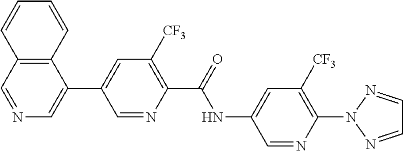 [SMILES: O=C(Nc1cnc(-n2nccn2)c(C(F)(F)F)c1)c1ncc(-c2cncc3ccccc23)cc1C(F)(F)F] | N-(6-(2H-1,2,3-triazol-2- yl)-5- (trifluoromethyl)pyridin-3- yl)-5-(isoquinolin-4-yl)-3- (trifluoromethyl)picolinamide | 530 |
| Example 10 (A10) | 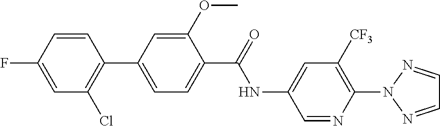 [SMILES: COc1cc(-c2ccc(F)cc2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-chloro-4′-fluoro-3- methoxy-[1,1′-biphenyl]-4- carboxamide | 492 |
| Example 11 (A11) | 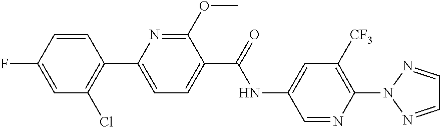 [SMILES: COc1nc(-c2ccc(F)cc2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-6-(2-chloro-4- fluorophenyl)-2- methoxynicotinamide | 493 |
| Example 13 (A13) | 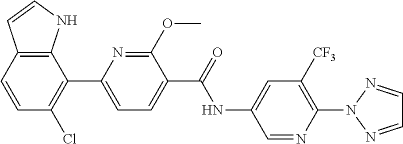 [SMILES: COc1nc(-c2c(Cl)ccc3cc[nH]c23)ccc1C(=O)Nc1cnc(-n2nccn2)c(C(F)(F)F)c1] | N-(6-(2H-1,2,3-triazol-2- yl)-5- (trifluoromethyl)pyridin-3- yl)-6-(6-chloro-1H-indol-7- yl)-2-methoxynicotinamide | 514 |
| Example 14 (A14) | 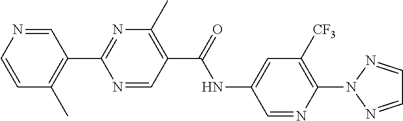 [SMILES: Cc1ccncc1-c1ncc(C(=O)Nc2cnc(-n3nccn3)c(C(F)(F)F)c2)c(C)n1] | N-(6-(2H-1,2,3-triazol-2- yl)-5- (trifluoromethyl)pyridin-3- yl)-4-methyl-2-(4- methylpyridin-3- yl)pyrimidine-5- carboxamide | 441 |
| Example 15 (A15) | 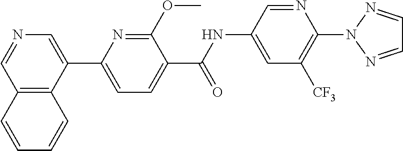 [SMILES: COc1nc(-c2cncc3ccccc23)ccc1C(=O)Nc1cnc(-n2nccn2)c(C(F)(F)F)c1] | N-(6-(2H-1,2,3-triazol-2- yl)-5- (trifluoromethyl)pyridin-3- yl)-6-(isoquinolin-4-yl)-2- methoxynicotinamide | 492 |
| Example 16 (A16) | 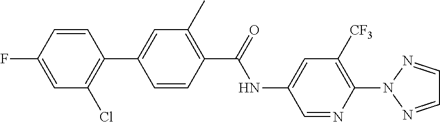 [SMILES: Cc1cc(-c2ccc(F)cc2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-chloro-4′-fluoro-3- methyl-[1,1′-biphenyl]-4- carboxamide | 476 |
| Example 17 (A17) | 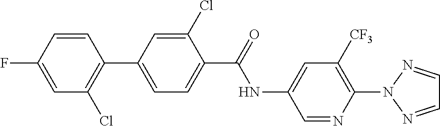 [SMILES: O=C(Nc1cnc(-n2nccn2)c(C(F)(F)F)c1)c1ccc(-c2ccc(F)cc2Cl)cc1Cl] | N-(6-(2H-1,2,3-triazol-2- yl)-5- (trifluoromethyl)pyridin-3- yl)-2′,3-dichloro-4′-fluoro- [1,1′-biphenyl]-4- carboxamide | 496 |
| Example 18 (A18) | 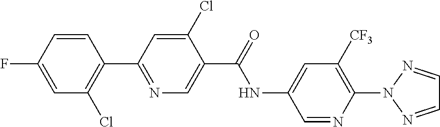 [SMILES: O=C(Nc1cnc(-n2nccn2)c(C(F)(F)F)c1)c1cnc(-c2ccc(F)cc2Cl)cc1Cl] | N-(6-(2H-1,2,3-triazol-2- yl)-5- (trifluoromethyl)pyridin-3- yl)-4-chloro-6-(2-chloro-4- fluorophenyl)nicotinamide | 497 |
| Example 19 (A19) | 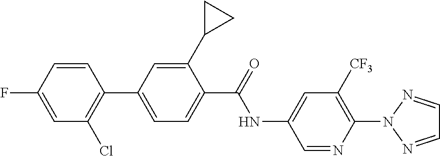 [SMILES: O=C(Nc1cnc(-n2nccn2)c(C(F)(F)F)c1)c1ccc(-c2ccc(F)cc2Cl)cc1C1CC1] | N-(6-(2H-1,2,3-triazol-2- yl)-5- (trifluoromethyl)pyridin-3- yl)-2′-chloro-3- cyclopropyl-4′-fluoro-[1,1′- biphenyl]-4-carboxamide | 502 |
| Example 20 (A20) | 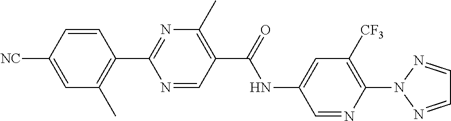 [SMILES: Cc1cc(C#N)ccc1-c1ncc(C(=O)Nc2cnc(-n3nccn3)c(C(F)(F)F)c2)c(C)n1] | N-(6-(2H-1,2,3-triazol-2- yl)-5- (trifluoromethyl)pyridin-3- yl)-2-(4-cyano-2- methylphenyl)-4- methylpyrimidine-5- carboxamide | 465 |
| Example 21 (A21) | 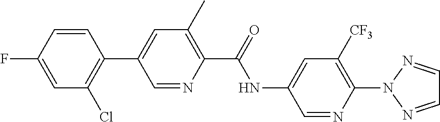 [SMILES: Cc1cc(-c2ccc(F)cc2Cl)cnc1C(=O)Nc1cnc(-n2nccn2)c(C(F)(F)F)c1] | N-(6-(2H-1,2,3-triazol-2- yl)-5- (trifluoromethyl)pyridin-3- yl)-5-(2-chloro-4- fluorophenyl)-3- methylpicolinamide | 477 |
| Example 22 (A22) | 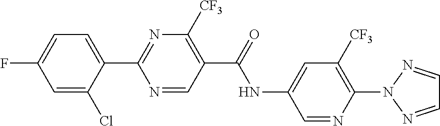 [SMILES: O=C(Nc1cnc(-n2nccn2)c(C(F)(F)F)c1)c1cnc(-c2ccc(F)cc2Cl)nc1C(F)(F)F] | N-(6-(2H-1,2,3-triazol-2- yl)-5- (trifluoromethyl)pyridin-3- yl)-2-(2-chloro-4- fluorophenyl)-4- (trifluoromethyl)pyrimidine- 5-carboxamide | 532 |
| Example 23 (A23) | 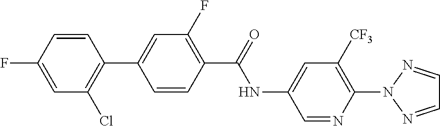 [SMILES: O=C(Nc1cnc(-n2nccn2)c(C(F)(F)F)c1)c1ccc(-c2ccc(F)cc2Cl)cc1F] | N-(6-(2H-1,2,3-triazol-2- yl)-5- (trifluoromethyl)pyridin-3- yl)-2′-chloro-3,4′-difluoro- [1,1′-biphenyl]-4- carboxamide | 480 |
| Example 24 (A24) | 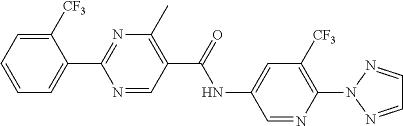 [SMILES: Cc1nc(-c2ccccc2C(F)(F)F)ncc1C(=O)Nc1cnc(-n2nccn2)c(C(F)(F)F)c1] | N-(6-(2H-1,2,3-triazol-2- yl)-5- (trifluoromethyl)pyridin-3- yl)-4-methyl-2-(2- (trifluoromethyl)phenyl) pyrimidine-5-carboxamide | 494 |
| Example 25 (A25) | 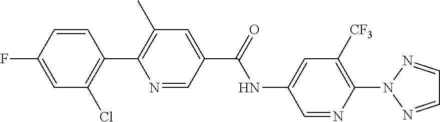 [SMILES: Cc1cc(C(=O)Nc2cnc(-n3nccn3)c(C(F)(F)F)c2)cnc1-c1ccc(F)cc1Cl] | N-(6-(2H-1,2,3-triazol-2- yl)-5- (trifluoromethyl)pyridin-3- yl)-6-(2-chloro-4- fluorophenyl)-5- methylnicotinamide | 477 |
| Example 26 (A26) | 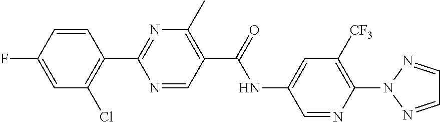 [SMILES: Cc1nc(-c2ccc(F)cc2Cl)ncc1C(=O)Nc1cnc(-n2nccn2)c(C(F)(F)F)c1] | 2-(2-chloro-4- fluorophenyl)-N-(5-chloro- 6-(2H-1,2,3-triazol-2- yl)pyridin-3-yl)-4- methylpyrimidine-5- carboxamide | 444 |
| Example 27 (A27) | 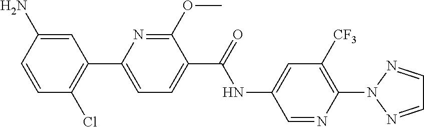 [SMILES: COc1nc(-c2cc(N)ccc2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-6-(5-amino-2- chlorophenyl)-2- methoxynicotinamide | 490 |
| Example 28 (A28) | 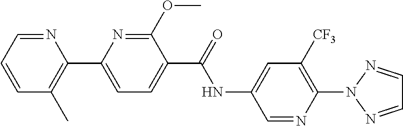 [SMILES: COc1nc(-c2ncccc2C)ccc1C(=O)Nc1cnc(-n2nccn2)c(C(F)(F)F)c1] | N-(6-(2H-1,2,3-triazol-2- yl)-5- (trifluoromethyl)pyridin-3- yl)-6-methoxy-3′-methyl- [2,2′-bipyridine]-5- carboxamide | 456 |
| Example 29 (A29) | 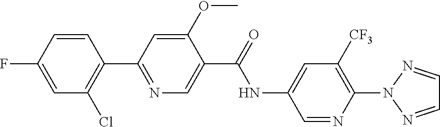 [SMILES: COc1cc(-c2ccc(F)cc2Cl)ncc1C(=O)Nc1cnc(-n2nccn2)c(C(F)(F)F)c1] | N-(6-(2H-1,2,3-triazol-2- yl)-5- (trifluoromethyl)pyridin-3- yl)-6-(2-chloro-4- fluorophenyl)-4- methoxynicotinamide | 493 |
| Example 30 (A30) | 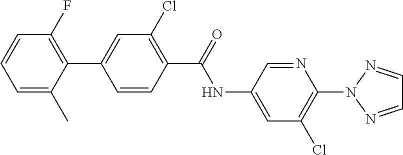 [SMILES: Cc1cccc(F)c1-c1ccc(C(=O)Nc2cnc(-n3nccn3)c(Cl)c2)c(Cl)c1] | 3-chloro-N-(5-chloro-6- (2H-1,2,3-triazol-2- yl)pyridin-3-yl)-2′-fluoro- 6′-methyl-[1,1′-biphenyl]- 4-carboxamide | 442 |
| Example 31 (A31) | 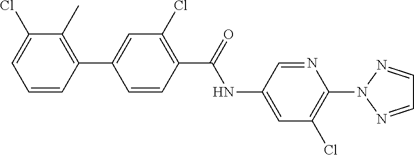 [SMILES: Cc1c(Cl)cccc1-c1ccc(C(=O)Nc2cnc(-n3nccn3)c(Cl)c2)c(Cl)c1] | 3,3′-dichloro-N-(5-chloro- 6-(2H-1,2,3-triazol-2- yl)pyridin-3-yl)-2′-methyl- [1,1′-biphenyl]-4- carboxamide | 458 |
| Example 32 (A32) | 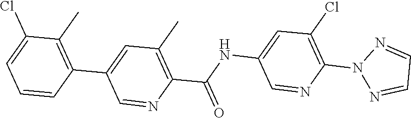 [SMILES: Cc1cc(-c2cccc(Cl)c2C)cnc1C(=O)Nc1cnc(-n2nccn2)c(Cl)c1] | 5-(3-chloro-2- methylphenyl)-N-(5- chloro-6-(2H-1,2,3-triazol- 2-yl)pyridin-3-yl)-3- methylpicolinamide | 439 |
| Example 33 (A33) | 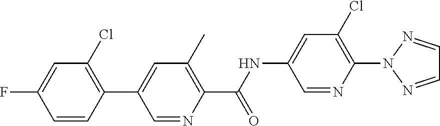 [SMILES: Cc1cc(-c2ccc(F)cc2Cl)cnc1C(=O)Nc1cnc(-n2nccn2)c(Cl)c1] | 5-(2-chloro-4- fluorophenyl)-N-(5-chloro- 6-(2H-1,2,3-triazol-2- yl)pyridin-3-yl)-3- methylpicolinamide | 443 |
| Example 34 (A34) | 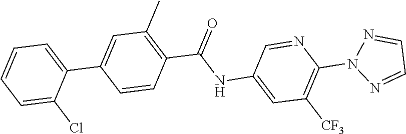 [SMILES: Cc1cc(-c2ccccc2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-chloro-3-methyl- [1,1′-biphenyl]-4- carboxamide | 458 |
| Example 36 (A36) | 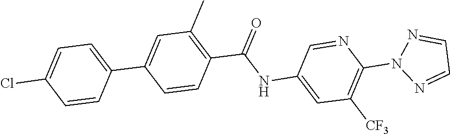 [SMILES: Cc1cc(-c2ccc(Cl)cc2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4′-chloro-3-methyl- [1,1′-biphenyl]-4- carboxamide | 458 |
| Example 37 (A37) | 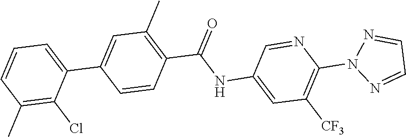 [SMILES: Cc1cc(-c2cccc(C)c2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-chloro-3,3′-dimethyl- [1,1′-biphenyl]-4- carboxamide | 472 |
| Example 38 (A38) | 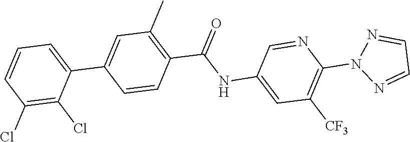 [SMILES: Cc1cc(-c2cccc(Cl)c2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′,3′-dichloro-3-methyl- [1,1′-biphenyl]-4- carboxamide | 492 |
| Example 39 (A39) | 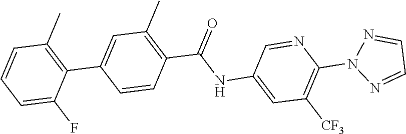 [SMILES: Cc1cc(-c2c(C)cccc2F)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-fluoro-3,6′-dimethyl- [1,1′-biphenyl]-4- carboxamide | 456 |
| Example 40 (A40) | 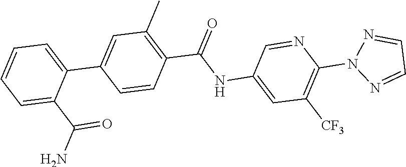 [SMILES: Cc1cc(-c2ccccc2C(N)=O)ccc1C(=O)Nc1cnc(-n2nccn2)c(C(F)(F)F)c1] | N4′-(6-(2H-1,2,3-triazol-2- yl)-5- (trifluoromethyl)pyridin-3- yl)-3′-methyl-[1,1′- biphenyl]-2,4′- dicarboxamide | 467 |
| Example 43 (A43) | 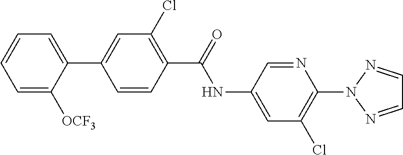 [SMILES: O=C(Nc1cnc(-n2nccn2)c(Cl)c1)c1ccc(-c2ccccc2OC(F)(F)F)cc1Cl] | 3-chloro-N-(5-chloro-6- (2H-1,2,3-triazol-2- yl)pyridin-3-yl)-2′- (trifluoromethoxy)-[1,1′- biphenyl]-4-carboxamide | 494 |
| Example 44 (A44) | 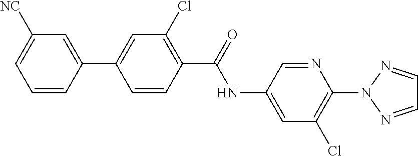 [SMILES: N#Cc1cccc(-c2ccc(C(=O)Nc3cnc(-n4nccn4)c(Cl)c3)c(Cl)c2)c1] | 3-chloro-N-(5-chloro-6- (2H-1,2,3-triazol-2- yl)pyridin-3-yl)-3′-cyano- [1,1′-biphenyl]-4- carboxamide | 435 |
| Example 45 (A45) | 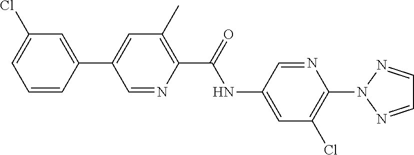 [SMILES: Cc1cc(-c2cccc(Cl)c2)cnc1C(=O)Nc1cnc(-n2nccn2)c(Cl)c1] | N-(5-chloro-6-(2H-1,2,3- triazol-2-yl)pyridin-3-yl)-5- (3-chlorophenyl)-3- methylpicolinamide | 425 |
| Example 46 (A46) | 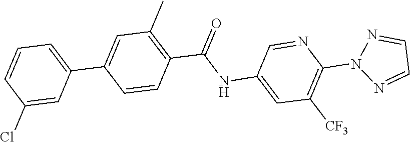 [SMILES: Cc1cc(-c2cccc(Cl)c2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-3′-chloro-3-methyl- [1,1′-biphenyl]-4- carboxamide | 458 |
| Example 47 (A47) | 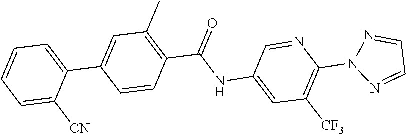 [SMILES: Cc1cc(-c2ccccc2C#N)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-cyano-3-methyl- [1,1′-biphenyl]-4- carboxamide | 449 |
| Example 48 (A48) | 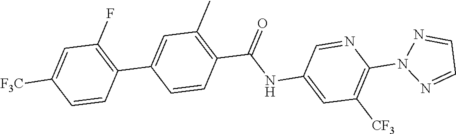 [SMILES: Cc1cc(-c2ccc(C(F)(F)F)cc2F)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-fluoro-3-methyl-4′- (trifluoromethyl)-[1,1′- biphenyl]-4-carboxamide | 510 |
| Example 49 (A49) | 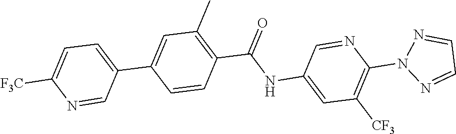 [SMILES: Cc1cc(-c2ccc(C(F)(F)F)nc2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2-methyl-4-(6- (trifluoromethyl)pyridin-3- yl)benzamide | 493 |
| Example 50 (A50) | 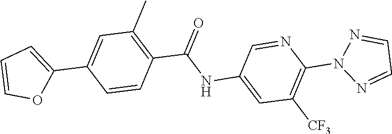 [SMILES: Cc1cc(-c2ccco2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4-(furan-2-yl)-2- methylbenzamide | 414 |
| Example 51 (A51) | 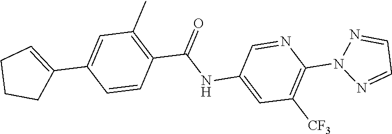 [SMILES: Cc1cc(C2=CCCC2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4-(cyclopent-1-en-1- yl)-2-methylbenzamide | 414 |
| Example 52 (A52) | 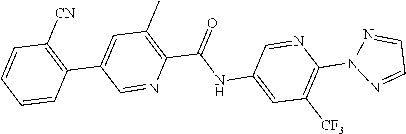 [SMILES: Cc1cc(-c2ccccc2C#N)cnc1C(=O)Nc1cnc(-n2nccn2)c(C(F)(F)F)c1] | N-(6-(2H-1,2,3-triazol-2- yl)-5- (trifluoromethyl)pyridin-3- yl)-5-(2-cyanophenyl)-3- methylpicolinamide | 450 |
| Example 53 (A53) | 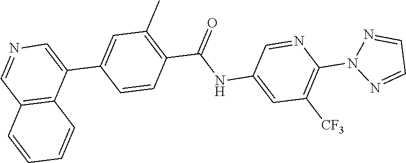 [SMILES: Cc1cc(-c2cncc3ccccc23)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4-(isoquinolin-4-yl)-2- methylbenzamide | 475 |
| Example 54 (A54) | 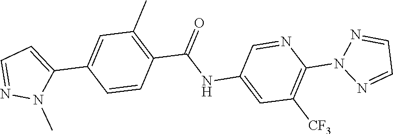 [SMILES: Cc1cc(-c2ccnn2C)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2-methyl-4-(1-methyl- 1H-pyrazol-5-yl)benzamide | 428 |
| Example 55 (A55) | 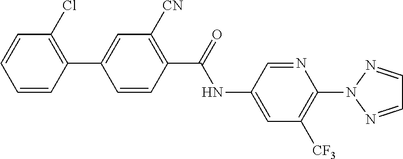 [SMILES: N#Cc1cc(-c2ccccc2Cl)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-chloro-3-cyano-[1,1′- biphenyl]-4-carboxamide | 469 |
| Example 56 (A56) | 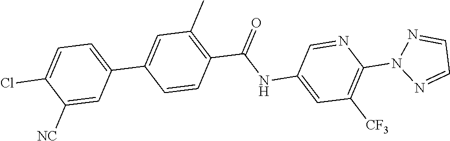 [SMILES: Cc1cc(-c2ccc(Cl)c(C#N)c2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4′-chloro-3′-cyano-3- methyl-[1,1′-biphenyl]-4- carboxamide | 483 |
| Example 57 (A57) | 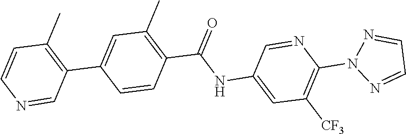 [SMILES: Cc1cc(-c2cnccc2C)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2-methyl-4-(4- methylpyridin-3- yl)benzamide | 439 |
| Example 58 (A58) | 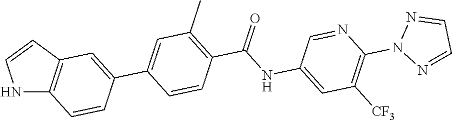 [SMILES: Cc1cc(-c2ccc3[nH]ccc3c2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4-(1H-indol-5-yl)-2- methylbenzamide | 463 |
| Example 59 (A59) | 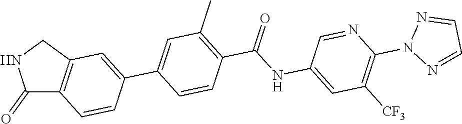 [SMILES: Cc1cc(-c2ccc3c(c2)CNC3=O)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2-methyl-4-(1- oxoisoindolin-5- yl)benzamide | 479 |
| Example 60 (A60) | 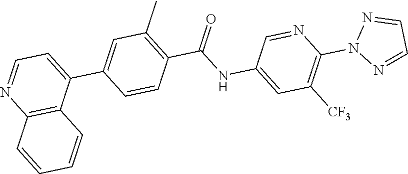 [SMILES: Cc1cc(-c2ccnc3ccccc23)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2-methyl-4-(quinolin-4- yl)benzamide | 475 |
| Example 61 (A61) | 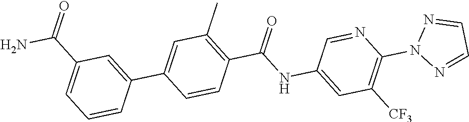 [SMILES: Cc1cc(-c2cccc(C(N)=O)c2)ccc1C(=O)Nc1cnc(-n2nccn2)c(C(F)(F)F)c1] | N4′-(6-(2H-1,2,3-triazol-2- yl)-5- (trifluoromethyl)pyridin-3- yl)-3′-methyl-[1,1′- biphenyl]-3,4′- dicarboxamide | 467 |
| Example 62 (A62) | 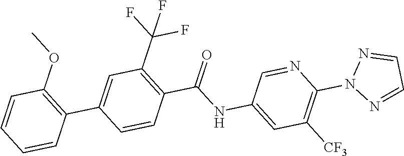 [SMILES: COc1ccccc1-c1ccc(C(=O)Nc2cnc(-n3nccn3)c(C(F)(F)F)c2)c(C(F)(F)F)c1] | N-(6-(2H-1,2,3-triazol-2- yl)-5- (trifluoromethyl)pyridin-3- yl)-2′-methoxy-3- (trifluoromethyl)-[1,1′- biphenyl]-4-carboxamide | 508 |
| Example 63 (A63) | 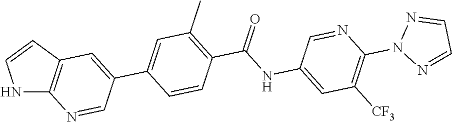 [SMILES: Cc1cc(-c2cnc3[nH]ccc3c2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2-methyl-4-(1H- pyrrolo[2,3-b]pyridin-5- yl)benzamide | 464 |
| Example 65 (A65) | 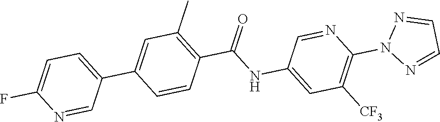 [SMILES: Cc1cc(-c2ccc(F)nc2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4-(6-fluoropyridin-3- yl)-2-methylbenzamide | 443 |
| Example 66 (A66) | 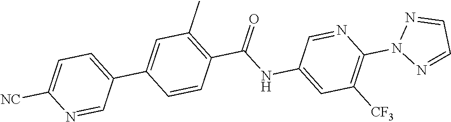 [SMILES: Cc1cc(-c2ccc(C#N)nc2)ccc1C(=O)Nc1cnc(-n2nccn2)c(C(F)(F)F)c1] | N-(6-(2H-1,2,3-triazol-2- yl)-5- (trifluoromethyl)pyridin-3- yl)-4-(6-cyanopyridin-3- yl)-2-methylbenzamide | 450 |
| Example 68 (A68) | 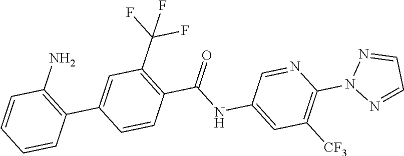 [SMILES: Nc1ccccc1-c1ccc(C(=O)Nc2cnc(-n3nccn3)c(C(F)(F)F)c2)c(C(F)(F)F)c1] | N-(6-(2H-1,2,3-triazol-2- yl)-5- (trifluoromethyl)pyridin-3- yl)-2′-amino-3- (trifluoromethyl)-[1,1′- biphenyl]-4-carboxamide | 493 |
| Example 70 (A70) | 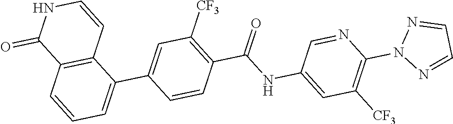 [SMILES: O=C(Nc1cnc(-n2nccn2)c(C(F)(F)F)c1)c1ccc(-c2cccc3c(=O)[nH]ccc23)cc1C(F)(F)F] | N-(6-(2H-1,2,3-triazol-2- yl)-5- (trifluoromethyl)pyridin-3- yl)-4-(1-oxo-1,2- dihydroisoquinolin-5-yl)-2- (trifluoromethyl)benzamide | 545 |
| Example 71 (A71) | 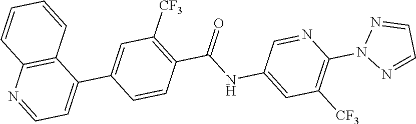 [SMILES: O=C(Nc1cnc(-n2nccn2)c(C(F)(F)F)c1)c1ccc(-c2ccnc3ccccc23)cc1C(F)(F)F] | N-(6-(2H-1,2,3-triazol-2- yl)-5- (trifluoromethyl)pyridin-3- yl)-4-(quinolin-4-yl)-2- (trifluoromethyl)benzamide | 529 |
| Example 72 (A72) | 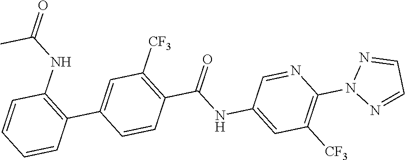 [SMILES: CC(=O)Nc1ccccc1-c1ccc(C(=O)Nc2cnc(-n3nccn3)c(C(F)(F)F)c2)c(C(F)(F)F)c1] | N-(6-(2H-1,2,3-triazol-2- yl)-5- (trifluoromethyl)pyridin-3- yl)-2′-acetamido-3- (trifluoromethyl)-[1,1′- biphenyl]-4-carboxamide | 535 |
| Example 73 (A73) | 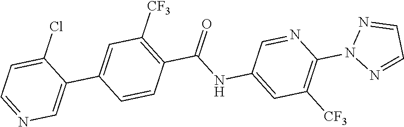 [SMILES: O=C(Nc1cnc(-n2nccn2)c(C(F)(F)F)c1)c1ccc(-c2cnccc2Cl)cc1C(F)(F)F] | N-(6-(2H-1,2,3-triazol-2- yl)-5- (trifluoromethyl)pyridin-3- yl)-4-(4-chloropyridin-3- yl)-2- (trifluoromethyl)benzamide | 513 |
| Example 74 (A74) | 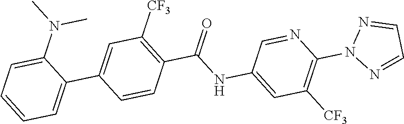 [SMILES: CN(C)c1ccccc1-c1ccc(C(=O)Nc2cnc(-n3nccn3)c(C(F)(F)F)c2)c(C(F)(F)F)c1] | N-(6-(2H-1,2,3-triazol-2- yl)-5- (trifluoromethyl)pyridin-3- yl)-2′-(dimethylamino)-3- (trifluoromethyl)-[1,1′- biphenyl]-4-carboxamide | 521 |
| Example 75 (A75) | 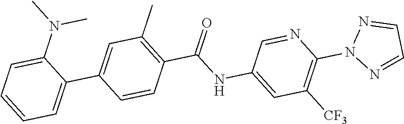 [SMILES: Cc1cc(-c2ccccc2N(C)C)ccc1C(=O)Nc1cnc(-n2nccn2)c(C(F)(F)F)c1] | N-(6-(2H-1,2,3-triazol-2- yl)-5- (trifluoromethyl)pyridin-3- yl)-2′-(dimethylamino)-3- methyl-[1,1′-biphenyl]-4- carboxamide | 467 |
| Example 76 (A76) | 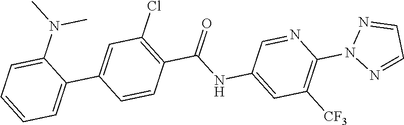 [SMILES: CN(C)c1ccccc1-c1ccc(C(=O)Nc2cnc(-n3nccn3)c(C(F)(F)F)c2)c(Cl)c1] | N-(6-(2H-1,2,3-triazol-2- yl)-5- (trifluoromethyl)pyridin-3- yl)-3-chloro-2′- (dimethylamino)-[1,1′- biphenyl]-4-carboxamide | 487 |
| Example 77 (A77) | 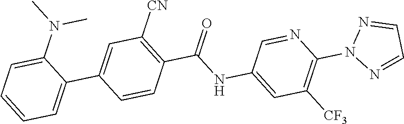 [SMILES: CN(C)c1ccccc1-c1ccc(C(=O)Nc2cnc(-n3nccn3)c(C(F)(F)F)c2)c(C#N)c1] | N-(6-(2H-1,2,3-triazol-2- yl)-5- (trifluoromethyl)pyridin-3- yl)-3-cyano-2′- (dimethylamino)-[1,1′- biphenyl]-4-carboxamide | 478 |
| Example 78 (A78) | 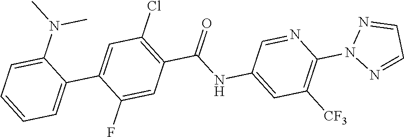 [SMILES: CN(C)c1ccccc1-c1cc(Cl)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-5-chloro-2′- (dimethylamino)-2-fluoro- [1,1′-biphenyl]-4- carboxamide | 505 |
| Example 79 (A79) | 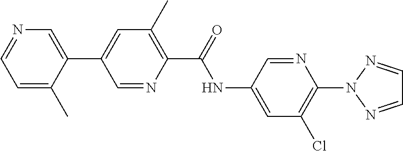 [SMILES: Cc1ccncc1-c1cnc(C(=O)Nc2cnc(-n3nccn3)c(Cl)c2)c(C)c1] | N-(5-chloro-6-(2H-1,2,3- triazol-2-yl)pyridin-3-yl)- 4′,5-dimethyl-[3,3′- bipyridine]-6-carboxamide | 406 |
| Example 80 (A80) | 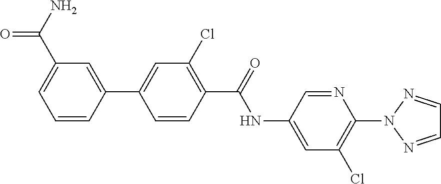 [SMILES: NC(=O)c1cccc(-c2ccc(C(=O)Nc3cnc(-n4nccn4)c(Cl)c3)c(Cl)c2)c1] | 3′-chloro-N4′-(5-chloro-6- (2H-1,2,3-triazol-2- yl)pyridin-3-yl)-[1,1′- biphenyl]-3,4′- dicarboxamide | 453 |
| Example 81 (A81) | 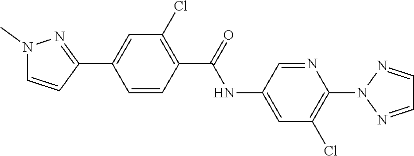 [SMILES: Cn1ccc(-c2ccc(C(=O)Nc3cnc(-n4nccn4)c(Cl)c3)c(Cl)c2)n1] | 2-chloro-N-(5-chloro-6- (2H-1,2,3-triazol-2- yl)pyridin-3-yl)-4-(1- methyl-1H-pyrazol-3- yl)benzamide | 414 |
| Example 82 (A82) | 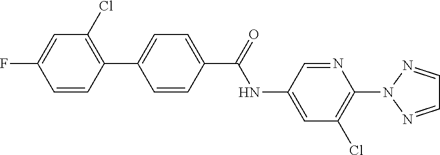 [SMILES: O=C(Nc1cnc(-n2nccn2)c(Cl)c1)c1ccc(-c2ccc(F)cc2Cl)cc1] | 2′-chloro-N-(5-chloro-6- (2H-1,2,3-triazol-2- yl)pyridin-3-yl)-4′-fluoro- [1,1′-biphenyl]-4- carboxamide | 428 |
| Example 83 (A83) | 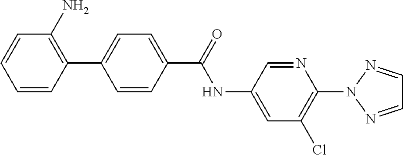 [SMILES: Nc1ccccc1-c1ccc(C(=O)Nc2cnc(-n3nccn3)c(Cl)c2)cc1] | 2′-amino-N-(5-chloro-6- (2H-1,2,3-triazol-2- yl)pyridin-3-yl)-[1,1′- biphenyl]-4-carboxamide | 391 |
| Example 84 (A84) | 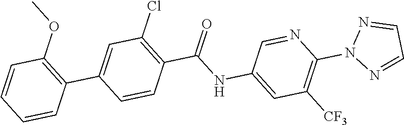 [SMILES: COc1ccccc1-c1ccc(C(=O)Nc2cnc(-n3nccn3)c(C(F)(F)F)c2)c(Cl)c1] | N-(6-(2H-1,2,3-triazol-2- yl)-5- (trifluoromethyl)pyridin-3- yl)-3-chloro-2′-methoxy- [1,1′-biphenyl]-4- carboxamide | 474 |
| Example 85 (A85) | 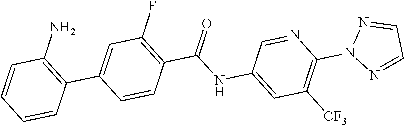 [SMILES: Nc1ccccc1-c1ccc(C(=O)Nc2cnc(-n3nccn3)c(C(F)(F)F)c2)c(F)c1] | N-(6-(2H-1,2,3-triazol-2- yl)-5- (trifluoromethyl)pyridin-3- yl)-2′-amino-3-fluoro-[1,1′- biphenyl]-4-carboxamide | 443 |
| Example 86 (A86) | 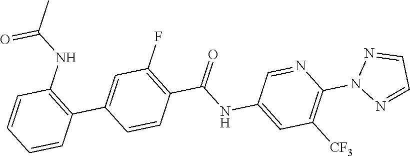 [SMILES: CC(=O)Nc1ccccc1-c1ccc(C(=O)Nc2cnc(-n3nccn3)c(C(F)(F)F)c2)c(F)c1] | N-(6-(2H-1,2,3-triazol-2- yl)-5- (trifluoromethyl)pyridin-3- yl)-2′-acetamido-3-fluoro- [1,1′-biphenyl]-4- carboxamide | 485 |
| Example 87 (A87) | 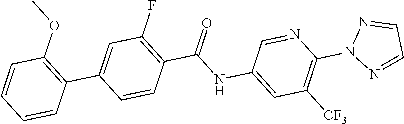 [SMILES: COc1ccccc1-c1ccc(C(=O)Nc2cnc(-n3nccn3)c(C(F)(F)F)c2)c(F)c1] | N-(6-(2H-1,2,3-triazol-2- yl)-5- (trifluoromethyl)pyridin-3- yl)-3-fluoro-2′-methoxy- [1,1′-biphenyl]-4- carboxamide | 458 |
| Example 88 (A88) | 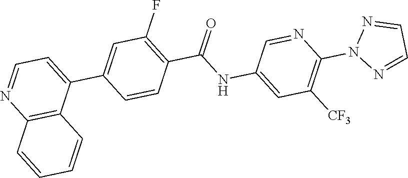 [SMILES: O=C(Nc1cnc(-n2nccn2)c(C(F)(F)F)c1)c1ccc(-c2ccnc3ccccc23)cc1F] | N-(6-(2H-1,2,3-triazol-2- yl)-5- (trifluoromethyl)pyridin-3- yl)-2-fluoro-4-(quinolin-4- yl)benzamide | 479 |
| Example 89 (A89) | 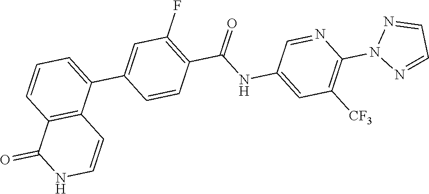 [SMILES: O=C(Nc1cnc(-n2nccn2)c(C(F)(F)F)c1)c1ccc(-c2cccc3c(=O)[nH]ccc23)cc1F] | N-(6-(2H-1,2,3-triazol-2- yl)-5- (trifluoromethyl)pyridin-3- yl)-2-fluoro-4-(1-oxo-1,2- dihydroisoquinolin-5- yl)benzamide | 495 |
| Example 90 (A90) | 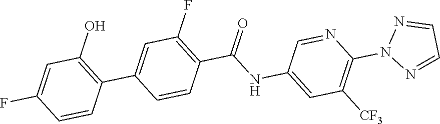 [SMILES: O=C(Nc1cnc(-n2nccn2)c(C(F)(F)F)c1)c1ccc(-c2ccc(F)cc2O)cc1F] | N-(6-(2H-1,2,3-triazol-2- yl)-5- (trifluoromethyl)pyridin-3- yl)-3,4′-difluoro-2′- hydroxy-[1,1′-biphenyl]-4- carboxamide | 462 |
| Example 91 (A91) | 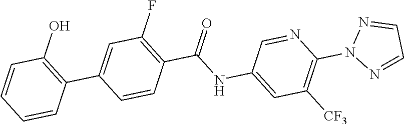 [SMILES: O=C(Nc1cnc(-n2nccn2)c(C(F)(F)F)c1)c1ccc(-c2ccccc2O)cc1F] | N-(6-(2H-1,2,3-triazol-2- yl)-5- (trifluoromethyl)pyridin-3- yl)-3-fluoro-2′-hydroxy- [1,1′-biphenyl]-4- carboxamide | 444 |
| Example 92 (A92) | 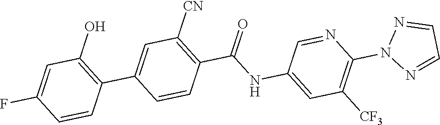 [SMILES: N#Cc1cc(-c2ccc(F)cc2O)ccc1C(=O)Nc1cnc(-n2nccn2)c(C(F)(F)F)c1] | N-(6-(2H-1,2,3-triazol-2- yl)-5- (trifluoromethyl)pyridin-3- yl)-3-cyano-4′-fluoro-2′- hydroxy-[1,1′-biphenyl]-4- carboxamide | 469 |
| Example 93 (A93) | 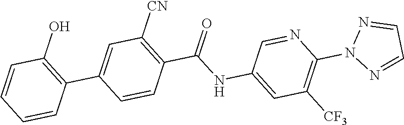 [SMILES: N#Cc1cc(-c2ccccc2O)ccc1C(=O)Nc1cnc(-n2nccn2)c(C(F)(F)F)c1] | N-(6-(2H-1,2,3-triazol-2- yl)-5- (trifluoromethyl)pyridin-3- yl)-3-cyano-2′-hydroxy- [1,1′-biphenyl]-4- carboxamide | 451 |
| Example 94 (A94) | 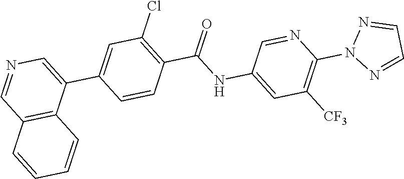 [SMILES: O=C(Nc1cnc(-n2nccn2)c(C(F)(F)F)c1)c1ccc(-c2cncc3ccccc23)cc1Cl] | N-(6-(2H-1,2,3-triazol-2- yl)-5- (trifluoromethyl)pyridin-3- yl)-2-chloro-4-(isoquinolin- 4-yl)benzamide | 495 |
| Example 95 (A95) | 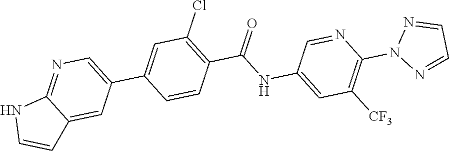 [SMILES: O=C(Nc1cnc(-n2nccn2)c(C(F)(F)F)c1)c1ccc(-c2cnc3[nH]ccc3c2)cc1Cl] | N-(6-(2H-1,2,3-triazol-2- yl)-5- (trifluoromethyl)pyridin-3- yl)-2-chloro-4-(1H- pyrrolo[2,3-b]pyridin-5- yl)benzamide | 484 |
| Example 96 (A96) | 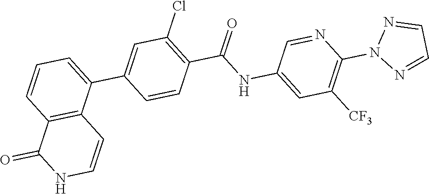 [SMILES: O=C(Nc1cnc(-n2nccn2)c(C(F)(F)F)c1)c1ccc(-c2cccc3c(=O)[nH]ccc23)cc1Cl] | N-(6-(2H-1,2,3-triazol-2- yl)-5- (trifluoromethyl)pyridin-3- yl)-2-chloro-4-(1-oxo-1,2- dihydroisoquinolin-5- yl)benzamide | 511 |
| Example 97 (A97) | 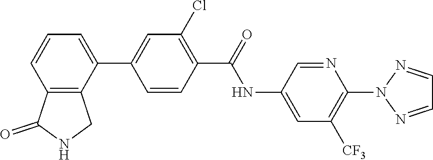 [SMILES: O=C(Nc1cnc(-n2nccn2)c(C(F)(F)F)c1)c1ccc(-c2cccc3c2CNC3=O)cc1Cl] | N-(6-(2H-1,2,3-triazol-2- yl)-5- (trifluoromethyl)pyridin-3- yl)-2-chloro-4-(1- oxoisoindolin-4- yl)benzamide | 499 |
| Example 99 (A99) | 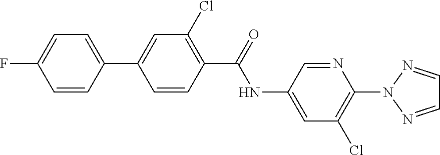 [SMILES: O=C(Nc1cnc(-n2nccn2)c(Cl)c1)c1ccc(-c2ccc(F)cc2)cc1Cl] | 3-chloro-N-(5-chloro-6- (2H-1,2,3-triazol-2- yl)pyridin-3-yl)-4′-fluoro- [1,1′-biphenyl]-4- carboxamide | 428 |
| Example 101 (A101) | 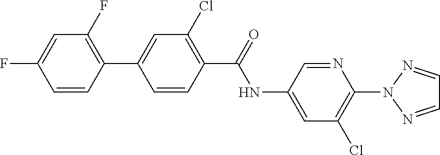 [SMILES: O=C(Nc1cnc(-n2nccn2)c(Cl)c1)c1ccc(-c2ccc(F)cc2F)cc1Cl] | 3-chloro-N-(5-chloro-6- (2H-1,2,3-triazol-2- yl)pyridin-3-yl)-2′,4′- difluoro-[1,1′-biphenyl]-4- carboxamide | 446 |
| Example 103 (A103) | 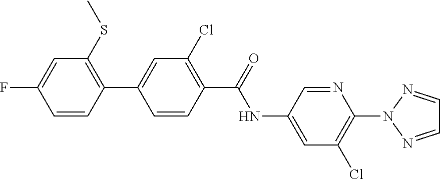 [SMILES: CSc1cc(F)ccc1-c1ccc(C(=O)Nc2cnc(-n3nccn3)c(Cl)c2)c(Cl)c1] | 3-chloro-N-(5-chloro-6- (2H-1,2,3-triazol-2- yl)pyridin-3-yl)-4′-fluoro- 2′-(methylthio)-[1,1′- biphenyl]-4-carboxamide | 474 |
| Example 106 (A106) | 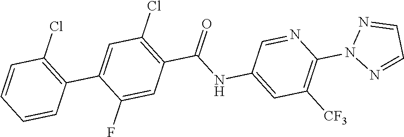 [SMILES: O=C(Nc1cnc(-n2nccn2)c(C(F)(F)F)c1)c1cc(F)c(-c2ccccc2Cl)cc1Cl] | N-(6-(2H-1,2,3-triazol-2- yl)-5- (trifluoromethyl)pyridin-3- yl)-2′,5-dichloro-2-fluoro- [1,1′-biphenyl]-4- carboxamide | 496 |
| Example 107 (A107) | 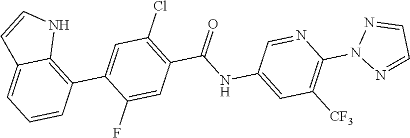 [SMILES: O=C(Nc1cnc(-n2nccn2)c(C(F)(F)F)c1)c1cc(F)c(-c2cccc3cc[nH]c23)cc1Cl] | N-(6-(2H-1,2,3-triazol-2- yl)-5- (trifluoromethyl)pyridin-3- yl)-2-chloro-5-fluoro-4- (1H-indol-7-yl)benzamide | 501 |
| Example 108 (A108) | 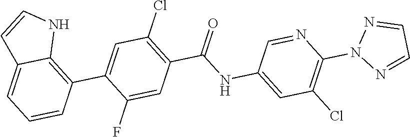 [SMILES: O=C(Nc1cnc(-n2nccn2)c(Cl)c1)c1cc(F)c(-c2cccc3cc[nH]c23)cc1Cl] | 2-chloro-N-(5-chloro-6- (2H-1,2,3-triazol-2- yl)pyridin-3-yl)-5-fluoro-4- (1H-indol-7-yl)benzamide | 467 |
| Example 109 (A109) | 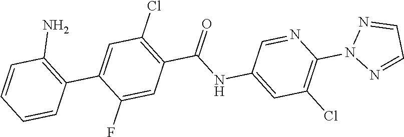 [SMILES: Nc1ccccc1-c1cc(Cl)c(C(=O)Nc2cnc(-n3nccn3)c(Cl)c2)cc1F] | 2′-amino-5-chloro-N-(5- chloro-6-(2H-1,2,3-triazol- 2-yl)pyridin-3-yl)-2-fluoro- [1,1′-biphenyl]-4- carboxamide | 443 |
| Example 111 (A111) | 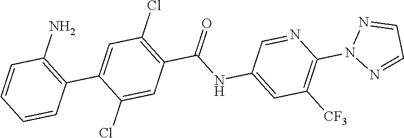 [SMILES: Nc1ccccc1-c1cc(Cl)c(C(=O)Nc2cnc(-n3nccn3)c(C(F)(F)F)c2)cc1Cl] | N-(6-(2H-1,2,3-triazol-2- yl)-5- (trifluoromethyl)pyridin-3- yl)-2′-amino-2,5-dichloro- [1,1′-biphenyl]-4- carboxamide | 493 |
| Example 112 (A112) | 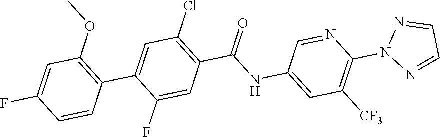 [SMILES: COc1cc(F)ccc1-c1cc(Cl)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-5-chloro-2,4′-difluoro- 2′-methoxy-[1,1′-biphenyl]- 4-carboxamide | 510 |
| Example 115 (A115) | 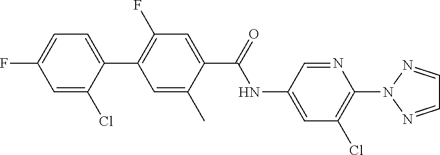 [SMILES: Cc1cc(-c2ccc(F)cc2Cl)c(F)cc1C(=O)Nc1cnc(-n2nccn2)c(Cl)c1] | 2′-chloro-N-(5-chloro-6- (2H-1,2,3-triazol-2- yl)pyridin-3-yl)-2,4′- difluoro-5-methyl-[1,1′- biphenyl]-4-carboxamide | 460 |
| Example 116 (A116) | 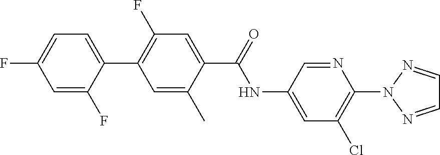 [SMILES: Cc1cc(-c2ccc(F)cc2F)c(F)cc1C(=O)Nc1cnc(-n2nccn2)c(Cl)c1] | N-(5-chloro-6-(2H-1,2,3- triazol-2-yl)pyridin-3-yl)- 2,2′,4′-trifluoro-5-methyl- [1,1′-biphenyl]-4- carboxamide | 444 |
| Example 117 (A117) | 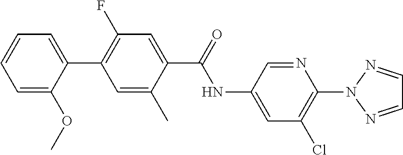 [SMILES: COc1ccccc1-c1cc(C)c(C(=O)Nc2cnc(-n3nccn3)c(Cl)c2)cc1F] | N-(5-chloro-6-(2H-1,2,3- triazol-2-yl)pyridin-3-yl)-2- fluoro-2′-methoxy-5- methyl-[1,1′-biphenyl]-4- carboxamide | 438 |
| Example 118 (A118) | 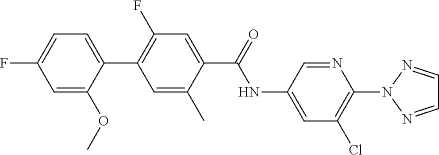 [SMILES: COc1cc(F)ccc1-c1cc(C)c(C(=O)Nc2cnc(-n3nccn3)c(Cl)c2)cc1F] | N-(5-chloro-6-(2H-1,2,3- triazol-2-yl)pyridin-3-yl)- 2,4′-difluoro-2′-methoxy-5- methyl-[1,1′-biphenyl]-4- carboxamide | 456 |
| Example 119 (A119) | 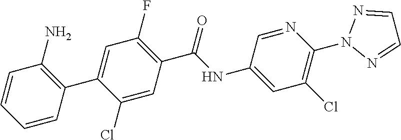 [SMILES: Nc1ccccc1-c1cc(F)c(C(=O)Nc2cnc(-n3nccn3)c(Cl)c2)cc1Cl] | 2′-amino-2-chloro-N-(5- chloro-6-(2H-1,2,3-triazol- 2-yl)pyridin-3-yl)-5-fluoro- [1,1′-biphenyl]-4- carboxamide | 443 |
| Example 120 (A120) | 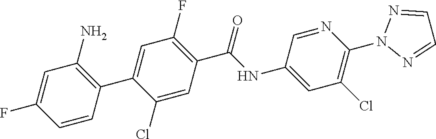 [SMILES: Nc1cc(F)ccc1-c1cc(F)c(C(=O)Nc2cnc(-n3nccn3)c(Cl)c2)cc1Cl] | 2′-amino-2-chloro-N-(5- chloro-6-(2H-1,2,3-triazol- 2-yl)pyridin-3-yl)-4′,5- difluoro-[1,1′-biphenyl]-4- carboxamide | 461 |
| Example 121 (A121) | 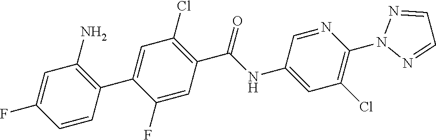 [SMILES: Nc1cc(F)ccc1-c1cc(Cl)c(C(=O)Nc2cnc(-n3nccn3)c(Cl)c2)cc1F] | 2′-amino-5-chloro-N-(5- chloro-6-(2H-1,2,3-triazol- 2-yl)pyridin-3-yl)-2,4′- difluoro-[1,1′-biphenyl]-4- carboxamide | 461 |
| Example 122 (A122) | 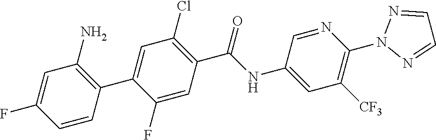 [SMILES: Nc1cc(F)ccc1-c1cc(Cl)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-2′-amino-5-chloro-2,4′- difluoro-[1,1′-biphenyl]-4- carboxamide | 495 |
| Example 123 (A123) | 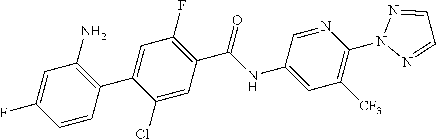 [SMILES: Nc1cc(F)ccc1-c1cc(F)c(C(=O)Nc2cnc(-n3nccn3)c(C(F)(F)F)c2)cc1Cl] | N-(6-(2H-1,2,3-triazol-2- yl)-5- (trifluoromethyl)pyridin-3- yl)-2′-amino-2-chloro-4′,5- difluoro-[1,1′-biphenyl]-4- carboxamide | 495 |
| Example 125 (A125) | 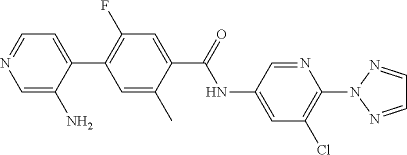 [SMILES: Cc1cc(-c2ccncc2N)c(F)cc1C(=O)Nc1cnc(-n2nccn2)c(Cl)c1] | 4-(3-aminopyridin-4-yl)-N- (5-chloro-6-(2H-1,2,3- triazol-2-yl)pyridin-3-yl)-5- fluoro-2-methylbenzamide | 424 |
| Example 127 (A127) | 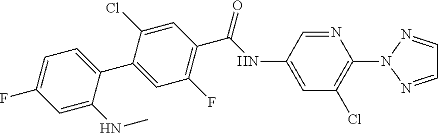 [SMILES: CNc1cc(F)ccc1-c1cc(F)c(C(=O)Nc2cnc(-n3nccn3)c(Cl)c2)cc1Cl] | 2-chloro-N-(5-chloro-6- (2H-1,2,3-triazol-2- yl)pyridin-3-yl)-4′,5- difluoro-2′-(methylamino)- [1,1′-biphenyl]-4- carboxamide | 475 |
| Example 128 (A128) | 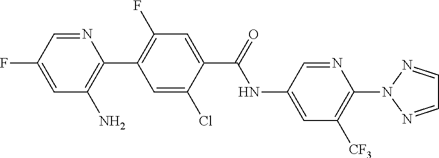 [SMILES: Nc1cc(F)cnc1-c1cc(Cl)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-4-(3-amino-5- fluoropyridin-2-yl)-2- chloro-5-fluorobenzamide | 496 |
| Example 129 (A129) | 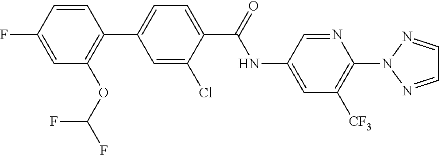 [SMILES: O=C(Nc1cnc(-n2nccn2)c(C(F)(F)F)c1)c1ccc(-c2ccc(F)cc2OC(F)F)cc1Cl] | N-(6-(2H-1,2,3-triazol-2- yl)-5- (trifluoromethyl)pyridin-3- yl)-3-chloro-2′- (difluoromethoxy)-4′- fluoro-[1,1′-biphenyl]-4- carboxamide | 528 |
| Example 131 (A131) | 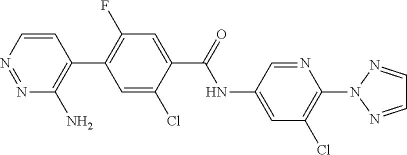 [SMILES: Nc1nnccc1-c1cc(Cl)c(C(=O)Nc2cnc(-n3nccn3)c(Cl)c2)cc1F] | 4-(3-aminopyridazin-4-yl)- 2-chloro-N-(5-chloro-6- (2H-1,2,3-triazol-2- yl)pyridin-3-yl)-5- fluorobenzamide | 445 |
| Example 132 (A132) | 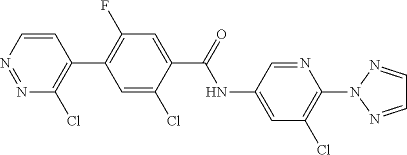 [SMILES: O=C(Nc1cnc(-n2nccn2)c(Cl)c1)c1cc(F)c(-c2ccnnc2Cl)cc1Cl] | 2-chloro-N-(5-chloro-6- (2H-1,2,3-triazol-2- yl)pyridin-3-yl)-4-(3- chloropyridazin-4-yl)-5- fluorobenzamide | 463 |
| Example 134 (A134) | 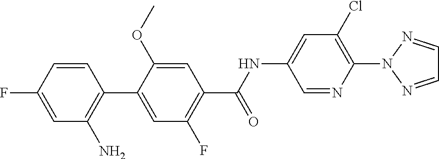 [SMILES: COc1cc(C(=O)Nc2cnc(-n3nccn3)c(Cl)c2)c(F)cc1-c1ccc(F)cc1N] | 2′-amino-N-(5-chloro-6- (2H-1,2,3-triazol-2- yl)pyridin-3-yl)-4′,5- difluoro-2-methoxy-[1,1′- biphenyl]-4-carboxamide | 457 |
| Example 135 (A135) | 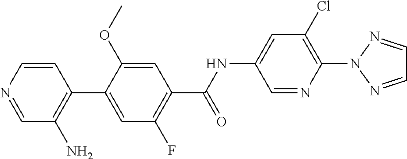 [SMILES: COc1cc(C(=O)Nc2cnc(-n3nccn3)c(Cl)c2)c(F)cc1-c1ccncc1N] | 4-(3-aminopyridin-4-yl)-N- (5-chloro-6-(2H-1,2,3- triazol-2-yl)pyridin-3-yl)-2- fluoro-5- methoxybenzamide | 440 |
| Example 136 (A136) | 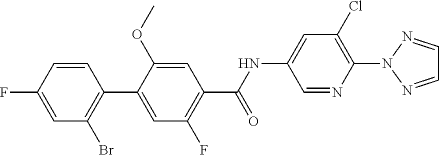 [SMILES: COc1cc(C(=O)Nc2cnc(-n3nccn3)c(Cl)c2)c(F)cc1-c1ccc(F)cc1Br] | 2′-bromo-N-(5-chloro-6- (2H-1,2,3-triazol-2- yl)pyridin-3-yl)-4′,5- difluoro-2-methoxy-[1,1′- biphenyl]-4-carboxamide | 519 |
| Example 137 (A137) | 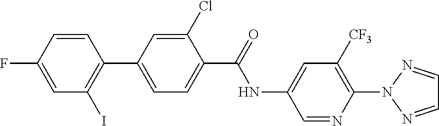 [SMILES: O=C(Nc1cnc(-n2nccn2)c(C(F)(F)F)c1)c1ccc(-c2ccc(F)cc2I)cc1Cl] | N-(6-(2H-1,2,3-triazol-2- yl)-5- (trifluoromethyl)pyridin-3- yl)-3-chloro-4′-fluoro-2′- iodo-[1,1′-biphenyl]-4- carboxamide | 387 |
| Example 138 (A138) | 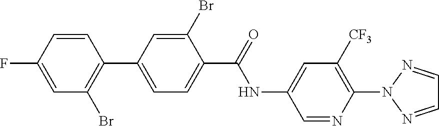 [SMILES: O=C(Nc1cnc(-n2nccn2)c(C(F)(F)F)c1)c1ccc(-c2ccc(F)cc2Br)cc1Br] | N-(6-(2H-1,2,3-triazol-2- yl)-5- (trifluoromethyl)pyridin-3- yl)-2′,3-dibromo-4′-fluoro- [1,1′-biphenyl]-4- carboxamide | 583 |
| Example 139 (A139) | 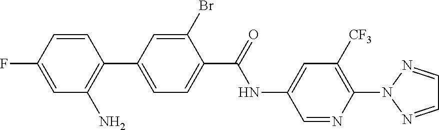 [SMILES: Nc1cc(F)ccc1-c1ccc(C(=O)Nc2cnc(-n3nccn3)c(C(F)(F)F)c2)c(Br)c1] | N-(6-(2H-1,2,3-triazol-2- yl)-5- (trifluoromethyl)pyridin-3- yl)-2′-amino-3-bromo-4′- fluoro-[1,1′-biphenyl]-4- carboxamide | 522 |
| Example 140 (A140) | 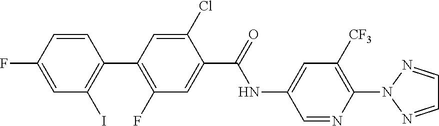 [SMILES: O=C(Nc1cnc(-n2nccn2)c(C(F)(F)F)c1)c1cc(F)c(-c2ccc(F)cc2I)cc1Cl] | N-(6-(2H-1,2,3-triazol-2- yl)-5- (trifluoromethyl)pyridin-3- yl)-5-chloro-2,4′-difluoro- 2′-iodo-[1,1′-biphenyl]-4- carboxamide | 605 |
| Example 141 (A141) | 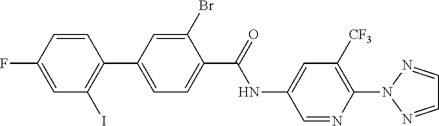 [SMILES: O=C(Nc1cnc(-n2nccn2)c(C(F)(F)F)c1)c1ccc(-c2ccc(F)cc2I)cc1Br] | N-(6-(2H-1,2,3-triazol-2- yl)-5- (trifluoromethyl)pyridin-3- yl)-3-bromo-4′-fluoro-2′- iodo-[1,1′-biphenyl]-4- carboxamide | 631 |
| Example 142 (A142) | 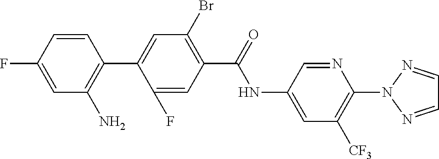 [SMILES: Nc1cc(F)ccc1-c1cc(Br)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-2′-amino-5-bromo-2,4′- difluoro-[1,1′-biphenyl]-4- carboxamide | 539 |
| Example 143 (A143) | 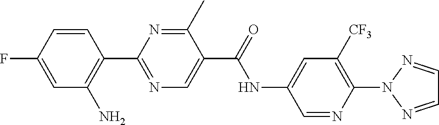 [SMILES: Cc1nc(-c2ccc(F)cc2N)ncc1C(=O)Nc1cnc(-n2nccn2)c(C(F)(F)F)c1] | N-(6-(2H-1,2,3-triazol-2- yl)-5- (trifluoromethyl)pyridin-3- yl)-2-(2-amino-4- fluorophenyl)-4- methylpyrimidine-5- carboxamide | 459 |
| Example 144 (A144) | 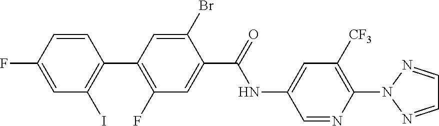 [SMILES: O=C(Nc1cnc(-n2nccn2)c(C(F)(F)F)c1)c1cc(F)c(-c2ccc(F)cc2I)cc1Br] | N-(6-(2H-1,2,3-triazol-2- yl)-5- (trifluoromethyl)pyridin-3- yl)-5-bromo-2,4′-difluoro- 2′-iodo-[1,1′-biphenyl]-4- carboxamide | 649 |
| Example 145 (A145) | 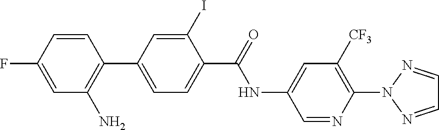 [SMILES: Nc1cc(F)ccc1-c1ccc(C(=O)Nc2cnc(-n3nccn3)c(C(F)(F)F)c2)c(I)c1] | N-(6-(2H-1,2,3-triazol-2- yl)-5- (trifluoromethyl)pyridin-3- yl)-2′-amino-4′-fluoro-3- iodo-[1,1′-biphenyl]-4- carboxamide | 569 |
| Example 146 (A146) | 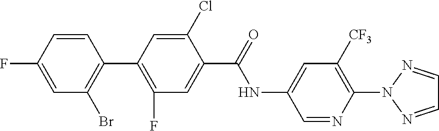 [SMILES: O=C(Nc1cnc(-n2nccn2)c(C(F)(F)F)c1)c1cc(F)c(-c2ccc(F)cc2Br)cc1Cl] | N-(6-(2H-1,2,3-triazol-2- yl)-5- (trifluoromethyl)pyridin-3- yl)-2′-bromo-5-chloro-2,4′- difluoro-[1,1′-biphenyl]-4- carboxamide | 557 |
| Example 148 (A148) | 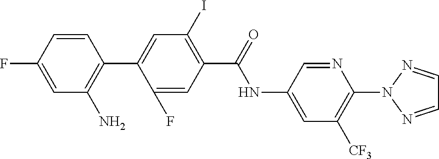 [SMILES: Nc1cc(F)ccc1-c1cc(I)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-2′-amino-2,4′-difluoro- 5-iodo-[1,1′-biphenyl]-4- carboxamide | 587 |
| Example 149 (A149) | 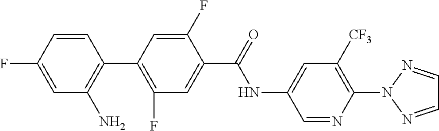 [SMILES: Nc1cc(F)ccc1-c1cc(F)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-2′-amino-2,4′,5- trifluoro-[1,1′-biphenyl]-4- carboxamide | 478 |
| Example 150 (A150) | 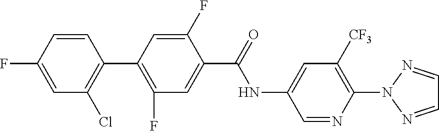 [SMILES: O=C(Nc1cnc(-n2nccn2)c(C(F)(F)F)c1)c1cc(F)c(-c2ccc(F)cc2Cl)cc1F] | N-(6-(2H-1,2,3-triazol-2- yl)-5- (trifluoromethyl)pyridin-3- yl)-2′-chloro-2,4′,5- trifluoro-[1,1′-biphenyl]-4- carboxamide | 498 |
| Example 151 (A151) | 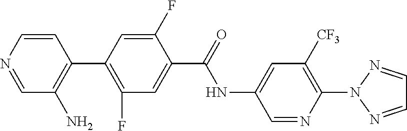 [SMILES: Nc1cnccc1-c1cc(F)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-4-(3-aminopyridin-4- yl)-2,5-difluorobenzamide | 462 |
| Example 152 (A152) | 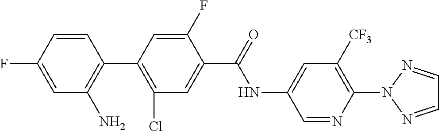 [SMILES: Nc1cc(F)ccc1-c1cc(F)c(C(=O)Nc2cnc(-n3nccn3)c(C(F)(F)F)c2)cc1Cl] | N-(6-(2H-1,2,3-triazol-2- yl)-5- (trifluoromethyl)pyridin-3- yl)-2′-amino-2-chloro-4′,5- difluoro-[1,1′-biphenyl]-4- carboxamide | 495 |
| Example 153 (A153) | 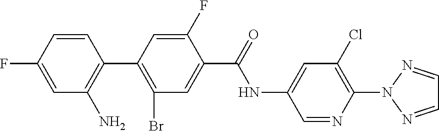 [SMILES: Nc1cc(F)ccc1-c1cc(F)c(C(=O)Nc2cnc(-n3nccn3)c(Cl)c2)cc1Br] | 2′-amino-2-bromo-N-(5- chloro-6-(2H-1,2,3-triazol- 2-yl)pyridin-3-yl)-4′,5- difluoro-[1,1′-biphenyl]-4- carboxamide | 504 |
| Example 154 (A154) | 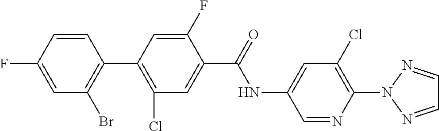 [SMILES: O=C(Nc1cnc(-n2nccn2)c(Cl)c1)c1cc(Cl)c(-c2ccc(F)cc2Br)cc1F] | 2′-bromo-2-chloro-N-(5- chloro-6-(2H-1,2,3-triazol- 2-yl)pyridin-3-yl)-4′,5- difluoro-[1,1′-biphenyl]-4- carboxamide | 523 |
| Example 155 (A155) | 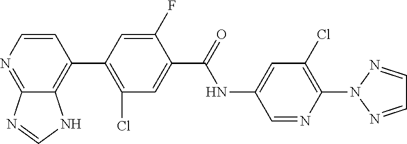 [SMILES: O=C(Nc1cnc(-n2nccn2)c(Cl)c1)c1cc(Cl)c(-c2ccnc3nc[nH]c23)cc1F] | 5-chloro-N-(5-chloro-6- (2H-1,2,3-triazol-2- yl)pyridin-3-yl)-2-fluoro-4- (1H-imidazo[4,5-b]pyridin- 7-yl)benzamide | 469 |
| Example 156 (A156) | 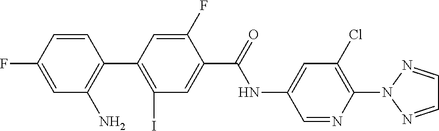 [SMILES: Nc1cc(F)ccc1-c1cc(F)c(C(=O)Nc2cnc(-n3nccn3)c(Cl)c2)cc1I] | 2′-amino-N-(5-chloro-6- (2H-1,2,3-triazol-2- yl)pyridin-3-yl)-4′,5- difluoro-2-iodo-[1,1′- biphenyl]-4-carboxamide | 552 |
| Example 158 (A158) | 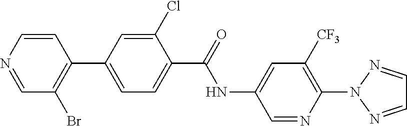 [SMILES: O=C(Nc1cnc(-n2nccn2)c(C(F)(F)F)c1)c1ccc(-c2ccncc2Br)cc1Cl] | N-(6-(2H-1,2,3-triazol-2- yl)-5- (trifluoromethyl)pyridin-3- yl)-4-(3-bromopyridin-4- yl)-2-chlorobenzamide | 522 |
| Example 159 (A159) | 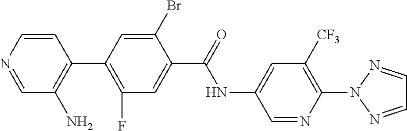 [SMILES: Nc1cnccc1-c1cc(Br)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-4-(3-aminopyridin-4- yl)-2-bromo-5- fluorobenzamide | 522 |
| Example 160 (A160) | 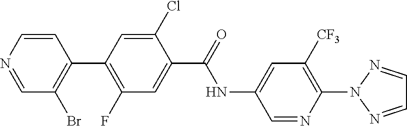 [SMILES: O=C(Nc1cnc(-n2nccn2)c(C(F)(F)F)c1)c1cc(F)c(-c2ccncc2Br)cc1Cl] | N-(6-(2H-1,2,3-triazol-2- yl)-5- (trifluoromethyl)pyridin-3- yl)-4-(3-bromopyridin-4- yl)-2-chloro-5- fluorobenzamide | 340 |
| Example 161 (A161) | 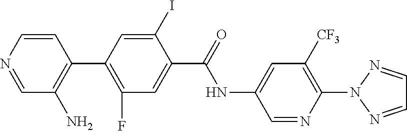 [SMILES: Nc1cnccc1-c1cc(I)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-4-(3-aminopyridin-4- yl)-5-fluoro-2- iodobenzamide | 570 |
| Example 162 (A162) | 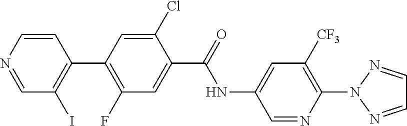 [SMILES: O=C(Nc1cnc(-n2nccn2)c(C(F)(F)F)c1)c1cc(F)c(-c2ccncc2I)cc1Cl] | N-(6-(2H-1,2,3-triazol-2- yl)-5- (trifluoromethyl)pyridin-3- yl)-2-chloro-5-fluoro-4-(3- iodopyridin-4- yl)benzamide | 588 |
| Example 163 (A163) | 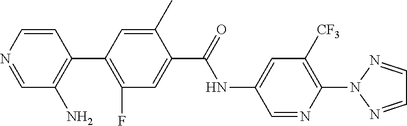 [SMILES: Cc1cc(-c2ccncc2N)c(F)cc1C(=O)Nc1cnc(-n2nccn2)c(C(F)(F)F)c1] | N-(6-(2H-1,2,3-triazol-2- yl)-5- (trifluoromethyl)pyridin-3- yl)-4-(3-aminopyridin-4- yl)-5-fluoro-2- methylbenzamide | 458 |
| Example 164 (A164) | 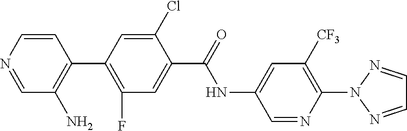 [SMILES: Nc1cnccc1-c1cc(Cl)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-4-(3-aminopyridin-4- yl)-2-chloro-5- fluorobenzamide | 478 |
| Example 165 (A165) | 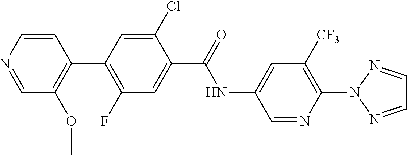 [SMILES: COc1cnccc1-c1cc(Cl)c(C(=O)Nc2cnc(-n3nccn3)c(C(F)(F)F)c2)cc1F] | N-(6-(2H-1,2,3-triazol-2- yl)-5- (trifluoromethyl)pyridin-3- yl)-2-chloro-5-fluoro-4-(3- methoxypyridin-4- yl)benzamide | 493 |
| Example 166 (A166) | 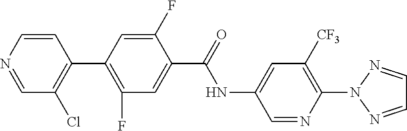 [SMILES: O=C(Nc1cnc(-n2nccn2)c(C(F)(F)F)c1)c1cc(F)c(-c2ccncc2Cl)cc1F] | N-(6-(2H-1,2,3-triazol-2- yl)-5- (trifluoromethyl)pyridin-3- yl)-4-(3-chloropyridin-4- yl)-2,5-difluorobenzamide | 481 |
| Example 167 (A167) | 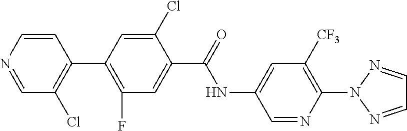 [SMILES: O=C(Nc1cnc(-n2nccn2)c(C(F)(F)F)c1)c1cc(F)c(-c2ccncc2Cl)cc1Cl] | N-(6-(2H-1,2,3-triazol-2- yl)-5- (trifluoromethyl)pyridin-3- yl)-2-chloro-4-(3- chloropyridin-4-yl)-5- fluorobenzamide | 497 |
| Example 168 (A168) | 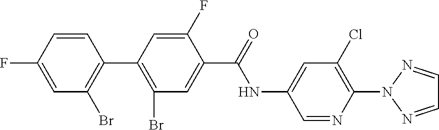 [SMILES: O=C(Nc1cnc(-n2nccn2)c(Cl)c1)c1cc(Br)c(-c2ccc(F)cc2Br)cc1F] | 2,2′-dibromo-N-(5-chloro- 6-(2H-1,2,3-triazol-2- yl)pyridin-3-yl)-4′,5- difluoro-[1,1′-biphenyl]-4- carboxamide | 567 |
| Example 169 (A169) | 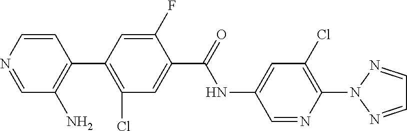 [SMILES: Nc1cnccc1-c1cc(F)c(C(=O)Nc2cnc(-n3nccn3)c(Cl)c2)cc1Cl] | 4-(3-aminopyridin-4-yl)-5- chloro-N-(5-chloro-6-(2H- 1,2,3-triazol-2-yl)pyridin- 3-yl)-2-fluorobenzamide | 444.05 |
| Example 170 (A170) | 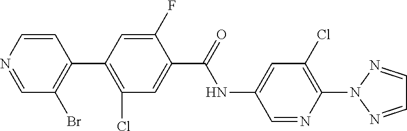 [SMILES: O=C(Nc1cnc(-n2nccn2)c(Cl)c1)c1cc(Cl)c(-c2ccncc2Br)cc1F] | 4-(3-bromopyridin-4-yl)-5- chloro-N-(5-chloro-6-(2H- 1,2,3-triazol-2-yl)pyridin- 3-yl)-2-fluorobenzamide | 506 |
| Example 171 (A171) | 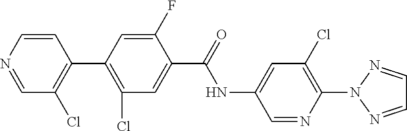 [SMILES: O=C(Nc1cnc(-n2nccn2)c(Cl)c1)c1cc(Cl)c(-c2ccncc2Cl)cc1F] | 5-chloro-N-(5-chloro-6- (2H-1,2,3-triazol-2- yl)pyridin-3-yl)-4-(3- chloropyridin-4-yl)-2- fluorobenzamide | 463 |
| Example 172 (A172) | 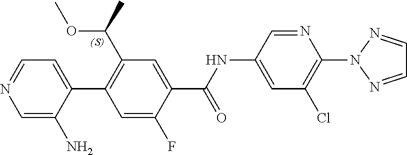 | (S)-4-(3-aminopyridin-4- yl)-N-(5-chloro-6-(2H- 1,2,3-triazol-2-yl)pyridin- 3-yl)-2-fluoro-5-(1- methoxyethyl)benzamide | 468 |
| Example 173 (A173) | 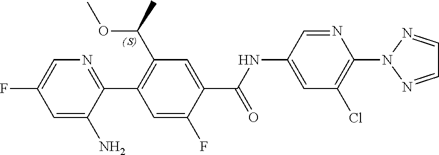 | (S)-4-(3-amino-5- fluoropyridin-2-yl)-N-(5- chloro-6-(2H-1,2,3-triazol- 2-yl)pyridin-3-yl)-2-fluoro- 5-(1- methoxyethyl)benzamide | 486 |
| Example 174 (A174) | 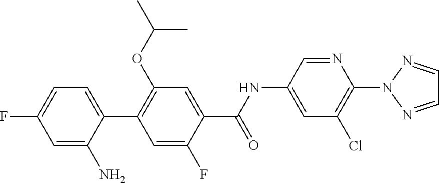 [SMILES: CC(C)Oc1cc(C(=O)Nc2cnc(-n3nccn3)c(Cl)c2)c(F)cc1-c1ccc(F)cc1N] | 2′-amino-N-(5-chloro-6- (2H-1,2,3-triazol-2- yl)pyridin-3-yl)-4′,5- difluoro-2-isopropoxy- [1,1′-biphenyl]-4- carboxamide | 485 |
| Example 175 (A175) | 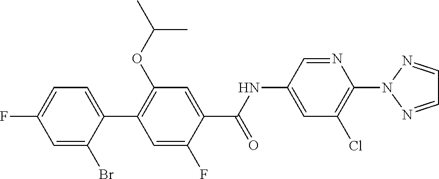 [SMILES: CC(C)Oc1cc(C(=O)Nc2cnc(-n3nccn3)c(Cl)c2)c(F)cc1-c1ccc(F)cc1Br] | 2′-bromo-N-(5-chloro-6- (2H-1,2,3-triazol-2- yl)pyridin-3-yl)-4′,5- difluoro-2-isopropoxy- [1,1′-biphenyl]-4- carboxamide | 548 |
| Example 176 (A176) | 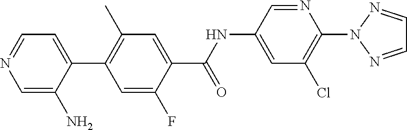 [SMILES: Cc1cc(C(=O)Nc2cnc(-n3nccn3)c(Cl)c2)c(F)cc1-c1ccncc1N] | 4-(3-aminopyridin-4-yl)-N- (5-chloro-6-(2H-1,2,3- triazol-2-yl)pyridin-3-yl)-2- fluoro-5-methylbenzamide | 424 |
| Example 177 (A177) | 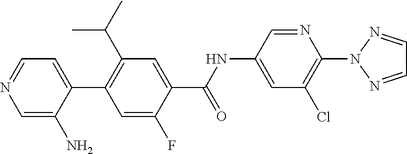 [SMILES: CC(C)c1cc(C(=O)Nc2cnc(-n3nccn3)c(Cl)c2)c(F)cc1-c1ccncc1N] | 4-(3-aminopyridin-4-yl)-N- (5-chloro-6-(2H-1,2,3- triazol-2-yl)pyridin-3-yl)-2- fluoro-5- isopropylbenzamide | 452 |
| Example 178 (A178) | 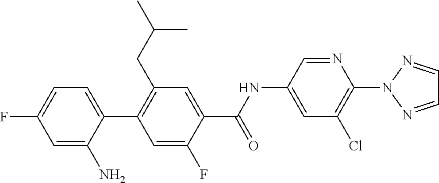 [SMILES: CC(C)Cc1cc(C(=O)Nc2cnc(-n3nccn3)c(Cl)c2)c(F)cc1-c1ccc(F)cc1N] | 2′-amino-N-(5-chloro-6- (2H-1,2,3-triazol-2- yl)pyridin-3-yl)-4′,5- difluoro-2-isobutyl-[1,1′- biphenyl]-4-carboxamide | 483 |
| Example 179 (A179) | 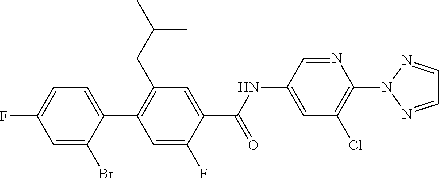 [SMILES: CC(C)Cc1cc(C(=O)Nc2cnc(-n3nccn3)c(Cl)c2)c(F)cc1-c1ccc(F)cc1Br] | 2′-bromo-N-(5-chloro-6- (2H-1,2,3-triazol-2- yl)pyridin-3-yl)-4′,5- difluoro-2-isobutyl-[1,1′- biphenyl]-4-carboxamide | 546 |
| Example 180 (A180) | 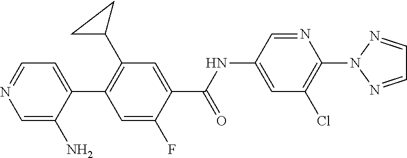 [SMILES: Nc1cnccc1-c1cc(F)c(C(=O)Nc2cnc(-n3nccn3)c(Cl)c2)cc1C1CC1] | 4-(3-aminopyridin-4-yl)-N- (5-chloro-6-(2H-1,2,3- triazol-2-yl)pyridin-3-yl)-5- cyclopropyl-2- fluorobenzamide | 450 |
| Example 181 (A181) | 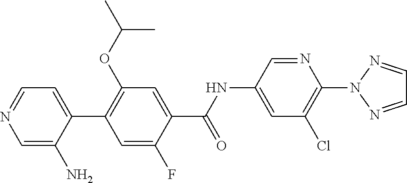 [SMILES: CC(C)Oc1cc(C(=O)Nc2cnc(-n3nccn3)c(Cl)c2)c(F)cc1-c1ccncc1N] | 4-(3-aminopyridin-4-yl)-N- (5-chloro-6-(2H-1,2,3- triazol-2-yl)pyridin-3-yl)-2- fluoro-5- isopropoxybenzamide | 468 |
| Example 182 (A182) | 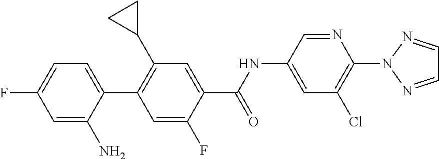 [SMILES: Nc1cc(F)ccc1-c1cc(F)c(C(=O)Nc2cnc(-n3nccn3)c(Cl)c2)cc1C1CC1] | 2′-amino-N-(5-chloro-6- (2H-1,2,3-triazol-2- yl)pyridin-3-yl)-2- cyclopropyl-4′,5-difluoro- [1,1′-biphenyl]-4- carboxamide | 467 |
| Example 183 (A183) | 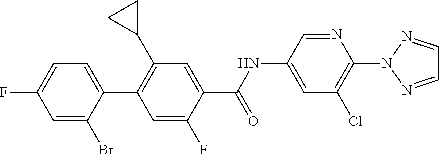 [SMILES: O=C(Nc1cnc(-n2nccn2)c(Cl)c1)c1cc(C2CC2)c(-c2ccc(F)cc2Br)cc1F] | 2′-bromo-N-(5-chloro-6- (2H-1,2,3-triazol-2- yl)pyridin-3-yl)-2- cyclopropyl-4′,5-difluoro- [1,1′-biphenyl]-4- carboxamide | 330 |
| Example 184 (A184) | 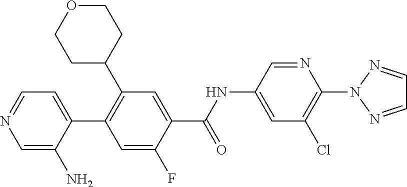 [SMILES: Nc1cnccc1-c1cc(F)c(C(=O)Nc2cnc(-n3nccn3)c(Cl)c2)cc1C1CCOCC1] | 4-(3-aminopyridin-4-yl)-N- (5-chloro-6-(2H-1,2,3- triazol-2-yl)pyridin-3-yl)-2- fluoro-5-(tetrahydro-2H- pyran-4-yl)benzamide | 494 |
| Example 185 (A185) | 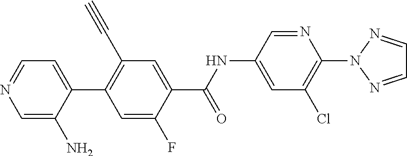 [SMILES: C#Cc1cc(C(=O)Nc2cnc(-n3nccn3)c(Cl)c2)c(F)cc1-c1ccncc1N] | 4-(3-aminopyridin-4-yl)-N- (5-chloro-6-(2H-1,2,3- triazol-2-yl)pyridin-3-yl)-5- ethynyl-2-fluorobenzamide | 434 |
| Example 186 (A186) | 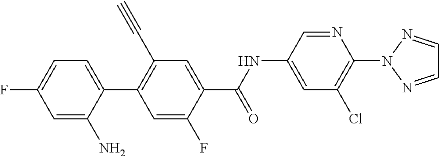 [SMILES: C#Cc1cc(C(=O)Nc2cnc(-n3nccn3)c(Cl)c2)c(F)cc1-c1ccc(F)cc1N] | 2′-amino-N-(5-chloro-6- (2H-1,2,3-triazol-2- yl)pyridin-3-yl)-2-ethynyl- 4′,5-difluoro-[1,1′- biphenyl]-4-carboxamide | 451 |
| Example 187 (A187) | 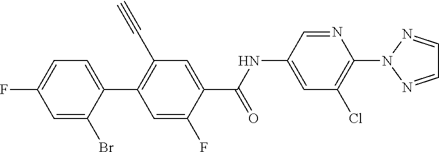 [SMILES: C#Cc1cc(C(=O)Nc2cnc(-n3nccn3)c(Cl)c2)c(F)cc1-c1ccc(F)cc1Br] | 2′-bromo-N-(5-chloro-6- (2H-1,2,3-triazol-2- yl)pyridin-3-yl)-2-ethynyl- 4′,5-difluoro-[1,1′- biphenyl]-4-carboxamide | 513 |
| Example 188 (A188) | 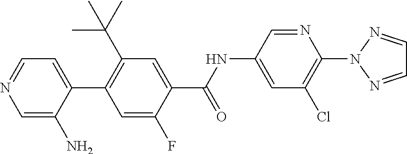 [SMILES: CC(C)(C)c1cc(C(=O)Nc2cnc(-n3nccn3)c(Cl)c2)c(F)cc1-c1ccncc1N] | 4-(3-aminopyridin-4-yl)-5- (tert-butyl)-N-(5-chloro-6- (2H-1,2,3-triazol-2- yl)pyridin-3-yl)-2- fluorobenzamide | 466 |
| Example 189 (A189) | 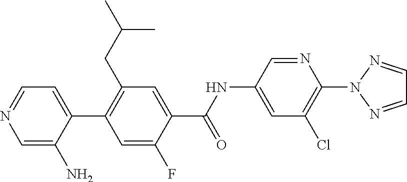 [SMILES: CC(C)Cc1cc(C(=O)Nc2cnc(-n3nccn3)c(Cl)c2)c(F)cc1-c1ccncc1N] | 4-(3-aminopyridin-4-yl)-N- (5-chloro-6-(2H-1,2,3- triazol-2-yl)pyridin-3-yl)-2- fluoro-5-isobutylbenzamide | 466 |
| Example 190 (A190) | 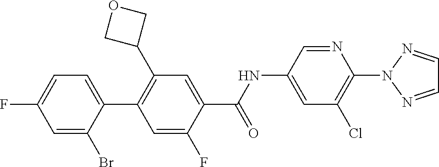 [SMILES: O=C(Nc1cnc(-n2nccn2)c(Cl)c1)c1cc(C2COC2)c(-c2ccc(F)cc2Br)cc1F] | 2′-bromo-N-(5-chloro-6- (2H-1,2,3-triazol-2- yl)pyridin-3-yl)-4′,5- difluoro-2-(oxetan-3-yl)- [1,1′-biphenyl]-4- carboxamide | 546 |
| Example 191 (A191) | 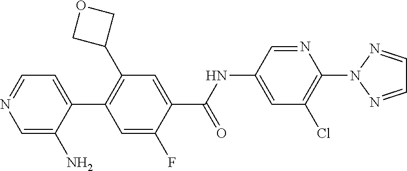 [SMILES: Nc1cnccc1-c1cc(F)c(C(=O)Nc2cnc(-n3nccn3)c(Cl)c2)cc1C1COC1] | 4-(3-aminopyridin-4-yl)-N- (5-chloro-6-(2H-1,2,3- triazol-2-yl)pyridin-3-yl)-2- fluoro-5-(oxetan-3- yl)benzamide | 466 |
| Example 192 (A192) | 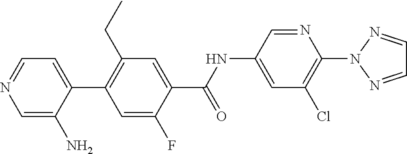 [SMILES: CCc1cc(C(=O)Nc2cnc(-n3nccn3)c(Cl)c2)c(F)cc1-c1ccncc1N] | 4-(3-aminopyridin-4-yl)-N- (5-chloro-6-(2H-1,2,3- triazol-2-yl)pyridin-3-yl)-5- ethyl-2-fluorobenzamide | 438 |
| Example 193 (A193) | 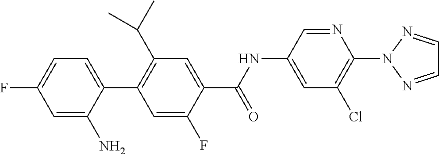 [SMILES: CC(C)c1cc(C(=O)Nc2cnc(-n3nccn3)c(Cl)c2)c(F)cc1-c1ccc(F)cc1N] | 2′-amino-N-(5-chloro-6- (2H-1,2,3-triazol-2- yl)pyridin-3-yl)-4′,5- difluoro-2-isopropyl-[1,1′- biphenyl]-4-carboxamide | 469 |
| Example 194 (A194) | 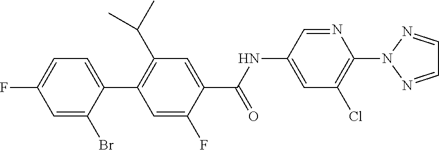 [SMILES: CC(C)c1cc(C(=O)Nc2cnc(-n3nccn3)c(Cl)c2)c(F)cc1-c1ccc(F)cc1Br] | 2′-bromo-N-(5-chloro-6- (2H-1,2,3-triazol-2- yl)pyridin-3-yl)-4′,5- difluoro-2-isopropyl-[1,1′- biphenyl]-4-carboxamide | 532 |
| Example 195 (A195) | 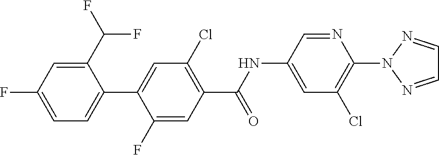 [SMILES: O=C(Nc1cnc(-n2nccn2)c(Cl)c1)c1cc(F)c(-c2ccc(F)cc2C(F)F)cc1Cl] | 5-chloro-N-(5-chloro-6- (2H-1,2,3-triazol-2- yl)pyridin-3-yl)-2′- (difluoromethyl)-2,4′- difluoro-[1,1′-biphenyl]-4- carboxamide | 496 |
| Example 196 (A196) | 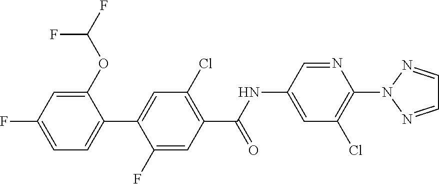 [SMILES: O=C(Nc1cnc(-n2nccn2)c(Cl)c1)c1cc(F)c(-c2ccc(F)cc2OC(F)F)cc1Cl] | 5-chloro-N-(5-chloro-6- (2H-1,2,3-triazol-2- yl)pyridin-3-yl)-2′- (difluoromethoxy)-2,4′- difluoro-[1,1′-biphenyl]-4- carboxamide | 512 |
| Example 197 (A197) | 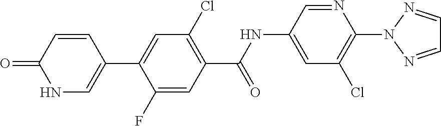 [SMILES: O=C(Nc1cnc(-n2nccn2)c(Cl)c1)c1cc(F)c(-c2ccc(=O)[nH]c2)cc1Cl] | 2-chloro-N-(5-chloro-6- (2H-1,2,3-triazol-2- yl)pyridin-3-yl)-5-fluoro-4- (6-oxo-1,6-dihydropyridin- 3-yl)benzamide | 445 |
| Example 198 (A198) | 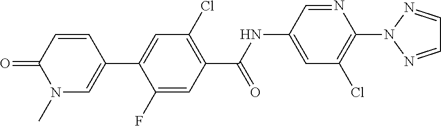 [SMILES: Cn1cc(-c2cc(Cl)c(C(=O)Nc3cnc(-n4nccn4)c(Cl)c3)cc2F)ccc1=O] | 2-chloro-N-(5-chloro-6- (2H-1,2,3-triazol-2- yl)pyridin-3-yl)-5-fluoro-4- (1-methyl-6-oxo-1,6- dihydropyridin-3- yl)benzamide | 459 |
| Example 199 (A199) | 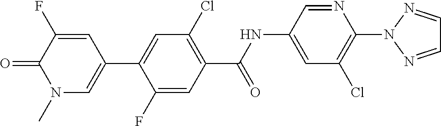 [SMILES: Cn1cc(-c2cc(Cl)c(C(=O)Nc3cnc(-n4nccn4)c(Cl)c3)cc2F)cc(F)c1=O] | 2-chloro-N-(5-chloro-6- (2H-1,2,3-triazol-2- yl)pyridin-3-yl)-5-fluoro-4- (5-fluoro-1-methyl-6-oxo- 1,6-dihydropyridin-3- yl)benzamide | 477 |
| Example 200 (A200) | 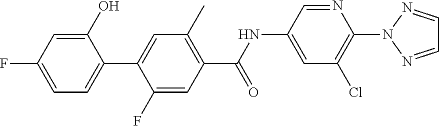 [SMILES: Cc1cc(-c2ccc(F)cc2O)c(F)cc1C(=O)Nc1cnc(-n2nccn2)c(Cl)c1] | N-(5-chloro-6-(2H-1,2,3- triazol-2-yl)pyridin-3-yl)- 2,4′-difluoro-2′-hydroxy-5- methyl-[1,1′-biphenyl]-4- carboxamide | 442 |
| Example 201 (A201) | 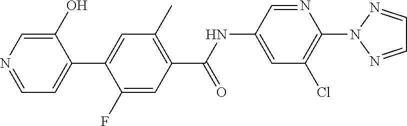 [SMILES: Cc1cc(-c2ccncc2O)c(F)cc1C(=O)Nc1cnc(-n2nccn2)c(Cl)c1] | N-(5-chloro-6-(2H-1,2,3- triazol-2-yl)pyridin-3-yl)-5- fluoro-4-(3- hydroxypyridin-4-yl)-2- methylbenzamide | 425 |
| Example 202 (A202) | 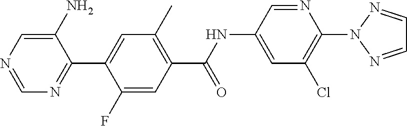 [SMILES: Cc1cc(-c2ncncc2N)c(F)cc1C(=O)Nc1cnc(-n2nccn2)c(Cl)c1] | 4-(5-aminopyrimidin-4-yl)- N-(5-chloro-6-(2H-1,2,3- triazol-2-yl)pyridin-3-yl)-5- fluoro-2-methylbenzamide | 425 |
| Example 203 (A203) | 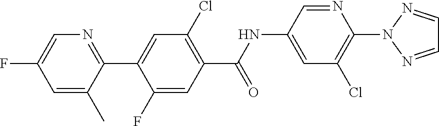 [SMILES: Cc1cc(F)cnc1-c1cc(Cl)c(C(=O)Nc2cnc(-n3nccn3)c(Cl)c2)cc1F] | 2-chloro-N-(5-chloro-6- (2H-1,2,3-triazol-2- yl)pyridin-3-yl)-5-fluoro-4- (5-fluoro-3-methylpyridin- 2-yl)benzamide | 461 |
| Example 204 (A204) | 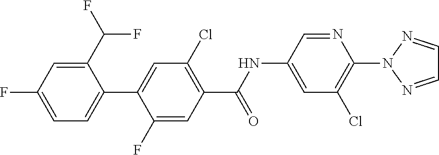 [SMILES: O=C(Nc1cnc(-n2nccn2)c(Cl)c1)c1cc(F)c(-c2ccc(F)cc2C(F)F)cc1Cl] | 5-chloro-N-(5-chloro-6- (2H-1,2,3-triazol-2- yl)pyridin-3-yl)-2′- (difluoromethyl)-2,4′- difluoro-[1,1′-biphenyl]-4- carboxamide | 496 |
| Example 205 (A205) | 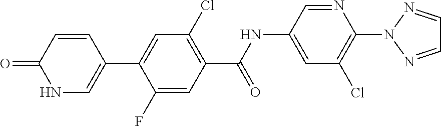 [SMILES: O=C(Nc1cnc(-n2nccn2)c(Cl)c1)c1cc(F)c(-c2ccc(=O)[nH]c2)cc1Cl] | 2-chloro-N-(5-chloro-6- (2H-1,2,3-triazol-2- yl)pyridin-3-yl)-5-fluoro-4- (6-oxo-1,6-dihydropyridin- 3-yl)benzamide | 445 |
| Example 206 (A206) | 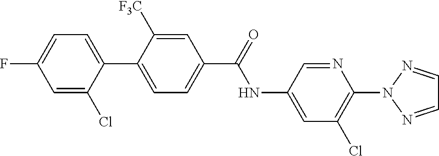 [SMILES: O=C(Nc1cnc(-n2nccn2)c(Cl)c1)c1ccc(-c2ccc(F)cc2Cl)c(C(F)(F)F)c1] | N-(5-chloro-6-(2H-1,2,3- triazol-2-yl)pyridin-3-yl)- 2,4′-difluoro-2′-hydroxy-5- methyl-[1,1′-biphenyl]-4- carboxamide | 496 |
| Example 207 (A207) | 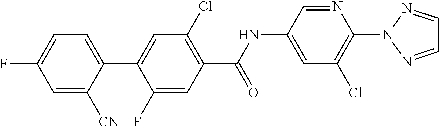 [SMILES: N#Cc1cc(F)ccc1-c1cc(Cl)c(C(=O)Nc2cnc(-n3nccn3)c(Cl)c2)cc1F] | 5-chloro-N-(5-chloro-6- (2H-1,2,3-triazol-2- yl)pyridin-3-yl)-2′-cyano- 2,4′-difluoro-[1,1′- biphenyl]-4-carboxamide | 472 |
| Example 208 (A208) | 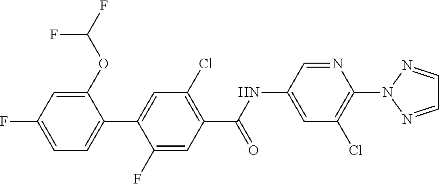 [SMILES: O=C(Nc1cnc(-n2nccn2)c(Cl)c1)c1cc(F)c(-c2ccc(F)cc2OC(F)F)cc1Cl] | 5-chloro-N-(5-chloro-6- (2H-1,2,3-triazol-2- yl)pyridin-3-yl)-2′- (difluoromethoxy)-2,4′- difluoro-[1,1′-biphenyl]-4- carboxamide | 512 |
| Example 209 (A209) | 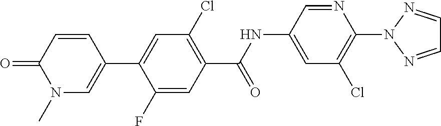 [SMILES: Cn1cc(-c2cc(Cl)c(C(=O)Nc3cnc(-n4nccn4)c(Cl)c3)cc2F)ccc1=O] | 2-chloro-N-(5-chloro-6- (2H-1,2,3-triazol-2- yl)pyridin-3-yl)-5-fluoro-4- (1-methyl-6-oxo-1,6- dihydropyridin-3- yl)benzamide | 459 |
| Example 210 (A210) | 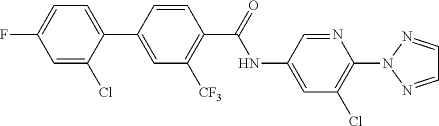 [SMILES: O=C(Nc1cnc(-n2nccn2)c(Cl)c1)c1ccc(-c2ccc(F)cc2Cl)cc1C(F)(F)F] | 2′-chloro-N-(5-chloro-6- (2H-1,2,3-triazol-2- yl)pyridin-3-yl)-4′-fluoro- 3-( trifluoromethyl)-[1,1′- biphenyl]-4- carboxamide | 496 |
| Example 211 (A211) | 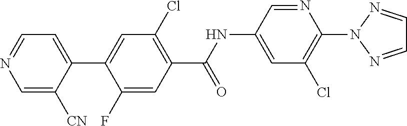 [SMILES: N#Cc1cnccc1-c1cc(Cl)c(C(=O)Nc2cnc(-n3nccn3)c(Cl)c2)cc1F] | 2-chloro-N-(5-chloro-6- (2H-1,2,3-triazol-2- yl)pyridin-3-yl)-4-(3- cyanopyridin-4-yl)-5- fluorobenzamide | 454 |
| Example 212 (A212) | 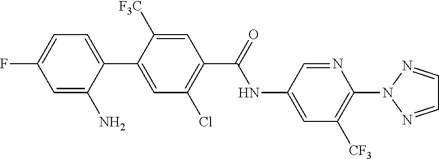 [SMILES: Nc1cc(F)ccc1-c1cc(Cl)c(C(=O)Nc2cnc(-n3nccn3)c(C(F)(F)F)c2)cc1C(F)(F)F] | N-(6-(2H-1,2,3-triazol-2- yl)-5- (trifluoromethyl)pyridin-3- yl)-2′-amino-5-chloro-4′- fluoro-2-(trifluoromethyl)- [1,1′-biphenyl]-4- carboxamide | 545 |
| Example 213 (A213) | 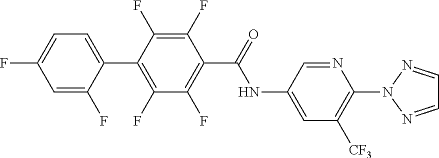 [SMILES: O=C(Nc1cnc(-n2nccn2)c(C(F)(F)F)c1)c1c(F)c(F)c(-c2ccc(F)cc2F)c(F)c1F] | N-(6-(2H-1,2,3-triazol-2- yl)-5- (trifluoromethyl)pyridin-3- yl)-2,2′,3,4′,5,6-hexafluoro- [1,1′-biphenyl]-4- carboxamide | 518 |
| Example 214 (A214) | 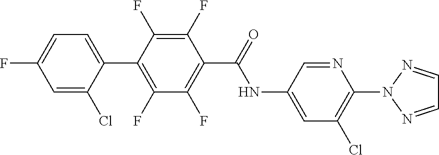 [SMILES: O=C(Nc1cnc(-n2nccn2)c(Cl)c1)c1c(F)c(F)c(-c2ccc(F)cc2Cl)c(F)c1F] | 2′-chloro-N-(5-chloro-6- (2H-1,2,3-triazol-2- yl)pyridin-3-yl)-2,3,4′,5,6- pentafluoro-[1,1′-biphenyl]- 4-carboxamide | 500 |
| Example 215 (A215) | 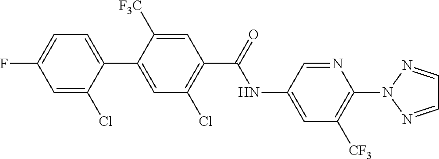 [SMILES: O=C(Nc1cnc(-n2nccn2)c(C(F)(F)F)c1)c1cc(C(F)(F)F)c(-c2ccc(F)cc2Cl)cc1Cl] | N-(6-(2H-1,2,3-triazol-2- yl)-5- (trifluoromethyl)pyridin-3- yl)-2′,5-dichloro-4′-fluoro- 2-(trifluoromethyl)-[1,1′- biphenyl]-4-carboxamide | 564 |
| Example 216 (A216) | 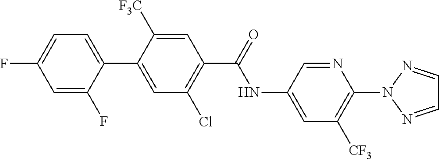 [SMILES: O=C(Nc1cnc(-n2nccn2)c(C(F)(F)F)c1)c1cc(C(F)(F)F)c(-c2ccc(F)cc2F)cc1Cl] | N-(6-(2H-1,2,3-triazol-2- yl)-5- (trifluoromethyl)pyridin-3- yl)-5-chloro-2′,4′-difluoro- 2-(trifluoromethyl)-[1,1′- biphenyl]-4-carboxamide | 548 |
| Example 217 (A217) | 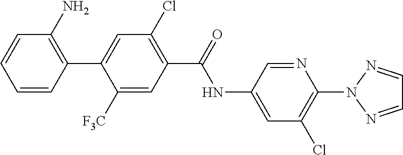 [SMILES: Nc1ccccc1-c1cc(Cl)c(C(=O)Nc2cnc(-n3nccn3)c(Cl)c2)cc1C(F)(F)F] | 2′-amino-5-chloro-N-(5- chloro-6-(2H-1,2,3-triazol- 2-yl)pyridin-3-yl)-2- (trifluoromethyl)-[1,1′- biphenyl]-4-carboxamide | 493 |
| Example 218 (A218) | 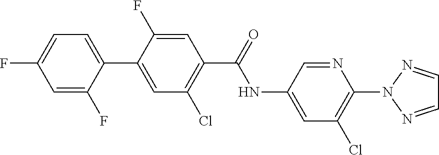 [SMILES: O=C(Nc1cnc(-n2nccn2)c(Cl)c1)c1cc(F)c(-c2ccc(F)cc2F)cc1Cl] | 5-chloro-N-(5-chloro-6- (2H-1,2,3-triazol-2- yl)pyridin-3-yl)-2,2′,4′- trifluoro-[1,1′-biphenyl]-4- carboxamide | 464 |
| Example 219 (A219) | 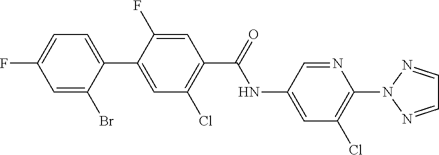 [SMILES: O=C(Nc1cnc(-n2nccn2)c(Cl)c1)c1cc(F)c(-c2ccc(F)cc2Br)cc1Cl] | 2′-bromo-5-chloro-N-(5- chloro-6-(2H-1,2,3-triazol- 2-yl)pyridin-3-yl)-2,4′- difluoro-[1,1′-biphenyl]-4- carboxamide | 524 |
| Example 221 (A221) | 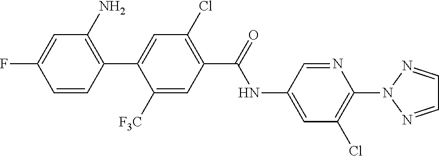 [SMILES: Nc1cc(F)ccc1-c1cc(Cl)c(C(=O)Nc2cnc(-n3nccn3)c(Cl)c2)cc1C(F)(F)F] | 2′-amino-5-chloro-N-(5- chloro-6-(2H-1,2,3-triazol- 2-yl)pyridin-3-yl)-2- cyclopropyl-4′-fluoro-[1,1′- biphenyl]-4-carboxamide | 483 |
| Example 222 (A222) | 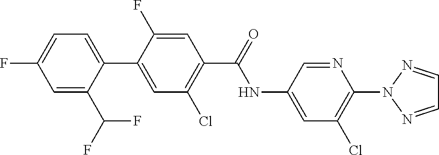 [SMILES: O=C(Nc1cnc(-n2nccn2)c(Cl)c1)c1cc(F)c(-c2ccc(F)cc2C(F)F)cc1Cl] | 5-chloro-N-(5-chloro-6- (2H-1,2,3-triazol-2- yl)pyridin-3-yl)-2′- (difluoromethyl)-2,4′- difluoro-[1,1′-biphenyl]-4- carboxamide | 496 |
| Example 223 (A223) | 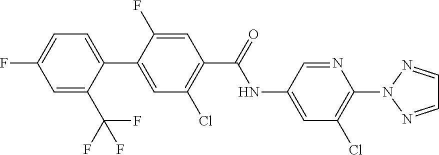 [SMILES: O=C(Nc1cnc(-n2nccn2)c(Cl)c1)c1cc(F)c(-c2ccc(F)cc2C(F)(F)F)cc1Cl] | 5-chloro-N-(5-chloro-6- (2H-1,2,3-triazol-2- yl)pyridin-3-yl)-2,4′- difluoro-2′- (trifluoromethyl)-[1,1′- biphenyl]-4-carboxamide | 514 |
| Example 224 (A224) | 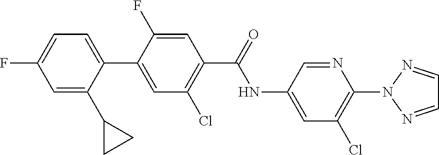 [SMILES: O=C(Nc1cnc(-n2nccn2)c(Cl)c1)c1cc(F)c(-c2ccc(F)cc2C2CC2)cc1Cl] | 5-chloro-N-(5-chloro-6- (2H-1,2,3-triazol-2- yl)pyridin-3-yl)-2′- cyclopropyl-2,4′-difluoro- [1,1′-biphenyl]-4- carboxamide | 486 |
| Example 225 (A225) | 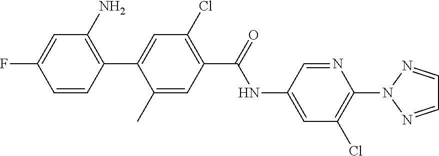 [SMILES: Cc1cc(C(=O)Nc2cnc(-n3nccn3)c(Cl)c2)c(Cl)cc1-c1ccc(F)cc1N] | 2′-amino-5-chloro-N-(5- chloro-6-(2H-1,2,3-triazol- 2-yl)pyridin-3-yl)-4′- fluoro-2-methyl-[1,1′- biphenyl]-4-carboxamide | 457 |
| Example 226 (A226) | 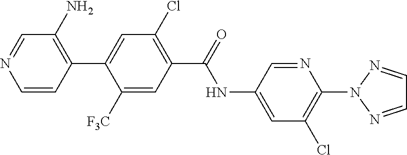 [SMILES: Nc1cnccc1-c1cc(Cl)c(C(=O)Nc2cnc(-n3nccn3)c(Cl)c2)cc1C(F)(F)F] | 4-(3-aminopyridin-4-yl)-2- chloro-N-(5-chloro-6-(2H- 1,2,3-triazol-2-yl)pyridin- 3-yl)-5- (trifluoromethyl)benzamide | 494 |
| Example 227 (A227) | 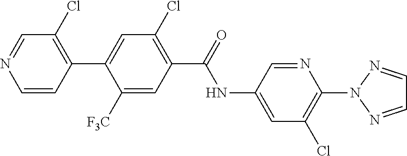 [SMILES: O=C(Nc1cnc(-n2nccn2)c(Cl)c1)c1cc(C(F)(F)F)c(-c2ccncc2Cl)cc1Cl] | 2-chloro-N-(5-chloro-6- (2H-1,2,3-triazol-2- yl)pyridin-3-yl)-4-(3- chloropyridin-4-yl)-5- (trifluoromethyl)benzamide | 513 |
| Example 228 (A228) | 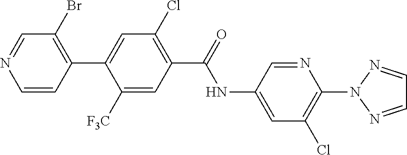 [SMILES: O=C(Nc1cnc(-n2nccn2)c(Cl)c1)c1cc(C(F)(F)F)c(-c2ccncc2Br)cc1Cl] | 4-(3-bromopyridin-4-yl)-2- chloro-N-(5-chloro-6-(2H- 1,2,3-triazol-2-yl)pyridin- 3-yl)-5- (trifluoromethyl)benzamide | 557 |
| Example 229 (A229) | 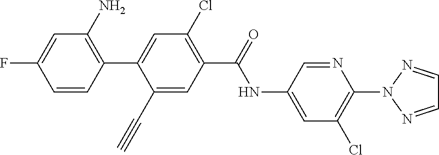 [SMILES: C#Cc1cc(C(=O)Nc2cnc(-n3nccn3)c(Cl)c2)c(Cl)cc1-c1ccc(F)cc1N] | 2′-amino-5-chloro-N-(5- chloro-6-(2H-1,2,3-triazol- 2-yl)pyridin-3-yl)-2- ethynyl-4′-fluoro-[1,1′- biphenyl]-4-carboxamide | 467 |
| Example 230 (A230) | 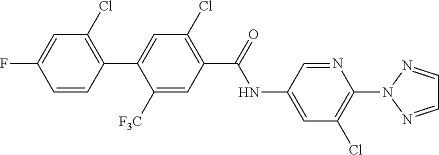 [SMILES: O=C(Nc1cnc(-n2nccn2)c(Cl)c1)c1cc(C(F)(F)F)c(-c2ccc(F)cc2Cl)cc1Cl] | 2′,5-dichloro-N-(5-chloro- 6-(2H-1,2,3-triazol-2- yl)pyridin-3-yl)-4′-fluoro- 2-(trifluoromethyl)-[1,1′- biphenyl]-4-carboxamide | 530 |
| Example 231 (A231) | 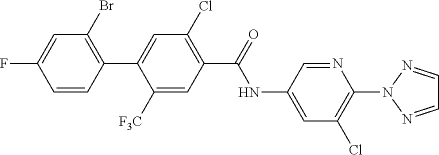 [SMILES: O=C(Nc1cnc(-n2nccn2)c(Cl)c1)c1cc(C(F)(F)F)c(-c2ccc(F)cc2Br)cc1Cl] | 2′-bromo-5-chloro-N-(5- chloro-6-(2H-1,2,3-triazol- 2-yl)pyridin-3-yl)-4′- fluoro-2-(trifluoromethyl)- [1,1′-biphenyl]-4- carboxamide | 574 |
| Example 232 (A232) | 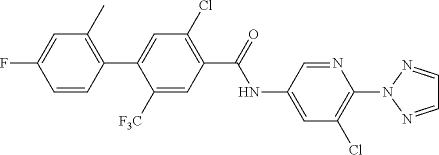 [SMILES: Cc1cc(F)ccc1-c1cc(Cl)c(C(=O)Nc2cnc(-n3nccn3)c(Cl)c2)cc1C(F)(F)F] | 5-chloro-N-(5-chloro-6- (2H-1,2,3-triazol-2- yl)pyridin-3-yl)-4′-fluoro- 2′-methyl-2- (trifluoromethyl)-[1,1′- biphenyl]-4-carboxamide | 510 |
| Example 233 (A233) | 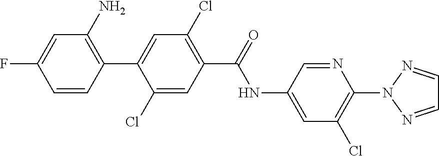 [SMILES: Nc1cc(F)ccc1-c1cc(Cl)c(C(=O)Nc2cnc(-n3nccn3)c(Cl)c2)cc1Cl] | 2′-amino-2,5-dichloro-N- (5-chloro-6-(2H-1,2,3- triazol-2-yl)pyridin-3-yl)- 4′-fluoro-[1,1′-biphenyl]-4- carboxamide | 477 |
| Example 234 (A234) | 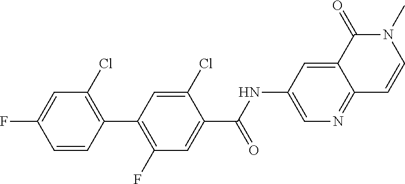 [SMILES: Cn1ccc2ncc(NC(=O)c3cc(F)c(-c4ccc(F)cc4Cl)cc3Cl)cc2c1=O] | 2′,5-dichloro-2,4′-difluoro- N-(6-methyl-5-oxo-5,6- dihydro-1,6-naphthyridin- 3-yl)-[1,1′-biphenyl]-4- carboxamide | 460 |
| Example 235 (A235) | 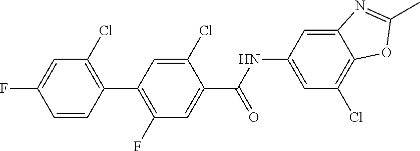 [SMILES: Cc1nc2cc(NC(=O)c3cc(F)c(-c4ccc(F)cc4Cl)cc3Cl)cc(Cl)c2o1] | 2′,5-dichloro-N-(7-chloro- 2-methylbenzo[d]oxazol-5- yl)-2,4′-difluoro-[1,1′- biphenyl]-4-carboxamide | 467 |
| Example 236 (A236) | 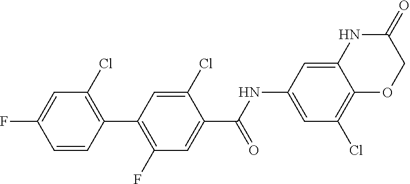 [SMILES: O=C1COc2c(Cl)cc(NC(=O)c3cc(F)c(-c4ccc(F)cc4Cl)cc3Cl)cc2N1] | 2′,5-dichloro-N-(8-chloro- 3-oxo-3,4-dihydro-2H- benzo[b][1,4]oxazin-6-yl)- 2,4′-difluoro-[1,1′- biphenyl]-4-carboxamide | 483 |
| Example 237 (A237) | 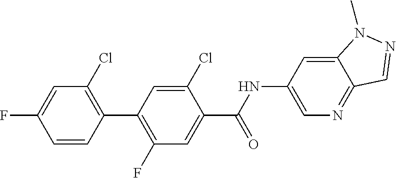 [SMILES: Cn1ncc2ncc(NC(=O)c3cc(F)c(-c4ccc(F)cc4Cl)cc3Cl)cc21] | 2′,5-dichloro-2,4′-difluoro- N-(1-methyl-1H- pyrazolo[4,3-b]pyridin-6- yl)-[1,1′-biphenyl]-4- carboxamide | 433 |
| Example 238 (A238) | 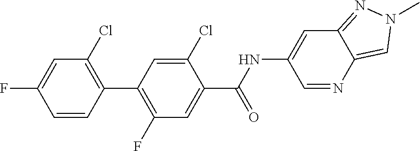 [SMILES: Cn1cc2ncc(NC(=O)c3cc(F)c(-c4ccc(F)cc4Cl)cc3Cl)cc2n1] | 2′,5-dichloro-2,4′-difluoro- N-(2-methyl-2H- pyrazolo[4,3-b]pyridin-6- yl)-[1,1′-biphenyl]-4- carboxamide | 433 |
| Example 239 (A239) | 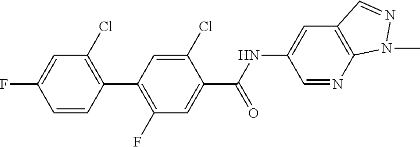 [SMILES: Cn1ncc2cc(NC(=O)c3cc(F)c(-c4ccc(F)cc4Cl)cc3Cl)cnc21] | 2′,5-dichloro-2,4′-difluoro- N-(1-methyl-1H- pyrazolo[3,4-b]pyridin-5- yl)-[1,1′-biphenyl]-4- carboxamide | 433 |
| Example 240 (A240) | 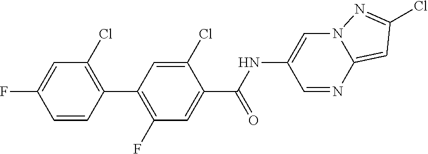 [SMILES: O=C(Nc1cnc2cc(Cl)nn2c1)c1cc(F)c(-c2ccc(F)cc2Cl)cc1Cl] | 2′,5-dichloro-N-(2- chloropyrazolo[1,5- alpyrimidin-6-yl)-2,4′- difluoro-[1,1′-biphenyl]-4- carboxamide | 453 |
| Example 241 (A241) | 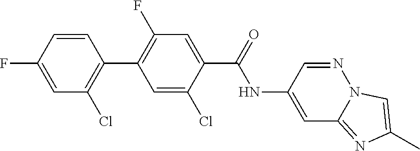 [SMILES: Cc1cn2ncc(NC(=O)c3cc(F)c(-c4ccc(F)cc4Cl)cc3Cl)cc2n1] | 2′,5-dichloro-2,4′-difluoro- N-(2-methylimidazo[1,2- b]pyridazin-7-yl)-[1,1′- biphenyl]-4-carboxamide | 433 |
| Example 242 (A242) | 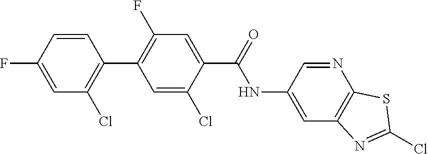 [SMILES: O=C(Nc1cnc2sc(Cl)nc2c1)c1cc(F)c(-c2ccc(F)cc2Cl)cc1Cl] | 2′,5-dichloro-N-(2- chlorothiazolo[5,4- b]pyridin-6-yl)-2,4′- difluoro-[1,1′-biphenyl]-4- carboxamide | 470 |
| Example 243 (A243) | 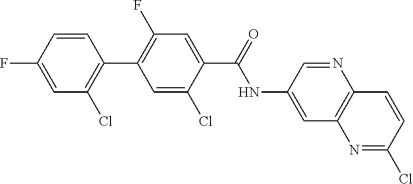 [SMILES: O=C(Nc1cnc2ccc(Cl)nc2c1)c1cc(F)c(-c2ccc(F)cc2Cl)cc1Cl] | 2′,5-dichloro-N-(6-chloro- 1,5-naphthyridin-3-yl)-2,4′- difluoro-[1,1′-biphenyl]-4- carboxamide | 464 |
| Example 244 (A244) | 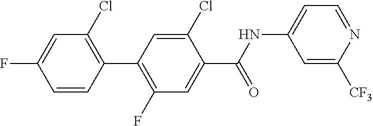 [SMILES: O=C(Nc1ccnc(C(F)(F)F)c1)c1cc(F)c(-c2ccc(F)cc2Cl)cc1Cl] | 2′,5-dichloro-2,4′-difluoro- N-(2- (trifluoromethyl)pyridin-4- yl)-[1,1′-biphenyl]-4- carboxamide | 447 |
| Example 245 (A245) | 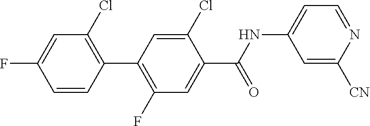 [SMILES: N#Cc1cc(NC(=O)c2cc(F)c(-c3ccc(F)cc3Cl)cc2Cl)ccn1] | 2′,5-dichloro-N-(2- cyanopyridin-4-yl)-2,4′- difluoro-[1,1′-biphenyl]-4- carboxamide | 404 |
| Example 246 (A246) | 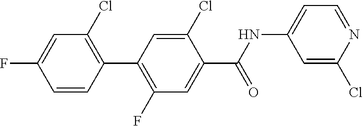 [SMILES: O=C(Nc1ccnc(Cl)c1)c1cc(F)c(-c2ccc(F)cc2Cl)cc1Cl] | 2′,5-dichloro-N-(2- chloropyridin-4-yl)-2,4′- difluoro-[1,1′-biphenyl]-4- carboxamide | 413 |
| Example 247 (A247) | 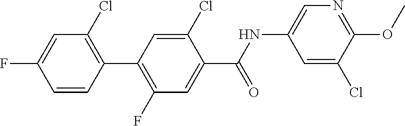 [SMILES: COc1ncc(NC(=O)c2cc(F)c(-c3ccc(F)cc3Cl)cc2Cl)cc1Cl] | 2′,5-dichloro-N-(5-chloro- 6-methoxypyridin-3-yl)- 2,4′-difluoro-[1,1′- biphenyl]-4-carboxamide | 443 |
| Example 248 (A248) | 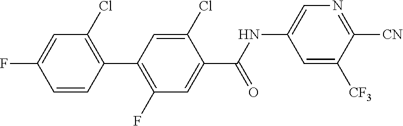 [SMILES: N#Cc1ncc(NC(=O)c2cc(F)c(-c3ccc(F)cc3Cl)cc2Cl)cc1C(F)(F)F] | 2′,5-dichloro-N-(6-cyano-5- (trifluoromethyl)pyridin-3- yl)-2,4′-difluoro-[1,1′- biphenyl]-4-carboxamide | 472 |
| Example 249 (A249) | 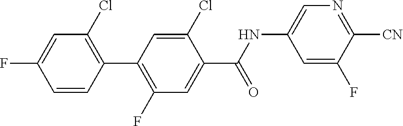 [SMILES: N#Cc1ncc(NC(=O)c2cc(F)c(-c3ccc(F)cc3Cl)cc2Cl)cc1F] | 2′,5-dichloro-N-(6-cyano-5- fluoropyridin-3-yl)-2,4′- difluoro-[1,1′-biphenyl]-4- carboxamide | 422 |
| Example 250 (A250) | 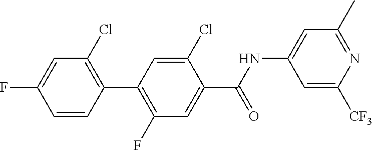 [SMILES: Cc1cc(NC(=O)c2cc(F)c(-c3ccc(F)cc3Cl)cc2Cl)cc(C(F)(F)F)n1] | 2′,5-dichloro-2,4′-difluoro- N-(2-methyl-6- (trifluoromethyl)pyridin-4- yl)-[1,1′-biphenyl]-4- carboxamide | 461 |
| Example 251 (A251) | 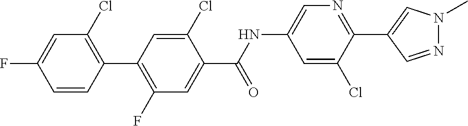 [SMILES: Cn1cc(-c2ncc(NC(=O)c3cc(F)c(-c4ccc(F)cc4Cl)cc3Cl)cc2Cl)cn1] | 2′,5-dichloro-N-(5-chloro- 6-(1-methyl-1 H-pyrazol-4- yl)pyridin-3-yl)-2,4′- difluoro-[1,1′-biphenyl]-4- carboxamide | 493 |
| Example 252 (A252) | 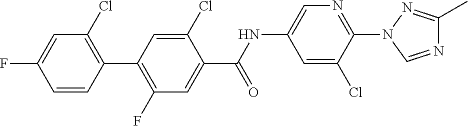 [SMILES: Cc1ncn(-c2ncc(NC(=O)c3cc(F)c(-c4ccc(F)cc4Cl)cc3Cl)cc2Cl)n1] | 2′,5-dichloro-N-(5-chloro- 6-(3-methyl-1H-1,2,4- triazol-1-yl)pyridin-3-yl)- 2,4′-difluoro-[1,1′- biphenyl]-4-carboxamide | 494 |
| Example 253 (A253) | 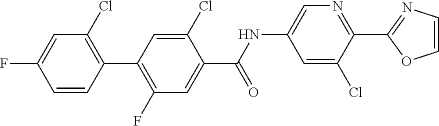 [SMILES: O=C(Nc1cnc(-c2ncco2)c(Cl)c1)c1cc(F)c(-c2ccc(F)cc2Cl)cc1Cl] | 2′,5-dichloro-N-(5-chloro- 6-(oxazol-2-yl)pyridin-3- yl)-2,4′-difluoro-[1,1′- biphenyl]-4-carboxamide | 480 |
| Example 254 (A254) | 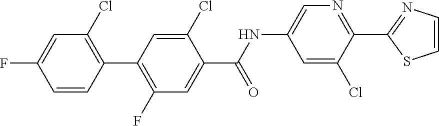 [SMILES: O=C(Nc1cnc(-c2nccs2)c(Cl)c1)c1cc(F)c(-c2ccc(F)cc2Cl)cc1Cl] | 2′,5-dichloro-N-(5-chloro- 6-(thiazol-2-yl)pyridin-3- yl)-2,4′-difluoro-[1,1′- biphenyl]-4-carboxamide | 496 |
| Example 255 (A255) | 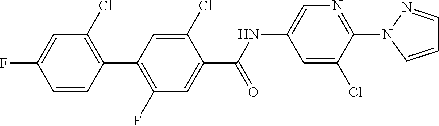 [SMILES: O=C(Nc1cnc(-n2cccn2)c(Cl)c1)c1cc(F)c(-c2ccc(F)cc2Cl)cc1Cl] | 2′,5-dichloro-N-(5-chloro- 6-(1H-pyrazol-1- yl)pyridin-3-yl)-2,4′- difluoro-[1,1′-biphenyl]-4- carboxamide | 479 |
| Example 256 (A256) | 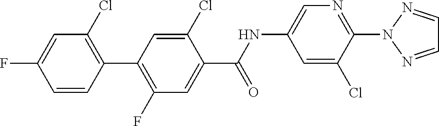 [SMILES: O=C(Nc1cnc(-n2nccn2)c(Cl)c1)c1cc(F)c(-c2ccc(F)cc2Cl)cc1Cl] | 2′,5-dichloro-N-(5-cyano-6- (2H-1,2,3-triazol-2- yl)pyridin-3-yl)-2,4′- difluoro-[1,1′-biphenyl]-4- carboxamide | 471 |
| Example 257 (A257) | 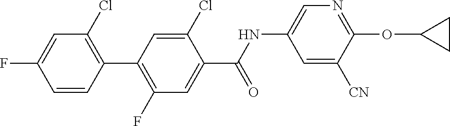 [SMILES: N#Cc1cc(NC(=O)c2cc(F)c(-c3ccc(F)cc3Cl)cc2Cl)cnc1OC1CC1] | 2′,5-dichloro-N-(5-cyano-6- cyclopropoxypyridin-3-yl)- 2,4′-difluoro-[1,1′- biphenyl]-4-carboxamide | 460 |
| Example 258 (A258) | 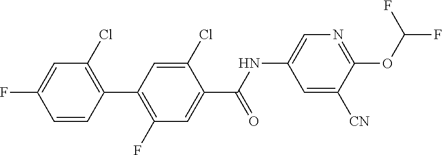 [SMILES: N#Cc1cc(NC(=O)c2cc(F)c(-c3ccc(F)cc3Cl)cc2Cl)cnc1OC(F)F] | 2′,5-dichloro-N-(5-cyano-6- (difluoromethoxy)pyridin- 3-yl)-2,4′-difluoro-[1,1′- biphenyl]-4-carboxamide | 470 |
| Example 259 (A259) | 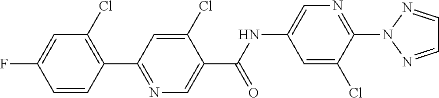 [SMILES: O=C(Nc1cnc(-n2nccn2)c(Cl)c1)c1cnc(-c2ccc(F)cc2Cl)cc1Cl] | 4-chloro-6-(2-chloro-4- fluorophenyl)-N-(5-chloro- 6-(2H-1,2,3-triazol-2- yl)pyridin-3- yl)nicotinamide | 463 |
| Example 260 (A260) | 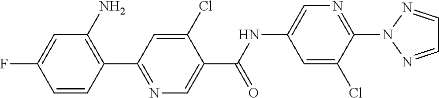 [SMILES: Nc1cc(F)ccc1-c1cc(Cl)c(C(=O)Nc2cnc(-n3nccn3)c(Cl)c2)cn1] | 6-(2-amino-4- fluorophenyl)-4-chloro-N- (5-chloro-6-(2H-1,2,3- triazol-2-yl)pyridin-3- yl)nicotinamide | 444 |
| Example 261 (A261) | 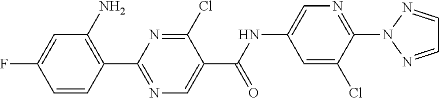 [SMILES: Nc1cc(F)ccc1-c1ncc(C(=O)Nc2cnc(-n3nccn3)c(Cl)c2)c(Cl)n1] | 2-(2-amino-4- fluorophenyl)-4-chloro-N- (5-chloro-6-(2H-1,2,3- triazol-2-yl)pyridin-3- yl)pyrimidine-5- carboxamide | 445 |
| Example 262 (A262) | 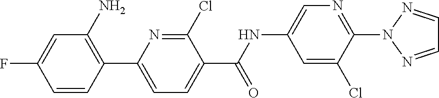 [SMILES: Nc1cc(F)ccc1-c1ccc(C(=O)Nc2cnc(-n3nccn3)c(Cl)c2)c(Cl)n1] | 6-(2-amino-4- fluorophenyl)-2-chloro-N- (5-chloro-6-(2H-1,2,3- triazol-2-yl)pyridin-3- yl)nicotinamide | 444 |
| Example 263 (A263) | 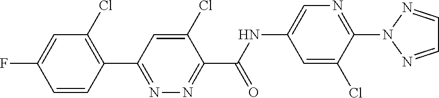 [SMILES: O=C(Nc1cnc(-n2nccn2)c(Cl)c1)c1nnc(-c2ccc(F)cc2Cl)cc1Cl] | 4-chloro-6-(2-chloro-4- fluorophenyl)-N-(5-chloro- 6-(2H-1,2,3-triazol-2- yl)pyridin-3-yl)pyridazine- 3-carboxamide | 464 |
| Example 264 (A264) | 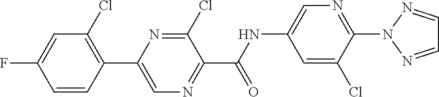 [SMILES: O=C(Nc1cnc(-n2nccn2)c(Cl)c1)c1ncc(-c2ccc(F)cc2Cl)nc1Cl] | 3-chloro-5-(2-chloro-4- fluorophenyl)-N-(5-chloro- 6-(2H-1,2,3-triazol-2- yl)pyridin-3-yl)pyrazine-2- carboxamide | 464 |
| Example 265 (A265) | 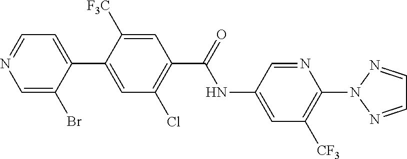 [SMILES: O=C(Nc1cnc(-n2nccn2)c(C(F)(F)F)c1)c1cc(C(F)(F)F)c(-c2ccncc2Br)cc1Cl] | N-(6-(2H-1,2,3-triazol-2- yl)-5- (trifluoromethyl)pyridin-3- yl)-4-(3-bromopyridin-4- yl)-2-chloro-5- (trifluoromethyl)benzamide | 591 |
| Example 266 (A266) | 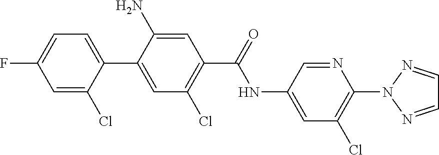 [SMILES: Nc1cc(C(=O)Nc2cnc(-n3nccn3)c(Cl)c2)c(Cl)cc1-c1ccc(F)cc1Cl] | 2-amino-2′,5-dichloro-N- (5-chloro-6-(2H-1,2,3- triazol-2-yl)pyridin-3-yl)- 4′-fluoro-[1,1′-biphenyl]-4- carboxamide | 477 |
| Example 267 (A267) | 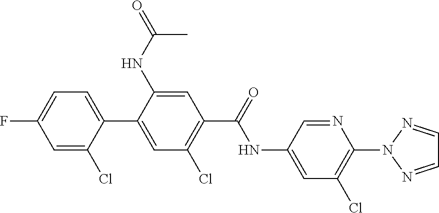 [SMILES: CC(=O)Nc1cc(C(=O)Nc2cnc(-n3nccn3)c(Cl)c2)c(Cl)cc1-c1ccc(F)cc1Cl] | 2-acetamido-2′,5-dichloro- N-(5-chloro-6-(2H-1,2,3- triazol-2-yl)pyridin-3-yl)- 4′-fluoro-[1,1′-biphenyl]-4- carboxamide | 519 |
| Example 268 (A268) | 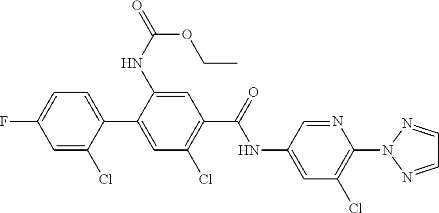 [SMILES: CCOC(=O)Nc1cc(C(=O)Nc2cnc(-n3nccn3)c(Cl)c2)c(Cl)cc1-c1ccc(F)cc1Cl] | ethyl(2′,5-dichloro-4-((5- chloro-6-(2H-1,2,3-triazol- 2-yl)pyridin-3- yl)aminoformyl)-4′-fluoro- [1,1′-biphenyl]-2- yl)aminoformate | 549 |
| Example 269 (A269) | 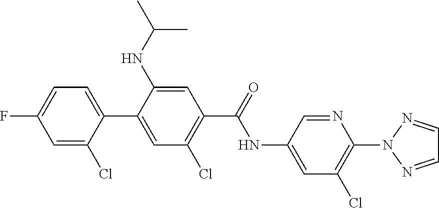 [SMILES: CC(C)Nc1cc(C(=O)Nc2cnc(-n3nccn3)c(Cl)c2)c(Cl)cc1-c1ccc(F)cc1Cl] | 2′,5-dichloro-N-(5-chloro- 6-(2H-1,2,3-triazol-2- yl)pyridin-3-yl)-4′-fluoro- 2-(isopropylamino)-[1,1′- biphenyl]-4-carboxamide | 519 |
| Example 270 (A270) | 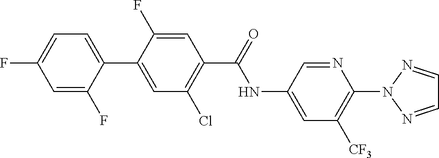 [SMILES: O=C(Nc1cnc(-n2nccn2)c(C(F)(F)F)c1)c1cc(F)c(-c2ccc(F)cc2F)cc1Cl] | N-(6-(2H-1,2,3-triazol-2- yl)-5- (trifluoromethyl)pyridin-3- yl)-5-chloro-2,2′,4′- trifluoro-[1,1′-biphenyl]-4- carboxamide | 498 |
| Example 271 (A271) | 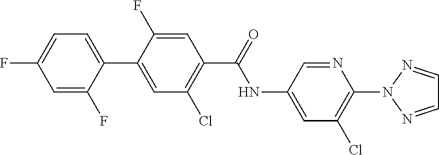 [SMILES: O=C(Nc1cnc(-n2nccn2)c(Cl)c1)c1cc(F)c(-c2ccc(F)cc2F)cc1Cl] | 5-chloro-N-(5-chloro-6- (2H-1,2,3-triazol-2- yl)pyridin-3-yl)-2,2′,4′- trifluoro-[1,1′-biphenyl]-4- carboxamide | 464 |
| Example 272 (A272) | 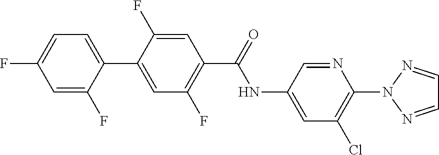 [SMILES: O=C(Nc1cnc(-n2nccn2)c(Cl)c1)c1cc(F)c(-c2ccc(F)cc2F)cc1F] | N-(5-chloro-6-(2H-1,2,3- triazol-2-yl)pyridin-3-yl)- 2,2′,4′,5-tetrafluoro-[1,1′- biphenyl]-4-carboxamide | 448 |
| Example 273 (A273) | 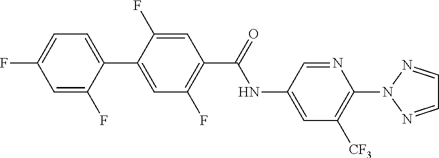 [SMILES: O=C(Nc1cnc(-n2nccn2)c(C(F)(F)F)c1)c1cc(F)c(-c2ccc(F)cc2F)cc1F] | N-(6-(2H-1,2,3-triazol-2- yl)-5- (trifluoromethyl)pyridin-3- yl)-2,2′,4′,5-tetrafluoro- [1,1′-biphenyl]-4- carboxamide | 482 |
| Example 275 (A275) | 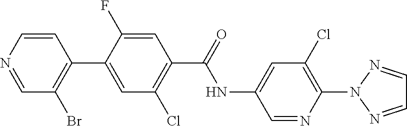 [SMILES: O=C(Nc1cnc(-n2nccn2)c(Cl)c1)c1cc(F)c(-c2ccncc2Br)cc1Cl] | 4-(3-bromopyridin-4-yl)-2- chloro-N-(5-chloro-6-(2H- 1,2,3-triazol-2-yl)pyridin- 3-yl)-5-fluorobenzamide | 506 |

### A121:

¹H NMR (500 MHz, DMSO-d₆) δ 11.44 (s, 1H), 8.85 (d, J=2.3 Hz, 1H), 8.67 (d, J=2.3 Hz, 1H), 8.19 (s, 2H), 7.77 (d, J=9.2 Hz, 1H), 7.60 (d, J=6.4 Hz, 1H), 7.05 (dd, J=8.4, 6.6 Hz, 1H), 6.66 (dd, J=11.5, 2.7 Hz, 1H), 6.59-6.44 (in, 1H).

### A122:

¹H NMR (500 MHz, DMSO-d₆) δ 11.50 (s, 1H), 9.12 (d, J=2.4 Hz, 1H), 8.90 (d, J=2.4 Hz, 1H), 8.21 (s, 2H), 7.78 (d, J=9.2 Hz, 1H), 7.59 (d, J=6.5 Hz, 1H), 7.00 (dd, J=8.5, 6.7 Hz, 1H), 6.56 (dd, J=11.7, 2.6 Hz, 1H), 6.47-6.34 (m, 1H).

### A123:

¹H NMR (500 MHz, DMSO-d₆) δ 11.34 (s, 1H), 9.17 (d, J=2.4 Hz, 1H), 8.90 (d, J=2.4 Hz, 1H), 8.21 (s, 2H), 8.00 (d, J=6.5 Hz, 1H), 7.43 (d, J=10.2 Hz, 1H), 6.93 (dd, J=8.4, 6.7 Hz, 1H), 6.54 (dd, J=11.7, 2.6 Hz, 1H), 6.42 (td, J=8.5, 2.6 Hz, 1H), 5.21 (s, 2H).

### A217:

¹H NMR (500 MHz, DMSO-d₆) δ 11.42 (s, 1H), 8.82 (d, J=2.3 Hz, 1H), 8.66 (d, J=2.3 Hz, 1H), 8.20 (d, J=7.0 Hz, 3H), 7.58 (s, 1H), 7.12 (td, J=8.3, 7.9, 1.6 Hz, 1H), 6.84 (d, J=7.5 Hz, 1H), 6.76 (dd, J=8.2, 1.1 Hz, 1H), 6.61 (td, J=7.4, 1.1 Hz, 1H), 4.78 (s, 2H).

### A116:

¹H NMR (500 MHz, DMSO-d₆) δ 11.23 (s, 1H), 8.85 (d, J=2.3 Hz, 1H), 8.68 (d, J=2.3 Hz, 1H), 8.50 (s, OH), 8.18 (s, 2H), 7.74-7.53 (m, 2H), 7.54-7.37 (m, 2H), 7.27 (td, J=8.4, 2.5 Hz, 1H), 2.45 (s, 3H).

### A275:

¹H NMR (500 MHz, DMSO-d₆) δ 11.53 (s, 1H), 8.95 (s, 1H), 8.81 (s, 1H), 8.68 (d, J=28.1 Hz, 2H), 8.19 (s, 2H), 8.03-7.67 (m, 2H), 7.58 (s, 1H).

### Example 281: Synthesis of Compound A281

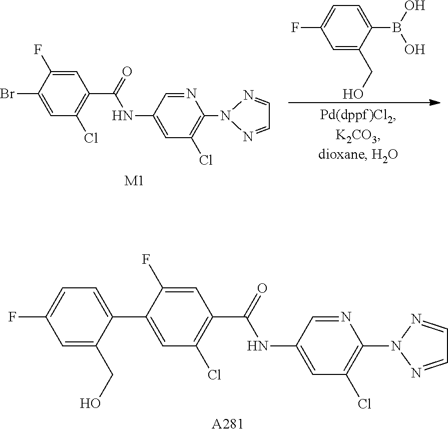 [SMILES: O=C(Nc1cnc(-n2nccn2)c(Cl)c1)c1cc(F)c(Br)cc1Cl ; OCc1cc(F)ccc1B(O)O ; "M1" ; O=C(Nc1cnc(-n2nccn2)c(Cl)c1)c1cc(F)c(-c2ccc(F)cc2CO)cc1Cl ; "A281"]

Into a 50-mL round-bottom flask, was placed 4-bromo-2-chloro-N-(5-chloro-6-(2H-1,2,3-triazol-2-yl)pyridin-3-yl)-5-fluorobenzamide (M1) (43 mg), (4-Fluoro-2-(hydroxymethyl)phenyl)boronic acid (25 mg), Pd(dppf)Cl₂ (3 mg), K₂CO₃ (27 mg), dioxane (1 mL) and H₂O (0.2 mL). The resulting mixture was stirred at 90° C. under nitrogen for 8 hours. The reaction was diluted with EA (5 mL), washed with brine. The organic layer was concentrated and purified by flash column chromatography (DCM/MeOH=40/1) to give compound A281 (18 mg) as white solid. LC-MS (ES⁺): m/z 476[M+H]⁺

### Example 282: Synthesis of Compound A282

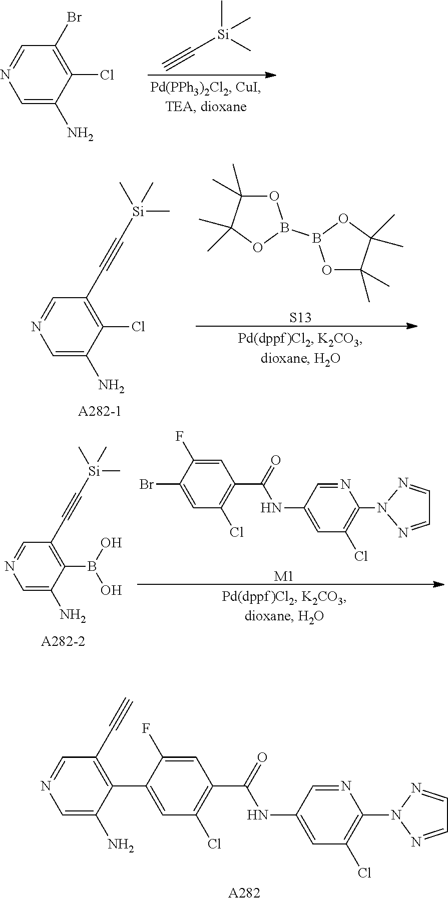

### Step 1: Synthesis of Compound A282-1

Into a 50-mL round-bottom flask, was placed 5-bromo-4-chloropyridin-3-amine (413 mg), ethynyltrimethylsilane (300 mg), Pd(PPh₃)₂Cl₂ (3 mg), CuI (5 mg), TEA (1 mL) and dioxane (10 mL). The reaction mixture was stirred at 90° C. under nitrogen for 8 hours. The mixture was diluted with EA (100 mL), washed with brine. The organic phase was concentrated and purified by flash column chromatography (DCM/MeOH=40/1) over silica gel to afford compound A282-1 (216 mg) as colorless oil. LC-MS (ES⁺): m/z 225[M+H]⁺

### Step 2: Synthesis of Compound A282-2

The mixture of A282-1 (216 mg), 4,4,4′,4′,5,5,5′,5′-octamethyl-2,2′-bis(1,3,2-dioxaborolane alkane) (500 mg), Pd(dppf)Cl₂ (30 mg), K₂CO₃ (270 mg), dioxane (10 mL) and H₂O (2 mL) was stirred at 100° C. under nitrogen for 8 hours in a 50-mL round-bottom flask. Removing of the solvent and purifying by column chromatography (DCM/MeOH=40/1) to afford compound A282-2 (88 mg) as a red oil. LC-MS (ES⁺): m/z 235[M+H]⁺

### Step 3: Synthesis of Compound A282

A mixture of 4-bromo-2-chloro-N-(5-chloro-6-(2H-1,2,3-triazol-2-yl)pyridin-3-yl)-5-fluorobenzamide (M1) (43 mg), A282-2 (38 mg), Pd(dppf)Cl₂ (3 mg), K₂CO₃ (27 mg), dioxane (1 mL) and H₂O (0.2 mL) was stirred at 90° C. for 8 hours under nitrogen. The mixture was cooled to room temperature, diluted with EA (5 mL), and washed with brine. The organic phase was concentrated and purified by flash column chromatography (DCM/MeOH=40/1) to give compound A282 (12 mg) as yellow solid. LC-MS (ES⁺): m/z 468[M+H]⁺

### Example 286: Synthesis of Compound A286

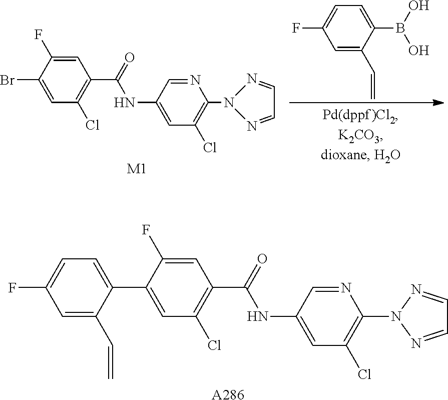 [SMILES: O=C(Nc1cnc(-n2nccn2)c(Cl)c1)c1cc(F)c(Br)cc1Cl ; C=Cc1cc(F)ccc1B(O)O ; "M1" ; C=Cc1cc(F)ccc1-c1cc(Cl)c(C(=O)Nc2cnc(-n3nccn3)c(Cl)c2)cc1F ; "A286"]

The mixture of 4-bromo-2-chloro-N-(5-chloro-6-(2H-1,2,3-triazol-2-yl)pyridin-3-yl)-5-fluorobenzamide (M1) (43 mg), (4-fluoro-2-vinylphenyl)boronic acid (24 mg), Pd(dppf)Cl₂ (3 mg), K₂CO₃ (27 mg), dioxane (1 mL) and H₂O (0.2 mL) was stirred at 90° C. for 8 hours under nitrogen. The mixture was cooled to room temperature, diluted with EA (5 mL), and washed with brine. The organic phase was concentrated and purified by flash column chromatography (DCM/MeOH=40/1) to give compound A286 (21 mg) as white solid. LC-MS (ES⁺): m/z 472[M+H]⁺

### Example 296: Synthesis of Compound A296

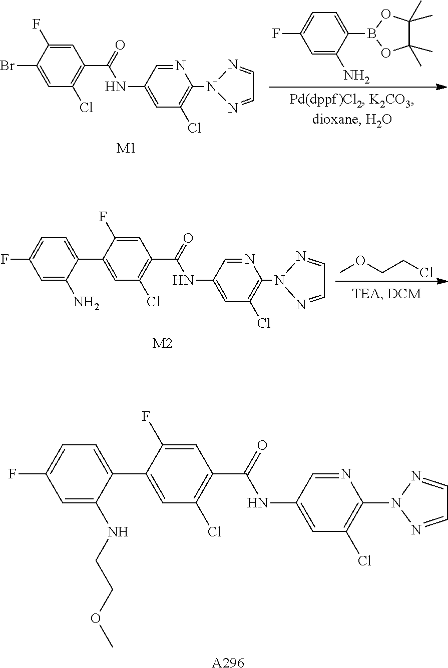

### Step 1: Synthesis of Compound M2

The mixture of 4-bromo-2-chloro-N-(5-chloro-6-(2H-1,2,3-triazol-2-yl)pyridin 3-yl)-5-fluorobenzamide (M1) (43 mg), 5-fluoro-2-(4,4,5,5-tetramethyl-1,3,2-dioxaborolan-2-yl) aniline (36 mg), Pd(dppfCl₂ (3 mg), K₂CO₃ (27 mg), dioxane (1 mL) and H₂O (0.2 mL) was stirred at 90° C. for 8 hours under nitrogen. The mixture was cooled to room temperature, diluted with EA (5 mL) and washed with brine. The organic phase was concentrated and purified by flash column chromatography (DCM/MeOH=40/1) to give compound M2 (33 mg) as white solid. LC-MS (ES⁺): m/z 461[M+H]⁺

### Step 2: Synthesis of Compound A296

To a solution of M2 (23 mg), in DCM (1 mL) was added TEA (5 mg) and 1-chloro-2-methoxyethane (6 mg). The reaction mixture was stirred at room temperature for 2 hours and diluted with EA (5 mL), washed with brine. The organic phase was concentrated and purified by flash column chromatography (DCM/MeOH=40/1) to give compound A296 (13 mg) as white solid. LC-MS (ES⁺): m/z 519 [M+H]⁺

### Example 298: Synthesis of Compound A298

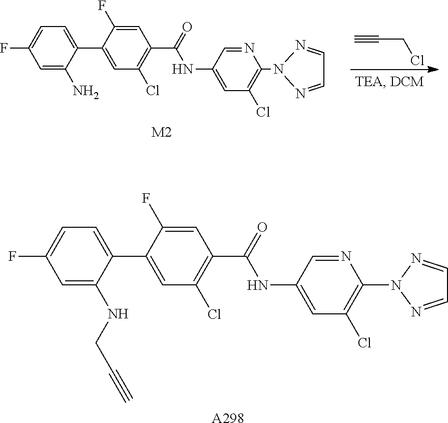

To a solution of M2 (23 mg) in DCM (mL) was added TEA (5 mg) and 3-Chloropropyl-1-yne (5 mg). The reaction mixture was stirred at room temperature for 2 hours and diluted with EA (5 mL), washed with brine. The organic phase was concentrated and purified by flash column chromatography (DCM/MeOH=40/1) to give compound A298 (18 mg), as yellow solid. LC-MS (ES⁺): m/z 499 [M+H]⁺

### Example 300: Synthesis of Compound A300

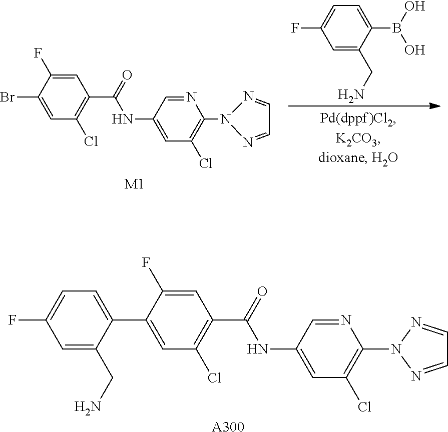 [SMILES: O=C(Nc1cnc(-n2nccn2)c(Cl)c1)c1cc(F)c(Br)cc1Cl ; NCc1cc(F)ccc1B(O)O ; "M1" ; NCc1cc(F)ccc1-c1cc(Cl)c(C(=O)Nc2cnc(-n3nccn3)c(Cl)c2)cc1F ; "A300"]

The mixture of M1 (43 mg), (2-(aminomethyl)-4-fluorophenyl)boronic acid (26 mg), Pd(dppf)Cl₂ (3 mg), K₂CO₃ (27 mg), dioxane (1 mL) and H₂O (0.2 mL) was stirred 90° C. for 8 hours under nitrogen. The mixture was cooled to room temperature, diluted with EA (5 mL) and washed with brine. The organic phase was concentrated and purified by flash column chromatography (DCM/MeOH=40/1) to give compound A300 (12 mg) as yellow solid. LC-MS (ES⁺): m/z 475[M+H]⁺

### Example 302: Synthesis of Compound A302

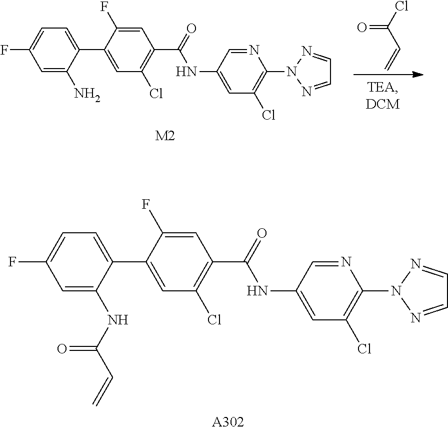

To a solution of M2 (23 mg) in DCM (1 mL) was added TEA (5 mg) and acryloyl chloride (5 mg) at 0° C. The reaction mixture was stirred at room temperature for 2 hours and diluted with EA (5 mL), washed with brine. The organic phase was concentrated and purified by flash column chromatography (DCM/MeOH=40/1) to give compound A298 (18 mg) as white solid. LC-MS (ES⁺): m/z 515[M+H]⁺

### Example 308: Synthesis of Compound A308

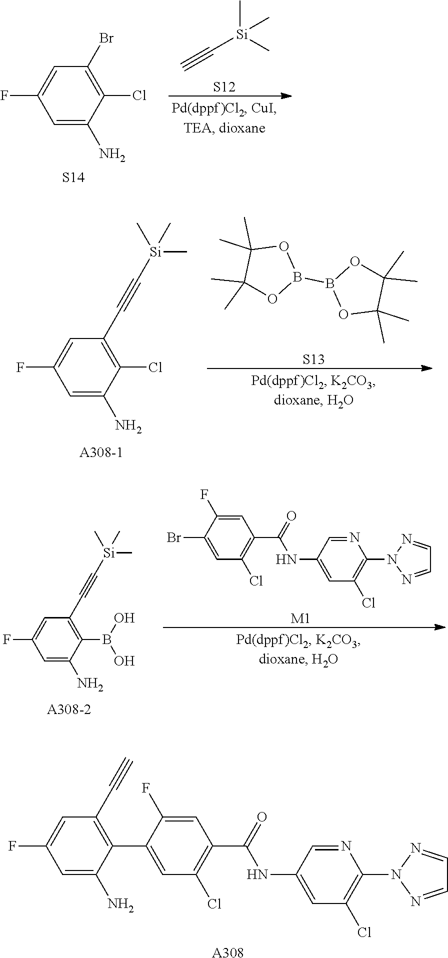

### Step 1: Synthesis of Compound A308-1

The mixture of S14 (443 mg), S12 (300 mg), Pd(PPh₃)₂Cl₂ (3 mg), CuI (5 mg), TEA (1 mL) and dioxane (10 mL) was stirred at 90° C. for 8 hours under nitrogen. The mixture was cooled to room temperature, and diluted with EA (100 mL) and washed with brine. The organic phase was concentrated and purified by flash column chromatography (DCM/MeOH=40/1) to give compound A308-1 (240 mg) as colorless oil. LC-MS (ES⁺): m/z 242[M+H]⁺

### Step 2: Synthesis of Compound A308-2

The mixture of A308-1 (240 mg), S13 (500 mg), Pd(dppf)Cl₂ (30 mg), K₂CO₃ (270 mg), dioxane (10 mL) and H₂O (2 mL) was stirred at 110° C. for 8 hours under nitrogen. The mixture was cooled to room temperature, diluted with EA (100 mL) and washed with brine. The organic phase was concentrated and purified by flash column chromatography (DCM/MeOH=40/1) to give compound A308-2 (101 mg) as yellow oil. LC-MS (ES⁺): m/z 252[M+H]⁺

### Step 3: Synthesis of Compound A308

The mixture of M1 (43 mg), A308-2 (37 mg), Pd(dppf)Cl₂ (3 mg), K₂CO₃ (27 mg), dioxane (1 mL) and H₂O (0.2 mL) was stirred at 90° C. for 8 hours under nitrogen. The mixture was cooled to room temperature, diluted with EA (5 mL) and washed with brine. The organic phase was concentrated and purified by flash column chromatography (DCM/MeOH=40/1) to give compound A308 (19 mg) as yellow solid. LC-MS (ES⁺): m/z 485[M+H]⁺

### Example 310: Synthesis of Compound A310

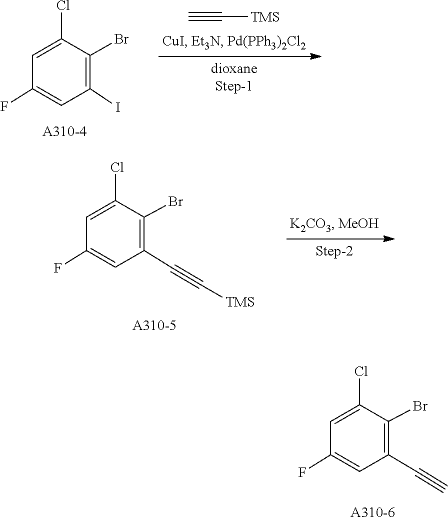

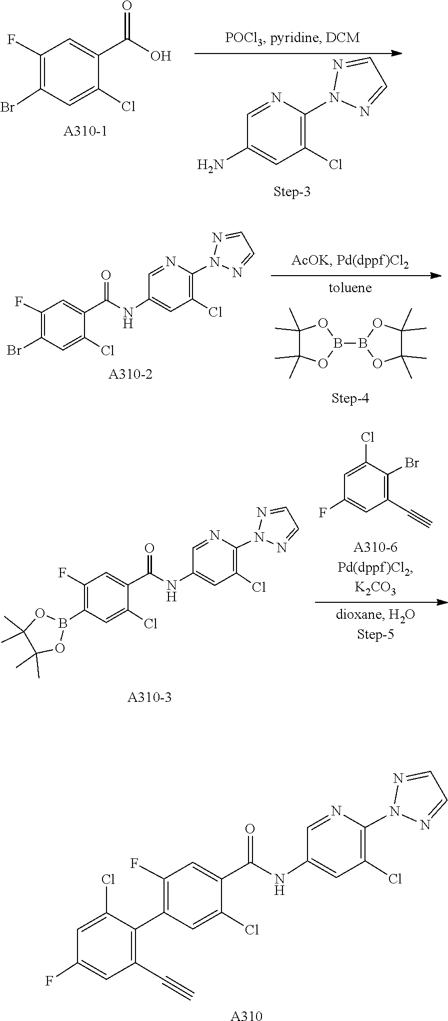

### Step 1: Synthesis of Compound A310-5

To a solution of A310-4 (3.33 g, 10.0 mmol) in dioxane (35 mL) was added CuI (0.38 g, 2.0 mmol), Et₃N (3.03 g, 30.0 mmol), Pd(PPh₃)₂Cl₂ (0.73 g, 1 mmol) and ethyltrimethylsilane (2.04 g, 20.0 mmol). The reaction mixture was stirred at room temperature under nitrogen for 5 hours and quenched by water, extracted with dichloromethane (250 mLxa). The organic layer was washed by brine, dried over sodium sulfate, filtrated and evaporated in vacuo. The residue was purified by column chromatography to give compound A310-5 (2.5 g) as yellow solid. LC-MS (ES⁺): m/z 305[M+H]⁺

### Step 2: Synthesis of Compound A310-6

To a solution of A310-5 (2.5 g, 8.3 mmol) in methanol (30 mL) was added K₂CO₃ (3.45 g, 24.9 mmol). The reaction mixture was stirred at room temperature under nitrogen for 2 hours. Removing the solvent and purifying by column chromatography to give compound A310-6 (1.7 g) as Off-white solid. LC-MS (ES⁺): m/z 233[M+H]⁺

### Step 3: Synthesis of Compound A310-2

To a solution of A310-1 (2.52 g, 10 mmol) in DCM (30 mL) was added pyridine (2.37 g, 30 mmol) and 5-chloro-6-(2H-1,2,3-triazol-2-yl)pyridin-3-amine (1.95 g, 10 mmol). Then POCl₃ (1.68 g, 11 mmol) was added dropwise. The reaction was stirred at room temperature under nitrogen for 2 hours. The reaction was quenched by water and extracted by DCM (100 mL×2). The organic layer was concentrated and purified by column chromatography to give compound A310-2 (3.9 g) as off-white solid. LC-MS (ES⁺): m/z 430[M+H]⁺

### Step 4: Synthesis of Compound A310-3

The mixture of A310-2 (3.9 g, 9.0 mmol), potassium acetate (2.65 g, 27.0 mmol) Pd(dppf)Cl₂ (658 mg, 0.9 mmol), and pinacol diborate (3.71 g, 14.5 mmol) in toluene (50 mL), was stirred at 75° C. under nitrogen for 2 hours. Removing the solvent and purifying by column chromatography to give compound A310-3 (3.7 g) as off-white solid LC-MS (ES⁺): m/z 478[M+H]⁺

### Step 5: Synthesis of Compound A310

The mixture of A310-3 (95 mg, 0.2 mmol), K₂CO₃ (83 mg, 0.6 mmol), Pd(dppf)Cl₂ (15 mg, 0.02 mmol), A310-6 (46 mg, 0.2 mmol), dioxane (1.5 mL) and H₂O (0.3 mL) was stirred at 75° C. under nitrogen for 2 hours. The reaction was concentrated and purified by column chromatography to give compound A310 (35 mg) as off-white solid. LC-MS (ES⁺): m/z 504[M+H]⁺

### Example 315: Synthesis of Compound A315

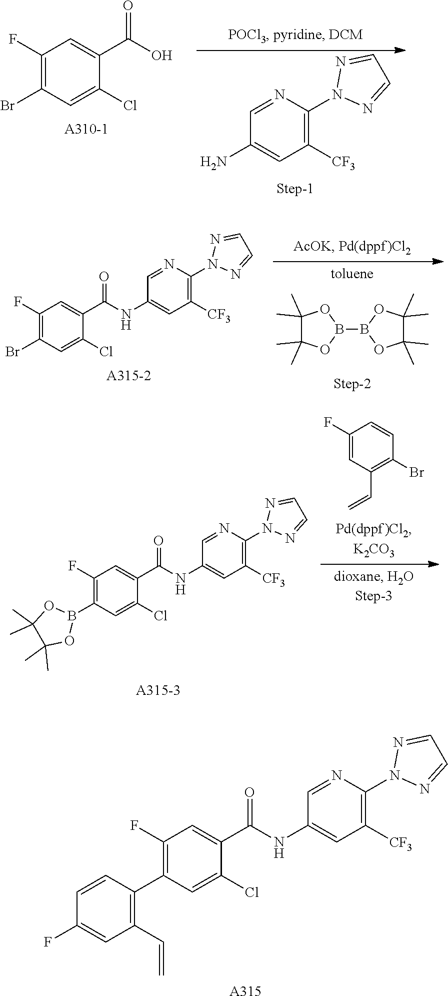

### Step 1: Synthesis of Compound A315-2

To a solution of A315-2 (2.52 g, 10 mmol) in DCM (30 mL), was added pyridine (2.37 g, 30 mmol) and 6-(2H-1,2,3-triazol-2-yl)-5-(trifluoromethyl)pyridin-3-amine (2.29 g, 10 mmol). Then POCl₃ (1.68 g, 11 mmol) was added dropwise. The reaction was stirred at room temperature for 2 hours. The reaction was quenched by water and extracted by DCM (200 mL×2), the organic layer was concentrated in vacuum and purified by column chromatography to afford compound A315-2 (3.9 g) as off-white solid. LC-MS (ES⁺): m/z 464[M+H]⁺

### Step 2: Synthesis of Compound A315-3

The mixture of A310-2 (3.9 g, 8.4 mmol), potassium acetate (2.48 g, 25.2 mmol), Pd(dppf)Cl₂ (585 mg, 0.8 mmol) and pinacol diborate (3.28 g, 12.8 mmol) in toluene (50 mL) was stirred at 75° C. under nitrogen for 2 hours. Removing the solvent and purifying by column chromatography to afforded compound A315-3 (3.7 g) as off-white solid. LC-MS (ES⁺): m/z 512[M+H]⁺

### Step 3: Synthesis of Compound A315

The mixture of A310-3 (102 mg, 0.2 mmol), K₂CO₃ (83 mg, 0.6 mmol), Pd(dppf)Cl₂ (15 mg, 0.02 mmol), 1-bromo-4-fluoro-2-vinylbenzene (40 mg, 0.2 mmol), dioxane (1.5 mL) and H₂O (0.3 mL) was stirred at 75° C. under nitrogen for 2 hours. The reaction was concentrated and purified by column chromatography to give compound A315 (30 mg) as off-white solid. LC-MS (ES⁺): m/z 504[M+H]⁺

### Example 316: Synthesis of Compound A316

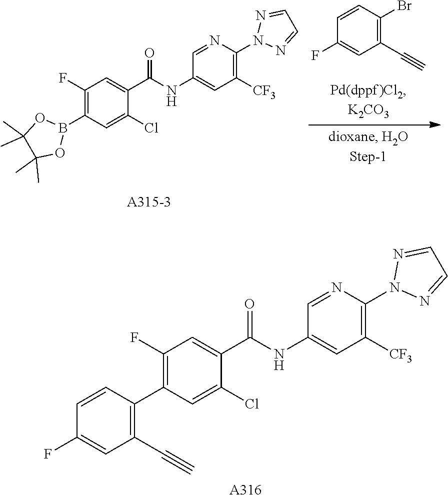

The mixture of A315-3 (102 mg, 0.2 mmol), K₂CO₃ (83 mg, 0.6 mmol), Pd(dppf)Cl₂ (15 mg, 0.02 mmol), 1-bromo-2-ethynyl-4-fluorobenzene (40 mg, 0.2 mmol), dioxane (1.5 mL) and H₂O (0.3 mL) was stirred at 75° C. under nitrogen for 2 hours. The reaction was concentrated and purified by column chromatography to give compound A316 (30 mg) as off-white solid. LC-MS (ES⁺): m/z 504[M+H]⁺

### Example 318: Synthesis of Compound A318

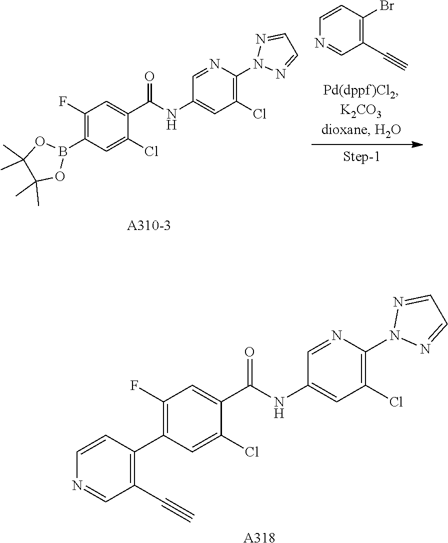

The mixture of A310-3 (95 mg, 0.2 mmol), K₂CO₃ (83 mg, 0.6 mmol), Pd(dppf)Cl₂ (15 mg, 0.02 mmol), 4-bromo-3-ethynylpyridine (36 mg, 0.2 mmol), dioxane (1.5 mL) and H₂O (0.3 mL) was stirred at 75° C. under nitrogen for 2 hours. The reaction was concentrated and purified by column chromatography to give Compound A318 (36 mg) as off-white solid. LC-MS (ES⁺): m/z 453[M+H]⁺

### Example 319: Synthesis of Compound A319

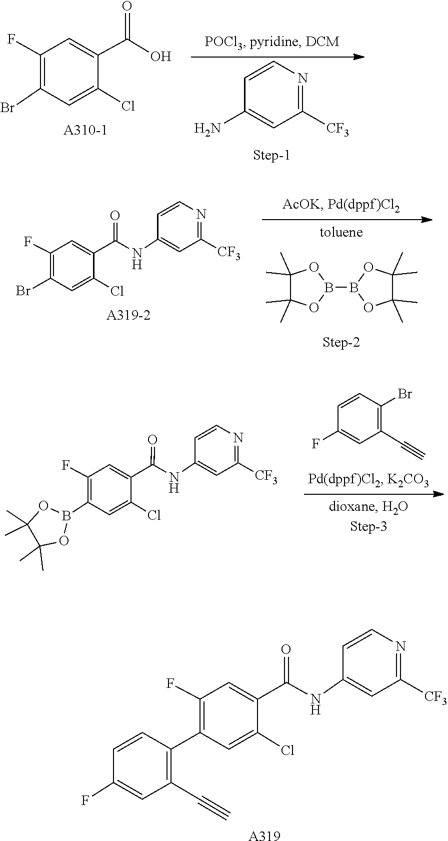

### Step 1: Synthesis of Compound A319-2

To a solution of A310-1 (2.52 g, 10 mmol) in DCM (30 mL), was added pyridine (2.37 g, 30 mmol) and 5-(trifluoromethyl)pyridin-3-amine (1.62 g, 10 mmol). Then POCl₃ (1.68 g, 11 mmol) was added dropwise. The reaction was stirred at room temperature for 2 hours. The reaction was quenched by water and extracted by DCM (100 mL×2), the organic layer was concentrated and purified by column chromatography to give compound A319-2 (3.1 g) as off-white solid. LC-MS (ES⁺): m/z 397[M+H]⁺

### Step 2: Synthesis of Compound A319-3

The mixture of A319-2 (3.1 g, 7.8 mmol), potassium acetate (2.30 g, 23.5 mmol), Pd(dppf)Cl₂ (585 mg, 0.8 mmol), and pinacol diborate (3.00 g, 11.7 mmol) in toluene (35 mL), was stirred at 75° C. under nitrogen for 2 hours. The reaction was concentrated and purified by column chromatography to give compound A319-3 (3.7 g) as off-white solid. LC-MS (ES⁺): m/z 445[M+H]⁺

### Step 3: Synthesis of Compound A319

The mixture of A319-3 (90 mg, 0.2 mmol), K₂CO₃ (83 mg, 0.6 mmol), Pd(dppf)Cl₂ (15 mg, 0.02 mmol), 1-bromo-2-ethynyl-4-fluorobenzene (40 mg, 0.2 mmol), dioxane (1.5 mL) and H₂O (0.3 mL) was stirred at 75° C. under nitrogen for 2 hours. The reaction was concentrated in vacuum and purified by column chromatography to give compound A319 (33 mg) as off-white solid. LC-MS (ES⁺): m/z 437[M+H]⁺

### Example 323: Synthesis of Compound A323

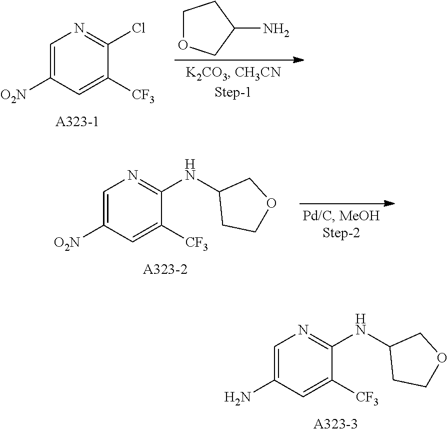 [SMILES: O=[N+]([O-])c1cnc(Cl)c(C(F)(F)F)c1 ; NC1CCOC1 ; O=[N+]([O-])c1cnc(NC2CCOC2)c(C(F)(F)F)c1 ; Nc1cnc(NC2CCOC2)c(C(F)(F)F)c1]

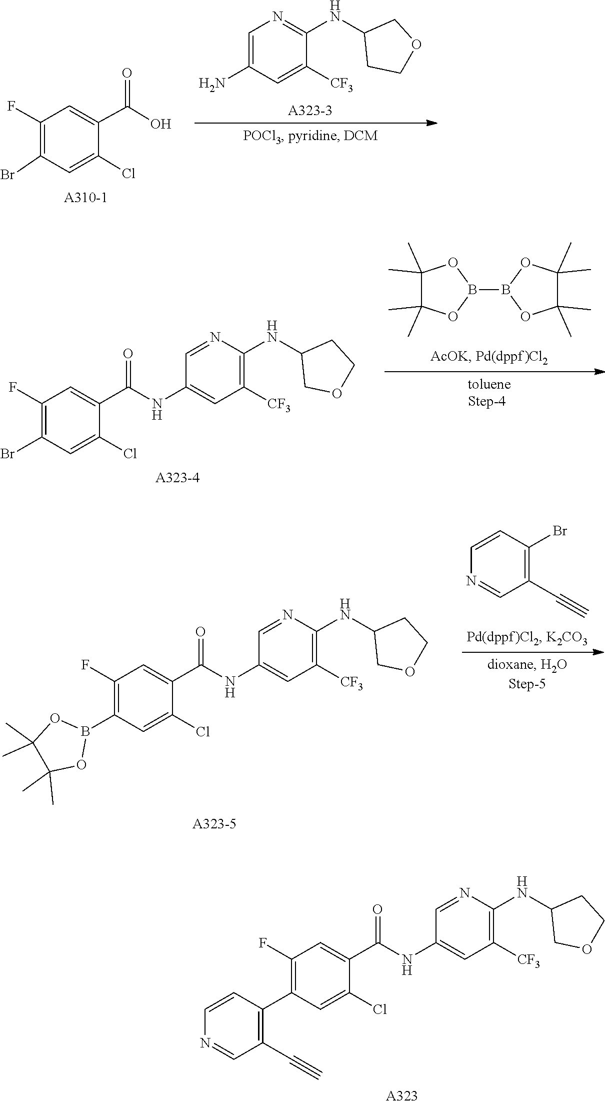

### Step 1: Synthesis of Compound A323-2

To a solution of A323-1 (2.26 g, 10 mmol) in acetonitrile (30 mL) was added potassium carbonate (4.14 g, 30 mmol) and tetrahydrofuran-3-amine (1.75 g, 20 mmol). The reaction was stirred at 75° C. under nitrogen for 2 hours. The reaction was concentrated and purified by column chromatography to give compound A323-2 (2.3 g) as off-white solid. LC-MS (ES⁺): m/z 278[M+H]⁺

### Step 2: Synthesis of Compound A323-3

To a solution of A323-2 (2.26 g, 8.2 mmol) in methanol (30 mL) was added Pd/C (10%, 435 mg) under nitrogen atmosphere in a 100 mL round bottom flask. The flask was then vacuumed and flushed with hydrogen. The reaction mixture was hydrogenated at 75° C. for 2 hours under hydrogen atmosphere using a hydrogen balloon, then filtered through a Celite pad and concentrated under reduced pressure. The residue was purified by column chromatography to afford compound A323-3 (1.8 g) as off-white solid. LC-MS (ES⁺): m/z 248[M+H]+

### Step 3: Synthesis of Compound A323-4

To a solution of A310-1 (1.8 g, 7.2 mmol) in DCM (30 mL) was added pyridine (1.71 g, 21.6 mmol), N²-(tetrahydrofuran-3-yl)-3-(trifluoromethyl)pyridine-2,5-diamine (1.41 g, 7.2 mmol) and POCl₃ (1.21 g, 7.2 mmol). The reaction was stirred at room temperature for 2 hours. The reaction was quenched by water and extracted by DCM (100 mL×2), the organic layer was concentrated and purified by column chromatography to give compound A323-4 (3.1 g) as off-white solid. LC-MS (ES⁺): m/z 482[M+H]⁺

### Step 4: Synthesis of Compound A323-5

The mixture of A323-4 (3.1 g, 6.4 mmol), potassium acetate (2.47 g, 15.2 mmol), Pd(dppf)Cl₂ (439 mg, 0.6 mmol) and pinacol diborate (2.45 g, 9.6 mmol) in toluene (35 mL) was stirred at 75° C. under nitrogen for 2 hours. The reaction was concentrated and purified by column chromatography to give A323-5 (3.1 g) as off-white solid. LC-MS (ES⁺): m/z 530[M+H]+

### Step 5: Synthesis of Compound A323

The mixture of A323-5 (106 mg, 0.2 mmol), K₂CO₃ (83 mg, 0.6 mmol), Pd(dppf)Cl₂ (15 mg, 0.02 mmol), 4-bromo-3-ethynylpyridine (36 mg, 0.2 mmol), dioxane (1.5 mL) and H₂O (0.3 mL) was stirred at 75° C. under nitrogen for 2 hours. The reaction solution was concentrated and purified by column chromatography to give compound A323 (37 mg) as off-white solid. LC-MS (ES⁺): m/z 505[M+H]⁺

### Example 327: Synthesis of Compound A327

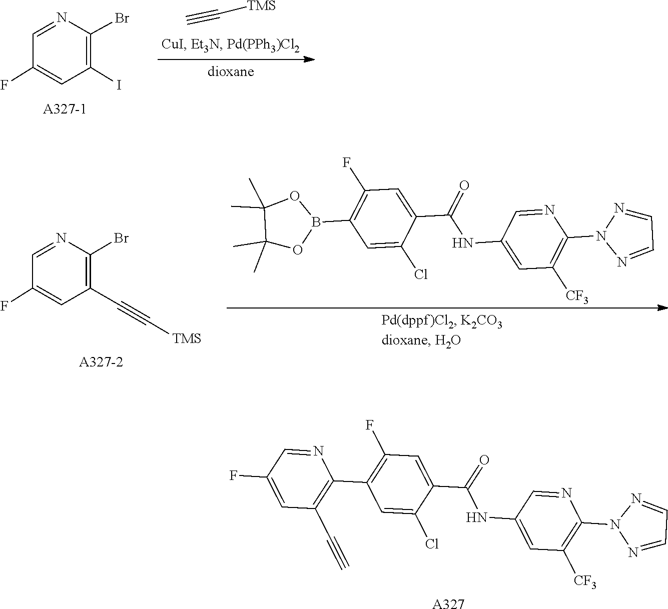

### Step 1: Synthesis of Compound A327-2

The mixture of A327-1 (3.01 g, 10.0 mmol), CuI (0.38 g, 2.0 mmol), Et₃N (3.03 g, 30.0 mmol), Pd(PPh₃)₂Cl₂ (0.73 g, 1 mmol), dioxane (35 mL) and ethyltrimethylsilane (2.04 g, 20.0 mmol) was stirred at room temperature under nitrogen for 5 hours. The reaction was quenched by water, extracted with dichloromethane (250 mL×2). The organic phase was dried over sodium sulfate, concentrated in vacuum and purified by column chromatography to give compound A327-2 (2.5 g) as yellow solid. LC-MS (ES⁺): m/z 272[M+H]⁺

### Step 2: Synthesis of Compound A327

The mixture of A315-3 (102 mg, 0.2 mmol), K₂CO₃ (83 mg, 0.6 mmol), Pd(dppf)Cl₂ (15 mg, 0.02 mmol), A327-2 (54 mg, 0.2 mmol), dioxane (1.5 mL) and H₂O (0.3 mL) was stirred at 75° C. under nitrogen for 2 hours. The reaction solution was concentrated in vacuum and purified by column chromatography to give compound A327 (38 mg) as off-white solid. LC-MS (ES⁺): m/z 505[M+H]⁺

### Example 331: Synthesis of Compound A331

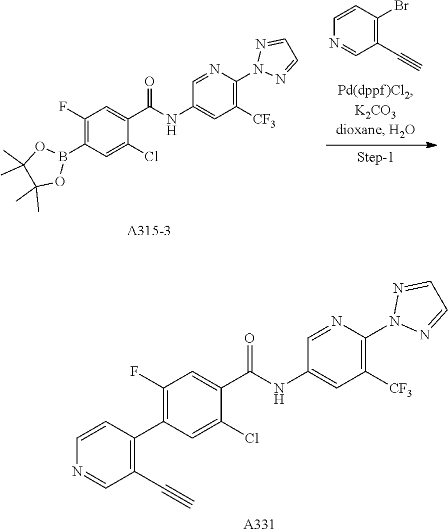

The mixture of A315-3 (95 mg, 0.2 mmol), K₂CO₃ (83 mg, 0.6 mmol), Pd(dppf)Cl₂ (15 mg, 0.02 mmol), 4-bromo-3-ethynylpyridine (36 mg, 0.2 mmol), dioxane (1.5 mL) and H₂O (0.3 mL) was stirred at 75° C. under nitrogen for 2 hours. The reaction solution was concentrated in vacuum and purified by column chromatography to give compound A331 (37 mg) as off-white solid. LC-MS (ES⁺): m/z 487[M+H]⁺

### Example 332: Synthesis of Compound A332

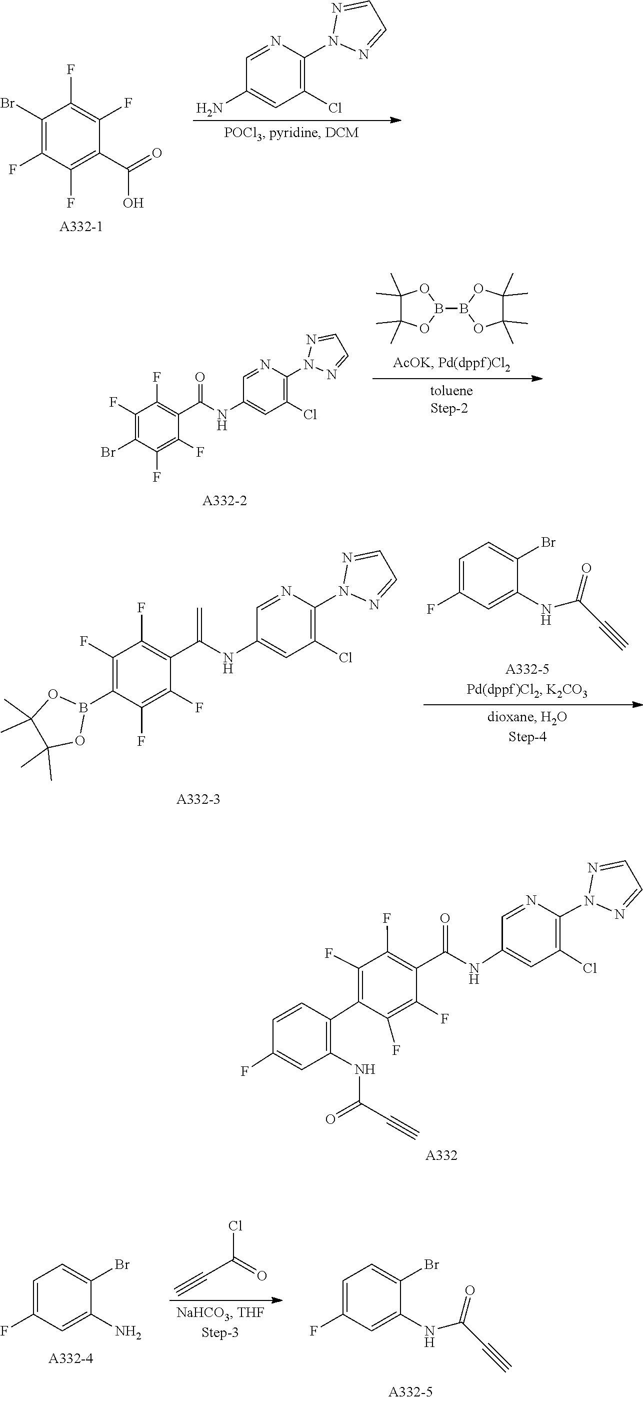

### Step 1: Synthesis of Compound A332-2

To a solution of A332-1 (2.72 g, 10 mmol) in DCM (30 mL) was added pyridine (2.37 g, 30 mmol), 5-chloro-6-(2H-1,2,3-triazol-2-yl)pyridin-3-amine (1.95 g, 10 mmol) and POCl₃ (1.68 g, 11 mmol). The reaction mixture was stirred at room temperature for 2 hours, quenched by water and extracted by DCM (100 mL×2). The organic layer was concentrated and purified by column chromatography to give compound A332-2 (3.9 g) as off-white solid. LC-MS (ES⁺): m/z 450[M+H]⁺

### Step 2: Synthesis of Compound A332-3

The mixture of A310-2 (3.9 g, 8.6 mmol), potassium acetate (2.53 g, 25.8 mmol), Pd(dppf)Cl₂ (658 mg, 0.9 mmol), pinacol diborate (3.31 g, 12.9 mmol) and toluene (50 mL) was stirred at 75° C. under nitrogen for 2 hours. The reaction solution was concentrated and purified by column chromatography to give Compound A332-3 (3.7 g) as off-white solid. LC-MS (ES⁺): m/z 501[M+H]⁺

### Step 3: Synthesis of Compound A332-5

To a solution of A332-4 (1.89 g, 10.0 mmol), in THF (30 mL) was added sodium bicarbonate (2.52 g, 30 mmol) and propargyl chloride (0.89 g, 10.0 mmol). The reaction was stirred at room temperature for 2 hours. The reaction was quenched by water and extracted by EA (100 mL×2), the organic layer was concentrated and purified by column chromatography to obtain compound A332-5 (1.7 g) as off-white solid. LC-MS (ES⁺): m/z 242[M+H]⁺

### Step 4: Synthesis of Compound A332

The mixture of A332-3 (100 mg, 0.2 mmol), K₂CO₃ (83 mg, 0.6 mmol), Pd(dppf)Cl₂ (15 mg, 0.02 mmol), A332-5 (48 mg, 0.2 mmol), dioxane (1.5 mL) and H₂O (0.3 mL) was stirred at 75° C. under nitrogen for 2 hours. The reaction was concentrated in vacuum and purified by column chromatography to give compound A332 (35 mg) as off-white solid. LC-MS (ES⁺): m/z 513[M+H]⁺

### Example 343: Synthesis of Compound A343

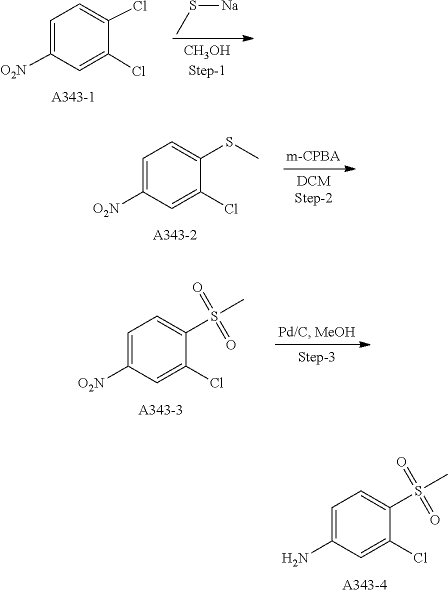

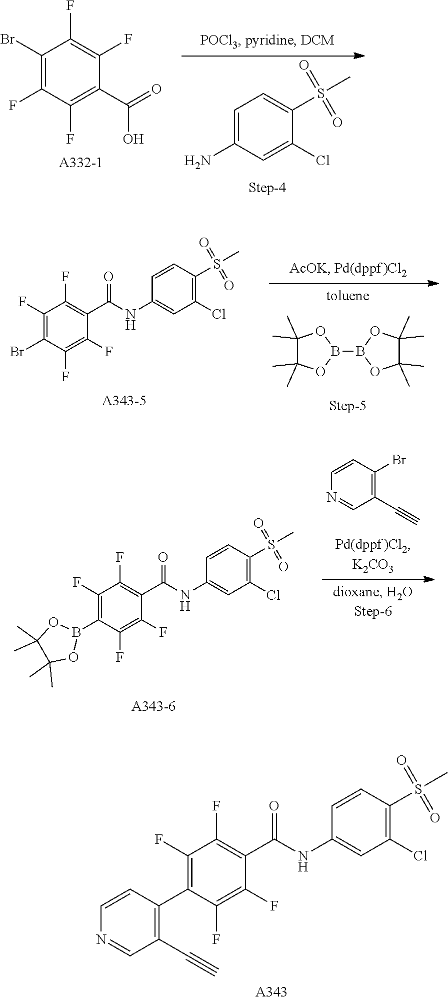

### Step 1: Synthesis of Compound A343-2

To a solution of A343-1 (1.91 g, 10.0 mmol) in methanol (25 mL) was added sodium methylmercaptide (1.40 g, 20 mmol). The reaction was stirred at 75° C. for 2 hours. The reaction solution was concentrated and purified by column chromatography to give off-white solid A343-2 (1.5 g). LC-MS (ES⁺): m/z 204[M+H]⁺

### Step 2: Synthesis of Compound A343-3

To a solution of A343-2 (1.50 g, 7.3 mmol) in dichloromethane (20 mL) was added m-CPBA (1.73 g, 10 mmol). The reaction was stirred at room temperature for 2 hours. Monitored by LCMS, then concentrated and purified by column chromatography to give off-white solid A343-3 (1.1 g). LC-MS (ES⁺): m/z 236[M+H]⁺

### Step 3: Synthesis of Compound A343-4

A343-3 (1.10 g, 4.7 mmol) was added to the reaction flask, and then palladium on carbon (212 mg, 2 mmol) and methanol (15 mL) were added. The reaction was stirred at room temperature under hydrogen for 2 hours. The end of the reaction were monitored by LCMS. The reaction solution was filtered, evaporated to obtain off-white solid 900 mg. LC-MS (ES⁺): m/z 206[M+H]⁺.

### Step 4: Synthesis of Compound A343-5

A332-1 (2.72 g, 10 mmol) was added to the reaction flask, and then DCM (30 mL), pyridine (2.37 g, 30 mmol) and 3-chloro-4-methylsulfonylanilide (2.06 g, 10 mmol) were added. Then POCl₃ (1.68 g, 11 mmol) was added dropwise. The reaction was carried out at room temperature for 2 hours. The end of the reaction were monitored by LCMS. The reaction solution was purified by column chromatography to obtain off-white solid product 3.9 g. LC-MS (ES⁺): m/z 460[M+H]⁺

### Step 5: Synthesis of Compound A343-6

A343-5 (3.9 g, 8.5 mmol) was added to the reaction flask, and then potassium acetate (2.50 g, 25.5 mmol), Pd(dppf)Cl₂ (658 mg, 0.9 mmol), toluene (50 mL), pinacol diborate (3.31 g, 12.9 mmol) were added. The reaction was carried out at 75° C. under nitrogen for 2 hours. The end of the reaction were monitored by LCMS. The reaction solution was purified by column chromatography to obtain off-white solid product 3.7 g. LC-MS (ES⁺): m/z 508[M+H]⁺

### Step 6: Synthesis of Compound A343

A343-6 (114 mg, 0.2 mmol) was added to the reaction flask, and then K₂CO₃ (83 mg, 0.6 mmol), Pd(dppf)Cl₂ (15 mg, 0.02 mmol), 4-bromo-3-ethynylpyridine (36 mg, 0.2 mmol), dioxane (1.5 mL) and H₂O (0.3 mL) were added. The reaction was carried out at 75° C. under nitrogen for 2 hours. The end of the reaction were monitored by LCMS. The reaction solution was purified by column chromatography to obtain off-white solid product 39 mg. LC-MS (ES⁺): m/z 483[M+H]⁺

### Example 344: Synthesis of Compound A344

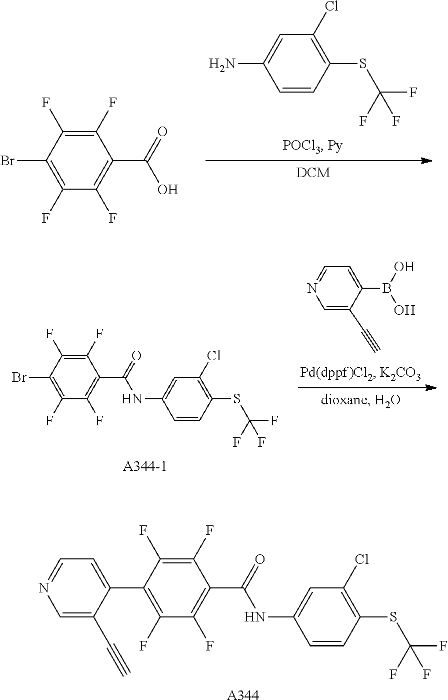 [SMILES: O=C(O)c1c(F)c(F)c(Br)c(F)c1F ; Nc1ccc(SC(F)(F)F)c(Cl)c1 ; O=C(Nc1ccc(SC(F)(F)F)c(Cl)c1)c1c(F)c(F)c(Br)c(F)c1F ; C#Cc1cnccc1B(O)O ; C#Cc1cnccc1-c1c(F)c(F)c(C(=O)Nc2ccc(SC(F)(F)F)c(Cl)c2)c(F)c1F ; "A344"]

### Step 1: Synthesis of Compound A344-1

4-bromo-2,3,5,6-tetrafluorobenzoic acid (92 mg), 3-chloro-4-((trifluoromethyl)thio) aniline (72 mg), pyridine (180 mg) and DCM (10 mL) were added to the 50 mL reaction flask, the solution was cooled to 0° C. then POCl₃ (200 mg) was added dropwise. the reaction was carried out at room temperature for 2 hour The reaction solution was poured into ice water, and dichloromethane (50 mL) was added to the above solution. The reaction solution was washed with saturated NaCl solution. The organic phase was purified by silica gel column (PE/EA=1/1) to obtain product A344-1 (55 mg), LC-MS (ES⁺): m/z 481[M+H]+

### Step 2: Synthesis of Compound A344

A344-1 (55 mg), (3-ethynylpyridin-4-yl)boronic acid (40 mg), Pd(dppf)Cl₂ (5 mg), K₂CO₃ (80 mg), dioxane (10 mL) and H₂O (1 mL) were added to a 25 mL round flask and replaced with nitrogen. The reaction mixture was heated to 90° C. and reacted for 4 hours. The end of the reaction were monitored by LCMS. The reaction solution was diluted with EA (5 mL), washed with saturated NaCl solution. The organic phase was concentrated and purified by silica gel column (PE/EA=1/1) to obtain product A344 (16 mg), LC-MS (ES⁺): m/z 505[M+H]⁺.

### Example 361: Synthesis of Compound A361

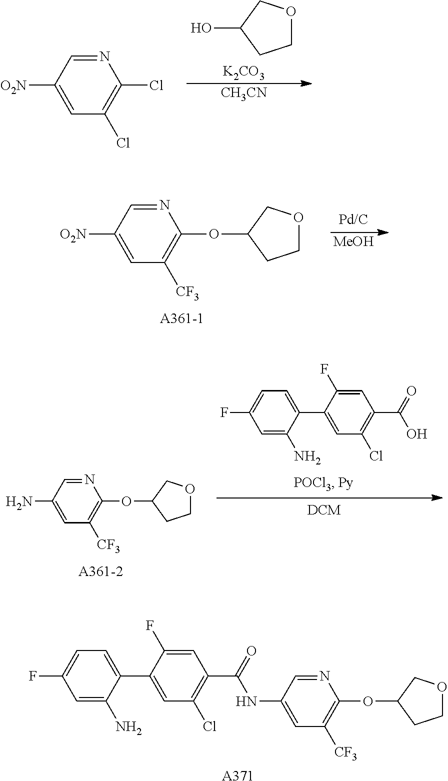

### Step 1: Synthesis of Compound A361-1

2,3-dichloro-5-nitropyridine (400 mg), tetrahydrofuran-3-ol (160 mg), potassium carbonate (552 mg) and ethiperidine (20 mL) were added to a 25 mL round flask and heated to 80° C. and reacted for 3 hours. The reaction solution was cooled to room temperature and filtered. The reaction solution was diluted with EA (200 mL), washed with saturated NaCl solution. The organic phase was concentrated and purified by silica gel column (PE/EA=1/1) to obtain product A361-1 (110 mg), LC-MS (ES⁺): m/z 279 [M+H]⁺

### Step 2: Synthesis of Compound A361-2

A361-1 (110 mg), ethanol (10 mL) and Pd/C (40 mg) were added to a 50 mL round flask and replaced with hydrogen three times. The reaction was stirred at room temperature under hydrogen for 1 hours. The reaction solution was filtered and concentrated to obtain product A361-2 (60 mg), LC-MS (ES⁺): m/z 249[M+H]⁺

### Step 3: Synthesis of Compound A361

A361-2 (50 mg), 2′-amino-5-chloro-2,4′-difluoro-[1,1′-biphenyl]-4-carboxylic acid (48 mg), pyridine (90 mg) and DCM (10 mL) were added to a 50 mL reaction flask, and cooled to 0° C. And POCl₃ (100 mg) was added dropwise. the reaction was carried out at room temperature for 2 hour. The reaction solution was poured into ice water. The reaction solution was diluted with DCM (50 mL), washed with saturated NaCl solution. The organic phase was concentrated and purified by silica gel column (PE/EA=1/1) to obtain product A361 (13 mg), LC-MS (ES⁺): m/z 514[M+H]⁺

### Example 371: Synthesis of Compound A371

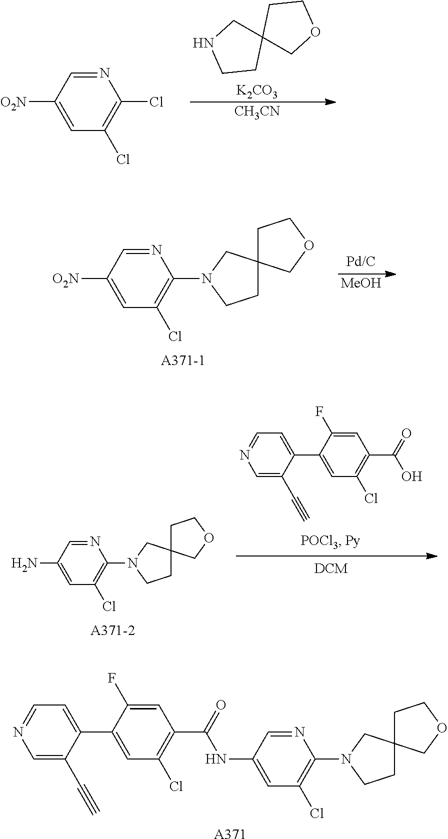

### Step 1: Synthesis of Compound A371-1

2,3-dichloro-5-nitropyridine (400 mg), 2-oxo-7-azaspiro[4.4]nonane (250 mg), potassium carbonate (552 mg) and acetonitrile (20 mL) were added to a 50 mL reaction flask, and heated to 80° C. and reacted for 3 hours. The reaction solution was cooled to room temperature and filtered. The reaction solution was diluted with EA (200 mL), washed with saturated NaCl solution twice. The organic phase was concentrated and purified by silica gel column (PE/EA=1/1) to obtain product A371-1 (300 mg), LC-MS (ES⁺): m/z 284 [M+H]⁺

### Step 2: Synthesis of Compound A371-2

A371-1 (160 mg), ethanol (10 mL) and Pd/C (50 mg) were added to a 50 mL reaction flask and replaced with hydrogen three times. The reaction was stirred at room temperature under hydrogen for 1 hours. The reaction solution was filtered and concentrated to obtain product A371-2 (60 mg), LC-MS (ES⁺): m/z 254[M+H]⁺

### Step 3: Synthesis of Compound A371

A371-2 (50 mg), 2-chloro-4-(3-ethynylpyridin-4-yl)-5-fluorobenzoic acid (76 mg), pyridine (100 mg) and DCM (10 mL) were added to a 50 mL reaction flask, and stirred and cooled to 0° C. And POCl₃ (105 mg) was added dropwise. the reaction was carried out at room temperature for 2 hour. The reaction solution was poured into ice water to quench the reaction. The reaction solution was diluted with DCM (50 mL), washed with saturated NaCl solution. The organic phase was concentrated and purified by silica gel column (PE/EA=1/1) to obtain product A371 (23 mg), LC-MS (ES⁺): m/z 511[M+H]⁺

### Example 373: Synthesis of Compound A373

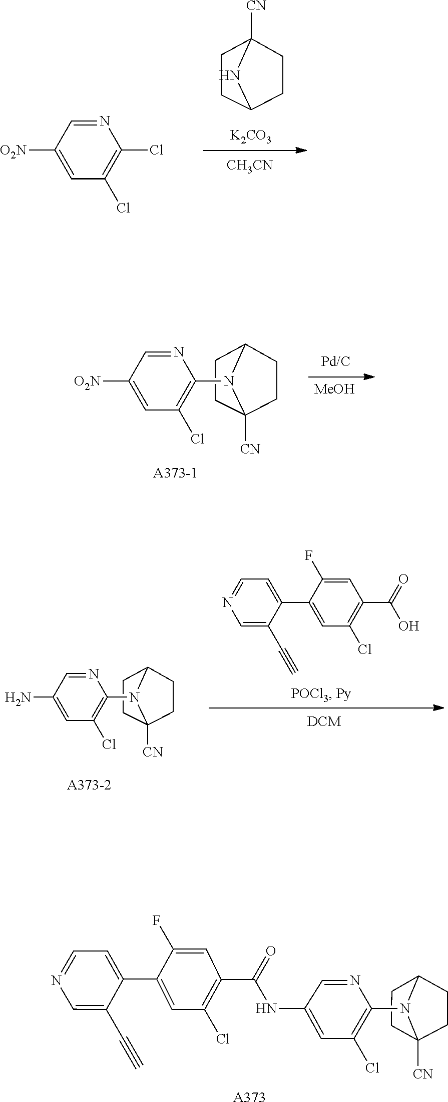

### Step 1: Synthesis of Compound A373-1

2,3-dichloro-5-nitropyridine (400 mg), 7-azabicyclo[2.2.1]heptane-1-carbonitrile (212 mg), potassium carbonate (552 mg) and acetonitrile (20 mL) were added to a 50 mL reaction flask, and heated to 80° C. and reacted for 3 hours. The reaction solution was cooled to room temperature and filtered. The reaction solution was diluted with EA (200 mL), washed with saturated NaCl solution twice. The organic phase was concentrated and purified by silica gel column (PE/EA=1/1) to obtain product A373-1 (120 mg), LC-MS (ES⁺): m/z 279 [M+H]⁺

### Step 2: Synthesis of Compound A373-2

A373-1 (120 mg), ethanol (10 mL) and Pd/C (40 mg) were added to a 50 mL reaction flask and replaced with hydrogen three times. The reaction was stirred at room temperature under hydrogen for 1 hours. The reaction solution was filtered and concentrated to obtain product A373-2 (40 mg), LC-MS (ES⁺): m/z 249[M+H]⁺

### Step 3: Synthesis of Compound A373

A373-2 (40 mg), 2-chloro-4-(3-ethynylpyridin-4-yl)-5-fluorobenzoic acid (45 mg), pyridine (70 mg), and DCM (10 mL) were added to a 50 mL reaction flask, and cooled to 0° C. And POCl₃ (75 mg) was added dropwise. the reaction was carried out at room temperature for 2 hour. The reaction solution was poured into ice water The reaction solution was diluted with DCM (50 mL), washed with saturated NaCl solution twice. The organic phase was concentrated and purified by silica gel column (PE/EA=1/1) to obtain product A373 (7 mg), LC-MS (ES⁺): m/z 506[M+H]⁺

### Example 374: Synthesis of Compound A374

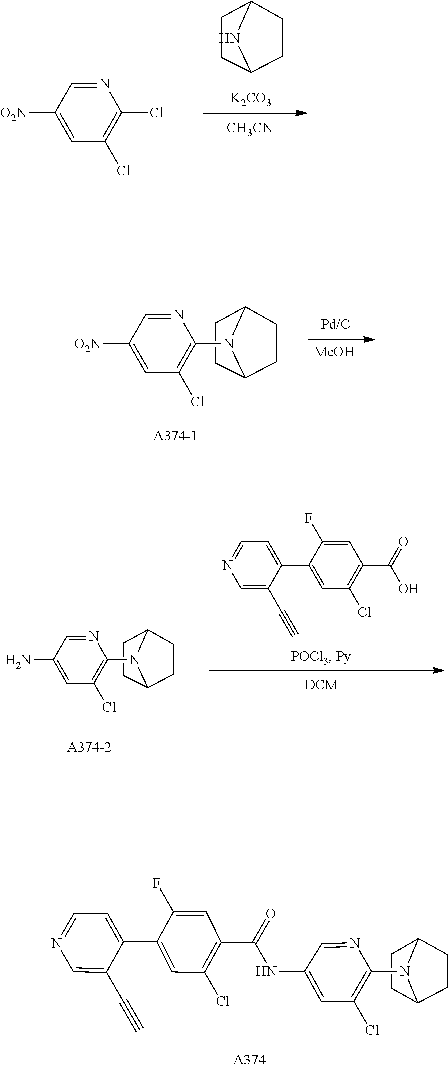

### Step 1: Synthesis of Compound A374-1

2,3-Dichloro-5-nitropyridine (400 mg), (7-azabicyclo[2.2.1]heptane (196 mg), potassium carbonate (552 mg) and acetonitrile (20 mL) were added to a 50 mL reaction flask, and heated to 80° C. and reacted for 3 hours. The reaction solution was cooled to room temperature and filtered. The filtrate was diluted with EA (200 mL), washed with saturated NaCl solution twice. The organic phase was concentrated and purified by silica gel column (PE/EA=1/1) to obtain product A374-1 (150 mg), LC-MS (ES⁺): m/z 254 [M+H]⁺

### Step 2: Synthesis of Compound A374-2

A374-1 (150 mg), ethanol (10 mL) and Pd/C (50 mg) were added to a 50 mL reaction flask and replaced with hydrogen three times. The reaction was stirred at room temperature under hydrogen for 1 hours. The reaction solution was filtered and concentrated to obtain product A374-2 (60 mg), LC-MS (ES⁺): m/z 224[M+H]⁺

### Step 3: Synthesis of Compound A374

A374-2 (40 mg), 2-chloro-4-(3-ethynylpyridin-4-yl)-5-fluorobenzoic acid (55 mg), pyridine (70 mg) and DCM (10 mL) were added to a 50 mL reaction flask, and stirred and cooled to 0° C. And POCl₃ (90 mg) was added dropwise. the reaction was carried out at room temperature for 2 hour. The reaction solution was poured into ice water The reaction solution was diluted with DCM (50 mL), washed twice with saturated NaCl solution. The organic phase was concentrated and purified by silica gel column (PE/EA=1/1) to obtain product A374 (6 mg), LC-MS (ES⁺): m/z 481[M+H]⁺

### Example 377: Synthesis of Compound A377

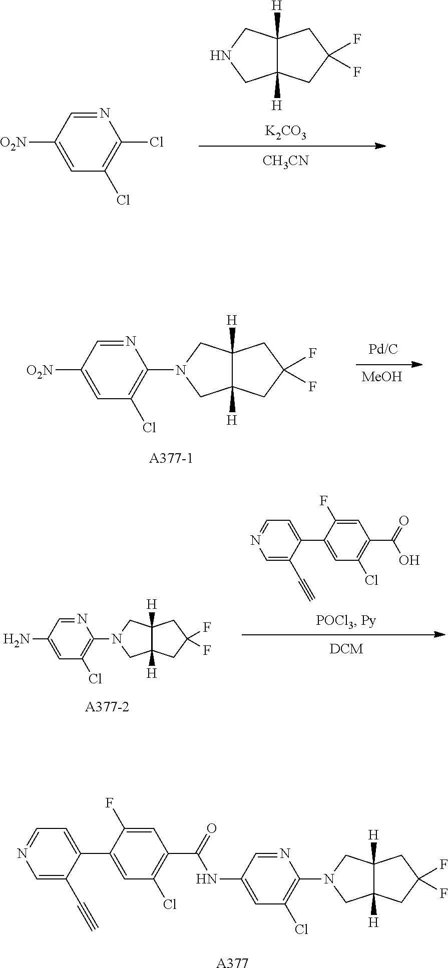

### Step 1: Synthesis of Compound A377-1

2,3-dichloro-5-nitropyridine (400 mg), (3aR,6aS)-5,5-difluorooctahydropenta[c]pyrrole (294 mg), potassium carbonate (552 mg) and acetonitrile (20 mL) were added to a 50 mL reaction flask, and heated to 80° C. and reacted for 3 hours. The reaction solution was cooled to room temperature and filtered. The reaction solution was diluted with EA (200 mL), washed with saturated NaCl solution twice. The organic phase was concentrated and purified by silica gel column (PE/EA=1/1) to obtain product A377-1 (180 mg), LC-MS (ES⁺): m/z 304 [M+H]⁺

### Step 2: Synthesis of Compound A377-2

A377-1 (150 mg), ethanol (10 mL) and Pd/C (50 mg) were added to a 50 mL reaction flask and replaced with hydrogen three times. The reaction was stirred at room temperature under hydrogen for 1 hours. The reaction solution was filtered and concentrated to obtain product A377-2 (70 mg), LC-MS (ES⁺): m/z 274[M+H]⁺

### Step 3: Synthesis of Compound A377

A377-2 (50 mg), 2-chloro-4-(3-ethynylpyridin-4-yl)-5-fluorobenzoic acid (55 mg), pyridine (70 mg) and DCM (10 mL) were added to a 50 mL reaction flask, and stirred and cooled to 0° C. And POCl₃ (90 mg) was added dropwise. the reaction was carried out at room temperature for 2 hour. The reaction solution was poured into ice water The reaction solution was diluted with DCM (50 mL), washed twice with saturated NaCl solution. The organic phase was concentrated and purified by silica gel column (PE/EA=1/1) to obtain product A377 (11 mg), LC-MS (ES⁺): m/z 531[M+H]⁺

### Example 381: Synthesis of Compound A381

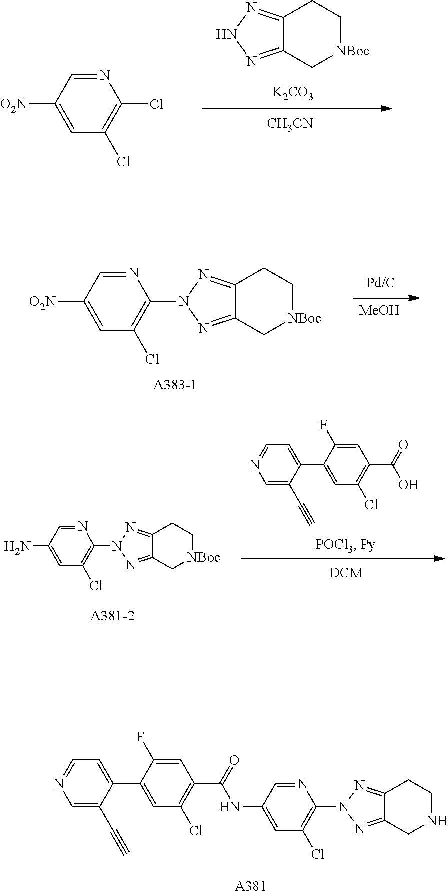

### Step 1: Synthesis of Compound A381-1

2,3-dichloro-5-nitropyridine (400 mg), 2,4,6,7-tetrahydro-5H-[1,2,3]triazolyl[4,5-c]pyridine-5-tert-butyl carboxylate (450 mg), potassium carbonate (552 mg) and acetonitrile (20 mL) were added to a 50 mL reaction flask, and heated to 80° C. and reacted for 3 hours. The reaction solution was cooled to room temperature and filtered. The reaction solution was diluted with EA (200 mL), washed with saturated NaCl solution twice. The organic phase was concentrated and purified by silica gel column (PE/EA=1/1) to obtain product A381-1 (406 mg), LC-MS (ES⁺): m/z 381 [M+H]⁺

### Step 2: Synthesis of Compound A381-2

A381-1 (200 mg), ethanol (10 mL) and Pd/C (100 mg) were added to a 50 mL reaction flask and replaced with hydrogen three times. The reaction was stirred at room temperature under hydrogen for 1 hour. The reaction solution was filtered and concentrated to obtain product A381-2 (110 mg), LC-MS (ES⁺): m/z 351[M+H]⁺

### Step 3: Synthesis of Compound A381

A381-2 (45 mg), 2-chloro-4-(3-ethynylpyridin-4-yl)-5-fluorobenzoic acid (50 mg), pyridine (70 mg) and DCM (10 mL) were added to a 50 mL reaction flask, and stirred and cooled to 0° C. And POCl₃ (80 mg) was added dropwise. the reaction was carried out at room temperature for 2 hour. The reaction solution was poured into ice water The reaction solution was diluted with DCM (50 mL), washed twice with saturated NaCl solution. The organic phase was concentrated and purified by silica gel column (PE/EA=1/1) to obtain product A381 (7 mg), LC-MS (ES⁺): m/z 508 [M+H]+

### Example 383: Synthesis of Compound A383

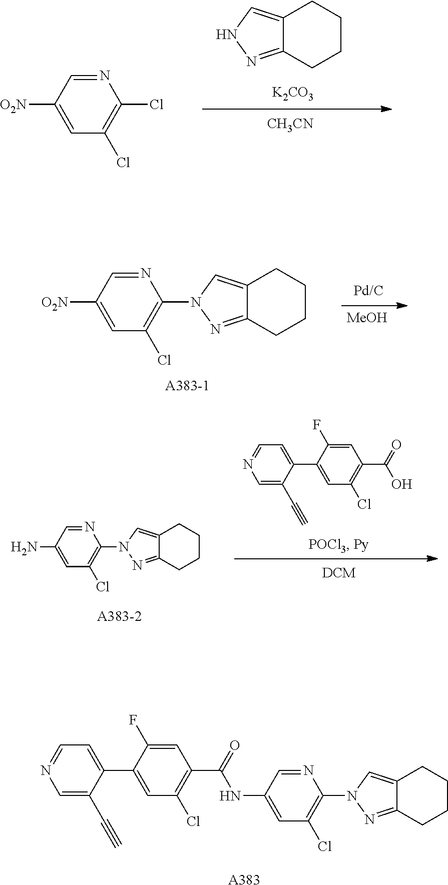

### Step 1: Synthesis of Compound A383-1

2,3-dichloro-5-nitropyridine (400 mg), 4,5,6,7-tetrahydro-2H-indazole (245 mg), potassium carbonate (552 mg) and acetonitrile (20 mL) were added to a 50 mL reaction flask, and heated to 80° C. and reacted for 3 hours. The reaction solution was cooled to room temperature and filtered. The reaction solution was diluted with EA (200 mL), washed with saturated NaCl solution twice. The organic phase was concentrated and purified by silica gel column (PE/EA=1/1) to obtain product A383-1 (310 mg), LC-MS (ES⁺): m/z 279 [M+H]⁺

### Step 2: Synthesis of Compound A383-2

A383-1 (310 mg), ethanol (10 mL) and Pd/C (100 mg) were added to a 50 mL reaction bottle and replaced with hydrogen three times. The reaction was stirred at room temperature under hydrogen for 1 hour. The reaction solution was filtered and concentrated to obtain product A383-2 (160 mg), LC-MS (ES⁺): m/z 249[M+H]⁺

### Step 3: Synthesis of Compound A383

A383-2 (45 mg, 0.18 mmol), 2-chloro-4-(3-ethynylpyridin-4-yl)-5-fluorobenzoic acid (50 mg, 0.18 mmol), pyridine (70 mg, 0.89 mmol) and DCM (10 mL) were added to a 50 mL reaction flask, and stirred and cooled to 0° C. And POCl₃ (80 mg, 0.52 mmol) was added dropwise. After the dripping, the reaction was stirred at room temperature for 2 hours. The reaction solution was poured into ice water to quench the reaction. The reaction solution was diluted with DCM (50 mL), washed twice with saturated NaCl solution. The organic phase was concentrated and separated by silica gel column (PE/EA=1/1) to obtain product A383 (11 mg, 0.022 mmol), LC-MS (ES⁺): m/z 506 [M+H]⁺

### Example 388: Synthesis of Compound A388

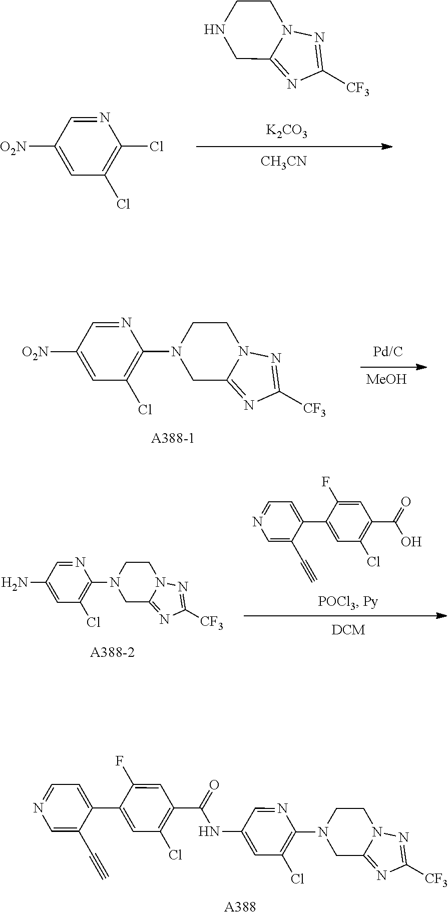

### Step 1: Synthesis of Compound A388-1

2,3-dichloro-5-nitropyridine (400 mg, 2.08 mmol), 2-(trifluoromethyl)-5,6,7,8-tetrahydro-[1,2,4]triazolo[1,5-a]pyrazine (400 mg, 2.08 mmol), potassium carbonate (552 mg, 4 mmol) and acetonitrile (20 mL) were added to a 50 mL reaction flask, and heated to 80° C. and reacted for 3 hours. The reaction solution was cooled to room temperature and filtered. The reaction solution was diluted with EA (200 mL), washed twice with saturated NaCl solution. The organic phase was concentrated and separated by silica gel column (PE/EA=1/1) to obtain product A388-1 (506 mg, 1.45 mmol), LC-MS (ES⁺): m/z 349 [M+H]⁺

### Step 2: Synthesis of Compound A388-2

A388-1 (480 mg, 1.38 mmol), MeOH (10 mL) and Pd/C (100 mg) were added to a 50 mL reaction bottle and replaced with hydrogen three times. Hydrogen was added and stirred for 1 hour at room temperature. The reaction solution was filtered and concentrated to obtain product A388-2 (300 mg, 0.94 mmol), LC-MS (ES⁺): m/z 319[M+H]⁺

### Step 3: Synthesis of Compound A388

A388-2 (50 mg, 0.16 mmol), 2-chloro-4-(3-ethynylpyridin-4-yl)-5-fluorobenzoic acid (43 mg, 0.16 mmol), pyridine (62 mg, 0.78 mmol) and DCM (10 mL) were added to a 50 mL reaction flask, and stirred and cooled to 0° C. And POCl₃ (80 mg, 0.52 mmol) was added dropwise. After the dripping, the reaction was stirred at room temperature for 2 h. The reaction solution was poured into ice water to quench the reaction. The reaction solution was diluted with DCM (50 mL), washed twice with saturated NaCl solution. The organic phase was concentrated and separated by silica gel column (PE/EA=1/1) to obtain product A388 (20 mg, 0.035 mmol), LC-MS (ES⁺): m/z 576 [M+H]⁺

### Example 392: Synthesis of Compound A392

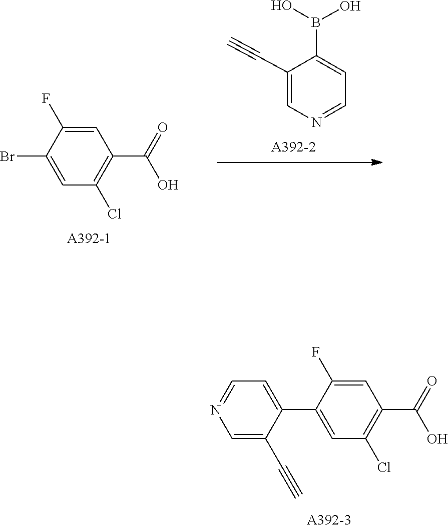 [SMILES: O=C(O)c1cc(F)c(Br)cc1Cl ; C#Cc1cnccc1B(O)O ; C#Cc1cnccc1-c1cc(Cl)c(C(=O)O)cc1F]

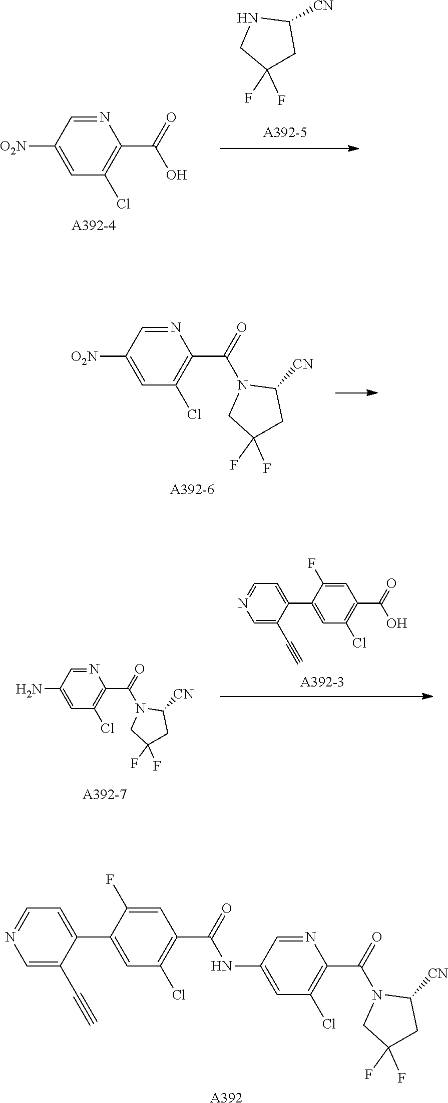

### Step 1: Synthesis of Compound A392-3

A392-1 (253 mg, 1 mmol), A392-2 (161 mg, 1.1 mmol), PdCl₂(dppf)CH₂Cl₂ (10 mg, 0.012 mol), K₂CO₃ (276 mg, 2 mmol), dioxane (5 mL) and H₂O (1 mL) were added to a 50 mL reaction bottle and replaced with nitrogen. The reaction mixture was heated to 90° C. and reacted for 4 hours. The end of the reaction were monitored by LCMS and stopped heating. The reaction solution was diluted with EA (50 mL), washed twice with saturated NaCl solution. The organic phase was concentrated and separated by silica gel column (PE/EA=1/1) to obtain product A392-3 (210 mg, 0.76 mmol), LC-MS (ES⁺): m/z 276[M+H]⁺.

### Step 2: Synthesis of Compound A392-6

A392-4 (202 mg, 1 mmol), A392-5 (158 mg, 1.2 mmol), HATU (570 mg, 1.5 mmol), DIEA (258 mg, 2 mmol), DMF (5 mL) were added to a 50 mL reaction flask, and heated to 80° C. and reacted for 3 hours. Then the reaction solution was stopped heating and cooled to room temperature. The reaction solution was diluted with EA (50 mL), washed twice with saturated NaCl solution. The organic phase was concentrated and separated by silica gel column (PE/EA=1/1) to obtain product A392-6 (187 mg, 0.59 mmol), LC-MS (ES⁺): m/z 317[M+H]⁺

### Step 3: Synthesis of Compound A392-7

A392-6 (158 mg, 2 mmol) and ethanol (5 mL), H₂O (3 ml) were added to a 50 mL reaction flask and stirred. Then Fe powder (280 mg, 5 mmol) and NH₄Cl (265 mg, 5 mmol) was added in batches. After the addition was completed, heating to 70° C. and reacting for 2 hours. Then the reaction solution was stopped heating and cooled to room temperature. The reaction solution was filtered and concentrated. The leftover was separated by silica gel column (DCM/MeOH=20/1) to obtain product A392-7 (103 mg, 0.36 mmol), LC-MS (ES⁺): m/z 287[M+H]⁺

### Step 4: Synthesis of Compound A392

A392-7 (50 mg, 0.17 mmol), A392-3 (48 mg, 0.17 mmol), pyridine (67 mg, 0.85 mmol) and DCM (5 mL) were added to a 50 mL reaction flask, and stirred at 0° C. And POCl₃ (80 mg, 0.52 mmol) was added dropwise. After the dripping, the reaction was stirred at room temperature for 2 hours. The reaction solution was diluted with DCM (50 mL), washed twice with saturated NaCl solution. The organic phase was concentrated and separated by silica gel column (PE/EA=1/1) to obtain product A392 (38 mg, 0.15 mmol), LC-MS (ES⁺): m/z 544[M+H]⁺

### Example 393: Synthesis of Compound A393

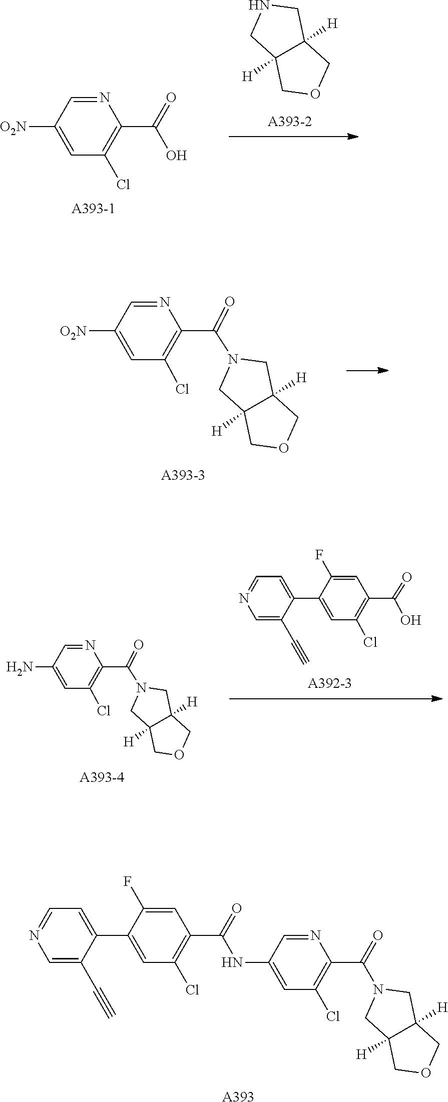

### Step 1: Synthesis of Compound A393-3

A393-1 (202 mg, 1 mmol), A393-2 (124 mg, 1.1 mmol), HATU (570 mg, 1.5 mmol), DIEA (258 mg, 2 mmol) and DMF (5 mL) were added to a 50 mL reaction flask, and heated to 80° C. for 3 hours. Then the reaction solution was stopped heating and cooled to room temperature. The reaction solution was diluted with EA (50 mL), washed twice with saturated NaCl solution. The organic phase was concentrated and separated by silica gel column (PE/EA=1/1) to obtain product A393-3 (170 mg, 0.57 mmol), LC-MS (ES⁺): m/z 298[M+H]⁺

### Step 2: Synthesis of Compound A393-4

A393-3 (150 mg, 0.51 mmol) and ethanol (5 mL), H₂O (3 ml) were added to a 50 mL reaction flask and stirred. Then Fe powder (280 mg, 5 mmol) and NH₄Cl (265 mg, 5 mmol) was added in batches. After the addition was completed, heating to 70° C. and reacting for 2 hours. Then the reaction solution was stopped heating and cooled to room temperature. The reaction solution was filtered- and concentrated. The leftover was separated by silica gel column (DCM/MeOH=20/1) to obtain product A393-4 (105 mg, 0.39 mmol), LC-MS (ES⁺): m/z 268[M+H]⁺

### Step 3: Synthesis of Compound A393

A393-4 (47 mg, 0.18 mmol), A392-3 (48 mg, 0.16 mmol), pyridine (67 mg, 0.85 mmol) and DCM (5 mL) were added to a 50 mL reaction flask, and stirred and cooled to 0° C. Then POCl₃ (78 mg, 0.52 mmol) was added dropwise. After the dripping, the reaction was stirred at room temperature for 2 hours. The reaction solution was diluted with DCM (50 mL), washed twice with saturated NaCl solution. The organic phase was concentrated and separated by silica gel column (PE/EA=1/1) to obtain product A393 (30 mg, 0.057 mmol), LC-MS (ES⁺): m/z 525[M+H]⁺

### Example 395: Synthesis of Compound A395

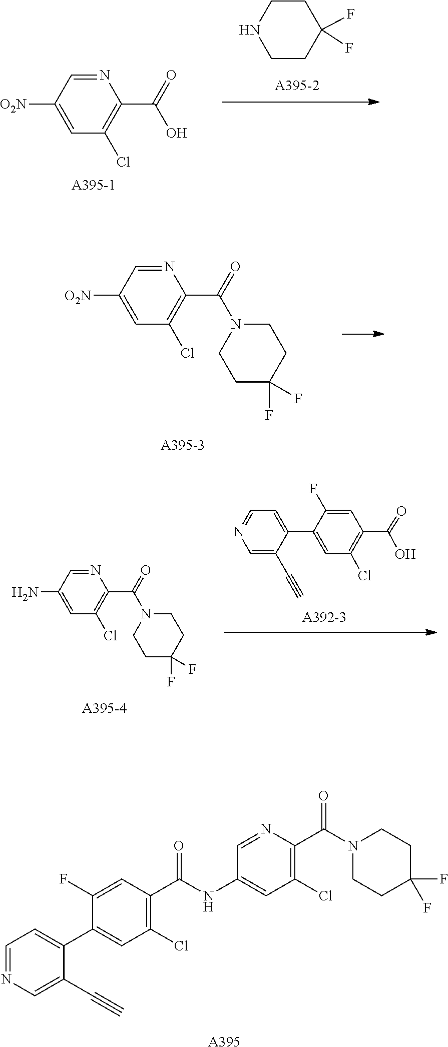

### Step 1: Synthesis of Compound A395-3

A395-1 (202 mg, 1 mmol), A395-2 (133 mg, 1.1 mmol), HATU (570 mg, 1.5 mmol), DIEA (258 mg, 2 mmol) and DMF (5 mL) were added to a 50 mL reaction flask, and heated to 80° C. for 3 hours. Then the reaction solution was stopped heating and cooled to room temperature. The reaction solution was diluted with EA (50 mL), washed twice with saturated NaCl solution. The organic phase was concentrated and separated by silica gel column (PE/EA=1/1) to obtain product A395-3 (150 mg, 0.49 mmol), LC-MS (ES⁺): m/z 306[M+H]⁺

### Step 2: Synthesis of Compound A395-4

A395-3 (150 mg, 0.49 mmol) and ethanol (5 mL), H₂O (3 ml) were added to a 50 mL reaction flask and stirred. Then Fe powder (270 mg, 5 mmol), H₂O (3 ml) was added in batches. After the addition was completed, heating to 70° C. for 2 hours. Then the reaction solution was stopped heating and cooled to room temperature. The reaction solution was filtered and concentrated. The leftover was separated by silica gel column (DCM/MeOH=20/1) to obtain product A395-4 (100 mg, 0.36 mmol), LC-MS (ES⁺): m/z 276[M+H]⁺

### Step 3: Synthesis of Compound A395

A395-4 (48 mg, 0.17 mmol), A392-3 (48 mg, 0.16 mmol), pyridine (67 mg, 0.85 mmol) and DCM (5 mL) were added to a 50 mL reaction flask, and stirred at 0° C. And POCl₃ (78 mg, 0.51 mmol) was added dropwise. After the dripping, the reaction was stirred at room temperature for 2 hours. The reaction solution was diluted with DCM (50 mL), washed twice with saturated NaCl solution. The organic phase was concentrated and separated by silica gel column (PE/EA=1/1) to obtain product A395 (22 mg, 0.041 mmol), LC-MS (ES⁺): m/z 533 [M+H]⁺

### Example 398: Synthesis of Compound A398

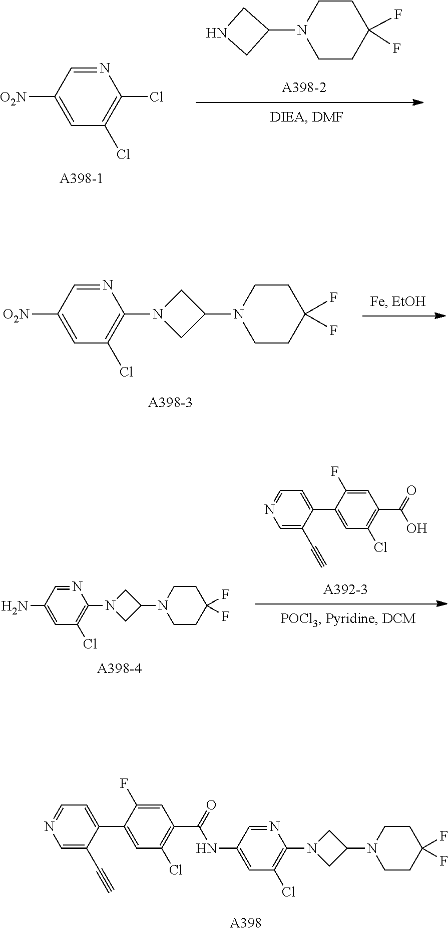

### Step 1: Synthesis of Compound A398-3

Into a 100-mL round-bottom flask, was placed A398-1 (192 mg), A398-2 (193 mg), DIEA (258 mg) and DMF (5 mL), and The resulting mixture was stirred at 100° C. for 3 hours. Then the reaction solution was cooled to room temperature, diluted with EA (50 mL), washed twice with saturated NaCl solution. The organic phase was concentrated and purified by silica gel column (PE/EA=1/1) to give compound A398-3 (220 mg), LC-MS (ES⁺): m/z 333[M+H]⁺

### Step 2: Synthesis of Compound A398-4

To a solution of A398-3 (165 mg) in ethanol (5 mL) was added Fe (270 mg) in batches. The reaction was stirred at 70° C. for 2 hours. The reaction solution was filtered, removing of the solvent and purified by silica gel column (DCM/MeOH=20/1) to give compound A398-4 (120 mg), LC-MS (ES⁺): m/z 303[M+H]⁺

### Step 3: Synthesis of Compound A398

To a solution of A398-4 (52 mg), A392-3 (48 mg) in DCM (5 mL) was added pyridine (67 mg) at room temperature. The mixture was cooled to 0° C. in an ice bath. Then POCl₃ (78 mg) was added dropwise. The reaction was stirred for 1 hour at room temperature and quenched by water, extracted with dichloromethane. The organic layer was washed with brine, dried over anhydrous Na₂SO₄ and concentrated in vacuum. The residue was purified by silica gel column (PE/EA=1/1) to give compound A398 (40 mg), LC-MS (ES⁺): m/z 560 [M+H]⁺

### Example 399: Synthesis of Compound A399

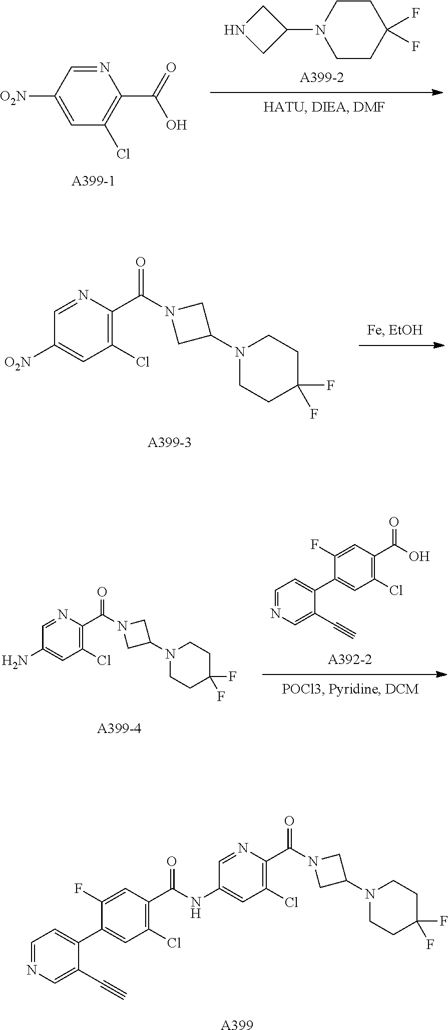

### Step 1: Synthesis of Compound A399-3

To a solution of A399-1 (202 mg) in DMF (5 mL) was added A399-2 (193 mg), HATU (570 mg), DIEA (258 mg). The reaction was stirred at 80° C. for 3 hours. The reaction solution was diluted with EA (50 mL), washed twice with saturated NaCl solution. The organic phase was concentrated and purified by silica gel column (PE/EA=1/1) to give compound A399-3 (136 mg), LC-MS (ES⁺): m/z 361[M+H]⁺

### Step 2: Synthesis of Compound A399-4

To a solution of A399-3 (180 mg) in ethanol (5 mL) was added Fe (270 mg) in batches. The reaction was stirred at 70° C. 2 hours. The reaction solution was filtered, removing of the solvent and purified by silica gel column (DCM/MeOH=20/1) to give compound A399-4 (122 mg), LC-MS (ES⁺): m/z 331[M+H]⁺

### Step 3: Synthesis of Compound A399

To a solution of A399-4 (57 mg) and A392-2 (48 mg) in DCM (5 mL) was added pyridine (67 mg) at room temperature. The mixture was cooled to 0° C. in an ice bath. Then POCl₃ (78 mg) was added dropwise. The reaction was stirred for 2 hour at room temperature and quenched by water, extracted with dichloromethane. The organic layer was washed with brine, dried over anhydrous Na₂SO₄ and concentrated in vacuum. The residue was purified by silica gel column (PE/EA=1/1) to give compound A399 (34 mg), LC-MS (ES⁺): m/z 588 [M+H]⁺

### Example 402: Synthesis of Compound A402

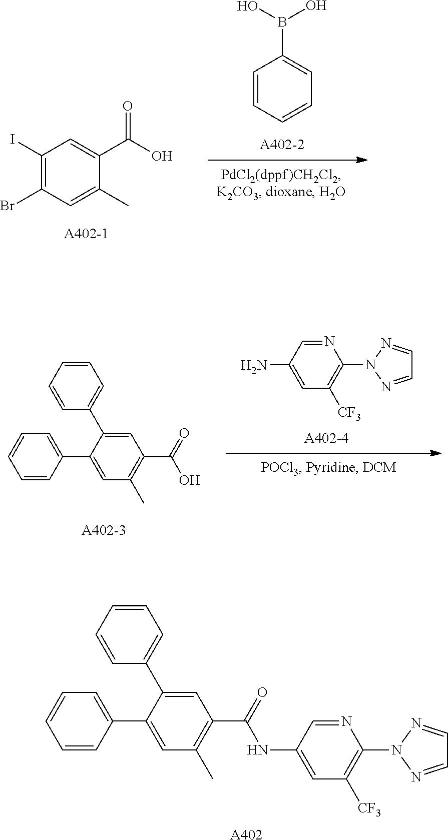 [SMILES: Cc1cc(Br)c(I)cc1C(=O)O ; OB(O)c1ccccc1 ; Cc1cc(-c2ccccc2)c(-c2ccccc2)cc1C(=O)O ; Nc1cnc(-n2nccn2)c(C(F)(F)F)c1 ; Cc1cc(-c2ccccc2)c(-c2ccccc2)cc1C(=O)Nc1cnc(-n2nccn2)c(C(F)(F)F)c1 ; "A402"]

Step 1: Synthesis of Compound A402-3 Into a 100-mL round-bottom flask, was placed A402-1 (340 mg), A402-2 (268 mg), PdCl₂(dppf)CH₂Cl₂ (10 mg), K₂CO₃ (552 mg), dioxane (10 mL) and H₂O (1 mL). The resulting mixture was stirred at 100° C. for 2 hours under nitrogen.

Removing of the solvent and purified by silica gel column (PE/EA=3/1) to give compound A402-3 (230 mg), LC-MS (ES⁺): m/z 289[M+H]⁺.

Step 2: Synthesis of Compound A402 To a solution of A402-3 (50 mg), A402-4 (39 mg), pyridine (67 mg) in DCM (5 mL) was added pyridine (67 mg) at room temperature. The mixture was cooled to 0° C. in an ice bath. Then POCl₃ (78 mg) was added dropwise. The reaction was stirred for 2 hour at room temperature and quenched by water, extracted with dichloromethane.

The organic layer was washed with brine, dried over anhydrous Na₂SO₄ and concentrated in vacuum. The residue was purified by silica gel column (PE/EA=1/1) to give compound A402 (28 mg), LC-MS (ES⁺): m/z 500 [M+H]⁺

### Example 404: Synthesis of Compound A404

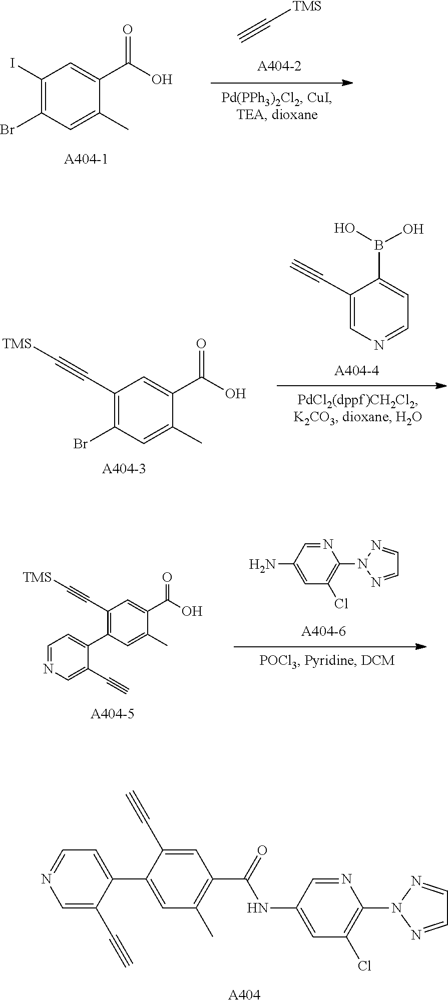

### Step 1: Synthesis of Compound A404-3

Into a 100-mL round-bottom flask, was placed A404-1 (340 mg), A404-2 (98 mg), Pd(PPh₃)₂Cl₂ (10 mg), CuI (10 mg), TEA (552 mg), dioxane (10 mL). The resulting mixture was stirred at 90° C. for 4 hours under nitrogen. The reaction solution was diluted with EA (50 mL), washed twice with saturated NaCl solution. The organic phase was concentrated and purified by silica gel column (PE/EA=1/1) to give compound A404-3 (160 mg), LC-MS (ES⁺): m/z 311[M+H]⁺.

### Step 2: Synthesis of Compound A404-5

Into a 100-mL round-bottom flask, was placed A404-3 (155 mg), A404-4 (110 mg), PdCl₂(dppf)CH₂Cl₂ (10 mg), K₂CO₃ (138 mg), dioxane (5 mL) and H₂O (1 mL). The resulting mixture was stirred at 90° C. for 4 hours under nitrogen. The reaction solution was diluted with EA (50 mL), washed twice with saturated NaCl solution. The organic phase was concentrated and purified by silica gel column (PE/EA=3/1) to give compound A404-5 (110 mg), LC-MS (ES⁺): m/z 334[M+H]⁺.

### Step 3: Synthesis of Compound A404

To a solution of A404-5 (50 mg), A404-6 (29 mg) in DCM (5 mL) was added pyridine (67 mg) at room temperature. The mixture was cooled to 0° C. in an ice bath. Then POCl₃ (78 mg) was added dropwise. The reaction was stirred for 2 hour at room temperature and quenched by water, extracted with dichloromethane. The organic layer was washed with brine, dried over anhydrous Na₂SO₄ and concentrated in vacuum. The residue was purified by silica gel column (PE/EA=1/1) to give compound A404 (17 mg), LC-MS (ES⁺): m/z 439 [M+H]⁺

### Example 405: Synthesis of Compound A405

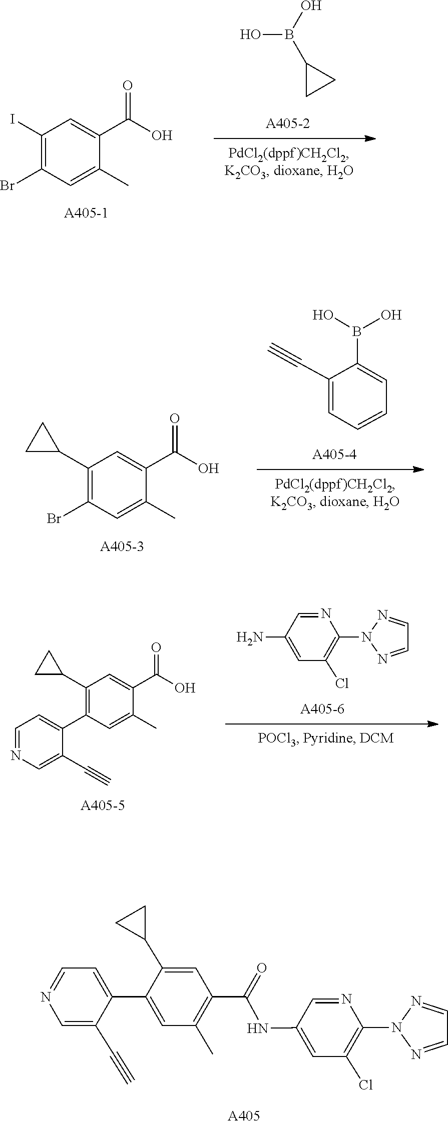

### Step 1: Synthesis of Compound A405-3

Into a 100-mL round-bottom flask, was placed A405-1 (340 mg), A405-2 (86 mg), PdCl₂(dppf)CH₂Cl₂ (10 mg), K₂CO₃ (276 mg), dioxane (5 mL) and H₂O (1 mL). The resulting mixture was stirred at 90° C. for 4 hours under nitrogen. The reaction solution was diluted with EA (50 mL), washed twice with saturated NaCl solution. The organic phase was concentrated and purified by silica gel column (PE/EA=5/1) to give compound A405-3 (110 mg), LC-MS (ES⁺): m/z 254,256[M+H]⁺.

### Step 2: Synthesis of Compound A405-5

Into a 100-mL round-bottom flask, was placed A405-3 (110 mg), A405-4 (95 mg), PdCl₂(dppf)CH₂Cl₂ (10 mg), K₂CO₃ (130 mg), dioxane (5 mL) and H₂O (1 mL). The resulting mixture was stirred at 90° C. for 4 hours under nitrogen. The reaction solution was diluted with EA (50 mL), washed twice with saturated NaCl solution. The organic phase was concentrated and purified by silica gel column (PE/EA=3/1) to give compound A405-5 (60 mg), LC-MS (ES⁺): m/z 278 [M+H]⁺.

### Step 3: Synthesis of Compound A405

To a solution of A405-5 (50 mg), A405-6 (35 mg), pyridine (70 mg) in DCM (5 mL) was added pyridine (70 mg) at room temperature. The mixture was cooled to 0° C. in an ice bath. Then POCl₃ (82 mg) was added dropwise. The reaction was stirred for 2 hour at room temperature and quenched by water, extracted with dichloromethane. The organic layer was washed with brine, dried over anhydrous Na₂SO₄ and concentrated in vacuum. The residue was purified by silica gel column (PE/EA=1/1) to give compound A405 (33 mg), LC-MS (ES⁺): m/z 455 [M+H]⁺.

According to the synthesis method of the above example, select appropriate raw materials and/or marketed example intermediates to synthesize the example compounds in the table below. The raw materials and reagents used are all commercially available.

| | | | MS |
|---|---|---|---|
| Example | Structure | Chemical Name | [M + H]⁺ |
| Example276 (A276) | 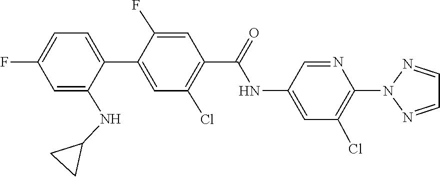 [SMILES: O=C(Nc1cnc(-n2nccn2)c(Cl)c1)c1cc(F)c(-c2ccc(F)cc2NC2CC2)cc1Cl] | 5-chloro-N-(5-chloro-6-(2H- 1,2,3-triazol-2-y1)pyridin-3- yl)-2′-(cyclopropylamino)- 2,4′-difluoro-[1,1′-biphenyl]- 4-carboxamide | 501 |
| Example277 (A277) | 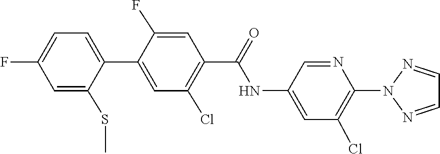 [SMILES: CSc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- yl)-2,4′-difluoro-2′- (methylthio)-[1,1′-bipheny1]- 4-carboxamide | 492 |
| Example278 (A278) | 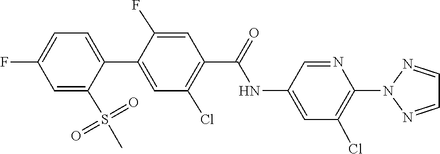 [SMILES: CS(=O)(=O)c1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- y1)-2,4′-difluoro-2′- (methylsulfonyl)-[1,1′- biphenyl]-4-carboxamide | 524 |
| Example279 (A279) | 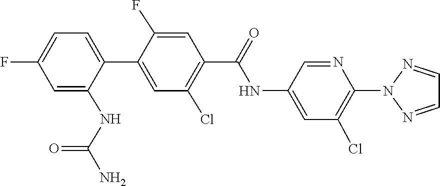 [SMILES: NC(=O)Nc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- yl)-2,4′-difluoro-2′-ureido- [1,1′-biphenyl]-4- carboxamide | 504 |
| Example280 (A280) | 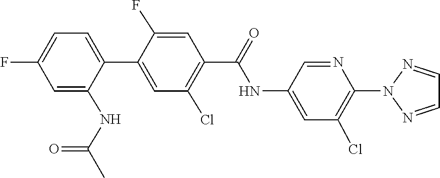 [SMILES: CC(=O)Nc1cc(F)ccc1-c1cc(Cl)c(C(=O)Nc2cnc(-n3nccn3)c(Cl)c2)cc1F] | 2′-acetamido-5-chloro-N-(5- chloro-6-(2H-1,2,3-triazol-2- yl)pyridin-3-y1)-2,4′-difluoro- [1,1′-biphenyl]-4- carboxamide | 503 |
| Example283 (A283) | 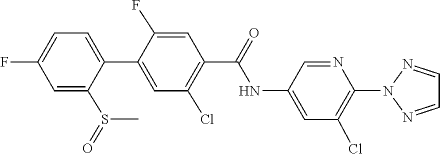 [SMILES: CS(=O)c1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- y1)-2,4′-difluoro-2′- (methylsulfinyl)-[1,1′- biphenyl]-4-carboxamide | 508 |
| Example284 (A284) | 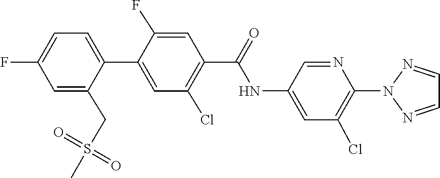 [SMILES: CS(=O)(=O)Cc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- y1)-2,4′-difluoro-2′- ((methylsulfonyl)methyl)- [1,1′-bipheny1]-4- carboxamide | 538 |
| Example285 (A285) | 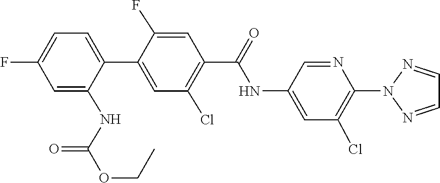 [SMILES: CCOC(=O)Nc1cc(F)ccc1-c1cc(Cl)c(C(=O)Nc2cnc(-n3nccn3)c(Cl)c2)cc1F] | ethyl (5′-chloro-4′-((5-chloro- 6-(2H-1,2,3-triazol-2- yl)pyridin-3-y1)carbamoyl)- 2′,4-difluoro-[1,1′-biphenyl]- 2-yl)carbamate | 533 |
| Example287 (A287) | 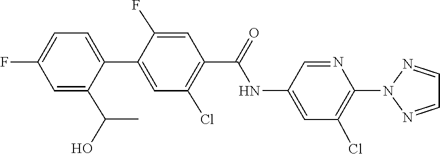 [SMILES: CC(O)c1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- y1)-2,4′-difluoro-2′-vinyl- [1,1′-biphenyl]-4- carboxamide | 490 |
| Example288 (A288) | 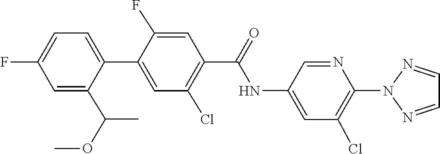 [SMILES: COC(C)c1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- yl)-2,4′-difluoro-2′-(1- hydroxyethyl)-[1,1′- biphenyl]-4-carboxamide | 504 |
| Example289 (A289) | 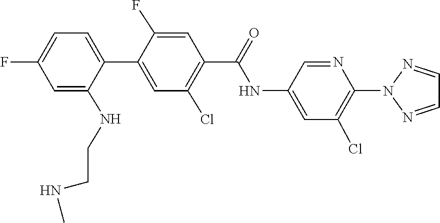 [SMILES: CNCCNc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- yl)-2,4′-difluoro-2′-(1- methoxyethyl)-[1,1′- biphenyl]-4-carboxamide | 518 |
| Example290 (A290) | 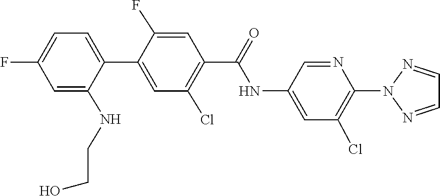 [SMILES: O=C(Nc1cnc(-n2nccn2)c(Cl)c1)c1cc(F)c(-c2ccc(F)cc2NCCO)cc1Cl] | 5-chloro-N-(5-chloro-6-(2H- 1,2,3-triazol-2-y1)pyridin-3- y1)-2,4′-difluoro-2′-((2- (methylamino)ethyl)amino)- [1,1′-biphenyl]-4- carboxamide | 505 |
| Example291 (A291) | 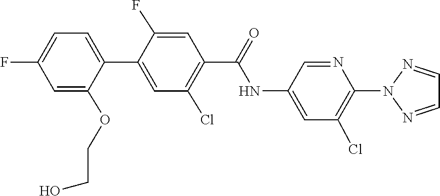 [SMILES: O=C(Nc1cnc(-n2nccn2)c(Cl)c1)c1cc(F)c(-c2ccc(F)cc2OCCO)cc1Cl] | 5-chloro-N-(5-chloro-6-(2H- 1,2,3-triazol-2-yl)pyridin-3- yl)-2,4′-difluoro-2′-(2- hydroxyethoxy)-[1,1′- biphenyl]-4-carboxamide | 506 |
| Example292 (A292) | 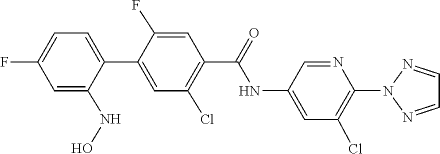 [SMILES: O=C(Nc1cnc(-n2nccn2)c(Cl)c1)c1cc(F)c(-c2ccc(F)cc2NO)cc1Cl] | 5-chloro-N-(5-chloro-6-(2H- 1,2,3-triazol-2-y1)pyridin-3- yl)-2,4′-difluoro-2′- (hydroxyamino)-[1,1′- biphenyl]-4-carboxamide | 477 |
| Example293 (A293) | 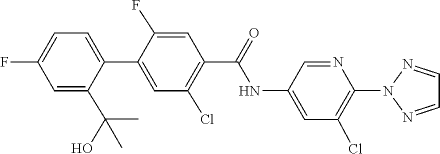 [SMILES: CC(C)(O)c1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- yl)-2,4′-difluoro-2′-(2- hydroxypropan-2-y1)-[1,1′- biphenyl]-4-carboxamide | 504 |
| Example294 (A294) | 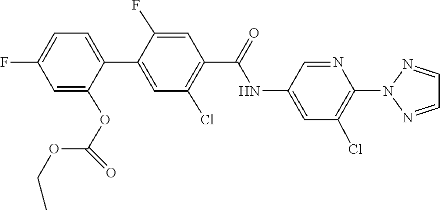 [SMILES: CCOC(=O)Oc1cc(F)ccc1-c1cc(Cl)c(C(=O)Nc2cnc(-n3nccn3)c(Cl)c2)cc1F] | 5′-chloro-4′-((5-chloro-6-(2H- 1,2,3-triazol-2-y1)pyridin-3- yl)carbamoyl)-2′, 4-difluoro- [1,1′-biphenyl]-2-yl ethyl carbonate | 534 |
| Example295 (A295) | 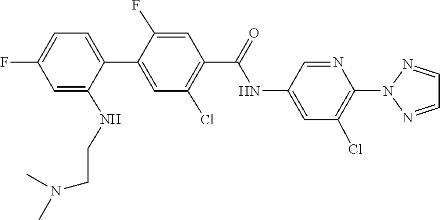 [SMILES: CN(C)CCNc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- y1)-2′-((2- (dimethylamino)ethyl)amino) -2,4′-difluoro-[1,1′-biphenyl]- 4-carboxamide | 532 |
| Example297 (A297) | 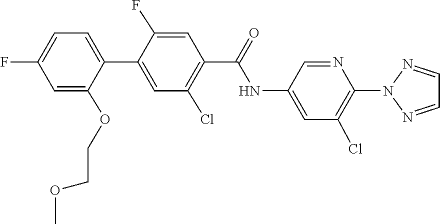 [SMILES: COCCOc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- yl)-2,4′-difluoro-2′-(2- methoxyethoxy)-[1,1′- biphenyl]-4-carboxamide | 520 |
| Example299 (A299) | 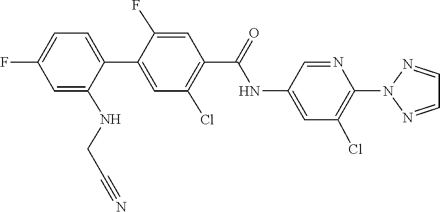 [SMILES: N#CCNc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- yl)-2′-((cyanomethyl)amino)- 2,4′-difluoro-[1,1′-biphenyl]- 4-carboxamide | 500 |
| Example301 (A301) | 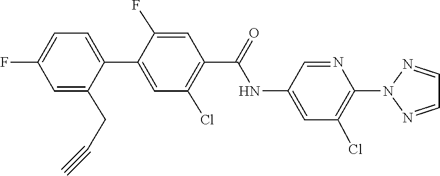 [SMILES: C#CCc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-yl)pyridin-3- y1)-2,4′-difluoro-2′-(prop-2- yn-1-y1)-[1,1′-biphenyl]-4- carboxamide | 484 |
| Example303 (A303) | 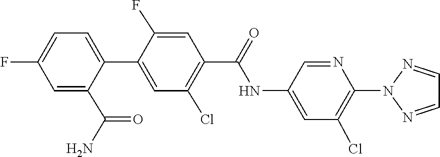 [SMILES: NC(=O)c1cc(F)ccc1-c1cc(Cl)c(C(=O)Nc2cnc(-n3nccn3)c(Cl)c2)cc1F] | 5′-chloro-N4′-(5-chloro-6- (2H-1,2,3-triazol-2- yl)pyridin-3-y1)-2′,4-difluoro- [1,1′-biphenyl]-2,4′- dicarboxamide | 489 |
| Example304 (A304) | 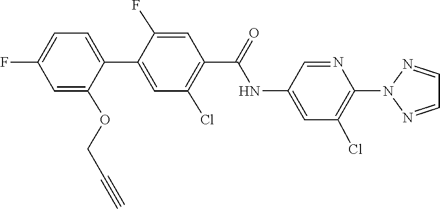 [SMILES: C#CCOc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- y1)-2,4′-difluoro-2′-(prop-2- yn-1-yloxy)-[1,1′-biphenyl]- 4-carboxamide | 500 |
| Example305 (A305) | 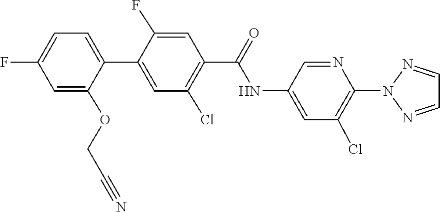 [SMILES: N#CCOc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- yl)-2′-(cyanomethoxy)-2,4′- difluoro-[1,l′-biphenyl]-4- carboxamide | 501 |
| Example306 (A306) | 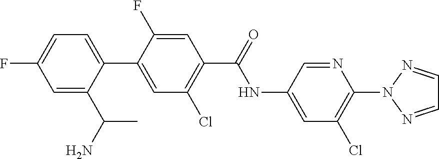 [SMILES: CC(N)c1cc(F)ccc1-c1cc(Cl)c(C(=O)Nc2cnc(-n3nccn3)c(Cl)c2)cc1F] | 2′-(1-aminoethyl)-5-chloro-N- (5-chloro-6-(2H-1,2,3-triazol- 2-y1)pyridin-3-y1)-2,4′- difluoro-[1,1′-biphenyl]-4- carboxamide | 489 |
| Example307 (A307) | 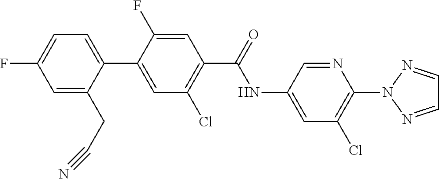 [SMILES: N#CCc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- yl)-2′-(cyanomethyl)-2,4′- difluoro-[1,1′-bipheny1]-4- carboxamide | 485 |
| Example309 (A309) | 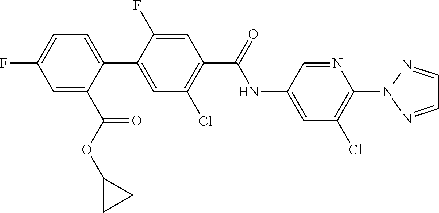 [SMILES: O=C(Nc1cnc(-n2nccn2)c(Cl)c1)c1cc(F)c(-c2ccc(F)cc2C(=O)OC2CC2)cc1Cl] | cyclopropyl 5′-chloro-4′-((5- chloro-6-(2H-1,2,3-triazol-2- yl)pyridin-3-yl)carbamoyl)- 2′,4-difluoro-[1,1′-biphenyl]- 2-carboxylate | 530 |
| Example311 (A311) | 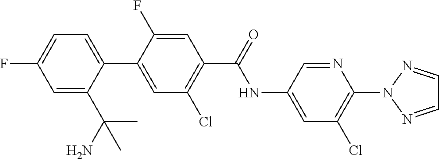 [SMILES: CC(C)(N)c1cc(F)ccc1-c1cc(Cl)c(C(=O)Nc2cnc(-n3nccn3)c(Cl)c2)cc1F] | 2′-(2-aminopropan-2-y1)-5- chloro-N-(5-chloro-6-(2H- 1,2,3-triazol-2-y1)pyridin-3- y1)-2,4′-difluoro-[1,1′- biphenyl]-4-carboxamide | 503 |
| Example312 (A312) | 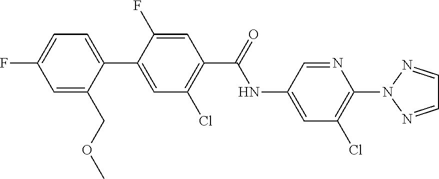 [SMILES: COCc1cc(F)ccc1-c1cc(Cl)c(C(=O)Nc2cnc(-n3nccn3)c(Cl)c2)cc1F] | 5-chloro-N-(5-chloro-6-(2H- 1,2,3-triazol-2-y1)pyridin-3- yl)-2,4′-difluoro-2′- (methoxymethyl)-[1,1′- biphenyl]-4-carboxamide | 490 |
| Example313 (A313) | 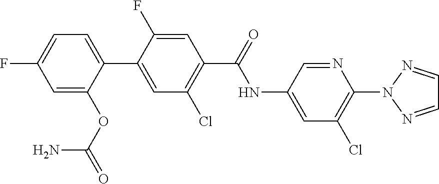 [SMILES: NC(=O)Oc1cc(F)ccc1-c1cc(Cl)c(C(=O)Nc2cnc(-n3nccn3)c(Cl)c2)cc1F] | 5′-chloro-4′-((5-chloro-6-(2H- 1,2,3-triazol-2-y1)pyridin-3- yl)carbamoyl)-2′,4-difluoro- [1,1′-biphenyl]-2-y1 carbamate | 505 |
| Example314 (A314) | 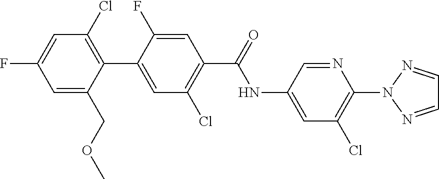 [SMILES: COCc1cc(F)cc(Cl)c1-c1cc(Cl)c(C(=O)Nc2cnc(-n3nccn3)c(Cl)c2)cc1F] | 2′,5-dichloro-N-(5-chloro-6- (2H-1,2,3-triazol-2- yl)pyridin-3-y1)-2,4′-difluoro- 6′-(methoxymethyl)-[1,1′- biphenyl]-4-carboxamide | 525 |
| Example317 (A317) | 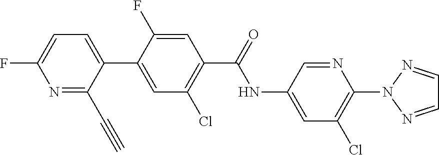 [SMILES: C#Cc1nc(F)ccc1-c1cc(Cl)c(C(=O)Nc2cnc(-n3nccn3)c(Cl)c2)cc1F] | 2-chloro-N-(5-chloro-6-(2H- 1,2,3-triazol-2-y1)pyridin-3- yl)-4-(2-ethyny1-6- fluoropyridin-3-y1)-5- fluorobenzamide | 471 |
| Example320 (A320) | 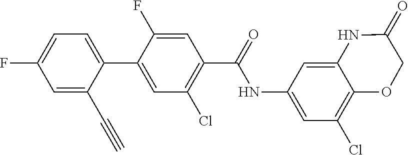 | 5-chloro-N-(8-chloro-3-oxo- 3,4-dihydro-2H- benzo[b][1,4]oxazin-6-yl)-2′- ethyny1-2,4′-difluoro-[1,1′- biphenyl]-4-carboxamide | 473 |
| Example321 (A321) | 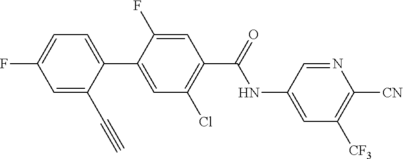 [SMILES: C#Cc1cc(F)ccc1-c1cc(Cl)c(C(=O)Nc2cnc(C#N)c(C(F)(F)F)c2)cc1F] | 5-chloro-N-(6-cyano-5- (trifluoromethyl)pyridin-3- yl)-2′-ethyny1-2,4′-difluoro- [1,1′-bipheny1]-4- carboxamide | 462 |
| Example322 (A322) | 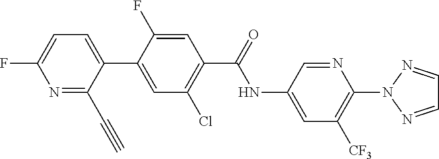 [SMILES: C#Cc1nc(F)ccc1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-2-chloro-4-(2-ethynyl-6- fluoropyridin-3-y1)-5- fluorobenzamide | 505 |
| Example324 (A324) | 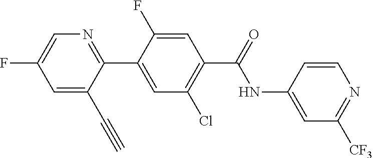 [SMILES: C#Cc1cc(F)cnc1-c1cc(Cl)c(C(=O)Nc2ccnc(C(F)(F)F)c2)cc1F] | 2-chloro-4-(3-ethyny1-5- fluoropyridin-2-y1)-5-fluoro- N-(2- (trifluoromethyl)pyridin-4- yl)benzamide | 438 |
| Example325 (A325) | 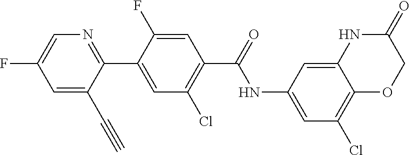 [SMILES: C#Cc1cc(F)cnc1-c1cc(Cl)c(C(=O)Nc2cc(Cl)c3c(c2)NC(=O)CO3)cc1F] | 2-chloro-N-(8-chloro-3-oxo- 3,4-dihydro-2H- benzo[b][1,4]oxazin-6-yl)-4- (3-ethyny1-5-fluoropyridin-2- yl)-5-fluorobenzamide | 474 |
| Example326 (A326) | 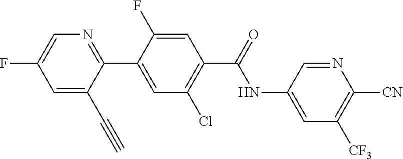 [SMILES: C#Cc1cc(F)cnc1-c1cc(Cl)c(C(=O)Nc2cnc(C#N)c(C(F)(F)F)c2)cc1F] | 2-chloro-N-(6-cyano-5- (trifluoromethyl)pyridin-3- yl)-4-(3-ethyny1-5- fluoropyridin-2-y1)-5- fluorobenzamide | 463 |
| Example328 (A328) | 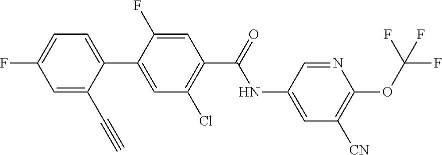 [SMILES: C#Cc1cc(F)ccc1-c1cc(Cl)c(C(=O)Nc2cnc(OC(F)(F)F)c(C#N)c2)cc1F] | 5-chloro-N-(5-cyano-6- (trifluoromethoxy)pyridin-3- y1)-2′-ethynyl-2,4′-difluoro- [1,1′-bipheny1]-4- carboxamide | 478 |
| Example329 (A329) | 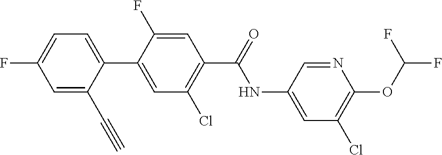 [SMILES: C#Cc1cc(F)ccc1-c1cc(Cl)c(C(=O)Nc2cnc(OC(F)F)c(Cl)c2)cc1F] | 5-chloro-N-(5-chloro-6- (difluoromethoxy)pyridin-3- y1)-2′-ethynyl-2,4′-difluoro- [1,l′-bipheny1]-4- carboxamide | 469 |
| Example330 (A330) | 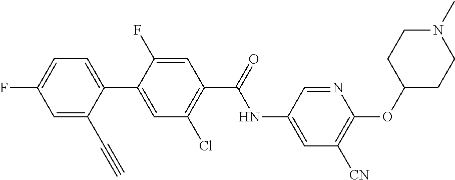 [SMILES: C#Cc1cc(F)ccc1-c1cc(Cl)c(C(=O)Nc2cnc(OC3CCN(C)CC3)c(C#N)c2)cc1F] | 5-chloro-N-(5-cyano-6-((1- methylpiperidin-4- yl)oxy)pyridin-3-y1)-2′- ethyny1-2,4′-difluoro-[1,1′- biphenyl]-4-carboxamide | 507 |
| Example333 (A333) | 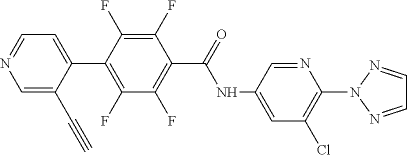 [SMILES: C#Cc1cnccc1-c1c(F)c(F)c(C(=O)Nc2cnc(-n3nccn3)c(Cl)c2)c(F)c1F] | N-(5-chloro-6-(2H-1,2,3- triazol-2-yl)pyridin-3-y1)-4- (3-ethynylpyridin-4-y1)- 2,3,5,6-tetrafluorobenzamide | 473 |
| Example334 (A334) | 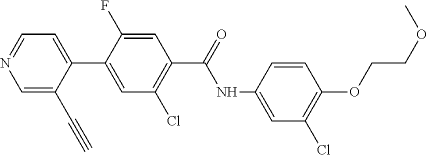 [SMILES: C#Cc1cnccc1-c1cc(Cl)c(C(=O)Nc2ccc(OCCOC)c(Cl)c2)cc1F] | 2-chloro-N-(3-chloro-4-(2- methoxyethoxy)phenyl)-4-(3- ethynylpyridin-4-yl)-5- fluorobenzamide | 459 |
| Example335 (A335) | 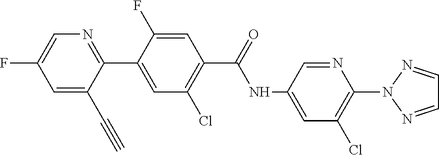 [SMILES: C#Cc1cc(F)cnc1-c1cc(Cl)c(C(=O)Nc2cnc(-n3nccn3)c(Cl)c2)cc1F] | 2-chloro-N-(5-chloro-6-(2H- 1,2,3-triazol-2-yl)pyridin-3- y1)-4-(3-ethyny1-5- fluoropyridin-2-y1)-5- fluorobenzamide | 471 |
| Example336 (A336) | 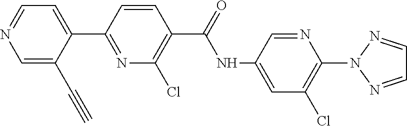 [SMILES: C#Cc1cnccc1-c1ccc(C(=O)Nc2cnc(-n3nccn3)c(Cl)c2)c(Cl)n1] | 6-chloro-N-(5-chloro-6-(2H- 1,2,3-triazol-2-yl)pyridin-3- yl)-3′-ethynyl-[2,4′- bipyridine]-5-carboxamide | 436 |
| Example337 (A337) | 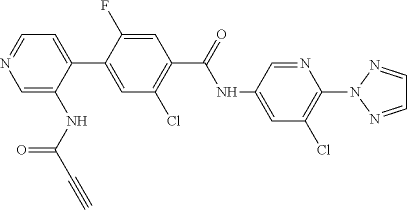 [SMILES: C#CC(=O)Nc1cnccc1-c1cc(Cl)c(C(=O)Nc2cnc(-n3nccn3)c(Cl)c2)cc1F] | 2-chloro-N-(5-chloro-6-(2H- 1,2,3-triazol-2-yl)pyridin-3- y1)-5-fluoro-4-(3- propiolamidopyridin-4- yl)benzamide | 496 |
| Example338 (A338) | 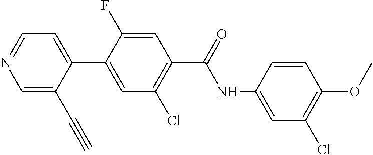 [SMILES: C#Cc1cnccc1-c1cc(Cl)c(C(=O)Nc2ccc(OC)c(Cl)c2)cc1F] | 2-chloro-N-(3-chloro-4- methoxyphenyl)-4-(3- ethynylpyridin-4-y1)-5- fluorobenzamide | 415 |
| Example339 (A339) | 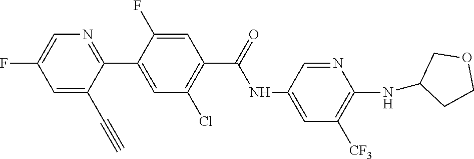 [SMILES: C#Cc1cc(F)cnc1-c1cc(Cl)c(C(=O)Nc2cnc(NC3CCOC3)c(C(F)(F)F)c2)cc1F] | 2-chloro-4-(3-ethyny1-5- fluoropyridin-2-y1)-5-fluoro- N-(6-((tetrahydrofuran-3- yl)amino)-5- (trifluoromethyl)pyridin-3- yl)benzamide | 523 |
| Example340 (A340) | 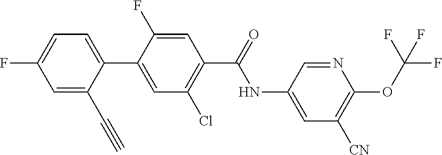 [SMILES: C#Cc1cc(F)ccc1-c1cc(Cl)c(C(=O)Nc2cnc(OC(F)(F)F)c(C#N)c2)cc1F] | N-(5-chloro-6-(2H-1,2,3- triazol-2-yl)pyridin-3-y1)-4- (3-ethyny1-5-fluoropyridin-2- yl)-2,3,5,6- tetrafluorobenzamide | 491 |
| Example341 (A341) | 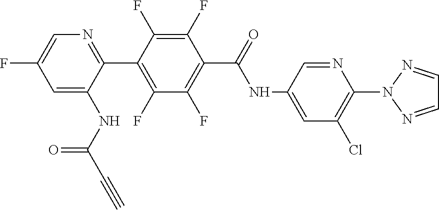 [SMILES: C#CC(=O)Nc1cc(F)cnc1-c1c(F)c(F)c(C(=O)Nc2cnc(-n3nccn3)c(Cl)c2)c(F)c1F] | N-(5-chloro-6-(2H-1,2,3- triazol-2-y1)pyridin-3-y1)- 2,3,5,6-tetrafluoro-4-(5- fluoro-3- propiolamidopyridin-2- yl)benzamide | 534 |
| Example342 (A342) | 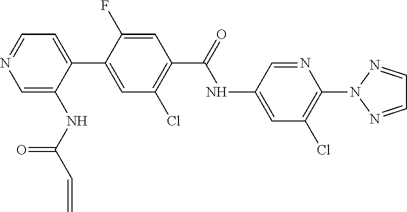 [SMILES: C=CC(=O)Nc1cnccc1-c1cc(Cl)c(C(=O)Nc2cnc(-n3nccn3)c(Cl)c2)cc1F] | 4-(3-acrylamidopyridin-4-y1)- 2-chloro-N-(5-chloro-6-(2H- 1,2,3-triazol-2-y1)pyridin-3- y1)-5-fluorobenzamide | 498 |
| Example345 (A345) | 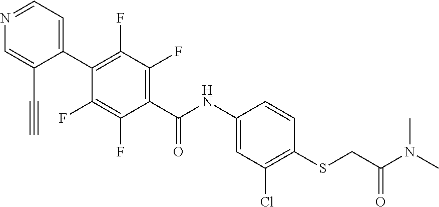 [SMILES: C#Cc1cnccc1-c1c(F)c(F)c(C(=O)Nc2ccc(SCC(=O)N(C)C)c(Cl)c2)c(F)c1F] | N-(3-chloro-4-((2- (dimethylamino)-2- oxoethyl)thio)phenyl)-4-(3- ethynylpyridin-4-yl)-2,3,5,6- tetrafluorobenzamide | 522 |
| Example346 (A346) | 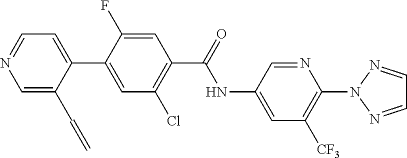 [SMILES: C=Cc1cnccc1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-2-chloro-5-fluoro-4-(3- vinylpyridin-4-y1)benzamide | 489 |
| Example347 (A347) | 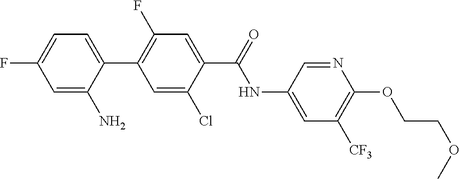 [SMILES: COCCOc1ncc(NC(=O)c2cc(F)c(-c3ccc(F)cc3N)cc2Cl)cc1C(F)(F)F] | 2′-amino-5-chloro-2,4′- difluoro-N-(6-(2- methoxyethoxy)-5- (trifluoromethyl)pyridin-3- yl)-[1,1′-bipheny1]-4- carboxamide | 502 |
| Example348 (A348) | 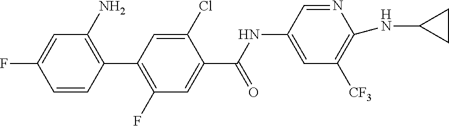 [SMILES: Nc1cc(F)ccc1-c1cc(Cl)c(C(=O)Nc2cnc(NC3CC3)c(C(F)(F)F)c2)cc1F] | 2′-amino-5-chloro-N-(6- (cyclopropylamino)-5- (trifluoromethyl)pyridin-3- y1)-2,4′-difluoro-[1,1′- biphenyl]-4-carboxamide | 483 |
| Example349 (A349) | 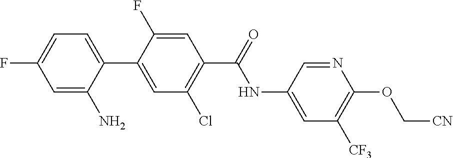 [SMILES: N#CCOc1ncc(NC(=O)c2cc(F)c(-c3ccc(F)cc3N)cc2Cl)cc1C(F)(F)F] | 2′-amino-5-chloro-N-(6- (cyanomethoxy)-5- (trifluoromethyl)pyridin-3- y1)-2,4′-difluoro-[1,1′- bipheny1]-4-carboxamide | 483 |
| Example350 (A350) | 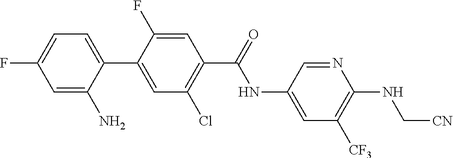 [SMILES: N#CCNc1ncc(NC(=O)c2cc(F)c(-c3ccc(F)cc3N)cc2Cl)cc1C(F)(F)F] | 2′-amino-5-chloro-N-(6- ((cyanomethyl)amino)-5- (trifluoromethyl)pyridin-3- y1)-2,4′-difluoro-[1,1′- biphenyl]-4-carboxamide | 482 |
| Example351 (A351) | 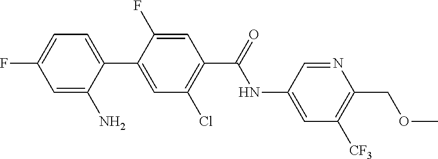 [SMILES: COCc1ncc(NC(=O)c2cc(F)c(-c3ccc(F)cc3N)cc2Cl)cc1C(F)(F)F] | 2′-amino-5-chloro-2,4′- difluoro-N-(6- (methoxymethyl)-5- (trifluoromethyl)pyridin-3- y1)-[1,1′-bipheny1]-4- carboxamide | 472 |
| Example352 (A352) | 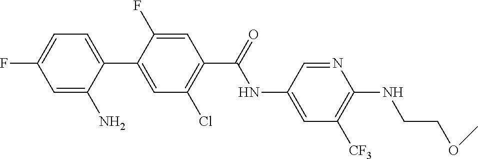 [SMILES: COCCNc1ncc(NC(=O)c2cc(F)c(-c3ccc(F)cc3N)cc2Cl)cc1C(F)(F)F] | 2′-amino-5-chloro-2,4′- difluoro-N-(6-((2- methoxyethyl)amino)-5- (trifluoromethyl)pyridin-3- y1)-[1,1′-biphenyl]-4- carboxamide | 501 |
| Example353 (A353) | 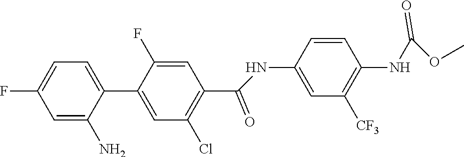 [SMILES: COC(=O)Nc1ccc(NC(=O)c2cc(F)c(-c3ccc(F)cc3N)cc2Cl)cc1C(F)(F)F] | methyl (4-(2′-amino-5-chloro- 2,4′-difluoro-[1,1′-bipheny1]- 4-carboxamido)-2- (trifluoromethyl)phenyl) carbamate | 500 |
| Example354 (A354) | 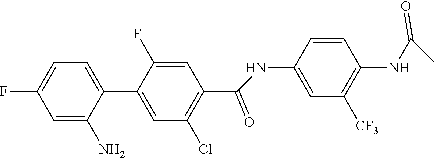 [SMILES: CC(=O)Nc1ccc(NC(=O)c2cc(F)c(-c3ccc(F)cc3N)cc2Cl)cc1C(F)(F)F] | N-(4-acetamido-3- (trifluoromethyl)phenyl)-2′- amino-5-chloro-2,4′-difluoro- [1,1′-bipheny1]-4- carboxamide | 484 |
| Example355 (A355) | 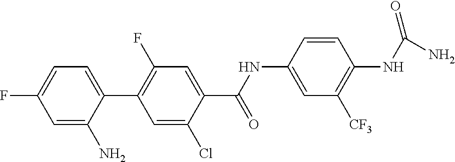 [SMILES: NC(=O)Nc1ccc(NC(=O)c2cc(F)c(-c3ccc(F)cc3N)cc2Cl)cc1C(F)(F)F] | 2′-amino-5-chloro-2,4′- difluoro-N-(3- (trifluoromethyl)-4- ureidophenyl)-[1,1′- biphenyl]-4-carboxamide | 485 |
| Example356 (A356) | 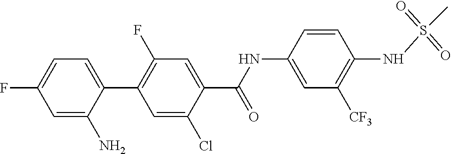 [SMILES: CS(=O)(=O)Nc1ccc(NC(=O)c2cc(F)c(-c3ccc(F)cc3N)cc2Cl)cc1C(F)(F)F] | 2′-amino-5-chloro-2,4′- difluoro-N-(4- (methylsulfonamido)-3- (trifluoromethyl)phenyl)- [1,1′-bipheny1]-4- carboxamide | 520 |
| Example357 (A357) | 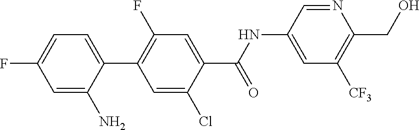 [SMILES: Nc1cc(F)ccc1-c1cc(Cl)c(C(=O)Nc2cnc(CO)c(C(F)(F)F)c2)cc1F] | 2′-amino-5-chloro-2,4′- difluoro-N-(6- (hydroxymethyl)-5- (trifluoromethyl)pyridin-3- yl)-[1,l′-bipheny1]-4- carboxamide | 458 |
| Example358 (A358) | 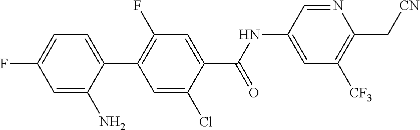 [SMILES: N#CCc1ncc(NC(=O)c2cc(F)c(-c3ccc(F)cc3N)cc2Cl)cc1C(F)(F)F] | 2′-amino-5-chloro-N-(6- (cyanomethyl)-5- (trifluoromethyl)pyridin-3- y1)-2,4′-difluoro-[1,1′- biphenyl]-4-carboxamide | 467 |
| Example359 (A359) | 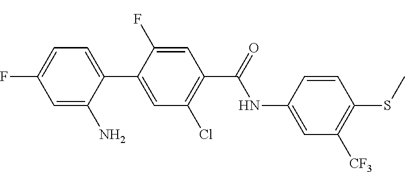 | 2′-amino-5-chloro-2,4′- difluoro-N-(4-(methylthio)-3- (trifluoromethyl)phenyl)- [1,1′-biphenyl]-4- carboxamide | 473 |
| Example360 (A360) | 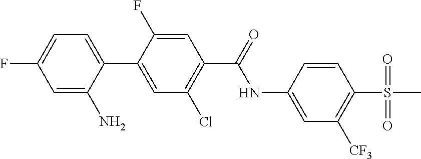 [SMILES: CS(=O)(=O)c1ccc(NC(=O)c2cc(F)c(-c3ccc(F)cc3N)cc2Cl)cc1C(F)(F)F] | 2′-amino-5-chloro-2,4′- difluoro-N-(4- (methylsulfonyl)-3- (trifluoromethyl)phenyl)- [1,1′-bipheny1]-4- carboxamide | 505 |
| Example362 (A362) | 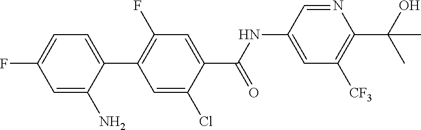 [SMILES: CC(C)(O)c1ncc(NC(=O)c2cc(F)c(-c3ccc(F)cc3N)cc2Cl)cc1C(F)(F)F] | 2′-amino-5-chloro-2,4′- difluoro-N-(6-(2- hydroxypropan-2-y1)-5- (trifluoromethyl)pyridin-3- y1)-[1,1′-bipheny1]-4- carboxamide | 486 |
| Example363 (A363) | 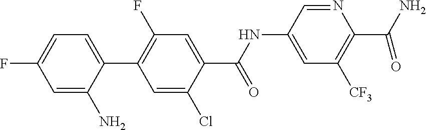 [SMILES: NC(=O)c1ncc(NC(=O)c2cc(F)c(-c3ccc(F)cc3N)cc2Cl)cc1C(F)(F)F] | 5-(2′-amino-5-chloro-2,4′- difluoro-[1,1′-biphenyl]-4- carboxamido)-3- (trifluoromethyl)picolinamide | 471 |
| Example364 (A364) | 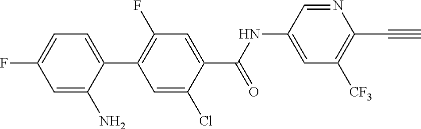 [SMILES: C#Cc1ncc(NC(=O)c2cc(F)c(-c3ccc(F)cc3N)cc2Cl)cc1C(F)(F)F] | 2′-amino-5-chloro-N-(6- ethyny1-5- (trifluoromethyl)pyridin-3- yl)-2,4′-difluoro-[1,1′- biphenyl]-4-carboxamide | 452 |
| Example365 (A365) | 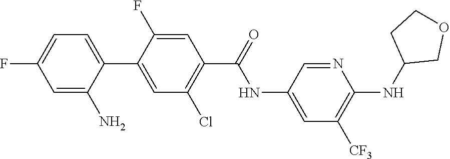 [SMILES: Nc1cc(F)ccc1-c1cc(Cl)c(C(=O)Nc2cnc(NC3CCOC3)c(C(F)(F)F)c2)cc1F] | 2′-amino-5-chloro-2,4′- difluoro-N-(6- ((tetrahydrofuran-3- yl)amino)-5- (trifluoromethyl)pyridin-3- y1)-[1,1′-bipheny1]-4- carboxamide | 513 |
| Example366 (A366) | 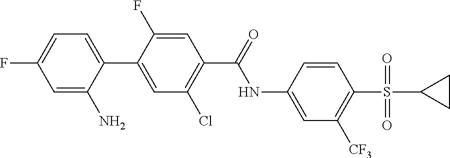 [SMILES: Nc1cc(F)ccc1-c1cc(Cl)c(C(=O)Nc2ccc(S(=O)(=O)C3CC3)c(C(F)(F)F)c2)cc1F] | 2′-amino-5-chloro-N-(4- (cyclopropylsulfony1)-3- (trifluoromethyl)phenyl)-2,4′- difluoro-[1,1′-bipheny1]-4- carboxamide | 531 |
| Example367 (A367) | 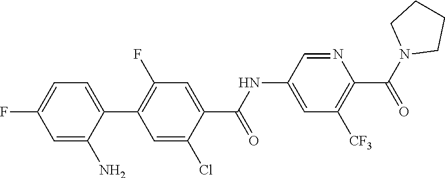 [SMILES: Nc1cc(F)ccc1-c1cc(Cl)c(C(=O)Nc2cnc(C(=O)N3CCCC3)c(C(F)(F)F)c2)cc1F] | 2′-amino-5-chloro-2,4′- difluoro-N-(6-(pyrrolidine-1- carbonyl)-5- (trifluoromethyl)pyridin-3- yl)-[1,l′-biphenyl]-4- carboxamide | 525 |
| Example368 (A368) | 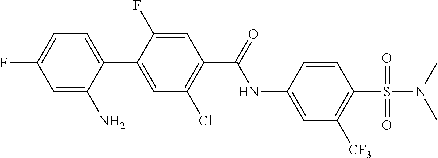 | 2′-amino-5-chloro-N-(4-(N,N- dimethylsulfamoyl)-3- (trifluoromethyl)phenyl)-2,4′- difluoro-[1,1′-bipheny1]-4- carboxamide | 534 |
| Example369 (A369) | 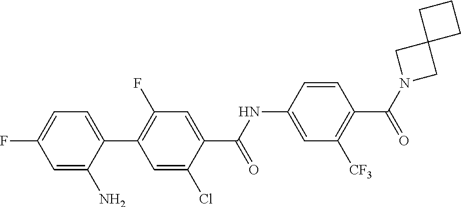 [SMILES: Nc1cc(F)ccc1-c1cc(Cl)c(C(=O)Nc2ccc(C(=O)N3CC4(CCC4)C3)c(C(F)(F)F)c2)cc1F] | N-(4-(2-azaspiro[3.3]heptane- 2-carbony1)-3- (trifluoromethyl)phenyl)-2′- amino-5-chloro-2,4′-difluoro- [1,1′-bipheny1]-4- carboxamide | 550 |
| Example370 (A370) | 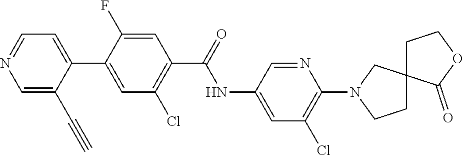 [SMILES: C#Cc1cnccc1-c1cc(Cl)c(C(=O)Nc2cnc(N3CCC4(CCOC4=O)C3)c(Cl)c2)cc1F] | 2-chloro-N-(5-chloro-6-(1- oxo-2-oxa-7- azaspiro[4.4]nonan-7- yl)pyridin-3-y1)-4-(3- ethynylpyridin-4-y1)-5- fluorobenzamide | 525 |
| Example372 (A372) | 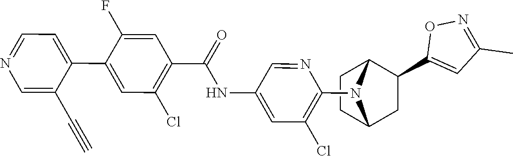 [SMILES: C#Cc1cnccc1-c1cc(Cl)c(C(=O)Nc2cnc(N3[C@H]4CC[C@@H]3[C@@H](c3cc(C)no3)C4)c(Cl)c2)cc1F] | 2-chloro-N-(5-chloro-6- ((1R,2S,4S)-2-(3- methylisoxazol-5-y1)-7- azabicyclo[2.2.1]heptan-7- y1)pyridin-3-y1)-4-(3- ethynylpyridin-4-y1)-5- fluorobenzamide | 562 |
| Example375 (A375) | 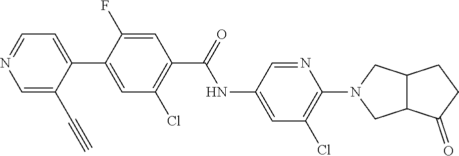 [SMILES: C#Cc1cnccc1-c1cc(Cl)c(C(=O)Nc2cnc(N3CC4CCC(=O)C4C3)c(Cl)c2)cc1F] | 2-chloro-N-(5-chloro-6-(4- oxohexahydrocyclopenta[c]py rrol-2(1H)-yl)pyridin-3-yl)-4- (3-ethynylpyridin-4-y1)-5- fluorobenzamide | 509 |
| Example376 (A376) | 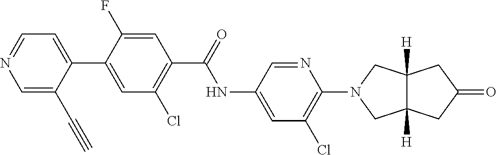 [SMILES: C#Cc1cnccc1-c1cc(Cl)c(C(=O)Nc2cnc(N3C[C@H]4CC(=O)C[C@H]4C3)c(Cl)c2)cc1F] | 2-chloro-N-(5-chloro-6- ((3aR,6aS)-5- oxohexahydrocyclopenta[c]py rrol-2(1H)-yl)pyridin-3-y1)-4- (3-ethynylpyridin-4-y1)-5- fluorobenzamide | 509 |
| Example378 (A378) | 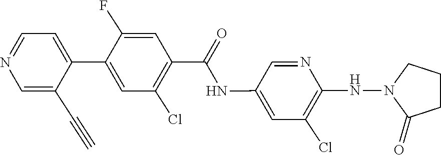 [SMILES: C#Cc1cnccc1-c1cc(Cl)c(C(=O)Nc2cnc(NN3CCCC3=O)c(Cl)c2)cc1F] | 2-chloro-N-(5-chloro-6-((2- oxopyrrolidin-1- yl)amino)pyridin-3-y1)-4-(3- ethynylpyridin-4-y1)-5- fluorobenzamide | 484 |
| Example379 (A379) | 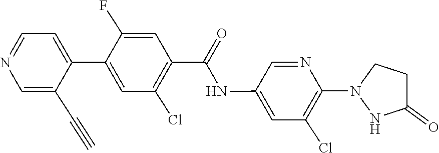 [SMILES: C#Cc1cnccc1-c1cc(Cl)c(C(=O)Nc2cnc(N3CCC(=O)N3)c(Cl)c2)cc1F] | 2-chloro-N-(5-chloro-6-(3- oxopyrazolidin-1-y1)pyridin- 3-y1)-4-(3-ethynylpyridin-4- y1)-5-fluorobenzamide | 470 |
| Example380 (A380) | 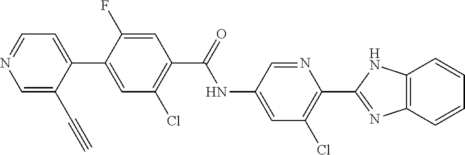 [SMILES: C#Cc1cnccc1-c1cc(Cl)c(C(=O)Nc2cnc(-c3nc4ccccc4[nH]3)c(Cl)c2)cc1F] | N-(6-(1H-benzo[d]imidazol- 2-y1)-5-chloropyridin-3-y1)-2- chloro-4-(3-ethynylpyridin-4- y1)-5-fluorobenzamide | 502 |
| Example382 (A382) | 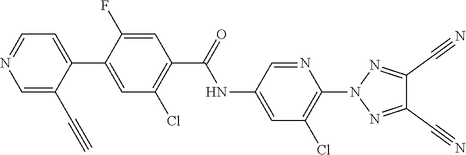 [SMILES: C#Cc1cnccc1-c1cc(Cl)c(C(=O)Nc2cnc(-n3nc(C#N)c(C#N)n3)c(Cl)c2)cc1F] | 2-chloro-N-(5-chloro-6-(4,5- dicyano-2H-1,2,3-triazol-2- y1)pyridin-3-y1)-4-(3- ethynylpyridin-4-y1)-5- fluorobenzamide | 503 |
| Example384 (A384) | 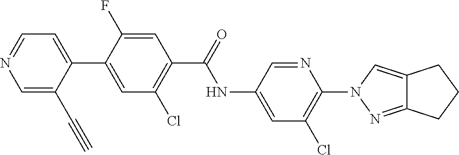 [SMILES: C#Cc1cnccc1-c1cc(Cl)c(C(=O)Nc2cnc(-n3cc4c(n3)CCC4)c(Cl)c2)cc1F] | 2-chloro-N-(5-chloro-6-(5,6- dihydrocyclopenta[c]pyrazol- 2(4H)-y1)pyridin-3-y1)-4-(3- ethynylpyridin-4-y1)-5- fluorobenzamide | 492 |
| Example385 (A385) | 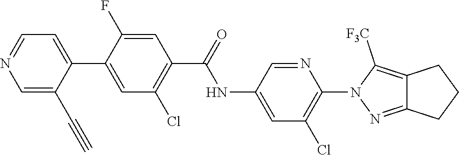 [SMILES: C#Cc1cnccc1-c1cc(Cl)c(C(=O)Nc2cnc(-n3nc4c(c3C(F)(F)F)CCC4)c(Cl)c2)cc1F] | 2-chloro-N-(5-chloro-6-(3- (trifluoromethyl)-5,6- dihydrocyclopenta[c]pyrazol- 2(4H)-yl)pyridin-3-y1)-4-(3- ethynylpyridin-4-y1)-5- fluorobenzamide | 560 |
| Example386 (A386) | 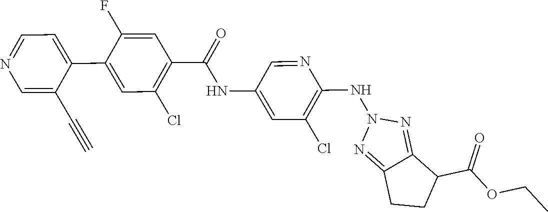 [SMILES: C#Cc1cnccc1-c1cc(Cl)c(C(=O)Nc2cnc(Nn3nc4c(n3)C(C(=O)OCC)CC4)c(Cl)c2)cc1F] | ethyl 2-((3-chloro-5-(2- chloro-4-(3-ethynylpyridin-4- y1)-5- fluorobenzamido)pyridin-2- y1)amino)-2,4,5,6- tetrahydrocyclopenta[d][1,2,3] triazole-4-carboxylate | 580 |
| Example387 (A387) | 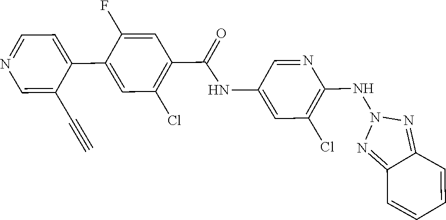 [SMILES: C#Cc1cnccc1-c1cc(Cl)c(C(=O)Nc2cnc(Nn3nc4ccccc4n3)c(Cl)c2)cc1F] | N-(6-((2H- benzo[d][1,2,3]triazol-2- yl)amino)-5-chloropyridin-3- yl)-2-chloro-4-(3- ethynylpyridin-4-y1)-5- fluorobenzamide | 518 |
| Example389 (A389) | 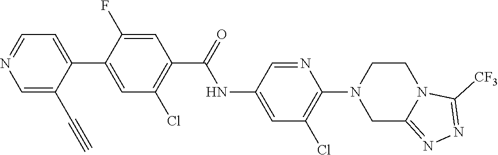 [SMILES: C#Cc1cnccc1-c1cc(Cl)c(C(=O)Nc2cnc(N3CCn4c(nnc4C(F)(F)F)C3)c(Cl)c2)cc1F] | 2-chloro-N-(5-chloro-6-(3- (trifluoromethyl)-5,6-dihydro- [1,2,4]triazolo[4,3-a]pyrazin- 7(8H)-y1)pyridin-3-y1)-4-(3- ethynylpyridin-4-y1)-5- fluorobenzamide | 576 |
| Example390 (A390) | 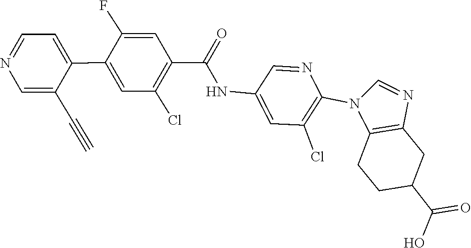 [SMILES: C#Cc1cnccc1-c1cc(Cl)c(C(=O)Nc2cnc(-n3cnc4c3CCC(C(=O)O)C4)c(Cl)c2)cc1F] | 1-(3-chloro-5-(2-chloro-4-(3- ethynylpyridin-4-y1)-5- fluorobenzamido)pyridin-2- yl)-4,5,6,7-tetrahydro-1H- benzo[d]imidazole-5- carboxylic acid | 550 |
| Example391 (A391) | 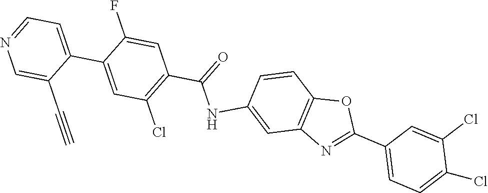 [SMILES: C#Cc1cnccc1-c1cc(Cl)c(C(=O)Nc2ccc3oc(-c4ccc(Cl)c(Cl)c4)nc3c2)cc1F] | 2-chloro-N-(2-(3,4- dichlorophenyl)benzo[d]oxaz ol-5-yl)-4-(3-ethynylpyridin- 4-y1)-5-fluorobenzamide | 536 |
| Example394 (A394) | 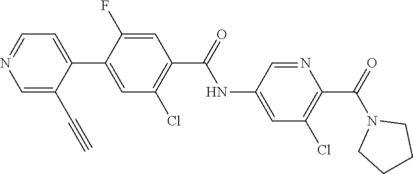 [SMILES: C#Cc1cnccc1-c1cc(Cl)c(C(=O)Nc2cnc(C(=O)N3CCCC3)c(Cl)c2)cc1F] | 2-chloro-N-(5-chloro-6- (pyrrolidine-1- carbonyl)pyridin-3-yl)-4-(3- ethynylpyridin-4-y1)-5- fluorobenzamide | 483 |
| Example396 (A396) | 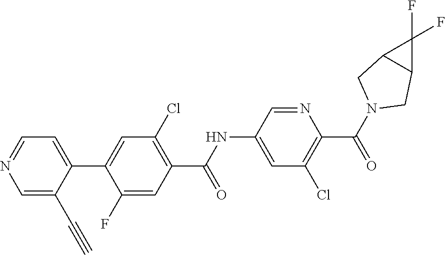 [SMILES: C#Cc1cnccc1-c1cc(Cl)c(C(=O)Nc2cnc(C(=O)N3CC4C(C3)C4(F)F)c(Cl)c2)cc1F] | 2-chloro-N-(5-chloro-6-(6,6- difluoro-3- azabicyclo[3.1.0]hexane-3- carbonyl)pyridin-3-yl)-4-(3- ethynylpyridin-4-y1)-5- fluorobenzamide | 531 |
| Example397 (A397) | 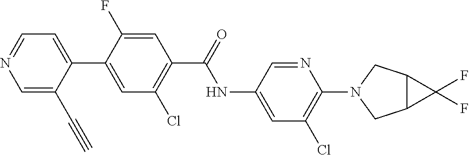 [SMILES: C#Cc1cnccc1-c1cc(Cl)c(C(=O)Nc2cnc(N3CC4C(C3)C4(F)F)c(Cl)c2)cc1F] | 2-chloro-N-(5-chloro-6-(6,6- difluoro-3- azabicyclo[3.1.0]hexan-3- yl)pyridin-3-y1)-4-(3- ethynylpyridin-4-yl)-5- fluorobenzamide | 503 |
| Example400 (A400) | 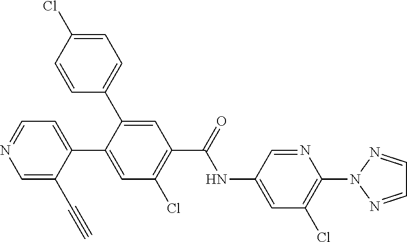 [SMILES: C#Cc1cnccc1-c1cc(Cl)c(C(=O)Nc2cnc(-n3nccn3)c(Cl)c2)cc1-c1ccc(Cl)cc1] | 4,4′-dichloro-N-(5-chloro-6- (2H-1,2,3-triazol-2- y1)pyridin-3-y1)-6-(3- ethynylpyridin-4-yl)-[1,1′- biphenyl]-3-carboxamide | 545 |
| Example401 (A401) | 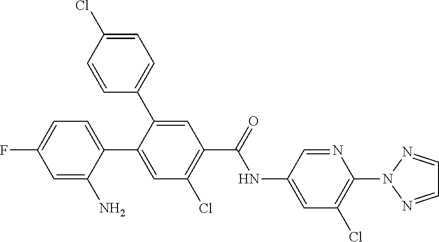 [SMILES: Nc1cc(F)ccc1-c1cc(Cl)c(C(=O)Nc2cnc(-n3nccn3)c(Cl)c2)cc1-c1ccc(Cl)cc1] | 2-amino-4″,5′-dichloro-N-(5- chloro-6-(2H-1,2,3-triazol-2- yl)pyridin-3-y1)-4-fluoro- [1,1′:2′,1″-terphenyl]-4′- carboxamide | 553 |
| Example403 (A403) | 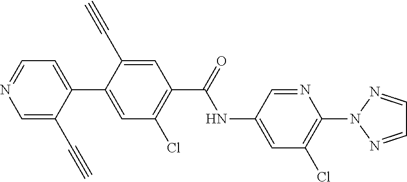 [SMILES: C#Cc1cnccc1-c1cc(Cl)c(C(=O)Nc2cnc(-n3nccn3)c(Cl)c2)cc1C#C] | 2-chloro-N-(5-chloro-6-(2H- 1,2,3-triazol-2-y1)pyridin-3- y1)-5-ethyny1-4-(3- ethynylpyridin-4- yl)benzamide | 459 |
| Example406 (A406) | 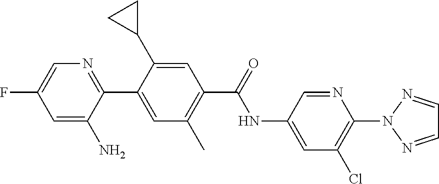 [SMILES: Cc1cc(-c2ncc(F)cc2N)c(C2CC2)cc1C(=O)Nc1cnc(-n2nccn2)c(Cl)c1] | 4-(3-amino-5-fluoropyridin-2- y1)-N-(5-chloro-6-(2H-1,2,3- triazol-2-yl)pyridin-3-y1)-5- cyclopropyl-2- methylbenzamide | 464 |
| Example407 (A407) | 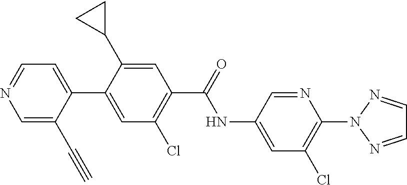 [SMILES: C#Cc1cnccc1-c1cc(Cl)c(C(=O)Nc2cnc(-n3nccn3)c(Cl)c2)cc1C1CC1] | 2-chloro-N-(5-chloro-6-(2H- 1,2,3-triazol-2-y1)pyridin-3- yl)-5-cyclopropyl-4-(3- ethynylpyridin-4- yl)benzamide | 475 |
| Example408 (A408) | 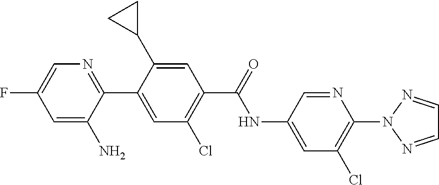 [SMILES: Nc1cc(F)cnc1-c1cc(Cl)c(C(=O)Nc2cnc(-n3nccn3)c(Cl)c2)cc1C1CC1] | 4-(3-amino-5-fluoropyridin-2- yl)-2-chloro-N-(5-chloro-6- (2H-1,2,3-triazol-2- yl)pyridin-3-y1)-5- cyclopropylbenzamide | 484 |

### Example 409: Synthesis of Compound A409

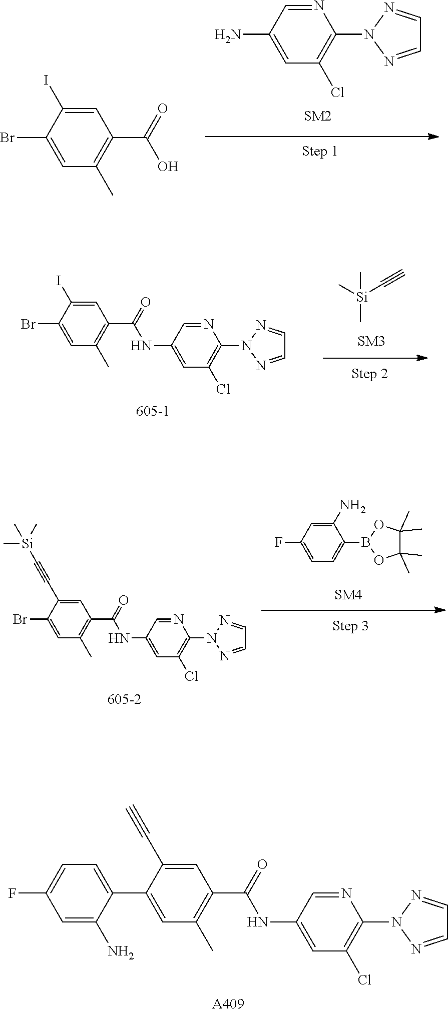

### Step 1: Synthesis of Compound A605-1

Under an ice-water bath, POCl₃ (0.31 g, 2.0 mmoL) was added dropwise to a DCM (10 mL) solution containing SM1 (0.34 g, 1.0 mmoL), SM2 (0.20 g, 1.0 mmoL) and pyridine (0.84 g, 10 mmoL). The reaction mixture was stirred at room temperature for 2 hours. After the reaction was completed, the reaction solution was diluted with DCM (50 mL), washed with saturated brine (3*50 mL). The organic phase was dried over anhydrous sodium sulfate, concentrated and separated using a Flash silica gel column (PE/EA=2/1) to obtain product 605-1 (0.46 g, 0.89 mmoL) as yellow solid. LC-MS (ES⁺): m/z 517[M+H]⁺

### Step 2: Synthesis of Compound A605-2

SM3 (0.1 g, 1.0 mmoL), 605-1 (0.26 g, 0.5 mmoL), CuI (2.0 mg, 0.01 mmoL), Pd(PPh₃)₂Cl₂ (3.5 mg, 0.005 mmoL) and TEA (0.3 g, 3.0 mmoL) were added in sequence to a single-neck bottle containing THF (10 mL) and a stirrer. The reaction liquid container were replaced with nitrogen. The reaction mixture was stirred at room temperature for 5 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product 605-2 (0.21 g, 0.4 mmoL) as white solid. LC-MS (ES⁺): m/z 488 [M+H]⁺

### Step 3: Synthesis of Compound A409

SM4 (0.14 g, 0.6 mmoL), 605-2 (0.21 g, 0.4 mmoL), Pd(dppf)Cl₂ (14.6 mg, 0.02 mmoL) and K₂CO₃ (0.12 g, 0.8 mmoL) were added in sequence to a single-neck bottle containing dioxane (4 mL), H₂O (1 mL) and a stirrer. The reaction liquid container were replaced with nitrogen. The reaction mixture was stirred in a 90° C. oil bath for 8 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product A409 (0.11 g, 0.24 mmoL) as white solid. LC-MS (ES⁺): m/z 447 [M+H]⁺

### Example 412: Synthesis of Compound A412

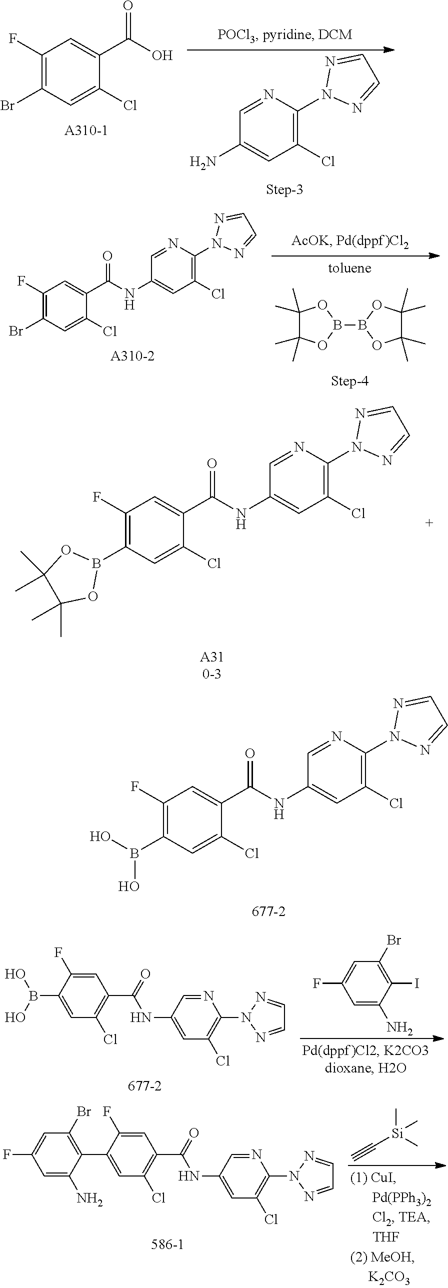

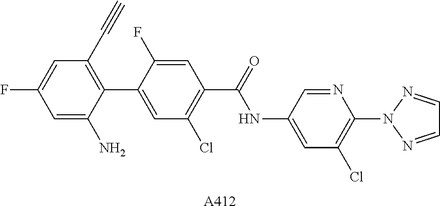 [SMILES: C#Cc1cc(F)cc(N)c1-c1cc(Cl)c(C(=O)Nc2cnc(-n3nccn3)c(Cl)c2)cc1F ; "A412"]

For the synthesis method of 677-2, please refer to the synthesis of A310-3.

### Step 1: Synthesis of Compound A586-1

3-bromo-5-fluoro-2-iodoaniline (0.16 g, 0.5 mmoL), 677-2 (0.12 g, 0.3 mmoL), Pd(dppf)Cl₂ (14.6 mg, 0.02 mmoL) and K₂CO₃ (0.12 g, 0.8 mmoL) were added in sequence to a single-neck bottle containing dioxane (4 mL), H₂O (1 mL) and a stirrer. The reaction liquid container were replaced with nitrogen. The reaction mixture was stirred in a 90° C. oil bath for 8 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product 586-1 (0.065 g, 0.12 mmoL) as white solid. LC-MS (ES⁺): m/z 538/540 [M+H]⁺

### Step 2: Synthesis of Compound A412

ethynyltrimethylsilane (0.06 g, 0.6 mmoL), 586-1 (0.065 g, 0.12 mmoL), CuI (2.0 mg, 0.01 mmoL), Pd(PPh₃)₂Cl₂ (3.5 mg, 0.005 mmoL) and TEA (0.3 g, 3.0 mmoL) were added in sequence to a single-neck bottle containing THF (4 mL) and a stirrer. The reaction liquid container were replaced with nitrogen. The reaction mixture was stirred at room temperature for 5 hours, and then K₂CO₃ (0.42 g, 3 mmoL) and MeOH (10 mL) were added to the reaction mixture and stirring was continued for 2 hours.

After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product A412 (0.05 g, 0.1 mmoL) as white solid. LC-MS (ES⁺): m/z 485[M+H]⁺

### Example 413: Synthesis of Compound A413

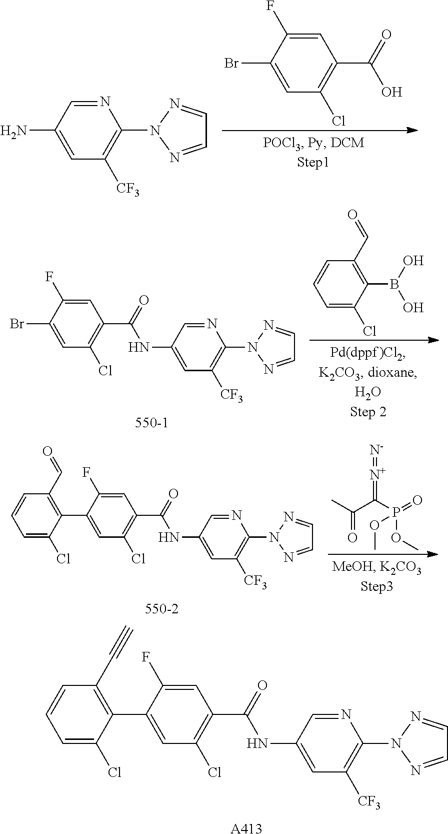

### Step 1: Synthesis of Compound A550-1

Under an ice-water bath, POCl₃ (0.31 g, 2.0 mmoL) was added dropwise to a DCM (10 mL) solution containing 4-bromo-2-chloro-5-fluorobenzoic acid (0.25 g, 1.0 mmoL), 6-(2H-1,2,3-triazol-2-yl)-5-(trifluoromethyl)pyridine-3-amine (0.20 g, 1.0 mmoL) and pyridine (0.84 g, 10 mmoL). The reaction mixture was stirred at room temperature for 2 hours. After the reaction was completed, the reaction solution was diluted with DCM (50 mL), washed with saturated brine (3*50 mL). The organic phase was dried over anhydrous sodium sulfate, concentrated and separated using a Flash silica gel column (PE/EA=2/1) to obtain product 550-1 (0.42 g, 0.9 mmoL) as white solid. LC-MS (ES⁺): m/z 463/465[M+H]⁺

### Step 2: Synthesis of Compound A550-2

(2-chloro-6-formylphenyl)boronic acid (0.092 g, 0.5 mmoL), 550-1 (0.092 g, 0.3 mmoL), Pd(dppf)Cl₂ (14.6 mg, 0.02 mmoL) and K₂CO₃ (0.12 g, 0.8 mmoL) were added in sequence to a single-neck bottle containing dioxane (4 mL), H₂O (1 mL) and a stirrer. The reaction liquid container were replaced with nitrogen. The reaction mixture was stirred in a 90° C. oil bath for 8 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product 550-2 (0.11 g, 0.21 mmoL) as white solid. LC-MS (ES⁺): m/z 524[M+H]⁺

### Step 3: Synthesis of Compound A413

(1-dizo-2-oxopropyl)phosphonate dimethyl ester (0.11 g, 0.5 mmoL), 550-2 (0.11 g, 0.21 mmoL) and K₂CO₃ (0.12 g, 0.8 mmoL) were added in sequence to a single-neck bottle containing MeOH (4 mL) and a stirrer. The reaction mixture was stirred at room temperature for 4 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product A413 (0.08 g, 0.15 mmoL) as white solid. LC-MS (ES⁺): m/z 520[M+H]⁺

### Example 415: Synthesis of Compound A415

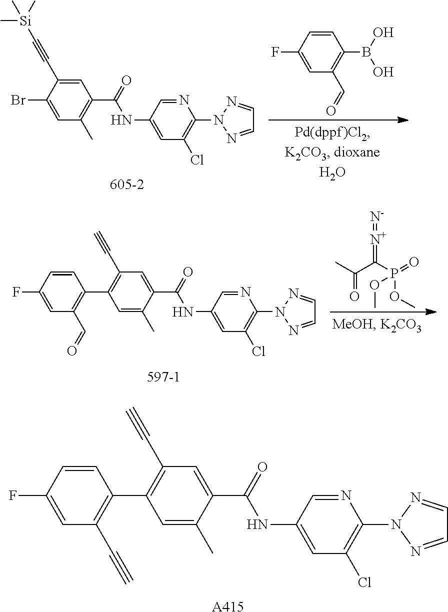 [SMILES: Cc1cc(Br)c(C#C[Si](C)(C)C)cc1C(=O)Nc1cnc(-n2nccn2)c(Cl)c1 ; O=Cc1cc(F)ccc1B(O)O ; C#Cc1cc(C(=O)Nc2cnc(-n3nccn3)c(Cl)c2)c(C)cc1-c1ccc(F)cc1C=O ; COP(=O)(OC)C(=[N+]=[N-])C(C)=O ; C#Cc1cc(F)ccc1-c1cc(C)c(C(=O)Nc2cnc(-n3nccn3)c(Cl)c2)cc1C#C ; "A415"]

### Step 1: Synthesis of Compound A587-1

(4-fluoro-2-formylphenyl)boronic acid (0.09 g, 0.5 mmoL), 605-2 (0.21 g, 0.4 mmoL), Pd(dppf)Cl₂ (14.6 mg, 0.02 mmoL) and K₂CO₃ (0.12 g, 0.8 mmoL) were added in sequence to a single-neck bottle containing dioxane (4 mL), H₂O (1 mL) and a stirrer. The reaction liquid container were replaced with nitrogen. The reaction mixture was stirred in a 90° C. oil bath for 8 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product 597-1 (0.09 g, 0.2 mmoL) as white solid. LC-MS (ES⁺): m/z 460[M+H]⁺

### Step 2: Synthesis of Compound A415

(1-dizo-2-oxopropyl)phosphonate dimethyl ester (0.11 g, 0.5 mmoL), 597-1 (0.09 g, 0.2 mmoL) and K₂CO₃ (0.12 g, 0.8 mmoL) were added in sequence to a single-neck bottle containing MeOH (4 mL) and a stirrer. The reaction mixture was stirred at room temperature for 4 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product A415 (0.06 g, 0.15 mmoL) as white solid. LC-MS (ES⁺): m/z 456[M+H]⁺

### Example 423: Synthesis of Compound A423

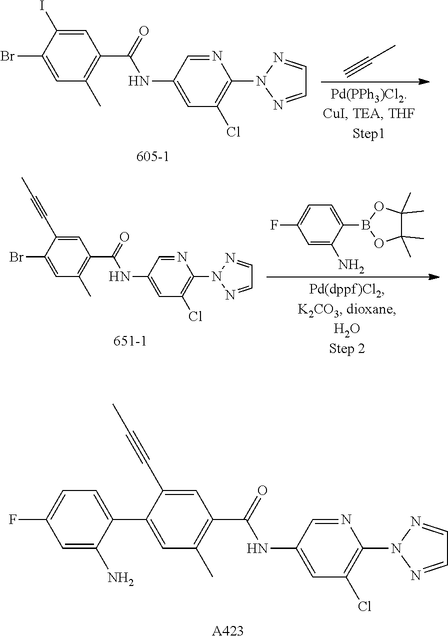

### Step 1: Synthesis of Compound A651-1

propyne (0.1 g, 2.5 mmoL), 605-1 (0.26 g, 0.5 mmoL), CuI (2.0 mg, 0.01 mmoL), Pd(PPh₃)₂Cl₂ (3.5 mg, 0.005 mmoL) and TEA (0.3 g, 3.0 mmoL) were added in sequence to a single-neck bottle containing THF (10 mL) and a stirrer. The reaction liquid container were replaced with nitrogen. The reaction mixture was stirred at room temperature for 5 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product 651-1 (0.13 g, 0.3 mmoL) as white solid. LC-MS (ES⁺): m/z 430/432[M+H]⁺

### Step 2: Synthesis of Compound A423

5-fluoro-2-(4,4,5,5-tetramethyl-1,3,2-dioxaborolan-2-yl) aniline (0.12 g, 0.5 mmoL), 651-1 (0.13 g, 0.3 mmoL), Pd(dppf)Cl₂ (14.6 mg, 0.02 mmoL) and K₂CO₃ (0.12 g, 0.8 mmoL) were added in sequence to a single-neck bottle containing dioxane (4 mL), H₂O (1 mL) and a stirrer. The reaction liquid container were replaced with nitrogen. The reaction mixture was stirred in a 90° C. oil bath for 8 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product A423 (0.09 g, 0.2 mmoL) as white solid. LC-MS (ES⁺): m/z 461[M+H]⁺

### Example 425: Synthesis of Compound A425

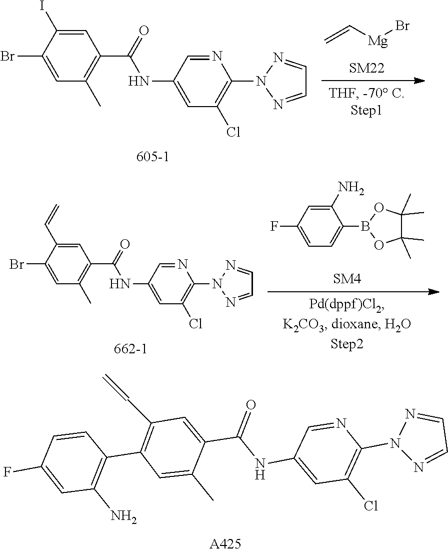

### Step 1: Synthesis of Compound A662-1

Vinyl magnesium bromide (2.0 mL, 2.0 mmoL) was added dropwise to a THF (10 mL) solution containing 605-1 (0.52 g, 1.0 mmoL) at −70° C. The reaction mixture was stirred at −70° C. for 2 hours. After the reaction was completed, the reaction solution was quenched saturated ammonium chloride solution (30 mL) and extracted with DCM (50 mL). The organic phase was washed with saturated brine (3*50 mL) and dried over anhydrous sodium sulfate, concentrated and separated using a Flash silica gel column (PE/EA=2/1) to obtain product 662-1 (0.28 g, 0.7 mmoL) as white solid. LC-MS (ES⁺): m/z 418/420[M+H]⁺

### Step 2: Synthesis of Compound A425

5-fluoro-2-(4,4,5,5-tetramethyl-1,3,2-dioxaborolan-2-yl) aniline (0.14 g, 0.6 mmoL), 662-1 (0.17 g, 0.4 mmoL), Pd(dppf)Cl₂ (14.6 mg, 0.02 mmoL) and K₂CO₃ (0.12 g, 0.8 mmoL) were added in sequence to a single-neck bottle containing dioxane (4 mL), H₂O (1 mL) and a stirrer. The reaction liquid container were replaced with nitrogen. The reaction mixture was stirred in a 90° C. oil bath for 8 hours. After the reaction was completed, the reaction solution was diluted with EA (50 mL), washed with saturated brine (3*50 mL). The organic phase was dried over anhydrous sodium sulfate, concentrated and separated using a Flash silica gel column (PE/EA=2/1) to obtain product A425 (0.12 g, 0.3 mmoL) as white solid. LC-MS (ES⁺): m/z 449 [M+H]⁺.

### Example 510 and 511: Synthesis of Compound A510 and A511

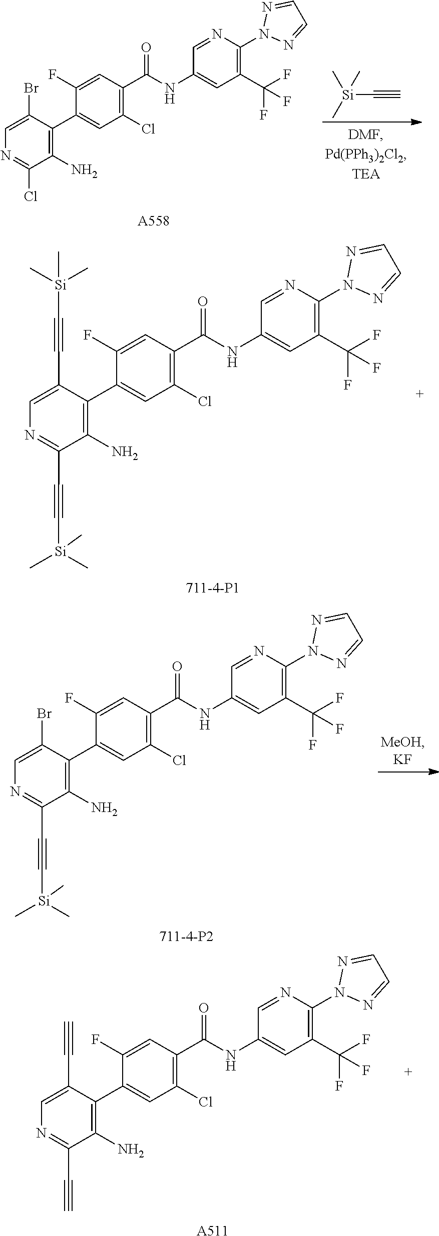

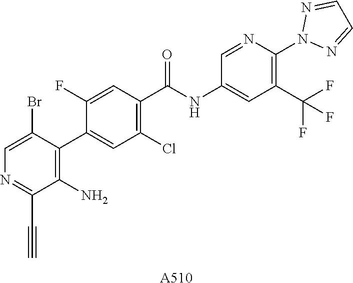 [SMILES: C#Cc1ncc(Br)c(-c2cc(Cl)c(C(=O)Nc3cnc(-n4nccn4)c(C(F)(F)F)c3)cc2F)c1N ; "A510"]

### Step 1: Synthesis of Compound A711-4-P1 and A711-4-P2

Into a 25-mL round-bottom flask, was placed A558 (100 mg, 0.17 mmol), ethynyltrimethylsilane (16.62 mg, 0.17 mmol), Pd(PPh₃)₂Cl₂ (11.86 mg, 0.017 mmol), TEA (34.24 mg, 0.0 mmol), DMF (2 mL). The resulting mixture was stirred at 80° C. for 16 hours under nitrogen. Removing of the solvent and purifying by column chromatography (PE/EA=2/1) to give compound 711-4-P1 (LC-MS (ES⁺): m/z 470[M+H]) and 711-4-P2 (LC-MS (ES⁺): m/z 652/654 [M+H]) mixture (40 mg) as white solid.

### Step 2: Synthesis of Compound A510 and A511

To a solution of 711-4-P1 and 711-4-P2 (40 mg) in MeOH (5.0 mL) was added KF (3.56 mg, 0.06 mmol). The resulting mixture was stirred at 50° C. for 2 hours, solvent was removed in vacuo and the mixture was purified by pre-HPLC to give compound A511 (1.1 mg) (LC-MS (ES⁺): m/z 526 [M+H]⁺) and A510 (1.3 mg) (LC-MS (ES⁺): m/z 579/581 [M+H]0) as white solids.

### Example 520: Synthesis of Compound A520

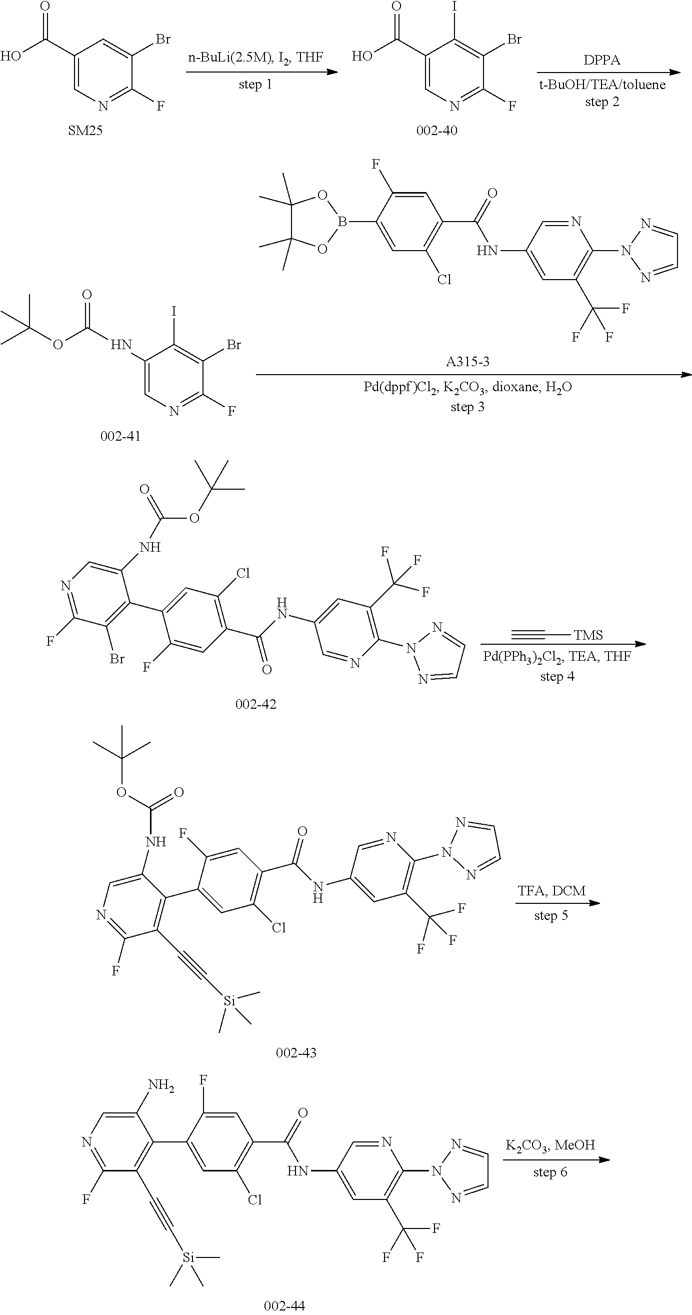

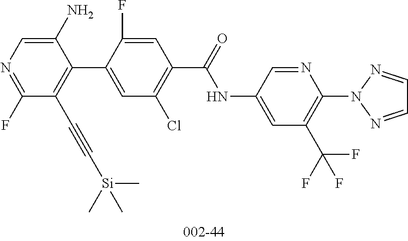 [SMILES: C[Si](C)(C)C#Cc1c(F)ncc(N)c1-c1cc(Cl)c(C(=O)Nc2cnc(-n3nccn3)c(C(F)(F)F)c2)cc1F]

### Step 1: Synthesis of Compound A002-40

Into a 250-mL three-neck flask, 2,2,6,6-tetramethylpiperidine (3.98 g, 28.18 mmol) was dissolved in THF (20 mL) under nitrogen, and n-butyl lithium (2.5 mol/L in THF, 10.9 ml 27.27 mmol) was dropwised at −50° C. Keeping −50° C. for half an hour after dropwise, a solution of 5-bromo-6-fluoronicotinic acid (2.00 g, 9.09 mmol) in THF (20 mL) was dropwised, slowly raising the temperature to −20° C., keeping at −20° C. for 1 h, cooling the reaction to −60° C., 12 (3.46 g, 13.63 mmol) dissolved in THF (20.0 mL) was dropwised. After the reaction completed, it is diluted with 100 mL of water and extracted with EA (3*30 mL). Keep the water phase, adjust the pH to 3 with 3N HCl, then extract with EA (3*50 mL), The organic phase wash with saturated brine (3*30 mL), dry with anhydrous sodium sulfate and concentrate to obtain the product 002-40 (1.5 g, 4.35 mmol) as a red solid. LC-MS (ES⁺): m/z 345/347 [M+H]⁺

### Step 2: Synthesis of Compound A002-41

To a solution of 002-40 (1.5 g, 4.35 mmol) in toluene (15.0 mL) and tert-butanol (15 mL) was added DPPA (1.794 g, 6.52 mmol) and TEA (1.318 g, 13.05 mmol). The resulting mixture was stirred at 110° C. for 2 hours under nitrogen, solvent was removed in vacuo and the mixture was separated on a silica gel column with PE:EA=96:4 to give compound 002-41 (1.5 g, 3.59 mmol) as a brown solid. LC-MS (ES⁺): m/z 416/418 [M+H]⁺

### Step 3: Synthesis of Compound A002-42

To 002-41 (0.6 g, 1.44 mmol) in 1,4-dioxane (6.0 mL) and H₂O (1 mL) was added A315-3 (1.104 g, 2.16 mmoL), Pd(dppf)Cl₂ (105.29 mg, 0.15 mmoL) and K₂CO₃ (0.39 g, 2.88 mmoL). The resulting mixture was stirred at 80° C. for 16 hours under nitrogen. Water was added, and the organic layer was extracted with EtOAc (3*20 mL). The organic layers were combined, washed with brine (3*50 mL), dried over Na₂SO₄, and filtered. The filter was under reduced pressure to give compound 002-42 (0.13 g, 0.19 mmoL) as white solid. LC-MS (ES⁺): m/z 674/676 [M+H]⁺

### Step 4: Synthesis of Compound A002-43

To 002-42 (70 mg, 0.1 mmol) in THF (2.0 mL) was added ethynyltrimethylsilane (12.25 mg, 0.12 mmol), Pd(PPh₃)₂Cl₂ (7.3 mg, 0.01 mmol) and TEA (20.99 mg, 0.21 mmol). The resulting mixture was stirred at 80° C. for 3 hours under nitrogen. Water was added, and the organic layer was extracted with EtOAc (3*20 mL). The organic layers were combined, washed with brine (3*50 mL), dried over Na₂SO₄, and filtered. The filter was under reduced pressure and the mixture was separated on a silica gel column with PE:EA=2/1 to give compound 002-43 (85 mg, 0.12 mmol) as yellow oil. LC-MS (ES⁺): m/z 692 [M+H]⁺

### Step 5: Synthesis of Compound A002-44

To 002-43 (85 mg, 0.12 mmol) in DCM (2.0 mL) was added TFA (0.14 mL). The resulting mixture was stirred at room temperature for 2 hours, after the reaction completed, it is diluted with EA (20 mL). The mixture was adjusted the pH to 9 with saturated NaHCO₃ solution, then extract with EA (2*30 mL), The organic phase wash with saturated brine (3*30 mL), dry with anhydrous sodium sulfate and filtered. The filter was under reduced pressure to give compound 002-44 (70 mg, 0.12 mmol) as yellow solid. LC-MS (ES⁺): m/z 592 [M+H]⁺

### Step 6: Synthesis of Compound A520

To 002-44 (60 mg, 0.1 mmol) in MeOH (2.0 mL) was added K₂CO₃ (19.60 mg, 0.14 mmol). The resulting mixture was stirred at 50° C. for 10 minutes, solvent was removed in vacuo and the mixture was separated on pre-HPLC to give compound A520 (12 mg, 0.023 mmol) as white solid. LC-MS (ES⁺): m/z 520 [M+H]⁺

### Example 553: Synthesis of Compound A553

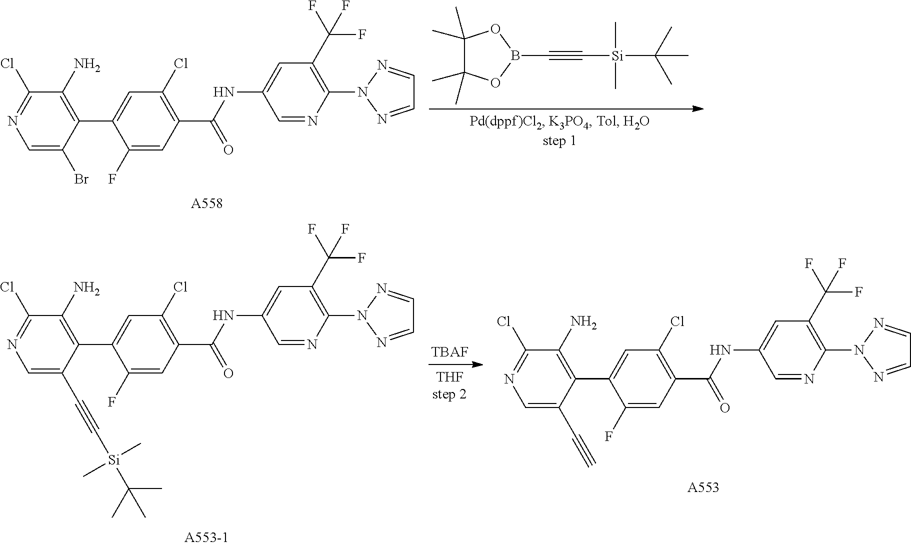

### Step 1: Synthesis of Compound A553-1

To A558 (60.0 mg, 0.102 mmol) in toluene (10.0 mL) and H₂O (1.0 mL) was added tert-butyldimethyl ((4,4,5,5-tetramethyl-1,3,2-dioxobenzaldehyde-2-yl)ethynyl)silane (50.0 mg, 0.36 mmol), [PdCl₂(dppf)]CH₂Cl₂ (30 mg, 0.037 mmol) and K₂CO₃ (0.39 g, 2.88 mmol). The resulting mixture was stirred at 95° C. for 16 hours under nitrogen. Water was added, and the organic layer was extracted with EtOAc (3*20 mL). The organic layers were combined, washed with brine (3*50 mL), dried over Na₂SO₄, and filtered. The filter was under reduced pressure to give compound A553-1 (35 mg, 0.054 mmol) as white solid. LC-MS (ES⁺): m/z 650.25 [M+H]⁺

### Step 2: Synthesis of Compound A553

To A553-1 (200 mg, 0.308 mmol) in THF (10.0 mL) was added TBAF (0.1M in THF, 1.0 ml). The resulting mixture was stirred at room temperature for 30 minutes, solvent was removed in vacuo and the mixture was separated on pre-HPLC to give compound A553 (92 mg, 0.0.172 mmol) as white solid. LC-MS (ES⁺): m/z 536.10 [M+H]⁺.

¹H NMR (500 MHz, DMSO-d₆) δ 11.57 (s, 1H), 9.16 (d, J=2.4 Hz, 1H), 8.91 (d, J=2.4 Hz, 1H), 8.22 (s, 2H), 7.90-7.84 (m, 2H), 7.70 (d, J=6.1 Hz, 1H), 5.59 (s, 2H), 4.33 (s, 1H).

### Example 555: Synthesis of Compound A555

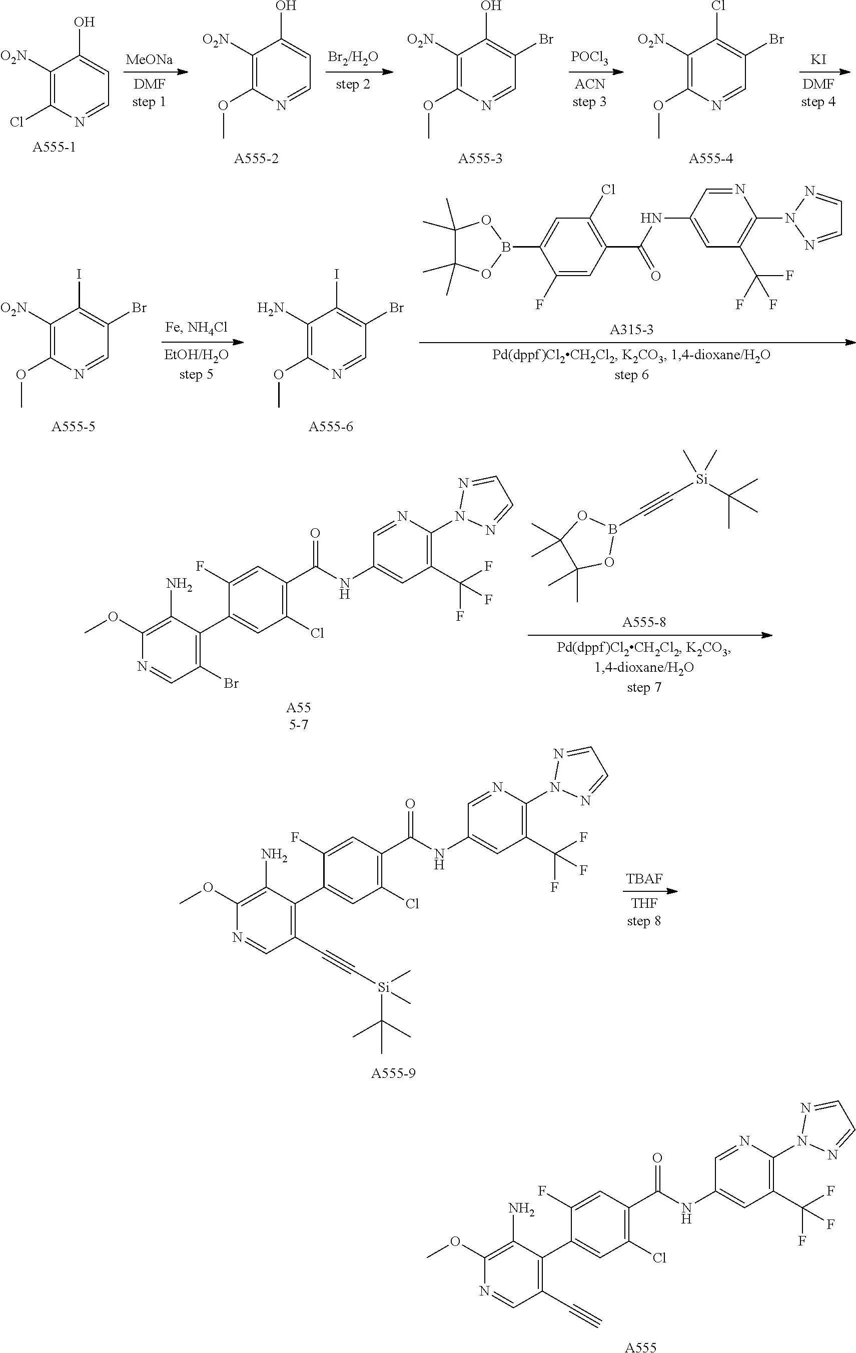

### Step 1: Synthesis of Compound A555-2

To A555-1 (1.00 g, 5.73 mmol) in DMF (10.0 mL) was added sodium methoxide (5.16 g, 30% wt, 28.65 mmol). The resulting mixture was stirred at 100° C. for 2.5 hours. Water was added, and the organic layer was extracted with EtOAc (3*20 mL). The organic layers were combined, washed with brine (3*50 mL), dried over Na₂SO₄, and filtered. The filter was under reduced pressure to give compound A555-2 (0.90 g, 92.34% yield) as a yellow powder. LC-MS (ES⁺): m/z 171.0 [M+H]⁺.

### Step 2: Synthesis of Compound A555-3

To A555-2 (0.500 g, 2.94 mmol) in glacial acetic acid (10.0 mL) was added a solution of bromine (0.17 mL, 3.23 mmol) in glacial acetic acid (5 mL) slowly at room temperature. The resulting mixture was stirred at room temperature for 16 hours. Water was added, the mixture is filtered, collect the filter cake and dry to give compound A555-3 (465 mg, 63.53% yield) as a yellow solid. LC-MS (ES⁺): m/z 249/251 [M+H]⁺.

### Step 3: Synthesis of Compound A555-4

To A555-3 (115 mg, 0.46 mmol) in acetonitrile (2.0 mL) was added phosphorus oxychloride (0.22 mL, 2.31 mmol) at room temperature. The resulting mixture was stirred at 85° C. for 2 hours. Water was added, the mixture is filtered, collect the filter cake and dry to give compound A555-4 (55 mg, 44.53% yield) as a yellow solid. LC-MS (ES⁺): m/z 249/251 [M+H]⁺.

### Step 4: Synthesis of Compound A555-5

To A555-4 (200 mg, 0.75 mmol) in DMF (5.0 mL) was added potassium iodide (1.24 g, 7.48 mmol). The resulting mixture was stirred at 100° C. for 5 hours. Water was added, and the organic layer was extracted with EtOAc (3*20 mL). The organic layers were combined, washed with brine (3*50 mL), dried over Na₂SO₄, and filtered. The filter was under reduced pressure the mixture was separated on a silica gel column with PE:EA=1/1 to give compound A555-5 (78 mg, 29.06% yield) as a yellow solid. LC-MS (ES⁺): m/z 359/361 [M+H]⁺.

### Step 5: Synthesis of Compound A555-6

To A555-5 (240 mg, 0.67 mmol) in EtOH/H₂O (5 mL/1 mL) was added Fe(187 mg, 3.34 mmol) and NH₄Cl (179 mg, 3.34 mmol). The resulting mixture was stirred at 60° C. for 4 hours. The mixture was filtered. The filter was under reduced pressure the mixture was separated on a silica gel column with PE:EA=3/2 to give compound A555-6 (185 mg, 71.49% yield) as a yellow solid. LC-MS (ES⁺): m/z 329/331 [M+H]⁺.

### Step 6: Synthesis of Compound A555-7

To A558 (60.0 mg, 0.102 mmol) in 1,4-dioxane/water (5 mL/1 mL) was added A315-3 (418 mg, 0.97 mmoL), Pd(dppf)Cl₂ (60 mg, 0.07 mmoL) and K₂CO₃ (134 mg, 0.97 mmoL). The resulting mixture was stirred at 80° C. for 2 hours under nitrogen. Water was added, and the organic layer was extracted with EtOAc (3*20 mL). The organic layers were combined, washed with brine (3*50 mL), dried over Na₂SO₄, and filtered. The filter was under reduced pressure and the mixture separated on a silica gel column with PE:EA=0/1 to give compound A555-7 (120 mg, 42.05% yield) as a yellow solid. LC-MS (ES⁺): m/z 586/588 [M+H]⁺

### Step 7: Synthesis of Compound A555-9

To A555-7 (100 mg, 0.17 mmol) in 1,4-dioxane/water (2 mL/0.2 mL) was added A555-8 (68 mg, 0.26 mmoL), Pd(dppf)Cl₂·CH₂Cl₂ (28 mg, 0.03 mmoL) and K₂CO₃ (71 mg, 0.51 mmoL). The resulting mixture was stirred at 80° C. for 6 hours under nitrogen. Water was added, and the organic layer was extracted with EtOAc (3*20 mL). The organic layers were combined, washed with brine (3*50 mL), dried over Na₂SO₄, and filtered. The filter was under reduced pressure and the mixture separated on a silica gel column with DCM/MeOH=20/1 to give compound A555-9 (25 mg, 22.71% yield) as a yellow solid. LC-MS (ES⁺): m/z 646 [M+H]⁺

### Step 8: Synthesis of Compound A555

To A555-9 (25 mg, 0.04 mmol) in THF (1.0 mL) was added TBAF (0.1M in THF, 1.0 ml). The resulting mixture was stirred at room temperature for 60 minutes, solvent was removed in vacuo and the mixture was separated on pre-HPLC to give compound A555 (6.6 mg, 32.07%) as a yellow solid. LC-MS (ES⁺): m/z 532[M+H]⁺.

¹H NMR (500 MHz, DMSO-d₆) δ 11.50 (s, 1H), 9.17 (d, J=2.4 Hz, 1H), 8.89 (d, J=2.4 Hz, 1H), 8.22 (s, 2H), 7.89 (d, J=9.0 Hz, 2H), 7.74 (d, J=6.0 Hz, 1H), 5.82 (s, 2H), 4.08 (s, 1H), 3.83 (s, 3H).

### Example 558: Synthesis of Compound A558

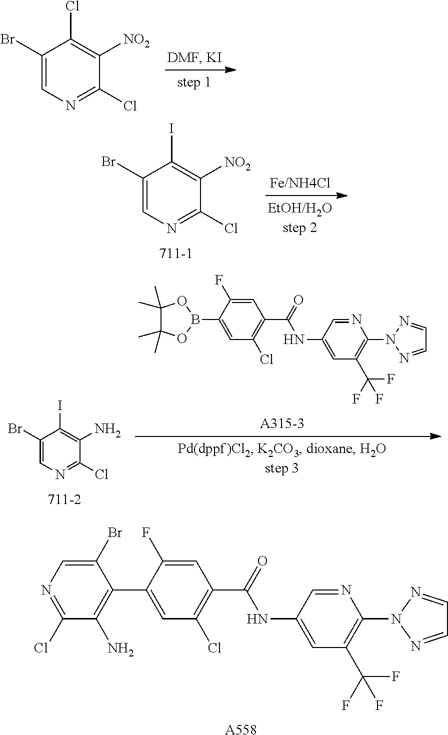 [SMILES: O=[N+]([O-])c1c(Cl)ncc(Br)c1Cl ; O=[N+]([O-])c1c(Cl)ncc(Br)c1I ; CC1(C)OB(c2cc(Cl)c(C(=O)Nc3cnc(-n4nccn4)c(C(F)(F)F)c3)cc2F)OC1(C)C ; Nc1c(Cl)ncc(Br)c1I ; Nc1c(Cl)ncc(Br)c1-c1cc(Cl)c(C(=O)Nc2cnc(-n3nccn3)c(C(F)(F)F)c2)cc1F ; "A558"]

### Step 1: Synthesis of Compound A711-1

To 5-bromo-2,4-dichloro-3-nitropyridine (2 g, 7.35 mmol) in DMF (20.0 mL) was added KI (3.67 g, 22.05 mmol). The resulting mixture was stirred at 100° C. for 16 hours. Water was added, the mixture is filtered, the filter cake wash with ice water (3*10 mL), collect the filter cake and dry to give compound 771-1 (2.7 g, 7.43 mmol) as a gray solid. LC-MS (ES⁺): m/z 362/364 [M+H]⁺.

### Step 2: Synthesis of Compound A711-2

To 711-1 (2.7 g, 7.43 mmoL) in EtOH (20.0 mL) and H2O (20.0 mL) was added Fe2.08 g, 37.2 mmoL) and NH₄Cl (2.0 g, 37.2 mmol). The resulting mixture was stirred at 40° C. for 3 hours. The mixture was filtered. The filter was extracted with EtOAc (3*50 mL). The organic layers were combined, washed with saturated sodium bicarbonate solution (100 ml) and brine (100 mL), dried over Na₂SO₄, and filtered. The filter was under reduced pressure and the mixture separated on a silica gel column with PE/EA=90/10 to give compound 711-2 (1.2 g, 3.6 mmoL) as a white solid. LC-MS (ES⁺): m/z 332/334 [M+H]⁺.

### Step 3: Synthesis of Compound A558

To A315-3 (2.76 g, 5.4 mmoL) in 1,4-dioxane/water (30 mL/3.0 mL) was added 711-2 (1.2 g, 3.6 mmoL), Pd(dppf)Cl₂ (26.35 mg, 0.036 mmoL) and K₂CO₃ (0.99 g, 7.2 mmoL). The resulting mixture was stirred at 80° C. for 16 hours under nitrogen. Water was added, and the organic layer was extracted with EtOAc (3*20 mL). The organic layers were combined, washed with brine (3*50 mL), dried over Na₂SO₄, and filtered. The filter was under reduced pressure and the mixture separated on a silica gel column with DCM/EA=3/1 to give compound A558 (0.48 g, 0.8 mmoL) as white solid. LC-MS (ES⁺): m/z 589/591 [M+H]⁺.

### Example 559 and 560: Synthesis of Compound A559 and A560

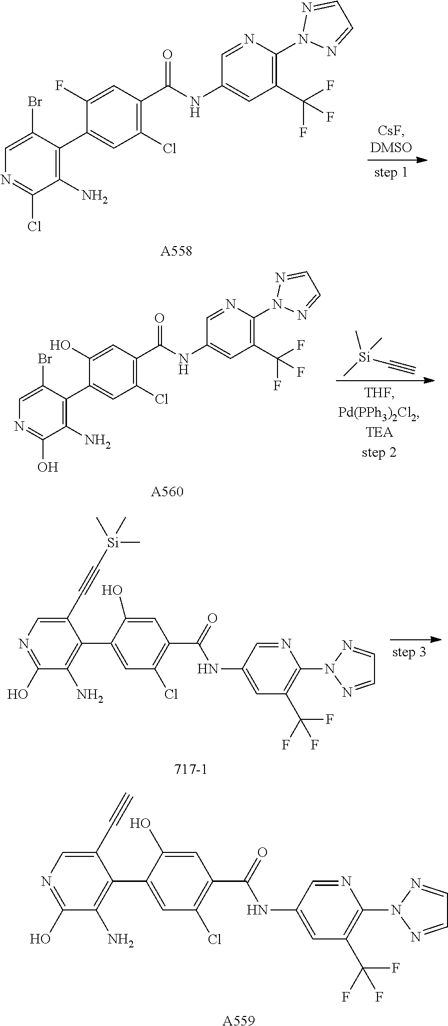

### Step 1: Synthesis of Compound A560

To A558 (100 mg, 0.17 mmol) in DMSO (2.0 mL) was added CSF (51.40 mg, 0.34 mmol). The resulting mixture was stirred at 130° C. for 16 hours. Water was added, and the organic layer was extracted with EtOAc (3*20 mL). The organic layers were combined, washed with brine (3*50 mL), dried over Na₂SO₄, and filtered. The filter was under reduced pressure to give compound A560 (15 mg, 0.026 mmol) as white solid. LC-MS (ES⁺): m/z 569/571 [M+H]⁺.

### Step 2: Synthesis of Compound A717-1

To A560 (15 mg, 0.17 mmol) in THF (2.0 mL) was added ethynyltrimethylsilane (2.6 mg, 0.026 mmol), Pd(PPh₃)₂Cl₂ (1.8 mg, 0.0026 mmol) and TEA (5.3 mg, 0.05 mmol). The resulting mixture was stirred at 80° C. for 16 hours under nitrogen.

Water was added, and the organic layer was extracted with EtOAc (3*10 mL). The organic layers were combined, washed with brine (3*10 mL), dried over Na₂SO₄, and filtered. The filter was under reduced pressure to give compound 717-1 (20 mg, 0.03 mmol) as white solid. LC-MS (ES⁺): m/z 588 [M+H]⁺.

### Step 3: Synthesis of Compound A559

To 717-1 (20 mg, 0.03) in MeOH (1.0 mL) was added K₂CO₃ (9.40 mg, 0.07 mmol). The resulting mixture was stirred at 50° C. for 2 hours, solvent was removed in vacuo and the mixture was separated on pre-HPLC to give compound A559 (2.0 mg) as a white solid. LC-MS (ES⁺): m/z 516 [M+H]⁺.

According to the synthesis method of the above example, select appropriate raw materials and/or marketed example intermediates to synthesize the example compounds in the table below. The raw materials and reagents used are all commercially available.

| | | | MS |
|---|---|---|---|
| Example | Structure | Chemical Name | [M + H]⁺ |
| Example 410 (A410) | 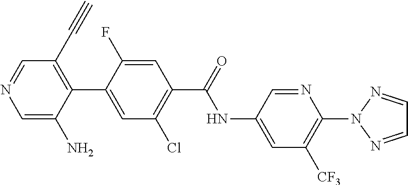 [SMILES: C#Cc1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5- ethynylpyridin-4-yl)-2- chloro-5-fluorobenzamide | 502 |
| Example 411 (A411) | 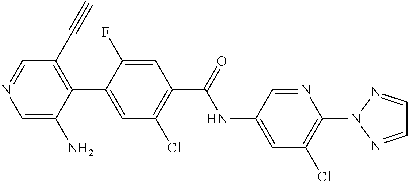 [SMILES: C#Cc1cncc(N)c1-c1cc(Cl)c(C(=O)Nc2cnc(-n3nccn3)c(Cl)c2)cc1F] | 4-(3-amino-5- ethynylpyridin-4-yl)-2- chloro-N-(5-chloro-5-(2H- 1,2,3-triazol-2-yl)pyridin-3- yl)-5-fluorobenzamide | 468 |
| Example 414 (A414) | 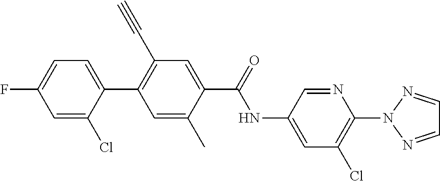 [SMILES: C#Cc1cc(C(=O)Nc2cnc(-n3nccn3)c(Cl)c2)c(C)cc1-c1ccc(F)cc1Cl] | 2′-chloro-N-(5-chloro-6- (2H-1,2,3-triazol-2- yl)pyridin-3-yl)-2-ethynyl- 4′-fluoro-5-methyl-[1,1′- biphenyl]-4-carboxamide | 466 |
| Example 416 (A416) | 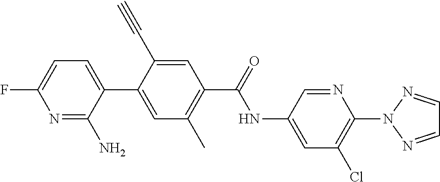 [SMILES: C#Cc1cc(C(=O)Nc2cnc(-n3nccn3)c(Cl)c2)c(C)cc1-c1ccc(F)nc1N] | 4-(2-amino-6-fluoropyridin- 3-yl)-N-(5-chloro-6-(2H- 1,2,3-triazol-2-yl)pyridin-3- yl)-5-ethynyl-2- methylbenzamide | 448 |
| Example 417 (A417) | 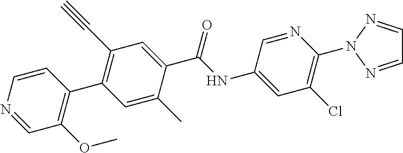 [SMILES: C#Cc1cc(C(=O)Nc2cnc(-n3nccn3)c(Cl)c2)c(C)cc1-c1ccncc1OC] | N-(5-chloro-6-(2H-1,2,3- triazol-2-yl)pyridin-3-yl)-5- ethynyl-4-(3- methoxypyridin-4-yl)-2- methylbenzamide | 445 |
| Example 418 (A418) | 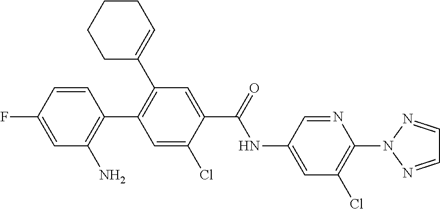 [SMILES: Nc1cc(F)ccc1-c1cc(Cl)c(C(=O)Nc2cnc(-n3nccn3)c(Cl)c2)cc1C1=CCCCC1] | 2-amino-5′-chloro-N-(5- chloro-6-(2H-1,2,3-triazol-2- yl)pyridin-3-yl)-4-fluoro- 2″,3″,4″,5″-tetrahydro- [1,1′:2′,1″-terphenyl]-4′- carboxamide | 523 |
| Example 419 (A419) | 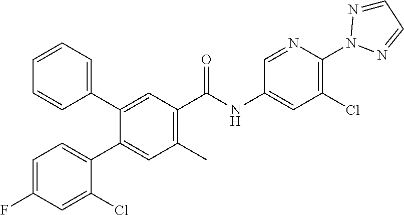 [SMILES: Cc1cc(-c2ccc(F)cc2Cl)c(-c2ccccc2)cc1C(=O)Nc1cnc(-n2nccn2)c(Cl)c1] | 2-chloro-N-(5-chloro-6-(2H- 1,2,3-triazol-2-yl)pyridin-3- yl)-4-fluoro-5′-methyl- [1,1′:2′,1″-terphenyl]-4′- carboxamide | 518 |
| Example 420 (A420) | 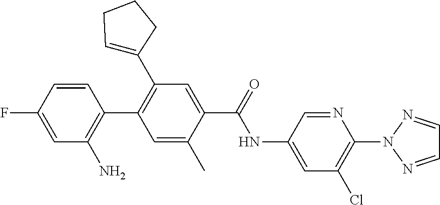 [SMILES: Cc1cc(-c2ccc(F)cc2N)c(C2=CCCC2)cc1C(=O)Nc1cnc(-n2nccn2)c(Cl)c1] | 2′-amino-N-(5-chloro-6-(2H- 1,2,3-triazol-2-yl)pyridin-3- yl)-2-(cyclopent-1-en-1-yl)- 4′-fluoro-5-methyl-[1,1′- biphenyl]-4-carboxamide | 489 |
| Example 421 (A421) | 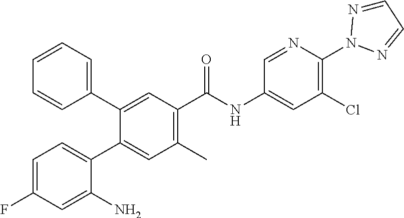 [SMILES: Cc1cc(-c2ccc(F)cc2N)c(-c2ccccc2)cc1C(=O)Nc1cnc(-n2nccn2)c(Cl)c1] | 2-amino-N-(5-chloro-6-(2H- 1,2,3-triazol-2-yl)pyridin-3- yl)-4-fluoro-5′-methyl- [1,1′:2′,1″-terphenyl]-4′- carboxamide | 499 |
| Example 422 (A422) | 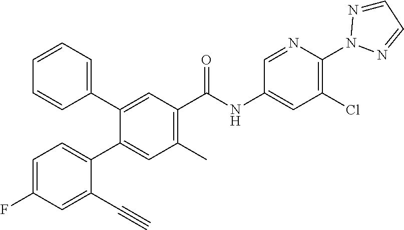 [SMILES: C#Cc1cc(F)ccc1-c1cc(C)c(C(=O)Nc2cnc(-n3nccn3)c(Cl)c2)cc1-c1ccccc1] | N-(5-chloro-6-(2H-1,2,3- triazol-2-yl)pyridin-3-yl)-2- ethynyl-4-fluoro-5′-methyl- [1,1′:2′,1″-terphenyl]-4′- carboxamide | 508 |
| Example 424 (A424) | 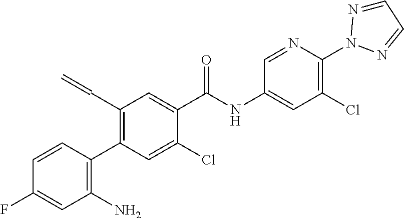 [SMILES: C=Cc1cc(C(=O)Nc2cnc(-n3nccn3)c(Cl)c2)c(Cl)cc1-c1ccc(F)cc1N] | 2′-amino-5-chloro-N-(5- chloro-6-(2H-1,2,3-triazol-2- yl)pyridin-3-yl)-4′-fluoro-2- vinyl-[1,1′-biphenyl]-4- carboxamide | 469 |
| Example 426 (A426) | 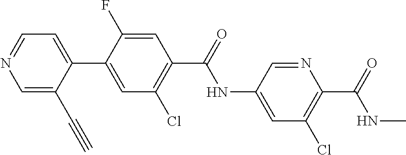 [SMILES: C#Cc1cnccc1-c1cc(Cl)c(C(=O)Nc2cnc(C(=O)NC)c(Cl)c2)cc1F] | 3-chloro-5-(2-chloro-4-(3- ethynylpyridin-4-yl)-5- fluorobenzamido)-N- methylpicolinamide | 443 |
| Example 427 (A427) | 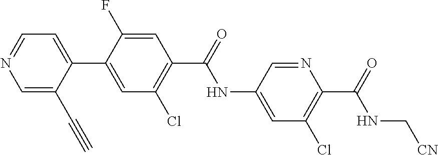 [SMILES: C#Cc1cnccc1-c1cc(Cl)c(C(=O)Nc2cnc(C(=O)NCC#N)c(Cl)c2)cc1F] | 3-chloro-5-(2-chloro-4-(3- ethynylpyridin-4-yl)-5- fluorobenzamido)-N- (cyanomethyl)picolinamide | 468 |
| Example 428 (A428) | 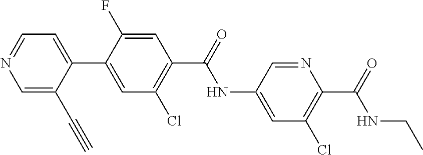 [SMILES: C#Cc1cnccc1-c1cc(Cl)c(C(=O)Nc2cnc(C(=O)NCC)c(Cl)c2)cc1F] | 3-chloro-5-(2-chloro-4-(3- ethynylpyridin-4-yl)-5- fluorobenzamido)-N- ethylpicolinamide | 457 |
| Example 429 (A429) | 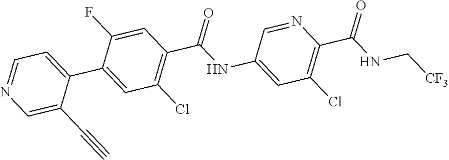 [SMILES: C#Cc1cnccc1-c1cc(Cl)c(C(=O)Nc2cnc(C(=O)NCC(F)(F)F)c(Cl)c2)cc1F] | 3-chloro-5-(2-chloro-4-(3- ethynylpyridin-4-yl)-5- fluorobenzamido)-N-(2,2,2- trifluoroethyl)picolinamide | 511 |
| Example 430 (A430) | 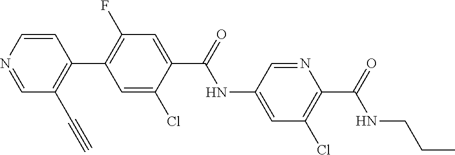 [SMILES: C#Cc1cnccc1-c1cc(Cl)c(C(=O)Nc2cnc(C(=O)NCCC)c(Cl)c2)cc1F] | 3-chloro-5-(2-chloro-4-(3- ethynylpyridin-4-yl)-5- fluorobenzamido)-N- propylpicolinamide | 471 |
| Example 431 (A431) | 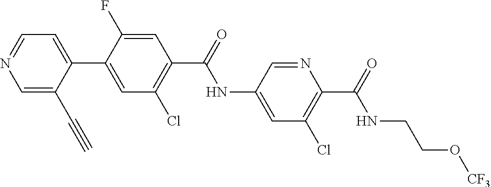 [SMILES: C#Cc1cnccc1-c1cc(Cl)c(C(=O)Nc2cnc(C(=O)NCCOC(F)(F)F)c(Cl)c2)cc1F] | 3-chloro-5-(2-chloro-4-(3- ethynylpyridin-4-yl)-5- fluorobenzamido)-N-(2- (trifluoromethoxy)ethyl) picolinamide | 541 |
| Example 432 (A432) | 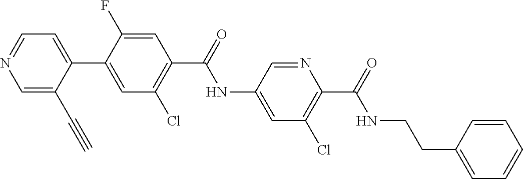 [SMILES: C#Cc1cnccc1-c1cc(Cl)c(C(=O)Nc2cnc(C(=O)NCCc3ccccc3)c(Cl)c2)cc1F] | 3-chloro-5-(2-chloro-4-(3- ethynylpyridin-4-yl)-5- fluorobenzamido)-N- phenethylpicolinamide | 533 |
| Example 433 (A433) | 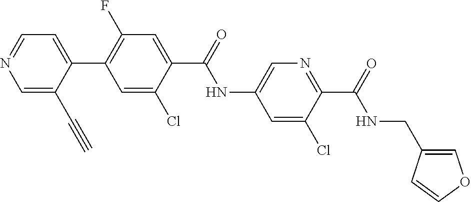 [SMILES: C#Cc1cnccc1-c1cc(Cl)c(C(=O)Nc2cnc(C(=O)NCc3ccoc3)c(Cl)c2)cc1F] | 3 3-chloro-5-(2-chloro-4-(3- ethynylpyridin-4-yl)-5- fluorobenzamido)-N-(furan- 3-ylmethyl)picolinamide | 509 |
| Example 434 (A434) | 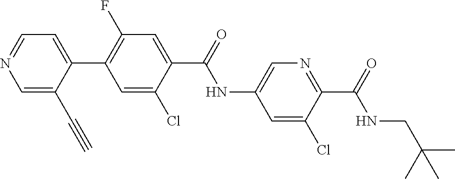 [SMILES: C#Cc1cnccc1-c1cc(Cl)c(C(=O)Nc2cnc(C(=O)NCC(C)(C)C)c(Cl)c2)cc1F] | 3-chloro-5-(2-chloro-4-(3- ethynylpyridin-4-yl)-5- fluorobenzamido)-N- neopentylpicolinamide | 499 |
| Example 435 (A435) | 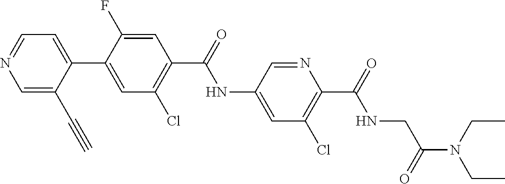 [SMILES: C#Cc1cnccc1-c1cc(Cl)c(C(=O)Nc2cnc(C(=O)NCC(=O)N(CC)CC)c(Cl)c2)cc1F] | 3-chloro-5-(2-chloro-4-(3- ethynylpyridin-4-yl)-5- fluorobenzamido)-N-(2- (diethylamino)-2- oxoethyl)picolinamide | 542 |
| Example 436 (A436) | 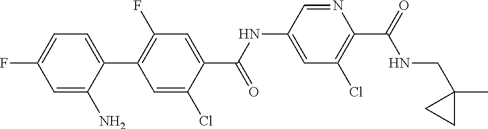 [SMILES: CC1(CNC(=O)c2ncc(NC(=O)c3cc(F)c(-c4ccc(F)cc4N)cc3Cl)cc2Cl)CC1] | 5-(2′-amino-5-chloro-2,4′- difluoro-[1,1′-biphenyl]-4- carboxamido)-3-chloro-N- ((1- methylcyclopropyl)methyl) picolinamide | 505 |
| Example 437 (A437) | 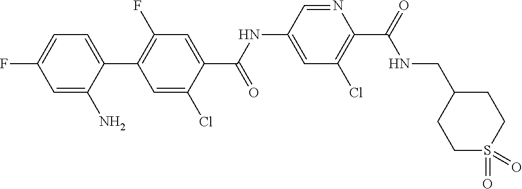 [SMILES: Nc1cc(F)ccc1-c1cc(Cl)c(C(=O)Nc2cnc(C(=O)NCC3CCS(=O)(=O)CC3)c(Cl)c2)cc1F] | 5-(2′-amino-5-chloro-2,4′- difluoro-[1,1′-biphenyl]-4- carboxamido)-3-chloro-N- ((1,1-dioxidotetrahydro-2H- thiopyran-4- yl)methyl)picolinamide | 583 |
| Example 438 (A438) | 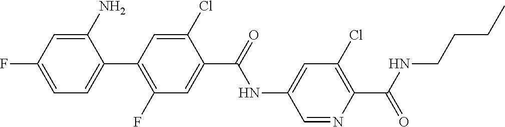 [SMILES: CCCCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-N-butyl-3- chloropicolinamide | 493 |
| Example 439 (A439) | 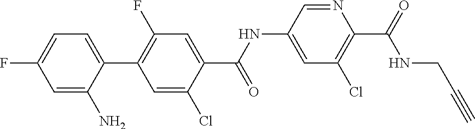 [SMILES: C#CCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- (prop-2-yn-1-yl) picolinamide | 475 |
| Example 440 (A440) | 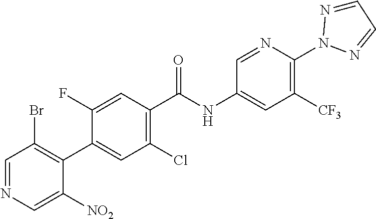 [SMILES: O=C(Nc1cnc(-n2nccn2)c(C(F)(F)F)c1)c1cc(F)c(-c2c(Br)cncc2[N+](=O)[O-])cc1Cl] | N-(6-(2H-1,2,3-triazol-2-yl)- 5-(trifluoromethyl)pyridin-3- yl)-4-(3-bromo-5- nitropyridin-4-yl)-2-chloro- 5-fluorobenzamide | 586 |
| Example 441 (A441) | 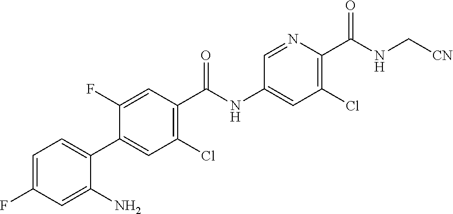 [SMILES: N#CCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- (cyanomethyl)picolinamide | 476 |
| Example 442 (A442) | 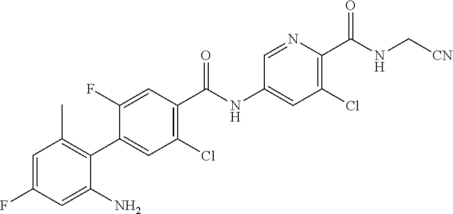 [SMILES: Cc1cc(F)cc(N)c1-c1cc(Cl)c(C(=O)Nc2cnc(C(=O)NCC#N)c(Cl)c2)cc1F] | 5-(2′-amino-5-chloro-2,4′- difluoro-6′-methyl-[1,1′- biphenyl]-4-carboxamido)-3- chloro-N- (cyanomethyl)picolinamide | 490 |
| Example 443 (A443) | 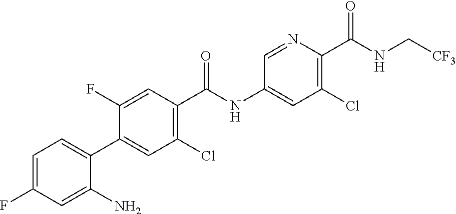 [SMILES: Nc1cc(F)ccc1-c1cc(Cl)c(C(=O)Nc2cnc(C(=O)NCC(F)(F)F)c(Cl)c2)cc1F] | 5-(2′-amino-5-chloro-2,4′- difluoro-[1,1′-biphenyl]-4- carboxamido)-3-chloro-N- (2,2,2- trifluoroethyl)picolinamide | 519 |
| Example 444 (A444) | 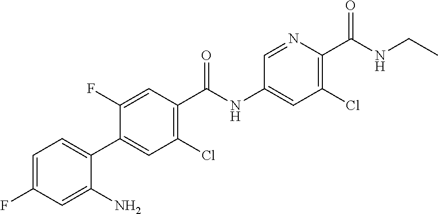 [SMILES: CCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- ethylpicolinamide | 465 |
| Example 445 (A445) | 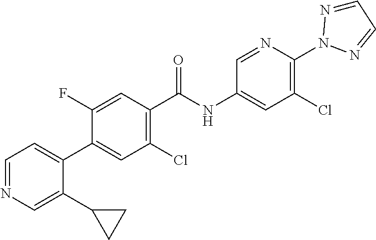 [SMILES: O=C(Nc1cnc(-n2nccn2)c(Cl)c1)c1cc(F)c(-c2ccncc2C2CC2)cc1Cl] | 2-chloro-N-(5-chloro-6-(2H- 1,2,3-triazol-2-yl)pyridin-3- yl)-4-(3-cyclopropylpyridin- 4-yl)-5-fluorobenzamide | 469 |
| Example 446 (A446) | 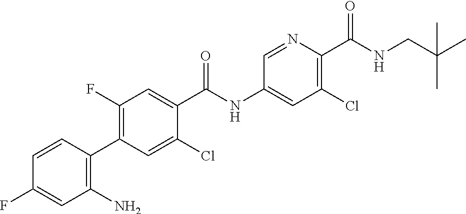 [SMILES: CC(C)(C)CNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- neopentylpicolinamide | 507 |
| Example 447 (A447) | 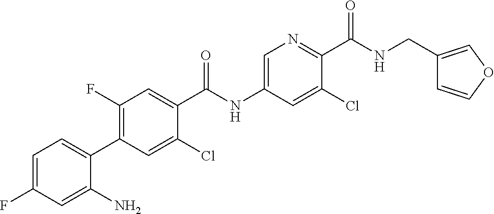 [SMILES: Nc1cc(F)ccc1-c1cc(Cl)c(C(=O)Nc2cnc(C(=O)NCc3ccoc3)c(Cl)c2)cc1F] | 5-(2′-amino-5-chloro-2,4′- difluoro-[1,1′-biphenyl]-4- carboxamido)-3-chloro-N- (furan-3- ylmethyl)picolinamide | 517 |
| Example 448 (A448) | 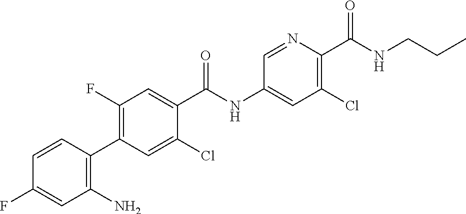 [SMILES: CCCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- propylpicolinamide | 479 |
| Example 449 (A449) | 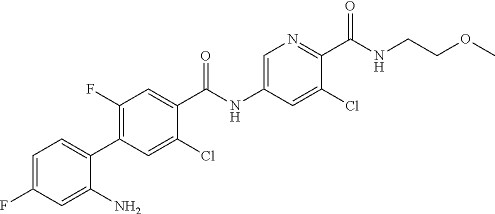 [SMILES: COCCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- (2- methoxyethyl)picolinamide | 495 |
| Example 450 (A450) | 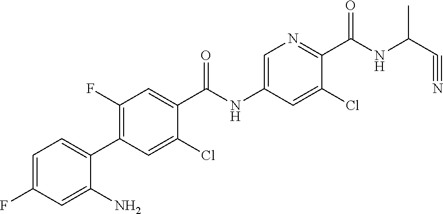 [SMILES: CC(C#N)NC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- (1-cyanoethyl)picolinamide | 490 |
| Example 451 (A451) | 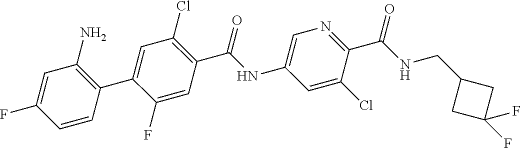 [SMILES: Nc1cc(F)ccc1-c1cc(Cl)c(C(=O)Nc2cnc(C(=O)NCC3CC(F)(F)C3)c(Cl)c2)cc1F] | 5-(2′-amino-5-chloro-2,4′- difluoro-[1,1′-biphenyl]-4- carboxamido)-3-chloro-N- ((3,3- difluorocyclobutyl)methyl) picolinamide | 541 |
| Example 452 (A452) | 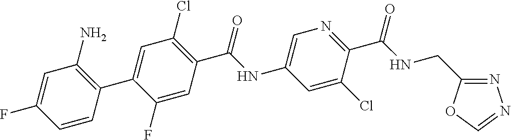 [SMILES: Nc1cc(F)ccc1-c1cc(Cl)c(C(=O)Nc2cnc(C(=O)NCc3nnco3)c(Cl)c2)cc1F] | N-((1,3,4-oxadiazol-2- yl)methyl)-5-(2′-amino-5- chloro-2,4′-difluoro-[1,1′- biphenyl]-4-carboxamido)-3- chloropicolinamide | 519 |
| Example 453 (A453) | 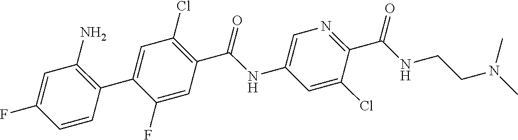 [SMILES: CN(C)CCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- (2- (dimethylamino)ethyl) picolinamide | 508 |
| Example 454 (A454) | 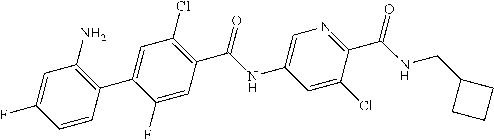 [SMILES: Nc1cc(F)ccc1-c1cc(Cl)c(C(=O)Nc2cnc(C(=O)NCC3CCC3)c(Cl)c2)cc1F] | 5-(2′-amino-5-chloro-2,4′- difluoro-[1,1′-biphenyl]-4- carboxamido)-3-chloro-N- (cyclobutylmethyl) picolinamide | 505 |
| Example 455 (A455) | 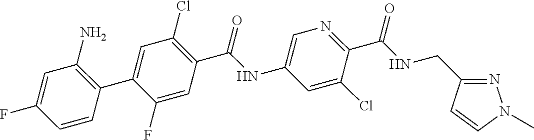 [SMILES: Cn1ccc(CNC(=O)c2ncc(NC(=O)c3cc(F)c(-c4ccc(F)cc4N)cc3Cl)cc2Cl)n1] | 5-(2′-amino-5-chloro-2,4′- difluoro-[1,1′-biphenyl]-4- carboxamido)-3-chloro-N- ((1-methyl-1H-pyrazol-3- yl)methyl)picolinamide | 531 |
| Example 456 (A456) | 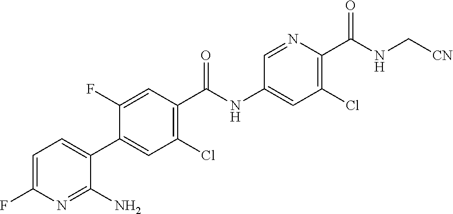 [SMILES: N#CCNC(=O)c1ncc(NC(=O)c2cc(F)c(-c3ccc(F)nc3N)cc2Cl)cc1Cl] | 5-(4-(2-amino-6- fluoropyridin-3-yl)-2-chloro- 5-fluorobenzamido)-3- chloro-N- (cyanomethyl)picolinamide | 523 |
| Example 457 (A457) | 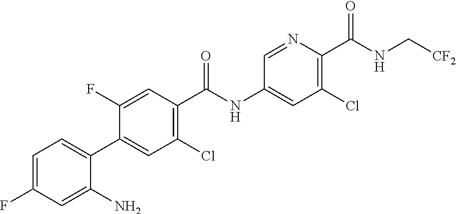 | 5-(2′-amino-5-chloro-2,4′- difluoro-[1,1′-biphenyl]-4- carboxamido)-3-chloro-N- (2,2-difluoro-2λ3- ethyl)picolinamide | 500 |
| Example 458 (A458) | 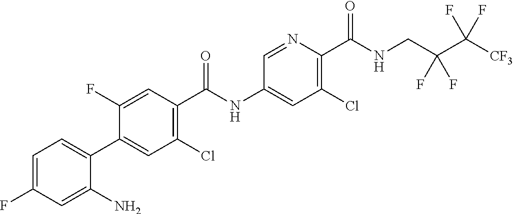 [SMILES: Nc1cc(F)ccc1-c1cc(Cl)c(C(=O)Nc2cnc(C(=O)NCC(F)(F)C(F)(F)C(F)(F)F)c(Cl)c2)cc1F] | 5-(2′-amino-5-chloro-2,4′- difluoro-[1,1′-biphenyl]-4- carboxamido)-3-chloro-N- (2,2,3,3,4,4,4- heptafluorobutyl) picolinamide | 619 |
| Example 459 (A459) | 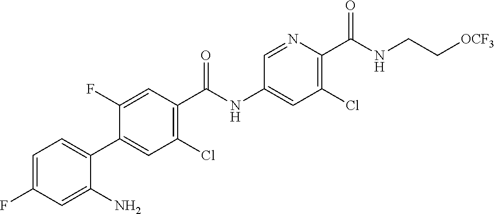 [SMILES: Nc1cc(F)ccc1-c1cc(Cl)c(C(=O)Nc2cnc(C(=O)NCCOC(F)(F)F)c(Cl)c2)cc1F] | 5-(2′-amino-5-chloro-2,4′- difluoro-[1,1′-biphenyl]-4- carboxamido)-3-chloro-N- (2-(trifluoromethoxy) ethyl)picolinamide | 549 |
| Example 460 (A460) | 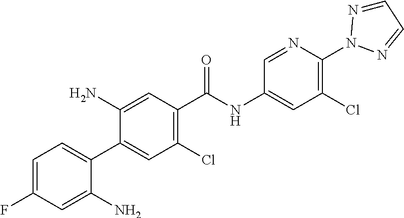 [SMILES: Nc1cc(F)ccc1-c1cc(Cl)c(C(=O)Nc2cnc(-n3nccn3)c(Cl)c2)cc1N] | 2,2′-diamino-5-chloro-N-(5- chloro-6-(2H-1,2,3-triazol-2- yl)pyridin-3-yl)-4′-fluoro- [1,1′-biphenyl]-4- carboxamide | 458 |
| Example 461 (A461) | 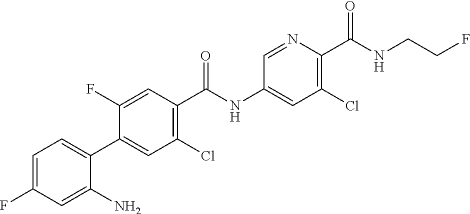 [SMILES: Nc1cc(F)ccc1-c1cc(Cl)c(C(=O)Nc2cnc(C(=O)NCCF)c(Cl)c2)cc1F] | 5-(2′-amino-5-chloro-2,4′- difluoro-[1,1′-biphenyl]-4- carboxamido)-3-chloro-N- (2-fluoroethyl)picolinamide | 483 |
| Example 462 (A462) | 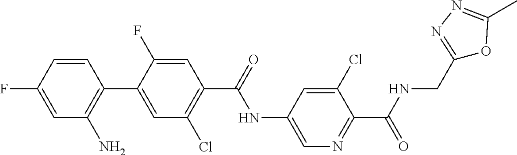 [SMILES: Cc1nnc(CNC(=O)c2ncc(NC(=O)c3cc(F)c(-c4ccc(F)cc4N)cc3Cl)cc2Cl)o1] | 5-(2′-amino-5-chloro-2,4′- difluoro-[1,1′-biphenyl]-4- carboxamido)-3-chloro-N- ((5-methyl-1,3,4-oxadiazol- 2-yl)methyl)picolinamide | 533 |
| Example 463 (A463) | 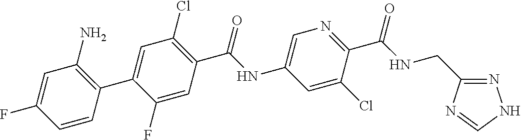 [SMILES: Nc1cc(F)ccc1-c1cc(Cl)c(C(=O)Nc2cnc(C(=O)NCc3nc[nH]n3)c(Cl)c2)cc1F] | N-((1H-1,2,4-triazol-3- yl)methyl)-5-(2′-amino-5- chloro-2,4′-difluoro-[1,1′- biphenyl]-4-carboxamido)-3- chloropicolinamide | 518 |
| Example 464 (A464) | 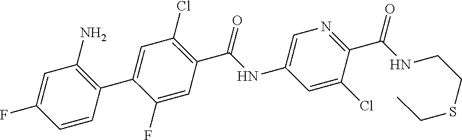 [SMILES: CCSCCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- (2-(ethylthio)ethyl) picolinamide | 525 |
| Example 465 (A465) | 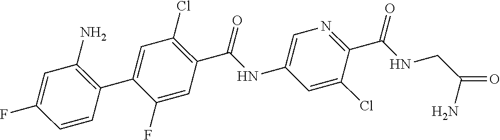 [SMILES: NC(=O)CNC(=O)c1ncc(NC(=O)c2cc(F)c(-c3ccc(F)cc3N)cc2Cl)cc1Cl] | N-(2-amino-2-oxoethyl)-5- (2′-amino-5-chloro-2,4′- difluoro-[1,1′-biphenyl]-4- carboxamido)-3- chloropicolinamide | 494 |
| Example 466 (A466) | 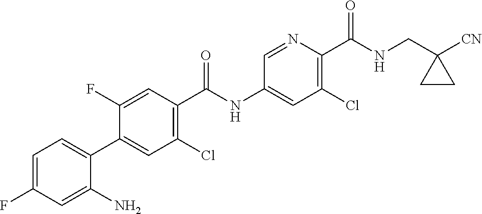 [SMILES: N#CC1(CNC(=O)c2ncc(NC(=O)c3cc(F)c(-c4ccc(F)cc4N)cc3Cl)cc2Cl)CC1] | 5-(2′-amino-5-chloro-2,4′- difluoro-[1,1′-biphenyl]-4- carboxamido)-3-chloro-N- ((1-cyanocyclopropyl) methyl)picolinamide | 516 |
| Example 467 (A467) | 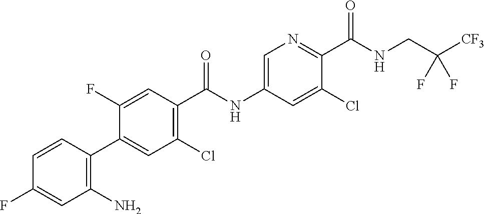 [SMILES: Nc1cc(F)ccc1-c1cc(Cl)c(C(=O)Nc2cnc(C(=O)NCC(F)(F)C(F)(F)F)c(Cl)c2)cc1F] | 5-(2′-amino-5-chloro-2,4′- difluoro-[1,1′-biphenyl]-4- carboxamido)-3-chloro-N- (2,2,3,3,3- pentafluoropropyl) picolinamide | 569 |
| Example 468 (A468) | 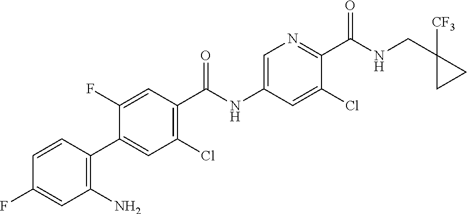 [SMILES: Nc1cc(F)ccc1-c1cc(Cl)c(C(=O)Nc2cnc(C(=O)NCC3(C(F)(F)F)CC3)c(Cl)c2)cc1F] | 5-(2′-amino-5-chloro-2,4′- difluoro-[1,1′-biphenyl]-4- carboxamido)-3-chloro-N- ((1-(trifluoromethyl) cyclopropyl)methyl) picolinamide | 559 |
| Example 469 (A469) | 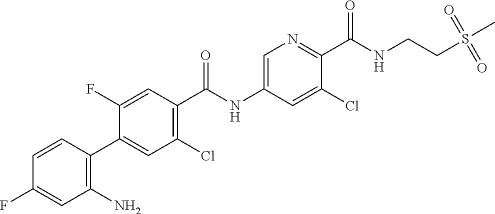 [SMILES: CS(=O)(=O)CCNC(=O)c1ncc(NC(=O)c2cc(F)c(-c3ccc(F)cc3N)cc2Cl)cc1Cl] | 5-(2′-amino-5-chloro-2,4′- difluoro-[1,1′-biphenyl]-4- carboxamido)-3-chloro-N- (2-(methylsulfonyl)ethyl) picolinamide | 543 |
| Example 470 (A470) | 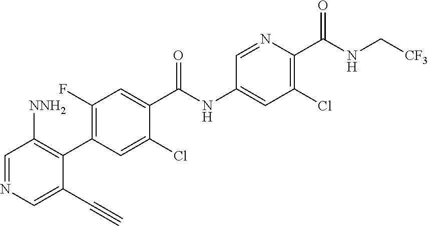 | 5-(4-(3-amino-5- ethynylpyridin-4-yl)-2- chloro-5-fluorobenzamido)- 3-chloro-N-(2,2,2- trifluoroethyl)picolinamide | 526 |
| Example 471 (A471) | 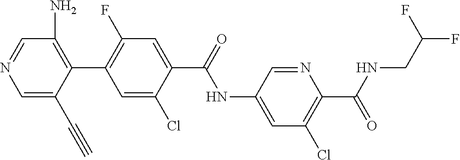 [SMILES: C#Cc1cncc(N)c1-c1cc(Cl)c(C(=O)Nc2cnc(C(=O)NCC(F)F)c(Cl)c2)cc1F] | 5-(4-(3-amino-5- ethynylpyridin-4-yl)-2- chloro-5-fluorobenzamido)- 3-chloro-N-(2,2- difluoroethyl)picolinamide | 508 |
| Example 472 (A472) | 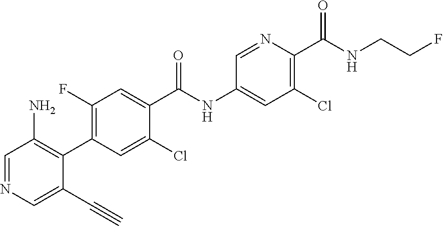 [SMILES: C#Cc1cncc(N)c1-c1cc(Cl)c(C(=O)Nc2cnc(C(=O)NCCF)c(Cl)c2)cc1F] | 5-(4-(3-amino-5- ethynylpyridin-4-yl)-2- chloro-5-fluorobenzamido)- 3-chloro-N-(2- fluoroethyl)picolinamide | 490 |
| Example 473 (A473) | 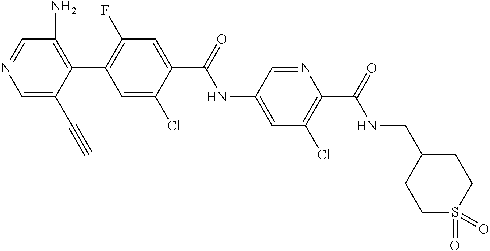 [SMILES: C#Cc1cncc(N)c1-c1cc(Cl)c(C(=O)Nc2cnc(C(=O)NCC3CCS(=O)(=O)CC3)c(Cl)c2)cc1F] | 5-(4-(3-amino-5- ethynylpyridin-4-yl)-2- chloro-5-fluorobenzamido)- 3-chloro-N-((1,1- dioxidotetrahydro-2H- thiopyran-4- yl)methyl)picolinamide | 590 |
| Example 474 (A474) | 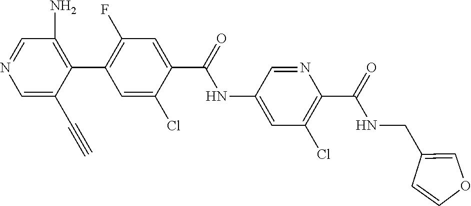 [SMILES: C#Cc1cncc(N)c1-c1cc(Cl)c(C(=O)Nc2cnc(C(=O)NCc3ccoc3)c(Cl)c2)cc1F] | 5-(4-(3-amino-5- ethynylpyridin-4-yl)-2- chloro-5-fluorobenzamido)- 3-chloro-N-(furan-3- ylmethyl)picolinamide | 524 |
| Example 475 (A475) | 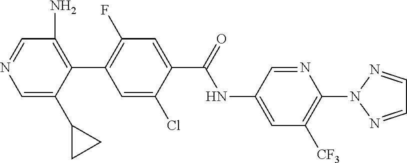 [SMILES: Nc1cncc(C2CC2)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5- cyclopropylpyridin-4-yl)-2- chloro-5-fluorobenzamide | 518 |
| Example 476 (A476) | 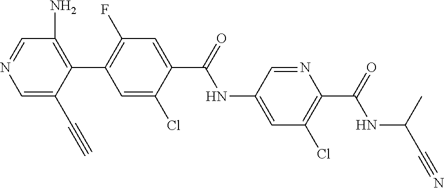 [SMILES: C#Cc1cncc(N)c1-c1cc(Cl)c(C(=O)Nc2cnc(C(=O)NC(C)C#N)c(Cl)c2)cc1F] | 5-(4-(3-amino-5- ethynylpyridin-4-yl)-2- chloro-5-fluorobenzamido)- 3-chloro-N-(1- cyanoethyl)picolinamide | 497 |
| Example 477 (A477) | 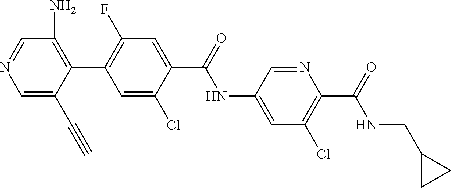 [SMILES: C#Cc1cncc(N)c1-c1cc(Cl)c(C(=O)Nc2cnc(C(=O)NCC3CC3)c(Cl)c2)cc1F] | 5-(4-(3-amino-5- ethynylpyridin-4-yl)-2- chloro-5-fluorobenzamido)- 3-chloro-N- (cyclopropylmethyl) picolinamide | 498 |
| Example 478 (A478) | 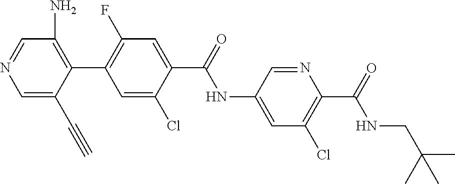 [SMILES: C#Cc1cncc(N)c1-c1cc(Cl)c(C(=O)Nc2cnc(C(=O)NCC(C)(C)C)c(Cl)c2)cc1F] | 5-(4-(3-amino-5- ethynylpyridin-4-yl)-2- chloro-5-fluorobenzamido)- 3-chloro-N- neopentylpicolinamide | 514 |
| Example 479 (A479) | 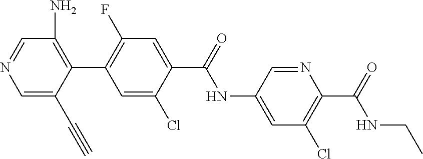 [SMILES: C#Cc1cncc(N)c1-c1cc(Cl)c(C(=O)Nc2cnc(C(=O)NCC)c(Cl)c2)cc1F] | 5-(4-(3-amino-5- ethynylpyridin-4-yl)-2- chloro-5-fluorobenzamido)- 3-chloro-N- ethylpicolinamide | 472 |
| Example 480 (A480) | 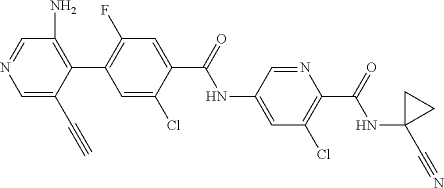 [SMILES: C#Cc1cncc(N)c1-c1cc(Cl)c(C(=O)Nc2cnc(C(=O)NC3(C#N)CC3)c(Cl)c2)cc1F] | 5-(4-(3-amino-5- ethynylpyridin-4-yl)-2- chloro-5-fluorobenzamido)- 3-chloro-N-(1- cyanocyclopropyl) picolinamide | 509 |
| Example 481 (A481) | 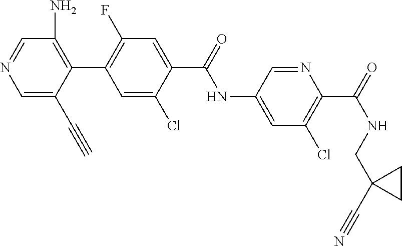 [SMILES: C#Cc1cncc(N)c1-c1cc(Cl)c(C(=O)Nc2cnc(C(=O)NCC3(C#N)CC3)c(Cl)c2)cc1F] | 5-(4-(3-amino-5- ethynylpyridin-4-yl)-2- chloro-5-fluorobenzamido)- 3-chloro-N-((1- cyanocyclopropyl)methyl) picolinamide | 523 |
| Example 482 (A482) | 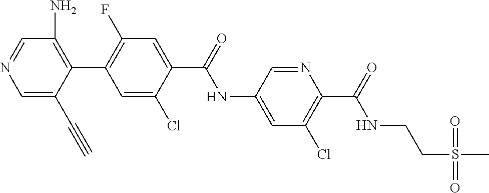 [SMILES: C#Cc1cncc(N)c1-c1cc(Cl)c(C(=O)Nc2cnc(C(=O)NCCS(C)(=O)=O)c(Cl)c2)cc1F] | 5-(4-(3-amino-5- ethynylpyridin-4-yl)-2- chloro-5-fluorobenzamido)- 3-chloro-N-(2- (methylsulfonyl)ethyl) picolinamide | 550 |
| Example 483 (A483) | 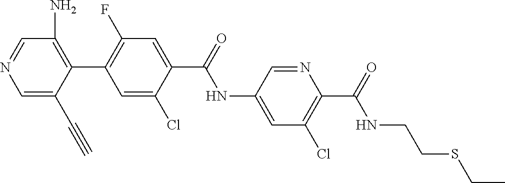 [SMILES: C#Cc1cncc(N)c1-c1cc(Cl)c(C(=O)Nc2cnc(C(=O)NCCSCC)c(Cl)c2)cc1F] | 5-(4-(3-amino-5- ethynylpyridin-4-yl)-2- chloro-5-fluorobenzamido)- 3-chloro-N-(2- (ethylthio)ethyl) picolinamide | 532 |
| Example 484 (A484) | 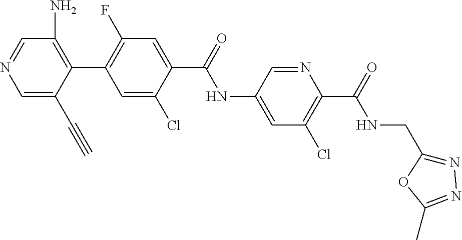 [SMILES: C#Cc1cncc(N)c1-c1cc(Cl)c(C(=O)Nc2cnc(C(=O)NCc3nnc(C)o3)c(Cl)c2)cc1F] | 5-(4-(3-amino-5- ethynylpyridin-4-yl)-2- chloro-5-fluorobenzamido)- 3-chloro-N-((5-methyl-1,3,4- oxadiazol-2- yl)methyl)picolinamide | 540 |
| Example 485 (A485) | 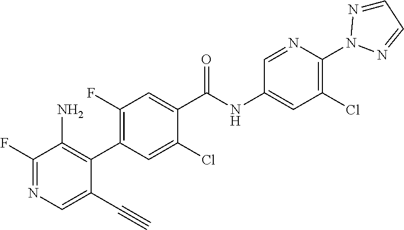 [SMILES: C#Cc1cnc(F)c(N)c1-c1cc(Cl)c(C(=O)Nc2cnc(-n3nccn3)c(Cl)c2)cc1F] | 4-(3-amino-5-ethynyl-2- fluoropyridin-4-yl)-2-chloro- N-(5-chloro-6-(2H-1,2,3- triazol-2-yl)pyridin-3-yl)-5- fluorobenzamide | 486 |
| Example 486 (A486) | 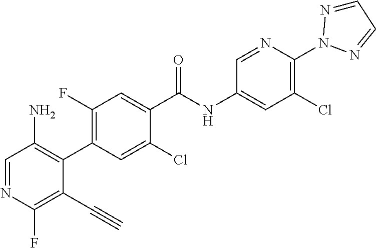 [SMILES: C#Cc1c(F)ncc(N)c1-c1cc(Cl)c(C(=O)Nc2cnc(-n3nccn3)c(Cl)c2)cc1F] | 4-(5-amino-3-ethynyl-2- fluoropyridin-4-yl)-2-chloro- N-(5-chloro-6-(2H-1,2,3- triazol-2-yl)pyridin-3-yl)-5- fluorobenzamide | 486 |
| Example 487 (A487) | 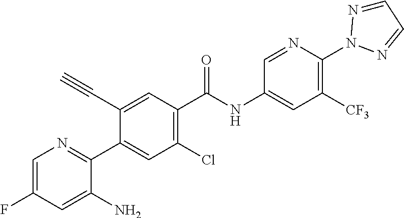 [SMILES: C#Cc1cc(C(=O)Nc2cnc(-n3nccn3)c(C(F)(F)F)c2)c(Cl)cc1-c1ncc(F)cc1N] | N-(6-(2H-1,2,3-triazol-2-yl)- 5-(trifluoromethyl)pyridin-3- yl)-4-(3-amino-5- fluoropyridin-2-yl)-2-chloro- 5-ethynylbenzamide | 502 |
| Example 488 (A488) | 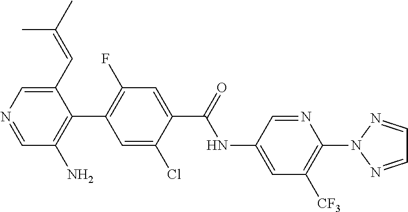 [SMILES: CC(C)=Cc1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(2- methylprop-1-en-1- yl)pyridin-4-yl)-2-chloro-5- fluorobenzamide | 532 |
| Example 489 (A489) | 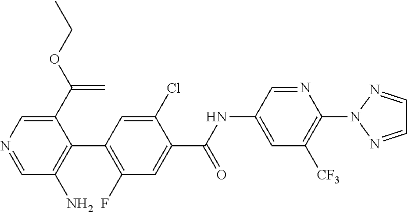 [SMILES: C=C(OCC)c1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(1- ethoxyvinyl)pyridin-4-yl)-2- chloro-5-fluorobenzamide | 548 |
| Example 490 (A490) | 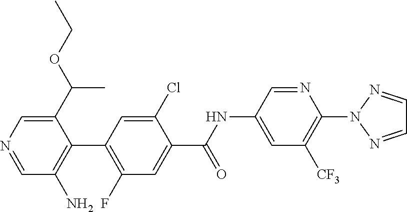 [SMILES: CCOC(C)c1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(1- ethoxyethyl)pyridin-4-yl)-2- chloro-5-fluorobenzamide | 550 |
| Example 491 (A491) | 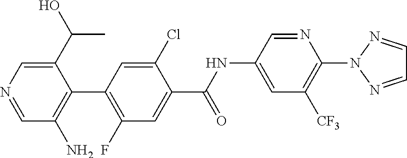 [SMILES: CC(O)c1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(1- hydroxyethyl)pyridin-4-yl)- 2-chloro-5-fluorobenzamide | 522 |
| Example 492 (A492) | 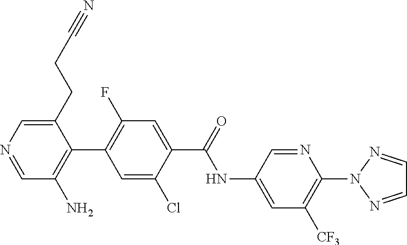 [SMILES: N#CCCc1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(2- cyanoethyl)pyridin-4-yl)-2- chloro-5-fluorobenzamide | 531 |
| Example 493 (A493) | 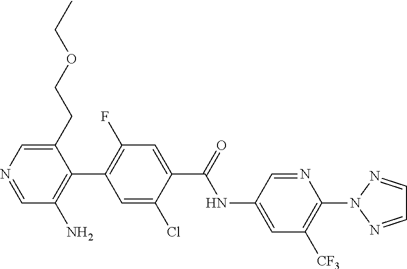 [SMILES: CCOCCc1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(2- ethoxyethyl)pyridin-4-yl)-2- chloro-5-fluorobenzamide | 550 |
| Example 494 (A494) | 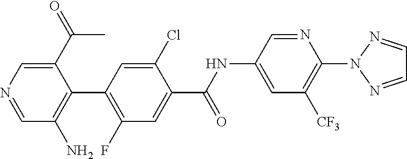 [SMILES: CC(=O)c1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-acetyl-5- aminopyridin-4-yl)-2-chloro- 5-fluorobenzamide | 520 |
| Example 495 (A495) | 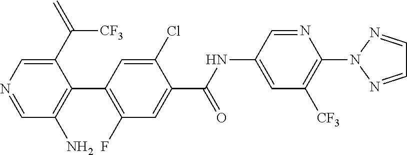 [SMILES: C=C(c1cncc(N)c1-c1cc(Cl)c(C(=O)Nc2cnc(-n3nccn3)c(C(F)(F)F)c2)cc1F)C(F)(F)F] | N-(6-(2H-1,2,3-triazol-2-yl)- 5-(trifluoromethyl)pyridin-3- yl)-4-(3-amino-5-(3,3,3- trifluoroprop-1-en-2- yl)pyridin-4-yl)-2-chloro-5- fluorobenzamide | 572 |
| Example 496 (A496) | 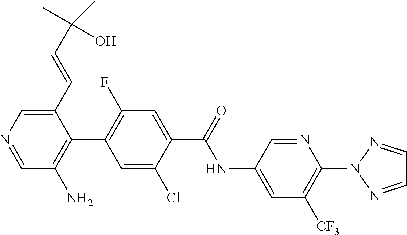 [SMILES: CC(C)(O)/C=C/c1cncc(N)c1-c1cc(Cl)c(C(=O)Nc2cnc(-n3nccn3)c(C(F)(F)F)c2)cc1F] | (E)-N-(6-(2H-1,2,3-triazol- 2-yl)-5- (trifluoromethyl)pyridin-3- yl)-4-(3-amino-5-(3- hydroxy-3-methylbut-1-en- 1-yl)pyridin-4-yl)-2-chloro- 5-fluorobenzamide | 562 |
| Example 497 (A497) | 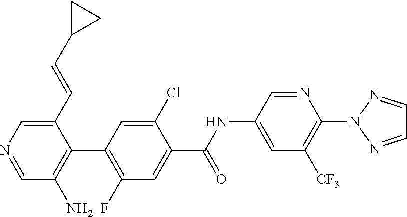 [SMILES: Nc1cncc(/C=C/C2CC2)c1-c1cc(Cl)c(C(=O)Nc2cnc(-n3nccn3)c(C(F)(F)F)c2)cc1F] | (E)-N-(6-(2H-1,2,3-triazol- 2-yl)-5- (trifluoromethyl)pyridin-3- yl)-4-(3-amino-5-(2- cyclopropylvinyl)pyridin-4- yl)-2-chloro-5- fluorobenzamide | 544 |
| Example 498 (A498) | 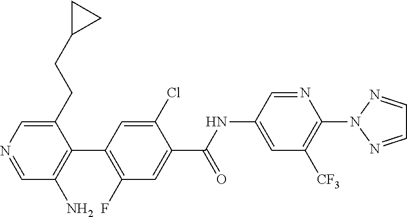 [SMILES: Nc1cncc(CCC2CC2)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(2- cyclopropylethyl)pyridin-4- yl)-2-chloro-5- fluorobenzamide | 546 |
| Example 499 (A499) | 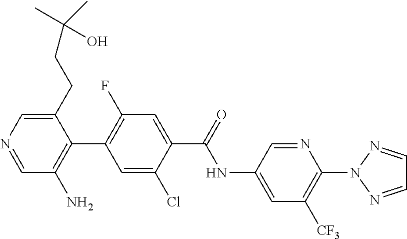 [SMILES: CC(C)(O)CCc1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(3- hydroxy-3- methylbutyl)pyridin-4-yl)-2- chloro-5-fluorobenzamide | 564 |
| Example 500 (A500) | 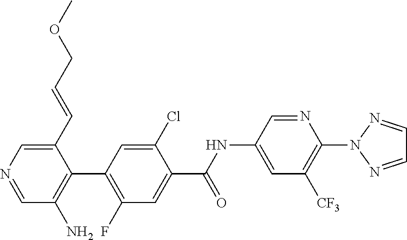 [SMILES: COC/C=C/c1cncc(N)c1-c1cc(Cl)c(C(=O)Nc2cnc(-n3nccn3)c(C(F)(F)F)c2)cc1F] | (E)-N-(6-(2H-1,2,3-triazol- 2-yl)-5- (trifluoromethyl)pyridin-3- yl)-4-(3-amino-5-(3- methoxyprop-1-en-1- yl)pyridin-4-yl)-2-chloro-5- fluorobenzamide | 548 |
| Example 501 (A501) | 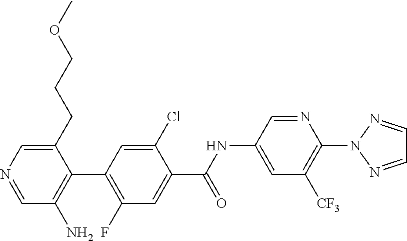 [SMILES: COCCCc1cncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(3- methoxypropyl)pyridin-4- yl)-2-chloro-5- fluorobenzamide | 550 |
| Example 502 (A502) | 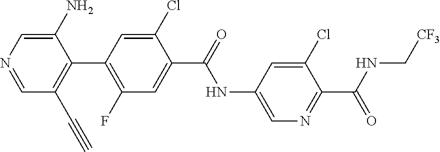 [SMILES: C#Cc1cncc(N)c1-c1cc(Cl)c(C(=O)Nc2cnc(C(=O)NCC(F)(F)F)c(Cl)c2)cc1F] | 5-(4-(3-amino-5- ethynylpyridin-4-yl)-2- chloro-5-fluorobenzamido)- 3-chloro-N-(2,2,2- trifluoroethyl)picolinamide | 526 |
| Example 503 (A503) | 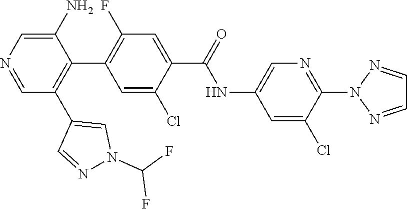 [SMILES: Nc1cncc(-c2cnn(C(F)F)c2)c1-c1cc(Cl)c(C(=O)Nc2cnc(-n3nccn3)c(Cl)c2)cc1F] | 4-(3-amino-5-(1- (difluoromethyl)-1H- pyrazol-4-yl)pyridin-4-yl)-2- chloro-N-(5-chloro-6-(2H- 1,2,3-triazol-2-yl)pyridin-3- yl)-5-fluorobenzamide | 560 |
| Example 504 (A504) | 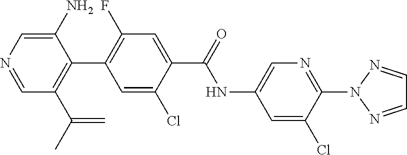 [SMILES: C=C(C)c1cncc(N)c1-c1cc(Cl)c(C(=O)Nc2cnc(-n3nccn3)c(Cl)c2)cc1F] | 4-(3-amino-5-(prop-1-en-2- yl)pyridin-4-yl)-2-chloro-N- (5-chloro-6-(2H-1,2,3- triazol-2-yl)pyridin-3-yl)-5- fluorobenzamide | 484 |
| Example 505 (A505) | 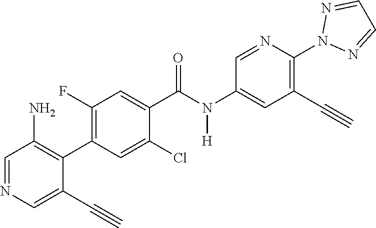 [SMILES: C#Cc1cc(NC(=O)c2cc(F)c(-c3c(N)cncc3C#C)cc2Cl)cnc1-n1nccn1] | 4-(3-amino-5- ethynylpyridin-4-yl)-2- chloro-N-(5-ethynyl-6-(2H- 1,2,3-triazol-2-yl)pyridin-2- yl)-5-fluorobenzamide | 458 |
| Example 506 (A506) | 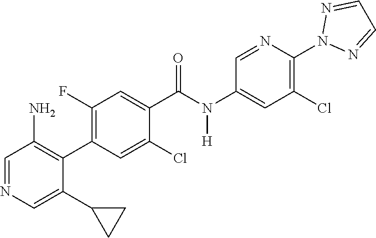 [SMILES: Nc1cncc(C2CC2)c1-c1cc(Cl)c(C(=O)Nc2cnc(-n3nccn3)c(Cl)c2)cc1F] | 4-(3-amino-5- cyclopropylpyridin-4-yl)-2- chloro-N-(5-chloro-6-(2H- 1,2,3-triazol-2-yl)pyridin-3- yl)-5-fluorobenzamide | 484 |
| Example 507 (A507) | 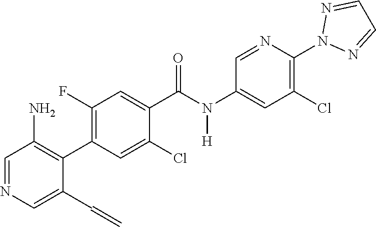 [SMILES: C=Cc1cncc(N)c1-c1cc(Cl)c(C(=O)Nc2cnc(-n3nccn3)c(Cl)c2)cc1F] | 4-(3-amino-5-vinylpyridin- 4-yl)-2-chloro-N-(5-chloro- 6-(2H-1,2,3-triazol-2- yl)pyridin-3-yl)-5- fluorobenzamide | 470 |
| Example 508 (A508) | 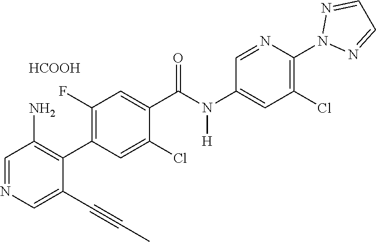 [SMILES: CC#Cc1cncc(N)c1-c1cc(Cl)c(C(=O)Nc2cnc(-n3nccn3)c(Cl)c2)cc1F ; "HCOOH"] | 4-(3-amino-5-(prop-1-yn-1- yl)pyridin-4-yl)-2-chloro-N- (5-chloro-6-(2H-1,2,3- triazol-2-yl)pyridin-3-yl)-5- fluorobenzamide formate | 528 |
| Example 509 (A509) | 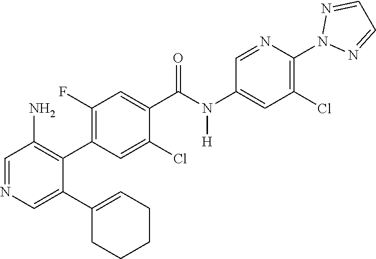 [SMILES: Nc1cncc(C2=CCCCC2)c1-c1cc(Cl)c(C(=O)Nc2cnc(-n3nccn3)c(Cl)c2)cc1F] | 4-(3-amino-5-(cyclohex-1- en-1-yl)pyridin-4-yl)-2- chloro-N-(5-chloro-6-(2H- 1,2,3-triazol-2-yl)pyridin-3- yl)-5-fluorobenzamide | 524 |
| Example 512 (A512) | 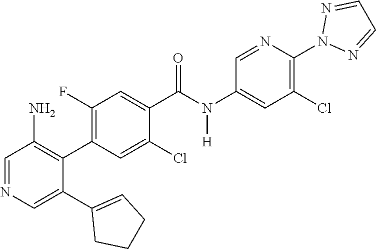 [SMILES: Nc1cncc(C2=CCCC2)c1-c1cc(Cl)c(C(=O)Nc2cnc(-n3nccn3)c(Cl)c2)cc1F] | 4-(3-amino-5-(cyclopent-1- en-1-yl)pyridin-4-yl)-2- chloro-N-(5-chloro-6-(2H- 1,2,3-triazol-2-yl)pyridin-3- yl)-5-fluorobenzamide | 556 |
| Example 513 (A513) | 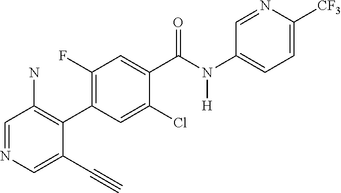 | 4-(3-amino-5- ethynylpyridin-4-yl)-2- chloro-5-fluoro-N-(6- (trifluoromethyl)pyridin-3- yl)benzamide | 435 |
| Example 514 (A514) | 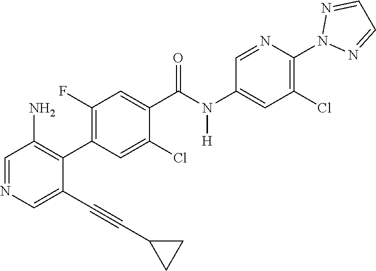 [SMILES: Nc1cncc(C#CC2CC2)c1-c1cc(Cl)c(C(=O)Nc2cnc(-n3nccn3)c(Cl)c2)cc1F] | 4-(3-amino-5- (cyclopropylethynyl)pyridin- 4-yl)-2-chloro-N-(5-chloro- 6-(2H-1,2,3-triazol-2- yl)pyridin-3-yl)-5- fluorobenzamide | 508 |
| Example 515 (A515) | 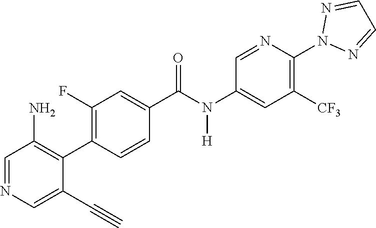 [SMILES: C#Cc1cncc(N)c1-c1ccc(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5- ethynylpyridin-4-yl)-3- fluorobenzamide | 467 |
| Example 516 (A516) | 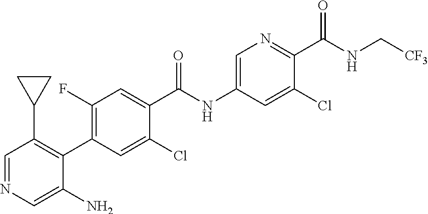 [SMILES: Nc1cncc(C2CC2)c1-c1cc(Cl)c(C(=O)Nc2cnc(C(=O)NCC(F)(F)F)c(Cl)c2)cc1F] | 5-(4-(3-amino-5- cyclopropylpyridin-4-yl)-2- chloro-5-fluorobenzamido)- 3-chloro-N-(2,2,2- trifluoroethyl)picolinamide | 542 |
| Example 517 (A517) | 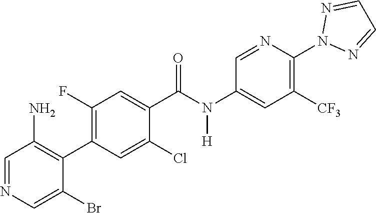 [SMILES: Nc1cncc(Br)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5- bromopyridin-4-yl)-2- chloro-5-fluorobenzamide | 603 |
| Example 518 (A518) | 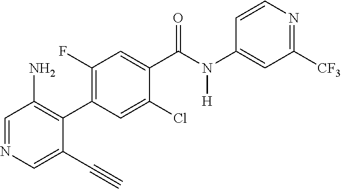 [SMILES: C#Cc1cncc(N)c1-c1cc(Cl)c(C(=O)Nc2ccnc(C(F)(F)F)c2)cc1F] | 4-(3-amino-5- ethynylpyridin-4-yl)-2- chloro-5-fluoro-N-(2- (trifluoromethyl)pyridin-4- yl)benzamide | 481 |
| Example 519 (A519) | 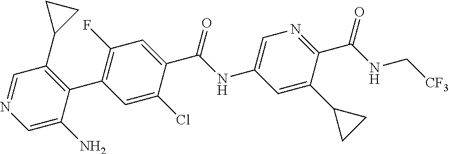 [SMILES: Nc1cncc(C2CC2)c1-c1cc(Cl)c(C(=O)Nc2cnc(C(=O)NCC(F)(F)F)c(C3CC3)c2)cc1F] | 5-(4-(3-amino-5- cyclopropylpyridin-4-yl)-2- chloro-5-fluorobenzamido)- 3-cyclopropyl-N-(2,2,2- trifluoroethyl)picolinamide | 548 |
| Example 521 (A521) | 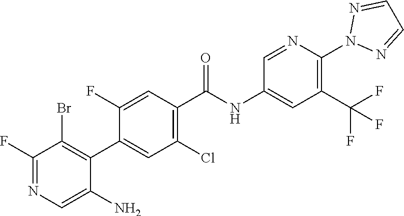 [SMILES: Nc1cnc(F)c(Br)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(5-amino-3-bromo-2- fluoropyridin-4-yl)-2-chloro- 5-fluorobenzamide | 575 |
| Example 522 (A522) | 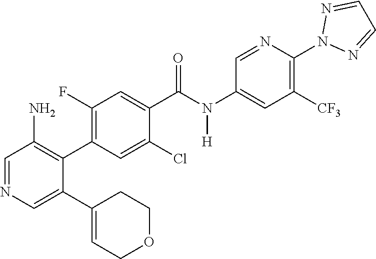 [SMILES: Nc1cncc(C2=CCOCC2)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(3,6- dihydro-2H-pyran-4- yl)pyridin-4-yl)-2-chloro-5- fluorobenzamide | 596 |
| Example 523 (A523) | 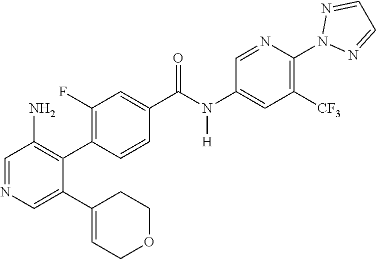 [SMILES: Nc1cncc(C2=CCOCC2)c1-c1ccc(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(3,6- dihydro-2H-pyran-4- yl)pyridin-4-yl)-3- fluorobenzamide | 562 |
| Example 524 (A524) | 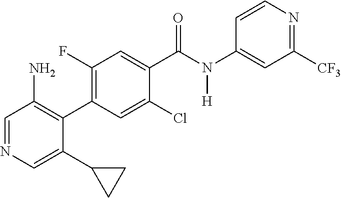 [SMILES: Nc1cncc(C2CC2)c1-c1cc(Cl)c(C(=O)Nc2ccnc(C(F)(F)F)c2)cc1F] | 4-(3-amino-5- cyclopropylpyridin-4-yl)-2- chloro-5-fluoro-N-(2- (trifluoromethyl)pyridin-4- yl)benzamide | 451 |
| Example 525 (A525) | 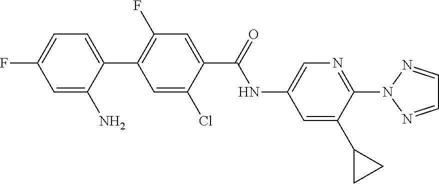 [SMILES: Nc1cc(F)ccc1-c1cc(Cl)c(C(=O)Nc2cnc(-n3nccn3)c(C3CC3)c2)cc1F] | 2′-amino-5-chloro-N-(5- cyclopropyl-6-(2H-1,2,3- triazol-2-yl)pyridin-3-yl)- 2,4′-difluoro-[1,1′-biphenyl]- 4-carboxamide | 467 |
| Example 526 (A526) | 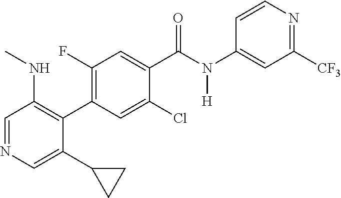 [SMILES: CNc1cncc(C2CC2)c1-c1cc(Cl)c(C(=O)Nc2ccnc(C(F)(F)F)c2)cc1F] | 2-chloro-4-(3-cyclopropyl-5- (methylamino)pyridin-4-yl)- 5-fluoro-N-(2- (trifluoromethyl)pyridin-4- yl)benzamide | 465 |
| Example 527 (A527) | 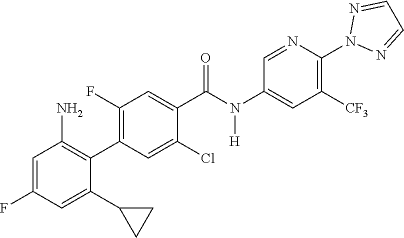 [SMILES: Nc1cc(F)cc(C2CC2)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-2′-amino-5-chloro-6′- cyclopropyl-2,4′-difluoro- [1,1′-biphenyl]-4- carboxamide | 535 |
| Example 528 (A528) | 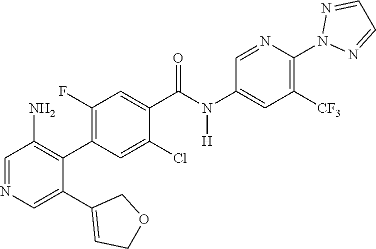 [SMILES: Nc1cncc(C2=CCOC2)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(2,5- dihydrofuran-3-yl)pyridin-4- yl)-2-chloro-5- fluorobenzamide | 546 |
| Example 529 (A529) | 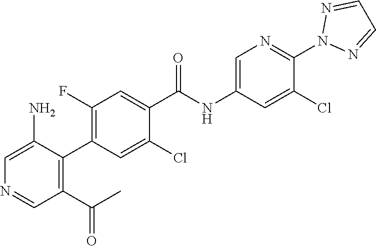 [SMILES: CC(=O)c1cncc(N)c1-c1cc(Cl)c(C(=O)Nc2cnc(-n3nccn3)c(Cl)c2)cc1F] | 4-(3-acetyl-5-aminopyridin- 4-yl)-2-chloro-N-(5-chloro- 6-(2H-1,2,3-triazol-2- yl)pyridin-3-yl)-5- fluorobenzamide | 486 |
| Example 530 (A530) | 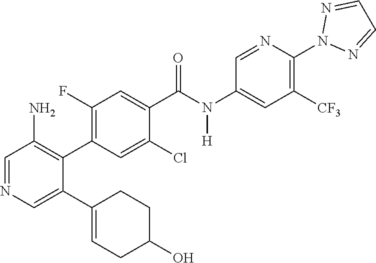 [SMILES: Nc1cncc(C2=CCC(O)CC2)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-(4- hydroxycyclohex-1-en-1- yl)pyridin-4-yl)-2-chloro-5- fluorobenzamide | 574 |
| Example 531 (A531) | 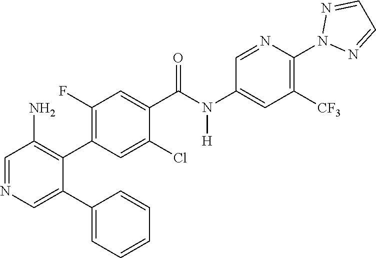 [SMILES: Nc1cncc(-c2ccccc2)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5- phenylpyridin-4-yl)-2- chloro-5-fluorobenzamide | 554 |
| Example 532 (A532) | 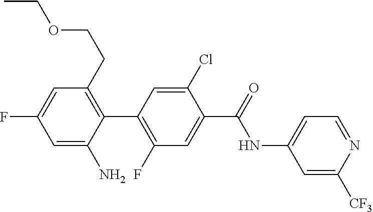 [SMILES: CCOCCc1cc(F)cc(N)c1-c1cc(Cl)c(C(=O)Nc2ccnc(C(F)(F)F)c2)cc1F] | 2′-amino-5-chloro-6′-(2- ethoxyethyl)-2,4′-difluoro- N-(2- (trifluoromethyl)pyridin-4- yl)-[1,1′-biphenyl]-4- carboxamide | 500 |
| Example 533 (A533) | 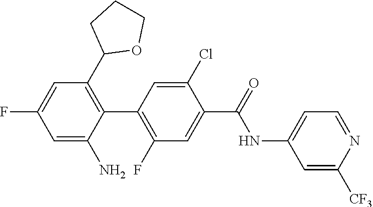 [SMILES: Nc1cc(F)cc(C2CCCO2)c1-c1cc(Cl)c(C(=O)Nc2ccnc(C(F)(F)F)c2)cc1F] | 2′-amino-5-chloro-2,4′- difluoro-6′-(tetrahydrofuran- 2-yl)-N-(2- (trifluoromethyl)pyridin-4- yl)-[1,1′-biphenyl]-4- carboxamide | 498 |
| Example 534 (A534) | 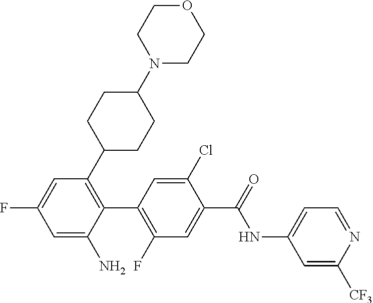 [SMILES: Nc1cc(F)cc(C2CCC(N3CCOCC3)CC2)c1-c1cc(Cl)c(C(=O)Nc2ccnc(C(F)(F)F)c2)cc1F] | 2′-amino-5-chloro-2,4′- difluoro-6′-(4- morpholinocyclohexyl)-N- (2-(trifluoromethyl)pyridin- 4-yl)-[1,1′-biphenyl]-4- carboxamide | 595 |
| Example 535 (A535) | 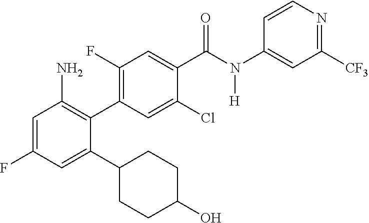 [SMILES: Nc1cc(F)cc(C2CCC(O)CC2)c1-c1cc(Cl)c(C(=O)Nc2ccnc(C(F)(F)F)c2)cc1F] | 2′-amino-5-chloro-2,4′- difluoro-6′-(4- hydroxycyclohexyl)-N-(2- (trifluoromethyl)pyridin-4- yl)-[1,1′-biphenyl]-4- carboxamide | 526 |
| Example 536 (A536) | 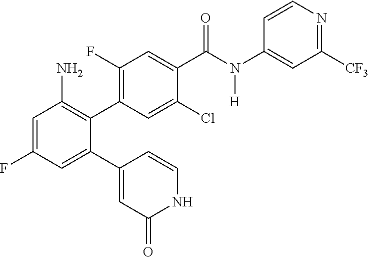 [SMILES: Nc1cc(F)cc(-c2cc[nH]c(=O)c2)c1-c1cc(Cl)c(C(=O)Nc2ccnc(C(F)(F)F)c2)cc1F] | 2′-amino-5-chloro-2,4′- difluoro-6′-(2-oxo-1,2- dihydropyridin-4-yl)-N-(2- (trifluoromethyl)pyridin-4- yl)-[1,1′-biphenyl]-4- carboxamide | 521 |
| Example 537 (A537) | 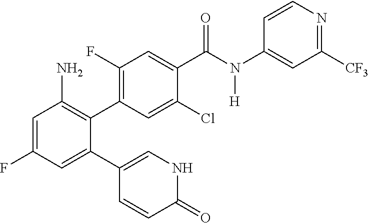 [SMILES: Nc1cc(F)cc(-c2ccc(=O)[nH]c2)c1-c1cc(Cl)c(C(=O)Nc2ccnc(C(F)(F)F)c2)cc1F] | 2′-amino-5-chloro-2,4′- difluoro-6′-(6-oxo-1,6- dihydropyridin-3-yl)-N-(2- (trifluoromethyl)pyridin-4- yl)-[1,1′-biphenyl]-4- carboxamide | 521 |
| Example 538 (A538) | 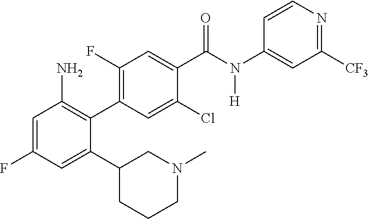 [SMILES: CN1CCCC(c2cc(F)cc(N)c2-c2cc(Cl)c(C(=O)Nc3ccnc(C(F)(F)F)c3)cc2F)C1] | 2′-amino-5-chloro-2,4′- difluoro-6′-(1- methylpyridin-3-yl)-N-(2- (trifluoromethyl)pyridin-4- yl)-[1,1′-biphenyl]-4- carboxamide | 525 |
| Example 539 (A539) | 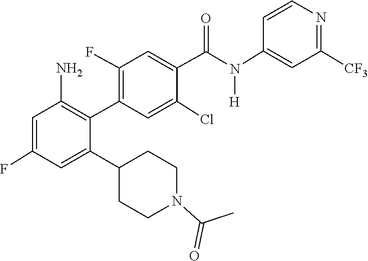 [SMILES: CC(=O)N1CCC(c2cc(F)cc(N)c2-c2cc(Cl)c(C(=O)Nc3ccnc(C(F)(F)F)c3)cc2F)CC1] | 2′-(1-acetylpiperidin-4-yl)- 6′-amino-5-chloro-2,4′- difluoro-N-(2- (trifluoromethyl)pyridin-4- yl)-[1,1′-biphenyl]-4- carboxamide | 553 |
| Example 540 (A540) | 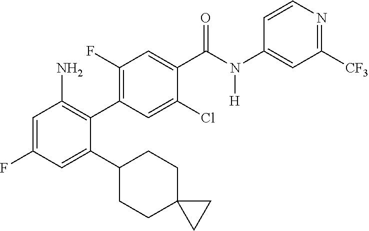 [SMILES: Nc1cc(F)cc(C2CCC3(CC2)CC3)c1-c1cc(Cl)c(C(=O)Nc2ccnc(C(F)(F)F)c2)cc1F] | 2′-amino-5-chloro-2,4′- difluoro-6′-(spiro[2.5]octan- 6-yl)-N-(2- (trifluoromethyl)pyridin-4- yl)-[1,1′-biphenyl]-4- carboxamide | 536 |
| Example 541 (A541) | 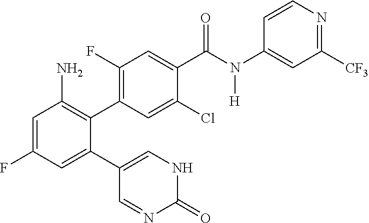 [SMILES: Nc1cc(F)cc(-c2cnc(=O)[nH]c2)c1-c1cc(Cl)c(C(=O)Nc2ccnc(C(F)(F)F)c2)cc1F] | 2′-amino-5-chloro-2,4′- difluoro-6′-(2-oxo-1,2- dihydropyrimidin-5-yl)-N- (2-(trifluoromethyl)pyridin- 4-yl)-[1,1′-biphenyl]-4- carboxamide | 522 |
| Example 542 (A542) | 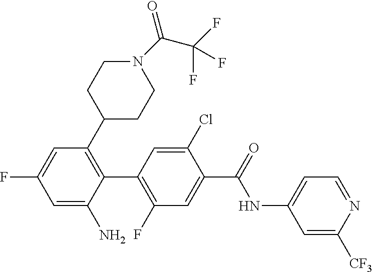 [SMILES: Nc1cc(F)cc(C2CCN(C(=O)C(F)(F)F)CC2)c1-c1cc(Cl)c(C(=O)Nc2ccnc(C(F)(F)F)c2)cc1F] | 2′-amino-5-chloro-2,4′- difluoro-6′-(1-(2,2,2- trifluoroacetyl)piperidin-4- yl)-N-(2- (trifluoromethyl)pyridin-4- yl)-[1,1′-biphenyl]-4- carboxamide | 607 |
| Example 543 (A543) | 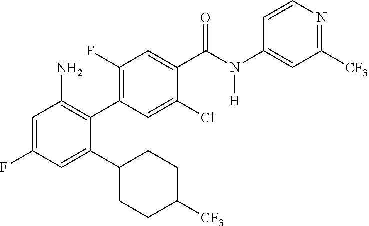 [SMILES: Nc1cc(F)cc(C2CCC(C(F)(F)F)CC2)c1-c1cc(Cl)c(C(=O)Nc2ccnc(C(F)(F)F)c2)cc1F] | 2′-amino-5-chloro-2,4′- difluoro-6′-(4- (trifluoromethyl)cyclo- hexyl)-N-(2- (trifluoromethyl)pyridin-4- yl)-[1,1′-biphenyl]-4- carboxamide | 578 |
| Example 544 (A544) | 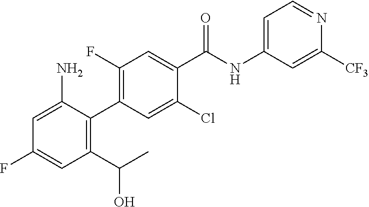 [SMILES: CC(O)c1cc(F)cc(N)c1-c1cc(Cl)c(C(=O)Nc2ccnc(C(F)(F)F)c2)cc1F] | 2′-amino-5-chloro-2,4′- difluoro-6′-(1- hydroxyethyl)-N-(2- (trifluoromethyl)pyridin-4- yl)-[1,1′-biphenyl]-4- carboxamide | 472 |
| Example 545 (A545) | 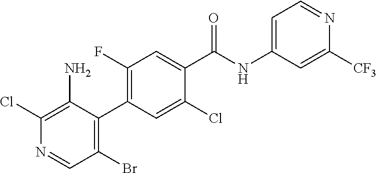 [SMILES: Nc1c(Cl)ncc(Br)c1-c1cc(Cl)c(C(=O)Nc2ccnc(C(F)(F)F)c2)cc1F] | 4-(3-amino-5-bromo-2- chloropyridin-4-yl)-2- chloro-5-fluoro-N-(2- (trifluoromethyl)pyridin-4- yl)benzamide | 524 |
| Example 546 (A546) | 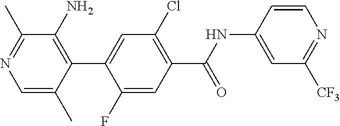 [SMILES: Cc1cnc(C)c(N)c1-c1cc(Cl)c(C(=O)Nc2ccnc(C(F)(F)F)c2)cc1F] | 4-(3-amino-2,5- dimethylpyridin-4-yl)-2- chloro-5-fluoro-N-(2- (trifluoromethyl)pyridin-4- yl)benzamide | 439 |
| Example 547 (A547) | 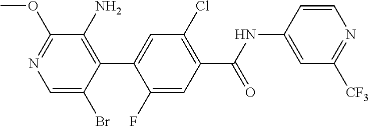 [SMILES: COc1ncc(Br)c(-c2cc(Cl)c(C(=O)Nc3ccnc(C(F)(F)F)c3)cc2F)c1N] | 4-(3-amino-5-bromo-2- methoxypyridin-4-yl)-2- chloro-5-fluoro-N-(2- (trifluoromethyl)pyridin-4- yl)benzamide | 520 |
| Example 548 (A548) | 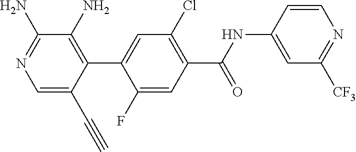 [SMILES: C#Cc1cnc(N)c(N)c1-c1cc(Cl)c(C(=O)Nc2ccnc(C(F)(F)F)c2)cc1F] | 2-chloro-4-(2,3-diamino-5- ethynylpyridin-4-yl)-5- fluoro-N-(2- (trifluoromethyl)pyridin-4- yl)benzamide | 450 |
| Example 549 (A549) | 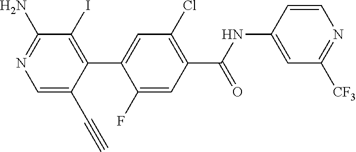 [SMILES: C#Cc1cnc(N)c(I)c1-c1cc(Cl)c(C(=O)Nc2ccnc(C(F)(F)F)c2)cc1F] | 4-(2-amino-5-ethynyl-3- iodopyridin-4-yl)-2-chloro- 5-fluoro-N-(2- (trifluoromethyl)pyridin-4- yl)benzamide | 561 |
| Example 550 (A550) | 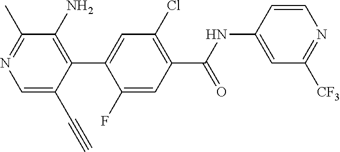 [SMILES: C#Cc1cnc(C)c(N)c1-c1cc(Cl)c(C(=O)Nc2ccnc(C(F)(F)F)c2)cc1F] | 4-(3-amino-5-ethynyl-2- methylpyridin-4-yl)-2- chloro-5-fluoro-N-(2- (trifluoromethyl)pyridin-4- yl)benzamide | 449 |
| Example 551 (A551) | 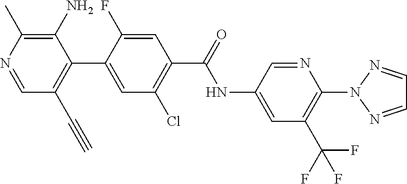 [SMILES: C#Cc1cnc(C)c(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-ethynyl-2- methylpyridin-4-yl)-2- chloro-5-fluorobenzamide | 516 |
| Example 552 (A552) | 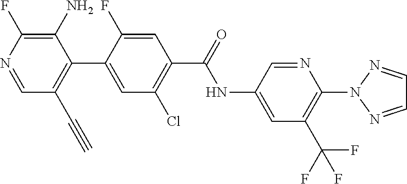 [SMILES: C#Cc1cnc(F)c(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-ethynyl-2- fluoropyridin-4-yl)-2-chloro- 5-fluorobenzamide | 520 |
| Example 554 (A554) | 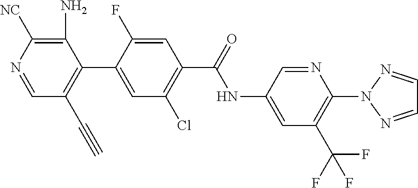 [SMILES: C#Cc1cnc(C#N)c(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-2-cyano-5- ethynylpyridin-4-yl)-2- chloro-5-fluorobenzamide | 527 |
| Example 556 (A556) | 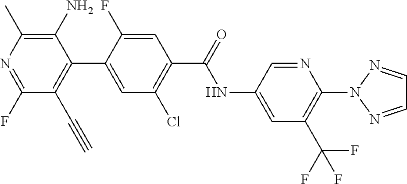 [SMILES: C#Cc1c(F)nc(C)c(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-ethynyl-6- fluoro-2-methylpyridin-4- yl)-2-chloro-5- fluorobenzamide | 534 |
| Example 557 (A557) | 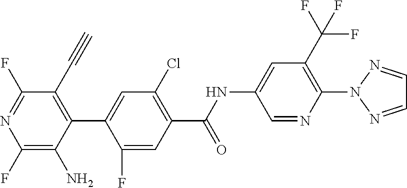 [SMILES: C#Cc1c(F)nc(F)c(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5-ethynyl- 2,6-difluoropyridin-4-yl)-2- chloro-5-fluorobenzamide | 538 |
| Example 561 (A561) | 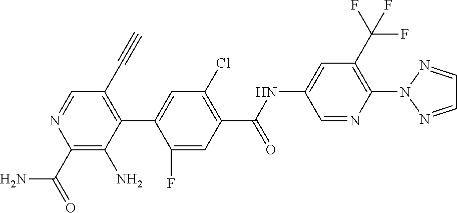 [SMILES: C#Cc1cnc(C(N)=O)c(N)c1-c1cc(Cl)c(C(=O)Nc2cnc(-n3nccn3)c(C(F)(F)F)c2)cc1F] | 4-(4-((6-(2H-1,2,3-triazol-2- yl)-5- (trifluoromethyl)pyridin-3- yl)carbamoyl)-5-chloro-2- fluorophenyl)-3-amino-5- ethynylpicolinamide | 545 |
| Example 562 (A562) | 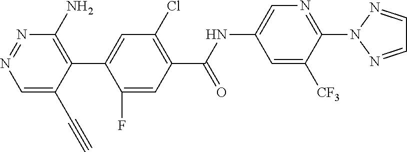 [SMILES: C#Cc1cnnc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(3-amino-5- ethynylpyridazin-4-yl)-2- chloro-5-fluorobenzamide | 503 |
| Example 563 (A563) | 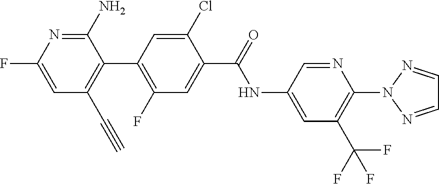 [SMILES: C#Cc1cc(F)nc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(2-amino-4-ethynyl-6- fluoropyridin-3-yl)-2-chloro- 5-fluorobenzamide | 520 |
| Example 564 (A564) | 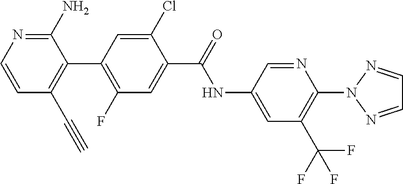 [SMILES: C#Cc1ccnc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(2-amino-4- ethynylpyridin-3-yl)-2- chloro-5-fluorobenzamide | 502 |
| Example 565 (A565) | 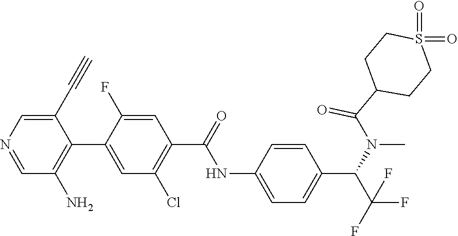 [SMILES: C#Cc1cncc(N)c1-c1cc(Cl)c(C(=O)Nc2ccc([C@H](N(C)C(=O)C3CCS(=O)(=O)CC3)C(F)(F)F)cc2)cc1F] | N-((1S)-1-(4-(4-(3-amino-5- ethynylpyridin-4-yl)-2- chloro-5- fluorobenzamido)phenyl)- 2,2,2-trifluoroethyl)-N- methyltetrahydro2H- thiopyran-4- carboxamide 1,1-dioxide | 637 |
| Example 566 (A566) | 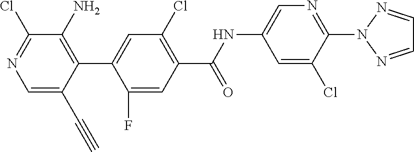 [SMILES: C#Cc1cnc(Cl)c(N)c1-c1cc(Cl)c(C(=O)Nc2cnc(-n3nccn3)c(Cl)c2)cc1F] | 4-(3-amino-2-chloro-5- ethynylpyridin-4-yl)-2- chloro-N-(5-chloro-6-(2H- 1,2,3-triazol-2-yl)pyridin-3- yl)-5-fluorobenzamide | 502 |
| Example 567 (A567) | 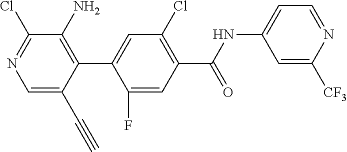 [SMILES: C#Cc1cnc(Cl)c(N)c1-c1cc(Cl)c(C(=O)Nc2ccnc(C(F)(F)F)c2)cc1F] | 4-(3-amino-2-chloro-5- ethynylpyridin-4-yl)-2- chloro-5-fluoro-N-(2- (trifluoromethyl)pyridin-4- yl)benzamido | 469 |
| Example 568 (A568) | 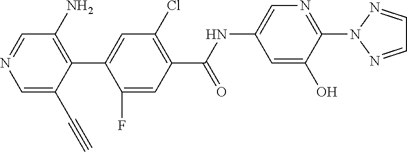 [SMILES: C#Cc1cncc(N)c1-c1cc(Cl)c(C(=O)Nc2cnc(-n3nccn3)c(O)c2)cc1F] | 4-(3-amino-5- ethynylpyridin-4-yl)-2- chloro-5-fluoro-N-(5- hydroxy-6-(2H-1,2,3- triazol-2-yl)pyridin-3- yl)benzamide | 450 |
| Example 569 (A569) | 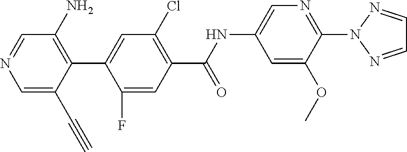 [SMILES: C#Cc1cncc(N)c1-c1cc(Cl)c(C(=O)Nc2cnc(-n3nccn3)c(OC)c2)cc1F] | 4-(3-amino-5- ethynylpyridin-4-yl)-2- chloro-5-fluoro-N-(5- methoxy-6-(2H-1,2,3- triazol-2-yl)pyridin-3- yl)benzamido | 464 |
| Example 570 (A570) | 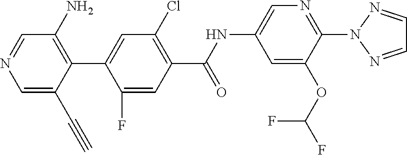 [SMILES: C#Cc1cncc(N)c1-c1cc(Cl)c(C(=O)Nc2cnc(-n3nccn3)c(OC(F)F)c2)cc1F] | 4-(3-amino-5- ethynylpyridin-4-yl)-2- chloro-N-(5- (difluoromethoxy)-6-(2H- 1,2,3-triazol-2-yl)pyridin-3- yl)-5-fluorobenzamide | 500 |
| Example 571 (A571) | 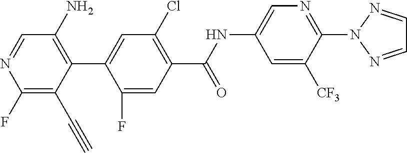 [SMILES: C#Cc1c(F)ncc(N)c1-c1cc(Cl)c(C(=O)Nc2cnc(-n3nccn3)c(C(F)(F)F)c2)cc1F] | N-(6-(2H-1,2,3-triazol-2-yl)- 5-(trifluoromethyl)pyridin-3- yl)-4-(5-amino-3-ethynyl-2- fluoropyridin-4-yl)-2-chloro- 5-fluorobenzamide | 520 |

### A410:

¹H NMR (500 MHz, DMSO-d₆) δ 11.65 (s, 1H), 9.17 (d, J=2.4 Hz, 1H), 8.93 (d, J=2.4 Hz, 1H), 8.25-8.15 (m, 4H), 7.91 (d, J=9.0 Hz, 1H), 7.71 (d, J=6.0 Hz, 1H), 5.76 (s, 2H), 4.50 (s, 1H).

### A545:

¹H NMR (500 MHz, DMSO-d₆) δ 11.57 (s, 1H), 9.15 (d, J=2.4 Hz, 1H), 8.91 (d, J=2.4 Hz, 1H), 8.21 (d, J=1.7 Hz, 2H), 7.95-7.83 (m, 2H), 7.69 (d, J=6.0 Hz, 1H), 5.73 (s, 2H).

### A571:

¹H NMR (500 MHz, DMSO-d₆) δ 11.60 (s, 1H), 9.14 (d, J=2.4 Hz, 1H), 8.91 (d, J=2.4 Hz, 1H), 8.21 (s, 2H), 7.88 (d, J=9.0 Hz, 1H), 7.75-7.66 (m, 2H), 5.29 (s, 2H), 4.50 (s, 1H).

### PHARMACOLOGICAL EXPERIMENTS

### Example A: MALT1

Test compounds of the present invention (in 100% DMSO) were spotted into a 384-well dilution plate and serially diluted to the required concentrations with DMSO. Then, 0.2 μL of the test compounds were transferred into a new 384-well reaction plate by Echo following centrifugation. The MALT enzyme and its substrate, AMC-labeled peptide, were prepared in 1× protease buffer to yield 2× enzyme solution and 2× peptide solution. Subsequently, 10 μL of 2× enzyme solution was added into the 384-well reaction plate, incubating with the test compounds for 15 minutes at 25° C. After that, 10 μL of 2× peptide solution was added into the plate, continuing to incubate for 90 minutes at 25° C. The plate was then placed on an Envision multifunctional microplate reader to collect raw data, which would be converted into inhibition rate:

Inhibition rate %=(max−sample)/(max−min)*100%.
“max” values were measured using the reaction with enzyme but no compound treated, while “min” values were generated from control wells containing assay buffer without enzyme.

Curve fitting was done with Graphpad Prism software to determine the IC₅₀ values. The specific experimental operations are as follows:

### 1. Protease Buffer Preparation (200 mL):

| | | Assay buffer |
|---|---|---|
| | | final |
| Stock solution | Volume | concentration |

| | | | | |
|---|---|---|---|---|
| 1M HEPES solution | 10 | mL | 50 | mM |
| 5M NaCl solution | 6 | mL | 150 | mM |

| | | | |
|---|---|---|---|
| 1% CHAPS solution | 10 | mL | 0.05% |
| Na Citrate•2H₂O powder | 58.82 | g | 1M |

| (MW = 294.10 g/mol) | | | |
|---|---|---|---|
| 1M DTT | Added | 1 | mM |

before use

58.82 g Na Citrate·2H₂O powder was added into 140 mL ddH₂O and mixed in a 37° C. water bath. Once there were no visible particles, the remaining solutions were added successively with continuous stirring for 10 minutes. The final volume was adjusted to 200 mL. The buffer was stored at room temperature. DTT was added before use.

### 2. Preparation of Reaction Plate:

1) All test compounds were diluted from 10 mM stock solution into 1 mM in a 384 well dilution plate (labcyte, PP-0200);
2) Ten various concentrations of the diluted compound solution above were obtained by serial dilution at an equal ratio of 1:3;
3) 0.2 μL DMSO was transferred to two empty wells of the 384 reaction plate as blank controls without compound and enzyme respectively;
4) 0.2 μL of the test compound at different concentrations was also transferred into the 384 reaction plate by Echo, with two duplicate wells for each concentration, then the plate was centrifuged at 1000 rpm for 1 min.

### 3. Enzymatic Reaction:

1) The MALT1 protease and AMC-labeled peptide was prepared in 1× protease buffer to obtain 2× enzyme solution and 2× peptide solution. 10 μL of 2× enzyme solution was added into the 384-well reaction plate. The plate was centrifuged at 1000 rpm for 1 min and incubated for 15 min at 25° C.
2) 10 μL of 2× peptide solution was then transferred to the 384 reaction plate, and the plate was centrifuged at 1000 rpm for 1 min and incubate for 90 min at 25° C. The final concentration of the compounds were 10.00, 3.33, 1.11, 0.37, 0.12, 0.04, 0.014, 0.0046, 0.0015, 0.0005, 0 μM.
3) The 360/460 signal value of the reaction plate was collected using the Envision multifunctional microplate reader to calculated the inhibition rate, which was fitted to determine the IC₅₀.

The results are expressed with IC₅₀, wherein “A” stands for “IC₅₀≤10 nM”, and “B” stands for “10 nM<IC₅₀≤100 nM”, and “C” stands for “100 nM<IC₅₀≤500 nM”, and “D” stands for “IC₅₀>500 nM”. The IC₅₀ data for MALT1 inhibition activity of representative compounds are shown in Table 1.

TABLE 1

| | Ac-LRSR-AMC |
|---|---|
| Compound | (IC₅₀, nM) |
| A109 | A |
| A110 | A |
| A113 | A |
| A114 | A |
| A115 | A |
| A116 | A |
| A119 | A |
| A120 | A |
| A121 | A |
| A122 | A |
| A123 | A |
| A124 | A |
| A126 | A |
| A127 | A |
| A281 | A |
| A324 | A |
| A335 | A |
| A338 | A |
| A346 | A |
| A342 | A |
| A333 | A |
| A409 | A |
| A410 | A |
| A411 | A |
| A412 | A |
| A413 | A |
| A414 | B |
| A415 | B |
| A416 | C |
| A417 | B |
| A418 | D |
| A419 | D |
| A420 | D |
| A421 | D |
| A422 | D |
| A423 | B |
| A424 | B |
| A425 | B |
| A426 | A |
| A427 | A |
| A428 | A |
| A429 | A |
| A430 | A |
| A431 | B |
| A432 | B |
| A433 | B |
| A434 | B |
| A435 | B |
| A436 | C |
| A437 | B |
| A438 | B |
| A439 | D |
| A440 | B |
| A441 | B |
| A442 | B |
| A443 | A |
| A444 | A |
| A445 | B |
| A446 | A |
| A447 | B |
| A448 | B |
| A449 | B |
| A450 | B |
| A451 | B |
| A452 | C |
| A453 | C |
| A454 | C |
| A455 | B |
| A456 | B |
| A457 | B |
| A458 | D |
| A459 | C |
| A460 | B |
| A461 | B |
| A462 | B |
| A463 | B |
| A464 | B |
| A465 | B |
| A466 | B |
| A467 | C |
| A468 | C |
| A469 | B |
| A470 | A |
| A471 | A |
| A472 | A |
| A475 | A |
| A478 | A |
| A479 | A |
| A485 | A |
| A486 | A |
| A504 | A |
| A505 | A |
| A506 | A |
| A507 | A |
| A517 | A |
| A518 | A |
| A521 | A |
| A551 | A |
| A552 | A |
| A553 | A |
| A554 | A |
| A556 | A |
| A557 | A |
| A558 | A |

According to Table 1, the compounds of the present invention exhibit strong inhibitory activities on MALT1.

### Example B: ELISA Method to Detect IL-2 Secretion Induced by PMA/Ionomycin

12.5*10³ Jurkat cells were seeded on a 96-well plate and incubated with various concentrations of compound solution for 30 min. Then, 500×PMA/Ionomycin stimulating factor was diluted with culture medium and added to the cell plate. The final concentrations of PMA/Ionomycin were 81 nM and 1.34 M respectively. The final concentrations of the compounds were 10000, 3333.3, 1111.1, 370.4, 123.5, 41.2, 13.7, 4.6, 1.5, 0 nM (The final concentration of DMSO is 0.5%). After incubation for 20 h, the supernatant was collected via centrifugation. The final IL-2 concentration was detected by ELISA method (IL-2 Duoset, R&D Systems, DY202). The signal value of each sample at a wavelength of 450 nm was collected by EnVision multifunctional microplate reader and converted to the IL-2 concentration via the ELISACalc.exe software. The IL-2 secretion inhibitory activity IC₅₀ was fitted by GraphPad Prism software. The cell viability was tested simultaneously using the Cell-Titer Glo kit.

The compounds of the present invention show strong inhibitory effect on IL-2 secretion of Jurkat cells (PMA/Ionomycin inducing). The IC₅₀ data of representative compounds for inhibiting IL-2 secretion of Jurkat cells are shown in Table 2.

TABLE 2

| | IL-2 secretion |
|---|---|
| Compound | (IC₅₀, nM) |
| A12 | 285 |
| A16 | 194 |
| A17 | 191 |
| A18 | 598 |
| A19 | 632 |
| A42 | 471 |
| A64 | 463 |
| A67 | 325 |
| A69 | 195 |
| A100 | 369 |
| A102 | 154 |
| A104 | 294 |
| A105 | 134 |
| A106 | 265 |
| A109 | 94 |
| A110 | 186 |
| A111 | 283 |
| A112 | 234 |
| A113 | 35 |
| A114 | 34 |
| A115 | 40 |
| A116 | 54 |
| A117 | 184 |
| A118 | 82 |
| A119 | 466 |
| A120 | 57 |
| A121 | 43 |
| A122 | 121 |
| A123 | 58 |
| A124 | 252 |
| A126 | 136 |
| A127 | 444 |
| A281 | 77 |
| A410 | 66 |
| A411 | 17 |
| A412 | 32 |
| A426 | 77 |
| A427 | 77 |
| A428 | 164 |
| A429 | 259 |
| A470 | 113 |
| A505 | 36 |
| A506 | 76 |
| A529 | 208 |

### Example C: Liver Microsome Metabolism Stability Assay

### 1. Test Buffer Preparation:

1900 mg MgCl₂ was dissolved into a final volume of 400 mL ultrapure water. 17.42 g K₂HPO₄ and 13.65 g KH₂PO₄ were dissolved respectively into ultrapure water with a final volume of 1000 mL.

The K₂HPO₄ and KH₂PO₄ stock solutions above were mixed with pH adjusted to 7.30±0.10 to obtain 100 mM potassium phosphate (K-PBS) buffer.

### 2. Reaction Stop Solution Preparation:

The stop solution was acetonitrile containing 1 ng/mL labetalol and 1 ng/mL glyburide, and stored at 4° C.

### 3. Working Solution Preparation:

The verapamil (positive control) and test sample stock solution were diluted to 50 μM and 200 μM respectively with MeOH/ACN/H₂O solution (1:1:2, volume ratio).

### 4. Experiment Procedure:

1) 40 μL MgCl₂ and 306 μL K-buffer were mixed in 96 plate wells containing blank control wells and test compound wells (the compound test wells without NADPH);
2) 4 μL compound working solution was added to each well (blank wells correspond to 4 μL K-buffer) (Note: The final concentration of DMSO volume in the system was ≤0.5%);
3) 10 μL liver microsomes (concentration: 20 mg/mL) was then added to each well, and the mixture was incubated at 37° C. for 10 min;
4) 40 μL NADPH working solution was added to each well to start the reaction, and the final total reaction volume reached 400 μL;
5) 50 μL samples from the reaction solution were aspirated out at 0, 5, 15, and 45 minutes, and added into 400 μL stop solution to terminate the reaction respectively;
6) The sample plate was placed in a shaker at 600 rpm for about 5 minutes at room temperature, then centrifuged at 4000 rpm for 10 minutes at 4° C.;
7) An aliquot of 50 μL of the supernatant after centrifugation was transferred to the pre-added 20% acetonitrile water, and shaked on a shaker at 600 rpm for 2 minutes at room temperature to mix well, and then analyzed using LC-MS/MS method.

### 5. Data Analysis:

The T_{1/2} and CL was calculated using the first-order kinetic equation:
the first-order kinetic equation:

Cₜ=C₀*e−Kt

Cₜ=(½)*C₀

T_{1/2}=ln 2/k=0.693/k

Slope k was determined from natural logarithmic linear regression of percent parent drug remaining versus incubation time.

The in vitro half-life (in vitro T_{1/2}) was calculated from the slope value:

$$\\ in vitro {T}_{1 / 2} = - ( 0.693 / k )$$

The in vitro intrinsic clearance rate (in vitro CLᵢₙₜ, in μL/min/mg proteins) was calculated according to the following formula (repeated average):

$$in vitro {CL}_{int} = \frac{0.693}{{T}_{1 / 2}} * \frac{volume of incubation ( μL )}{amount of proteins ( mg )}$$

Test positive control: verapamil.

Any compound value not within the specified range would be excluded and repeat experiment.

The compounds of the present invention had moderate or slow metabolism in three species: humans, rats, and mice, and the compounds of the present invention had good stability in liver microsomes in vitro of humans, rats.

### Example D: Mice Pharmacokinetic Test

The female BALB/c mice (20-30 g) was purchased from Beijing Weitonglihua Experimental Animal Technology Co., Ltd. to study the pharmacokinetic characteristics in mice (intravenous administration of 1 mg/kg and intragastric administration of 5 mg/kg).

### 1. Compound Solution Preparation:

| | | Dosing | Compound | |
|---|---|---|---|---|
| Route of | Dosage | volume | concentration | |
| administration | (mg/kg) | (mL/kg) | (mg/mL) | Solvent |
| IV | 1 | 5 | 0.2 | 5% DMSO/5% |
| tail vein | | | | Solutol/ |
| administration | | | | 90% saline |
| PO | 5 | 10 | 0.5 | 5% DMSO/5% |
| Oral | | | | Solutol/ |
| gavage | | | | 90% water |

Note:
All solutions need to be prepared one hour before administration.

### 2. Sample Collection and Storage:

The blood was collected at 5 min (intravenous administration only), 15 min, 30 min, 1 h, 2 h, 4 h, 7 h and 24 h after administration through the orbital venous plexus into a centrifuge tube containing EDTA anticoagulant, and 100 μL of whole blood was collected at each time point. After the centrifugation, the plasma was separated and stored in a −80° C. refrigerator until analysis.

### 3. Sample Processing and Testing:

The above samples were processed by the protein precipitation method. The standard curve and other samples were also prepared in the same way. The precipitated samples were centrifuged at 3200 rpm for 10 minutes at 4° C. The supernatant was aspirated and mixed with water 1:1, and then analyzed by LC-MS/MS. The quantitative limit of the standard curve of this compound in BALB/c mouse plasma was 1.00-1000 ng/mL.

### 4. Data Analysis:

The non-compartmental model of Phoenix WinNonlin software was used to calculate the pharmacokinetic parameters of animals after drug administration.

The compound of the present invention had higher exposure and higher bioavailability after oral administration in female BALB/c mice.

1. A compound of Formula I, or a stereoisomer, tautomer, deuterated compound, pharmaceutically acceptable salt, prodrug, chelate, non-covalent complex or solvate thereof, 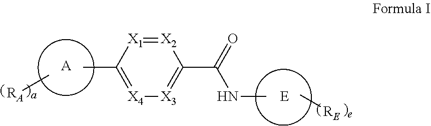
wherein,
X₁ is selected from CR₁ or N;
X₂ is selected from CR₂ or N;
X₃ is selected from CR₃ or N;
X₄ is selected from CR₄ or N;
R₁, R₂, R₃, R₄ are the same or different, and are each independently selected from the group consisting of H, CN, NO₂, halogen, —C₁₋₆ alkyl, —C₂₋₆ alkenyl, —C₂₋₆ alkynyl, —OR₅, —NR₅R₆, SR₅, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, —C(O)R₅, —N(R₅)C(O)R₆, —C(O)NR₅R₆, —N(R₅)C(O)OR₆, —OC(O)NR₅R₆, —N(R₅)C(O)NR₆R₇, —S(O)R₅, —S(O)₂R₅, —S(O)NR₅R₆, —S(O)₂NR₅R₆, —N(R₅)S(O)R₆, —N(R₅)S(O)₂R₆, —C(O)OR₅, —OC(O)R₅ and —OC(O)OR₅; wherein the —C₁₋₆ alkyl, —C₂₋₆ alkenyl, —C₂₋₆ alkynyl, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, R₅, R₆ and R₇ are optionally substituted with one or more substituents selected from hydrogen, halogen, CN, —OR₅, —NR₈R₉, —SR₈, —C(O)R₈, —N(R₈)C(O)R₉, —C(O)NR₈R₉, —N(R₈)C(O)OR₉, —OC(O)NR₈R₉, —N(R₈)C(O)NR₉R₁₀, —S(O)R₈, —S(O)₂R₈, —S(O)NR₈R₉, —S(O)₂NR₈R₉, —N(R₈)S(O)R₉, —N(R₈)S(O)₂R₉, —C(O)OR₈, —OC(O)R₈, —OC(O)OR₈, oxo, —C₁₋₆ alkyl, —C₂₋₆ alkenyl, —C₂₋₆ alkynyl, —C₀₋₆ alkyl-C₃₋₈ carbocyclyl or —C₀₋₆ alkyl-3-8 membered heterocyclyl;
ring A is selected from —C₅₋₆ carbocyclyl, 5-6 membered heterocyclyl, —C₆ aryl or 5-6 membered heteroaryl;
each R_{A} is the same or different, and is independently selected from the group consisting of hydrogen, halogen, oxo, CN, NO₂, —C₁₋₆ alkyl, —C₂₋₆ alkenyl, —C₂₋₆ alkynyl, —OR₅, —NR₅R₆, —SR₅, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, —C(O)R₅, —N(R₅)C(O)R₆, —C(O)NR₅R₆, —N(R₅)C(O)OR₆, —OC(O)NR₅R₆, —N(R₅)C(O)NR₆R₇, —S(O)R₅, —S(O)₂R₅, —S(O)NR₅R₆, —S(O)₂NR₅R₆, —N(R₅)S(O)R₆, —N(R₅)S(O)₂R₆, —C(O)OR₅, —OC(O)R₅ and —OC(O)OR₅; wherein the —C₁₋₆ alkyl, —C₂₋₆ alkenyl, —C₂₋₆ alkynyl, —C₀₋₆ alkyl-C₃₋₈ carbocyclyl, —C₀₋₆ alkyl-3-8 membered heterocyclyl, —C₀₋₆ alkyl-C₆₋₁₀ aryl, —C₀₋₆ alkyl-5-10 membered heteroaryl, R₅, R₆ and R₇ are optionally substituted with one or more substituents selected from hydrogen, halogen, CN, oxo, —C₁₋₆ alkyl, —C₂₋₆ alkenyl, —C₂₋₆ alkynyl, —C₀₋₆ alkyl-C₃₋₈ carbocyclyl, —C₀₋₆ alkyl-3-8 membered heterocyclyl, —OR₈, —NR₈R₉, —SR₈, —S(O)R₈, —S(O)₂R₈, —C(O)R₈, —C(O)OR₈, —OC(O)R₈, —N(R₈)C(O)R₉ or —C(O)NR₈R₉;
ring E is selected from 5-10 membered heterocyclyl, C₆₋₁₀ aryl or 5-10 membered heteroaryl;
each R_{E} is the same or different, and is independently selected from the group consisting of hydrogen, halogen, oxo, CN, NO₂, —C₁₋₆ alkyl, —C₂₋₆ alkenyl, —C₂₋₆ alkynyl, —C₀₋₆ alkyl-5-10 membered heteroaryl, —C₀₋₆ alkyl-C₃₋₈ carbocyclyl, —C₀₋₆ alkyl-3-8 membered heterocyclyl, —OR₅, —NR₅R₆, —SR₅, —C(O)R₅, —N(R₅)C(O)R₆, —C(O)NR₅R₆, —N(R₅)C(O)OR₆, —OC(O)NR₅R₆, —N(R₅)C(O)NR₆R₇, —S(O)R₅, —S(O)₂R₅, —S(O)NR₅R₆, —S(O)₂NR₅R₆, —N(R₅)S(O)R₆, —N(R₅)S(O)₂R₆, —C(O)OR₅, —OC(O)R₆ and —OC(O)OR₇; wherein the —C₁₋₆ alkyl, —C₂₋₆ alkenyl, —C₂₋₆ alkynyl, —C₀₋₆ alkyl-5-10 membered heteroaryl, —C₀₋₆ alkyl-C₃₋₈ carbocyclyl, —C₀₋₆ alkyl-3-8 membered heterocyclyl, R₅, R₆ and R₇ are optionally substituted with one or more substituents selected from hydrogen, halogen, CN, oxo, —C₁₋₆ alkyl, —C₂₋₆ alkenyl, —C₂₋₆ alkynyl, —OR₅, —NR₈R₉, substituted or unsubstituted —C₀₋₆ alkyl-C₃₋₆ carbocyclyl, substituted or unsubstituted —C₀₋₆ alkyl-3-6 membered heterocyclyl, —C(O)OR₅ or —C(O)NR₈R₉;
R₅, R₆, R₇, R₈, R₉, R₁₀ are each independently selected from the group consisting of hydrogen, halogen, CN, OH, NH₂, oxo, —C₁₋₆ alkyl, —C₁₋₆ alkoxy, —C₂₋₆ alkenyl, —C₂₋₆ alkynyl, —C₀₋₆ alkyl-C₃₋₁₄ carbocyclyl, —C₀₋₆ alkyl-3-14 membered heterocyclyl; wherein the —C₁₋₆ alkyl, —C₁₋₆ alkoxy, —C₂₋₆ alkenyl, —C₂₋₆ alkynyl, —C₀₋₆ alkyl-C₃₋₁₄ carbocyclyl, —C₀₋₆ alkyl-3-14 membered heterocyclyl are optionally substituted with one or more substituents selected from hydrogen, halogen, CN, OH, NH₂, —COOH, oxo, —C₁₋₆ alkyl, —C₁₋₆ haloalkyl, —C₂₋₆ alkenyl, —C₂₋₆ alkynyl, —C₁₋₆ alkoxy, —C₁₋₆ haloalkoxy, —NH(C₁₋₆ alkyl), —N(C₁₋₆ alkyl)₂, —COO(C₁₋₆ alkyl), —CONH(C₁₋₆ alkyl), —CON(C₁₋₆ alkyl)₂, substituted or unsubstituted —C₀₋₆ alkyl-C₃₋₆ carbocyclyl, substituted or unsubstituted —C₀₋₆ alkyl-3-6 membered heterocyclyl;
a is selected from 0, 1, 2, 3, 4, 5 or 6;
e is selected from 0, 1, 2, 3, 4, 5 or 6;
when X₁ is selected from CH, X₂ is selected from CH, X₃ is selected from CH, and X₄ is selected from CH, 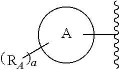
is not phenyl.

2. The compound of claim 1, or a stereoisomer, tautomer, deuterated compound, pharmaceutically acceptable salt, prodrug, chelate, non-covalent complex, or solvate thereof, wherein 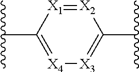
is selected from

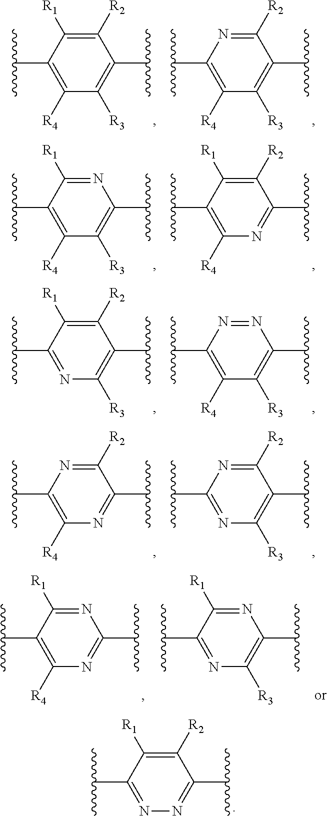

3. The compound of claim 1, or a stereoisomer, tautomer, deuterated compound, pharmaceutically acceptable salt, prodrug, chelate, non-covalent complex or solvate thereof, wherein 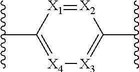
is selected from

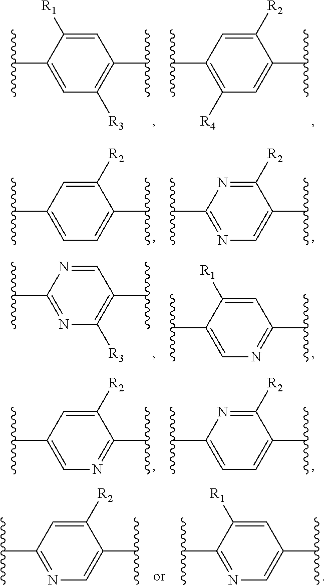

4. The compound of claim 1, or a stereoisomer, tautomer, deuterated compound, pharmaceutically acceptable salt, prodrug, chelate, non-covalent complex or solvate thereof, wherein R₁, R₃ are the same or different, and are each independently selected from the group consisting of halogen, —C₁₋₆ alkyl, —OR₅, —NR₅R₆, —C₀₋₆ alkyl-C₃₋₈ carbocyclyl and —C₀₋₆ alkyl-3-8 membered heterocyclyl; wherein the —C₁₋₆ alkyl, —C₀₋₆ alkyl-C₃₋₈ carbocyclyl, —C₀₋₆ alkyl-3-8 membered heterocyclyl, R₅ and R₆ are optionally substituted with one or more substituents selected from hydrogen, halogen, CN, OH, NH₂, —C₁₋₆ alkyl, —C₂₋₆ alkenyl, —C₂₋₆ alkynyl, —C₁₋₆ alkoxy, —C₀₋₆ alkyl-C₃₋₈ carbocyclyl or —C₀₋₆ alkyl-3-8 membered heterocyclyl.

5. The compound of claim 1, or a stereoisomer, tautomer, deuterated compound, pharmaceutically acceptable salt, prodrug, chelate, non-covalent complex or solvate thereof, wherein R₂, R₄ are the same or different, and are each independently selected from the group consisting of halogen, —C₁₋₆ alkyl, —OR₅, —NR₅R₆, —C₀₋₆ alkyl-C₃₋₈ carbocyclyl and —C₀₋₆ alkyl-3-8 membered heterocyclyl; wherein the —C₁₋₆ alkyl, —C₀₋₆ alkyl-C₃₋₈ carbocyclyl, —C₀₋₆ alkyl-3-8 membered heterocyclyl, R₅ and R₆ are optionally substituted with one or more substituents selected from hydrogen, halogen, CN, OH, NH₂, —C₁₋₆ alkyl, —C₂₋₆ alkenyl, —C₂₋₆ alkynyl, —C₁₋₆ alkoxy, —C₀₋₆ alkyl-C₃₋₈ carbocyclyl or —C₀₋₆ alkyl-3-8 membered heterocyclyl.

6. The compound of claim 1, or a stereoisomer, tautomer, deuterated compound, pharmaceutically acceptable salt, prodrug, chelate, non-covalent complex or a solvate thereof, wherein R₁, R₂, R₃, R₄ are the same or different, and are each independently selected from the group consisting of H, —F, —Cl, —Br, —I, —OH, —NH₂, —CN, —CH₃, —CF₃, —CH₂CH₃, —CH(CH₃)₂, —CH₂CH(CH₃)₂, —C(CH₃)₃, —NHCH(CH₃)₂, —NHC(O)CH₃, —NHC(O)OCH₂CH₃, 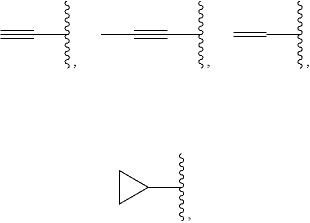
—OCH₃, —OCH(CH₃)₂,

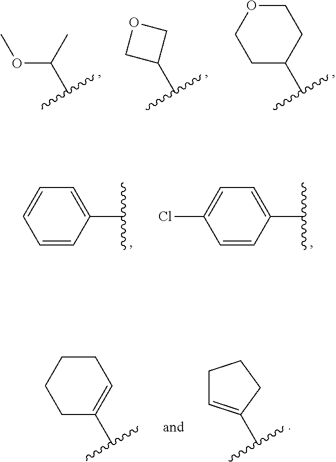

7. The compound of claim 1, or a stereoisomer, tautomer, deuterated compound, pharmaceutically acceptable salt, prodrug, chelate, non-covalent complex or solvate thereof, wherein ring A is selected from —C₆ carbocyclyl, 6 membered heterocyclyl, —C₆ aryl or 6 membered heteroaryl; preferably, ring A is 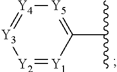
wherein Y₁ is selected from CR_{A} or N; Y₂ is selected from CR_{A} or N; Y₃ is selected from CR_{A} or N; Y₄ is selected from CR_{A} or N; R_{A} is defined as described in any one of claims 1-6.

8. The compound of claim 1, or a stereoisomer, tautomer, deuterated compound, pharmaceutically acceptable salt, prodrug, a chelate, non-covalent complex or solvate thereof, wherein ring A is selected from phenyl, pyridyl, furyl, thienyl, pyrrolyl, pyrazolyl, pyrimidinyl, pyridazinyl, diazinyl, cyclopentenyl, pyridin-2(1H)-one or pyrimidine-2(1H)-keto group.

9. The compound of claim 1, or a stereoisomer, tautomer, deuterated compound, pharmaceutically acceptable salt, prodrug, chelate, non-covalent complex or a solvate thereof, wherein each R_{A} is the same or different, and is independently selected from the group consisting of hydrogen, oxo, —F, —Cl, —Br, —I, —CN, —OH, —NH₂, —NO₂, —CH₃, —CF₃, —OCF₃, —NHCH₃, —N(CH₃)₂, —OCH₃, —SCH₃, —CHF₂, —OCHF₂, —C(O)NH₂, —C(O)CH₃, —OC(O)NH₂, —NHC(O)CH₃, —CH₂CH₃, —CH(CH₃)₂, 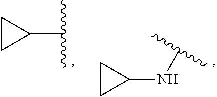
—SCH₃, —SOCH₃, —CH₂SO₂CH₃, —SO₂CH₃, —NHC(O)NH₂, —CH₂OH, —CH₂NH₂, —NHC(O)OCH₂CH₃, —NHOH, —CH₂OCH₃,

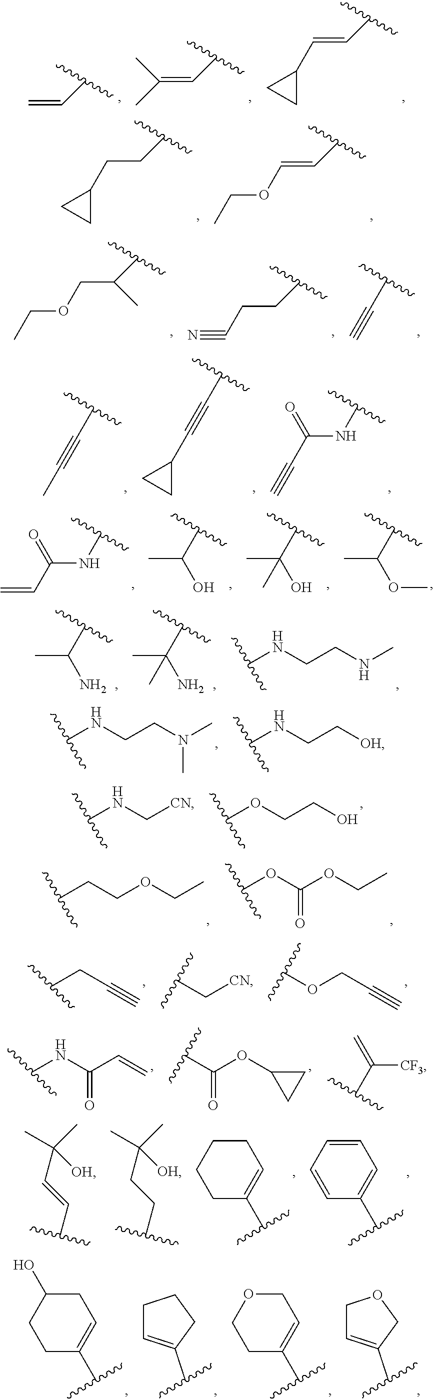

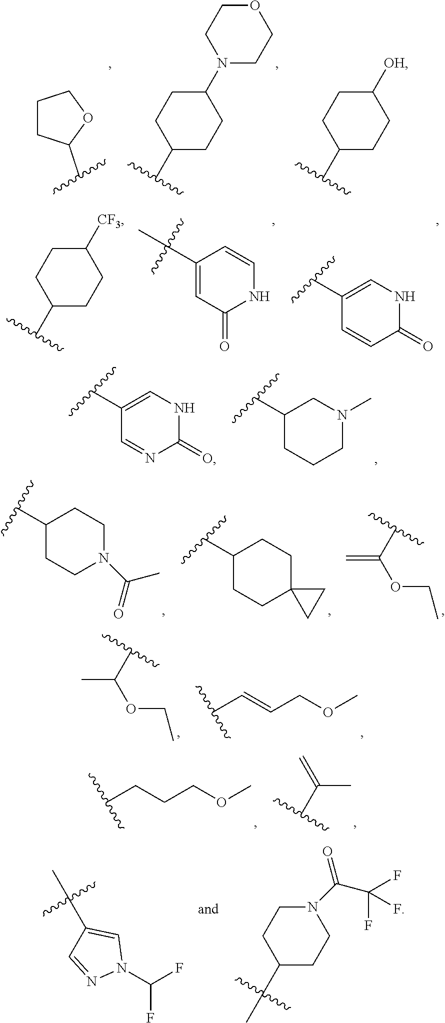

10. The compound of claim 1, or a stereoisomer, tautomer, deuterated compound, pharmaceutically acceptable salt, prodrug, chelate, non-covalent complex or a solvate thereof, wherein ring E is selected from 6-10 membered heterocyclyl or 6-10 membered heteroaryl.

11. The compound of claim 1, or stereoisomer, tautomer, deuterated compound, pharmaceutically acceptable salt, prodrug, chelate, non-covalent complex or solvate thereof, wherein ring E is selected from pyridyl, benzo[d]oxazolyl, 3,4-dihydro-2H-benzo[b][1,4]oxazinyl, 1,6-naphthyridinyl, 2,3-dihydro-1H-pyrido[2,3-b][1,4]oxazinyl, 1H-pyrazolo[4,3-b]pyridyl, 2H-pyrazolo[4,3-b]pyridyl, 1H-pyrazolo[3,4-b]pyridyl, pyrazolo[1,5-a]pyrimidinyl, imidazo[1,2-b]pyridazinyl, thiazolo[5,4-b] pyridyl or 1,5-naphthyridinyl.

12. The compound of claim 1, or a stereoisomer, tautomer, deuterated compound, pharmaceutically acceptable salt, prodrug, chelate, non-covalent complex or solvate thereof, wherein each R_{E} is the same or different, and is independently selected from the group consisting of hydrogen, halogen, oxo, CN, —C₁₋₆ alkyl, —C₀₋₆ alkyl-5-10 membered heteroaryl, —C₀₋₆ alkyl-C₃₋₈ carbocyclyl, —C₀₋₆ alkyl-3-8 membered heterocyclyl, —OR₅, —NR₅R₆, —SR₅, —C(O)R₅, —S(O)R₅, —S(O)₂R₅ and —C(O)OR₅; wherein the —C₁₋₆ alkyl, —C₀₋₆ alkyl-5-10 membered heteroaryl, —C₀₋₆ alkyl-C₃₋₈ carbocyclyl and —C₀₋₆ alkyl-3-8 membered heterocyclyl are optionally substituted with one or more substituents selected from hydrogen, halogen, CN, oxo, —C₁₋₆ alkyl, —C₂₋₆ alkenyl, —C₂₋₆ alkynyl, —OR₅, —NR₈R₉, —C₀₋₆ alkyl —C₃₋₈ carbocyclyl and —C₀₋₆ alkyl-3-8 membered heterocyclyl, —C(O)OR₅ or —C(O)NR₈R₉.

13. The compound of claim 1, or a stereoisomer, tautomer, deuterated compound, pharmaceutically acceptable salt, prodrug, chelate, non-covalent complex or solvate thereof, wherein each R_{E} is the same or different, and is independently selected from the group consisting of hydrogen, oxo, —F, —Cl, —CN, —CH₃, —OCH₃, —CH₂OH, —CH₂CN, —CF₃, —C(O)NH₂, —C(O)NHCH₃, —C(O)NHCH₂CN, —C(O)NHCH₂CH₃, —C(O)NHCH₂CF₃, —C(O)NHCH₂CH₂OCF₃, —C(O)NHCH₂CH₂CH₃, —OCHF₂, —OCF₃, —SO₂CH₃, —SCH₃, —SCF₃, —NHC(O)NH₂, —NHC(O)CH₃, —NHC(O)OCH₃, —NHS(O)₂CH₃,

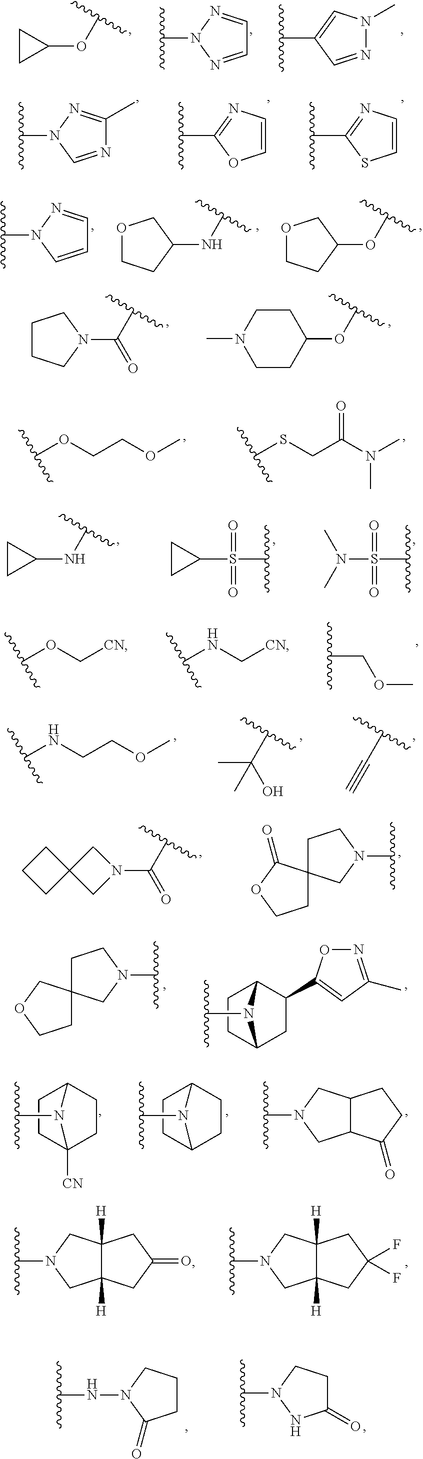

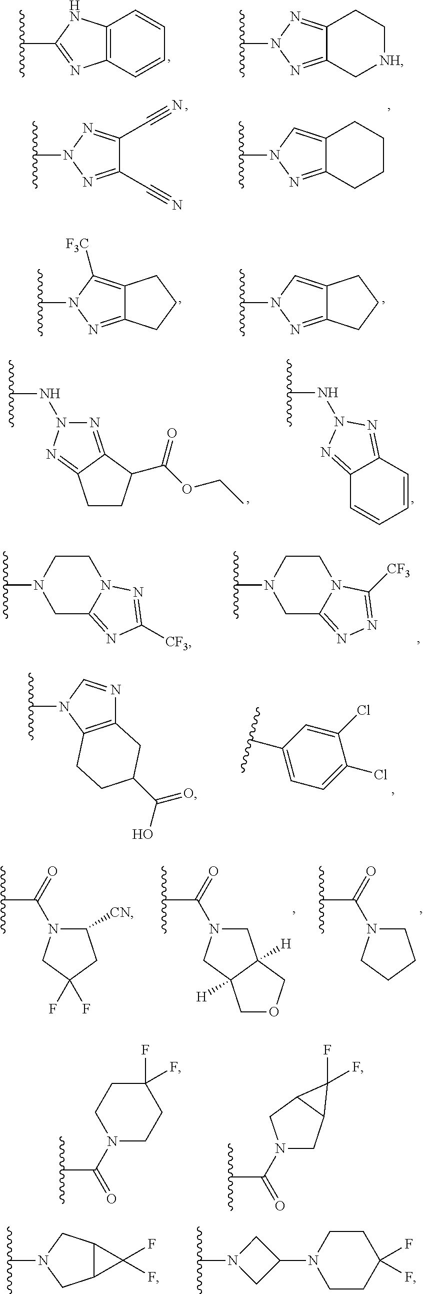

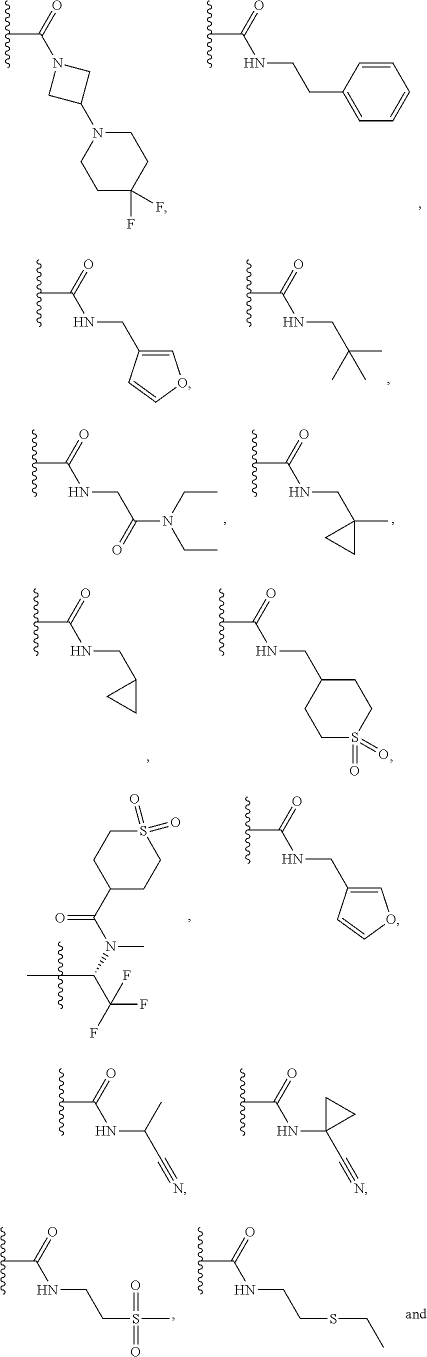

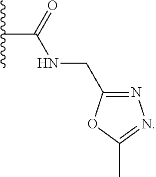 [SMILES: *C(=O)NCc1nnc(C)o1]

14. The compound of claim 1, or a stereoisomer, tautomer, deuterated compound, pharmaceutically acceptable salt, prodrug, chelate, non-covalent complex or solvate thereof, wherein 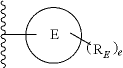
is selected from 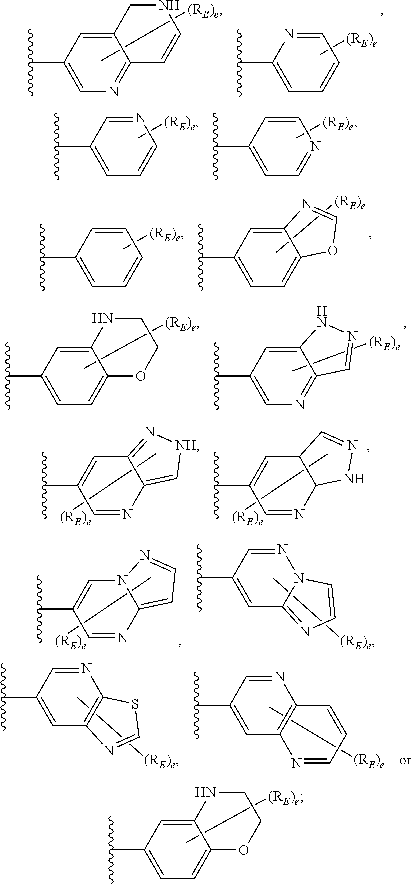
preferably, 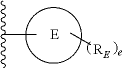
is selected from 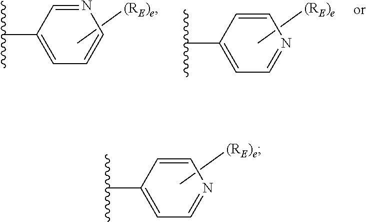
more preferably, 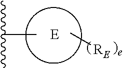
is selected from

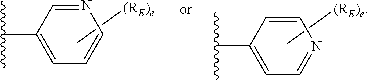

15. The compound of claim 1, or a stereoisomer, tautomer, deuterated compound, pharmaceutically acceptable salt, prodrug, chelate, non-covalent complex or solvate thereof, wherein the compound is selected from Formula II: 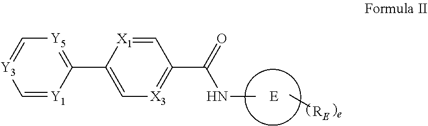
wherein, ring E, X₁, X₃, Y₁, Y₃, Y₅, R_{E}, e are defined as described in any one of claims 1-14.

16. The compound of claim 1, or a stereoisomer, tautomer, deuterated compound, pharmaceutically acceptable salt, prodrug, chelate, non-covalent complex or solvate thereof, wherein the compound is selected from Formula III: 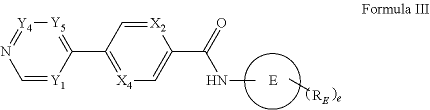
wherein, ring E, X₂, X₄, Y₁, Y₄, Y₅, R_{E}, e are defined as described in any one of claims 1-14.

17. The compound of claim 1, or a stereoisomer, a tautomer, a deuterated compound, a pharmaceutically acceptable salt, a prodrug, a chelate, a non-covalent complex, or a solvate thereof, wherein the compound is selected from:
N-(6-(2H-1,2,3-triazol-2-yl)-5-(trifluoromethyl)pyridin-3-yl)-5-(2-chloro-4-fluorophenyl)-4-methylpicolinamide;
N-(6-(2H-1,2,3-triazol-2-yl)-5-(trifluoromethyl)pyridin-3-yl)-2-fluoro-4-(3-methylpyridin-2-yl)benzamide;
N-(6-(2H-1,2,3-triazol-2-yl)-5-(trifluoromethyl)pyridin-3-yl)-2′-chloro-4′-fluoro-3-isopropyl-[1,1′-biphenyl]-4-carboxamide;
N-(6-(2H-1,2,3-triazol-2-yl)-5-(trifluoromethyl)pyridin-3-yl)-3,5′-dimethyl-[2,3′-bipyridine]-6′-carboxamide;
N-(6-(2H-1,2,3-triazol-2-yl)-5-(trifluoromethyl)pyridin-3-yl)-2-methoxy-4-(3-methylpyridin-2-yl)benzamide;
N-(6-(2H-1,2,3-triazol-2-yl)-5-(trifluoromethyl)pyridin-3-yl)-2′-chloro-4′-fluoro-3-methoxy-[1,1′-biphenyl]-4-carboxamide;
N-(6-(2H-1,2,3-triazol-2-yl)-5-(trifluoromethyl)pyridin-3-yl)-6-(2-chloro-4-fluorophenyl)-2-methoxynicotinamide;
N-(6-(2H-1,2,3-triazol-2-yl)-5-(trifluoromethyl)pyridin-3-yl)-2-(2-chloro-4-fluorophenyl)-4-methylpyrimidine-5-carboxamide;
N-(6-(2H-1,2,3-triazol-2-yl)-5-(trifluoromethyl)pyridin-3-yl)-4-methyl-2-(4-methylpyridin-3-yl)pyrimidine-5-carboxamide;
N-(6-(2H-1,2,3-triazol-2-yl)-5-(trifluoromethyl)pyridin-3-yl)-2′-chloro-4′-fluoro-3-methyl-[1,1′-biphenyl]-4-carboxamide;
N-(6-(2H-1,2,3-triazol-2-yl)-5-(trifluoromethyl)pyridin-3-yl)-2′,3-dichloro-4′-fluoro-[1,1′-biphenyl]-4-carboxamide;
N-(6-(2H-1,2,3-triazol-2-yl)-5-(trifluoromethyl)pyridin-3-yl)-4-chloro-6-(2-chloro-4-fluorophenyl)nicotinamide;
N-(6-(2H-1,2,3-triazol-2-yl)-5-(trifluoromethyl)pyridin-3-yl)-2′-chloro-3-cyclopropyl-4′-fluoro-[1,1′-biphenyl]-4-carboxamide;
N-(6-(2H-1,2,3-triazol-2-yl)-5-(trifluoromethyl)pyridin-3-yl)-2-(4-cyano-2-methylphenyl)-4-methylpyrimidine-5-carboxamide;
N-(6-(2H-1,2,3-triazol-2-yl)-5-(trifluoromethyl)pyridin-3-yl)-5-(2-chloro-4-fluorophenyl)-3-methylpicolinamide;
N-(6-(2H-1,2,3-triazol-2-yl)-5-(trifluoromethyl)pyridin-3-yl)-2-(2-chloro-4-fluorophenyl)-4-(trifluoromethyl)pyrimidine-5-carboxamide;
N-(6-(2H-1,2,3-triazol-2-yl)-5-(trifluoromethyl)pyridin-3-yl)-2′-chloro-3,4′-difluoro-[1,1′-biphenyl]-4-carboxamide;
N-(6-(2H-1,2,3-triazol-2-yl)-5-(trifluoromethyl)pyridin-3-yl)-4-methyl-2-(2-(trifluoromethyl)phenyl)pyrimidine-5-carboxamide;
N-(6-(2H-1,2,3-triazol-2-yl)-5-(trifluoromethyl)pyridin-3-yl)-6-(2-chloro-4-fluorophenyl)-5-methylnicotinamide;
2-(2-chloro-4-fluorophenyl)-N-(5-chloro-6-(2H-1,2,3-triazol-2-yl)pyridin-3-yl)-4-methylpyrimidine-5-carboxamide;
N-(6-(2H-1,2,3-triazol-2-yl)-5-(trifluoromethyl)pyridin-3-yl)-6-(5-amino-2-chlorophenyl)-2-methoxynicotinamide;
N-(6-(2H-1,2,3-triazol-2-yl)-5-(trifluoromethyl)pyridin-3-yl)-6-methoxy-3′-methyl-[2,2′-bipyridine]-5-carboxamide;
N-(6-(2H-1,2,3-triazol-2-yl)-5-(trifluoromethyl)pyridin-3-yl)-6-(2-chloro-4-fluorophenyl)-4-methoxynicotinamide;
3-chloro-N-(5-chloro-6-(2H-1,2,3-triazol-2-yl)pyridin-3-yl)-2′-fluoro-6′-methyl-[1,1′-biphenyl]-4-carboxamide;
3,3′-dichloro-N-(5-chloro-6-(2H-1,2,3-triazol-2-yl)pyridin-3-yl)-2′-methyl-[1,1′-biphenyl]-4-carboxamide;
5-(3-chloro-2-methylphenyl)-N-(5-chloro-6-(2H-1,2,3-triazol-2-yl)pyridin-3-yl)-3-methylpicolinamide;
5-(2-chloro-4-fluorophenyl)-N-(5-chloro-6-(2H-1,2,3-triazol-2-yl)pyridin-3-yl)-3-methylpicolinamide;
N-(6-(2H-1,2,3-triazol-2-yl)-5-(trifluoromethyl)pyridin-3-yl)-2′-chloro-3-methyl-[1,1′-biphenyl]-4-carboxamide;
N-(6-(2H-1,2,3-triazol-2-yl)-5-(trifluoromethyl)pyridin-3-yl)-2′-amino-3-methyl-[1,1′-biphenyl]-4-carboxamide;
N-(6-(2H-1,2,3-triazol-2-yl)-5-(trifluoromethyl)pyridin-3-yl)-4′-chloro-3-methyl-[1,1′-biphenyl]-4-carboxamide;
N-(6-(2H-1,2,3-triazol-2-yl)-5-(trifluoromethyl)pyridin-3-yl)-2′-chloro-3,3′-dimethyl-[1,1′-biphenyl]-4-carboxamide;
N-(6-(2H-1,2,3-triazol-2-yl)-5-(trifluoromethyl)pyridin-3-yl)-2′,3′-dichloro-3-methyl-[1,1′-biphenyl]-4-carboxamide;
N-(6-(2H-1,2,3-triazol-2-yl)-5-(trifluoromethyl)pyridin-3-yl)-2′-fluoro-3,6′-dimethyl-[1,1′-biphenyl]-4-carboxamide;
N4′-(6-(2H-1,2,3-triazol-2-yl)-5-(trifluoromethyl)pyridin-3-yl)-3′-methyl-[1,1′-biphenyl]-2,4′-dicarboxamide;
2-chloro-N-(5-chloro-6-(2H-1,2,3-triazol-2-yl)pyridin-3-yl)-4-(3-methylpyridin-4-yl)benzamide;
2′,3-dichloro-N-(5-chloro-6-(2H-1,2,3-triazol-2-yl)pyridin-3-yl)-[1,1′-biphenyl]-4-carboxamide;
3-chloro-N-(5-chloro-6-(2H-1,2,3-triazol-2-yl)pyridin-3-yl)-2′-(trifluoromethoxy)-[1,1′-biphenyl]-4-carboxamide;
3-chloro-N-(5-chloro-6-(2H-1,2,3-triazol-2-yl)pyridin-3-yl)-3′-cyano-[1,1′-biphenyl]-4-carboxamide;
N-(5-chloro-6-(2H-1,2,3-triazol-2-yl)pyridin-3-yl)-5-(3-chlorophenyl)-3-methylpicolinamide;
N-(6-(2H-1,2,3-triazol-2-yl)-5-(trifluoromethyl)pyridin-3-yl)-3′-chloro-3-methyl-[1,1′-biphenyl]-4-carboxamide;
N-(6-(2H-1,2,3-triazol-2-yl)-5-(trifluoromethyl)pyridin-3-yl)-2′-cyano-3-methyl-[1,1′-biphenyl]-4-carboxamide;
N-(6-(2H-1,2,3-triazol-2-yl)-5-(trifluoromethyl)pyridin-3-yl)-2′-fluoro-3-methyl-4′-(trifluoromethyl)-[1,1′-biphenyl]-4-carboxamide;
N-(6-(2H-1,2,3-triazol-2-yl)-5-(trifluoromethyl)pyridin-3-yl)-2-methyl-4-(6-(trifluoromethyl)pyridin-3-yl)benzamide;
N-(6-(2H-1,2,3-triazol-2-yl)-5-(trifluoromethyl)pyridin-3-yl)-4-(furan-2-yl)-2-methylbenzamide;
N-(6-(2H-1,2,3-triazol-2-yl)-5-(trifluoromethyl)pyridin-3-yl)-4-(cyclopent-1-en-1-yl)-2-methylbenzamide;
N-(6-(2H-1,2,3-triazol-2-yl)-5-(trifluoromethyl)pyridin-3-yl)-5-(2-cyanophenyl)-3-methylpicolinamide;
N-(6-(2H-1,2,3-triazol-2-yl)-5-(trifluoromethyl)pyridin-3-yl)-2-methyl-4-(1-methyl-1H-pyrazol-5-yl)benzamide;
N-(6-(2H-1,2,3-triazol-2-yl)-5-(trifluoromethyl)pyridin-3-yl)-2′-chloro-3-cyano-[1,1′-biphenyl]-4-carboxamide;
N-(6-(2H-1,2,3-triazol-2-yl)-5-(trifluoromethyl)pyridin-3-yl)-4′-chloro-3′-cyano-3-methyl-[1,1′-biphenyl]-4-carboxamide;
N-(6-(2H-1,2,3-triazol-2-yl)-5-(trifluoromethyl)pyridin-3-yl)-2-methyl-4-(4-methylpyridin-3-yl)benzamide;
N-(6-(2H-1,2,3-triazol-2-yl)-5-(trifluoromethyl)pyridin-3-yl)-2-methyl-4-(1-oxoisoindolin-5-yl)benzamide;
N-(6-(2H-1,2,3-triazol-2-yl)-5-(trifluoromethyl)pyridin-3-yl)-2-methyl-4-(quinolin-4-yl)benzamide;
N4′-(6-(2H-1,2,3-triazol-2-yl)-5-(trifluoromethyl)pyridin-3-yl)-3′-methyl-[1,1′-biphenyl]-3,4′-dicarboxamide;
N-(6-(2H-1,2,3-triazol-2-yl)-5-(trifluoromethyl)pyridin-3-yl)-2′-methoxy-3-(trifluoromethyl)-[1,1′-biphenyl]-4-carboxamide;
N-(6-(2H-1,2,3-triazol-2-yl)-5-(trifluoromethyl)pyridin-3-yl)-2-methyl-4-(1H-pyrrolo[2,3-b]pyridin-5-yl)benzamide;
N-(6-(2H-1,2,3-triazol-2-yl)-5-(trifluoromethyl)pyridin-3-yl)-2′-methoxy-3-methyl-[1,1′-biphenyl]-4-carboxamide;
N-(6-(2H-1,2,3-triazol-2-yl)-5-(trifluoromethyl)pyridin-3-yl)-4-(6-fluoropyridin-3-yl)-2-methylbenzamide;
N-(6-(2H-1,2,3-triazol-2-yl)-5-(trifluoromethyl)pyridin-3-yl)-4-(6-cyanopyridin-3-yl)-2-methylbenzamide;
N-(6-(2H-1,2,3-triazol-2-yl)-5-(trifluoromethyl)pyridin-3-yl)-5-chloro-2-fluoro-2′-methoxy-[1,1′-biphenyl]-4-carboxamide;
N-(6-(2H-1,2,3-triazol-2-yl)-5-(trifluoromethyl)pyridin-3-yl)-2′-amino-3-(trifluoromethyl)-[1,1′-biphenyl]-4-carboxamide;
N-(6-(2H-1,2,3-triazol-2-yl)-5-(trifluoromethyl)pyridin-3-yl)-2′-amino-5-chloro-2-fluoro-[1,1′-biphenyl]-4-carboxamide;
N-(6-(2H-1,2,3-triazol-2-yl)-5-(trifluoromethyl)pyridin-3-yl)-2′-acetamido-3-(trifluoromethyl)-[1,1′-biphenyl]-4-carboxamide;
N-(6-(2H-1,2,3-triazol-2-yl)-5-(trifluoromethyl)pyridin-3-yl)-4-(4-chloropyridin-3-yl)-2-(trifluoromethyl)benzamide;
N-(6-(2H-1,2,3-triazol-2-yl)-5-(trifluoromethyl)pyridin-3-yl)-2′-(dimethylamino)-3-(trifluoromethyl)-[1,1′-biphenyl]-4-carboxamide;
N-(6-(2H-1,2,3-triazol-2-yl)-5-(trifluoromethyl)pyridin-3-yl)-2′-(dimethylamino)-3-methyl-[1,1′-biphenyl]-4-carboxamide;
N-(6-(2H-1,2,3-triazol-2-yl)-5-(trifluoromethyl)pyridin-3-yl)-3-chloro-2′-(dimethylamino)-[1,1′-biphenyl]-4-carboxamide;
N-(6-(2H-1,2,3-triazol-2-yl)-5-(trifluoromethyl)pyridin-3-yl)-3-cyano-2′-(dimethylamino)-[1,1′-biphenyl]-4-carboxamide;
N-(6-(2H-1,2,3-triazol-2-yl)-5-(trifluoromethyl)pyridin-3-yl)-5-chloro-2′-(dimethylamino)-2-fluoro-[1,1′-biphenyl]-4-carboxamide;
N-(5-chloro-6-(2H-1,2,3-triazol-2-yl)pyridin-3-yl)-4′,5-dimethyl-[3,3′-bipyridine]-6-carboxamide;
3′-chloro-N4′-(5-chloro-6-(2H-1,2,3-triazol-2-yl)pyridin-3-yl)-[1,1′-biphenyl]-3,4′-dicarboxamide;
2-chloro-N-(5-chloro-6-(2H-1,2,3-triazol-2-yl)pyridin-3-yl)-4-(1-methyl-1H-pyrazol-3-yl)benzamide;
2′-chloro-N-(5-chloro-6-(2H-1,2,3-triazol-2-yl)pyridin-3-yl)-4′-fluoro-[1,1′-biphenyl]-4-carboxamide;
2′-amino-N-(5-chloro-6-(2H-1,2,3-triazol-2-yl)pyridin-3-yl)-[1,1′-biphenyl]-4-carboxamide;
N-(6-(2H-1,2,3-triazol-2-yl)-5-(trifluoromethyl)pyridin-3-yl)-3-chloro-2′-methoxy-[1,1′-biphenyl]-4-carboxamide;
N-(6-(2H-1,2,3-triazol-2-yl)-5-(trifluoromethyl)pyridin-3-yl)-2′-amino-3-fluoro-[1,1′-biphenyl]-4-carboxamide;
N-(6-(2H-1,2,3-triazol-2-yl)-5-(trifluoromethyl)pyridin-3-yl)-2′-acetamido-3-fluoro-[1,1′-biphenyl]-4-carboxamide;
N-(6-(2H-1,2,3-triazol-2-yl)-5-(trifluoromethyl)pyridin-3-yl)-3-fluoro-2′-methoxy-[1,1′-biphenyl]-4-carboxamide;
N-(6-(2H-1,2,3-triazol-2-yl)-5-(trifluoromethyl)pyridin-3-yl)-3,4′-difluoro-2′-hydroxy-[1,1′-biphenyl]-4-carboxamide;
N-(6-(2H-1,2,3-triazol-2-yl)-5-(trifluoromethyl)pyridin-3-yl)-3-fluoro-2′-hydroxy-[1,1′-biphenyl]-4-carboxamide;
N-(6-(2H-1,2,3-triazol-2-yl)-5-(trifluoromethyl)pyridin-3-yl)-3-cyano-4′-fluoro-2′-hydroxy-[1,1′-biphenyl]-4-carboxamide;
N-(6-(2H-1,2,3-triazol-2-yl)-5-(trifluoromethyl)pyridin-3-yl)-3-cyano-2′-hydroxy-[1,1′-biphenyl]-4-carboxamide;
3-chloro-N-(5-chloro-6-(2H-1,2,3-triazol-2-yl)pyridin-3-yl)-4′-fluoro-2′-hydroxy-[1,1′-biphenyl]-4-carboxamide;
3-chloro-N-(5-chloro-6-(2H-1,2,3-triazol-2-yl)pyridin-3-yl)-4′-fluoro-[1,1′-biphenyl]-4-carboxamide;
2-chloro-N-(5-chloro-6-(2H-1,2,3-triazol-2-yl)pyridin-3-yl)-4-(3-chloropyridin-4-yl)benzamide;
3-chloro-N-(5-chloro-6-(2H-1,2,3-triazol-2-yl)pyridin-3-yl)-2′,4′-difluoro-[1,1′-biphenyl]-4-carboxamide;
2′-bromo-3-chloro-N-(5-chloro-6-(2H-1,2,3-triazol-2-yl)pyridin-3-yl)-4′-fluoro-[1,1′-biphenyl]-4-carboxamide;
3-chloro-N-(5-chloro-6-(2H-1,2,3-triazol-2-yl)pyridin-3-yl)-4′-fluoro-2′-(methylthio)-[1,1′-biphenyl]-4-carboxamide;
3-chloro-N-(5-chloro-6-(2H-1,2,3-triazol-2-yl)pyridin-3-yl)-4′-fluoro-2′-methoxy-[1,1′-biphenyl]-4-carboxamide;
N-(6-(2H-1,2,3-triazol-2-yl)-5-(trifluoromethyl)pyridin-3-yl)-2′,5-dichloro-2,4′-difluoro-[1,1′-biphenyl]-4-carboxamide;
N-(6-(2H-1,2,3-triazol-2-yl)-5-(trifluoromethyl)pyridin-3-yl)-2′,5-dichloro-2-fluoro-[1,1′-biphenyl]-4-carboxamide;
2′-amino-5-chloro-N-(5-chloro-6-(2H-1,2,3-triazol-2-yl)pyridin-3-yl)-2-fluoro-[1,1′-biphenyl]-4-carboxamide;
N-(6-(2H-1,2,3-triazol-2-yl)-5-(trifluoromethyl)pyridin-3-yl)-2′-amino-2-chloro-5-fluoro-[1,1′-biphenyl]-4-carboxamide;
N-(6-(2H-1,2,3-triazol-2-yl)-5-(trifluoromethyl)pyridin-3-yl)-2′-amino-2,5-dichloro-[1,1′-biphenyl]-4-carboxamide;
N-(6-(2H-1,2,3-triazol-2-yl)-5-(trifluoromethyl)pyridin-3-yl)-5-chloro-2,4′-difluoro-2′-methoxy-[1,1′-biphenyl]-4-carboxamide;
2′-amino-N-(5-chloro-6-(2H-1,2,3-triazol-2-yl)pyridin-3-yl)-2,4′-difluoro-5-methyl-[1,1′-biphenyl]-4-carboxamide;
2′-bromo-N-(5-chloro-6-(2H-1,2,3-triazol-2-yl)pyridin-3-yl)-2,4′-difluoro-5-methyl-[1,1′-biphenyl]-4-carboxamide;
2′-chloro-N-(5-chloro-6-(2H-1,2,3-triazol-2-yl)pyridin-3-yl)-2,4′-difluoro-5-methyl-[1,1′-biphenyl]-4-carboxamide;
N-(5-chloro-6-(2H-1,2,3-triazol-2-yl)pyridin-3-yl)-2,2′,4′-trifluoro-5-methyl-[1,1′-biphenyl]-4-carboxamide;
N-(5-chloro-6-(2H-1,2,3-triazol-2-yl)pyridin-3-yl)-2-fluoro-2′-methoxy-5-methyl-[1,1′-biphenyl]-4-carboxamide;
N-(5-chloro-6-(2H-1,2,3-triazol-2-yl)pyridin-3-yl)-2,4′-difluoro-2′-methoxy-5-methyl-[1,1′-biphenyl]-4-carboxamide;
2′-amino-2-chloro-N-(5-chloro-6-(2H-1,2,3-triazol-2-yl)pyridin-3-yl)-5-fluoro-[1,1′-biphenyl]-4-carboxamide;
2′-amino-2-chloro-N-(5-chloro-6-(2H-1,2,3-triazol-2-yl)pyridin-3-yl)-4′,5-difluoro-[1,1′-biphenyl]-4-carboxamide;
2′-amino-5-chloro-N-(5-chloro-6-(2H-1,2,3-triazol-2-yl)pyridin-3-yl)-2,4′-difluoro-[1,1′-biphenyl]-4-carboxamide;
N-(6-(2H-1,2,3-triazol-2-yl)-5-(trifluoromethyl)pyridin-3-yl)-2′-amino-5-chloro-2,4′-difluoro-[1,1′-biphenyl]-4-carboxamide;
N-(6-(2H-1,2,3-triazol-2-yl)-5-(trifluoromethyl)pyridin-3-yl)-2′-amino-2-chloro-4′,5-difluoro-[1,1′-biphenyl]-4-carboxamide;
4-(3-aminopyridin-4-yl)-5-chloro-N-(5-chloro-6-(2H-1,2,3-triazol-2-yl)pyridin-3-yl)-2-fluorobenzamide formate;
4-(3-aminopyridin-4-yl)-N-(5-chloro-6-(2H-1,2,3-triazol-2-yl)pyridin-3-yl)-5-fluoro-2-methylbenzamide;
N-(5-chloro-6-(2H-1,2,3-triazol-2-yl)pyridin-3-yl)-4-(3-chloropyridin-4-yl)-5-fluoro-2-methylbenzamide;
2-chloro-N-(5-chloro-6-(2H-1,2,3-triazol-2-yl)pyridin-3-yl)-4′,5-difluoro-2′-(methylamino)-[1,1′-biphenyl]-4-carboxamide;
N-(6-(2H-1,2,3-triazol-2-yl)-5-(trifluoromethyl)pyridin-3-yl)-4-(3-amino-5-fluoropyridin-2-yl)-2-chloro-5-fluorobenzamide;
N-(6-(2H-1,2,3-triazol-2-yl)-5-(trifluoromethyl)pyridin-3-yl)-3-chloro-2′-(difluoromethoxy)-4′-fluoro-[1,1′-biphenyl]-4-carboxamide;
4-(4-aminopyrimidin-5-yl)-2-chloro-N-(5-chloro-6-(2H-1,2,3-triazol-2-yl)pyridin-3-yl)-5-fluorobenzamide;
4-(3-aminopyridazin-4-yl)-2-chloro-N-(5-chloro-6-(2H-1,2,3-triazol-2-yl)pyridin-3-yl)-5-fluorobenzamide;
2-chloro-N-(5-chloro-6-(2H-1,2,3-triazol-2-yl)pyridin-3-yl)-4-(3-chloropyridazin-4-yl)-5-fluorobenzamide;
4-(6-amino-2-oxo-1,2-dihydropyrimidin-5-yl)-2-chloro-N-(5-chloro-6-(2H-1,2,3-triazol-2-yl)pyridin-3-yl)-5-fluorobenzamide;
2′-amino-N-(5-chloro-6-(2H-1,2,3-triazol-2-yl)pyridin-3-yl)-4′,5-difluoro-2-methoxy-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-methoxybenzamide;
2′-bromo-N-(5-chloro-6-(2H-1,2,3-triazol-2-yl)pyridin-3-yl)-4′,5-difluoro-2-methoxy-[1,1′-biphenyl]-4-carboxamide;
N-(6-(2H-1,2,3-triazol-2-yl)-5-(trifluoromethyl)pyridin-3-yl)-3-chloro-4′-fluoro-2′-iodo-[1,1′-biphenyl]-4-carboxamide;
N-(6-(2H-1,2,3-triazol-2-yl)-5-(trifluoromethyl)pyridin-3-yl)-2′,3-dibromo-4′-fluoro-[1,1′-biphenyl]-4-carboxamide;
N-(6-(2H-1,2,3-triazol-2-yl)-5-(trifluoromethyl)pyridin-3-yl)-2′-amino-3-bromo-4′-fluoro-[1,1′-biphenyl]-4-carboxamide;
N-(6-(2H-1,2,3-triazol-2-yl)-5-(trifluoromethyl)pyridin-3-yl)-5-chloro-2,4′-difluoro-2′-iodo-[1,1′-biphenyl]-4-carboxamide;
N-(6-(2H-1,2,3-triazol-2-yl)-5-(trifluoromethyl)pyridin-3-yl)-3-bromo-4′-fluoro-2′-iodo-[1,1′-biphenyl]-4-carboxamide;
N-(6-(2H-1,2,3-triazol-2-yl)-5-(trifluoromethyl)pyridin-3-yl)-2′-amino-5-bromo-2,4′-difluoro-[1,1′-biphenyl]-4-carboxamide;
N-(6-(2H-1,2,3-triazol-2-yl)-5-(trifluoromethyl)pyridin-3-yl)-2-(2-amino-4-fluorophenyl)-4-methylpyrimidine-5-carboxamide;
N-(6-(2H-1,2,3-triazol-2-yl)-5-(trifluoromethyl)pyridin-3-yl)-5-bromo-2,4′-difluoro-2′-iodo-[1,1′-biphenyl]-4-carboxamide;
N-(6-(2H-1,2,3-triazol-2-yl)-5-(trifluoromethyl)pyridin-3-yl)-2′-amino-4′-fluoro-3-iodo-[1,1′-biphenyl]-4-carboxamide;
N-(6-(2H-1,2,3-triazol-2-yl)-5-(trifluoromethyl)pyridin-3-yl)-2′-bromo-5-chloro-2,4′-difluoro-[1,1′-biphenyl]-4-carboxamide;
N-(6-(2H-1,2,3-triazol-2-yl)-5-(trifluoromethyl)pyridin-3-yl)-2,4′-difluoro-2′,5-diiodo-[1,1′-biphenyl]-4-carboxamide;
N-(6-(2H-1,2,3-triazol-2-yl)-5-(trifluoromethyl)pyridin-3-yl)-2′-amino-2,4′-difluoro-5-iodo-[1,1′-biphenyl]-4-carboxamide;
N-(6-(2H-1,2,3-triazol-2-yl)-5-(trifluoromethyl)pyridin-3-yl)-2′-amino-2,4′,5-trifluoro-[1,1′-biphenyl]-4-carboxamide;
N-(6-(2H-1,2,3-triazol-2-yl)-5-(trifluoromethyl)pyridin-3-yl)-2′-chloro-2,4′,5-trifluoro-[1,1′-biphenyl]-4-carboxamide;
N-(6-(2H-1,2,3-triazol-2-yl)-5-(trifluoromethyl)pyridin-3-yl)-4-(3-aminopyridin-4-yl)-2,5-difluorobenzamide;
N-(6-(2H-1,2,3-triazol-2-yl)-5-(trifluoromethyl)pyridin-3-yl)-2′-amino-2-chloro-4′,5-difluoro-[1,1′-biphenyl]-4-carboxamide;
2′-amino-2-bromo-N-(5-chloro-6-(2H-1,2,3-triazol-2-yl)pyridin-3-yl)-4′,5-difluoro-[1,1′-biphenyl]-4-carboxamide;
2′-bromo-2-chloro-N-(5-chloro-6-(2H-1,2,3-triazol-2-yl)pyridin-3-yl)-4′,5-difluoro-[1,1′-biphenyl]-4-carboxamide;
2′-amino-N-(5-chloro-6-(2H-1,2,3-triazol-2-yl)pyridin-3-yl)-4′,5-difluoro-2-iodo-[1,1′-biphenyl]-4-carboxamide;
2-chloro-N-(5-chloro-6-(2H-1,2,3-triazol-2-yl)pyridin-3-yl)-4′,5-difluoro-2′-iodo-[1,1′-biphenyl]-4-carboxamide;
N-(6-(2H-1,2,3-triazol-2-yl)-5-(trifluoromethyl)pyridin-3-yl)-4-(3-bromopyridin-4-yl)-2-chlorobenzamide;
N-(6-(2H-1,2,3-triazol-2-yl)-5-(trifluoromethyl)pyridin-3-yl)-4-(3-aminopyridin-4-yl)-2-bromo-5-fluorobenzamide;
N-(6-(2H-1,2,3-triazol-2-yl)-5-(trifluoromethyl)pyridin-3-yl)-4-(3-bromopyridin-4-yl)-2-chloro-5-fluorobenzamide;
N-(6-(2H-1,2,3-triazol-2-yl)-5-(trifluoromethyl)pyridin-3-yl)-4-(3-aminopyridin-4-yl)-5-fluoro-2-iodobenzamide;
N-(6-(2H-1,2,3-triazol-2-yl)-5-(trifluoromethyl)pyridin-3-yl)-2-chloro-5-fluoro-4-(3-iodopyridin-4-yl)benzamide;
N-(6-(2H-1,2,3-triazol-2-yl)-5-(trifluoromethyl)pyridin-3-yl)-4-(3-aminopyridin-4-yl)-5-fluoro-2-methylbenzamide;
N-(6-(2H-1,2,3-triazol-2-yl)-5-(trifluoromethyl)pyridin-3-yl)-4-(3-aminopyridin-4-yl)-2-chloro-5-fluorobenzamide;
N-(6-(2H-1,2,3-triazol-2-yl)-5-(trifluoromethyl)pyridin-3-yl)-2-chloro-5-fluoro-4-(3-methoxypyridin-4-yl)benzamide;
N-(6-(2H-1,2,3-triazol-2-yl)-5-(trifluoromethyl)pyridin-3-yl)-4-(3-chloropyridin-4-yl)-2,5-difluorobenzamide;
N-(6-(2H-1,2,3-triazol-2-yl)-5-(trifluoromethyl)pyridin-3-yl)-2-chloro-4-(3-chloropyridin-4-yl)-5-fluorobenzamide;
2,2′-dibromo-N-(5-chloro-6-(2H-1,2,3-triazol-2-yl)pyridin-3-yl)-4′,5-difluoro-[1,1′-biphenyl]-4-carboxamide;
4-(3-aminopyridin-4-yl)-5-chloro-N-(5-chloro-6-(2H-1,2,3-triazol-2-yl)pyridin-3-yl)-2-fluorobenzamide;
4-(3-bromopyridin-4-yl)-5-chloro-N-(5-chloro-6-(2H-1,2,3-triazol-2-yl)pyridin-3-yl)-2-fluorobenzamide;
5-chloro-N-(5-chloro-6-(2H-1,2,3-triazol-2-yl)pyridin-3-yl)-4-(3-chloropyridin-4-yl)-2-fluorobenzamide;
(S)-4-(3-aminopyridin-4-yl)-N-(5-chloro-6-(2H-1,2,3-triazol-2-yl)pyridin-3-yl)-2-fluoro-5-(1-methoxyethyl)benzamide;
(S)-4-(3-amino-5-fluoropyridin-2-yl)-N-(5-chloro-6-(2H-1,2,3-triazol-2-yl)pyridin-3-yl)-2-fluoro-5-(1-methoxyethyl)benzamide;
2′-amino-N-(5-chloro-6-(2H-1,2,3-triazol-2-yl)pyridin-3-yl)-4′,5-difluoro-2-isopropoxy-[1,1′-biphenyl]-4-carboxamide;
2′-bromo-N-(5-chloro-6-(2H-1,2,3-triazol-2-yl)pyridin-3-yl)-4′,5-difluoro-2-isopropoxy-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-methylbenzamide;
4-(3-aminopyridin-4-yl)-N-(5-chloro-6-(2H-1,2,3-triazol-2-yl)pyridin-3-yl)-2-fluoro-5-isopropylbenzamide;
2′-amino-N-(5-chloro-6-(2H-1,2,3-triazol-2-yl)pyridin-3-yl)-4′,5-difluoro-2-isobutyl-[1,1′-biphenyl]-4-carboxamide;
2′-bromo-N-(5-chloro-6-(2H-1,2,3-triazol-2-yl)pyridin-3-yl)-4′,5-difluoro-2-isobutyl-[1,1′-biphenyl]-4-carboxamide;
4-(3-aminopyridin-4-yl)-N-(5-chloro-6-(2H-1,2,3-triazol-2-yl)pyridin-3-yl)-5-cyclopropyl-2-fluorobenzamide;
4-(3-aminopyridin-4-yl)-N-(5-chloro-6-(2H-1,2,3-triazol-2-yl)pyridin-3-yl)-2-fluoro-5-isopropoxybenzamide;
2′-amino-N-(5-chloro-6-(2H-1,2,3-triazol-2-yl)pyridin-3-yl)-2-cyclopropyl-4′,5-difluoro-[1,1′-biphenyl]-4-carboxamide;
2′-bromo-N-(5-chloro-6-(2H-1,2,3-triazol-2-yl)pyridin-3-yl)-2-cyclopropyl-4′,5-difluoro-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-(tetrahydro-2H-pyran-4-yl)benzamide;
4-(3-aminopyridin-4-yl)-N-(5-chloro-6-(2H-1,2,3-triazol-2-yl)pyridin-3-yl)-5-ethynyl-2-fluorobenzamide;
2′-amino-N-(5-chloro-6-(2H-1,2,3-triazol-2-yl)pyridin-3-yl)-2-ethynyl-4′,5-difluoro-[1,1′-biphenyl]-4-carboxamide;
2′-bromo-N-(5-chloro-6-(2H-1,2,3-triazol-2-yl)pyridin-3-yl)-2-ethynyl-4′,5-difluoro-[1,1′-biphenyl]-4-carboxamide;
4-(3-aminopyridin-4-yl)-5-(tert-butyl)-N-(5-chloro-6-(2H-1,2,3-triazol-2-yl)pyridin-3-yl)-2-fluorobenzamide;
4-(3-aminopyridin-4-yl)-N-(5-chloro-6-(2H-1,2,3-triazol-2-yl)pyridin-3-yl)-2-fluoro-5-isobutylbenzamide;
2′-bromo-N-(5-chloro-6-(2H-1,2,3-triazol-2-yl)pyridin-3-yl)-4′,5-difluoro-2-(oxetan-3-yl)-[1,1′-biphenyl]-4-carboxamide;
4-(3-aminopyridin-4-yl)-N-(5-chloro-6-(2H-1,2,3-triazol-2-yl)pyridin-3-yl)-2-fluoro-5-(oxetan-3-yl)benzamide;
4-(3-aminopyridin-4-yl)-N-(5-chloro-6-(2H-1,2,3-triazol-2-yl)pyridin-3-yl)-5-ethyl-2-fluorobenzamide;
2′-amino-N-(5-chloro-6-(2H-1,2,3-triazol-2-yl)pyridin-3-yl)-4′,5-difluoro-2-isopropyl-[1,1′-biphenyl]-4-carboxamide;
2′-bromo-N-(5-chloro-6-(2H-1,2,3-triazol-2-yl)pyridin-3-yl)-4′,5-difluoro-2-isopropyl-[1,1′-biphenyl]-4-carboxamide;
5-chloro-N-(5-chloro-6-(2H-1,2,3-triazol-2-yl)pyridin-3-yl)-2′-(difluoromethyl)-2,4′-difluoro-[1,1′-biphenyl]-4-carboxamide;
5-chloro-N-(5-chloro-6-(2H-1,2,3-triazol-2-yl)pyridin-3-yl)-2′-(difluoromethoxy)-2,4′-difluoro-[1,1′-biphenyl]-4-carboxamide;
2-chloro-N-(5-chloro-6-(2H-1,2,3-triazol-2-yl)pyridin-3-yl)-5-fluoro-4-(6-oxo-1,6-dihydropyridin-3-yl)benzamide;
2-chloro-N-(5-chloro-6-(2H-1,2,3-triazol-2-yl)pyridin-3-yl)-5-fluoro-4-(1-methyl-6-oxo-1,6-dihydropyridin-3-yl)benzamide;
2-chloro-N-(5-chloro-6-(2H-1,2,3-triazol-2-yl)pyridin-3-yl)-5-fluoro-4-(5-fluoro-1-methyl-6-oxo-1,6-dihydropyridin-3-yl)benzamide;
N-(5-chloro-6-(2H-1,2,3-triazol-2-yl)pyridin-3-yl)-2,4′-difluoro-2′-hydroxy-5-methyl-[1,1′-biphenyl]-4-carboxamide;
N-(5-chloro-6-(2H-1,2,3-triazol-2-yl)pyridin-3-yl)-5-fluoro-4-(3-hydroxypyridin-4-yl)-2-methylbenzamide;
4-(5-aminopyrimidin-4-yl)-N-(5-chloro-6-(2H-1,2,3-triazol-2-yl)pyridin-3-yl)-5-fluoro-2-methylbenzamide;
2-chloro-N-(5-chloro-6-(2H-1,2,3-triazol-2-yl)pyridin-3-yl)-5-fluoro-4-(5-fluoro-3-methylpyridin-2-yl)benzamide;
5-chloro-N-(5-chloro-6-(2H-1,2,3-triazol-2-yl)pyridin-3-yl)-2′-(difluoromethyl)-2,4′-difluoro-[1,1′-biphenyl]-4-carboxamide;
2-chloro-N-(5-chloro-6-(2H-1,2,3-triazol-2-yl)pyridin-3-yl)-5-fluoro-4-(6-oxo-1,6-dihydropyridin-3-yl)benzamide;
N-(5-chloro-6-(2H-1,2,3-triazol-2-yl)pyridin-3-yl)-2,4′-difluoro-2′-hydroxy-5-methyl-[1,1′-biphenyl]-4-carboxamide;
5-chloro-N-(5-chloro-6-(2H-1,2,3-triazol-2-yl)pyridin-3-yl)-2′-cyano-2,4′-difluoro-[1,1′-biphenyl]-4-carboxamide;
5-chloro-N-(5-chloro-6-(2H-1,2,3-triazol-2-yl)pyridin-3-yl)-2′-(difluoromethoxy)-2,4′-difluoro-[1,1′-biphenyl]-4-carboxamide;
2-chloro-N-(5-chloro-6-(2H-1,2,3-triazol-2-yl)pyridin-3-yl)-5-fluoro-4-(1-methyl-6-oxo-1,6-dihydropyridin-3-yl)benzamide;
N-(5-chloro-6-(2H-1,2,3-triazol-2-yl)pyridin-3-yl)-5-fluoro-4-(3-hydroxypyridin-4-yl)-2-methylbenzamide;
2-chloro-N-(5-chloro-6-(2H-1,2,3-triazol-2-yl)pyridin-3-yl)-4-(3-cyanopyridin-4-yl)-5-fluorobenzamide;
N-(6-(2H-1,2,3-triazol-2-yl)-5-(trifluoromethyl)pyridin-3-yl)-2′-amino-5-chloro-4′-fluoro-2-(trifluoromethyl)-[1,1′-biphenyl]-4-carboxamide;
N-(6-(2H-1,2,3-triazol-2-yl)-5-(trifluoromethyl)pyridin-3-yl)-2,2′,3,4′,5,6-hexafluoro-[1,1′-biphenyl]-4-carboxamide;
2′-chloro-N-(5-chloro-6-(2H-1,2,3-triazol-2-yl)pyridin-3-yl)-2,3,4′,5,6-pentafluoro-[1,1′-biphenyl]-4-carboxamide;
N-(6-(2H-1,2,3-triazol-2-yl)-5-(trifluoromethyl)pyridin-3-yl)-2′,5-dichloro-4′-fluoro-2-(trifluoromethyl)-[1,1′-biphenyl]-4-carboxamide;
N-(6-(2H-1,2,3-triazol-2-yl)-5-(trifluoromethyl)pyridin-3-yl)-5-chloro-2′,4′-difluoro-2-(trifluoromethyl)-[1,1′-biphenyl]-4-carboxamide;
2′-amino-5-chloro-N-(5-chloro-6-(2H-1,2,3-triazol-2-yl)pyridin-3-yl)-2-(trifluoromethyl)-[1,1′-biphenyl]-4-carboxamide;
5-chloro-N-(5-chloro-6-(2H-1,2,3-triazol-2-yl)pyridin-3-yl)-2,2′,4′-trifluoro-[1,1′-biphenyl]-4-carboxamide;
2′-bromo-5-chloro-N-(5-chloro-6-(2H-1,2,3-triazol-2-yl)pyridin-3-yl)-2,4′-difluoro-[1,1′-biphenyl]-4-carboxamide;
2′-amino-5-chloro-N-(5-chloro-6-(2H-1,2,3-triazol-2-yl)pyridin-3-yl)-4′-fluoro-2-(trifluoromethyl)-[1,1′-biphenyl]-4-carboxamide;
2′-amino-5-chloro-N-(5-chloro-6-(2H-1,2,3-triazol-2-yl)pyridin-3-yl)-2-cyclopropyl-4′-fluoro-[1,1′-biphenyl]-4-carboxamide;
5-chloro-N-(5-chloro-6-(2H-1,2,3-triazol-2-yl)pyridin-3-yl)-2′-(difluoromethyl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(trifluoromethyl)-[1,1′-biphenyl]-4-carboxamide;
5-chloro-N-(5-chloro-6-(2H-1,2,3-triazol-2-yl)pyridin-3-yl)-2′-cyclopropyl-2,4′-difluoro-[1,1′-biphenyl]-4-carboxamide;
2′-amino-5-chloro-N-(5-chloro-6-(2H-1,2,3-triazol-2-yl)pyridin-3-yl)-4′-fluoro-2-methyl-[1,1′-biphenyl]-4-carboxamide;
4-(3-aminopyridin-4-yl)-2-chloro-N-(5-chloro-6-(2H-1,2,3-triazol-2-yl)pyridin-3-yl)-5-(trifluoromethyl)benzamide;
2-chloro-N-(5-chloro-6-(2H-1,2,3-triazol-2-yl)pyridin-3-yl)-4-(3-chloropyridin-4-yl)-5-(trifluoromethyl)benzamide;
4-(3-bromopyridin-4-yl)-2-chloro-N-(5-chloro-6-(2H-1,2,3-triazol-2-yl)pyridin-3-yl)-5-(trifluoromethyl)benzamide;
2′-amino-5-chloro-N-(5-chloro-6-(2H-1,2,3-triazol-2-yl)pyridin-3-yl)-2-ethynyl-4′-fluoro-[1,1′-biphenyl]-4-carboxamide;
2′,5-dichloro-N-(5-chloro-6-(2H-1,2,3-triazol-2-yl)pyridin-3-yl)-4′-fluoro-2-(trifluoromethyl)-[1,1′-biphenyl]-4-carboxamide;
2′-bromo-5-chloro-N-(5-chloro-6-(2H-1,2,3-triazol-2-yl)pyridin-3-yl)-4′-fluoro-2-(trifluoromethyl)-[1,1′-biphenyl]-4-carboxamide;
5-chloro-N-(5-chloro-6-(2H-1,2,3-triazol-2-yl)pyridin-3-yl)-4′-fluoro-2′-methyl-2-(trifluoromethyl)-[1,1′-biphenyl]-4-carboxamide;
2′-amino-2,5-dichloro-N-(5-chloro-6-(2H-1,2,3-triazol-2-yl)pyridin-3-yl)-4′-fluoro-[1,1′-biphenyl]-4-carboxamide;
2′,5-dichloro-2,4′-difluoro-N-(6-methyl-5-oxo-5,6-dihydro-1,6-naphthyridin-3-yl)-[1,1′-biphenyl]-4-carboxamide;
2′,5-dichloro-N-(7-chloro-2-methylbenzo[d]oxazol-5-yl)-2,4′-difluoro-[1,1′-biphenyl]-4-carboxamide;
2′,5-dichloro-N-(8-chloro-3-oxo-3,4-dihydro-2H-benzo[b][1,4]oxazin-6-yl)-2,4′-difluoro-[1,1′-biphenyl]-4-carboxamide;
2′,5-dichloro-2,4′-difluoro-N-(1-methyl-1H-pyrazolo[4,3-b]pyridin-6-yl)-[1,1′-biphenyl]-4-carboxamide;
2′,5-dichloro-2,4′-difluoro-N-(2-methyl-2H-pyrazolo[4,3-b]pyridin-6-yl)-[1,1′-biphenyl]-4-carboxamide;
2′,5-dichloro-2,4′-difluoro-N-(1-methyl-1H-pyrazolo[3,4-b]pyridin-5-yl)-[1,1′-biphenyl]-4-carboxamide;
2′,5-dichloro-N-(2-chloropyrazolo[1,5-a]pyrimidin-6-yl)-2,4′-difluoro-[1,1′-biphenyl]-4-carboxamide;
2′,5-dichloro-2,4′-difluoro-N-(2-methylimidazo[1,2-b]pyridazin-7-yl)-[1,1′-biphenyl]-4-carboxamide;
2′,5-dichloro-N-(2-chlorothiazolo[5,4-b]pyridin-6-yl)-2,4′-difluoro-[1,1′-biphenyl]-4-carboxamide;
2′,5-dichloro-N-(6-chloro-1,5-naphthyridin-3-yl)-2,4′-difluoro-[1,1′-biphenyl]-4-carboxamide;
2′,5-dichloro-2,4′-difluoro-N-(2-(trifluoromethyl)pyridin-4-yl)-[1,1′-biphenyl]-4-carboxamide;
2′,5-dichloro-N-(2-cyanopyridin-4-yl)-2,4′-difluoro-[1,1′-biphenyl]-4-carboxamide;
2′,5-dichloro-N-(2-chloropyridin-4-yl)-2,4′-difluoro-[1,1′-biphenyl]-4-carboxamide;
2′,5-dichloro-N-(5-chloro-6-methoxypyridin-3-yl)-2,4′-difluoro-[1,1′-biphenyl]-4-carboxamide;
2′,5-dichloro-N-(6-cyano-5-(trifluoromethyl)pyridin-3-yl)-2,4′-difluoro-[1,1′-biphenyl]-4-carboxamide;
2′,5-dichloro-N-(6-cyano-5-fluoropyridin-3-yl)-2,4′-difluoro-[1,1′-biphenyl]-4-carboxamide;
2′,5-dichloro-2,4′-difluoro-N-(2-methyl-6-(trifluoromethyl)pyridin-4-yl)-[1,1′-biphenyl]-4-carboxamide;
2′,5-dichloro-N-(5-chloro-6-(1-methyl-1H-pyrazol-4-yl)pyridin-3-yl)-2,4′-difluoro-[1,1′-biphenyl]-4-carboxamide;
2′,5-dichloro-N-(5-chloro-6-(3-methyl-1H-1,2,4-triazol-1-yl)pyridin-3-yl)-2,4′-difluoro-[1,1′-biphenyl]-4-carboxamide;
2′,5-dichloro-N-(5-chloro-6-(oxazol-2-yl)pyridin-3-yl)-2,4′-difluoro-[1,1′-biphenyl]-4-carboxamide;
2′,5-dichloro-N-(5-chloro-6-(thiazol-2-yl)pyridin-3-yl)-2,4′-difluoro-[1,1′-biphenyl]-4-carboxamide;
2′,5-dichloro-N-(5-chloro-6-(1H-pyrazol-1-yl)pyridin-3-yl)-2,4′-difluoro-[1,1′-biphenyl]-4-carboxamide;
2′,5-dichloro-N-(5-cyano-6-(2H-1,2,3-triazol-2-yl)pyridin-3-yl)-2,4′-difluoro-[1,1′-biphenyl]-4-carboxamide;
2′,5-dichloro-N-(5-cyano-6-cyclopropoxypyridin-3-yl)-2,4′-difluoro-[1,1′-biphenyl]-4-carboxamide;
2′,5-dichloro-N-(5-cyano-6-(difluoromethoxy)pyridin-3-yl)-2,4′-difluoro-[1,1′-biphenyl]-4-carboxamide;
4-chloro-6-(2-chloro-4-fluorophenyl)-N-(5-chloro-6-(2H-1,2,3-triazol-2-yl)pyridin-3-yl)nicotinamide;
6-(2-amino-4-fluorophenyl)-4-chloro-N-(5-chloro-6-(2H-1,2,3-triazol-2-yl)pyridin-3-yl)nicotinamide;
2-(2-amino-4-fluorophenyl)-4-chloro-N-(5-chloro-6-(2H-1,2,3-triazol-2-yl)pyridin-3-yl)pyrimidine-5-carboxamide;
6-(2-amino-4-fluorophenyl)-2-chloro-N-(5-chloro-6-(2H-1,2,3-triazol-2-yl)pyridin-3-yl)nicotinamide;
4-chloro-6-(2-chloro-4-fluorophenyl)-N-(5-chloro-6-(2H-1,2,3-triazol-2-yl)pyridin-3-yl)pyridazine-3-carboxamide;
3-chloro-5-(2-chloro-4-fluorophenyl)-N-(5-chloro-6-(2H-1,2,3-triazol-2-yl)pyridin-3-yl)pyrazine-2-carboxamide;
N-(6-(2H-1,2,3-triazol-2-yl)-5-(trifluoromethyl)pyridin-3-yl)-4-(3-bromopyridin-4-yl)-2-chloro-5-(trifluoromethyl)benzamide;
2-amino-2′,5-dichloro-N-(5-chloro-6-(2H-1,2,3-triazol-2-yl)pyridin-3-yl)-4′-fluoro-[1,1′-biphenyl]-4-carboxamide;
2-acetamido-2′,5-dichloro-N-(5-chloro-6-(2H-1,2,3-triazol-2-yl)pyridin-3-yl)-4′-fluoro-[1,1′-biphenyl]-4-carboxamide;
ethyl(2′,5-dichloro-4-((5-chloro-6-(2H-1,2,3-triazol-2-yl)pyridin-3-yl)aminoformyl)-4′-fluoro-[1,1′-biphenyl]-2-yl)aminoformate;
2′,5-dichloro-N-(5-chloro-6-(2H-1,2,3-triazol-2-yl)pyridin-3-yl)-4′-fluoro-2-(isopropylamino)-[1,1′-biphenyl]-4-carboxamide;
N-(6-(2H-1,2,3-triazol-2-yl)-5-(trifluoromethyl)pyridin-3-yl)-5-chloro-2,2′,4′-trifluoro-[1,1′-biphenyl]-4-carboxamide;
5-chloro-N-(5-chloro-6-(2H-1,2,3-triazol-2-yl)pyridin-3-yl)-2,2′,4′-trifluoro-[1,1′-biphenyl]-4-carboxamide;
N-(5-chloro-6-(2H-1,2,3-triazol-2-yl)pyridin-3-yl)-2,2′,4′,5-tetrafluoro-[1,1′-biphenyl]-4-carboxamide;
N-(6-(2H-1,2,3-triazol-2-yl)-5-(trifluoromethyl)pyridin-3-yl)-2,2′,4′,5-tetrafluoro-[1,1′-biphenyl]-4-carboxamide;
4-(3-bromopyridin-4-yl)-2-chloro-N-(5-chloro-6-(2H-1,2,3-triazol-2-yl)pyridin-3-yl)-5-fluorobenzamide;
4-(2-amino-6-fluoropyridin-3-yl)-N-(5-chloro-6-(2H-1,2,3-triazol-2-yl)pyridin-3-yl)-2-Fluoro-5-isopropylbenzamide;
5-chloro-N-(5-chloro-6-(2H-1,2,3-triazol-2-yl)pyridin-3-yl)-2′-(cyclopropylamino)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(methylthio)-[1,1′-biphenyl]-4-carboxamide;
5-chloro-N-(5-chloro-6-(2H-1,2,3-triazol-2-yl)pyridin-3-yl)-2,4′-difluoro-2′-(methylsulfonyl)-[1,1′-biphenyl]-4-carboxamide;
5-chloro-N-(5-chloro-6-(2H-1,2,3-triazol-2-yl)pyridin-3-yl)-2,4′-difluoro-2′-ureido-[1,1′-biphenyl]-4-carboxamide;
2′-acetamido-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(hydroxymethyl)-[1,1′-biphenyl]-4-carboxamide;
4-(3-amino-5-ethynylpyridin-4-yl)-2-chloro-N-(5-chloro-6-(2H-1,2,3-triazol-2-yl)pyridin-3-yl)-5-fluorobenzamide;
5-chloro-N-(5-chloro-6-(2H-1,2,3-triazol-2-yl)pyridin-3-yl)-2,4′-difluoro-2′-(methylsulfinyl)-[1,1′-biphenyl]-4-carboxamide;
5-chloro-N-(5-chloro-6-(2H-1,2,3-triazol-2-yl)pyridin-3-yl)-2,4′-difluoro-2′-((methylsulfonyl)methyl)-[1,1′-biphenyl]-4-carboxamide;
ethyl (5′-chloro-4′-((5-chloro-6-(2H-1,2,3-triazol-2-yl)pyridin-3-yl)carbamoyl)-2′,4-difluoro-[1,1′-biphenyl]-2-yl)carbamate;
5-chloro-N-(5-chloro-6-(2H-1,2,3-triazol-2-yl)pyridin-3-yl)-2,4′-difluoro-2′-vinyl-[1,1′-biphenyl]-4-carboxamide;
5-chloro-N-(5-chloro-6-(2H-1,2,3-triazol-2-yl)pyridin-3-yl)-2,4′-difluoro-2′-(1-hydroxyethyl)-[1,1′-biphenyl]-4-carboxamide
5-chloro-N-(5-chloro-6-(2H-1,2,3-triazol-2-yl)pyridin-3-yl)-2,4′-difluoro-2′-(1-methoxyethyl)-[1,1′-biphenyl]-4-carboxamide;
5-chloro-N-(5-chloro-6-(2H-1,2,3-triazol-2-yl)pyridin-3-yl)-2,4′-difluoro-2′-((2-(methylamino)ethyl)amino)-[1,1′-biphenyl]-4-carboxamide;
5-chloro-N-(5-chloro-6-(2H-1,2,3-triazol-2-yl)pyridin-3-yl)-2,4′-difluoro-2′-((2-hydroxyethyl)amino)-[1,1′-biphenyl]-4-carboxamide;
5-chloro-N-(5-chloro-6-(2H-1,2,3-triazol-2-yl)pyridin-3-yl)-2,4′-difluoro-2′-(2-hydroxyethoxy)-[1,1′-biphenyl]-4-carboxamide;
5-chloro-N-(5-chloro-6-(2H-1,2,3-triazol-2-yl)pyridin-3-yl)-2,4′-difluoro-2′-(hydroxyamino)-[1,1′-biphenyl]-4-carboxamide;
5-chloro-N-(5-chloro-6-(2H-1,2,3-triazol-2-yl)pyridin-3-yl)-2,4′-difluoro-2′-(2-hydroxypropan-2-yl)-[1,1′-biphenyl]-4-carboxamide;
5′-chloro-4′-((5-chloro-6-(2H-1,2,3-triazol-2-yl)pyridin-3-yl)carbamoyl)-2′,4-difluoro-[1,1′-biphenyl]-2-yl ethyl carbonate;
5-chloro-N-(5-chloro-6-(2H-1,2,3-triazol-2-yl)pyridin-3-yl)-2′-((2-(dimethylamino)ethyl)amino)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-((2-methoxyethyl)amino)-[1,1′-biphenyl]-4-carboxamide;
5-chloro-N-(5-chloro-6-(2H-1,2,3-triazol-2-yl)pyridin-3-yl)-2,4′-difluoro-2′-(2-methoxyethoxy)-[1,1′-biphenyl]-4-carboxamide;
5-chloro-N-(5-chloro-6-(2H-1,2,3-triazol-2-yl)pyridin-3-yl)-2,4′-difluoro-2′-(prop-2-yn-1-ylamino)-[1,1′-biphenyl]-4-carboxamide;
5-chloro-N-(5-chloro-6-(2H-1,2,3-triazol-2-yl)pyridin-3-yl)-2′-((cyanomethyl)amino)-2,4′-difluoro-[1,1′-biphenyl]-4-carboxamide;
2′-(aminomethyl)-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(prop-2-yn-1-yl)-[1,1′-biphenyl]-4-carboxamide;
2′-acrylamido-5-chloro-N-(5-chloro-6-(2H-1,2,3-triazol-2-yl)pyridin-3-yl)-2,4′-difluoro-[1,1′-biphenyl]-4-carboxamide;
5′-chloro-N4′-(5-chloro-6-(2H-1,2,3-triazol-2-yl)pyridin-3-yl)-2′,4-difluoro-[1,1′-biphenyl]-2,4′-dicarboxamide;
5-chloro-N-(5-chloro-6-(2H-1,2,3-triazol-2-yl)pyridin-3-yl)-2,4′-difluoro-2′-(prop-2-yn-1-yloxy)-[1,1′-biphenyl]-4-carboxamide;
5-chloro-N-(5-chloro-6-(2H-1,2,3-triazol-2-yl)pyridin-3-yl)-2′-(cyanomethoxy)-2,4′-difluoro-[1,1′-biphenyl]-4-carboxamide;
2′-(1-aminoethyl)-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2′-(cyanomethyl)-2,4′-difluoro-[1,1′-biphenyl]-4-carboxamide;
2′-amino-5-chloro-N-(5-chloro-6-(2H-1,2,3-triazol-2-yl)pyridin-3-yl)-6′-ethynyl-2,4′-difluoro-[1,1′-biphenyl]-4-carboxamide;
cyclopropyl 5′-chloro-4′-((5-chloro-6-(2H-1,2,3-triazol-2-yl)pyridin-3-yl)carbamoyl)-2′,4-difluoro-[1,1′-biphenyl]-2-carboxylate;
2′,5-dichloro-N-(5-chloro-6-(2H-1,2,3-triazol-2-yl)pyridin-3-yl)-6′-ethynyl-2,4′-difluoro-[1,1′-biphenyl]-4-carboxamide;
2′-(2-aminopropan-2-yl)-5-chloro-N-(5-chloro-6-(2H-1,2,3-triazol-2-yl)pyridin-3-yl)-2,4′-difluoro-[1,1′-biphenyl]-4-carboxamide;
5-chloro-N-(5-chloro-6-(2H-1,2,3-triazol-2-yl)pyridin-3-yl)-2,4′-difluoro-2′-(methoxymethyl)-[1,1′-biphenyl]-4-carboxamide;
5′-chloro-4′-((5-chloro-6-(2H-1,2,3-triazol-2-yl)pyridin-3-yl)carbamoyl)-2′,4-difluoro-[1,1′-biphenyl]-2-yl carbamate;
2′,5-dichloro-N-(5-chloro-6-(2H-1,2,3-triazol-2-yl)pyridin-3-yl)-2,4′-difluoro-6′-(methoxymethyl)-[1,1′-biphenyl]-4-carboxamide;
N-(6-(2H-1,2,3-triazol-2-yl)-5-(trifluoromethyl)pyridin-3-yl)-5-chloro-2,4′-difluoro-2′-vinyl-[1,1′-biphenyl]-4-carboxamide;
N-(6-(2H-1,2,3-triazol-2-yl)-5-(trifluoromethyl)pyridin-3-yl)-5-chloro-2′-ethynyl-2,4′-difluoro-[1,1′-biphenyl]-4-carboxamide;
2-chloro-N-(5-chloro-6-(2H-1,2,3-triazol-2-yl)pyridin-3-yl)-4-(2-ethynyl-6-fluoropyridin-3-yl)-5-fluorobenzamide;
2-chloro-N-(5-chloro-6-(2H-1,2,3-triazol-2-yl)pyridin-3-yl)-4-(3-ethynylpyridin-4-yl)-5-fluorobenzamide;
5-chloro-2′-ethynyl-2,4′-difluoro-N-(2-(trifluoromethyl)pyridin-4-yl)-[1,1′-biphenyl]-4-carboxamide;
5-chloro-N-(8-chloro-3-oxo-3,4-dihydro-2H-benzo[b][1,4]oxazin-6-yl)-2′-ethynyl-2,4′-difluoro-[1,1′-biphenyl]-4-carboxamide;
5-chloro-N-(6-cyano-5-(trifluoromethyl)pyridin-3-yl)-2′-ethynyl-2,4′-difluoro-[1,1′-biphenyl]-4-carboxamide;
N-(6-(2H-1,2,3-triazol-2-yl)-5-(trifluoromethyl)pyridin-3-yl)-2-chloro-4-(2-ethynyl-6-fluoropyridin-3-yl)-5-fluorobenzamide;
2-chloro-4-(3-ethynylpyridin-4-yl)-5-fluoro-N-(6-((tetrahydrofuran-3-yl)amino)-5-(trifluoromethyl)pyridin-3-yl)benzamide;
2-chloro-4-(3-ethynyl-5-fluoropyridin-2-yl)-5-fluoro-N-(2-(trifluoromethyl)pyridin-4-yl)benzamide;
2-chloro-N-(8-chloro-3-oxo-3,4-dihydro-2H-benzo[b][1,4]oxazin-6-yl)-4-(3-ethynyl-5-fluoropyridin-2-yl)-5-fluorobenzamide;
2-chloro-N-(6-cyano-5-(trifluoromethyl)pyridin-3-yl)-4-(3-ethynyl-5-fluoropyridin-2-yl)-5-fluorobenzamide;
N-(6-(2H-1,2,3-triazol-2-yl)-5-(trifluoromethyl)pyridin-3-yl)-2-chloro-4-(3-ethynyl-5-fluoropyridin-2-yl)-5-fluorobenzamide;
5-chloro-N-(5-cyano-6-(trifluoromethoxy)pyridin-3-yl)-2′-ethynyl-2,4′-difluoro-[1,1′-biphenyl]-4-carboxamide;
5-chloro-N-(5-chloro-6-(difluoromethoxy)pyridin-3-yl)-2′-ethynyl-2,4′-difluoro-[1,1′-biphenyl]-4-carboxamide;
5-chloro-N-(5-cyano-6-((1-methylpiperidin-4-yl)oxy)pyridin-3-yl)-2′-ethynyl-2,4′-difluoro-[1,1′-biphenyl]-4-carboxamide;
N-(6-(2H-1,2,3-triazol-2-yl)-5-(trifluoromethyl)pyridin-3-yl)-2-chloro-4-(3-ethynylpyridin-4-yl)-5-fluorobenzamide;
N-(5-chloro-6-(2H-1,2,3-triazol-2-yl)pyridin-3-yl)-2,3,4′,5,6-pentafluoro-2′-propiolamido-[1,1′-biphenyl]-4-carboxamide;
N-(5-chloro-6-(2H-1,2,3-triazol-2-yl)pyridin-3-yl)-2,3,4′,5,6-pentafluoro-2′-propiolamido-[1,1′-biphenyl]-4-carboxamide;
N-(5-chloro-6-(2H-1,2,3-triazol-2-yl)pyridin-3-yl)-4-(3-ethynylpyridin-4-yl)-2,3,5,6-tetrafluorobenzamide;
2-chloro-N-(3-chloro-4-(2-methoxyethoxy)phenyl)-4-(3-ethynylpyridin-4-yl)-5-fluorobenzamide;
2-chloro-N-(5-chloro-6-(2H-1,2,3-triazol-2-yl)pyridin-3-yl)-4-(3-ethynyl-5-fluoropyridin-2-yl)-5-fluorobenzamide;
6-chloro-N-(5-chloro-6-(2H-1,2,3-triazol-2-yl)pyridin-3-yl)-3′-ethynyl-[2,4′-bipyridine]-5-carboxamide;
2-chloro-N-(5-chloro-6-(2H-1,2,3-triazol-2-yl)pyridin-3-yl)-5-fluoro-4-(3-propiolamidopyridin-4-yl)benzamide;
2-chloro-N-(3-chloro-4-methoxyphenyl)-4-(3-ethynylpyridin-4-yl)-5-fluorobenzamide;
2-chloro-4-(3-ethynyl-5-fluoropyridin-2-yl)-5-fluoro-N-(6-((tetrahydrofuran-3-yl)amino)-5-(trifluoromethyl)pyridin-3-yl)benzamide;
N-(5-chloro-6-(2H-1,2,3-triazol-2-yl)pyridin-3-yl)-4-(3-ethynyl-5-fluoropyridin-2-yl)-2,3,5,6-tetrafluorobenzamide;
N-(5-chloro-6-(2H-1,2,3-triazol-2-yl)pyridin-3-yl)-2,3,5,6-tetrafluoro-4-(5-fluoro-3-propiolamidopyridin-2-yl)benzamide;
4-(3-acrylamidopyridin-4-yl)-2-chloro-N-(5-chloro-6-(2H-1,2,3-triazol-2-yl)pyridin-3-yl)-5-fluorobenzamide;
N-(3-chloro-4-(methylsulfonyl)phenyl)-4-(3-ethynylpyridin-4-yl)-2,3,5,6-tetrafluorobenzamide;
N-(3-chloro-4-((trifluoromethyl)thio)phenyl)-4-(3-ethynylpyridin-4-yl)-2,3,5,6-tetrafluorobenzamide;
N-(3-chloro-4-((2-(dimethylamino)-2-oxoethyl)thio)phenyl)-4-(3-ethynylpyridin-4-yl)-2,3,5,6-tetrafluorobenzamide;
N-(6-(2H-1,2,3-triazol-2-yl)-5-(trifluoromethyl)pyridin-3-yl)-2-chloro-5-fluoro-4-(3-vinylpyridin-4-yl)benzamide;
2′-amino-5-chloro-2,4′-difluoro-N-(6-(2-methoxyethoxy)-5-(trifluoromethyl)pyridin-3-yl)-[1,1′-biphenyl]-4-carboxamide;
2′-amino-5-chloro-N-(6-(cyclopropylamino)-5-(trifluoromethyl)pyridin-3-yl)-2,4′-difluoro-[1,1′-biphenyl]-4-carboxamide;
2′-amino-5-chloro-N-(6-(cyanomethoxy)-5-(trifluoromethyl)pyridin-3-yl)-2,4′-difluoro-[1,1′-biphenyl]-4-carboxamide;
2′-amino-5-chloro-N-(6-((cyanomethyl)amino)-5-(trifluoromethyl)pyridin-3-yl)-2,4′-difluoro-[1,1′-biphenyl]-4-carboxamide;
2′-amino-5-chloro-2,4′-difluoro-N-(6-(methoxymethyl)-5-(trifluoromethyl)pyridin-3-yl)-[1,1′-biphenyl]-4-carboxamide;
2′-amino-5-chloro-2,4′-difluoro-N-(6-((2-methoxyethyl)amino)-5-(trifluoromethyl)pyridin-3-yl)-[1,1′-biphenyl]-4-carboxamide;
methyl (4-(2′-amino-5-chloro-2,4′-difluoro-[1,1′-biphenyl]-4-carboxamido)-2-(trifluoromethyl)phenyl)carbamate;
N-(4-acetamido-3-(trifluoromethyl)phenyl)-2′-amino-5-chloro-2,4′-difluoro-[1,1′-biphenyl]-4-carboxamide;
2′-amino-5-chloro-2,4′-difluoro-N-(3-(trifluoromethyl)-4-ureidophenyl)-[1,1′-biphenyl]-4-carboxamide;
2′-amino-5-chloro-2,4′-difluoro-N-(4-(methylsulfonamido)-3-(trifluoromethyl)phenyl)-[1,1′-biphenyl]-4-carboxamide;
2′-amino-5-chloro-2,4′-difluoro-N-(6-(hydroxymethyl)-5-(trifluoromethyl)pyridin-3-yl)-[1,1′-biphenyl]-4-carboxamide;
2′-amino-5-chloro-N-(6-(cyanomethyl)-5-(trifluoromethyl)pyridin-3-yl)-2,4′-difluoro-[1,1′-biphenyl]-4-carboxamide;
2′-amino-5-chloro-2,4′-difluoro-N-(4-(methylthio)-3-(trifluoromethyl)phenyl)-[1,1′-biphenyl]-4-carboxamide;
2′-amino-5-chloro-2,4′-difluoro-N-(4-(methylsulfonyl)-3-(trifluoromethyl)phenyl)-[1,1′-biphenyl]-4-carboxamide;
2′-amino-5-chloro-2,4′-difluoro-N-(6-((tetrahydrofuran-3-yl)oxy)-5-(trifluoromethyl)pyridin-3-yl)-[1,1′-biphenyl]-4-carboxamide;
2′-amino-5-chloro-2,4′-difluoro-N-(6-(2-hydroxypropan-2-yl)-5-(trifluoromethyl)pyridin-3-yl)-[1,1′-biphenyl]-4-carboxamide;
5-(2′-amino-5-chloro-2,4′-difluoro-[1,1′-biphenyl]-4-carboxamido)-3-(trifluoromethyl)picolinamide;
2′-amino-5-chloro-N-(6-ethynyl-5-(trifluoromethyl)pyridin-3-yl)-2,4′-difluoro-[1,1′-biphenyl]-4-carboxamide;
2′-amino-5-chloro-2,4′-difluoro-N-(6-((tetrahydrofuran-3-yl)amino)-5-(trifluoromethyl)pyridin-3-yl)-[1,1′-biphenyl]-4-carboxamide;
2′-amino-5-chloro-N-(4-(cyclopropylsulfonyl)-3-(trifluoromethyl)phenyl)-2,4′-difluoro-[1,1′-biphenyl]-4-carboxamide;
2′-amino-5-chloro-2,4′-difluoro-N-(6-(pyrrolidine-1-carbonyl)-5-(trifluoromethyl)pyridin-3-yl)-[1,1′-biphenyl]-4-carboxamide;
2′-amino-5-chloro-N-(4-(N,N-dimethylsulfamoyl)-3-(trifluoromethyl)phenyl)-2,4′-difluoro-[1,1′-biphenyl]-4-carboxamide;
N-(4-(2-azaspiro[3.3]heptane-2-carbonyl)-3-(trifluoromethyl)phenyl)-2′-amino-5-chloro-2,4′-difluoro-[1,1′-biphenyl]-4-carboxamide;
2-chloro-N-(5-chloro-6-(1-oxo-2-oxa-7-azaspiro[4.4]nonan-7-yl)pyridin-3-yl)-4-(3-ethynylpyridin-4-yl)-5-fluorobenzamide;
2-chloro-N-(5-chloro-6-(2-oxa-7-azaspiro[4.4]nonan-7-yl)pyridin-3-yl)-4-(3-ethynylpyridin-4-yl)-5-fluorobenzamide;
2-chloro-N-(5-chloro-6-((1R,2S,4S)-2-(3-methylisoxazol-5-yl)-7-azabicyclo[2.2.1]heptan-7-yl)pyridin-3-yl)-4-(3-ethynylpyridin-4-yl)-5-fluorobenzamide;
2-chloro-N-(5-chloro-6-(1-cyano-7-azabicyclo[2.2.1]heptan-7-yl)pyridin-3-yl)-4-(3-ethynylpyridin-4-yl)-5-fluorobenzamide;
N-(6-(7-azabicyclo[2.2.1]heptan-7-yl)-5-chloropyridin-3-yl)-2-chloro-4-(3-ethynylpyridin-4-yl)-5-fluorobenzamide;
2-chloro-N-(5-chloro-6-(4-oxohexahydrocyclopenta[c]pyrrol-2(1H)-yl)pyridin-3-yl)-4-(3-ethynylpyridin-4-yl)-5-fluorobenzamide;
2-chloro-N-(5-chloro-6-((3aR,6aS)-5-oxohexahydrocyclopenta[c]pyrrol-2(1H)-yl)pyridin-3-yl)-4-(3-ethynylpyridin-4-yl)-5-fluorobenzamide;
2-chloro-N-(5-chloro-6-((3aR,6aS)-5,5-difluorohexahydrocyclopenta[c]pyrrol-2(1H)-yl)pyridin-3-yl)-4-(3-ethynylpyridin-4-yl)-5-fluorobenzamide;
2-chloro-N-(5-chloro-6-((2-oxopyrrolidin-1-yl)amino)pyridin-3-yl)-4-(3-ethynylpyridin-4-yl)-5-fluorobenzamide;
2-chloro-N-(5-chloro-6-(3-oxopyrazolidin-1-yl)pyridin-3-yl)-4-(3-ethynylpyridin-4-yl)-5-fluorobenzamide;
N-(6-(1H-benzo[d]imidazol-2-yl)-5-chloropyridin-3-yl)-2-chloro-4-(3-ethynylpyridin-4-yl)-5-fluorobenzamide;
2-chloro-N-(5-chloro-6-(4,5,6,7-tetrahydro-2H-[1,2,3]triazolo[4,5-c]pyridin-2-yl)pyridin-3-yl)-4-(3-ethynylpyridin-4-yl)-5-fluorobenzamide;
2-chloro-N-(5-chloro-6-(4,5-dicyano-2H-1,2,3-triazol-2-yl)pyridin-3-yl)-4-(3-ethynylpyridin-4-yl)-5-fluorobenzamide;
2-chloro-N-(5-chloro-6-(4,5,6,7-tetrahydro-2H-indazol-2-yl)pyridin-3-yl)-4-(3-ethynylpyridin-4-yl)-5-fluorobenzamide;
2-chloro-N-(5-chloro-6-(5,6-dihydrocyclopenta[c]pyrazol-2(4H)-yl)pyridin-3-yl)-4-(3-ethynylpyridin-4-yl)-5-fluorobenzamide;
2-chloro-N-(5-chloro-6-(3-(trifluoromethyl)-5,6-dihydrocyclopenta[c]pyrazol-2(4H)-yl)pyridin-3-yl)-4-(3-ethynylpyridin-4-yl)-5-fluorobenzamide;
ethyl 2-((3-chloro-5-(2-chloro-4-(3-ethynylpyridin-4-yl)-5-fluorobenzamido)pyridin-2-yl)amino)-2,4,5,6-tetrahydrocyclopenta[d][1,2,3]triazole-4-carboxylate;
N-(6-((2H-benzo[d][1,2,3]triazol-2-yl)amino)-5-chloropyridin-3-yl)-2-chloro-4-(3-ethynylpyridin-4-yl)-5-fluorobenzamide;
2-chloro-N-(5-chloro-6-(2-(trifluoromethyl)-5,6-dihydro-[1,2,4]triazolo[1,5-a]pyrazin-7(8H)-yl)pyridin-3-yl)-4-(3-ethynylpyridin-4-yl)-5-fluorobenzamide;
2-chloro-N-(5-chloro-6-(3-(trifluoromethyl)-5,6-dihydro-[1,2,4]triazolo[4,3-a]pyrazin-7(8H)-yl)pyridin-3-yl)-4-(3-ethynylpyridin-4-yl)-5-fluorobenzamide;
1-(3-chloro-5-(2-chloro-4-(3-ethynylpyridin-4-yl)-5-fluorobenzamido)pyridin-2-yl)-4,5,6,7-tetrahydro-1H-benzo[d]imidazole-5-carboxylic acid;
2-chloro-N-(2-(3,4-dichlorophenyl)benzo[d]oxazol-5-yl)-4-(3-ethynylpyridin-4-yl)-5-fluorobenzamide;
(S)-2-chloro-N-(5-chloro-6-(2-cyano-4,4-difluoropyrrolidine-1-carbonyl)pyridin-3-yl)-4-(3-ethynylpyridin-4-yl)-5-fluorobenzamide;
2-chloro-N-(5-chloro-6-((3aR,6aS)-hexahydro-1H-furo[3,4-c]pyrrole-5-carbonyl)pyridin-3-yl)-4-(3-ethynylpyridin-4-yl)-5-fluorobenzamide;
2-chloro-N-(5-chloro-6-(pyrrolidine-1-carbonyl)pyridin-3-yl)-4-(3-ethynylpyridin-4-yl)-5-fluorobenzamide;
2-chloro-N-(5-chloro-6-(4,4-difluoropiperidine-1-carbonyl)pyridin-3-yl)-4-(3-ethynylpyridin-4-yl)-5-fluorobenzamide;
2-chloro-N-(5-chloro-6-(6,6-difluoro-3-azabicyclo[3.1.0]hexane-3-carbonyl)pyridin-3-yl)-4-(3-ethynylpyridin-4-yl)-5-fluorobenzamide;
2-chloro-N-(5-chloro-6-(6,6-difluoro-3-azabicyclo[3.1.0]hexan-3-yl)pyridin-3-yl)-4-(3-ethynylpyridin-4-yl)-5-fluorobenzamide;
2-chloro-N-(5-chloro-6-(3-(4,4-difluoropiperidin-1-yl)azetidin-1-yl)pyridin-3-yl)-4-(3-ethynylpyridin-4-yl)-5-fluorobenzamide;
2-chloro-N-(5-chloro-6-(3-(4,4-difluoropiperidin-1-yl)azetidine-1-carbonyl)pyridin-3-yl)-4-(3-ethynylpyridin-4-yl)-5-fluorobenzamide;
4,4′-dichloro-N-(5-chloro-6-(2H-1,2,3-triazol-2-yl)pyridin-3-yl)-6-(3-ethynylpyridin-4-yl)-[1,1′-biphenyl]-3-carboxamide;
2-amino-4″,5′-dichloro-N-(5-chloro-6-(2H-1,2,3-triazol-2-yl)pyridin-3-yl)-4-fluoro-[1,1′:2′,1″-terphenyl]-4′-carboxamide;
N-(6-(2H-1,2,3-triazol-2-yl)-5-(trifluoromethyl)pyridin-3-yl)-5′-methyl-[1,1′:2′,1″-terphenyl]-4′-carboxamide;
2-chloro-N-(5-chloro-6-(2H-1,2,3-triazol-2-yl)pyridin-3-yl)-5-ethynyl-4-(3-ethynylpyridin-4-yl)benzamide;
N-(5-chloro-6-(2H-1,2,3-triazol-2-yl)pyridin-3-yl)-5-ethynyl-4-(3-ethynylpyridin-4-yl)-2-methylbenzamide;
N-(5-chloro-6-(2H-1,2,3-triazol-2-yl)pyridin-3-yl)-5-cyclopropyl-4-(3-ethynylpyridin-4-yl)-2-methylbenzamide;
4-(3-amino-5-fluoropyridin-2-yl)-N-(5-chloro-6-(2H-1,2,3-triazol-2-yl)pyridin-3-yl)-5-cyclopropyl-2-methylbenzamide;
2-chloro-N-(5-chloro-6-(2H-1,2,3-triazol-2-yl)pyridin-3-yl)-5-cyclopropyl-4-(3-ethynylpyridin-4-yl)benzamide;
4-(3-amino-5-fluoropyridin-2-yl)-2-chloro-N-(5-chloro-6-(2H-1,2,3-triazol-2-yl)pyridin-3-yl)-5-cyclopropylbenzamide;
2′-amino-N-(5-chloro-6-(2H-1,2,3-triazol-2-yl)pyridin-3-yl)-2-ethynyl-4′-fluoro-5-methyl-[1,1′-biphenyl]-4-carboxamide;
N-(6-(2H-1,2,3-triazol-2-yl)-5-(trifluoromethyl)pyridin-3-yl)-4-(3-amino-5-ethynylpyridin-4-yl)-2-chloro-5-fluorobenzamide;
4-(3-amino-5-ethynylpyridin-4-yl)-2-chloro-N-(5-chloro-6-(2H-1,2,3-triazol-2-yl)pyridin-3-yl)-5-fluorobenzamide;
2′-amino-5-chloro-N-(5-chloro-6-(2H-1,2,3-triazol-2-yl)pyridin-3-yl)-6′-ethynyl-2,4′-difluoro-[1,1′-biphenyl]-4-carboxamide;
N-(6-(2H-1,2,3-triazol-2-yl)-5-(trifluoromethyl)pyridin-3-yl)-2′,5-dichloro-6′-ethynyl-2-fluoro-[1,1′-biphenyl]-4-carboxamide;
2′-chloro-N-(5-chloro-6-(2H-1,2,3-triazol-2-yl)pyridin-3-yl)-2-ethynyl-4′-fluoro-5-methyl-[1,1′-biphenyl]-4-carboxamide;
N-(5-chloro-6-(2H-1,2,3-triazol-2-yl)pyridin-3-yl)-2,2′-diethynyl-4′-fluoro-5-methyl-[1,1′-biphenyl]-4-carboxamide;
4-(2-amino-6-fluoropyridin-3-yl)-N-(5-chloro-6-(2H-1,2,3-triazol-2-yl)pyridin-3-yl)-5-ethynyl-2-methylbenzamide;
N-(5-chloro-6-(2H-1,2,3-triazol-2-yl)pyridin-3-yl)-5-ethynyl-4-(3-methoxypyridin-4-yl)-2-methylbenzamide;
2-amino-5′-chloro-N-(5-chloro-6-(2H-1,2,3-triazol-2-yl)pyridin-3-yl)-4-fluoro-2″,3″,4″,5″-tetrahydro-[1,1′:2′,1″-terphenyl]-4′-carboxamide;
2-chloro-N-(5-chloro-6-(2H-1,2,3-triazol-2-yl)pyridin-3-yl)-4-fluoro-5′-methyl-[1,1′:2′,1″-terphenyl]-4′-carboxamide;
2′-amino-N-(5-chloro-6-(2H-1,2,3-triazol-2-yl)pyridin-3-yl)-2-(cyclopent-1-en-1-yl)-4′-fluoro-5-methyl-[1,1′-biphenyl]-4-carboxamide;
2-amino-N-(5-chloro-6-(2H-1,2,3-triazol-2-yl)pyridin-3-yl)-4-fluoro-5′-methyl-[1,1′:2′,1″-terphenyl]-4′-carboxamide;
N-(5-chloro-6-(2H-1,2,3-triazol-2-yl)pyridin-3-yl)-2-ethynyl-4-fluoro-5′-methyl-[1,1′:2′,1″-terphenyl]-4′-carboxamide;
2′-amino-N-(5-chloro-6-(2H-1,2,3-triazol-2-yl)pyridin-3-yl)-4′-fluoro-5-methyl-2-(prop-1-yn-1-yl)-[1,1′-biphenyl]-4-carboxamide;
2′-amino-5-chloro-N-(5-chloro-6-(2H-1,2,3-triazol-2-yl)pyridin-3-yl)-4′-fluoro-2-vinyl-[1,1′-biphenyl]-4-carboxamide;
2′-amino-N-(5-chloro-6-(2H-1,2,3-triazol-2-yl)pyridin-3-yl)-4′-fluoro-5-methyl-2-vinyl-[1,1′-biphenyl]-4-carboxamide;
3-chloro-5-(2-chloro-4-(3-ethynylpyridin-4-yl)-5-fluorobenzamido)-N-methylpicolinamide;
3-chloro-5-(2-chloro-4-(3-ethynylpyridin-4-yl)-5-fluorobenzamido)-N-(cyanomethyl)picolinamide;
3-chloro-5-(2-chloro-4-(3-ethynylpyridin-4-yl)-5-fluorobenzamido)-N-ethylpicolinamide;
3-chloro-5-(2-chloro-4-(3-ethynylpyridin-4-yl)-5-fluorobenzamido)-N-(2,2,2-trifluoroethyl)picolinamide;
3-chloro-5-(2-chloro-4-(3-ethynylpyridin-4-yl)-5-fluorobenzamido)-N-propylpicolinamide;
3-chloro-5-(2-chloro-4-(3-ethynylpyridin-4-yl)-5-fluorobenzamido)-N-(2-(trifluoromethoxy)ethyl)picolinamide;
3-chloro-5-(2-chloro-4-(3-ethynylpyridin-4-yl)-5-fluorobenzamido)-N-phenethylpicolinamide;
3-chloro-5-(2-chloro-4-(3-ethynylpyridin-4-yl)-5-fluorobenzamido)-N-(furan-3-ylmethyl)picolinamide;
3-chloro-5-(2-chloro-4-(3-ethynylpyridin-4-yl)-5-fluorobenzamido)-N-neopentylpicolinamide;
3-chloro-5-(2-chloro-4-(3-ethynylpyridin-4-yl)-5-fluorobenzamido)-N-(2-(diethylamino)-2-oxoethyl)picolinamide;
5-(2′-amino-5-chloro-2,4′-difluoro-[1,1′-biphenyl]-4-carboxamido)-3-chloro-N-((1-methylcyclopropyl)methyl)picolinamide;
5-(2′-amino-5-chloro-2,4′-difluoro-[1,1′-biphenyl]-4-carboxamido)-3-chloro-N-((1,1-dioxidotetrahydro-2H-thiopyran-4-yl)methyl)picolinamide;
5-(2′-amino-5-chloro-2,4′-difluoro-[1,1′-biphenyl]-4-carboxamido)-N-butyl-3-chloropicolinamide;
5-(2′-amino-5-chloro-2,4′-difluoro-[1,1′-biphenyl]-4-carboxamido)-3-chloro-N-(prop-2-yn-1-yl)picolinamide;
N-(6-(2H-1,2,3-triazol-2-yl)-5-(trifluoromethyl)pyridin-3-yl)-4-(3-bromo-5-nitropyridin-4-yl)-2-chloro-5-fluorobenzamide;
5-(2′-amino-5-chloro-2,4′-difluoro-[1,1′-biphenyl]-4-carboxamido)-3-chloro-N-(cyanomethyl)picolinamide;
5-(2′-amino-5-chloro-2,4′-difluoro-6′-methyl-[1,1′-biphenyl]-4-carboxamido)-3-chloro-N-(cyanomethyl)picolinamide;
5-(2′-amino-5-chloro-2,4′-difluoro-[1,1′-biphenyl]-4-carboxamido)-3-chloro-N-(2,2,2-trifluoroethyl)picolinamide;
5-(2′-amino-5-chloro-2,4′-difluoro-[1,1′-biphenyl]-4-carboxamido)-3-chloro-N-ethylpicolinamide;
2-chloro-N-(5-chloro-6-(2H-1,2,3-triazol-2-yl)pyridin-3-yl)-4-(3-cyclopropylpyridin-4-yl)-5-fluorobenzamide;
5-(2′-amino-5-chloro-2,4′-difluoro-[1,1′-biphenyl]-4-carboxamido)-3-chloro-N-neopentylpicolinamide;
5-(2′-amino-5-chloro-2,4′-difluoro-[1,1′-biphenyl]-4-carboxamido)-3-chloro-N-(furan-3-ylmethyl)picolinamide;
5-(2′-amino-5-chloro-2,4′-difluoro-[1,1′-biphenyl]-4-carboxamido)-3-chloro-N-propylpicolinamide;
5-(2′-amino-5-chloro-2,4′-difluoro-[1,1′-biphenyl]-4-carboxamido)-3-chloro-N-(2-methoxyethyl)picolinamide;
5-(2′-amino-5-chloro-2,4′-difluoro-[1,1′-biphenyl]-4-carboxamido)-3-chloro-N-(1-cyanoethyl)picolinamide;
5-(2′-amino-5-chloro-2,4′-difluoro-[1,1′-biphenyl]-4-carboxamido)-3-chloro-N-((3,3-difluorocyclobutyl)methyl)picolinamide;
N-((1,3,4-oxadiazol-2-yl)methyl)-5-(2′-amino-5-chloro-2,4′-difluoro-[1,1′-biphenyl]-4-carboxamido)-3-chloropicolinamide;
5-(2′-amino-5-chloro-2,4′-difluoro-[1,1′-biphenyl]-4-carboxamido)-3-chloro-N-(2-(dimethylamino)ethyl)picolinamide;
5-(2′-amino-5-chloro-2,4′-difluoro-[1,1′-biphenyl]-4-carboxamido)-3-chloro-N-(cyclobutylmethyl)picolinamide;
5-(2′-amino-5-chloro-2,4′-difluoro-[1,1′-biphenyl]-4-carboxamido)-3-chloro-N-((1-methyl-1H-pyrazol-3-yl)methyl)picolinamide;
5-(4-(2-amino-6-fluoropyridin-3-yl)-2-chloro-5-fluorobenzamido)-3-chloro-N-(cyanomethyl)picolinamide;
5-(2′-amino-5-chloro-2,4′-difluoro-[1,1′-biphenyl]-4-carboxamido)-3-chloro-N-(2,2-difluoro-2λ3-ethyl)picolinamide;
5-(2′-amino-5-chloro-2,4′-difluoro-[1,1′-biphenyl]-4-carboxamido)-3-chloro-N-(2,2,3,3,4,4,4-heptafluorobutyl)picolinamide;
5-(2′-amino-5-chloro-2,4′-difluoro-[1,1′-biphenyl]-4-carboxamido)-3-chloro-N-(2-(trifluoromethoxy)ethyl)picolinamide;
2,2′-diamino-5-chloro-N-(5-chloro-6-(2H-1,2,3-triazol-2-yl)pyridin-3-yl)-4′-fluoro-[1,1′-biphenyl]-4-carboxamide;
5-(2′-amino-5-chloro-2,4′-difluoro-[1,1′-biphenyl]-4-carboxamido)-3-chloro-N-(2-fluoroethyl)picolinamide;
5-(2′-amino-5-chloro-2,4′-difluoro-[1,1′-biphenyl]-4-carboxamido)-3-chloro-N-((5-methyl-1,3,4-oxadiazol-2-yl)methyl)picolinamide;
N-((1H-1,2,4-triazol-3-yl)methyl)-5-(2′-amino-5-chloro-2,4′-difluoro-[1,1′-biphenyl]-4-carboxamido)-3-chloropicolinamide;
5-(2′-amino-5-chloro-2,4′-difluoro-[1,1′-biphenyl]-4-carboxamido)-3-chloro-N-(2-(ethylthio)ethyl)picolinamide;
N-(2-amino-2-oxoethyl)-5-(2′-amino-5-chloro-2,4′-difluoro-[1,1′-biphenyl]-4-carboxamido)-3-chloropicolinamide;
5-(2′-amino-5-chloro-2,4′-difluoro-[1,1′-biphenyl]-4-carboxamido)-3-chloro-N-((1-cyanocyclopropyl)methyl)picolinamide;
5-(2′-amino-5-chloro-2,4′-difluoro-[1,1′-biphenyl]-4-carboxamido)-3-chloro-N-(2,2,3,3,3-pentafluoropropyl)picolinamide;
5-(2′-amino-5-chloro-2,4′-difluoro-[1,1′-biphenyl]-4-carboxamido)-3-chloro-N-((1-(trifluoromethyl)cyclopropyl)methyl)picolinamide;
5-(2′-amino-5-chloro-2,4′-difluoro-[1,1′-biphenyl]-4-carboxamido)-3-chloro-N-(2-(methylsulfonyl)ethyl)picolinamide;
5-(4-(3-amino-5-ethynylpyridin-4-yl)-2-chloro-5-fluorobenzamido)-3-chloro-N-(2,2,2-trifluoroethyl)picolinamide;
5-(4-(3-amino-5-ethynylpyridin-4-yl)-2-chloro-5-fluorobenzamido)-3-chloro-N-(2,2-difluoroethyl)picolinamide;
5-(4-(3-amino-5-ethynylpyridin-4-yl)-2-chloro-5-fluorobenzamido)-3-chloro-N-(2-fluoroethyl)picolinamide;
5-(4-(3-amino-5-ethynylpyridin-4-yl)-2-chloro-5-fluorobenzamido)-3-chloro-N-((1,1-dioxidotetrahydro-2H-thiopyran-4-yl)methyl)picolinamide;
5-(4-(3-amino-5-ethynylpyridin-4-yl)-2-chloro-5-fluorobenzamido)-3-chloro-N-(furan-3-ylmethyl)picolinamide;
N-(6-(2H-1,2,3-triazol-2-yl)-5-(trifluoromethyl)pyridin-3-yl)-4-(3-amino-5-cyclopropylpyridin-4-yl)-2-chloro-5-fluorobenzamide;
5-(4-(3-amino-5-ethynylpyridin-4-yl)-2-chloro-5-fluorobenzamido)-3-chloro-N-(1-cyanoethyl)picolinamide;
5-(4-(3-amino-5-ethynylpyridin-4-yl)-2-chloro-5-fluorobenzamido)-3-chloro-N-(cyclopropylmethyl)picolinamide;
5-(4-(3-amino-5-ethynylpyridin-4-yl)-2-chloro-5-fluorobenzamido)-3-chloro-N-neopentylpicolinamide;
5-(4-(3-amino-5-ethynylpyridin-4-yl)-2-chloro-5-fluorobenzamido)-3-chloro-N-ethylpicolinamide;
5-(4-(3-amino-5-ethynylpyridin-4-yl)-2-chloro-5-fluorobenzamido)-3-chloro-N-(1-cyanocyclopropyl)picolinamide;
5-(4-(3-amino-5-ethynylpyridin-4-yl)-2-chloro-5-fluorobenzamido)-3-chloro-N-((1-cyanocyclopropyl)methyl)picolinamide;
5-(4-(3-amino-5-ethynylpyridin-4-yl)-2-chloro-5-fluorobenzamido)-3-chloro-N-(2-(methylsulfonyl)ethyl)picolinamide;
5-(4-(3-amino-5-ethynylpyridin-4-yl)-2-chloro-5-fluorobenzamido)-3-chloro-N-(2-(ethylthio)ethyl)picolinamide;
5-(4-(3-amino-5-ethynylpyridin-4-yl)-2-chloro-5-fluorobenzamido)-3-chloro-N-((5-methyl-1,3,4-oxadiazol-2-yl)methyl)picolinamide;
4-(3-amino-5-ethynyl-2-fluoropyridin-4-yl)-2-chloro-N-(5-chloro-6-(2H-1,2,3-triazol-2-yl)pyridin-3-yl)-5-fluorobenzamide;
4-(5-amino-3-ethynyl-2-fluoropyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-fluoropyridin-2-yl)-2-chloro-5-ethynylbenzamide;
N-(6-(2H-1,2,3-triazol-2-yl)-5-(trifluoromethyl)pyridin-3-yl)-4-(3-amino-5-(2-methylprop-1-en-1-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(1-ethoxyvinyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(1-ethoxyethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(1-hydroxyethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(2-cyanoethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(2-ethoxyethyl)pyridin-4-yl)-2-chloro-5-fluorobenzamide;
N-(6-(2H-1,2,3-triazol-2-yl)-5-(trifluoromethyl)pyridin-3-yl)-4-(3-acetyl-5-aminopyridin-4-yl)-2-chloro-5-fluorobenzamide;
N-(6-(2H-1,2,3-triazol-2-yl)-5-(trifluoromethyl)pyridin-3-yl)-4-(3-amino-5-(3,3,3-trifluoroprop-1-en-2-yl)pyridin-4-yl)-2-chloro-5-fluorobenzamide;
(E)-N-(6-(2H-1,2,3-triazol-2-yl)-5-(trifluoromethyl)pyridin-3-yl)-4-(3-amino-5-(3-hydroxy-3-methylbut-1-en-1-yl)pyridin-4-yl)-2-chloro-5-fluorobenzamide;
(E)-N-(6-(2H-1,2,3-triazol-2-yl)-5-(trifluoromethyl)pyridin-3-yl)-4-(3-amino-5-(2-cyclopropylvinyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(2-cyclopropylethyl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(3-hydroxy-3-methylbutyl)pyridin-4-yl)-2-chloro-5-fluorobenzamide;
(E)-N-(6-(2H-1,2,3-triazol-2-yl)-5-(trifluoromethyl)pyridin-3-yl)-4-(3-amino-5-(3-methoxyprop-1-en-1-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(3-methoxypropyl)pyridin-4-yl)-2-chloro-5-fluorobenzamide;
5-(4-(3-amino-5-ethynylpyridin-4-yl)-2-chloro-5-fluorobenzamido)-3-chloro-N-(2,2,2-trifluoroethyl)picolinamide;
4-(3-amino-5-(1-(difluoromethyl)-1H-pyrazol-4-yl)pyridin-4-yl)-2-chloro-N-(5-chloro-6-(2H-1,2,3-triazol-2-yl)pyridin-3-yl)-5-fluorobenzamide;
4-(3-amino-5-(prop-1-en-2-yl)pyridin-4-yl)-2-chloro-N-(5-chloro-6-(2H-1,2,3-triazol-2-yl)pyridin-3-yl)-5-fluorobenzamide;
4-(3-amino-5-ethynylpyridin-4-yl)-2-chloro-N-(5-ethynyl-6-(2H-1,2,3-triazol-2-yl)pyridin-3-yl)-5-fluorobenzamide;
4-(3-amino-5-cyclopropylpyridin-4-yl)-2-chloro-N-(5-chloro-6-(2H-1,2,3-triazol-2-yl)pyridin-3-yl)-5-fluorobenzamide;
4-(3-amino-5-vinylpyridin-4-yl)-2-chloro-N-(5-chloro-6-(2H-1,2,3-triazol-2-yl)pyridin-3-yl)-5-fluorobenzamide;
4-(3-amino-5-(prop-1-yn-1-yl)pyridin-4-yl)-2-chloro-N-(5-chloro-6-(2H-1,2,3-triazol-2-yl)pyridin-3-yl)-5-fluorobenzamide formate;
4-(3-amino-5-(cyclohex-1-en-1-yl)pyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-bromo-2-ethynylpyridin-4-yl)-2-chloro-5-fluorobenzamide;
N-(6-(2H-1,2,3-triazol-2-yl)-5-(trifluoromethyl)pyridin-3-yl)-4-(3-amino-2,5-diethynylpyridin-4-yl)-2-chloro-5-fluorobenzamide;
4-(3-amino-5-(cyclopent-1-en-1-yl)pyridin-4-yl)-2-chloro-N-(5-chloro-6-(2H-1,2,3-triazol-2-yl)pyridin-3-yl)-5-fluorobenzamide;
4-(3-amino-5-ethynylpyridin-4-yl)-2-chloro-5-fluoro-N-(6-(trifluoromethyl)pyridin-3-yl)benzamide;
4-(3-amino-5-(cyclopropylethynyl)pyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-ethynylpyridin-4-yl)-3-fluorobenzamide;
5-(4-(3-amino-5-cyclopropylpyridin-4-yl)-2-chloro-5-fluorobenzamido)-3-chloro-N-(2,2,2-trifluoroethyl)picolinamide;
N-(6-(2H-1,2,3-triazol-2-yl)-5-(trifluoromethyl)pyridin-3-yl)-4-(3-amino-5-bromopyridin-4-yl)-2-chloro-5-fluorobenzamide;
4-(3-amino-5-ethynylpyridin-4-yl)-2-chloro-5-fluoro-N-(2-(trifluoromethyl)pyridin-4-yl)benzamide;
5-(4-(3-amino-5-cyclopropylpyridin-4-yl)-2-chloro-5-fluorobenzamido)-3-cyclopropyl-N-(2,2,2-trifluoroethyl)picolinamide;
N-(6-(2H-1,2,3-triazol-2-yl)-5-(trifluoromethyl)pyridin-3-yl)-4-(5-amino-3-ethynyl-2-fluoropyridin-4-yl)-2-chloro-5-fluorobenzamide;
N-(6-(2H-1,2,3-triazol-2-yl)-5-(trifluoromethyl)pyridin-3-yl)-4-(5-amino-3-bromo-2-fluoropyridin-4-yl)-2-chloro-5-fluorobenzamide;
N-(6-(2H-1,2,3-triazol-2-yl)-5-(trifluoromethyl)pyridin-3-yl)-4-(3-amino-5-(3,6-dihydro-2H-pyran-4-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-(3,6-dihydro-2H-pyran-4-yl)pyridin-4-yl)-3-fluorobenzamide;
4-(3-amino-5-cyclopropylpyridin-4-yl)-2-chloro-5-fluoro-N-(2-(trifluoromethyl)pyridin-4-yl)benzamide;
2′-amino-5-chloro-N-(5-cyclopropyl-6-(2H-1,2,3-triazol-2-yl)pyridin-3-yl)-2,4′-difluoro-[1,1′-biphenyl]-4-carboxamide;
2-chloro-4-(3-cyclopropyl-5-(methylamino)pyridin-4-yl)-5-fluoro-N-(2-(trifluoromethyl)pyridin-4-yl)benzamide;
N-(6-(2H-1,2,3-triazol-2-yl)-5-(trifluoromethyl)pyridin-3-yl)-2′-amino-5-chloro-6′-cyclopropyl-2,4′-difluoro-[1,1′-biphenyl]-4-carboxamide;
N-(6-(2H-1,2,3-triazol-2-yl)-5-(trifluoromethyl)pyridin-3-yl)-4-(3-amino-5-(2,5-dihydrofuran-3-yl)pyridin-4-yl)-2-chloro-5-fluorobenzamide;
4-(3-acetyl-5-aminopyridin-4-yl)-2-chloro-N-(5-chloro-6-(2H-1,2,3-triazol-2-yl)pyridin-3-yl)-5-fluorobenzamide;
N-(6-(2H-1,2,3-triazol-2-yl)-5-(trifluoromethyl)pyridin-3-yl)-4-(3-amino-5-(4-hydroxycyclohex-1-en-1-yl)pyridin-4-yl)-2-chloro-5-fluorobenzamide;
N-(6-(2H-1,2,3-triazol-2-yl)-5-(trifluoromethyl)pyridin-3-yl)-4-(3-amino-5-phenylpyridin-4-yl)-2-chloro-5-fluorobenzamide;
2′-amino-5-chloro-6′-(2-ethoxyethyl)-2,4′-difluoro-N-(2-(trifluoromethyl)pyridin-4-yl)-[1,1′-biphenyl]-4-carboxamide;
2′-amino-5-chloro-2,4′-difluoro-6′-(tetrahydrofuran-2-yl)-N-(2-(trifluoromethyl)pyridin-4-yl)-[1,1′-biphenyl]-4-carboxamide;
2′-amino-5-chloro-2,4′-difluoro-6′-(4-morpholinocyclohexyl)-N-(2-(trifluoromethyl)pyridin-4-yl)-[1,1′-biphenyl]-4-carboxamide;
2′-amino-5-chloro-2,4′-difluoro-6′-(4-hydroxycyclohexyl)-N-(2-(trifluoromethyl)pyridin-4-yl)-[1,1′-biphenyl]-4-carboxamide;
2′-amino-5-chloro-2,4′-difluoro-6′-(2-oxo-1,2-dihydropyridin-4-yl)-N-(2-(trifluoromethyl)pyridin-4-yl)-[1,1′-biphenyl]-4-carboxamide;
2′-amino-5-chloro-2,4′-difluoro-6′-(6-oxo-1,6-dihydropyridin-3-yl)-N-(2-(trifluoromethyl)pyridin-4-yl)-[1,1′-biphenyl]-4-carboxamide;
2′-amino-5-chloro-2,4′-difluoro-6′-(1-methylpiperidin-3-yl)-N-(2-(trifluoromethyl)pyridin-4-yl)-[1,1′-biphenyl]-4-carboxamide;
2′-(1-acetylpiperidin-4-yl)-6′-amino-5-chloro-2,4′-difluoro-N-(2-(trifluoromethyl)pyridin-4-yl)-[1,1′-biphenyl]-4-carboxamide;
2′-amino-5-chloro-2,4′-difluoro-6′-(spiro[2.5]octan-6-yl)-N-(2-(trifluoromethyl)pyridin-4-yl)-[1,1′-biphenyl]-4-carboxamide;
2′-amino-5-chloro-2,4′-difluoro-6′-(2-oxo-1,2-dihydropyrimidin-5-yl)-N-(2-(trifluoromethyl)pyridin-4-yl)-[1,1′-biphenyl]-4-carboxamide;
2′-amino-5-chloro-2,4′-difluoro-6′-(1-(2,2,2-trifluoroacetyl)piperidin-4-yl)-N-(2-(trifluoromethyl)pyridin-4-yl)-[1,1′-biphenyl]-4-carboxamide;
2′-amino-5-chloro-2,4′-difluoro-6′-(4-(trifluoromethyl)cyclohexyl)-N-(2-(trifluoromethyl)pyridin-4-yl)-[1,1′-biphenyl]-4-carboxamide;
2′-amino-5-chloro-2,4′-difluoro-6′-(1-hydroxyethyl)-N-(2-(trifluoromethyl)pyridin-4-yl)-[1,1′-biphenyl]-4-carboxamide;
4-(3-amino-5-bromo-2-chloropyridin-4-yl)-2-chloro-5-fluoro-N-(2-(trifluoromethyl)pyridin-4-yl)benzamide;
4-(3-amino-2,5-dimethylpyridin-4-yl)-2-chloro-5-fluoro-N-(2-(trifluoromethyl)pyridin-4-yl)benzamide;
4-(3-amino-5-bromo-2-methoxypyridin-4-yl)-2-chloro-5-fluoro-N-(2-(trifluoromethyl)pyridin-4-yl)benzamide;
2-chloro-4-(2,3-diamino-5-ethynylpyridin-4-yl)-5-fluoro-N-(2-(trifluoromethyl)pyridin-4-yl)benzamide;
4-(2-amino-5-ethynyl-3-iodopyridin-4-yl)-2-chloro-5-fluoro-N-(2-(trifluoromethyl)pyridin-4-yl)benzamide;
4-(3-amino-5-ethynyl-2-methylpyridin-4-yl)-2-chloro-5-fluoro-N-(2-(trifluoromethyl)pyridin-4-yl)benzamide;
N-(6-(2H-1,2,3-triazol-2-yl)-5-(trifluoromethyl)pyridin-3-yl)-4-(3-amino-5-ethynyl-2-methylpyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2-fluoropyridin-4-yl)-2-chloro-5-fluorobenzamide;
N-(6-(2H-1,2,3-triazol-2-yl)-5-(trifluoromethyl)pyridin-3-yl)-4-(3-amino-2-chloro-5-ethynylpyridin-4-yl)-2-chloro-5-fluorobenzamide;
N-(6-(2H-1,2,3-triazol-2-yl)-5-(trifluoromethyl)pyridin-3-yl)-4-(3-amino-2-cyano-5-ethynylpyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2-methoxypyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-6-fluoro-2-methylpyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2,6-difluoropyridin-4-yl)-2-chloro-5-fluorobenzamide;
N-(6-(2H-1,2,3-triazol-2-yl)-5-(trifluoromethyl)pyridin-3-yl)-4-(3-amino-5-bromo-2-chloropyridin-4-yl)-2-chloro-5-fluorobenzamide;
N-(6-(2H-1,2,3-triazol-2-yl)-5-(trifluoromethyl)pyridin-3-yl)-4-(3-amino-5-ethynyl-2-hydroxypyridin-4-yl)-2-chloro-5-hydroxybenzamide;
N-(6-(2H-1,2,3-triazol-2-yl)-5-(trifluoromethyl)pyridin-3-yl)-4-(3-amino-5-bromo-2-hydroxypyridin-4-yl)-2-chloro-5-hydroxybenzamide;
4-(4-((6-(2H-1,2,3-triazol-2-yl)-5-(trifluoromethyl)pyridin-3-yl)carbamoyl)-5-chloro-2-fluorophenyl)-3-amino-5-ethynylpicolinamide;
N-(6-(2H-1,2,3-triazol-2-yl)-5-(trifluoromethyl)pyridin-3-yl)-4-(3-amino-5-ethynylpyridazin-4-yl)-2-chloro-5-fluorobenzamide;
N-(6-(2H-1,2,3-triazol-2-yl)-5-(trifluoromethyl)pyridin-3-yl)-4-(2-amino-4-ethynyl-6-fluoropyridin-3-yl)-2-chloro-5-fluorobenzamide;
N-(6-(2H-1,2,3-triazol-2-yl)-5-(trifluoromethyl)pyridin-3-yl)-4-(2-amino-4-ethynylpyridin-3-yl)-2-chloro-5-fluorobenzamide;
N-((1S)-1-(4-(4-(3-amino-5-ethynylpyridin-4-yl)-2-chloro-5-fluorobenzamide)phenyl)-2,2,2-trifluoroethyl)-N-methyltetrahydro2H-thiopyran-4-carboxamide1,1-dioxide;
4-(3-amino-2-chloro-5-ethynylpyridin-4-yl)-2-chloro-N-(5-chloro-6-(2H-1,2,3-triazol-2-yl)pyridin-3-yl)-5-fluorobenzamide;
4-(3-amino-2-chloro-5-ethynylpyridin-4-yl)-2-chloro-5-fluoro-N-(2-(trifluoromethyl)pyridin-4-yl)benzamido;
4-(3-amino-5-ethynylpyridin-4-yl)-2-chloro-5-fluoro-N-(5-hydroxy-6-(2H-1,2,3-triazol-2-yl)pyridin-3-yl)benzamido;
4-(3-amino-5-ethynylpyridin-4-yl)-2-chloro-5-fluoro-N-(5-methoxy-6-(2H-1,2,3-triazol-2-yl)pyridin-3-yl)benzamido;
4-(3-amino-5-ethynylpyridin-4-yl)-2-chloro-N-(5-(difluoromethoxy)-6-(2H-1,2,3-triazol-2-yl)pyridin-3-yl)-5-fluorobenzamide; or
N-(6-(2H-1,2,3-triazol-2-yl)-5-(trifluoromethyl)pyridin-3-yl)-4-(5-amino-3-ethynyl-2-fluoropyridin-4-yl)-2-chloro-5-fluorobenzamide.

18. A pharmaceutical composition comprising a therapeutically effective amount of at least a compound of claim 1, or a stereoisomer, tautomer, deuterated compound, pharmaceutically acceptable salt, prodrug, chelate, non-covalent complex or solvate thereof as an active ingredient, and at least one pharmaceutically acceptable ingredient.

19.-21. (canceled)

22. A method of treating diseases, syndromes, disorders or obstacles, wherein the diseases, syndromes, disorders and obstacles are affected by MALT1 inhibition, comprising administering to a patient in need the compound of claim 1, or a stereoisomer, tautomer, deuterated compound, pharmaceutically acceptable salt, prodrug, chelate, non-covalent complex or solvate thereof.

23. The method of claim 22, wherein the diseases, syndromes, disorders and obstacles are selected from diffuse large B-cell lymphoma (DLBCL), mantle cell lymphoma (MCL), follicular lymphoma (FL), mucosa-associated lymphoid tissue (MALT) lymphoma, rheumatoid arthritis (RA), psoriatic arthritis (PsA), psoriasis (Pso), ulcerative colitis (UC), Crohn's disease, systemic lupus erythematosus (SLE), asthma or chronic obstructive pulmonary disease (COPD).
